(19) United States
(12) Patent Application Publication
(10) Pub. No.: US 2018/0194762 A1
(43) Pub. Date: Jul. 12, 2018

(54) PYRAZOLO[3,4-b]PYRIDINE AND PYRROLO[2,3-b]PYRIDINE INHIBITORS OF BRUTON'S TYROSINE KINASE

(71) Applicant: Pharmacyclics LLC, Sunnyvale, CA (US)

(72) Inventors: Gordana B. Atallah, Fremont, CA (US); Wei Chen, Fremont, CA (US); Dimitry Khrakovsky, Fremont, CA (US); Longcheng Wang, Palo Alto, CA (US); Zhaozhong Jon Jia, San Mateo, CA (US); Felix DeAnda, JR., Sunnyvale, CA (US)

(21) Appl. No.: 15/864,884

(22) Filed: Jan. 8, 2018

Related U.S. Application Data
(60) Provisional application No. 62/443,498, filed on Jan. 6, 2017.

(51) Int. Cl. C07D 471/04; C07D 519/00; A61P 35/00; A61P 29/00

(52) U.S. Cl. CPC C07D 471/04; A61P 29/00; A61P 35/00; C07D 519/00

(57) ABSTRACT

Disclosed are pyrazolo[3,4-b]pyridine and pyrrolo[2,3-b]pyridine inhibitors of Bruton's tyrosine kinase (Btk). Also disclosed are pharmaceutical compositions that include the compounds. Methods of using the Btk inhibitors are described, alone or in combination with other therapeutic agents, for the treatment of autoimmune diseases or conditions, heteroimmune diseases or conditions, cancer, including lymphoma, and inflammatory diseases or conditions.

### REFERENCE TO RELATED APPLICATIONS

This application claims the benefit of U.S. Provisional Patent Application No. 62/443,498, filed Jan. 6, 2017, the contents of which are hereby incorporated by reference herein in their entirety.

### FIELD OF THE INVENTION

Described herein are compounds, methods of making such compounds, pharmaceutical compositions and medicaments containing such compounds, and methods of using such compounds and compositions to inhibit the activity of tyrosine kinases.

### BACKGROUND OF THE INVENTION

Bruton's tyrosine kinase (Btk), a member of the Tec family of non-receptor tyrosine kinases, is a key signaling enzyme expressed in all hematopoietic cells types except T lymphocytes and natural killer cells. Btk plays an essential role in the B-cell signaling pathway linking cell surface B-cell receptor (BCR) stimulation to downstream intracellular responses. Btk is a key regulator of B-cell development, activation, signaling, and survival (Kurosaki, Curr Op Imm, 2000, 276-281; Schaeffer and Schwartzberg, Curr Op Imm 2000, 282-288). In addition, Btk plays a role in a number of other hematopoetic cell signaling pathways, e.g., Toll like receptor (TLR) and cytokine receptor-mediated TNF-α production in macrophages, IgE receptor (FcepsilonRI) signaling in Mast cells, inhibition of Fas/APO-1 apoptotic signaling in B-lineage lymphoid cells, and collagen-stimulated platelet aggregation. See, e.g., C. A. Jeffries, et al., (2003), Journal of Biological Chemistry 278:26258-26264; N. J. Horwood, et al., (2003), The Journal of Experimental Medicine 197:1603-1611; Iwaki et al. (2005), Journal of Biological Chemistry 280(48):40261-40270; Vassilev et al. (1999), Journal of Biological Chemistry 274(3): 1646-1656, and Quek et al. (1998), Current Biology 8(20): 1137-1140.

### SUMMARY OF THE INVENTION

Described herein are inhibitors of Bruton's tyrosine kinase (Btk). Also described herein are irreversible inhibitors of Btk. Also described herein are reversible inhibitors of Btk. Further described are irreversible inhibitors of Btk that form a covalent bond with a cysteine residue on Btk. Further described herein are irreversible inhibitors of other tyrosine kinases, wherein the other tyrosine kinases share homology with Btk by having a cysteine residue (including a Cys 481 residue) that can form a covalent bond with the irreversible inhibitor (such tyrosine kinases, are referred herein as “Btk tyrosine kinase cysteine homologs”).

Also described herein are methods for synthesizing such reversible or irreversible inhibitors, methods for using such reversible or irreversible inhibitors in the treatment of diseases (including diseases wherein irreversible inhibition of Btk provides therapeutic benefit to a patient having the disease). Further described are pharmaceutical formulations that include a reversible or irreversible inhibitor of Btk.

In one aspect, provided herein is a compound of Formula (I) having the structure:

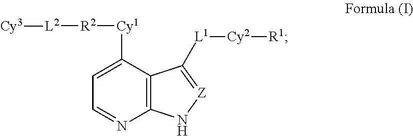

or a pharmaceutically acceptable salt thereof; or a stereoisomer or an isotopic variant thereof;
wherein:
Z is C(R⁹) or N;
R⁹ is H, halo, substituted or unsubstituted C₁-C₆alkyl, OR^{13a}, —NR^{13a}R^{13b}, —SR^{13a}, C₁-C₄alkoxyC₁-C₄alkyl, hydroxyC₁-C₄alkyl, haloC₁-C₄alkyl, haloC₁-C₄alkoxy, cyano, substituted or unsubstituted C₃-C₈cycloalkyl, substituted or unsubstituted C₂-C₇heterocycloalkyl, substituted or unsubstituted aryl, or substituted or unsubstituted heteroaryl;
each R^{13a} and R^{13b} is independently H, substituted or unsubstituted C₁-C₆alkyl, or substituted or unsubstituted C₃-C₈cycloalkyl;
Cy¹ is substituted or unsubstituted aryl or substituted or unsubstituted heteroaryl;
Cy² is substituted or unsubstituted C₃-C₈cycloalkyl, substituted or unsubstituted C₂-C₇heterocycloalkyl, substituted or unsubstituted C₂-C₇heterocycloalkenyl, or substituted or unsubstituted C₃-C₈cycloalkenyl;
Cy³ is substituted or unsubstituted C₃-C₈cycloalkyl, substituted or unsubstituted C₂-C₇heterocycloalkyl, substituted or unsubstituted C₂-C₇heterocycloalkenyl, or substituted or unsubstituted C₃-C₈cycloalkenyl, substituted or unsubstituted aryl, or substituted or unsubstituted heteroaryl;
L¹ is a single bond, substituted or unsubstituted C₁-C₄alkylene, —N(R⁵)—, —O—, or —S—;
R⁵ is H, substituted or unsubstituted C₁-C₆alkyl, substituted or unsubstituted C₃-C₈cycloalkyl, or —C(O)—R^{5a};
R^{5a} is substituted or unsubstituted C₁-C₄alkyl, substituted or unsubstituted C₃-C₈cycloalkyl, substituted or unsubstituted C₂-C₇heterocycloalkyl, substituted or unsubstituted aryl, or substituted or unsubstituted heteroaryl;
L² is —N(R^{10a})C(O)—, —C(O)N(R^{10a})—, —N(R^{10a})C(O)N(R^{10b}), —O—, —S—, —S(O)—, —S(O)ₚ—, —N(R^{10a})S(O)ₚ—, or —S(O)ₚN(R^{10a})—; or L² and Cy³, taken together with the atoms to which they are attached, form a 9-14 membered bicyclic or tricyclic heterocyclyl which is unsubstituted or substituted with one or more substituents selected from C₁-C₆ alkyl, C₃-C₆ cycloalkyl, hydroxy, and carbonyl;
each R^{10a} and R^{10b} is independently H, substituted or unsubstituted C₁-C₆alkyl, or substituted or unsubstituted C₃-C₈cycloalkyl;
p is 1 or 2;
R¹ is H, halo, substituted or unsubstituted C₁-C₆alkyl, —OR^{12a}, —NR^{12a}R^{12b}, —SR^{12a}, —C(O)—O—R^{12a}, —C(O)—C(O)N(R^{12a})R^{12b}, —C(O)—N(R^{12a})R^{12b}, —S(O)ₚ—N(R^{12a})R^{12b}, C₁-C₄alkoxyC₁-C₄alkyl, hydroxyC₁-C₄alkyl, haloC₁-C₄alkyl, haloC₁-C₄alkoxy, cyano, substituted or unsubstituted C₃-C₈cycloalkyl, substituted or unsubstituted C₂-C₇heterocycloalkyl, substituted or unsubstituted aryl, or substituted or unsubstituted heteroaryl;
each R^{12a} and R^{12b} is independently H, substituted or unsubstituted C₁-C₆alkyl, or substituted or unsubstituted C₃-C₈cycloalkyl; or
R¹ is —C(O)—R^{1a}, —C(S)—R^{1a}, —S(O)_{q}—R^{1a}; —N(R^{12a})—C(O)R^{1a}, or —N(R^{12a})—S(o)_{q}R^{1a};
R^{1a} is H, substituted or unsubstituted C₁-C₄alkyl, substituted or unsubstituted C₂-C₄alkenyl, substituted or unsubstituted C₂-C₄alkynyl, substituted or unsubstituted C₃-C₈cycloalkyl, substituted or unsubstituted C₂-C₇heterocycloalkyl, substituted or unsubstituted aryl, or substituted or unsubstituted heteroaryl;
q is 1 or 2; and
R² is a single bond, substituted or unsubstituted C₁-C₄alkylene, or substituted or unsubstituted C₃-C₆cycloalkylene.

In some embodiments, when R⁹ is substituted or unsubstituted C₁-C₆alkyl, the substituents are selected from halo, hydroxyl, and alkoxy. In some embodiments, R⁹ is unsubstituted.

In some embodiments, when R⁹ is substituted or unsubstituted C₃-C₈cycloalkyl, substituted or unsubstituted C₂-C₇heterocycloalkyl, substituted or unsubstituted aryl, or substituted or unsubstituted heteroaryl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R⁹ is unsubstituted.

In some embodiments of Formula (I), when R^{13a} or R^{13b} is substituted or unsubstituted C₁-C₆alkyl, the substituents are selected from halo, hydroxyl, and alkoxy. In some embodiments, R^{13a} or R^{13b} is unsubstituted.

In some embodiments, when R^{13a} or R^{13b} is substituted or unsubstituted C₃-C₈cycloalkyl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R^{13a} or R^{13b} is unsubstituted.

In some embodiments, the substituents on Cy¹ are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents on Cy¹ are selected from Cl, F, CN, Me, Et, t-Bu, CHF₂, CF₃, cyclopropyl, hydroxyl, hydroxymethyl, or methoxy. In some embodiments, Cy¹ is unsubstituted.

In some embodiments, the substituents on Cy² are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents on Cy² are selected from Cl, F, CN, Me, Et, t-Bu, CHF₂, CF₃, cyclopropyl, hydroxyl, hydroxymethyl, or methoxy. In some embodiments, Cy² is unsubstituted.

In some embodiments, the substituents on Cy³ are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents on Cy³ are selected from Cl, F, CN, Me, Et, t-Bu, CHF₂, CF₃, cyclopropyl, hydroxyl, hydroxymethyl, or methoxy. In some embodiments, Cy³ is unsubstituted.

In some embodiments, when L¹ is substituted or unsubstituted C₁-C₄alkylene, the substituents are selected from halo, hydroxyl, and alkoxy. In some embodiments, L¹ is unsubstituted.

In some embodiments, when R⁵ is substituted or unsubstituted C₁-C₆alkyl, the substituents are selected from halo, hydroxyl, and alkoxy. In some embodiments, R⁵ is unsubstituted.

In some embodiments, when R⁵ is substituted or unsubstituted C₃-C₈cycloalkyl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R⁵ is unsubstituted.

In some embodiments, when R^{5a} is substituted or unsubstituted C₁-C₄alkyl, the substituents are selected from halo, hydroxyl, and alkoxy. In some embodiments, R^{5a} is unsubstituted.

In some embodiments, when R^{5a} is substituted or unsubstituted C₃-C₈cycloalkyl, substituted or unsubstituted C₂-C₇heterocycloalkyl, substituted or unsubstituted aryl, or substituted or unsubstituted heteroaryl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R^{5a} is unsubstituted.

In some embodiments, when R^{10a} or R^{10b} is substituted or unsubstituted C₁-C₆alkyl, the substituents are selected from halo, hydroxyl, and alkoxy. In some embodiments, R^{10a} or R^{10b} is unsubstituted.

In some embodiments, when R^{10a} or R^{10b} is substituted or unsubstituted C₃-C₈cycloalkyl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R^{10a} or R^{10b} is unsubstituted.

In some embodiments, when R¹ is substituted or unsubstituted C₁-C₄alkyl, the substituents are selected from halo, hydroxyl, and alkoxy. In some embodiments, R¹ is unsubstituted.

In some embodiments, when R¹ is substituted or unsubstituted C₃-C₈cycloalkyl, substituted or unsubstituted C₂-C₇heterocycloalkyl, substituted or unsubstituted aryl, or substituted or unsubstituted heteroaryl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R¹ is unsubstituted.

In some embodiments, when R^{12a} or R^{12b} is substituted or unsubstituted C₁-C₆alkyl, the substituents are selected from halo, hydroxyl, and alkoxy. In some embodiments, R^{12a} or R^{12b} is unsubstituted.

In some embodiments, when R^{12a} or R^{12b} is substituted or unsubstituted C₃-C₈cycloalkyl, the substituents are selected from halo, CN, C₁-C₄alkyl, haloC₁-C₄alkyl, hydroxyC₁-C₄alkyl, C₃-C₈cycloalkyl, hydroxyl, amino, alkylamino, and C₁-C₄alkoxy. In some embodiments, when R^{12a} or R^{12b} is substituted or unsubstituted C₃-C₈cycloalkyl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, NH₂, Me, Et, t-Bu, CHF₂, CF₃, cyclopropyl, hydroxyl, hydroxymethyl, or methoxy. In some embodiments, R^{12a} or R^{12b} is unsubstituted.

In some embodiments, when R^{12a} or R^{12b} is substituted or unsubstituted C₁-C₆alkyl, the substituents are selected from halo, CN, C₁-C₄alkyl, haloC₁-C₄alkyl, hydroxyC₁-C₄alkyl, C₃-C₈cycloalkyl, hydroxyl, amino, alkylamino, and C₁-C₄alkoxy. In some embodiments, when R^{12a} or R^{12b} is substituted or unsubstituted C₃-C₈cycloalkyl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, NH₂, Me, Et, t-Bu, CHF₂, CF₃, cyclopropyl, hydroxyl, hydroxymethyl, or methoxy. In some embodiments, R^{12a} or R^{12b} is unsubstituted.

In some embodiments, when R^{1a} is substituted or unsubstituted C₁-C₄alkyl, substituted or unsubstituted C₂-C₄alkenyl, or substituted or unsubstituted C₂-C₄alkynyl, the substituents are selected from halo, hydroxyl, and alkoxy. In some embodiments, R^{1a} is unsubstituted.

In some embodiments, when R^{1a} is substituted or unsubstituted C₃-C₈cycloalkyl, substituted or unsubstituted C₂-C₇heterocycloalkyl, substituted or unsubstituted aryl, or substituted or unsubstituted heteroaryl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R^{1a} is unsubstituted.

In some embodiments, when R² is substituted or unsubstituted C₁-C₄alkylene, the substituents are selected from halo, hydroxyl, and alkoxy. In some embodiments, R² is unsubstituted.

In some embodiments, when R² is substituted or unsubstituted C₃-C₆cycloalkylene, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R² is unsubstituted.

In some embodiments, L² and Cy³, taken together with the atoms to which they are attached, form a 9-14 membered bicyclic or tricyclic heterocyclyl which is unsubstituted or substituted with one or more substituents selected from C₁-C₆ alkyl, C₃-C₆ cycloalkyl, hydroxy, and carbonyl. In some embodiments, the L²-Cy³ moiety is selected from those shown in the following structural formulas:

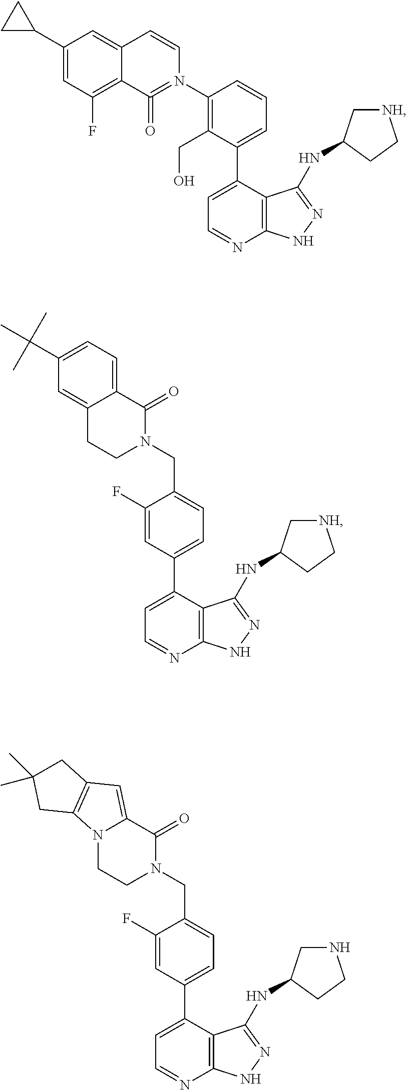

In some embodiments, L²-Cy³ is:

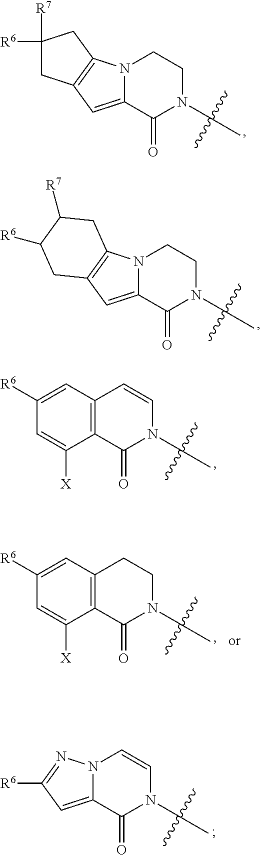

wherein:
each instance of R¹⁴ is independently selected from the group consisting of H, C₁₋₆ alkyl, and C₃₋₆ cycloalkyl; and
X is halo.

In some embodiments, L²-Cy³ is:

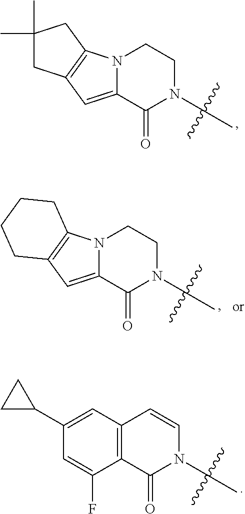 [SMILES: *N1CCn2c(cc3c2CC(C)(C)C3)C1=O ; *N1CCn2c(cc3c2CCCC3)C1=O ; *n1ccc2cc(C3CC3)cc(F)c2c1=O]

In one embodiment, —R²-L²-Cy³ is —R²—N(R^{10a})C(O)-Cy³, —R²—C(O)N(R^{10a})-Cy³, or —R²—O-Cy³. In another embodiment, —R²-L²-Cy³ is —R²—N(H)C(O)-Cy³.

In one aspect, provided herein is a compound of Formula (II) having the structure:

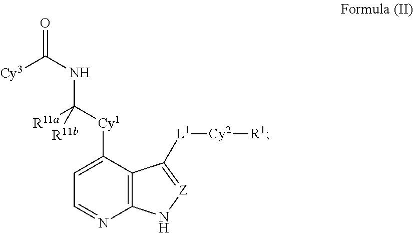

or a pharmaceutically acceptable salt thereof; or a stereoisomer or an isotopic variant thereof;
wherein
Cy¹, Cy², Cy³, L¹, R¹ and Z are as described for Formula (I);
each R^{11a} and R^{11b} is independently H or substituted or unsubstituted C₁-C₄alkyl; or
R^{11a} and R^{11b} may join together with the carbon atom to which they are attached to form a substituted or unsubstituted C₃-C₆cycloalkylene; and wherein the substitutions on R^{11a} and R^{11b}, if present, are independently selected from —OH, halo, and C₁-C₄alkyl.

In some embodiments, when R^{11a} or R^{11b} is substituted or unsubstituted C₁-C₄alkyl, the substituents are selected from halo, hydroxyl, and alkoxy. In some embodiments, R^{11a} or R^{11b} is unsubstituted.

In one aspect, provided herein is a compound of Formula (III) having the structure:

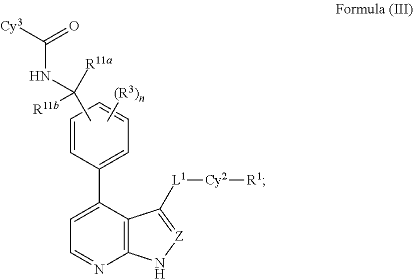

or a pharmaceutically acceptable salt thereof; or a stereoisomer or an isotopic variant thereof;
wherein
Cy², Cy³, L¹, R¹ and Z are as described for Formula (I);
each R^{11a} and R^{11b} is independently H or substituted or unsubstituted C₁-C₄alkyl; or
R^{11a} and R^{11b} may join together with the carbon atom to which they are attached to form a substituted or unsubstituted C₃-C₆cycloalkylene; and wherein the substitutions on R^{11a} and R^{11b}, if present, are independently selected from —OH, halo, or C₁-C₄alkyl;
each R³ is each independently halo, substituted or unsubstituted C₁-C₆alkyl, —OR^{21a}, —NR^{21a}R^{21b}, —SR^{21a}, —C(O)—O—R^{21a}, —C(O)—C(O)—N(R^{21a})R^{21b}, —C(O)—N(R^{21a})R^{21b}, —N(R^{21a})C(O)—R^{21b}, —S(O)ₜ—N(R^{21a})R^{21b}, C₁-C₄alkoxyC₁-C₄alkyl, hydroxyC₁-C₄alkyl, haloC₁-C₄alkyl, haloC₁-C₄alkoxy, cyano, substituted or unsubstituted C₃-C₈cycloalkyl, substituted or unsubstituted C₂-C₇heterocycloalkyl, substituted or unsubstituted aryl, or substituted or unsubstituted heteroaryl;
each R^{21a} and R^{21b} is independently H, substituted or unsubstituted C₁-C₆alkyl, or substituted or unsubstituted C₃-C₈cycloalkyl; and
n is 0-4.

In some embodiments, the compound of Formula (III) has the structure:

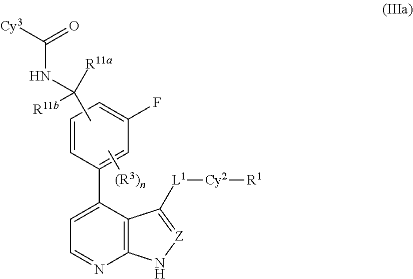

In some embodiments, when R^{11a} or R^{11b} is substituted or unsubstituted C₁-C₄alkyl, the substituents are selected from halo, hydroxyl, and alkoxy. In some embodiments, R^{11a} or R^{11b} is unsubstituted.

In some embodiments, when R³ is substituted or unsubstituted C₁-C₆alkyl, the substituents are selected from halo, hydroxyl, and alkoxy. In some embodiments, R³ is unsubstituted.

In some embodiments, when R³ is substituted or unsubstituted C₃-C₈cycloalkyl, substituted or unsubstituted C₂-C₇heterocycloalkyl, substituted or unsubstituted aryl, or substituted or unsubstituted heteroaryl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R³ is unsubstituted.

In some embodiments, when R^{21a} or R^{21b} is substituted or unsubstituted C₁-C₆alkyl, the substituents are selected from halo, hydroxyl, and alkoxy. In some embodiments, R^{21a} or R^{21b} is unsubstituted.

In some embodiments, when R^{21a} or R^{21b} is substituted or unsubstituted C₃-C₈cycloalkyl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R^{21a} or R^{21b} is unsubstituted.

In one aspect, provided herein is a compound of Formula (IVa) or (IVb) having the structure:

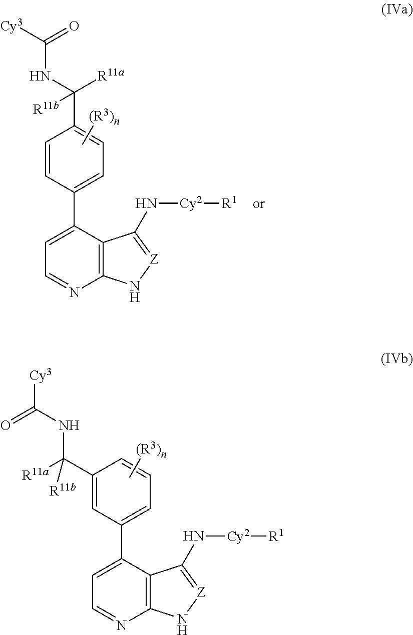

or a pharmaceutically acceptable salt thereof; or a stereoisomer or an isotopic variant thereof;
wherein
Cy², Cy³, R¹ and Z are as described for Formula (I);
each R^{11a} and R^{11b} is independently H or substituted or unsubstituted C₁-C₄alkyl; or
R^{11a} and R^{11b} may join together with the carbon atom to which they are attached to form a substituted or unsubstituted C₃-C₆cycloalkylene; and wherein the substitutions on R^{11a} and R^{11b}, if present, are independently selected from —OH, halo, or C₁-C₄alkyl;
each R³ is each independently halo, substituted or unsubstituted C₁-C₆alkyl, —OR^{21a}, —NR^{21a}R^{21b}, —SR^{21a}, —C(O)—O—R^{21a}, —C(O)—C(O)—N(R^{21a})R^{21b}, —C(O)—N(R^{21a})R^{21b}, —N(R^{21a})C(O)—R^{21b}, —S(O)ₜ—N(R^{21a})R^{21b}, C₁-C₄alkoxyC₁-C₄alkyl, hydroxyC₁-C₄alkyl, haloC₁-C₄alkyl, haloC₁-C₄alkoxy, cyano, substituted or unsubstituted C₃-C₈cycloalkyl, substituted or unsubstituted C₂-C₇heterocycloalkyl, substituted or unsubstituted aryl, or substituted or unsubstituted heteroaryl;
each R^{21a} and R^{21b} is independently H, substituted or unsubstituted C₁-C₆alkyl, or substituted or unsubstituted C₃-C₈cycloalkyl; and
n is 0-4.

In some embodiments, when R^{11a} or R^{11b} is substituted or unsubstituted C₁-C₄alkyl, the substituents are selected from halo, hydroxyl, and alkoxy. In some embodiments, R^{11a} or R^{11b} is unsubstituted.

In some embodiments, when R³ is substituted or unsubstituted C₁-C₆alkyl, the substituents are selected from halo, hydroxyl, and alkoxy. In some embodiments, R³ is unsubstituted.

In some embodiments, when R³ is substituted or unsubstituted C₃-C₈cycloalkyl, substituted or unsubstituted C₂-C₇heterocycloalkyl, substituted or unsubstituted aryl, or substituted or unsubstituted heteroaryl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R³ is unsubstituted.

In some embodiments, when R^{21a} or R^{21b} is substituted or unsubstituted C₁-C₆alkyl, the substituents are selected from halo, hydroxyl, and alkoxy. In some embodiments, R^{21a} or R^{21b} is unsubstituted.

In some embodiments, when R^{21a} or R^{21b} is substituted or unsubstituted C₃-C₈cycloalkyl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R^{21a} or R^{21b} is unsubstituted.

In one aspect, provided herein is a compound of Formula (Va), (Vb), (Vc), or (Vd), having the structure:

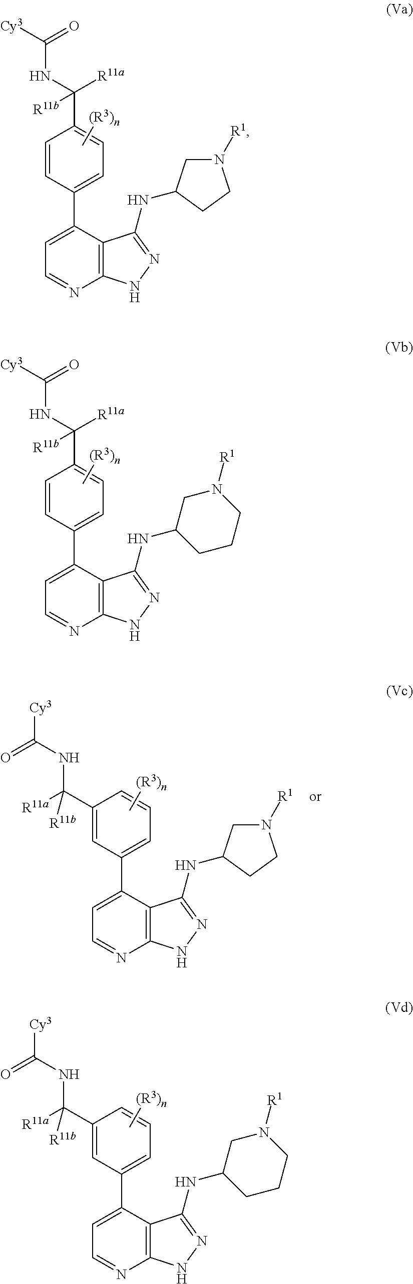

or a pharmaceutically acceptable salt thereof; or a stereoisomer or an isotopic variant thereof;
wherein
Cy³, R¹ and Z are as described for Formula (I);
each R^{11a} and R^{11b} is independently H or substituted or unsubstituted C₁-C₄alkyl; or
R^{11a} and R^{11b} may join together with the carbon atom to which they are attached to form a substituted or unsubstituted C₃-C₆cycloalkylene; and wherein the substitutions on R^{11a} and Rub, if present, are independently selected from —OH, halo, or C₁-C₄alkyl;
each R³ is each independently halo, substituted or unsubstituted C₁-C₆alkyl, —OR^{21a}, —NR^{21a}R^{21b}, —SR^{21a}, —C(O)—O—R^{21a}, —C(O)—C(O)—N(R^{21a})R^{21b}, —C(O)—N(R^{21a})R^{21b}, —N(R^{21a})C(O)—R^{21b}, —S(O)ₜ—N(R^{21a})R^{21b}, C₁-C₄alkoxyC₁-C₄alkyl, hydroxyC₁-C₄alkyl, haloC₁-C₄alkyl, haloC₁-C₄alkoxy, cyano, substituted or unsubstituted C₃-C₈cycloalkyl, substituted or unsubstituted C₂-C₇heterocycloalkyl, substituted or unsubstituted aryl, or substituted or unsubstituted heteroaryl;
each R^{21a} and R^{21b} is independently H, substituted or unsubstituted C₁-C₆alkyl, or substituted or unsubstituted C₃-C₈cycloalkyl; and
n is 0-4.

In some embodiments, when R^{11a} or R^{11b} is substituted or unsubstituted C₁-C₄alkyl, the substituents are selected from halo, hydroxyl, and alkoxy. In some embodiments, R^{11a} or R^{11b} is unsubstituted.

In some embodiments, when R³ is substituted or unsubstituted C₁-C₆alkyl, the substituents are selected from halo, hydroxyl, and alkoxy. In some embodiments, R³ is unsubstituted.

In some embodiments, when R³ is substituted or unsubstituted C₃-C₈cycloalkyl, substituted or unsubstituted C₂-C₇heterocycloalkyl, substituted or unsubstituted aryl, or substituted or unsubstituted heteroaryl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R³ is unsubstituted.

In some embodiments, when R^{21a} or R^{21b} is substituted or unsubstituted C₁-C₆alkyl, the substituents are selected from halo, hydroxyl, and alkoxy. In some embodiments, R^{21a} or R^{21b} is unsubstituted.

In some embodiments, when R^{21a} or R^{21b} is substituted or unsubstituted C₃-C₈cycloalkyl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R^{21a} or R^{21b} is unsubstituted.

In one aspect, provided herein is a compound of Formula (VIa), (VIb), (VIc), or (VId), having the structure:

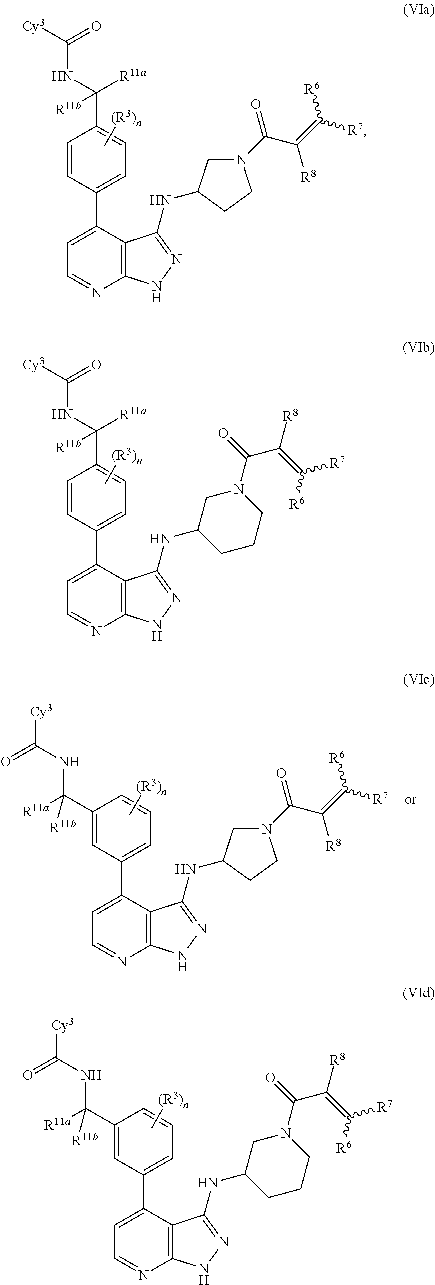

or a pharmaceutically acceptable salt thereof; or a stereoisomer or an isotopic variant thereof;
wherein
Cy³, and Z are as described for Formula (I);
each R^{11a} and R^{11b} is independently H or substituted or unsubstituted C₁-C₄alkyl; or
R^{11a} and R^{11b} may join together with the carbon atom to which they are attached to form a substituted or unsubstituted C₃-C₆cycloalkylene; and wherein the substitutions on R^{11a} and R^{11b}, if present, are independently selected from —OH, halo, or C₁-C₄alkyl;
each R³ is each independently halo, substituted or unsubstituted C₁-C₆alkyl, —OR^{21a}, —NR^{21a}R^{21b}, —SR^{21a}, —C(O)—O—R^{21a}, —C(O)—C(O)—N(R^{21a})R^{2b}, —C(O)—N(R^{21a})R^{21b}, —N(R^{21a})C(O)—R^{21b}, —S(O)ₜ—N(R^{21a})R^{21b}, C₁-C₄alkoxyC₁-C₄alkyl, hydroxyC₁-C₄alkyl, haloC₁-C₄alkyl, haloC₁-C₄alkoxy, cyano, substituted or unsubstituted C₃-C₈cycloalkyl, substituted or unsubstituted C₂-C₇heterocycloalkyl, substituted or unsubstituted aryl, or substituted or unsubstituted heteroaryl;
each R^{21a} and R^{21b} is independently H, substituted or unsubstituted C₁-C₆alkyl, or substituted or unsubstituted C₃-C₈cycloalkyl;
n is 0-4; and
R⁶, R⁷ and R⁸ are each independently H, CN, halo, substituted or unsubstituted C₁-C₄alkyl, substituted or unsubstituted C₃-C₅cycloalkyl, substituted or unsubstituted C₂-C₇heterocycloalkyl, substituted or unsubstituted C₆-C₁₂aryl, or substituted or unsubstituted 5- to 8-membered heteroaryl; or
R⁷ and R⁸ together form a bond.

In some embodiments, when R^{11a} or R^{11b} is substituted or unsubstituted C₁-C₄alkyl, the substituents are selected from halo, hydroxyl, and alkoxy. In some embodiments, R^{11a} or R^{11b} is unsubstituted.

In some embodiments, when R³ is substituted or unsubstituted C₁-C₆alkyl, the substituents are selected from halo, hydroxyl, and alkoxy. In some embodiments, R³ is unsubstituted.

In some embodiments, when R³ is substituted or unsubstituted C₃-C₈cycloalkyl, substituted or unsubstituted C₂-C₇heterocycloalkyl, substituted or unsubstituted aryl, or substituted or unsubstituted heteroaryl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R³ is unsubstituted.

In some embodiments, when R^{21a} or R^{21b} is substituted or unsubstituted C₁-C₆alkyl, the substituents are selected from halo, hydroxyl, and alkoxy. In some embodiments, R^{21a} or R^{21b} is unsubstituted.

In some embodiments, when R^{21a} or R^{21b} is substituted or unsubstituted C₃-C₈cycloalkyl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R^{21a} or R^{21b} is unsubstituted.

In some embodiments, when R⁶, R⁷ or R⁸ is substituted or unsubstituted C₁-C₄alkyl, the substituents are selected from halo, hydroxyl, and alkoxy. In some embodiments, R⁶, R⁷ or R⁸ is unsubstituted.

In some embodiments, when R⁶, R⁷ or R⁸ is substituted or unsubstituted C₃-C₈cycloalkyl, substituted or unsubstituted C₂-C₇heterocycloalkyl, substituted or unsubstituted C₆-C₁₂aryl, or substituted or unsubstituted 5- to 8-membered heteroaryl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R⁶, R⁷ or R⁸ is unsubstituted.

In one aspect, provided herein is a compound of Formula (VIIa), (VIIb), (VIIc), or (VIId), having the structure:

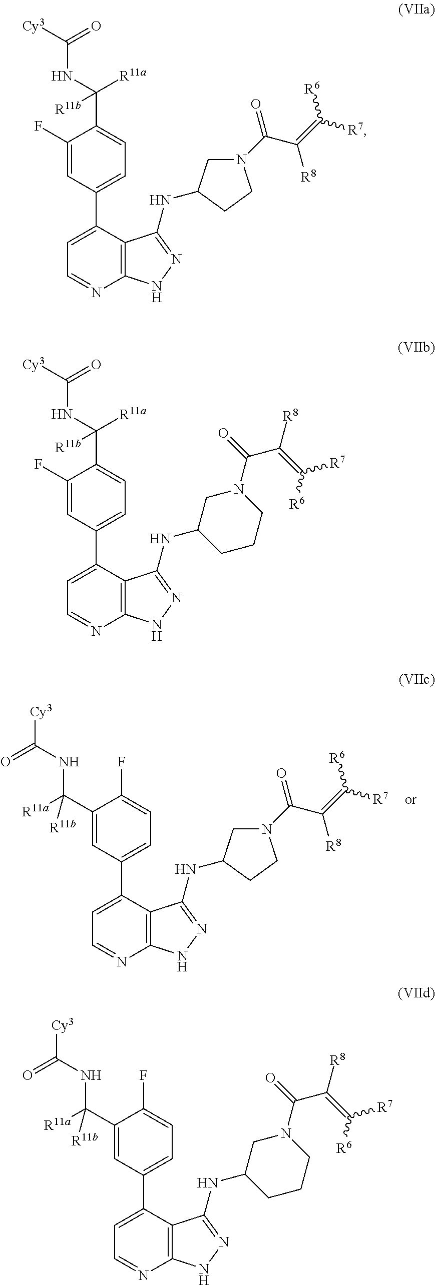

or a pharmaceutically acceptable salt thereof, or a stereoisomer or an isotopic variant thereof;
wherein
Cy³ is as described for Formula (I);
each R^{11a} and R^{11b} is independently H or substituted or unsubstituted C₁-C₄alkyl; or
R^{11a} and R^{11b} may join together with the carbon atom to which they are attached to form a substituted or unsubstituted C₃-C₆cycloalkylene; and wherein the substitutions on R^{11a} and R^{11b}, if present, are independently selected from —OH, halo, or C₁-C₄alkyl; and
R⁶, R⁷ and R⁸ are each independently H, CN, halo, substituted or unsubstituted C₁-C₄alkyl, substituted or unsubstituted C₃-C₅cycloalkyl, substituted or unsubstituted C₂-C₇heterocycloalkyl, substituted or unsubstituted C₆-C₁₂aryl, or substituted or unsubstituted 5- to 8-membered heteroaryl; or
R⁷ and R⁸ together form a bond.

In some embodiments, when R^{11a} or R^{11b} is substituted or unsubstituted C₁-C₄alkyl, the substituents are selected from halo, hydroxyl, and alkoxy. In some embodiments, R^{11a} or R^{11b} is unsubstituted.

In some embodiments, when R³ is substituted or unsubstituted C₁-C₆alkyl, the substituents are selected from halo, hydroxyl, and alkoxy. In some embodiments, R³ is unsubstituted.

In some embodiments, when R³ is substituted or unsubstituted C₃-C₈cycloalkyl, substituted or unsubstituted C₂-C₇heterocycloalkyl, substituted or unsubstituted aryl, or substituted or unsubstituted heteroaryl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R³ is unsubstituted.

In some embodiments, when R^{21a} or R^{21b} is substituted or unsubstituted C₁-C₆alkyl, the substituents are selected from halo, hydroxyl, and alkoxy. In some embodiments, R^{21a} or R^{21b} is unsubstituted.

In some embodiments, when R^{21a} or R^{21b} is substituted or unsubstituted C₃-C₈cycloalkyl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R^{21a} or R^{21b} is unsubstituted.

In some embodiments, when R⁶, R⁷ or R⁸ is substituted or unsubstituted C₁-C₄alkyl, the substituents are selected from halo, hydroxyl, and alkoxy. In some embodiments, R⁶, R⁷ or R⁸ is unsubstituted.

In some embodiments, when R⁶, R⁷ or R⁸ is substituted or unsubstituted C₃-C₈cycloalkyl, substituted or unsubstituted C₂-C₇heterocycloalkyl, substituted or unsubstituted C₆-C₁₂aryl, or substituted or unsubstituted 5- to 8-membered heteroaryl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R⁶, R⁷ or R⁸ is unsubstituted.

Another aspect of the invention is a pharmaceutical composition comprising a therapeutically effective amount of a compound of Formula (I), (II), (III), (IIIa), (IVa)-(IVd), (Va)-(Vd), (VIa)-(VId) or (VIIa)-(VIId) or a pharmaceutically acceptable salt thereof; or a stereoisomer or an isotopic variant thereof, and one or more pharmaceutically acceptable excipients. In one embodiment, the pharmaceutical composition comprising the compound of Formula (I), (II), (III), (IIIa), (IVa)-(IVd), (Va)-(Vd), (VIa)-(VId) or (VIIa)-(VIId) or a pharmaceutically acceptable salt thereof; or a stereoisomer or an isotopic variant thereof, is formulated for a route of administration selected from oral administration, parenteral administration, buccal administration, nasal administration, topical administration, or rectal administration.

In another aspect, the present invention is a method for treating an autoimmune disease or condition comprising administering to a patient in need a therapeutically effective amount of a compound of Formula (I), (II), (III), (IIIa), (IVa)-(IVd), (Va)-(Vd), (VIa)-(VId) or (VIIa)-(VIId) or a pharmaceutically acceptable salt thereof; or a stereoisomer or an isotopic variant thereof. In some embodiments, the autoimmune disease is selected from rheumatoid arthritis or lupus.

In a further aspect, the present invention is a method for treating a heteroimmune disease or condition comprising administering to a patient in need a therapeutically effective amount of a compound of Formula (I), (II), (III), (IIIa), (IV) or (V) or a pharmaceutically acceptable salt thereof; or a stereoisomer or an isotopic variant thereof.

In some embodiments, the present invention is a method for treating a cancer comprising administering to a patient in need a therapeutically effective amount of a compound of Formula (I), (II), (III), (IIIa), (IVa)-(IVd), (Va)-(Vd), (VIa)-(VId) or (VIIa)-(VIId) or a pharmaceutically acceptable salt thereof; or a stereoisomer or an isotopic variant thereof. In some embodiments, the cancer is a B-cell proliferative disorder. In some embodiments, the B-cell proliferative disorder is diffuse large B cell lymphoma, follicular lymphoma, mantle cell lymphoma, or chronic lymphocytic leukemia.

In yet a further aspect the present invention is a method for treating mastocytosis comprising administering to a patient in need a therapeutically effective amount of a compound of Formula (I), (II), (III), (IIIa), (IVa)-(IVd), (Va)-(Vd), (VIa)-(VId) or (VIIa)-(VIId) or a pharmaceutically acceptable salt thereof; or a stereoisomer or an isotopic variant thereof.

In another aspect the present invention is a method for treating osteoporosis or bone resorption disorders comprising administering to a patient in need a therapeutically effective amount of a compound of Formula (I), (II), (III), (IIIa), (IVa)-(IVd), (Va)-(Vd), (VIa)-(VId) or (VIIa)-(VIId) or a pharmaceutically acceptable salt thereof; or a stereoisomer or an isotopic variant thereof.

In another aspect the present invention is a method for treating an inflammatory disease or condition comprising administering to a patient in need a therapeutically effective amount of a compound of Formula (I), (II), (III), (IIIa), (IVa)-(IVd), (Va)-(Vd), (VIa)-(VId) or (VIIa)-(VIId) or a pharmaceutically acceptable salt thereof; or a stereoisomer or an isotopic variant thereof.

Each and every possible combination of the groups described above for the various variables is contemplated herein. It is understood that the compounds provided herein can be synthesized by techniques known in the art, as well as those set forth herein.

In a further aspect are provided pharmaceutical compositions, which include a therapeutically effective amount of at least one of any of the compounds herein, or a pharmaceutically acceptable salt or stereoisomer thereof. In certain embodiments, compositions provided herein further include one or more pharmaceutically acceptable diluents, excipients and/or binders.

Pharmaceutical compositions may be formulated for administration by an appropriate route and means containing effective concentrations of one or more of the compounds provided herein, or pharmaceutically effective derivatives thereof, that deliver amounts effective for the treatment or amelioration of one or more symptoms of diseases, disorders or conditions that are modulated or otherwise affected by tyrosine kinase activity, or in which tyrosine kinase activity is implicated. Suitable effective amounts and concentrations are those effective for ameliorating any of the symptoms of any of the diseases, disorders or conditions disclosed herein.

In certain embodiments, provided herein is a pharmaceutical composition containing: i) one or more physiologically acceptable carriers, diluents, and/or excipients; and ii) one or more compounds provided herein.

In one aspect, provided herein are methods for treating a patient by administering a compound provided herein. In some embodiments, provided herein is a method of inhibiting the activity of tyrsoine kinase(s), such as Btk, or of treating a disease, disorder, or condition, which would benefit from inhibition of tyrosine kinase(s), such as Btk, in a patient, which includes administering to the patient a therapeutically effective amount of at least one of any of the compounds herein, or pharmaceutically acceptable salt, or stereoisomer thereof.

In another aspect, provided herein is the use of a compound disclosed herein for inhibiting Bruton's tyrosine kinase (Btk) activity or for the treatment of a disease, disorder, or condition, which would benefit from inhibition of Bruton's tyrosine kinase (Btk) activity.

In some embodiments, compounds provided herein are administered to a human.

In some embodiments, compounds provided herein are orally administered.

In some embodiments, compounds provided herein are used for the formulation of a medicament for the inhibition of tyrosine kinase activity. In some other embodiments, compounds provided herein are used for the formulation of a medicament for the inhibition of Bruton's tyrosine kinase (Btk) activity.

Articles of manufacture including packaging material, a compound or composition or pharmaceutically acceptable derivative thereof provided herein, which is effective for inhibiting the activity of tyrosine kinase(s), such as Btk, within the packaging material, and a label that indicates that the compound or composition, or pharmaceutically acceptable salt, or stereoisomer thereof, is used for inhibiting the activity of tyrosine kinase(s), such as Btk, are provided.

In a further aspect, provided herein is a method for inhibiting Bruton's tyrosine kinase in a subject in need thereof by administering to the subject thereof a composition containing a therapeutically effective amount of at least one compound having the structure of Formula (I), (II), (III), (IIIa), (IVa)-(IVd), (Va)-(Vd), (VIa)-(VId) or (VIIa)-(VIId).

In some embodiments, the subject in need is suffering from an autoimmune disease, e.g., inflammatory bowel disease, arthritis, lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behçet's disease, chronic fatigue, dysautonomia, endometriosis, interstitial cystitis, neuromyotonia, scleroderma, or vulvodynia.

In some embodiments, the subject in need is suffering from a heteroimmune condition or disease, e.g., graft versus host disease, transplantation, transfusion, anaphylaxis, allergy, type I hypersensitivity, allergic conjunctivitis, allergic rhinitis, or atopic dermatitis.

In some embodiments, the subject in need is suffering from an inflammatory disease, e.g., asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In some embodiments, the subject in need is suffering from a cancer. In one embodiment, the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis. In some embodiments, where the subject is suffering from a cancer, an anti-cancer agent is administered to the subject in addition to one of the above-mentioned compounds. In one embodiment, the anti-cancer agent is an inhibitor of mitogen-activated protein kinase signaling, e.g., U0126, PD98059, PD184352, PD0325901, ARRY-142886, SB239063, SP600125, BAY 43-9006, wortmannin, or LY294002.

In some embodiments, the subject in need is suffering from a thromboembolic disorder, e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

In another aspect, provided herein is a method for treating an autoimmune disease by administering to a subject in need thereof a composition containing a therapeutically effective amount of at least one compound having the structure of Formula (I), (II), (III), (IIIa), (IVa)-(IVd), (Va)-(Vd), (VIa)-(VId) or (VIIa)-(VIId). In some embodiments, the autoimmune disease is arthritis. In some embodiments, the autoimmune disease is lupus. In some embodiments, the autoimmune disease is inflammatory bowel disease (such as Crohn's disease or ulcerative colitis), rheumatoid arthritis, psoriatic arthritis, osteoarthritis, Still's disease, juvenile arthritis, lupus, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behçet's disease, chronic fatigue, dysautonomia, endometriosis, interstitial cystitis, neuromyotonia, scleroderma, or vulvodynia.

In another aspect, provided herein is a method for treating a heteroimmune condition or disease by administering to a subject in need thereof a composition containing a therapeutically effective amount of at least one compound having the structure of Formula (I), (II), (III), (IIIa), (IVa)-(IVd), (Va)-(Vd), (VIa)-(VId) or (VIIa)-(VIId). In some embodiments, the heteroimmune condition or disease is graft versus host disease, transplantation, transfusion, anaphylaxis, allergy, type I hypersensitivity, allergic conjunctivitis, allergic rhinitis, or atopic dermatitis.

In another aspect, provided herein is a method for treating an inflammatory disease by administering to a subject in need thereof a composition containing a therapeutically effective amount of at least one compound having the structure of Formula (I), (II), (III), (IIIa), (IVa)-(IVd), (Va)-(Vd), (VIa)-(VId) or (VIIa)-(VIId). In some embodiments, the inflammatory disease is asthma, inflammatory bowel disease (such as Crohn's disease or ulcerative colitis), appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In yet another aspect, provided herein is a method for treating a cancer by administering to a subject in need thereof a composition containing a therapeutically effective amount of at least one compound having the structure of Formula (I), (II), (III), (IIIa), (IVa)-(IVd), (Va)-(Vd), (VIa)-(VId) or (VIIa)-(VIId). In some embodiments, the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis. In some embodiments, where the subject is suffering from a cancer, an anti-cancer agent is administered to the subject in addition to one of the above-mentioned compounds. In some embodiments, the anti-cancer agent is an inhibitor of mitogen-activated protein kinase signaling, e.g., U0126, PD98059, PD184352, PD0325901, ARRY-142886, SB239063, SP600125, BAY 43-9006, wortmannin, or LY294002.

In another aspect, provided herein is a method for treating a thromboembolic disorder by administering to a subject in need thereof a composition containing a therapeutically effective amount of at least one compound having the structure of Formula (I), (II), (III), (IIIa), (IVa)-(IVd), (Va)-(Vd), (VIa)-(VId) or (VIIa)-(VIId). In some embodiments, the thromboembolic disorder is myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

In another aspect, provided herein is a method for treating an autoimmune disease by administering to a subject in need thereof a composition containing a therapeutically effective amount of a compound that forms a covalent bond with Bruton's tyrosine kinase. In some embodiments, the compound forms a covalent bond with the activated form of Bruton's tyrosine kinase. In some embodiments, the compound irreversibly inhibits the Bruton's tyrosine kinase to which it is covalently bound. In some embodiments, the compound forms a covalent bond with a cysteine residue on Bruton's tyrosine kinase.

In another aspect, provided herein is a method for treating a heteroimmune condition or disease by administering to a subject in need thereof a composition containing a therapeutically effective amount of a compound that forms a covalent bond with Bruton's tyrosine kinase. In some embodiments, the compound forms a covalent bond with the activated form of Bruton's tyrosine kinase. In some embodiments, the compound irreversibly inhibits the Bruton's tyrosine kinase to which it is covalently bound. In some embodiments, the compound forms a covalent bond with a cysteine residue on Bruton's tyrosine kinase.

In another aspect, provided herein is a method for treating an inflammatory disease by administering to a subject in need thereof a composition containing a therapeutically effective amount of a compound that forms a covalent bond with Bruton's tyrosine kinase. In some embodiments, the compound forms a covalent bond with the activated form of Bruton's tyrosine kinase. In some embodiments, the compound irreversibly inhibits the Bruton's tyrosine kinase to which it is covalently bound. In some embodiments, the compound forms a covalent bond with a cysteine residue on Bruton's tyrosine kinase.

In another aspect, provided herein is a method for treating a cancer by administering to a subject in need thereof a composition containing a therapeutically effective amount of a compound that forms a covalent bond with Bruton's tyrosine kinase. In some embodiments, the compound forms a covalent bond with the activated form of Bruton's tyrosine kinase. In some embodiments, the compound irreversibly inhibits the Bruton's tyrosine kinase to which it is covalently bound. In some embodiments, the compound forms a covalent bond with a cysteine residue on Bruton's tyrosine kinase.

In another aspect, provided herein is a method for treating a thromboembolic disorder by administering to a subject in need thereof a composition containing a therapeutically effective amount of a compound that forms a covalent bond with Bruton's tyrosine kinase. In some embodiments, the compound forms a covalent bond with the activated form of Bruton's tyrosine kinase. In some embodiments, the compound irreversibly inhibits the Bruton's tyrosine kinase to which it is covalently bound. In some embodiments, the compound forms a covalent bond with a cysteine residue on Bruton's tyrosine kinase.

In another aspect the present invention provides methods for modulating, including irreversibly inhibiting, the activity of Btk or other tyrosine kinases, wherein the other tyrosine kinases share homology with Btk by having a cysteine residue (including a Cys 481 residue) that can form a covalent bond with at least one irreversible inhibitor described herein, in a subject.

The methods comprise administering to the subject at least once an effective amount of at least one compound having the structure of Formula (I), (II), (III), (IIIa), (IVa)-(IVd), (Va)-(Vd), (VIa)-(VId) or (VIIa)-(VIId). In another aspect the present invention provides methods for modulating, including reversibly or irreversibly inhibiting, the activity of Btk in a subject comprising administering to the subject at least once an effective amount of at least one compound having the structure of Formula (I), (II), (III), (IIIa), (IVa)-(IVd), (Va)-(Vd), (VIa)-(VId) or (VIIa)-(VIId). In another aspect the present invention provides methods for treating Btk-dependent or Btk mediated conditions or diseases, comprising administering to the subject at least once an effective amount of at least one compound having the structure of (I), (II), (III), (IIIa), (IVa)-(IVd), (Va)-(Vd), (VIa)-(VId) or (VIIa)-(VIId).

In another aspect the present invention provides methods for treating inflammation comprising administering to the subject at least once an effective amount of at least one compound having the structure of Formula (I), (II), (III), (IIIa), (IVa)-(IVd), (Va)-(Vd), (VIa)-(VId) or (VIIa)-(VIId).

In another aspect the present invention provides methods for the treatment of cancer comprising administering to the subject at least once an effective amount of at least one compound having the structure of Formula (I), (II), (III), (IIIa), (IVa)-(IVd), (Va)-(Vd), (VIa)-(VId) or (VIIa)-(VIId). The type of cancer may include, but is not limited to, pancreatic cancer and other solid or hematological tumors. In some embodiments, the cancer is one of the cancers disclosed herein.

In another aspect the present invention provides methods for treating respiratory diseases comprising administering to the subject at least once an effective amount of at least one compound having the structure of Formula (I), (II), (III), (IIIa), (IVa)-(IVd), (Va)-(Vd), (VIa)-(VId) or (VIIa)-(VIId). In some embodiments, the respiratory disease is asthma. In some embodiments, the respiratory disease includes, but is not limited to, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, and seasonal asthma.

In another aspect the present invention provides methods for treating rheumatoid arthritis and osteoarthritis comprising administering to the subject at least once an effective amount of at least one compound having the structure of Formula (I), (II), (III), (IIIa), (IVa)-(IVd), (Va)-(Vd), (VIa)-(VId) or (VIIa)-(VIId).

In another aspect the present invention provides methods for treating inflammatory responses of the skin comprising administering to the subject at least once an effective amount of at least one compound having the structure of Formula (I), (II), (III), (IIIa), (IVa)-(IVd), (Va)-(Vd), (VIa)-(VId) or (VIIa)-(VIId). Such inflammatory responses of the skin include, by way of example, dermatitis, contact dermatitis, eczema, urticaria, rosacea, and scarring.

In another aspect the present invention provides methods for reducing psoriatic lesions in the skin, joints, or other tissues or organs, comprising administering to the subject an effective amount of a first compound having the structure of Formula (I), (II), (III), (IIIa), (IVa)-(IVd), (Va)-(Vd), (VIa)-(VId) or (VIIa)-(VIId).

In another aspect the present invention is the use of a compound of Formula (I), (II), (III), (IIIa), (IVa)-(IVd), (Va)-(Vd), (VIa)-(VId) or (VIIa)-(VIId) in the manufacture of a medicament for treating an inflammatory disease or condition in an animal in which the activity of Btk or other tyrosine kinases, wherein the other tyrosine kinases share homology with Btk by having a cysteine residue (including a Cys 481 residue) that can form a covalent bond with at least one irreversible inhibitor described herein, contributes to the pathology and/or symptoms of the disease or condition. In one embodiment of this aspect, the tyrosine kinase protein is Btk. In some embodiments, the inflammatory disease or conditions are respiratory, cardiovascular, or proliferative diseases.

In embodiments of any of the aforementioned aspects, the compounds disclosed herein may be administered enterally, parenterally, or both. In some embodiments, (a) the effective amount of the compound is systemically administered to the subject; (b) the effective amount of the compound is administered orally to the subject; (c) the effective amount of the compound is intravenously administered to the subject; (d) the effective amount of the compound administered by inhalation; (e) the effective amount of the compound is administered by nasal administration; or (f) the effective amount of the compound is administered by injection to the subject; (g) the effective amount of the compound is administered topically (dermal) to the subject; (h) the effective amount of the compound is administered by ophthalmic administration; or (i) the effective amount of the compound is administered rectally to the subject.

In embodiments of any of the aforementioned aspects, the compound may be administered in a single administration of the effective amount of the compound. In some embodiments, (i) the compound is administered to the subject once; (ii) the compound is administered to the subject multiple times over the span of one day; (iii) the compound is administered to the subject continually; or (iv) the compound is administered to the subject continuously.

In embodiments of any of the aforementioned aspects, the compound may be administered in multiple administrations that, taken together, comprise an effective amount of the compound. In some embodiments (i) the time between administrations is at least 6 hours; or (ii) the time between multiple administrations is every 8 hours. In further or alternative embodiments, multiple administrations include a drug holiday, wherein the administration of the compound is temporarily suspended or the dose of the compound being administered is temporarily reduced; at the end of the drug holiday, dosing of the compound is resumed. The length of the drug holiday can vary from 2 days to 1 year.

In embodiments of any of the aforementioned aspects involving the treatment of proliferative disorders, including cancer, at least one additional agent is administered. In some embodiments, the additional agent is selected from alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzumab, methotrexate, Paclitaxel™, taxol, temozolomide, thioguanine, or classes of drugs including hormones (an antiestrogen, an antiandrogen, or gonadotropin releasing hormone analogues), interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, dronabinol.

In embodiments of any of the aforementioned aspects involving the treatment of Btk-dependent or tyrosine kinase mediated diseases or conditions, patients are identified by screening for a tyrosine kinase gene haplotype. In some embodiments the tyrosine kinase gene haplotype is a tyrosine kinase pathway gene. In some embodiments, the tyrosine kinase gene haplotype is a Btk haplotype.

In some embodiments, the compounds of Formula (I), (II), (III), (IIIa), (IVa)-(IVd), (Va)-(Vd), (VIa)-(VId) or (VIIa)-(VIId) are reversible inhibitors of Bruton's tyrosine kinase (Btk). In some embodiments, such reversible inhibitors are selective for Btk. In some embodiments, such reversible inhibitors have an IC₅₀ below about 10 μM in enzyme assay. In some embodiments embodiment, the reversible inhibitors have an IC₅₀ of less than about 1 μM, preferably less than about 0.25 μM.

In some embodiments, the compounds of Formula (I), (II), (III), (IIIa), (IVa)-(IVd), (Va)-(Vd), (VIa)-(VId) or (VIIa)-(VIId) are selective reversible inhibitors for Btk over Itk. In some embodiments, the compounds of Formula (I), (II), (III), (IIIa), (IVa)-(IVd), (Va)-(Vd), (VIa)-(VId) or (VIIa)-(VIId) are selective reversible inhibitors for Btk over Lck. In some embodiments, the compounds of Formula (I), (II), (III), (IIIa), (IVa)-(IVd), (Va)-(Vd), (VIa)-(VId) or (VIIa)-(VIId) are selective reversible inhibitors for Btk over ABL. In some embodiments, the compounds of Formula (I), (II), (III), (IIIa), (IVa)-(IVd), (Va)-(Vd), (VIa)-(VId) or (VIIa)-(VIId) are selective reversible inhibitors for Btk over CMET. In some embodiments, the compounds of Formula (I), (II), (III), (IIIa), (IVa)-(IVd), (Va)-(Vd), (VIa)-(VId) or (VIIa)-(VIId) are selective reversible inhibitors for Btk over EGFR. In some embodiments, the compounds of Formula (I), (II), (III), (IIIa), (IVa)-(IVd), (Va)-(Vd), (VIa)-(VId) or (VIIa)-(VIId) are selective reversible inhibitors for Btk over Lyn.

In some embodiments, the reversible Btk inhibitors are also inhibitors of EGFR.

In some embodiments, the compounds of Formula (I), (II), (III), (IIIa), (IVa)-(IVd), (Va)-(Vd), (VIa)-(VId) or (VIIa)-(VIId) are irreversible inhibitors of Bruton's tyrosine kinase (Btk). In some embodiments, such irreversible inhibitors are selective for Btk. In some embodiments, such inhibitors have an IC₅₀ below about 10 μM in enzyme assay. In some embodiments, such Btk irreversible inhibitors have an IC₅₀ of less than about 1 μM, preferably less than about 0.25 μM.

In some embodiments, the compounds of Formula (I), (II), (III), (IIIa), (IVa)-(IVd), (Va)-(Vd), (VIa)-(VId) or (VIIa)-(VIId) are selective irreversible inhibitors for Btk over Itk. In some embodiments, the compounds of Formula (I), (II), (III), (IIIa), (IVa)-(IVd), (Va)-(Vd), (VIa)-(VId) or (VIIa)-(VIId) are selective irreversible inhibitors for Btk over Lck. In some embodiments, the compounds of Formula (I), (II), (III), (IIIa), (IVa)-(IVd), (Va)-(Vd), (VIa)-(VId) or (VIIa)-(VIId) are selective irreversible inhibitors for Btk over ABL. In some embodiments, the compounds of Formula (I), (II), (III), (IIIa), (IVa)-(IVd), (Va)-(Vd), (VIa)-(VId) or (VIIa)-(VIId) are selective irreversible inhibitors for Btk over CMET. In some embodiments, the compounds of Formula (I), (II), (III), (IIIa), (IVa)-(IVd), (Va)-(Vd), (VIa)-(VId) or (VIIa)-(VIId) are selective irreversible inhibitors for Btk over EGFR. In some embodiments, the compounds of Formula (I), (II), (III), (IIIa), (IVa)-(IVd), (Va)-(Vd), (VIa)-(VId) or (VIIa)-(VIId) are selective irreversible inhibitors for Btk over Lyn.

In some embodiments, the irreversible Btk inhibitors are also inhibitors of EGFR.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description. The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

### DETAILED DESCRIPTION OF THE INVENTION

### Certain Terminology

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Definitions of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl (n-pr), 1-methylethyl (iso-propyl or i-Pr), n-butyl (n-Bu), n-pentyl, 1,1-dimethylethyl (t-butyl or t-Bu), 3-methylhexyl, 2-methylhexyl, and the like. Unless stated otherwise specifically in the specification, an alkyl group is optionally substituted by one or more of the following substituents: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

The alkyl group could also be a “lower alkyl” having 1 to 6 carbon atoms.

As used herein, C₁-Cₓ includes C₁-C₂, C₁-C₃ . . . C₁-Cₓ.

“Alkenyl” refers to a straight or branched hydrocarbon chain radical group consisting solely of carbon and hydrogen atoms, containing at least one double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkynyl” refers to a straight or branched hydrocarbon chain radical group consisting solely of carbon and hydrogen atoms, containing at least one triple bond, having from two to twelve carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. Unless stated otherwise specifically in the specification, an alkylene chain is optionally substituted by one or more of the following substituents: halo, cyano, nitro, aryl, cycloalkyl, heterocyclyl, heteroaryl, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, for example, ethenylene, propenylene, n-butenylene, and the like. The alkenylene chain is attached to the rest of the molecule through a double bond or a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain is optionally substituted by one or more of the following substituents: halo, cyano, nitro, aryl, cycloalkyl, heterocyclyl, heteroaryl, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, and where each of the above substituents is unsubstituted unless otherwise indicated.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hickel theory. Aryl groups include, but are not limited to, groups such as phenyl (Ph), fluorenyl, and naphthyl. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, benzyl, diphenylmethyl and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is optionally saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—SR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Halo” or “halogen” refers to bromo, chloro, fluoro or iodo substituents.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical is optionally substituted as defined above for an alkyl group.

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three to 14 ring atoms, such as three, four, five, six, seven, eight, nine, or more than nine ring atoms. Cₓheterocycloalkyl refers to a heterocycloalkyl having x number of ring carbon atoms wherein the remaining ring atom(s) are heteroatom(s). Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

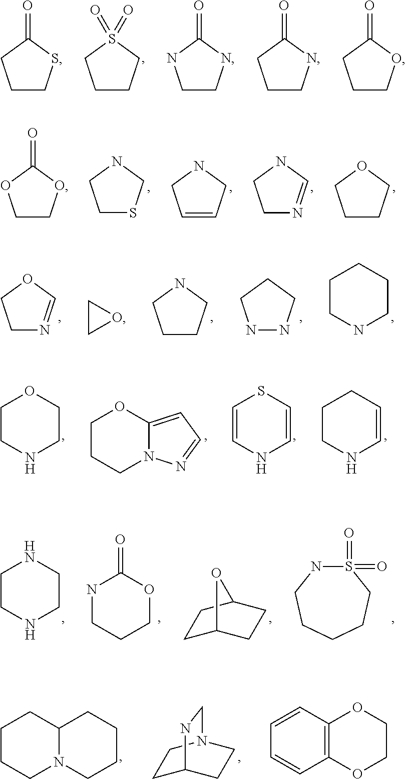

and the like. The term heteroalicyclic also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group).

“Heteroaryl” refers to a radical derived from a 3- to 18-membered aromatic ring radical that comprises at least one heteroatom, in particular, one to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system contains a heteroatom and is fully unsaturated, i.e., it contains a cyclic, delocalized (4n+2) π-electron system in accordance with the Hickel theory. Heteroaryl includes fused or bridged ring systems. In some embodiments, heteroaryl rings have five, six, seven, eight, nine, or more than nine ring atoms. Cₓheteroaryl refers to a heteroaryl having x number of ring carbon atoms wherein the remaining ring atom(s) are heteroatom(s). The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolin yl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—SR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Sulfanyl” refers to the —S— radical.

“Sulfinyl” refers to the —S(═O)— radical.

“Sulfonyl” refers to the —S(═O)₂— radical.

“Cyano” refers to the —CN radical.

“Nitro” refers to the —NO₂ radical.

“Oxa” refers to the —O— radical.

“Oxo” refers to the ═O radical.

“Imino” refers to the ═NH radical.

“Thioxo” refers to the ═S radical.

An “alkoxy” group refers to an (alkyl)O— group, where alkyl is as defined herein.

An “aryloxy” group refers to an (aryl)O— group, where aryl is as defined herein.

“Carbocyclylalkyl” means an alkyl radical, as defined herein, substituted with a carbocyclyl group. “Cycloalkylalkyl” means an alkyl radical, as defined herein, substituted with a cycloalkyl group. Non-limiting cycloalkylalkyl groups include cyclopropylmethyl, cyclobutylmethyl, cyclopentylmethyl, cyclohexylmethyl, and the like.

As used herein, the terms “heteroalkyl,” “heteroalkenyl” and “heteroalkynyl” include optionally substituted alkyl, alkenyl and alkynyl radicals in which one or more (e.g., 1, 2 or 3) skeletal chain atoms is a heteroatom, e.g., oxygen, nitrogen, sulfur, silicon, phosphorus or combinations thereof. The heteroatom(s) may be placed at any interior position of the heteroalkyl group or at the position at which the heteroalkyl group is attached to the remainder of the molecule. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The term “bond,” “direct bond” or “single bond” refers to a chemical bond between two atoms, or two moieties when the atoms joined by the bond are considered to be part of larger sub structure.

An “isocyanato” group refers to a —NCO group.

An “isothiocyanato” group refers to a —NCS group.

The term “moiety” refers to a specific segment or functional group of a molecule. Chemical moieties are often recognized chemical entities embedded in or appended to a molecule.

A “thioalkoxy” or “alkylthio” group refers to a —S-alkyl group.

An “alkylthioalkyl” group refers to an alkyl group substituted with a —S-alkyl group.

As used herein, the term “acyloxy” refers to a group of formula RC(═O)O—.

“Carboxy” means a —C(O)OH radical.

As used herein, the term “acetyl” refers to a group of formula —C(═O)CH₃.

As used herein, the term “Acyl” refers to the group —C(O)R.

As used herein, the term “trihalomethanesulfonyl” refers to a group of formula X₃CS(═O)₂— where X is a halogen.

“Cyanoalkyl” means an alkyl radical, as defined herein, substituted with at least one cyano group.

As used herein, the term “N-sulfonamido” or “sulfonylamino” refers to a group of formula RS(═O)₂NH—.

As used herein, the term “O-carbamyl” refers to a group of formula —OC(═O)NR₂.

As used herein, the term “N-carbamyl” refers to a group of formula ROC(═O)NH—.

As used herein, the term “O-thiocarbamyl” refers to a group of formula —OC(═S)NR₂.

As used herein, “N-thiocarbamyl” refers to a group of formula ROC(═S)NH—.

As used herein, the term “C-amido” refers to a group of formula —C(═O)NR₂.

“Aminocarbonyl” refers to a —CONH₂ radical.

As used herein, the term “N-amido” refers to a group of formula RC(═O)NH—.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

“Hydroxyalkyl” refers to an alkyl radical, as defined herein, substituted with at least one hydroxy group. Non-limiting examples of a hydroxyalkyl include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl.

“Alkoxyalkyl” refers to an alkyl radical, as defined herein, substituted with an alkoxy group, as defined herein.

An “alkenyloxy” group refers to an (alkenyl)O— group, where alkenyl is as defined herein.

An “amine” or “amino” is a chemical moiety with the formula refer to both unsubstituted and substituted amines and salts thereof, e.g., a moiety that can be represented by

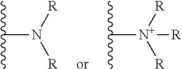

wherein each R independently represents a hydrogen or an alkyl, cycloalkyl, aryl, heteroaryl (bonded through a ring carbon) or heteroalicyclic (bonded through a ring carbon) group, or two R are taken together with the N atom to which they are attached complete a heterocycle having from 4 to 8 atoms in the ring structure.

The term “alkylamine” or “alkylamino” refers to the —N(alkyl)ₓH_{y} group, where x and y are selected from among x=1, y=1 and x=2, y=0. When x=2, the alkyl groups, taken together with the N atom to which they are attached, can optionally form a cyclic ring system.

“Alkylaminoalkyl” refers to an alkyl radical, as defined herein, substituted with an alkylamine, as defined herein.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon).

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, acyl, nitro, haloalkyl, fluoroalkyl, haloalkoxy, amino, including mono- and di-substituted amino groups, and the N-oxide and protected derivatives thereof; or “optionally substituted” or “substituted” may be -L^{s}R^{s}, wherein each L^{s} is independently selected from a bond, —O—, —C(═O)—, —S—, —S(═O)—, —S(═O)₂—, —NH—, —N(CH₃)—, —NHC(O)—, —N(CH₃)C(O)—, —C(O)NH—, —C(O)N(CH₃)—, S(═O)₂NH—, —NHS(═O)₂—, —OC(O)NH—, —NHC(O)O—, -(substituted or unsubstituted C₁-C₆ alkyl)-, or -(substituted or unsubstituted C₂-C₆ alkenyl)-; and each R^{s} is independently selected from H, C₁-C₆alkyl, C₂-C₆ alkenyl, C₃-C₈cycloalkyl, C₂-C₇heterocycloalkyl, C₆-C₁₂aryl, C₁-C₁₂heteroaryl, or C₁-C₆heteroalkyl. The protecting groups that may form the protective derivatives of the above substituents are known to those of skill in the art and may be found in references such as Green and Wuts, PROTECTIVE GROUPS IN ORGANIC SYNTHESIS 3^{rd} Ed., (Wiley 1999).

Provided herein are various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or“trans,” where“cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

“Enantiomers” are a pair of stereoisomers that are non-superimposable mirror images of each other. A mixture of a pair of enantiomers in any proportion can be known as a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is an enantiomer, the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry at each asymmetric atom, as (R)- or (S)-. The present chemical entities, pharmaceutical compositions and methods are meant to include all such possible isomers, including racemic mixtures, optically substantially pure forms and intermediate mixtures. Optically active (R)- and (S)-isomers can be prepared, for example, using chiral synthons or chiral reagents, or resolved using conventional techniques.

The “enantiomeric excess” or “% enantiomeric excess” of a composition can be calculated using the equation shown below. In the example shown below, a composition contains 90% of one enantiomer, e.g., the S enantiomer, and 10% of the other enantiomer, e.g., the R enantiomer.

ee=(90−10)/100=80%.

Thus, a composition containing 90% of one enantiomer and 10% of the other enantiomer is said to have an enantiomeric excess of 80%. Some compositions described herein contain an enantiomeric excess of at least about 50%, about 75%, about 90%, about 95%, or about 99% of the S enantiomer. In other words, the compositions contain an enantiomeric excess of the S enantiomer over the R enantiomer. In other embodiments, some compositions described herein contain an enantiomeric excess of at least about 50%, about 75%, about 90%, about 95%, or about 99% of the R enantiomer. In other words, the compositions contain an enantiomeric excess of the R enantiomer over the S enantiomer.

For instance, an isomer/enantiomer can, in some embodiments, be provided substantially free of the corresponding enantiomer, and can also be referred to as “optically enriched,” “enantiomerically enriched,” “enantiomerically pure” and “non-racemic,” as used interchangeably herein. These terms refer to compositions in which the percent by weight of one enantiomer is greater than the amount of that one enantiomer in a control mixture of the racemic composition (e.g., greater than 1:1 by weight). For example, an enantiomerically enriched preparation of the S enantiomer means a preparation of the compound having greater than about 50% by weight of the S enantiomer relative to the R enantiomer, such as at least about 75% by weight, further such as at least about 80% by weight. In some embodiments, the enrichment can be much greater than about 80% by weight, providing a “substantially enantiomerically enriched,” “substantially enantiomerically pure” or a “substantially non-racemic” preparation, which refers to preparations of compositions which have at least about 85% by weight of one enantiomer relative to other enantiomer, such as at least about 90% by weight, and further such as at least about 95% by weight. In certain embodiments, the compound provided herein is made up of at least about 90% by weight of one enantiomer. In other embodiments, the compound is made up of at least about 95%, about 98%, or about 99% by weight of one enantiomer.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more. In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

In other embodiments, the compound mixture contains identical chemical entities except for their stereochemical orientations, namely (S)- or (R)-isomers. For example, if a compound disclosed herein has a —CH(R)— unit, and R is not hydrogen, then the —CH(R)— is in an (S)- or (R)-stereochemical orientation for each of the identical chemical entities. In some embodiments, the mixture of identical chemical entities is a racemic mixture of (S)- and (R)-isomers. In another embodiment, the mixture of the identical chemical entities (except for their stereochemical orientations), contain predominately (S)-isomers or predominately (R)-isomers. For example, the (S)-isomers in the mixture of identical chemical entities are present at about 55%, about 60%, about 65%, about 70%, about 75%, about 80%, about 85%, about 90%, about 95%, about 96%, about 97%, about 98%, about 99%, about 99.5%, or more, relative to the (R)-isomers. In some embodiments, the (S)-isomers in the mixture of identical chemical entities are present at an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

In another embodiment, the (R)-isomers in the mixture of identical chemical entities (except for their stereochemical orientations), are present at about 55%, about 60%, about 65%, about 70%, about 75%, about 80%, about 85%, about 90%, about 95%, about 96%, about 97%, about 98%, about 99%, about 99.5%, or more, relative to the (S)-isomers. In some embodiments, the (R)-isomers in the mixture of identical chemical entities (except for their stereochemical orientations), are present at a (R)-enantiomeric excess greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

Enantiomers can be isolated from racemic mixtures by any method known to those skilled in the art, including chiral high pressure liquid chromatography (HPLC), the formation and crystallization of chiral salts, or prepared by asymmetric syntheses. See, for example, Enantiomers, Racemates and Resolutions (Jacques, Ed., Wiley Interscience, New York, 1981); Wilen et al., Tetrahedron 33:2725 (1977); Stereochemistry of Carbon Compounds (E. L. Eliel, Ed., McGraw-Hill, N Y, 1962); and Tables of Resolving Agents and Optical Resolutions p. 268 (E. L. ElM, Ed., Univ. of Notre Dame Press, Notre Dame, Ind. 1972).

Optical isomers can be obtained by resolution of the racemic mixtures according to conventional processes, e.g., by formation of diastereoisomeric salts, by treatment with an optically active acid or base. Examples of appropriate acids are tartaric, diacetyltartaric, dibenzoyltartaric, ditoluoyltartaric, and camphorsulfonic acid. The separation of the mixture of diastereoisomers by crystallization followed by liberation of the optically active bases from these salts affords separation of the isomers. Another method involves synthesis of covalent diastereoisomeric molecules by reacting disclosed compounds with an optically pure acid in an activated form or an optically pure isocyanate. The synthesized diastereoisomers can be separated by conventional means such as chromatography, distillation, crystallization or sublimation, and then hydrolyzed to deliver the enantiomerically enriched compound. Optically active compounds can also be obtained by using active starting materials. In some embodiments, these isomers can be in the form of a free acid, a free base, an ester or a salt.

In certain embodiments, the pharmaceutically acceptable form is a tautomer. As used herein, the term “tautomer” is a type of isomer that includes two or more interconvertible compounds resulting from at least one formal migration of a hydrogen atom and at least one change in valency (e.g., a single bond to a double bond, a triple bond to a single bond, or vice versa). “Tautomerization” includes prototropic or proton-shift tautomerization, which is considered a subset of acid-base chemistry. “Prototropic tautomerization” or “proton-shift tautomerization” involves the migration of a proton accompanied by changes in bond order. The exact ratio of the tautomers depends on several factors, including temperature, solvent, and pH. Where tautomerization is possible (e.g., in solution), a chemical equilibrium of tautomers can be reached. Tautomerizations (i.e., the reaction providing a tautomeric pair) can be catalyzed by acid or base, or can occur without the action or presence of an external agent. Exemplary tautomerizations include, but are not limited to, keto-to-enol; amide-to-imide; lactam-to-lactim; enamine-to-imine; and enamine-to-(a different) enamine tautomerizations. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

In certain embodiments, the therapeutic preparation may be enriched to provide predominantly one diastereomer of a compound (e.g., of Formula (I)). A diastereomerically enriched mixture may comprise, for example, at least about 60 mol percent of one diastereomer, or more preferably at least about 75, about 90, about 95, or even about 99 mol percent.

The term “nucleophile” or “nucleophilic” refers to an electron rich compound, or moiety thereof. An example of a nucleophile includes, but in no way is limited to, a cysteine residue of a molecule, such as, for example Cys 481 of Btk.

The term “electrophile,” or “electrophilic” refers to an electron poor or electron deficient molecule, or moiety thereof. Examples of electrophiles include, but in no way are limited to, Michael acceptor moieties.

The term “acceptable” or “pharmaceutically acceptable,” with respect to a formulation, composition, excipient, diluent, or ingredient, as used herein, means having no persistent detrimental effect on the general health of the subject being treated or does not abrogate the biological activity or properties of the compound, and is relatively nontoxic.

As used herein, the term “agonist” refers to a compound, the presence of which results in a biological activity of a protein that is the same as the biological activity resulting from the presence of a naturally occurring ligand for the protein, such as, for example, Btk.

As used herein, “amelioration” of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The term “Bruton's tyrosine kinase,” as used herein, refers to Bruton's tyrosine kinase from Homo sapiens, as disclosed in, e.g., U.S. Pat. No. 6,326,469 (GenBank Accession No. NP_000052).

The term “Bruton's tyrosine kinase homolog,” as used herein, refers to orthologs of Bruton's tyrosine kinase, e.g., the orthologs from mouse (GenBank Acession No. AAB47246), dog (GenBank Acession No. XP_549139.), rat (GenBank Acession No. NP_001007799), chicken (GenBank Acession No. NP_989564), or zebra fish (GenBank Acession No. XP_698117), and fusion proteins of any of the foregoing that exhibit kinase activity towards one or more substrates of Bruton's tyrosine kinase (e.g. a peptide substrate having the amino acid sequence “AVLESEEELYSSARQ”).

The terms “co-administration” or the like, as used herein, are meant to encompass administration of the selected therapeutic agents to a single patient, and are intended to include treatment regimens in which the agents are administered by the same or different route of administration or at the same or different time. In some embodiments, the term “co-administration” or the like, is meant to encompass the administration of the selected therapeutic agents in the same cycle(s). In these embodiments, the selected therapeutic agents may be administered on the same or different days of the cycle(s).

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound of any of Formula (I), (II), (III), (IIIa), (IV), (V) or (VI), age, weight, general condition of the subject, the condition being treated, the severity of the condition being treated, and the judgment of the prescribing physician.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the treating physician.

The term “homologous cysteine,” as used herein refers to a cysteine residue found with in a sequence position that is homologous to that of cysteine 481 of Bruton's tyrosine kinase, as defined herein. For example, cysteine 482 is the homologous cysteine of the rat ortholog of Bruton's tyrosine kinase; cysteine 479 is the homologous cysteine of the chicken ortholog; and cysteine 481 is the homologous cysteine in the zebra fish ortholog. In another example, the homologous cysteine of TXK, a Tec kinase family member related to Bruton's tyrosine, is Cys 350.

The terms “inhibits,” “inhibiting” or “inhibitor” of a kinase, as used herein, refer to inhibition of enzymatic phosphotransferase activity.

The term “irreversible inhibitor,” as used herein, refers to a compound that, upon contact with a target protein (e.g., a kinase) causes the formation of a new covalent bond with or within the protein, whereby one or more of the target protein's biological activities (e.g., phosphotransferase activity) is diminished or abolished notwithstanding the subsequent presence or absence of the irreversible inhibitor. In contrast, a reversible inhibitor compound upon contact with a target protein does not cause the formation of a new covalent bond with or within the protein and therefore can associate and dissociate from the target protein.

The term “irreversible Btk inhibitor,” as used herein, refers to an inhibitor of Btk that can form a covalent bond with an amino acid residue of Btk. In one embodiment, the irreversible inhibitor of Btk can form a covalent bond with a Cys residue of Btk; in particular embodiments, the irreversible inhibitor can form a covalent bond with a Cys 481 residue (or a homolog thereof) of Btk or a cysteine residue in the homologous corresponding position of another tyrosine kinase.

The term “isolated,” as used herein, refers to separating and removing a component of interest from components not of interest. Isolated substances can be in either a dry or semi-dry state, or in solution, including but not limited to an aqueous solution. The isolated component can be in a homogeneous state or the isolated component can be a part of a pharmaceutical composition that comprises additional pharmaceutically acceptable carriers and/or excipients. By way of example only, nucleic acids or proteins are “isolated” when such nucleic acids or proteins are free of at least some of the cellular components with which it is associated in the natural state, or that the nucleic acid or protein has been concentrated to a level greater than the concentration of its in vivo or in vitro production. Also, by way of example, a gene is isolated when separated from open reading frames which flank the gene and encode a protein other than the gene of interest.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “prophylactically effective amount,” as used herein, refers that amount of a composition applied to a patient which will relieve to some extent one or more of the symptoms of a disease, condition or disorder being treated. In such prophylactic applications, such amounts may depend on the patient's state of health, weight, and the like.

As used herein, the term “selective binding compound” refers to a compound that selectively binds to any portion of one or more target proteins.

As used herein, the term “selectively binds” refers to the ability of a selective binding compound to bind to a target protein, such as, for example, Btk, with greater affinity than it binds to a non-target protein. In certain embodiments, specific binding refers to binding to a target with an affinity that is at least about 10, about 50, about 100, about 250, about 500, about 1000 or more times greater than the affinity for a non-target.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater.

The term “subject” or “patient” as used herein, to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, the term “target protein” refers to a molecule or a portion of a protein capable of being bound by a selective binding compound. In certain embodiments, a target protein is Btk.

The terms “treat,” “treating” or “treatment”, as used herein, include alleviating, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, the term IC₅₀ refers to an amount, concentration or dosage of a particular test compound that achieves a 50% inhibition of a maximal response, such as inhibition of Btk, in an assay that measures such response.

As used herein, the term EC₅₀ refers to a dosage, concentration or amount of a particular test compound that elicits a dose-dependent response at 50% of maximal expression of a particular response that is induced, provoked or potentiated by the particular test compound.

The methods described herein include administering to a subject in need a composition containing a therapeutically effective amount of one or more reversible or irreversible Btk inhibitor compounds described herein. Without being bound by a particular theory, the diverse roles played by Btk signaling in various hematopoietic cell functions, e.g., B-cell receptor activation, suggests that small molecule Btk inhibitors are useful for reducing the risk of or treating a variety of diseases affected by or affecting many cell types of the hematopoetic lineage including, e.g., autoimmune diseases, heteroimmune conditions or diseases, inflammatory diseases, cancer (e.g., B-cell proliferative disorders), and thromboembolic disorders. Further, the irreversible Btk inhibitor compounds described herein can be used to inhibit a small subset of other tyrosine kinases that share homology with Btk by having a cysteine residue (including a Cys 481 residue) that can form a covalent bond with the irreversible inhibitor. Thus, a subset of tyrosine kinases other than Btk are also expected to be useful as therapeutic targets in a number of health conditions.

In some embodiments, the compositions and methods described herein can be used to treat an autoimmune disease, which includes, but is not limited to, rheumatoid arthritis, psoriatic arthritis, osteoarthritis, Still's disease, juvenile arthritis, lupus, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, autoimmune hemolytic anemia, warm autoimmune hemolytic anemia, cold hemolytic anemia, Wegener's granulomatosis, psoriasis, alopecia universalis, Behçet's disease, chronic fatigue, dysautonomia, endometriosis, interstitial cystitis, neuromyotonia, scleroderma, immune-mediated thrombocytopenia, and vulvodynia.

In some embodiments, the compositions and methods described herein can be used to treat heteroimmune conditions or diseases, which include, but are not limited to graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

In some embodiments, the compositions and methods described herein can be used to treat ischemia/reperfusion injury, such as ischemia/reperfusion injury caused by transplantation, heart attack, stroke, or the like.

In some embodiments, the compositions and methods described herein can be used to treat an inflammatory disease, which includes, but is not limited to asthma, inflammatory bowel disease, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, and vulvitis.

In some embodiments, the compositions and methods described herein can be used to treat a cancer, e.g., B-cell proliferative disorders, which include, but are not limited to diffuse large B cell lymphoma, follicular lymphoma, chronic lymphocytic lymphoma, chronic lymphocytic leukemia, B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, and lymphomatoid granulomatosis.

In some embodiments, the compositions and methods described herein can be used to treat thromboembolic disorders, which include, but are not limited to myocardial infarct, angina pectoris (including unstable angina), reocclusions or restenoses after angioplasty or aortocoronary bypass, stroke, transitory ischemia, peripheral arterial occlusive disorders, pulmonary embolisms, and deep venous thromboses.

In some embodiments, the compositions and methods described herein can be used to treat a solid tumor. In some embodiments, the composition is for use in treatment of a sarcoma or carcinoma. In some embodiments, the composition is for use in treatment of a sarcoma. In some embodiments, the composition is for use in treatment of a carcinoma. In some embodiments, the sarcoma is selected from alveolar rhabdomyosarcoma; alveolar soft part sarcoma; ameloblastoma; angiosarcoma; chondrosarcoma; chordoma; clear cell sarcoma of soft tissue; dedifferentiated liposarcoma; desmoid; desmoplastic small round cell tumor; embryonal rhabdomyosarcoma; epithelioid fibrosarcoma; epithelioid hemangioendothelioma; epithelioid sarcoma; esthesioneuroblastoma; Ewing sarcoma; extrarenal rhabdoid tumor; extraskeletal myxoid chondrosarcoma; extrasketetal osteosarcoma; fibrosarcoma; giant cell tumor; hemangiopericytoma; infantile fibrosarcoma; inflammatory myofibroblastic tumor; Kaposi sarcoma; leiomyosarcoma of bone; liposarcoma; liposarcoma of bone; malignant fibrous histiocytoma (MFH); malignant fibrous histiocytoma (MFH) of bone; malignant mesenchymoma; malignant peripheral nerve sheath tumor; mesenchymal chondrosarcoma; myxofibrosarcoma; myxoid liposarcoma; myxoinflammatory fibroblastic sarcoma; neoplasms with perivascular epitheioid cell differentiation; osteosarcoma; parosteal osteosarcoma; neoplasm with perivascular epitheioid cell differentiation; periosteal osteosarcoma; pleomorphic liposarcoma; pleomorphic rhabdomyosarcoma; PNET/extraskeletal Ewing tumor; rhabdomyosarcoma; round cell liposarcoma; small cell osteosarcoma; solitary fibrous tumor; synovial sarcoma; telangiectatic osteosarcoma. In some embodiments, the carcinoma is selected from an adenocarcinoma, squamous cell carcinoma, adenosquamous carcinoma, anaplastic carcinoma, large cell carcinoma, or small cell carcinoma. In some embodiments, the solid tumor is selected from anal cancer; appendix cancer; bile duct cancer (i.e., cholangiocarcinoma); bladder cancer; brain tumor; breast cancer; HER2-amplified breast cancer; cervical cancer; colon cancer; cancer of Unknown Primary (CUP); esophageal cancer; eye cancer; fallopian tube cancer; kidney cancer; renal cell carcinoma; liver cancer; lung cancer; medulloblastoma; melanoma; oral cancer; ovarian cancer; pancreatic cancer; pancreatic ductal cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer. In some embodiments, the carcinoma is breast cancer. In some embodiments, the breast cancer is invasive ductal carcinoma, ductal carcinoma in situ, invasive lobular carcinoma, or lobular carcinoma in situ. In some embodiments, the carcinoma is pancreatic cancer. In some embodiments, the pancreatic cancer is adenocarcinoma, or islet cell carcinoma. In some embodiments, the carcinoma is colorectal cancer. In some embodiments, the colorectal cancer is adenocarcinoma. In some embodiments, the solid tumor is a colon polyp. In some embodiments, the colon polyp is associated with familial adenomatous polyposis. In some embodiments, the carcinoma is bladder cancer. In some embodiments, the bladder cancer is transitional cell bladder cancer, squamous cell bladder cancer, or adenocarcinoma. In some embodiments, the carcinoma is lung cancer. In some embodiments, the lung cancer is a non-small cell lung cancer. In some embodiments, the non-small cell lung cancer is adenocarcinoma, squamous-cell lung carcinoma, or large-cell lung carcinoma. In some embodiments, the non-small cell lung cancer is large cell lung cancer. In some embodiments, the lung cancer is a small cell lung cancer. In some embodiments, the carcinoma is prostate cancer. In some embodiments, the prostate cancer is adenocarcinoma or small cell carcinoma. In some embodiments, the carcinoma is ovarian cancer. In some embodiments, the ovarian cancer is epithelial ovarian cancer. In some embodiments, the carcinoma is bile duct cancer. In some embodiments, the bile duct cancer is proximal bile duct carcinoma or distal bile duct carcinoma.

In some embodiments, the compositions and methods described herein can be used to treat mastocytosis.

In some embodiments, the compositions and methods described herein can be used to treat carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, or Smoldering of indolent multiple myeloma.

In some embodiments, the compositions and methods described herein can be used to treat a central nervous system (CNS) malignancy. In some embodiments, the CNS malignancy is a primary CNS lymphoma. In some embodiments the primary CNS lymphoma is a glioma. In some embodiments the glioma is astrocytomas, ependymomas, oligodendrogliomas. In some embodiments the CNS malignancy is astrocytic tumors such as juvenile pilocytic, subependymal, well differentiated or moderately differentiated anaplastic astrocytoma; anaplastic astrocytoma; glioblastoma multiforme; ependymal tumors such as myxopapillary and well-differentiated ependymoma, anaplastic ependymoma, ependymoblastoma; oligodendroglial tumors including well-differentiated oligodendroglioma and anaplastic oligodendroglioma; mixed tumors such as mixed astrocytoma-ependymoma, mixed astrocytoma-oligodendroglioma, mixed astrocytomaependymoma-oligodendroglioma; or medulloblastoma.

In some embodiments, the compositions and methods described herein can be used to treat hematological malignancies such as, but not limited to, a leukemia, a lymphoma, a myeloma, a non-Hodgkin's lymphoma, a Hodgkin's lymphoma, a T-cell malignancy, or a B-cell malignancy. In some embodiments, the hematological malignancy is a treatment naïve hematological malignancy. In some embodiments the hematological malignancy is a relapsed or refractory hematological malignancy.

In some embodiments, the hematologic malignancy is a T-cell malignancy. In some embodiments, the T-cell malignancy is peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas. In some embodiments, the T-cell malignancy is a relapsed or refractory T-cell malignancy. In some embodiments, the T-cell malignancy is a treatment naïve T-cell malignancy. In some embodiments, the hematologic malignancy is a B-cell proliferative disorder.

In some embodiments, the cancer is chronic lymphocytic leukemia (CLL), small lymphocytic lymphoma (SLL), high risk CLL, a non-CLL/SLL lymphoma, or prolymphocytic leukemia (PLL). In some embodiments, the cancer is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, DLBCL is further divided into subtypes: activated B-cell diffuse large B-cell lymphoma (ABC-DLBCL), germinal center diffuse large B-cell lymphoma (GCB DLBCL), and Double-Hit (DH) DLBCL. In some embodiments, ABC-DLBCL is characterized by a CD79B mutation. In some embodiments, ABC-DLBCL is characterized by a CD79A mutation. In some embodiments, the ABC-DLBCL is characterized by a mutation in MyD88, A20, or a combination thereof. In some embodiments, the cancer is acute or chronic myelogenous (or myeloid) leukemia, myelodysplastic syndrome, or acute lymphoblastic leukemia. In some embodiments, the B-cell proliferative disorder is a relapsed and refractory B-cell proliferative disorder. In some embodiments, the B-cell proliferative disorder is a treatment naïve B-cell proliferative disorder.

In some embodiments, the compositions and methods described herein can be used to treat a hematological malignancy (including leukemia, peripheral T-cell lymphoma, anaplastic large cell lymphoma, angioimmunoblastic lymphoma, cutaneous T-cell lymphoma, adult T-cell leukemia/lymphoma, blastic NK-cell lymphoma, lymphoblastic lymphoma, NK/T-cell lymphoma, treatment-related T cell lymphoma, T-cell acute lymphoblastic leukemia (T-cell ALL), T-cell polymorphocytic leukemia, or large granular lymphocytic leukemia diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulinemia (WM), splenic marginal zone lymphoma, multiple myeloma, plasmacytoma, intravascular large B-cell lymphoma). In an embodiment the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis.

In some embodiments, the compositions and methods described herein can be used to treat fibrosis. In some embodiments, the fibrosis is not associated with graft versus host disease (GVHD). In some embodiments, the fibrosis is not associated with sclerodermatous GVHD, lung chronic GVHD, or liver chronic GVHD. In some embodiments, the fibrosis is of the liver, lung, pancreas, kidney, bone marrow, heart, skin, intestine, or joints. In some embodiments, the fibrosis is of the liver. In some embodiments, the fibrosis is of the lung. In some embodiments, the fibrosis is of the pancreas. In some embodiments, the patient has cirrhosis, chronic pancreatitis, or cystic fibrosis.

In further embodiments, the compositions and methods described herein can be used to treat thromboembolic disorders, which include, but are not limited to myocardial infarct, angina pectoris (including unstable angina), reocclusions or restenoses after angioplasty or aortocoronary bypass, stroke, transitory ischemia, peripheral arterial occlusive disorders, pulmonary embolisms, and deep venous thromboses.

Symptoms, diagnostic tests, and prognostic tests for each of the above-mentioned conditions are known in the art. See, e.g., Harrison's Principles of Internal Medicine©,” 16th ed., 2004, The McGraw-Hill Companies, Inc. Dey et al. (2006), Cytojournal 3(24), and the “Revised European American Lymphoma” (REAL) classification system (see, e.g., the website maintained by the National Cancer Institute).

A number of animal models are useful for establishing a range of therapeutically effective doses of reversible or irreversible Btk inhibitor compounds for treating any of the foregoing diseases.

For example, dosing of reversible or irreversible Btk inhibitor compounds for treating an autoimmune disease can be assessed in a mouse model of rheumatoid arthritis. In this model, arthritis is induced in Balb/c mice by administering anti-collagen antibodies and lipopolysaccharide. See Nandakumar et al. (2003), Am. J. Pathol 163:1827-1837.

In another example, dosing of reversible or irreversible Btk inhibitors for the treatment of B-cell proliferative disorders can be examined in, e.g., a human-to-mouse xenograft model in which human B-cell lymphoma cells (e.g. Ramos cells) are implanted into immunodeficient mice (e.g., “nude” mice) as described in, e.g., Pagel et al. (2005), Clin Cancer Res 11(13):4857-4866.

Animal models for treatment of thromboembolic disorders are also known.

The therapeutic efficacy of the compound for one of the foregoing diseases can be optimized during a course of treatment. For example, a subject being treated can undergo a diagnostic evaluation to correlate the relief of disease symptoms or pathologies to inhibition of in vivo Btk activity achieved by administering a given dose of an irreversible Btk inhibitor. Cellular assays known in the art can be used to determine in vivo activity of Btk in the presence or absence of an irreversible Btk inhibitor. For example, since activated Btk is phosphorylated at tyrosine 223 (Y223) and tyrosine 551 (Y551), phospho-specific immunocytochemical staining of P-Y223 or P-Y551-positive cells can be used to detect or quantify activation of Btk in a population of cells (e.g., by FACS analysis of stained vs unstained cells). See, e.g., Nisitani et al. (1999), Proc. Natl. Acad. Sci, USA 96:2221-2226. Thus, the amount of the Btk inhibitor compound that is administered to a subject can be increased or decreased as needed so as to maintain a level of Btk inhibition optimal for treating the subject's disease state.

### Compounds

In the following description of reversible or irreversible Btk compounds suitable for use in the methods described herein, definitions of referred-to standard chemistry terms may be found in reference works (if not otherwise defined herein), including Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the ordinary skill of the art are employed. In addition, nucleic acid and amino acid sequences for Btk (e.g., human Btk) are known in the art as disclosed in, e.g., U.S. Pat. No. 6,326,469. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients.

In certain embodiments, the Btk inhibitor compounds of the invention are selective for Btk and kinases having a cysteine residue in an amino acid sequence position of the tyrosine kinase that is homologous to the amino acid sequence position of cysteine 481 in Btk.

Generally, a reversible or irreversible inhibitor compound of Btk used in the methods described herein can be identified or characterized in an in vitro assay, e.g., an acellular biochemical assay or a cellular functional assay. Such assays are useful to determine an in vitro IC₅₀ for a reversible or irreversible Btk inhibitor compound.

For example, an acellular kinase assay can be used to determine Btk activity after incubation of the kinase in the absence or presence of a range of concentrations of a candidate irreversible Btk inhibitor compound. If the candidate compound is in fact an irreversible Btk inhibitor, Btk kinase activity will not be recovered by repeat washing with inhibitor-free medium. See, e.g., J. B. Smaill, et al. (1999), J. Med. Chem. 42(10):1803-1815. Further, covalent complex formation between Btk and a candidate irreversible Btk inhibitor is a useful indicator of irreversible inhibition of Btk that can be readily determined by a number of methods known in the art (e.g., mass spectrometry). For example, some irreversible Btk-inhibitor compounds can form a covalent bond with Cys 481 of Btk (e.g., via a Michael reaction).

Cellular functional assays for Btk inhibition include measuring one or more cellular endpoints in response to stimulating a Btk-mediated pathway in a cell line (e.g., BCR activation in Ramos cells) in the absence or presence of a range of concentrations of a candidate irreversible Btk inhibitor compound. Useful endpoints for determining a response to BCR activation include, e.g., autophosphorylation of Btk, phosphorylation of a Btk target protein (e.g., PLC-γ), and cytoplasmic calcium flux.

High throughput assays for many acellular biochemical assays (e.g., kinase assays) and cellular functional assays (e.g., calcium flux) are well known to those of ordinary skill in the art. In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. Automated systems thereby allow the identification and characterization of a large number of reversible or irreversible Btk compounds without undue effort.

Reversible or irreversible Btk inhibitor compounds can be used for the manufacture of a medicament for treating any of the foregoing conditions (e.g., autoimmune diseases, inflammatory diseases, allergy disorders, B-cell proliferative disorders, or thromboembolic disorders).

In some embodiments, the reversible or irreversible Btk inhibitor compound used for the methods described herein inhibits Btk or a Btk homolog kinase activity with an in vitro IC₅₀ of less than about 10 μM, less than about 1 μM, less than about 0.5 μM, less than about 0.4 μM, less than about 0.3 μM, less than about 0.1 μM, less than about 0.08 μM, less than about 0.06 μM, less than about 0.05 μM, less than about 0.04 μM, less than about 0.03 μM, less than about 0.02 μM, less than about 0.01 μM, less than about 0.008 μM, less than about 0.006 μM, less than about 0.005 μM, less than about 0.004 μM, less than about 0.003 μM, less than about 0.002 μM, less than about 0.001 μM, less than about 0.00099 μM, less than about 0.00098 μM, less than about 0.00097 μM, less than about 0.00096 μM, less than about 0.00095 μM, less than about 0.00094 μM, less than about 0.00093 μM, less than about 0.00092, or less than about 0.00090 μM.

In one embodiment, the Btk inhibitor compound selectively inhibits an activated form of its target tyrosine kinase (e.g., a phosphorylated form of the tyrosine kinase). For example, activated Btk is transphosphorylated at tyrosine 551. Thus, in these embodiments the Btk inhibitor inhibits the target kinase in cells only once the target kinase is activated by a signaling event.

Described herein are compounds of any of Formula (I), (II), (III), (IIIa) (IVa)-(IVd), (Va)-(Vd), (VIa)-(VId) or (VIIa)-(VIId). Also described herein are pharmaceutically acceptable salts and stereoisomers of such compounds. Pharmaceutical compositions that include at least one such compound or a pharmaceutically acceptable salt or stereoisomer of such compound, are provided. In some embodiments, when compounds disclosed herein contain an oxidizable nitrogen atom, the nitrogen atom can be converted to an N-oxide by methods well known in the art. In certain embodiments, isomers and chemically protected forms of compounds having a structure represented by any of Formula (I), (II), (III), (IIIa), (IVa)-(IVd), (Va)-(Vd), (VIa)-(VId) or (VIIa)-(VIId) are also provided. In one aspect, provided herein is a compound of Formula (I) having the structure:

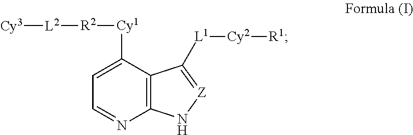

or a pharmaceutically acceptable salt thereof; or a stereoisomer or an isotopic variant thereof;
wherein:
Z is C(R⁹), or N; R⁹ is H, halo, substituted or unsubstituted C₁-C₆alkyl, OR^{13a}, —NR^{13a}R^{13b}, —SR^{13a}, C₁-C₄alkoxyC₁-C₄alkyl, hydroxyC₁-C₄alkyl, haloC₁-C₄alkyl, haloC₁-C₄alkoxy, cyano, substituted or unsubstituted C₃-C₅cycloalkyl, substituted or unsubstituted C₂-C₇heterocycloalkyl, substituted or unsubstituted aryl, or substituted or unsubstituted heteroaryl; each R^{13a} and R^{13b} is independently H, substituted or unsubstituted C₁-C₆alkyl, or substituted or unsubstituted C₃-C₈cycloalkyl;
Cy¹ is substituted or unsubstituted aryl or substituted or unsubstituted heteroaryl;
Cy² is substituted or unsubstituted C₃-C₈cycloalkyl, substituted or unsubstituted C₂-C₇heterocycloalkyl, substituted or unsubstituted C₂-C₇heterocycloalkenyl, or substituted or unsubstituted C₃-C₈cycloalkenyl;
Cy³ is substituted or unsubstituted C₃-C₈cycloalkyl, substituted or unsubstituted C₂-C₇heterocycloalkyl, substituted or unsubstituted C₂-C₇heterocycloalkenyl, or substituted or unsubstituted C₃-C₈cycloalkenyl, substituted or unsubstituted aryl, or substituted or unsubstituted heteroaryl;
L¹ is a single bond, substituted or unsubstituted C₁-C₄alkylene, —N(R⁵)—, —O—, or —S—; R⁵ is H, substituted or unsubstituted C₁-C₆alkyl, substituted or unsubstituted C₃-C₈cycloalkyl, or —C(O)—R^{5a}; R^{5a} is substituted or unsubstituted C₁-C₄alkyl, substituted or unsubstituted C₃-C₈cycloalkyl, substituted or unsubstituted C₂-C₇heterocycloalkyl, substituted or unsubstituted aryl, substituted or unsubstituted heteroaryl;
L² is —N(R^{10a})C(O)—, —C(O)N(R^{10a})—, —N(R^{10a})C(O)N(R^{10b})—, —O—, —S—, —S(O)—, —S(O)ₚ—, —N(R^{10a})S(O)ₚ—, or —S(O)ₚN(R^{10a})—; or L² and Cy³, taken together with the atoms to which they are attached, form a 9-14 membered bicyclic or tricyclic heterocyclyl which is unsubstituted or substituted with one or more substituents selected from C₁-C₆ alkyl, C₃-C₆ cycloalkyl, hydroxy, and carbonyl; each R^{10a} and R^{10b} is independently H, substituted or unsubstituted C₁-C₆alkyl, or substituted or unsubstituted C₃-C₈cycloalkyl; p is 1 or 2;
R¹ is H, halo, substituted or unsubstituted C₁-C₆alkyl, —OR^{12a}, —NR^{12a}R^{12b}, —SR^{12a}, —C(O)—O—R^{12a}, —C(O)—C(O)—N(R^{12a})R^{12b}, —C(O)—N(R^{12a})R^{12b}, —S(O)ₚ—N(R^{12a})R^{12b}, —C₁-C₄alkoxyC₁-C₄alkyl, hydroxyC₁-C₄alkyl, haloC₁-C₄alkyl, haloC₁-C₄alkoxy, cyano, substituted or unsubstituted C₃-C₈cycloalkyl, substituted or unsubstituted C₂-C₇heterocycloalkyl, substituted or unsubstituted aryl, or substituted or unsubstituted heteroaryl; each R^{12a} and R^{12b} is independently H, substituted or unsubstituted C₁-C₆alkyl, or substituted or unsubstituted C₃-C₈cycloalkyl; or R¹ is —C(O)—R^{1a}, —C(S)—R^{1a}, —S(O)_{q}—R^{1a}; —N(R^{12a})—C(O)R^{1a}, or —N(R^{12a})—S(O)_{q}R^{1a}; R^{1a} is substituted or unsubstituted C₁-C₄alkyl, substituted or unsubstituted C₂-C₄alkenyl, substituted or unsubstituted C₂-C₄alkynyl, substituted or unsubstituted C₃-C₈cycloalkyl, substituted or unsubstituted C₂-C₇heterocycloalkyl, substituted or unsubstituted aryl, or substituted or unsubstituted heteroaryl; q is 1 or 2; and
R² is a single bond, substituted or unsubstituted C₁-C₄alkylene, or substituted or unsubstituted C₃-C₆cycloalkylene.

In one embodiment, —R²-L²-Cy³ is —R²—N(R^{10a})C(O)-Cy³, —R²—C(O)N(R^{10a})-Cy³, or —R²—O-Cy³. In another embodiment, —R²-L²-Cy³ is —R²—N(R^{10a})C(O)-Cy³, or —R²—C(O)N(R^{10a})-Cy³. In a particular embodiment, —R²-L²-Cy³ is —R²—N(H)C(O)-Cy³.

In one embodiment, R^{10a} is H. In another embodiment, R^{10a} is substituted or unsubstituted C₁-C₆alkyl, or substituted or unsubstituted C₃-C₈cycloalkyl. In another embodiment, R^{10a} is unsubstituted C₁-C₆alkyl. In another embodiment, R^{10a} is C₁-C₆alkyl, substituted with hydroxyl. In one particular embodiment, R^{10a} is H, Me, Et, i-Pr, or n-Pr.

In one embodiment, R² is unsubstituted C₁-C₄alkylene or C₁-C₄alkylene substituted with —OH, halo, or C₁-C₄alkyl. In another embodiment, R² is —CH₂—, —C(H)Me-, —C(Me)₂-, or cyclopropyl.

In another aspect, provided herein is a compound of Formula (II) having the structure:

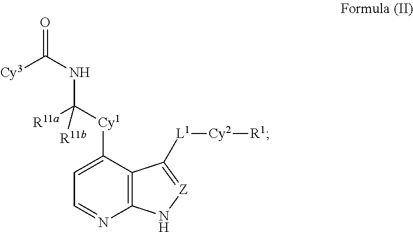

or a pharmaceutically acceptable salt thereof; or a stereoisomer or an isotopic variant thereof; wherein Cy¹, Cy², Cy³, L¹, R¹ and Z are as described for Formula (I); each R^{11a} and R^{11b} is independently H or substituted or unsubstituted C₁-C₄alkyl; or R^{11a} and R^{11b} may join together with the carbon atom to which they are attached to form a substituted or unsubstituted C₃-C₆cycloalkylene; and wherein the substitutions on R^{11a} and R^{11b}, if present, are independently selected from —OH, halo, or C₁-C₄alkyl.

In some embodiments, Cy¹ is substituted or unsubstituted phenyl, substituted or unsubstituted pyridyl, or substituted or unsubstituted pyrimidinyl. In one embodiment, Cy¹ is substituted or unsubstituted aryl. In another embodiment, Cy¹ is unsubstituted aryl. In another embodiment, Cy¹ is aryl substituted with one or more groups selected from halo, CN, C₁-C₄alkyl, haloC₁-C₄alkyl, C₃-C₈cycloalkyl, hydroxyl, and C₁-C₄alkoxy. In another embodiment, Cy¹ is aryl substituted with one or more of Cl, F, CN, Me, Et, t-Bu, CHF₂, CF₃, cyclopropyl, hydroxyl, or methoxy.

In another embodiment, Cy¹ is substituted or unsubstituted phenyl. In another embodiment, Cy¹ is unsubstituted phenyl. In another embodiment, Cy¹ is phenyl substituted with one or more groups selected from halo, CN, C₁-C₄alkyl, haloC₁-C₄alkyl, C₃-C₈cycloalkyl, hydroxyl, and C₁-C₄alkoxy. In another embodiment, Cy¹ is phenyl substituted with one or more groups selected from Cl, F, CN, Me, Et, t-Bu, CHF₂, CF₃, cyclopropyl, hydroxyl, and methoxy. In some embodiments, Cy¹ is substituted or unsubstituted phenyl.

In one embodiment, Cy¹ is substituted or unsubstituted heteroaryl. In another embodiment, Cy¹ is unsubstituted heteroaryl. In another embodiment, Cy¹ is heteroaryl substituted with one or more groups selected from halo, CN, C₁-C₄alkyl, haloC₁-C₄alkyl, C₃-C₈cycloalkyl, hydroxyl, and C₁-C₄alkoxy. In another embodiment, Cy¹ is heteroaryl substituted with one or more groups selected from Cl, F, CN, Me, Et, t-Bu, CHF₂, CF₃, cyclopropyl, hydroxyl, and methoxy. In one embodiment, the heteroaryl is pyridyl or pyrimidinyl.

In one embodiment, Cy¹ is substituted or unsubstituted pyridyl. In another embodiment, Cy¹ is unsubstituted pyridyl. In another embodiment, Cy¹ is pyridyl substituted with one or more groups selected from halo, CN, C₁-C₄alkyl, haloC₁-C₄alkyl, C₃-C₈cycloalkyl, hydroxyl, and C₁-C₄alkoxy. In another embodiment, Cy¹ is pyridyl substituted with one or more groups selected from Cl, F, CN, Me, Et, t-Bu, CHF₂, CF₃, cyclopropyl, hydroxyl, and methoxy.

In one aspect, provided herein is a compound of Formula (III) having the structure:

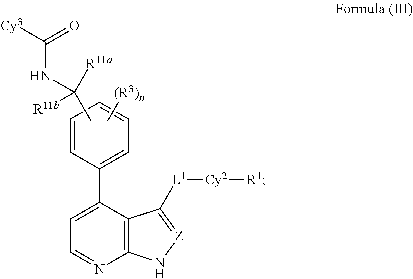

or a pharmaceutically acceptable salt thereof; or a stereoisomer or an isotopic variant thereof; wherein Cy², Cy³, L¹, R¹ and Z are as described for Formula (I); and each R^{11a} and R^{11b} is independently H or substituted or unsubstituted C₁-C₄alkyl; or R^{11a} and R^{11b} may join together with the carbon atom to which they are attached to form a substituted or unsubstituted C₃-C₆cycloalkylene; and wherein the substitutions on R^{11a} and R^{11b}, if present, are independently selected from —OH, halo, or C₁-C₄alkyl; and
each R³ is each independently halo, substituted or unsubstituted C₁-C₆alkyl, —OR²¹a, —NR^{21a}R^{21b}, —SR^{21a}, —C(O)—O—R^{21a}, —C(O)—C(O)—N(R^{21a})R^{21b}, —C(O)—N(R^{21a})R^{21b}, —N(R^{21a})C(O)—R^{21b}, or —S(O)ₜ—N(R^{21a})R^{21b}; C₁-C₄alkoxyC₁-C₄alkyl, hydroxyC₁-C₄alkyl, haloC₁-C₄alkyl, haloC₁-C₄alkoxy, cyano, substituted or unsubstituted C₃-C₈cycloalkyl, substituted or unsubstituted C₂-C₇heterocycloalkyl, substituted or unsubstituted aryl, substituted or unsubstituted heteroaryl; each R^{21a} and R^{21b} is independently H, substituted or unsubstituted C₁-C₆alkyl, or substituted or unsubstituted C₃-C₈cycloalkyl; and n is 0-4.

In one embodiment, the group —C(R^{11a})(R^{11b})—N(H)—C(O)-Cy³ is at 2-position. In another embodiment, it is at 3- or 4-position.

In one embodiment, L¹ is a single bond, —N(R⁵)—, or —O—. In another embodiment, L¹ is a single bond. In another embodiment, L¹ is-N(R⁵)— or —O—. In another embodiment, L¹ is-N(R⁵)—. In one embodiment, R⁵ is H or Me. In one particular embodiment, R⁵ is H.

In one aspect, provided herein is a compound of Formula (IVa) or (IVb) having the structure:

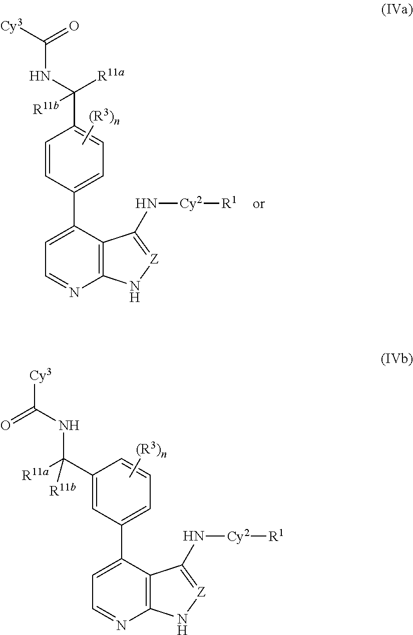

or a pharmaceutically acceptable salt thereof; or a stereoisomer or an isotopic variant thereof; wherein Cy², Cy³, R¹ and Z are as described for Formula (I); and each R^{11a} and R^{11b} is independently H or substituted or unsubstituted C₁-C₄alkyl; or R^{11a} and R^{11b} may join together with the carbon atom to which they are attached to form a substituted or unsubstituted C₃-C₆cycloalkylene; and wherein the substitutions on R^{11a} and Rub, if present, are independently selected from —OH, halo, or C₁-C₄alkyl; and
each R³ is each independently halo, substituted or unsubstituted C₁-C₆alkyl, —OR²¹a, —NR^{21a}R^{21b}, —SR^{21a}, —C(O)—O—R^{21a}, —C(O)—C(O)—N(R^{21a})R^{21b}, —C(O)—N(R^{21a})R^{21b}, —N(R^{21a})C(O)—R^{21b}, or —S(O)ₜ—N(R^{21a})R^{21b}; C₁-C₄alkoxyC₁-C₄alkyl, hydroxyC₁-C₄alkyl, haloC₁-C₄alkyl, haloC₁-C₄alkoxy, cyano, substituted or unsubstituted C₃-C₈cycloalkyl, substituted or unsubstituted C₂-C₇heterocycloalkyl, substituted or unsubstituted aryl, substituted or unsubstituted heteroaryl;
each R^{21a} and R^{21b} is independently H, substituted or unsubstituted C₁-C₆alkyl, or substituted or unsubstituted C₃-C₈cycloalkyl; and n is 0-4.

In one embodiment, Cy² is substituted or unsubstituted C₃-C₈cycloalkyl, or substituted or unsubstituted C₃-C₈cycloalkenyl. In another embodiment, Cy² is substituted or unsubstituted C₃-C₇cycloalkyl. In another embodiment, Cy² is cyclopropyl, cyclobutyl, cyclopentyl, cyclohexyl, or cycloheptyl.

In one embodiment, Cy² is substituted or unsubstituted C₂-C₇heterocycloalkyl, or substituted or unsubstituted C₂-C₇heterocycloalkenyl. In another embodiment, Cy² is substituted or unsubstituted C₂-C₇heterocycloalkyl. In another embodiment, Cy² is substituted or unsubstituted pyrrolidinyl, substituted or unsubstituted piperidinyl, substituted or unsubstituted morpholinyl, or substituted or unsubstituted piperizinyl, oxanyl, 1,1-dioxo-1λ⁶-thiomorpholinyl, 2-oxo-pyrrolidinyl, pyrrolidin-3-ylidene, 2,3-dioxopiperazinyl, or 1,1-dioxo-1λ⁶-thianyl. In another embodiment, Cy² is substituted or unsubstituted pyrrolidinyl, substituted or unsubstituted piperidinyl, substituted or unsubstituted morpholinyl, or substituted or unsubstituted piperazinyl. In another embodiment, Cy² is substituted or unsubstituted pyrrolidinyl, substituted or unsubstituted piperidinyl, or substituted or unsubstituted morpholinyl. In another embodiment, Cy² is substituted or unsubstituted pyrrolidinyl or substituted or unsubstituted piperidinyl. In another embodiment, Cy² is substituted or unsubstituted dihydropyrrolyl, or tetrahydropyridyl. In one embodiment, when Cy² is substituted, the substitutent is selected from one or more halo, C₁-C₄alkyl, or hydroxyC₁-C₄alkyl.

In some embodiments, Cy² is unsubstituted and R¹ is H.

In one embodiment, Z is ═C(R⁹)—. In one embodiment, R⁹ is H, halo, C₁-C₆alkyl, hydroxyC₁-C₆alkyl, haloC₁-C₆alkyl, or C₃-C₈cycloalkyl. In another embodiment, R⁹ is H, F, Me, Et, n-Pr, i-Pr, n-Bu, i-Bu, sec-Bu, t-Bu, cyclopropyl, or CF₃. In another embodiment, R⁹ is F or CF₃. In a particular embodiment, R⁹ is H.

In one embodiment, Z is ═N—.

In one aspect, provided herein is a compound of Formula (Va), (Vb), (Vc), or (Vd), having the structure:

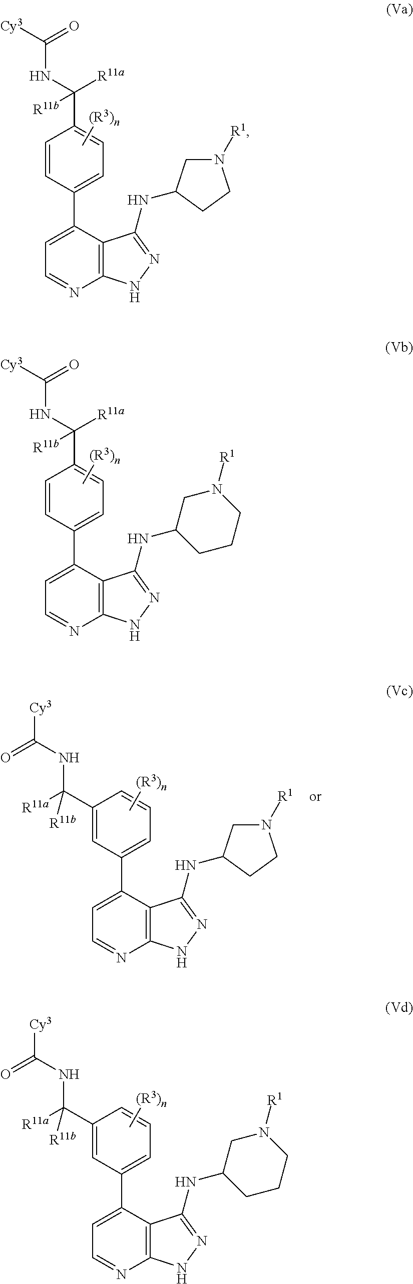

or a pharmaceutically acceptable salt thereof; or a stereoisomer or an isotopic variant thereof; wherein Cy³, R¹ and Z are as described for Formula (I); and each R^{11a} and R^{11b} is independently H or substituted or unsubstituted C₁-C₄alkyl; or R^{11a} and R^{11b} may join together with the carbon atom to which they are attached to form a substituted or unsubstituted C₃-C₆cycloalkylene; and wherein the substitutions on R^{11a} and R^{11b}, if present, are independently selected from —OH, halo, or C₁-C₄alkyl; and
each R³ is each independently halo, substituted or unsubstituted C₁-C₆alkyl, —OR^{21a}, —NR^{21a}R^{21b}, —SR^{21a}, —C(O)—O—R^{21a}, —C(O)—C(O)—N(R^{21a})R^{21b}, —C(O)—N(R^{21a})R^{21b}, —N(R^{21a})C(O)—R^{21b}, or —S(O)ₜ—N(R^{21a})R^{21b}; C₁-C₄alkoxyC₁-C₄alkyl, hydroxyC₁-C₄alkyl, haloC₁-C₄alkyl, haloC₁-C₄alkoxy, cyano, substituted or unsubstituted C₃-C₈cycloalkyl, substituted or unsubstituted C₂-C₇heterocycloalkyl, substituted or unsubstituted aryl, substituted or unsubstituted heteroaryl;
each R^{21a} and R^{21b} is independently H, substituted or unsubstituted C₁-C₆alkyl, or substituted or unsubstituted C₃-C₈cycloalkyl; and n is 0-4.

In some embodiments, R¹ is H, CN, —C(O)—R^{1a}, —C(O)—N(R^{12a})R^{12b}, N(R^{12a})R^{12b}, N(R^{12a})—C(O)—N(R^{12b})R^{12c}, —N(R^{12a})—C(O)R^{1a}, —C(S)—R^{1a}, —S(O)ₚ—R^{1a}, or —S(O)ₚ—N(R^{12a})R^{12b}. In one embodiment, R¹ is H, CN, —C(O)—R^{1a}, —C(S)—R^{1a}, —S(O)ₚ—R^{1a}, or —S(O)ₚ—N(R^{12a})R^{12b}. In some embodiments, R¹ is —C(O)—N(R^{12a})R^{12b}, N(R^{12a})R^{12b}, N(R^{12a})—C(O)—N(R^{12b})R^{12c}, or —N(R^{12a})—C(O)R^{1a}. In another embodiment, R¹ is —C(O)—R^{1a}. In some embodiments, R^{1a} is substituted or unsubstituted C₁-C₄alkyl. In one embodiment, R^{1a} is substituted or unsubstituted C₂-C₄alkenyl. In another embodiment, R^{1a} is substituted with CN, substituted or unsubstituted C₁-C₆alkyl, haloC₁-C₆alkyl, substituted or unsubstituted C₃-C₈cycloalkyl, substituted or unsubstituted aryl, substituted or unsubstituted heteroaryl, hydroxyl, substituted or unsubstituted hydroxyC₁-C₄alkyl, substituted or unsubstituted aminoC₁-C₄alkyl, or substituted or unsubstituted C₁-C₄alkoxyC₁-C₄alkyl. In another embodiment, R^{1a} is substituted or unsubstituted ethenyl. In another embodiment, R^{1a} is ethenyl and is unsubstituted or substituted with aminoC₁-C₄alkyl. In another embodiment, R^{1a} is ethenyl and is substituted with C₁-C₄alkylaminoC₁-C₄alkyl, C₃-C₈cycloalkylaminoC₁-C₄alkyl, or diC₁-C₄alkylaminoC₁-C₄alkyl. In another embodiment, R^{1a} is H, or CN.

In another embodiment, R^{1a} is a group selected from

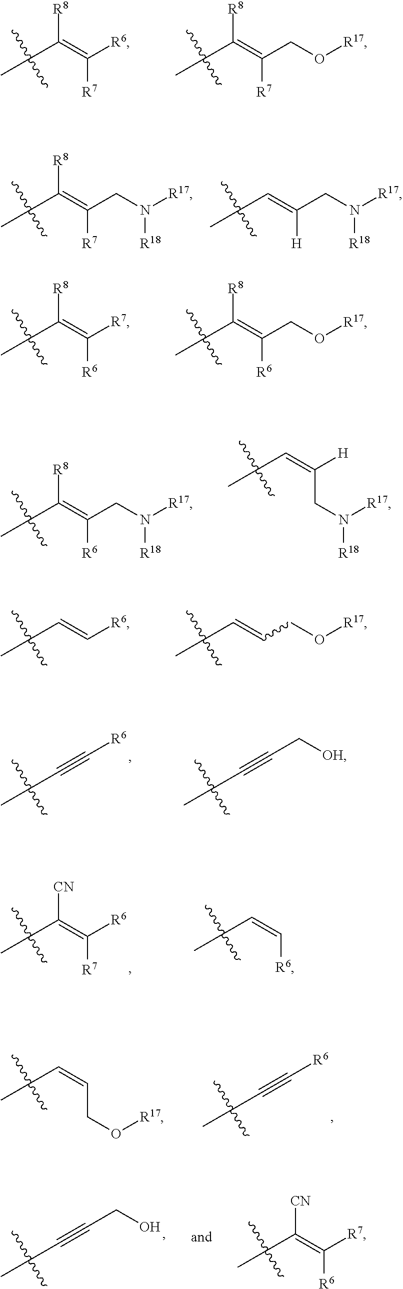

wherein
R⁶, R⁷ and R⁸ are each independently H, CN, halo, substituted or unsubstituted C₁-C₄alkyl, substituted or unsubstituted C₃-C₅cycloalkyl, substituted or unsubstituted C₂-C₇heterocycloalkyl, substituted or unsubstituted C₆-C₁₂aryl, or substituted or unsubstituted 5- to 8-membered heteroaryl; or
R⁷ and R⁸ together form a bond, thereby forming a triple bond between the carbons to which they are attached;
R¹⁷ and R¹⁸ are independently H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted 3- to 8-membered heteroaryl.

In some embodiments, when R¹⁷ or R¹⁸ is substituted or unsubstituted C₁-C₃alkyl, the substituents are selected from halo, hydroxyl, and alkoxy. In some embodiments, R¹⁷ or R¹⁸ is unsubstituted.

In some embodiments, when R¹⁷ or R¹⁸ is substituted or unsubstituted C₃-C₆cycloalkyl, substituted or unsubstituted C₂-C₇heterocycloalkyl, substituted or unsubstituted C₆-C₁₂aryl, or substituted or unsubstituted 3- to 8-membered heteroaryl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R¹⁷ or R¹⁸ is unsubstituted.

In one embodiment, R¹ is selected from the following structures:

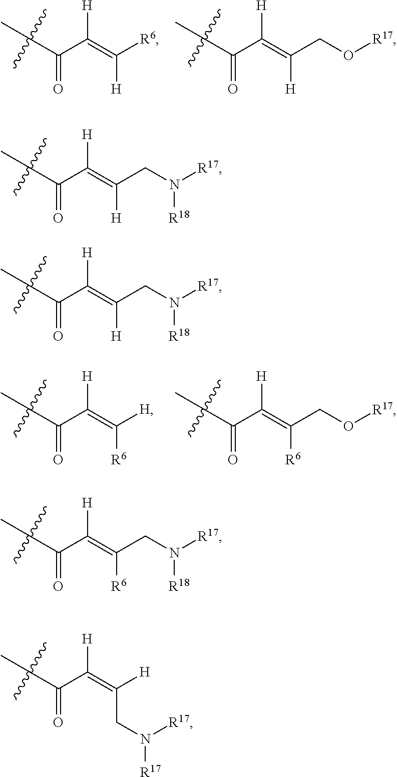

wherein:
R⁶ is CN, halo, substituted or unsubstituted C₁-C₄alkyl, substituted or unsubstituted C₃-C₈cycloalkyl, substituted or unsubstituted C₂-C₇heterocycloalkyl, substituted or unsubstituted C₆-C₁₂aryl, or substituted or unsubstituted 5- to 8-membered heteroaryl;
R⁷ and R⁸ are each H; and
R¹⁷ and R¹⁸ are independently H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted 3- to 8-membered heteroaryl.

In some embodiments, when R¹⁷ or R¹⁸ is substituted or unsubstituted C₁-C₃alkyl, the substituents are selected from halo, hydroxyl, and alkoxy. In some embodiments, R¹⁷ or R¹⁸ is unsubstituted.

In some embodiments, when R¹⁷ or R¹⁸ is substituted or unsubstituted C₃-C₆cycloalkyl, substituted or unsubstituted C₂-C₇heterocycloalkyl, substituted or unsubstituted C₆-C₁₂aryl, or substituted or unsubstituted 3- to 8-membered heteroaryl, the substituents are selected from halo, CN, C₁-C₄alkyl, haloC₁-C₄alkyl, hydroxyC₁-C₄alkyl, C₃-C₈cycloalkyl, hydroxyl, and C₁-C₄alkoxy. In some embodiments, the substituents are selected from Cl, F, CN, Me, Et, t-Bu, CHF₂, CF₃, cyclopropyl, hydroxyl, hydroxymethyl, or methoxy. In some embodiments, R¹⁷ or R¹⁸ is unsubstituted.

In this embodiment, the first row of alkenyl species has R⁶ as a non-hydrogen substituent trans to the carbonyl that connects R¹ to the parent molecular group. In the second row, the alkenyl species has R⁶ or another non-hydrogen substituent cis to the carbonyl that connects R¹ to the parent molecular group. Wherever alkenyl substituents are present on compounds of Formulas as disclosed herein, the alkenyl represents both the cis and trans stereoisomers unless otherwise indicated.

In one aspect, provided herein is a compound of Formula (VIa), (VIb), (VIc), or (VId), having the structure:

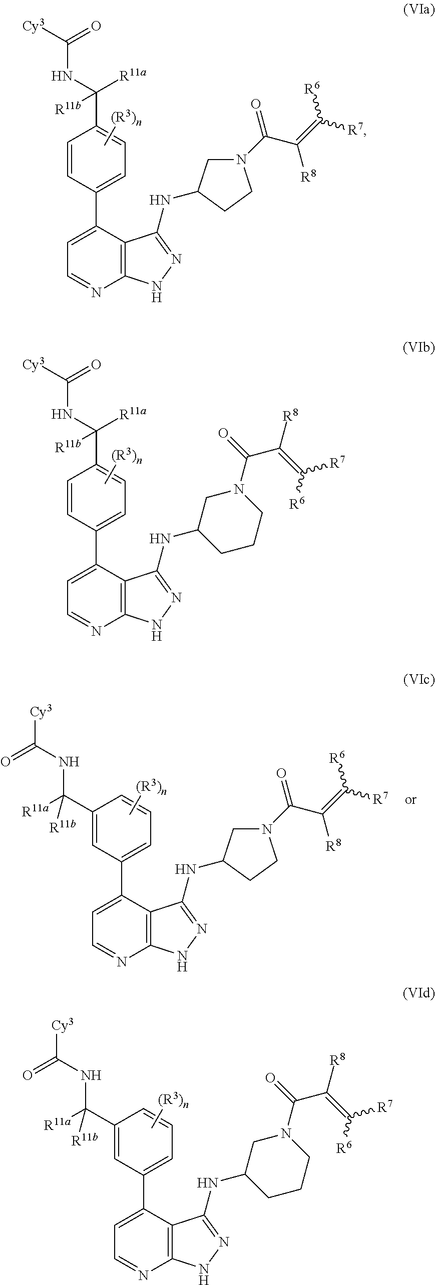

or a pharmaceutically acceptable salt thereof; or a stereoisomer or an isotopic variant thereof; wherein Cy³, and Z are as described for Formula (I); and each R^{11a} and R^{11b} is independently H or substituted or unsubstituted C₁-C₄alkyl; or R^{11a} and R^{11b} may join together with the carbon atom to which they are attached to form a substituted or unsubstituted C₃-C₆cycloalkylene; and wherein the substitutions on R^{11a} and R^{11b}, if present, are independently selected from —OH, halo, or C₁-C₄alkyl;
each R³ is each independently halo, substituted or unsubstituted C₁-C₆alkyl, —OR^{21a}, —NR^{21a}R^{21b}, —SR^{21a}, —C(O)—O—R^{21a}, —C(O)—C(O)—N(R^{21a})R^{21b}, —C(O)—N(R^{21a})R^{21b}, —N(R^{21a})C(O)—R^{21b}, or —S(O)ₜ—N(R^{21a})R^{21b}; C₁-C₄alkoxyC₁-C₄alkyl, hydroxyC₁-C₄alkyl, haloC₁-C₄alkyl, haloC₁-C₄alkoxy, cyano, substituted or unsubstituted C₃-C₈cycloalkyl, substituted or unsubstituted C₂-C₇heterocycloalkyl, substituted or unsubstituted aryl, substituted or unsubstituted heteroaryl; each R^{21a} and R^{21b} is independently H, substituted or unsubstituted C₁-C₆alkyl, or substituted or unsubstituted C₃-C₈cycloalkyl; and n is 0-4; and
R⁶, R⁷ and R⁸ are each independently H, CN, halo, substituted or unsubstituted C₁-C₄alkyl, substituted or unsubstituted C₃-C₅cycloalkyl, substituted or unsubstituted C₂-C₇heterocycloalkyl, substituted or unsubstituted C₆-C₁₂aryl, or substituted or unsubstituted 5- to 8-membered heteroaryl; or R⁷ and R⁸ together form a bond.

In one embodiment, n is 0. In another embodiment, n is 1 or 2.

In one embodiment, n is 1 or 2, and each R³ is independently halo, CN, C₁-C₄alkyl, haloC₁-C₄alkyl, C₃-C₈cycloalkyl, hydroxyl, or C₁-C₄alkoxy. In another embodiment, n is 1 or 2, and each R³ is independently Cl, F, CN, Me, Et, t-Bu, CHF₂, CF₃, cyclopropyl, hydroxyl, or methoxy.

In one aspect, provided herein is a compound of Formula (VIIa), (VIIb), (VIIc), or (VIId), having the structure:

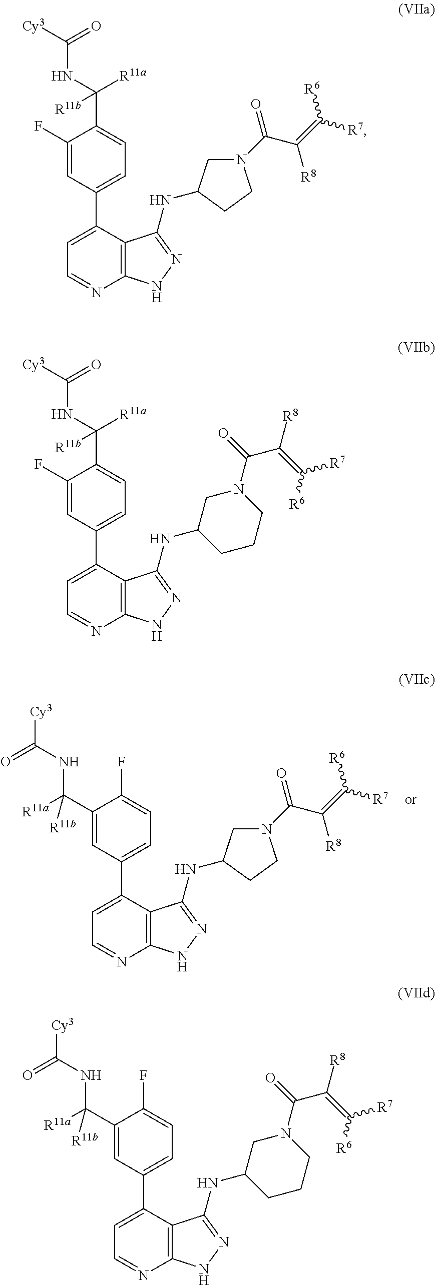

or a pharmaceutically acceptable salt thereof; or a stereoisomer or an isotopic variant thereof; wherein Cy³ is as described for Formula (I); and each R^{11a} and R^{11b} is independently H or substituted or unsubstituted C₁-C₄alkyl; or R^{11a} and R^{11b} may join together with the carbon atom to which they are attached to form a substituted or unsubstituted C₃-C₆cycloalkylene; and wherein the substitutions on R^{11a} and R^{11b}, if present, are independently selected from —OH, halo, or C₁-C₄alkyl; and
R⁶, R⁷ and R⁸ are each independently H, CN, halo, substituted or unsubstituted C₁-C₄alkyl, substituted or unsubstituted C₃-C₅cycloalkyl, substituted or unsubstituted C₂-C₇heterocycloalkyl, substituted or unsubstituted C₆-C₁₂aryl, or substituted or unsubstituted 5- to 8-membered heteroaryl; or R⁷ and R⁸ together form a bond.

In one embodiment, Cy³ is substituted or unsubstituted C₃-C₈cycloalkyl, or a substituted or unsubstituted C₂-C₇heterocycloalkyl.

In another embodiment, Cy³ is substituted or unsubstituted phenyl. In another embodiment, Cy³ is phenyl substituted with one or more of halo, CN, C₁-C₄alkyl, C₁-C₄haloalkyl, C₁-C₄hydroxyalkyl, C₃-C₈cycloalkyl, hydroxyl, or C₁-C₄alkoxy. In another embodiment, Cy³ is phenyl substituted with one or more of Me, Et, i-Pr, n-Pr, t-Bu, —C(Me)₂-OH, F, Cl, Br, —OMe, CF₃, CN, or cyclopropyl. In another embodiment, phenyl substituted with i-Pr, t-Bu, or cyclopropyl.

In another embodiment, Cy³ is substituted or unsubstituted heteroaryl. In another embodiment, Cy³ is furanyl, pyrrolyl, thienyl, imidazolyl, pyrazolyl, oxazolyl, isoxazolyl, thiazolyl, oxadiazolyl, thiadiazolyl, pyridyl, pyrimidinyl, or 4,5,6,7-tetrahydro-1,3-benzothiazole, and is substituted or unsubstituted. In another embodiment, Cy³ is furanyl, pyrrolyl, thienyl, imidazolyl, pyrazolyl, oxazolyl, isoxazolyl, thiazolyl, oxadiazolyl, thiadiazolyl, pyridyl, or pyrimidinyl, each of which is substituted with one or more of halo, CN, C₁-C₄alkyl, C₁-C₄haloalkyl, C₁-C₄hydroxyalkyl, C₃-C₈cycloalkyl, hydroxyl, or C₁-C₄alkoxy. In another embodiment, Cy³ is substituted or unsubstituted furanyl, pyrrolyl, thienyl, imidazolyl, pyrazolyl, oxazolyl, isoxazolyl, thiazolyl, oxadiazolyl, thiadiazolyl, pyridyl, or pyrimidinyl where each ring can be substituted with one or more of Me, Et, i-Pr, n-Pr, t-Bu, —C(Me)₂-OH, F, Cl, Br, —OMe, CF₃, CN, or cyclopropyl. In another embodiment, Cy³ is substituted or unsubstituted furanyl, pyrrolyl, thienyl, imidazolyl, pyrazolyl, oxazolyl, isoxazolyl, thiazolyl, oxadiazolyl, thiadiazolyl, pyridyl, or pyrimidinyl where each ring can be substituted with one or more of i-Pr, t-Bu, or cyclopropyl.

In a particular embodiment, Cy³ is unsubstituted. In another particular embodiment, Cy³ is substituted with one or more of halo, CN, C₁-C₄alkyl, haloalkyl, C₃-C₈cycloalkyl, hydroxyl, or alkoxy.

In another embodiment, Cy³ is oxazolyl, thiazolyl, oxadiazolyl, or thiadiazolyl. In another embodiment, Cy³ is oxadiazolyl. In one embodiment, Cy³ is unsubstituted or substituted with one or more of Cl, F, Me, t-Bu, cyclopropyl, or 1-hydroxy-1-methyl-ethyl.

In another embodiment, Cy³ is pyridyl. In one embodiment, Cy³ is unsubstituted or substituted with one or more of Cl, F, Me, t-Bu, or cyclopropyl

In another embodiment, Cy³ is oxazolyl, thiazolyl, oxadiazolyl, thiadiazolyl, phenyl, or pyridyl, and is substituted with one or more of Cl, F, CN, Me, Et, i-Pr, t-Bu, CHF₂, CF₃, cyclopropyl, hydroxyl, or methoxy.

In particular embodiment, Cy³ is oxadiazolyl, and is unsubstituted or substituted with i-Pr, t-Bu, or cyclopropyl

In one embodiment, R⁸ is H, F, Cl, CN, C₁-C₃alkyl, or C₃-C₆cycloalkyl.

In one embodiment, R⁸ is H, CN, Me, or cyclopropyl.

In one embodiment, each of R⁶, R⁷ and R⁸ is H.

In one embodiment, R⁷ and R⁸ are joined to form a bond, thereby forming a triple bond between the carbons to which they are attached, such that R^{1a} is ethynyl.

In one embodiment, each of R⁷ and R⁸ is H; and R⁶ is C₁-C₃alkyl or substituted C₁-C₃alkyl.

In one embodiment, R⁶ is C₁-C₃alkyl substituted with C₁-C₃alkoxy or with substituted or unsubstituted amino.

In one embodiment, R⁶ is —(CH₂)ₘ—OR^{6a} or —(CH₂)ₘ—NR^{6a}R^{6b}; m is 1, 2, 3, or 4; and each R^{6a} and R^{6b} is independently H, C₁-C₃alkyl, haloC₁-C₃alkyl, C₁-C₃alkoxy C₁-C₃alkyl, C₃-C₈cycloalkyl, C₂-C₇heterocycloalkyl, aryl, or heteroaryl.

In one embodiment, R⁶ is —(CH₂)ₘ—NR^{6a}R^{6b}.

In one embodiment, R⁶ is —(CH₂)ₘ—OR^{6a}.

In one embodiment, R^{6a} and R^{6b} are, each independently, H, cyclopropyl, Me, Et, or methoxyethyl.

In one embodiment, R⁶ is aryl or heteroaryl.

In one embodiment, R⁶ is imidazolyl, pyridyl, or pyrimidinyl

In one embodiment, R⁶ is phenyl.

In one embodiment, R⁶ is C₃-C₈cycloalkyl.

In one embodiment, R⁶ is cyclopropyl, cyclobutyl, cyclopentyl, or cyclohexyl.

In one embodiment, each R^{11a} and R^{11b} is independently H or substituted or unsubstituted C₁-C₃alkyl.

In one embodiment, each R^{11a} and R^{11b} is independently H, Me, —CH₂OH, or Et.

In one embodiment, each R^{11a} and R^{11b} is H.

In one embodiment, R^{11a} and R^{11b} may join together to form a substituted or unsubstituted C₃-C₆cycloalkyl.

In one embodiment, R^{11a} and R^{11b} may join together to form a substituted or unsubstituted cyclopropyl.

In one embodiment, Cy³ is phenyl and is unsubstituted or substituted with i-Pr, t-Bu, or cyclopropyl; R¹ is H; n is 0 or 1; R³, if present, is F, and each R^{11a} and R^{11b} is H.

In one embodiment, Cy³ is oxadiazolyl and is unsubstituted or substituted with i-Pr, t-Bu, or cyclopropyl; R¹ is H; n is 0 or 1; R³, if present, is F, and each R^{11a} and R^{11b} is H.

In some embodiments, the compound is selected from the group consisting of:
5-tert-butyl-N-{1-[4-(3-{[(3R)-pyrrolidin-3-yl]amino}-1H-pyrazolo[3,4-b]pyridin-4-yl)phenyl]cyclopropyl}-1,2,4-oxadiazole-3-carboxamide;
5-tert-butyl-N-{[2-fluoro-4-(3-{[(3R)-pyrrolidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,2,4-oxadiazole-3-carboxamide;
5-tert-butyl-N-{1-[4-(3-{[(3R)-1-(prop-2-enoyl)pyrrolidin-3-yl]amino}-1H-pyrazolo[3,4-b]pyridin-4-yl)phenyl]cyclopropyl}-1,2,4-oxadiazole-3-carboxamide;
5-tert-butyl-N-{1-[4-(3-{[(3R)-1-propanoylpyrrolidin-3-yl]amino}-1H-pyrazolo[3,4-b]pyridin-4-yl)phenyl]cyclopropyl}-1,2,4-oxadiazole-3-carboxamide;
4-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}benzamide;
N-{[4-(3-{[(3R)-1-(but-2-ynoyl)pyrrolidin-3-yl]amino}-1H-pyrazolo[3,4-b]pyridin-4-yl)-2-fluorophenyl]methyl}-4-tert-butylbenzamide;
4-tert-butyl-N-{[2-fluoro-4-(3-{[(3R)-pyrrolidin-3-yl]amino}-1H-pyrazolo[3,4-b]pyridin-4-yl)phenyl]methyl}benzamide;
4-tert-butyl-N-[2-methyl-3-(3-{[(3R)-1-(prop-2-enoyl)pyrrolidin-3-yl]amino}-1H-pyrazolo[3,4-b]pyridin-4-yl)phenyl]benzamide;
N-[3-(3-{[(3R)-1-(but-2-ynoyl)pyrrolidin-3-yl]amino}-1H-pyrazolo[3,4-b]pyridin-4-yl)-2-methylphenyl]-4-tert-butylbenzamide;
(1r,4r)-4-({4-[3-fluoro-4-(hydroxymethyl)phenyl]-1H-pyrazolo[3,4-b]pyridin-3-yl}amino)cyclohexan-1-ol;
4-tert-butyl-N-{[2-fluoro-4-(3-{[(1r,4r)-4-hydroxycyclohexyl]amino}-1H-pyrazolo[3,4-b]pyridin-4-yl)phenyl]methyl}benzamide;
4-tert-butyl-N-[3-(3-{[(1r,4r)-4-hydroxycyclohexyl]amino}-1H-pyrazolo[3,4-b]pyridin-4-yl)phenyl]benzamide;
4-tert-butyl-N-[2-methyl-3-(3-{[(1r,4r)-4-hydroxycyclohexyl]amino}-1H-pyrazolo[3,4-b]pyridin-4-yl)phenyl]benzamide;
4-tert-butyl-N-[2-fluoro-4-(3-{[(1r,4r)-4-hydroxycyclohexyl]amino}-1H-pyrazolo[3,4-b]pyridin-4-yl)phenyl]benzamide;
[2-(6-cyclopropyl-8-fluoro-1-oxo-1,2-dihydroisoquinolin-2-yl)-6-(3-{[(3R)-1-(prop-2-enoyl)pyrrolidin-3-yl]amino}-1H-pyrazolo[3,4-b]pyridin-4-yl)phenyl]methyl acetate;
6-cyclopropyl-8-fluoro-2-[2-(hydroxymethyl)-3-(3-{[(3R)-pyrrolidin-3-yl]amino}-1H-pyrazolo[3,4-b]pyridin-4-yl)phenyl]-1,2-dihydroisoquinolin-1-one;
6-cyclopropyl-8-fluoro-2-[2-(hydroxymethyl)-3-(3-{[(3R)-1-(prop-2-enoyl)pyrrolidin-3-yl]amino}-1H-pyrazolo[3,4-b]pyridin-4-yl)phenyl]-1,2-dihydroisoquinolin-1-one;
N-{[2-fluoro-4-(3-{[(1r,4r)-4-hydroxycyclohexyl]amino}-1H-pyrazolo[3,4-b]pyridin-4-yl)phenyl]methyl}-4,4-dimethylpentanamide;
1-ethyl-N-{[2-fluoro-4-(3-{[(1r,4r)-4-hydroxycyclohexyl]amino}-1H-pyrazolo[3,4-b]pyridin-4-yl)phenyl]methyl}-1H-pyrazole-4-carboxamide;
5-tert-butyl-N-{[2-fluoro-4-(3-{[(1r,4r)-4-hydroxycyclohexyl]amino}-1H-pyrazolo[3,4-b]pyridin-4-yl)phenyl]methyl}-1,2,4-oxadiazole-3-carboxamide;
4-tert-butyl-N-{1-[4-(3-{[(1r,4r)-4-hydroxycyclohexyl]amino}-1H-pyrazolo[3,4-b]pyridin-4-yl)phenyl]cyclopropyl}benzamide;
4-tert-butyl-N-{[4-(3-{[(1r,4r)-4-hydroxycyclohexyl]amino}-1H-pyrazolo[3,4-b]pyridin-4-yl)phenyl]methyl}benzamide;
N-{[4-(3-{[(3R)-1-(but-2-ynoyl)pyrrolidin-3-yl]amino}-1H-pyrazolo[3,4-b]pyridin-4-yl)-2-fluorophenyl]methyl}-5-tert-butyl-1,2,4-oxadiazole-3-carboxamide;
5-tert-butyl-N-{[4-(3-{[(3R)-1-[(2E)-4-(dimethylamino)but-2-enoyl]pyrrolidin-3-yl]amino}-1H-pyrazolo[3,4-b]pyridin-4-yl)-2-fluorophenyl]methyl}-1,2,4-oxadiazole-3-carboxamide;
N-{[2-fluoro-4-(3-{[(1r,4r)-4-hydroxycyclohexyl]amino}-1H-pyrazolo[3,4-b]pyridin-4-yl)phenyl]methyl}-4-(2-hydroxypropan-2-yl)benzamide;
5-tert-butyl-N-{[2-fluoro-4-(3-{[(1r,4r)-4-hydroxycyclohexyl]amino}-1H-pyrazolo[3,4-b]pyridin-4-yl)phenyl]methyl}-1,2-oxazole-3-carboxamide;
N-{1-[4-(3-{[(3R)-1-(but-2-ynoyl)pyrrolidin-3-yl]amino}-1H-pyrazolo[3,4-b]pyridin-4-yl)phenyl]cyclopropyl}-5-tert-butyl-1,2,4-oxadiazole-3-carboxamide;
5-tert-butyl-N-{1-[4-(3-{[(3R)-1-[(2E)-4-(dimethylamino)but-2-enoyl]pyrrolidin-3-yl]amino}-1H-pyrazolo[3,4-b]pyridin-4-yl)phenyl]cyclopropyl}-1,2,4-oxadiazole-3-carboxamide;
5-tert-butyl-N-{1-[4-(3-{[(3R)-1-(3-methyloxetane-3-carbonyl)pyrrolidin-3-yl]amino}-1H-pyrazolo[3,4-b]pyridin-4-yl)phenyl]cyclopropyl}-1,2,4-oxadiazole-3-carboxamide;
N-{[2-fluoro-4-(3-{[(3R)-pyrrolidin-3-yl]amino}-1H-pyrazolo[3,4-b]pyridin-4-yl)phenyl]methyl}benzamide;
N-{[2-fluoro-4-(3-{[(3R)-pyrrolidin-3-yl]amino}-1H-pyrazolo[3,4-b]pyridin-4-yl)phenyl]methyl}-4-methylbenzamide;
N-{[2-fluoro-4-(3-{[(3R)-1-(prop-2-enoyl)pyrrolidin-3-yl]amino}-1H-pyrazolo[3,4-b]pyridin-4-yl)phenyl]methyl}benzamide;
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-4-methylbenzamide;
4-cyclopropyl-N-{[2-fluoro-4-(3-{[(3R)-pyrrolidin-3-yl]amino}-1H-pyrazolo[3,4-b]pyridin-4-yl)phenyl]methyl}benzamide;
6-tert-butyl-2-{[2-fluoro-4-(3-{[(3R)-pyrrolidin-3-yl]amino}-1H-pyrazolo[3,4-b]pyridin-4-yl)phenyl]methyl}-1,2,3,4-tetrahydroisoquinolin-1-one;
4-tert-butyl-N-{[2-fluoro-4-(3-{[(3S)-pyrrolidin-3-yl]amino}-1H-pyrazolo[3,4-b]pyridin-4-yl)phenyl]methyl}benzamide;
6-tert-butyl-2-{[2-fluoro-4-(3-{[(1r,4r)-4-hydroxycyclohexyl]amino}-1H-pyrazolo[3,4-b]pyridin-4-yl)phenyl]methyl}-1,2,3,4-tetrahydroisoquinolin-1-one;
2-tert-butyl-N-{[2-fluoro-4-(3-{[(1r,4r)-4-hydroxycyclohexyl]amino}-1H-pyrazolo[3,4-b]pyridin-4-yl)phenyl]methyl}-1,3-oxazole-5-carboxamide;
4-cyclopropyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}benzamide;
4-cyclopropyl-N-{[2-fluoro-4-(3-{[(3R)-1-(3-methyloxetane-3-carbonyl)pyrrolidin-3-yl]amino}-1H-pyrazolo[3,4-b]pyridin-4-yl)phenyl]methyl}benzamide;
4-tert-butyl-N-[2-fluoro-4-(3-{[(3R)-pyrrolidin-3-yl]amino}-1H-pyrazolo[3,4-b]pyridin-4-yl)phenyl]benzamide;
6-tert-butyl-2-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,2,3,4-tetrahydroisoquinolin-1-one;
4-tert-butyl-N-{[2-fluoro-4-(3-{[(3S)-1-(prop-2-enoyl)pyrrolidin-3-yl]amino}-1H-pyrazolo[3,4-b]pyridin-4-yl)phenyl]methyl}benzamide;
4-tert-butyl-N-[2-fluoro-4-(3-{[(3R)-1-(prop-2-enoyl)pyrrolidin-3-yl]amino}-1H-pyrazolo[3,4-b]pyridin-4-yl)phenyl]benzamide;
3-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}benzamide;
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-4-(prop-1-en-2-yl)benzamide;
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-4-(trifluoromethyl)benzamide;
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-4-methoxybenzamide;
5-tert-butyl-N-{[2-fluoro-4-(3-{[(3S)-pyrrolidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(3S)-1-(prop-2-enoyl)pyrrolidin-3-yl]amino}-1H-pyrazolo[3,4-b]pyridin-4-yl)phenyl]methyl}-1,2,4-oxadiazole-3-carboxamide;
10-{[2-fluoro-4-(3-{[(3R)-pyrrolidin-3-yl]amino}-1H-pyrazolo[3,4-b]pyridin-4-yl)phenyl]methyl}-4,4-dimethyl-1,10-diazatricyclo[6.4.0.0²,6]dodeca-2(6),7-dien-9-one;
10-{[2-fluoro-4-(3-{[(3R)-1-(prop-2-enoyl)pyrrolidin-3-yl]amino}-1H-pyrazolo[3,4-b]pyridin-4-yl)phenyl]methyl}-4,4-dimethyl-1,10-diazatricyclo[6.4.0.0²,6]dodeca-2(6),7-dien-9-one;
5-ethyl-N-{[2-fluoro-4-(3-{[(3R)-pyrrolidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(2R,3R)-2-methylpiperidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(3R,6S)-6-methylpiperidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(2R,3R)-2-methyl-1-(prop-2-enoyl)piperidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(3R,6S)-6-methyl-1-(prop-2-enoyl)piperidin-3-yl]amino}-1H-pyrazolo[3,4-b]pyridin-4-yl)phenyl]methyl}-1,2,4-oxadiazole-3-carboxamide;
(3R)-3-({4-[4-({4,4-dimethyl-9-oxo-1,10-diazatricyclo[6.4.0.0²,6]dodeca-2(6),7-dien-10-yl}methyl)-3-fluorophenyl]-1H-pyrazolo[3,4-b]pyridin-3-yl}amino)pyrrolidine-1-carbaldehyde;
(3R)-3-[(4-{4-[(6-tert-butyl-1-oxo-1,2,3,4-tetrahydroisoquinolin-2-yl)methyl]-3-fluorophenyl}-1H-pyrazolo[3,4-b]pyridin-3-yl)amino]pyrrolidine-1-carbaldehyde;
4-tert-butyl-N-{[2-fluoro-4-(3-{[(3S)-1-formylpyrrolidin-3-yl]amino}-1H-pyrazolo[3,4-b]pyridin-4-yl)phenyl]methyl}benzamide;
3-tert-butyl-N-{[2-fluoro-4-(3-{[(3R)-pyrrolidin-3-yl]amino}-1H-pyrazolo[3,4-b]pyridin-4-yl)phenyl]methyl}benzamide;
5-tert-butyl-N-{[2-fluoro-4-(3-{[(3R)-pyrrolidin-3-yl]amino}-1H-pyrazolo[3,4-b]pyridin-4-yl)phenyl]methyl}-1,2-oxazole-3-carboxamide;
1-tert-butyl-N-{[2-fluoro-4-(3-{[(3R)-pyrrolidin-3-yl]amino}-1H-pyrazolo[3,4-b]pyridin-4-yl)phenyl]methyl}-1H-pyrazole-3-carboxamide;
5-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,2-oxazole-3-carboxamide;
1-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1H-pyrazole-3-carboxamide;
(2S,5R)-5-{[4-(4-{[(5-tert-butyl-1,2,4-oxadiazol-3-yl)formamido]methyl}-3-fluorophenyl)-1H-pyrazolo[3,4-b]pyridin-3-yl]amino}-N,N,2-trimethylpiperidine-1-carboxamide;
5-tert-butyl-N-{[2-fluoro-4-(3-{[(3R,6 S)-1-formyl-6-methylpiperidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(3R)-piperidin-3-yl]amino}-1H-pyrazolo[3,4-b]pyridin-4-yl)phenyl]methyl}-1,2,4-oxadiazole-3-carboxamide;
5-tert-butyl-N-{1-[4-(3-{[(3R)-piperidin-3-yl]amino}-1H-pyrazolo[3,4-b]pyridin-4-yl)phenyl]cyclopropyl}-1,2,4-oxadiazole-3-carboxamide;
3-tert-butyl-N-{1-[4-(3-{[(3R)-pyrrolidin-3-yl]amino}-1H-pyrazolo[3,4-b]pyridin-4-yl)phenyl]cyclopropyl}-1,2,4-oxadiazole-5-carboxamide;
5-tert-butyl-N-[(1S)-1-[4-(3-{[(3R)-pyrrolidin-3-yl]amino}-1H-pyrazolo[3,4-b]pyridin-4-yl)phenyl]ethyl]-1,2,4-oxadiazole-3-carboxamide;
5-tert-butyl-N-[(1R)-1-[4-(3-{[(3R)-pyrrolidin-3-yl]amino}-1H-pyrazolo[3,4-b]pyridin-4-yl)phenyl]ethyl]-1,2,4-oxadiazole-3-carboxamide;
5-tert-butyl-N-{[3-fluoro-5-(3-{[(3R)-pyrrolidin-3-yl]amino}-1H-pyrazolo[3,4-b]pyridin-4-yl)phenyl]methyl}-1,2,4-oxadiazole-3-carboxamide;
4-tert-butyl-N-{[3-fluoro-5-(3-{[(3R)-pyrrolidin-3-yl]amino}-1H-pyrazolo[3,4-b]pyridin-4-yl)phenyl]methyl}benzamide;
1-tert-butyl-N-{[2-fluoro-4-(3-{[(3R)-pyrrolidin-3-yl]amino}-1H-pyrazolo[3,4-b]pyridin-4-yl)phenyl]methyl}-1H-pyrazole-4-carboxamide;
5-tert-butyl-N-{[2-fluoro-4-(3-{[(3R)-pyrrolidin-3-yl]amino}-1H-pyrazolo[3,4-b]pyridin-4-yl)phenyl]methyl}-1,3,4-oxadiazole-2-carboxamide;
3-tert-butyl-N-{[2-fluoro-4-(3-{[(3R)-pyrrolidin-3-yl]amino}-1H-pyrazolo[3,4-b]pyridin-4-yl)phenyl]methyl}-1H-1,2,4-triazole-5-carboxamide;
3-tert-butyl-N-{[2-fluoro-4-(3-{[(3R)-pyrrolidin-3-yl]amino}-1H-pyrazolo[3,4-b]pyridin-4-yl)phenyl]methyl}-1,2,4-oxadiazole-5-carboxamide;
1-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1H-pyrazole-4-carboxamide;
5-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,3,4-oxadiazole-2-carboxamide;
3-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1H-1,2,4-triazole-5-carboxamide;
3-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,2,4-oxadiazole-5-carboxamide;
5-ethyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(3R)-1-(prop-2-enoyl)piperidin-3-yl]amino}-1H-pyrazolo[3,4-b]pyridin-4-yl)phenyl]methyl}-1,2,4-oxadiazole-3-carboxamide;
3-tert-butyl-N-{1-[4-(3-{[(3R)-1-(prop-2-enoyl)pyrrolidin-3-yl]amino}-1H-pyrazolo[3,4-b]pyridin-4-yl)phenyl]cyclopropyl}-1,2,4-oxadiazole-5-carboxamide;
5-tert-butyl-N-{2-[2-fluoro-4-(3-{[(3R)-1-(prop-2-enoyl)pyrrolidin-3-yl]amino}-1H-pyrazolo[3,4-b]pyridin-4-yl)phenyl]ethyl}-1,2,4-oxadiazole-3-carboxamide;
5-tert-butyl-N-{[3-fluoro-5-(3-{[(3R)-1-(prop-2-enoyl)pyrrolidin-3-yl]amino}-1H-pyrazolo[3,4-b]pyridin-4-yl)phenyl]methyl}-1,2,4-oxadiazole-3-carboxamide;
4-tert-butyl-N-{[3-fluoro-5-(3-{[(3R)-1-(prop-2-enoyl)pyrrolidin-3-yl]amino}-1H-pyrazolo[3,4-b]pyridin-4-yl)phenyl]methyl}benzamide;
5-tert-butyl-N-{[2-fluoro-4-(3-{[(3R)-1-formylpiperidin-3-yl]amino}-1H-pyrazolo[3,4-b]pyridin-4-yl)phenyl]methyl}-1,2,4-oxadiazole-3-carboxamide;
4-tert-butyl-N-[(1S)-1-[4-(3-{[(3R)-pyrrolidin-3-yl]amino}-1H-pyrazolo[3,4-b]pyridin-4-yl)phenyl]ethyl]benzamide;
N-{[4-(3-{[(3S)-1-acetylpyrrolidin-3-yl]amino}-1H-pyrazolo[3,4-b]pyridin-4-yl)-2-fluorophenyl]methyl}-5-tert-butyl-1,2,4-oxadiazole-3-carboxamide;
5-tert-butyl-N-{[2-fluoro-4-(3-{[(3S)-1-formylpyrrolidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(3S)-1-(3-methyloxetane-3-carbonyl)pyrrolidin-3-yl]amino}-1H-pyrazolo[3,4-b]pyridin-4-yl)phenyl]methyl}-1,2,4-oxadiazole-3-carboxamide;
5-tert-butyl-N-[(1S)-1-[4-(3-{[(3R)-1-(prop-2-enoyl)pyrrolidin-3-yl]amino}-1H-pyrazolo[3,4-b]pyridin-4-yl)phenyl]ethyl]-1,2,4-oxadiazole-3-carboxamide;
5-tert-butyl-N-[(1S)-1-[4-(3-{[(3R)-1-formylpyrrolidin-3-yl]amino}-1H-pyrazolo[3,4-b]pyridin-4-yl)phenyl]ethyl]-1,2,4-oxadiazole-3-carboxamide;
4-tert-butyl-N-[(1S)-1-[4-(3-{[(3R)-1-(prop-2-enoyl)pyrrolidin-3-yl]amino}-1H-pyrazolo[3,4-b]pyridin-4-yl)phenyl]ethyl]benzamide;
5-tert-butyl-N-[(1R)-1-[4-(3-{[(3R)-1-(prop-2-enoyl)pyrrolidin-3-yl]amino}-1H-pyrazolo[3,4-b]pyridin-4-yl)phenyl]ethyl]-1,2,4-oxadiazole-3-carboxamide;
5-tert-butyl-N-[(1R)-1-[4-(3-{[(3R)-1-formylpyrrolidin-3-yl]amino}-1H-pyrazolo[3,4-b]pyridin-4-yl)phenyl]ethyl]-1,2,4-oxadiazole-3-carboxamide;
N-[(4-{3-[(3R)-3-aminopyrrolidin-1-yl]-1H-pyrazolo[3,4-b]pyridin-4-yl}-2-fluorophenyl)methyl]-4-tert-butylbenzamide;
4-tert-butyl-N-[(2-fluoro-4-{3-[(3R)-3-(prop-2-enamido)pyrrolidin-1-yl]-1H-pyrazolo[3,4-b]pyridin-4-yl}phenyl)methyl]benzamide;
5-tert-butyl-N-[(2-fluoro-4-{3-[(3R)-3-(prop-2-enamido)pyrrolidin-1-yl]-1H-pyrazolo[3,4-b]pyridin-4-yl}phenyl)methyl]-1,2,4-oxadiazole-3-carboxamide;
4-tert-butyl-N-[(2-fluoro-4-{3-[(piperidin-4-yl)amino]-1H-pyrazolo[3,4-b]pyridin-4-yl}phenyl)methyl]benzamide;
4-tert-butyl-N-{[2-fluoro-4-(3-{[1-(prop-2-enoyl)piperidin-4-yl]amino}-1H-pyrazolo[3,4-b]pyridin-4-yl)phenyl]methyl}benzamide;
5-tert-butyl-N-{[2-fluoro-4-(3-{[1-(prop-2-enoyl)piperidin-4-yl]amino}-1H-pyrazolo[3,4-b]pyridin-4-yl)phenyl]methyl}-1,2,4-oxadiazole-3-carboxamide;
4-tert-butyl-N-{[2-fluoro-4-(3-{[1-(prop-2-enoyl)azetidin-3-yl]amino}-1H-pyrazolo[3,4-b]pyridin-4-yl)phenyl]methyl}benzamide;
5-tert-butyl-N-{[2-fluoro-4-(3-{[1-(prop-2-enoyl)azetidin-3-yl]amino}-1H-pyrazolo[3,4-b]pyridin-4-yl)phenyl]methyl}-1,2,4-oxadiazole-3-carboxamide;
N-[(4-{3-[(3R)-3-aminopyrrolidin-1-yl]-1H-pyrazolo[3,4-b]pyridin-4-yl}-2-fluorophenyl)methyl]-5-tert-butyl-1,2,4-oxadiazole-3-carboxamide;
5-tert-butyl-N-[(2-fluoro-4-{3-[(piperidin-4-yl)amino]-1H-pyrazolo[3,4-b]pyridin-4-yl}phenyl)methyl]-1,2,4-oxadiazole-3-carboxamide;
N-{[4-(3-{[(3R)-1-(but-2-ynoyl)pyrrolidin-3-yl]amino}-1H-pyrazolo[3,4-b]pyridin-4-yl)-2-fluorophenyl]methyl}-4-(trifluoromethyl)benzamide;
N-{[4-(3-{[(3R)-1-(but-2-ynoyl)pyrrolidin-3-yl]amino}-1H-pyrazolo[3,4-b]pyridin-4-yl)-2-fluorophenyl]methyl}-5-tert-butyl-1,2-oxazole-3-carboxamide;
N-{[4-(3-{[(3R)-1-(but-2-ynoyl)pyrrolidin-3-yl]amino}-1H-pyrazolo[3,4-b]pyridin-4-yl)-2-fluorophenyl]methyl}-4-methoxybenzamide;
5-tert-butyl-N-{[4-(3-{[(3S)-1-(dimethylcarbamoyl)pyrrolidin-3-yl]amino}-1H-pyrazolo[3,4-b]pyridin-4-yl)-2-fluorophenyl]methyl}-1,2,4-oxadiazole-3-carboxamide;
(3S)-3-{[4-(4-{[(4-tert-butylphenyl)formamido]methyl}-3-fluorophenyl)-1H-pyrazolo[3,4-b]pyridin-3-yl]amino}-N,N-dimethylpyrrolidine-1-carb oxamide;
5-tert-butyl-N-{[4-(3-{[(3R)-1-(dimethylcarbamoyl)pyrrolidin-3-yl]amino}-1H-pyrazolo[3,4-b]pyridin-4-yl)-2-fluorophenyl]methyl}-1,2-oxazole-3-carboxamide;
(3R)-3-{[4-(4-{[(5-tert-butyl-1,2,4-oxadiazol-3-yl)formamido]methyl}-3-fluorophenyl)-1H-pyrazolo[3,4-b]pyridin-3-yl]amino}-N,N-dimethylpiperidine-1-carboxamide;
3-tert-butyl-N-{1-[4-(3-{[(3R)-1-(dimethylcarbamoyl)pyrrolidin-3-yl]amino}-1H-pyrazolo[3,4-b]pyridin-4-yl)phenyl]cyclopropyl}-1,2,4-oxadiazole-5-carboxamide;
5-tert-butyl-N-[(1R)-1-[4-(3-{[(3R)-1-(dimethylcarbamoyl)pyrrolidin-3-yl]amino}-1H-pyrazolo[3,4-b]pyridin-4-yl)phenyl]ethyl]-1,2,4-oxadiazole-3-carboxamide;
1-tert-butyl-N-{[4-(3-{[(3R)-1-(dimethylcarbamoyl)pyrrolidin-3-yl]amino}-1H-pyrazolo[3,4-b]pyridin-4-yl)-2-fluorophenyl]methyl}-1H-pyrazole-4-carboxamide;
3-tert-butyl-N-{[4-(3-{[(3R)-1-(dimethylcarbamoyl)pyrrolidin-3-yl]amino}-1H-pyrazolo[3,4-b]pyridin-4-yl)-2-fluorophenyl]methyl}-1,2,4-oxadiazole-5-carboxamide;
5-tert-butyl-N-{1-[2-fluoro-4-(3-{[(3R)-piperidin-3-yl]amino}-1H-pyrazolo[3,4-b]pyridin-4-yl)phenyl]cyclopropyl}-1,2,4-oxadiazole-3-carboxamide;
5-tert-butyl-N-{2-[4-(3-{[(3R)-pyrrolidin-3-yl]amino}-1H-pyrazolo[3,4-b]pyridin-4-yl)phenyl]propan-2-yl}-1,2,4-oxadiazole-3-carboxamide;
N-(5-tert-butyl-1,2,4-oxadiazol-3-yl)-2-[2-fluoro-4-(3-{[(3R)-pyrrolidin-3-yl]amino}-1H-pyrazolo[3,4-b]pyridin-4-yl)phenyl]acetamide;
N-[(4-{3-[(azetidin-3-yl)amino]-1H-pyrazolo[3,4-b]pyridin-4-yl}-2-fluorophenyl)methyl]-5-tert-butyl-1,2,4-oxadiazole-3-carboxamide;
(2R,3R)-3-{[4-(4-{[(5-tert-butyl-1,2,4-oxadiazol-3-yl)formamido]methyl}-3-fluorophenyl)-1H-pyrazolo[3,4-b]pyridin-3-yl]amino}-N,N,2-trimethylpiperidine-1-carboxamide;
5-tert-butyl-N-{[4-(3-{[(2R,3R)-1-cyclopropanecarbonyl-2-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[4-(3-{[(3R,6S)-1-cyclopropanecarbonyl-6-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[4-(3-{[(3R,6S)-1-(cyclopropanesulfonyl)-6-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[3-fluoro-4-(3-{[(3R)-pyrrolidin-3-yl]amino}-1H-pyrazolo[3,4-b]pyridin-4-yl)phenyl]methyl}-1,2,4-oxadiazole-3-carboxamide;
5-tert-butyl-N-[(2-fluoro-4-{3-[(1-propanoylazetidin-3-yl)amino]-1H-pyrazolo[3,4-b]pyridin-4-yl}phenyl)methyl]-1,2,4-oxadiazole-3-carboxamide;
N-{[4-(3-{[1-(but-2-ynoyl)azetidin-3-yl]amino}-1H-pyrazolo[3,4-b]pyridin-4-yl)-2-fluorophenyl]methyl}-5-tert-butyl-1,2,4-oxadiazole-3-carboxamide;
5-tert-butyl-N-[(4-{3-[(3R)-3-[(dimethylcarbamoyl)amino]pyrrolidin-1-yl]-1H-pyrazolo[3,4-b]pyridin-4-yl}-2-fluorophenyl)methyl]-1,2,4-oxadiazole-3-carboxamide;
5-tert-butyl-N-{1-[2-fluoro-4-(3-{[(3R)-1-(prop-2-enoyl)piperidin-3-yl]amino}-1H-pyrazolo[3,4-b]pyridin-4-yl)phenyl]cyclopropyl}-1,2,4-oxadiazole-3-carboxamide;
5-tert-butyl-N-{2-[4-(3-{[(3R)-1-(prop-2-enoyl)pyrrolidin-3-yl]amino}-1H-pyrazolo[3,4-b]pyridin-4-yl)phenyl]propan-2-yl}-1,2,4-oxadiazole-3-carboxamide;
N-(5-tert-butyl-1,2,4-oxadiazol-3-yl)-2-[2-fluoro-4-(3-{[(3R)-1-(prop-2-enoyl)pyrrolidin-3-yl]amino}-1H-pyrazolo[3,4-b]pyridin-4-yl)phenyl]acetamide;
5-tert-butyl-N-{[2-fluoro-4-(3-{[(2R,3R)-2-methyl-1-(pyrrolidine-1-carbonyl)piperidin-3-yl]amino}-1H-pyrazolo[3,4-b]pyridin-4-yl)phenyl]methyl}-1,2,4-oxadiazole-3-carboxamide;
(2R,3R)-3-{[4-(4-{[(5-tert-butyl-1,2,4-oxadiazol-3-yl)formamido]methyl}-3-fluorophenyl)-1H-pyrazolo[3,4-b]pyridin-3-yl]amino}-2-methyl-N-(2,2,2-trifluoroethyl)piperidine-1-carboxamide;
(2S,5R)-5-{[4-(4-{[(5-tert-butyl-1,2,4-oxadiazol-3-yl)formamido]methyl}-3-fluorophenyl)-1H-pyrazolo[3,4-b]pyridin-3-yl]amino}-2-methyl-N-(2,2,2-trifluoroethyl)piperidine-1-carboxamide;
5-tert-butyl-N-{[4-(3-{[(2R,3R)-1-(2-cyanoacetyl)-2-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[4-(3-{[(3R,6S)-1-(2-cyanoacetyl)-6-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[4-(3-{[(2R,3R)-1-(cyclopropanesulfonyl)-2-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[4-(3-{[(2R,3R)-1-(cyanomethyl)-2-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[4-(3-{[(3R,6S)-1-(cyanomethyl)-6-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[2-fluoro-4-(3-{[(3R,6S)-6-methylpiperidin-3-yl]amino}-1H-pyrazolo[3,4-b]pyridin-4-yl)phenyl]methyl}-1,2-oxazole-3-carboxamide;
(2S,5R)-5-{[4-(4-{[(5-tert-butyl-1,2-oxazol-3-yl)formamido]methyl}-3-fluorophenyl)-1H-pyrazolo[3,4-b]pyridin-3-yl]amino}-N,N,2-trimethylpiperidine-1-carboxamide;
5-tert-butyl-N-{[2-fluoro-4-(3-{[(3R,6S)-6-methyl-1-(prop-2-enoyl)piperidin-3-yl]amino}-1H-pyrazolo[3,4-b]pyridin-4-yl)phenyl]methyl}-1,2-oxazole-3-carboxamide;
3-tert-butyl-N-{[2-fluoro-4-(3-{[(3R,6S)-6-methylpiperidin-3-yl]amino}-1H-pyrazolo[3,4-b]pyridin-4-yl)phenyl]methyl}-1,2,4-oxadiazole-5-carboxamide;
3-tert-butyl-N-{[2-fluoro-4-(3-{[(3R,6S)-6-methyl-1-(prop-2-enoyl)piperidin-3-yl]amino}-1H-pyrazolo[3,4-b]pyridin-4-yl)phenyl]methyl}-1,2,4-oxadiazole-5-carboxamide;
N-{[2-fluoro-4-(3-{[(3R)-pyrrolidin-3-yl]amino}-1H-pyrazolo[3,4-b]pyridin-4-yl)phenyl]methyl}-5,5-dimethyl-1H,4H,5H,6H-cyclopenta[b]pyrrole-2-carboxamide;
4-tert-butyl-N-[2-(hydroxymethyl)-3-(3-{[(3R)-pyrrolidin-3-yl]amino}-1H-pyrazolo[3,4-b]pyridin-4-yl)phenyl]benzamide;
5-tert-butyl-N-{[2-fluoro-4-(3-{[(1r,4r)-4-aminocyclohexyl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(1r,4r)-4-(prop-2-enamido)cyclohexyl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(1s,4s)-4-aminocyclohexyl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(1s,4s)-4-(prop-2-enamido)cyclohexyl]amino}-1H-pyrazolo[3,4-b]pyridin-4-yl)phenyl]methyl}-1,2,4-oxadiazole-3-carboxamide;
3-(5-tert-butyl-1,2,4-oxadiazol-3-yl)-1-[2-fluoro-4-(3-{[(3R)-pyrrolidin-3-yl]amino}-1H-pyrazolo[3,4-b]pyridin-4-yl)phenyl]urea;
3-(5-tert-butyl-1,2,4-oxadiazol-3-yl)-1-[2-fluoro-4-(3-{[(3R)-1-(prop-2-enoyl)pyrrolidin-3-yl]amino}-1H-pyrazolo[3,4-b]pyridin-4-yl)phenyl]urea;
N-{[2-fluoro-4-(3-{[(3R)-pyrrolidin-3-yl]amino}-1H-pyrazolo[3,4-b]pyridin-4-yl)phenyl]methyl}-1,5,5-trimethyl-1H,4H,5H,6H-cyclopenta[b]pyrrole-2-carboxamide;
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,5,5-trimethyl-1H,4H,5H,6H-cyclopenta[b]pyrrole-2-carboxamide;
N-[(4-{3-[(3S)-3-aminopyrrolidin-1-yl]-1H-pyrazolo[3,4-b]pyridin-4-yl}-2-fluorophenyl)methyl]-3-tert-butyl-1,2,4-oxadiazole-5-carboxamide;
5-tert-butyl-N-[2-(hydroxymethyl)-3-(3-{[(3R)-1-(prop-2-enoyl)pyrrolidin-3-yl]amino}-1H-pyrazolo[3,4-b]pyridin-4-yl)phenyl]-1,2,4-oxadiazole-3-carboxamide;
4-tert-butyl-N-[2-(hydroxymethyl)-3-(3-{[(3R)-1-(prop-2-enoyl)pyrrolidin-3-yl]amino}-1H-pyrazolo[3,4-b]pyridin-4-yl)phenyl]benzamide;
3-tert-butyl-N-{[2-fluoro-4-(3-{[(3S)-pyrrolidin-3-yl]amino}-1H-pyrazolo[3,4-b]pyridin-4-yl)phenyl]methyl}-1,2,4-oxadiazole-5-carboxamide;
3-tert-butyl-N-{[2-fluoro-4-(3-{[(3S)-1-(propan-2-yl)pyrrolidin-3-yl]amino}-1H-pyrazolo[3,4-b]pyridin-4-yl)phenyl]methyl}-1,2,4-oxadiazole-5-carboxamide;
N-{[4-(3-{[(3S)-1-acetylpyrrolidin-3-yl]amino}-1H-pyrazolo[3,4-b]pyridin-4-yl)-2-fluorophenyl]methyl}-3-tert-butyl-1,2,4-oxadiazole-5-carboxamide;
5-tert-butyl-N-[2-(hydroxymethyl)-3-(3-{[(3R)-pyrrolidin-3-yl]amino}-1H-pyrazolo[3,4-b]pyridin-4-yl)phenyl]-1,2,4-oxadiazole-3-carboxamide;
3-tert-butyl-N-{[2-fluoro-4-(3-{[(3S)-1-methylpyrrolidin-3-yl]amino}-1H-pyrazolo[3,4-b]pyridin-4-yl)phenyl]methyl}-1,2,4-oxadiazole-5-carboxamide;
3-tert-butyl-N-{[2-fluoro-4-(3-{[(3S)-piperidin-3-yl]amino}-1H-pyrazolo[3,4-b]pyridin-4-yl)phenyl]methyl}-1,2,4-oxadiazole-5-carboxamide;
N-{[4-(3-{[(3S)-1-acetylpiperidin-3-yl]amino}-1H-pyrazolo[3,4-b]pyridin-4-yl)-2-fluorophenyl]methyl}-3-tert-butyl-1,2,4-oxadiazole-5-carboxamide;
3-tert-butyl-N-{[2-fluoro-4-(3-{[(3S)-1-(prop-2-enoyl)piperidin-3-yl]amino}-1H-pyrazolo[3,4-b]pyridin-4-yl)phenyl]methyl}-1,2,4-oxadiazole-5-carboxamide;
5-ethyl-N-{[2-fluoro-4-(3-{[(2R,3R)-2-methylpiperidin-3-yl]amino}-1H-pyrazolo[3,4-b]pyridin-4-yl)phenyl]methyl}-1,2-oxazole-3-carboxamide;
(2R,3R)-3-{[4-(4-{[(5-ethyl-1,2-oxazol-3-yl)formamido]methyl}-3-fluorophenyl)-1H-pyrazolo[3,4-b]pyridin-3-yl]amino}-N,N,2-trimethylpiperidine-1-carboxamide;
N-{[2-fluoro-4-(3-{[(2R,3R)-2-methylpiperidin-3-yl]amino}-1H-pyrazolo[3,4-b]pyridin-4-yl)phenyl]methyl}-5-(propan-2-yl)-1,2-oxazole-3-carboxamide;
N-{[2-fluoro-4-(3-{[(2R,3R)-2-methyl-1-(prop-2-enoyl)piperidin-3-yl]amino}-1H-pyrazolo[3,4-b]pyridin-4-yl)phenyl]methyl}-5-(propan-2-yl)-1,2-oxazole-3-carboxamide;
5-tert-butyl-N-{[4-(3-{[(3R)-pyrrolidin-3-yl]amino}-1H-pyrazolo[3,4-b]pyridin-4-yl)phenyl]methyl}-1,2,4-oxadiazole-3-carboxamide;
5-tert-butyl-N-{[4-(3-{[(3R)-1-(prop-2-enoyl)pyrrolidin-3-yl]amino}-1H-pyrazolo[3,4-b]pyridin-4-yl)phenyl]methyl}-1,2,4-oxadiazole-3-carboxamide;
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-2-(propan-2-yl)-1,3-oxazole-5-carboxamide;
2-(dimethylamino)-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,3-oxazole-5-carboxamide;
N-({4-[3-(4-aminopiperidin-1-yl)-1H-pyrazolo[3,4-b]pyridin-4-yl]-2-fluorophenyl}methyl)-3-tert-butyl-1,2,4-oxadiazole-5-carboxamide;
3-tert-butyl-N-[(2-fluoro-4-{3-[4-(prop-2-enamido)piperidin-1-yl]-1H-pyrazolo[3,4-b]pyridin-4-yl}phenyl)methyl]-1,2,4-oxadiazole-5-carboxamide; and
2-tert-butyl-N-{[2-fluoro-4-(3-{[(3R)-pyrrolidin-3-yl]amino}-1H-pyrazolo[3,4-b]pyridin-4-yl)phenyl]methyl}-1,3-oxazole-4-carboxamide;
   or a pharmaceutically acceptable salt thereof.

In certain embodiments, the compound is selected from compounds 181-425, or a pharmaceutically acceptable salt thereof.

At least some of the chemical names of compounds of the invention as given and set forth in this application, may have been generated on an automated basis by use of a commercially available chemical naming software program, and have not been independently verified. Representative programs performing this function include the ChemDraw naming tool sold by Cambridge Software, Inc. and the Instant JChem Software tool sold by ChemAxon, Inc. In the instance where the indicated chemical name and the depicted structure differ, the depicted structure will control.

In another aspect the present invention provides a pharmaceutical composition comprising a therapeutically effective amount of a compound described herein, or a pharmaceutically acceptable salt thereof; or a stereoisomer or an isotopic variant thereof, and a pharmaceutically acceptable excipient. In one embodiment, the pharmaceutical composition comprising the compound described herein, or a pharmaceutically acceptable salt thereof; or a stereoisomer or an isotopic variant thereof, is formulated for a route of administration selected from oral administration, parenteral administration, buccal administration, nasal administration, topical administration, or rectal administration.

In another aspect the present invention provides a method for treating an autoimmune disease or condition comprising administering to a patient in need a therapeutically effective amount of a compound described herein, or a pharmaceutically acceptable salt thereof; or a stereoisomer or an isotopic variant thereof. In one embodiment the autoimmune disease is selected from rheumatoid arthritis or lupus. In a further aspect the present invention provides a method for treating a heteroimmune disease or condition comprising administering to a patient in need a therapeutically effective amount of a compound described herein, or a pharmaceutically acceptable salt thereof; or a stereoisomer or an isotopic variant thereof. In yet another embodiment the present invention provides a method for treating a cancer comprising administering to a patient in need a therapeutically effective amount of a compound described herein, or a pharmaceutically acceptable salt thereof; or a stereoisomer or an isotopic variant thereof. In one embodiment the cancer is a B-cell proliferative disorder. In another embodiment the B-cell proliferative disorder is diffuse large B cell lymphoma, follicular lymphoma, mantle cell lymphoma, or chronic lymphocytic leukemia.

In yet a further aspect the present invention provides a method for treating mastocytosis comprising administering to a patient in need a therapeutically effective amount of a compound described herein, or a pharmaceutically acceptable salt thereof; or a stereoisomer or an isotopic variant thereof.

In another aspect the present invention provides a method for treating osteoporosis or bone resorption disorders comprising administering to a patient in need a therapeutically effective amount of a compound described herein, or a pharmaceutically acceptable salt thereof; or a stereoisomer or an isotopic variant thereof.

In a further aspect the present invention provides a method for treating an inflammatory disease or condition comprising administering to a patient in need a therapeutically effective amount of a compound described herein, or a pharmaceutically acceptable salt thereof; or a stereoisomer or an isotopic variant thereof.

In some embodiments, the compounds described herein reversibly inhibit Btk and in other embodiments are used to treat patients suffering from Bruton's tyrosine kinase-dependent or Bruton's tyrosine kinase mediated conditions or diseases, including, but not limited to, cancer, autoimmune and other inflammatory diseases.

In some embodiments, the compounds described herein irreversibly inhibit Btk and in other embodiments are used to treat patients suffering from Bruton's tyrosine kinase-dependent or Bruton's tyrosine kinase mediated conditions or diseases, including, but not limited to, cancer, autoimmune and other inflammatory diseases.

### Preparation of Compounds

Compounds described herein may be synthesized using standard synthetic reactions known to those of skill in the art or using methods known in the art. The reactions can be employed in a linear sequence to provide the compounds or they may be used to synthesize fragments which are subsequently joined by the methods known in the art.

Described herein are compounds that inhibit the activity of tyrosine kinase(s), such as Btk, and processes for their preparation. Also described herein are pharmaceutically acceptable salts of such compounds. Pharmaceutical compositions that include at least one such compound or a pharmaceutically acceptable salt of such compound, are provided.

The starting material used for the synthesis of the compounds described herein may be synthesized or can be obtained from commercial sources, such as, but not limited to, Aldrich Chemical Co. (Milwaukee, Wis.), Bachem (Torrance, Calif.), or Sigma Chemical Co. (St. Louis, Mo.). The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001); Green and Wuts, PROTECTIVE GROUPS IN ORGANIC SYNTHESIS 3^{rd} Ed., (Wiley 1999); Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); and Larock's Comprehensive Organic Transformations (VCH Publishers Inc., 1989). (all of which are incorporated by reference in their entirety). Other methods for the synthesis of compounds described herein may be found in International Patent Publication No. WO 01/01982901, Arnold et al. Bioorganic & Medicinal Chemistry Letters 10 (2000) 2167-2170; Burchat et al. Bioorganic & Medicinal Chemistry Letters 12 (2002) 1687-1690. General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein.

The products of the reactions may be isolated and purified, if desired, using conventional techniques, including, but not limited to, filtration, distillation, precipitation, chromatography and the like. Such materials may be characterized using conventional means, including physical constants and spectral data.

Compounds described herein may be prepared as a single isomer or a mixture of isomers.

In some aspects, the compounds of Formula (I) are prepared according to following general synthetic scheme Scheme A.

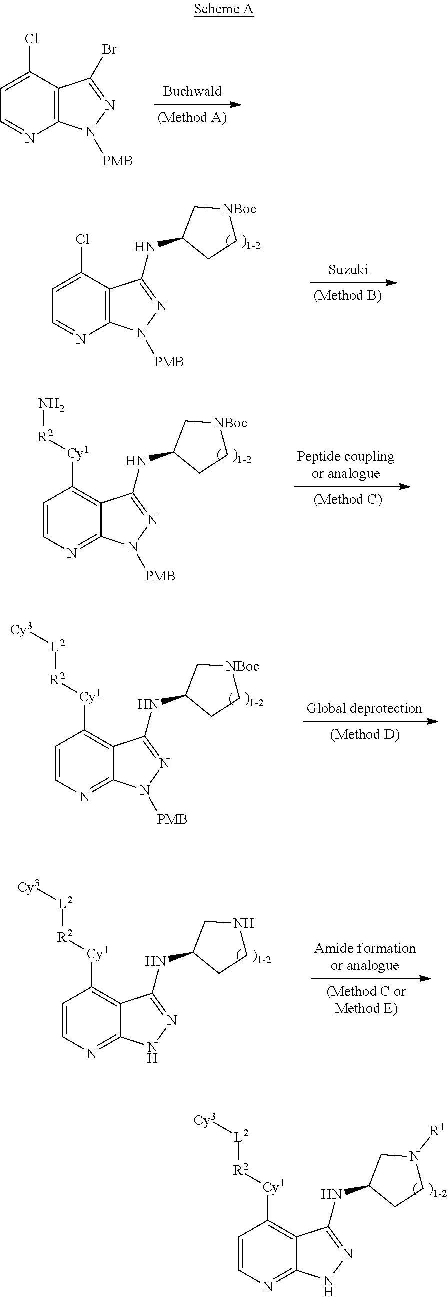

### Further Forms of Compounds

The compounds described herein may possess one or more stereocenters and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

Diasteromeric mixtures can be separated into their individual diastereomers on the basis of their physical chemical differences by methods known, for example, by chromatography and/or fractional crystallization. In one embodiment, enantiomers can be separated by chiral chromatographic columns. In other embodiments, enantiomers can be separated by converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomers, enantiomers, and mixtures thereof are considered as part of the compositions described herein.

The methods and formulations described herein include the use of N-oxides or pharmaceutically acceptable salts of compounds described herein. In some situations, compounds may exist as tautomers. All tautomers are included within the scope of the compounds presented herein.

Compounds as described herein in unoxidized form can be prepared from N-oxides of such compounds as described herein by treating with a reducing agent, such as, but not limited to, sulfur, sulfur dioxide, triphenyl phosphine, lithium borohydride, sodium borohydride, phosphorus trichloride, tribromide, or the like in a suitable inert organic solvent, such as, but not limited to, acetonitrile, ethanol, aqueous dioxane, or the like at about 0 to about 80° C.

Compounds described herein include isotopically-labeled compounds, which are identical to those recited in the various formulas and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays.

Compounds described herein may be formed as, and/or used as, pharmaceutically acceptable salts. The type of pharmaceutical acceptable salts, include, but are not limited to: (1) acid addition salts, formed) by reacting the free base form of the compound with a pharmaceutically acceptable: inorganic acid such as hydrochloric acid, hydrobromic acid, sulfuric acid, nitric acid, phosphoric acid, metaphosphoric acid, and the like; or with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; (2) salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like.

The corresponding counterions of the pharmaceutically acceptable salts may be analyzed and identified using various methods including, but not limited to, ion exchange chromatography, ion chromatography, capillary electrophoresis, inductively coupled plasma, atomic absorption spectroscopy, mass spectrometry, or any combination thereof.

The salts are recovered by using at least one of the following techniques: filtration, precipitation with a non-solvent followed by filtration, evaporation of the solvent, or, in the case of aqueous solutions, lyophilization.

### Pharmaceutical Composition/Formulation

Pharmaceutical compositions may be formulated using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995).

A pharmaceutical composition, as used herein, refers to a mixture of a compound described herein, such as, for example, compounds of any of Formula (I)-(VII), (IIa)-(Va), (IA), (IB), (VIA) or (VIB), with other chemical components, such as carriers, diluents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors.

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, solid dosage forms, tablets, and capsules.

The pharmaceutical compositions will include at least one compound described herein, such as, for example, a compound of any of Formula (I)-(VII), (IIa)-(Va), (IA), (IB), (VIA) or (VIB), as an active ingredient in free-acid or free-base form, or in a pharmaceutically acceptable salt form.

A “carrier” or “carrier materials” include any commonly used excipients in pharmaceutics and should be selected on the basis of compatibility with compounds disclosed herein, such as, compounds of any of Formula (I)-(VII), (IIa)-(Va), (IA), (IB), (VIA) or (VIB).

### Dosage Forms

The compositions described herein can be formulated for administration to a subject via any conventional means including, but not limited to, oral, parenteral (e.g., intravenous, subcutaneous, or intramuscular), buccal, intranasal, rectal or transdermal administration routes. As used herein, the term “subject” is used to mean an animal, preferably a mammal, including a human or non-human. The terms patient and subject may be used interchangeably.

Moreover, the pharmaceutical compositions described herein, which include a compound of any one of Formula (I)-(VII), (IIa)-(Va), (IA), (IB), (VIA) or (VIB), can be formulated into any suitable dosage form for oral ingestion by a patient to be treated.

Pharmaceutical preparations for oral use can be obtained by mixing one or more solid excipient with one or more of the compounds described herein.

All formulations for oral administration should be in dosages suitable for such administration.

In some embodiments, the solid dosage forms disclosed herein may be in the form of a tablet, a pill, or a capsule. In still other embodiments, the pharmaceutical formulation is in the form of a tablet. Additionally, pharmaceutical formulations described herein may be administered as a capsule dosage form.

In some embodiments, solid dosage forms, e.g., tablets, and capsules, are prepared by mixing particles of a compound of any one of Formula (I)-(VII), (IIa)-(Va), (IA), (IB), (VIA) or (VIB) with one or more pharmaceutical excipients.

### Examples of Methods of Dosing and Treatment Regimens

The compounds described herein can be used in the preparation of medicaments for the inhibition of Btk or a homolog thereof, or for the treatment of diseases or conditions that would benefit, at least in part, from inhibition of Btk or a homolog thereof. In addition, a method for treating any of the diseases or conditions described herein in a subject in need of such treatment, involves administration of pharmaceutical compositions containing at least one compound described herein, or a pharmaceutically acceptable salt, stereoisomer, or pharmaceutically acceptable N-oxide, thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

### Combination Treatments

The reversible or irreversible Btk inhibitor compounds and compositions described herein can also be used in combination with other well known therapeutic reagents that are selected for their therapeutic value for the condition to be treated. In general, the compositions described herein and, in embodiments where combinational therapy is employed, other agents do not have to be administered in the same pharmaceutical composition, and may, because of different physical and chemical characteristics, have to be administered by different routes. The initial administration can be made according to established protocols known in the art, and then, based upon the observed effects, the dosage, modes of administration and times of administration can be modified by the skilled clinician.

In certain instances, it may be appropriate to administer at least one reversible or irreversible Btk inhibitor compound described herein in combination with another therapeutic agent. By way of example only, if one of the side effects experienced by a patient upon receiving one of the reversible or irreversible Btk inhibitor compounds described herein is nausea, then it may be appropriate to administer an anti-nausea agent in combination with the initial therapeutic agent. Or, by way of example only, the therapeutic effectiveness of one of the compounds described herein may be enhanced by administration of an adjuvant (i.e., by itself the adjuvant may have minimal therapeutic benefit, but in combination with another therapeutic agent, the overall therapeutic benefit to the patient is enhanced). Alternately, by way of example only, the benefit experienced by a patient may be increased by administering one of the compounds described herein with another therapeutic agent (which also includes a therapeutic regimen) that also has therapeutic benefit. In any case, regardless of the disease, disorder or condition being treated, the overall benefit experienced by the patient may simply be additive of the two therapeutic agents or the patient may experience a synergistic benefit.

The particular choice of compounds used will depend upon the diagnosis of the attending physicians and their judgment of the condition of the patient and the appropriate treatment protocol. The compounds may be administered concurrently (e.g., simultaneously, essentially simultaneously or within the same treatment protocol) or sequentially, depending upon the nature of the disease, disorder, or condition, the condition of the patient, and the actual choice of compounds used. The determination of the order of administration, and the number of repetitions of administration of each therapeutic agent during a treatment protocol, is well within the knowledge of the skilled physician after evaluation of the disease being treated and the condition of the patient.

It is known to those of skill in the art that therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

For combination therapies described herein, dosages of the co-administered compounds will of course vary depending on the type of co-drug employed, on the specific drug employed, on the disease or condition being treated and so forth. In addition, when co-administered with one or more biologically active agents, the compound provided herein may be administered either simultaneously with the biologically active agent(s), or sequentially. If administered sequentially, the attending physician will decide on the appropriate sequence of administering protein in combination with the biologically active agent(s).

It is understood that the dosage regimen to treat or ameliorate the condition(s) for which relief is sought, can be modified in accordance with a variety of factors. These factors include the disorder from which the subject suffers, as well as the age, weight, sex, diet, and medical condition of the subject. Thus, the dosage regimen actually employed can vary widely and therefore can deviate from the dosage regimens set forth herein.

The pharmaceutical agents which make up the combination therapy disclosed herein may be a combined dosage form or in separate dosage forms intended for substantially simultaneous administration

Exemplary Therapeutic Agents for Use in Combination with a Reversible or Irreversible Btk Inhibitor Compound

Where the subject is suffering from or at risk of suffering from an autoimmune disease, an inflammatory disease, or an allergy disease, a reversible or irreversible Btk inhibitor compound can be used in with one or more of the following therapeutic agents in any combination: immunosuppressants (e.g., tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-β, interferon-γ, interleukin-2, allergy vaccines, antihistamines, antileukotrienes, beta-agonists, theophylline, or anticholinergics.

Where the subject is suffering from or at risk of suffering from a B-cell proliferative disorder (e.g., plasma cell myeloma), the subjected can be treated with a reversible or irreversible Btk inhibitor compound in any combination with one or more other anti-cancer agents. In some embodiments, one or more of the anti-cancer agents are proapoptotic agents. Examples of anti-cancer agents include, but are not limited to, any of the following: gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™, such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents for use in combination with a reversible or irreversible Btk inhibitor compound include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors (e.g., everolimus and simrolimus); and antibodies (e.g., rituxan).

Other anti-cancer agents that can be employed in combination with a reversible or irreversible Btk inhibitor compound include adriamycin, dactinomycin, bleomycin, vinblastine, cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or rlL2), interferon α-2a; interferon α-2b; interferon α-n1; interferon α-n3; interferon β-1 a; interferon γ-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer agents that can be employed in combination with a reversible or irreversible Btk inhibitor compound include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that can be employed in combination with a reversible or irreversible Btk inhibitor compound include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, ete.), or triazenes (decarbazine, etc.). Examples of antimetabolites include but are not limited to folic acid analog (e.g., methotrexate), or pyrimidine analogs (e.g., cytarabine), purine analogs (e.g., mercaptopurine, thioguanine, pentostatin).

In some embodiments, the anti-cancer agent is a chemotherapeutic agent, analgesic, an immunotherapeutic agent, a targeted therapy, or a combination thereof. In some embodiments, the additional therapeutic agent is a B cell receptor pathway inhibitor. In some embodiments, the B cell receptor pathway inhibitor is a CD79A inhibitor, a CD79B inhibitor, a CD19 inhibitor, a Lyn inhibitor, a Syk inhibitor, a PI3K inhibitor, a Blnk inhibitor, a PLCγ inhibitor, a PKCβ inhibitor, or a combination thereof. In some embodiments, the additional therapeutic agent is an antibody, B cell receptor signaling inhibitor, a PI3K inhibitor, an IAP inhibitor, an mTOR inhibitor, a radioimmunotherapeutic, a DNA damaging agent, a proteosome inhibitor, a histone deacetylase inhibitor, a protein kinase inhibitor, a hedgehog inhibitor, an Hsp90 inhibitor, a telomerase inhibitor, a Jak1/2 inhibitor, a protease inhibitor, a PKC inhibitor, a PARP inhibitor, or a combination thereof.

In some embodiments, the additional therapeutic agent comprises an analgesic such as acetaminophen.

In some embodiments, the additional therapeutic agent comprises an agent selected from: an inhibitor of LYN, SYK, JAK, PI3K, PLCγ, MAPK, MEK or NFκB.

In some embodiments, the additional therapeutic agent comprises an agent selected from: bendamustine, bortezomib, lenalidomide, idelalisib (GS-1101), vorinostat, everolimus, panobinostat, temsirolimus, romidepsin, vorinostat, fludarabine, cyclophosphamide, mitoxantrone, pentostatine, prednisone, etopside, procarbazine, and thalidomide.

In some embodiments the additional therapeutic agent is bendamustine. In some embodiments, bortezomib is administered in combination with rituximab.

In some embodiments, the additional therapeutic agent is bortezomib. In some embodiments, bendamustine is administered in combination with rituximab.

In some embodiments, the additional therapeutic agent is lenalidomide. In some embodiments, lenalidomide is administered in combination with rituximab.

In some embodiments, the additional therapeutic agent is a multi-agent therapeutic regimen. In some embodiments the additional therapeutic agent comprises the HyperCVAD regimen (cyclophosphamide, vincristine, doxorubicin, dexamethasone alternating with methotrexate and cytarabine). In some embodiments, the HyperCVAD regimen is administered in combination with rituximab.

In some embodiments the additional therapeutic agent comprises the R-CHOP regimen (rituximab, cyclophosphamide, doxorubicin, vincristine, and prednisone).

In some embodiments the additional therapeutic agent comprises the FCR regimen (FCR (fludarabine, cyclophosphamide, rituximab).

In some embodiments the additional therapeutic agent comprises the FCMR regimen (fludarabine, cyclophosphamide, mitoxantrone, rituximab).

In some embodiments the additional therapeutic agent comprises the FMR regimen (fludarabine, mitoxantrone, rituximab).

In some embodiments the additional therapeutic agent comprises the PCR regimen (pentostatin, cyclophosphamide, rituximab).

In some embodiments the additional therapeutic agent comprises the PEPC regimen (prednisone, etoposide, procarbazine, cyclophosphamide).

In some embodiments the additional therapeutic agent comprises radioimmunotherapy with ⁹⁰Y-ibritumomab tiuxetan or ¹³¹I-tositumomab.

In some embodiments, the additional therapeutic agent is an autologous stem cell transplant.

In some embodiments, the additional therapeutic agent is selected from: nitrogen mustards such as for example, bendamustine, chlorambucil, chlormethine, cyclophosphamide, ifosfamide, melphalan, prednimustine, trofosfamide; alkyl sulfonates like busulfan, mannosulfan, treosulfan; ethylene imines like carboquone, thiotepa, triaziquone; nitrosoureas like carmustine, fotemustine, lomustine, nimustine, ranimustine, semustine, streptozocin; epoxides such as for example, etoglucid; other alkylating agents such as for example dacarbazine, mitobronitol, pipobroman, temozolomide; folic acid analogues such as for example methotrexate, permetrexed, pralatrexate, raltitrexed; purine analogs such as for example cladribine, clofarabine, fludarabine, mercaptopurine, nelarabine, tioguanine; pyrimidine analogs such as for example azacitidine, capecitabine, carmofur, cytarabine, decitabine, fluorouracil, gemcitabine, tegafur; vinca alkaloids such as for example vinblastine, vincristine, vindesine, vinflunine, vinorelbine; podophyllotoxin derivatives such as for example etoposide, teniposide; colchicine derivatives such as for example demecolcine; taxanes such as for example docetaxel, paclitaxel, paclitaxel poliglumex; other plant alkaloids and natural products such as for example trabectedin; actinomycines such as for example dactinomycin; antracyclines such as for example aclarubicin, daunorubicin, doxorubicin, epirubicin, idarubicin, mitoxantrone, pirarubicin, valrubicin, zorubincin; other cytotoxic antibiotics such as for example bleomycin, ixabepilone, mitomycin, plicamycin; platinum compounds such as for example carboplatin, cisplatin, oxaliplatin, satraplatin; methylhydrazines such as for example procarbazine; sensitizers such as for example aminolevulinic acid, efaproxiral, methyl aminolevulinate, porfimer sodium, temoporfin; protein kinase inhibitors such as for example dasatinib, erlotinib, everolimus, gefitinib, imatinib, lapatinib, nilotinib, pazonanib, sorafenib, sunitinib, temsirolimus; other antineoplastic agents such as for example alitretinoin, altretamine, amzacrine, anagrelide, arsenic trioxide, asparaginase, bexarotene, bortezomib, celecoxib, denileukin diftitox, estramustine, hydroxycarbamide, irinotecan, lonidamine, masoprocol, miltefosein, mitoguazone, mitotane, oblimersen, pegaspargase, pentostatin, romidepsin, sitimagene ceradenovec, tiazofurine, topotecan, tretinoin, vorinostat; estrogens such as for example diethylstilbenol, ethinylestradiol, fosfestrol, polyestradiol phosphate; progestogens such as for example gestonorone, medroxyprogesterone, megestrol; gonadotropin releasing hormone analogs such as for example buserelin, goserelin, leuprorelin, triptorelin; anti-estrogens such as for example fulvestrant, tamoxifen, toremifene; anti-androgens such as for example bicalutamide, flutamide, nilutamide, enzyme inhibitors, aminoglutethimide, anastrozole, exemestane, formestane, letrozole, vorozole; other hormone antagonists such as for example abarelix, degarelix; immunostimulants such as for example histamine dihydrochloride, mifamurtide, pidotimod, plerixafor, roquinimex, thymopentin; immunosuppressants such as for example everolimus, gusperimus, leflunomide, mycophenolic acid, sirolimus; calcineurin inhibitors such as for example ciclosporin, tacrolimus; other immunosuppressants such as for example azathioprine, lenalidomide, methotrexate, thalidomide; and radiopharmaceuticals such as for example, iobenguane.

In some embodiments, the additional therapeutic agent is selected from: interferons, interleukins, tumor necrosis factors, growth factors, or the like.

In some embodiments, the additional therapeutic agent is selected from: ancestim, filgrastim, lenograstim, molgramostim, pegfilgrastim, sargramostim; interferons such as for example interferon alfa natural, interferon α-2a, interferon α-2b, interferon αcon-1, interferon α-n1, interferon β natural, interferon β-1a, interferon β-1b, interferon γ, peginterferon α-2a, peginterferon α-2b; interleukins such as for example aldesleukin, oprelvekin; other immunostimulants such as for example BCG vaccine, glatiramer acetate, histamine dihydrochloride, immunocyanin, lentinan, melanoma vaccine, mifamurtide, pegademase, pidotimod, plerixafor, poly I:C, poly ICLC, roquinimex, tasonermin, thymopentin; immunosuppressants such as for example abatacept, abetimus, alefacept, antilymphocyte immunoglobulin (horse), antithymocyte immunoglobulin (rabbit), eculizumab, efalizumab, everolimus, gusperimus, leflunomide, muromab-CD3, mycophenolic acid, natalizumab, and sirolimus; TNF α Inhibitors such as for example adalimumab, afelimomab, certolizumab pegol, etanercept, golimumab, infliximab; Interleukin Inhibitors such as for example anakinra, basiliximab, canakinumab, daclizumab, mepolizumab, rilonacept, tocilizumab, and ustekinumab; calcineurin inhibitors such as for example ciclosporin, and tacrolimus; and other immunosuppressants such as for example azathioprine, lenalidomide, methotrexate, and thalidomide.

In some embodiments, the additional therapeutic agent is selected from: adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, panitumumab, ranibizumab, tositumomab, trastuzumab, or the like, or a combination thereof.

In some embodiments, the additional therapeutic agent is selected from: monoclonal antibodies such as for example alemtuzumab, bevacizumab, catumaxomab, cetuximab, edrecolomab, gemtuzumab, panitumumab, trastuzumab; immunosuppressants, eculizumab, efalizumab, muromab-CD3, natalizumab; TNF α inhibitors such as for example adalimumab, afelimomab, certolizumab pegol, golimumab, infliximab; interleukin inhibitors, basiliximab, canakinumab, daclizumab, mepolizumab, tocilizumab, ustekinumab; radiopharmaceuticals, ibritumomab tiuxetan, tositumomab; others monoclonal antibodies such as for example abagovomab, adecatumumab, alemtuzumab, anti-CD30 monoclonal antibody Xmab2513, anti-MET monoclonal antibody MetMab, apolizumab, apomab, arcitumomab, basiliximab, bispecific antibody 2B1, blinatumomab, brentuximab vedotin, capromab pendetide, cixutumumab, claudiximab, conatumumab, dacetuzumab, denosumab, eculizumab, epratuzumab, epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, and zalutumumab.

In some embodiments, the additional therapeutic agent is selected from: agents that affect the tumor micro-environment such as cellular signaling network (e.g. phosphatidylinositol 3-kinase (PI3K) signaling pathway, signaling from the B-cell receptor and the IgE receptor). In some embodiments, the additional therapeutic agent is a PI3K signaling inhibitor or a syc kinase inhibitor. In one embodiment, the syk inhibitor is R788. In another embodiment is a PKCγ inhibitor, such as by way of example only, enzastaurin.

Examples of agents that affect the tumor micro-environment include PI3K signaling inhibitors, syc kinase inhibitors, protein kinase inhibitors such as for example dasatinib, erlotinib, everolimus, gefitinib, imatinib, lapatinib, nilotinib, pazonanib, sorafenib, sunitinib, and temsirolimus; other Angiogenesis Inhibitors such as for example GT-111, JI-101, and R1530; other Kinase Inhibitors such as for example AC220, AC480, ACE-041, AMG 900, AP24534, Arry-614, AT7519, AT9283, AV-951, axitinib, AZD1152, AZD7762, AZD8055, AZD8931, bafetinib, BAY 73-4506, BGJ398, BGT226, BI 811283, BI6727, BIBF 1120, BIBW 2992, BMS-690154, BMS-777607, BMS-863233, BSK-461364, CAL-101, CEP-11981, CYC116, DCC-2036, dinaciclib, dovitinib lactate, E7050, EMD 1214063, ENMD-2076, fostamatinib disodium, GSK2256098, GSK690693, INCB 18424, INNO-406, JNJ-26483327, JX-594, KX2-391, linifanib, LY2603618, MGCD265, MK-0457, MK1496, MLN8054, MLN8237, MP470, NMS-1116354, NMS-1286937, ON 01919.Na, OSI-027, OSI-930, Btk inhibitor, PF-00562271, PF-02341066, PF-03814735, PF-04217903, PF-04554878, PF-04691502, PF-3758309, PHA-739358, PLC3397, progenipoietin, R547, R763, ramucirumab, regorafenib, RO5185426, SAR103168, SCH 727965, SGI-1176, SGX523, SNS-314, TAK-593, TAK-901, TKI258, TLN-232, TTP607, XL147, XL228, XL281RO5126766, XL418, and XL765.

In some embodiments, the additional therapeutic agent is selected from: inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors (e.g., everolimus and simrolimus); and antibodies (e.g., rituxan).

In some embodiments, the additional therapeutic agent is selected from: Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; iimofosine; interleukin Il (including recombinant interleukin II, or rlL2), interferon α-2a; interferon α-2b; interferon α-n1; interferon α-n3; interferon β-1 a; interferon γ-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

In some embodiments, the additional therapeutic agent is selected from: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-such as for example growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In some embodiments, the additional therapeutic agent is selected from: alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, ete.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

In some embodiments, the additional therapeutic agent is selected from: nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, ete.). Examples of antimetabolites include, but are not limited to folic acid analog (e.g., methotrexate), or pyrimidine analogs (e.g., fluorouracil, floxouridine, Cytarabine), purine analogs (e.g., mercaptopurine, thioguanine, pentostatin.

Examples of anti-cancer agents which act by arresting cells in the G2-M phases due to stabilized microtubules and which can be used in combination with a reversible or irreversible Btk inhibitor compound include without limitation the following marketed drugs and drugs in development: e.g., Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCI), AC-7700 (Ajinomoto, also known as AVE-8062, AVE-8062A, CS-39-L-Ser.HCI, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, lnanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Examples of natural products useful in combination with a reversible or irreversible Btk inhibitor compound include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon-α).

Examples of alkylating agents that can be employed in combination a reversible or irreversible Btk inhibitor compound include, but are not limited to, nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, ete.). Examples of antimetabolites include, but are not limited to folic acid analog (e.g., methotrexate), or pyrimidine analogs (e.g., fluorouracil, floxouridine, Cytarabine), purine analogs (e.g., mercaptopurine, thioguanine, pentostatin.

Examples of hormones and antagonists useful in combination with a reversible or irreversible Btk inhibitor compound include, but are not limited to, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

In the instance where the subject is suffering from or at risk of suffering from a thromboembolic disorder (e.g., stroke), the subject can be treated with a reversible or irreversible Btk inhibitor compound in any combination with one or more other anti-thromboembolic agents. Examples of anti-thromboembolic agents include, but are not limited any of the following: thrombolytic agents (e.g., alteplase anistreplase, streptokinase, urokinase, or tissue plasminogen activator), heparin, tinzaparin, warfarin, dabigatran (e.g., dabigatran etexilate), factor Xa inhibitors (e.g., fondaparinux, draparinux, rivaroxaban, DX-9065a, otamixaban, LY517717, or YM150), ticlopidine, clopidogrel, CS-747 (prasugrel, LY640315), ximelagatran, or BIBR 1048.

In some embodiments, the additional anti-cancer agent that can be used in combination with the compounds described herein is a Bcl-2 inhibitor.

In some embodiments, the additional anti-cancer agent is an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collagenous structure), PS (phosphatidylserine), OX-40, SLAM, TIGHT, VISTA, VTCN1, or any combinations thereof. In some embodiments, the immune checkpoint inhibitor is an inhibitor of PD-L1, PD-1, CTLA-4, LAG3, or TIM3. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the immune checkpoint inhibitor is an inhibitor of PD-L2.

In some embodiments, a compound described herein is administered in combination with a CD20 inhibitor. Exemplary CD20 inhibitors include, but are not limited to, ibritumomab tiuxetan, ofatumumab, rituximab, tositumomab, and obinutuzumab.

In some embodiments, the additional anticancer agents used in combination with the compounds described herein include CDK4 inhibitors (e.g., palbociclib).

In some embodiments, the additional cancer agent is a proteosome inhibitor. In some embodiments, the proteasome inhibitor is selected from bortezomib or carfilzomib.

In some embodiments, the additional cancer agent that can be administered in combination with the compounds is an HDAC inhibitor. In some embodiments, the HDAC inhibitor is abexinostat or a salt thereof. In some embodiments, the abexinostat or a salt thereof is abexinostat HCl. In some embodiments, the abexinostat or a salt thereof is abexinostat tosylate.

In some embodiments, the additional cancer agent that can be administered in combination with the compounds is a MALT1 inhibitor, MCL-1 inhibitor, IDH1 inhibitor, TLR inhibitor, or PIM inhibitor.

In some embodiments, the additional anti-cancer agent that can be administered in combination with the compounds is an immunomodulatory agent. Exemplary immunomodulatory agents include, but are not limited to, lenalidomide, thalidomide, and pomalidomide.

### Kits/Articles of Manufacture

For use in the therapeutic applications described herein, kits and articles of manufacture are also described herein. Such kits can include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease, disorder, or condition that would benefit by inhibition of Btk, or in which Btk is a mediator or contributor to the symptoms or cause.

For example, the container(s) can include one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label, or instructions relating to its use in the methods described herein.

A kit will typically may include one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, the pharmaceutical compositions can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutical carrier can also be prepared, placed in an appropriate container, and labeled for treatment of an indicated condition.

### EXAMPLES

The following specific and non-limiting examples are to be construed as merely illustrative, and do not limit the present disclosure in any way whatsoever. Without further elaboration, it is believed that one skilled in the art can, based on the description herein, utilize the present disclosure to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

The examples below as well as throughout the application, the following abbreviations have the following meanings. If not defined, the terms have their generally accepted meanings.
aq=aqueous
[(t-Bu)₃PH]BF₄=tri-tert-butylphosphonium tetrafluoroborate
t-BuOH=tertiary butanol
DCE=1,2-dichloroethane
DCM=dichloromethane
DIEA or DIPEA=N,N-diisopropylethylamine
DMAP=dimethylaminopyridine
DMF=dimethylformamide
DMSO=dimethylsulfoxide
ESI=electron spray ionization
EtOAc and EA=ethyl acetate
g=gram
HCl=hydrogen chloride
HPLC=high performance liquid chromatography
¹H NMR=proton nuclear magnetic resonance
HDNIB hydroxy(2,4-dinitrobenzenesulfonyloxy)iodobenzene
IPA=isopropyl alcohol
LC-MS=liquid chromatography mass spectroscopy
M=molar
MeCN=acetonitrile
MeOH=methanol
mg=milligram
min=minute
mL=milliliter
mM=millimolar
mmol=millimole
m.p.=melting point
MS=mass spectrometry
m/z=mass-to-charge ratio
N=normal
nM=nanomolar
nm=nanometer
Pd₂dba₃=tris(dibenzylideneacetone)dipalladium(0)
p.s.i.=pound per square inch
RT=room temperature
TEA=triethylamine
TFA=trifluoroacetic acid
TLC=thin layer chromatography
μL=microliter
μM=Micromolar
PyBop=Benzotriazol-1-yloxy)tripyrrolidinophosphonium hexafluorophosphate
HATU=1-[Bis(dimethylamino)methylene]-1H-1,2,3-triazolo[4,5-b]pyridinium 3-oxid hexafluorophosphate Pd(dppf)
TBAB=Tetra-n-butylammonium bromide
THF=Tetrahydrofuran
ACN=Acetonitrile

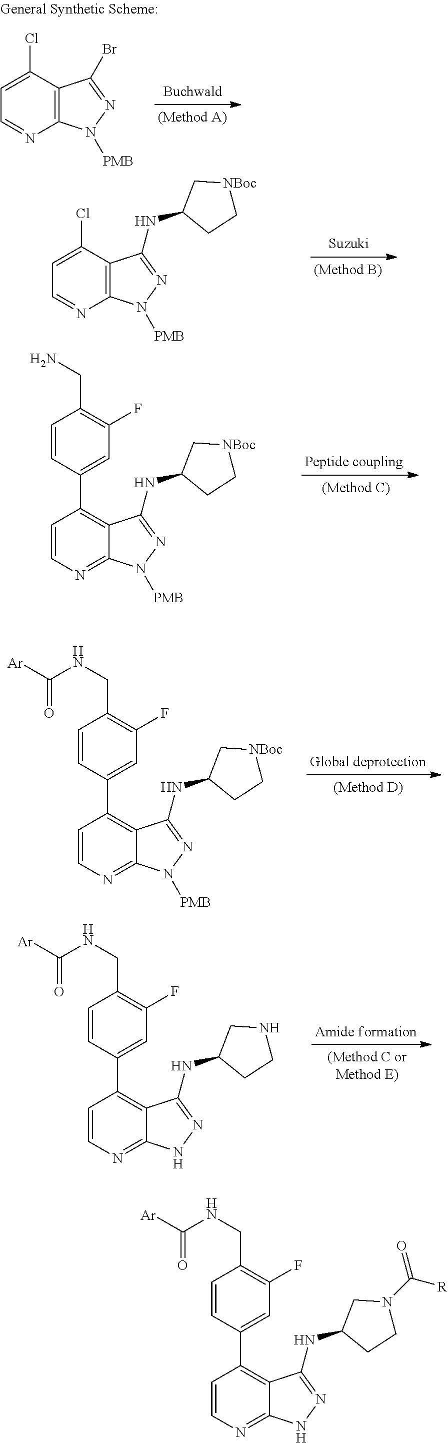

### Example 1: Synthesis of (R)-5-(tert-butyl)-N-(2-fluoro-4-(3-(pyrrolidin-3-ylamino)-1H-pyrazolo[3,4-b]pyridin-4-yl)benzyl)-1,2,4-oxadiazole-3-carboxamide (Compound-2)

### Method A (3.1): Preparation of tert-butyl (R)-3-((4-chloro-1-(4-methoxybenzyl)-1H-pyrazolo[3,4-b]pyridin-3-yl)amino)pyrrolidine-1-carboxylate (Buchwald Reaction)

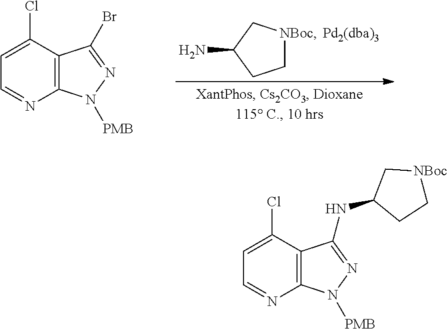

The mixture of 3-bromo-4-chloro-1-(4-methoxybenzyl)-1H-pyrazolo[3,4-b]pyridine (4 g, 12.3 mmol), (R)-tert-butyl 3-aminopiperidine-1-carboxylate (2.74 g, 16.0 mmol), Pd₂(dba)₃ (500 mg, 0.60 mmol), Xantphos (984 mg, 1.84 mmol) and cesium carbonate (5.54 g, 18.4 mmol) in dioxane, was degassed by bubbling with N₂ for 5 min, in a glass bomb. The mixture was then heated at 115° C. for 10 hrs, cooled to room temperature, and partitioned with ethyl acetate (200 ml) and water (50 ml). The aqueous was re-extracted 1× with EA and the organics dried (Na₂SO₄), filtered and evaporated. The crude was purified by flash chromatography (0-50% EA/hexanes as eluent) to afford the title compound (3.41 g, 61%); MH⁺=458.1.

### Method B (3.2): Preparation of tert-butyl (R)-3-((4-(4-(aminomethyl)-3-fluorophenyl)-1-(4-methoxybenzyl)-1H-pyrazolo[3,4-b]pyridin-3-yl)amino)pyrrolidine-1-carboxylate (Suzuki Reaction)

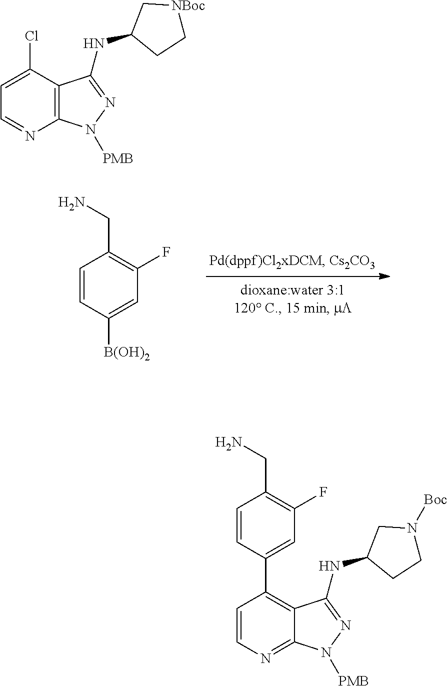

The mixture of tert-butyl (R)-3-((4-chloro-1-(4-methoxybenzyl)-1H-pyrazolo[3,4-b]pyridin-3-yl)amino)pyrrolidine-1-carboxylate (788 mg, 1.72 mmol), (4-(aminomethyl)-3-fluorophenyl)boronic acid (HCl) (608 mg, 2.84 mmol), cesium carbonate (1.26 g, 3.87 mmol), and Pd(dppf)Cl₂xDCM (70 mg, 0.09 mmol) in dioxane (7.5 ml) and water (2.5 ml), was degassed by bubbling N₂ through for 3 min, inside a microwave vial. The mixture was submitted to 120° C. for 15 min in a microwave reactor, then partitioned between ethyl acetate (150 ml) and water:brine (50 ml). The original aqueous was re-extracted 1× with EA and the combined organics dried (Na₂SO₄), filtered, concentrated, and subjected to flash chromatography (0-20% MeOH/DCM as eluent) to give the title compound (882 mg, 94%); MH⁺=547.3.

### Method C (3.4): Preparation of tert-butyl (R)-3-((4-(4-((5-(tert-butyl)-1,2,4-oxadiazole-3-carboxamido)methyl)-3-fluorophenyl)-1-(4-methoxybenzyl)-1H-pyrazolo[3,4-b]pyridin-3-yl)amino)pyrrolidine-1-carboxylate (Typical Pybop Peptide Coupling)

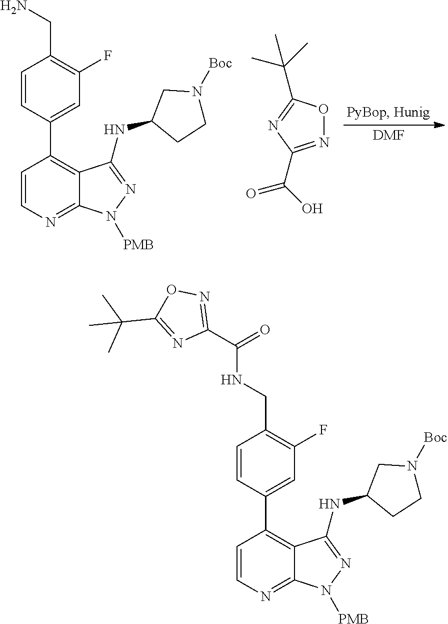

A solution of tert-butyl (R)-3-((4-(4-(aminomethyl)-3-fluorophenyl)-1-(4-methoxybenzyl)-1H-pyrazolo[3,4-b]pyridin-3-yl)amino)pyrrolidine-1-carboxylate (273 mg, 0.5 mmol), 5-(tert-butyl)-1,2,4-oxadiazole-3-carboxylic acid (128 mg, 0.75 mmol), Pybop (416 mg, 0.80 mmol), in DMF (3 ml) and Hunig's base (0.26 ml, 1.5 mmol), was stirred for 2-16 hrs. The mixture was extracted in ethyl acetate (80 ml) and water:NaHCO₃ (20 ml) and the aqueous re-extracted 1×. The combined organics were dried (Na₂SO₄), filtered, concentrated, and purified by flash chromatography (0-65% EtOAc/hexanes as eluent) to give the desired compound (349 mg); MW=698.80, MH⁺=699.4.

### Method D (3.6): Preparation of (R)-5-(tert-butyl)-N-(2-fluoro-4-(3-(pyrrolidin-3-ylamino)-1H-pyrazolo[3,4-b]pyridin-4-yl)benzyl)-1,2,4-oxadiazole-3-carboxamide (Compound-2)

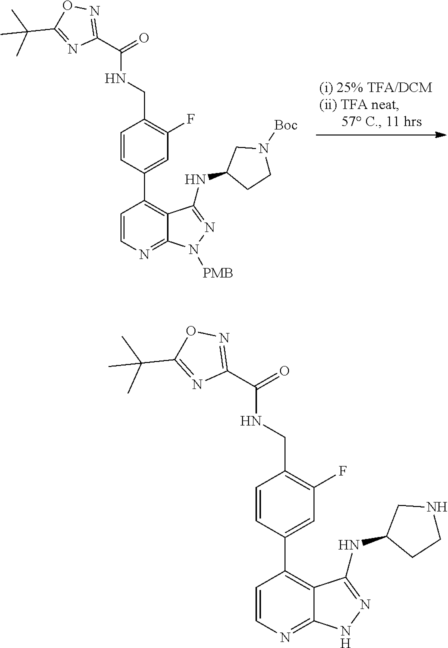

tert-butyl (R)-3-((4-(4-((5-(tert-butyl)-1,2,4-oxadiazole-3-carboxamido)methyl)-3-fluorophenyl)-1-(4-methoxybenzyl)-1H-pyrazolo[3,4-b]pyridin-3-yl)amino)pyrrolidine-1-carboxylate (349 mg (0.5 mmol) was treated with 3 ml of 25% TFA/DCM for 1 hr, then concentrated. The residue was dissolved in 4 ml neat TFA and heated at 57° C. for 11 hrs, then concentrated, diluted with DCM, treated with 0.25 ml TEA, then concentrated again. Upon flash chromatography (0-30% MeOH/DCM as eluent), the title compound was isolated (221 mg, 93%); MH⁺=479.2.

The following compounds were prepared in a similar way:
(R)—N-(2-fluoro-4-(3-(pyrrolidin-3-ylamino)-1H-pyrazolo[3,4-b]pyridin-4-yl)benzyl)benzamide (Compound-31)

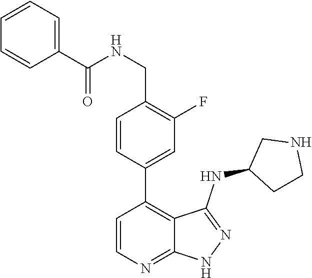 [SMILES: O=C(NCc1ccc(-c2ccnc3[nH]nc(N[C@@H]4CCNC4)c23)cc1F)c1ccccc1]

free-base; MH⁺=431.1.
(R)—N-(2-fluoro-4-(3-(pyrrolidin-3-ylamino)-1H-pyrazolo[3,4-b]pyridin-4-yl)benzyl)-4-methylbenzamide (Compound-32)

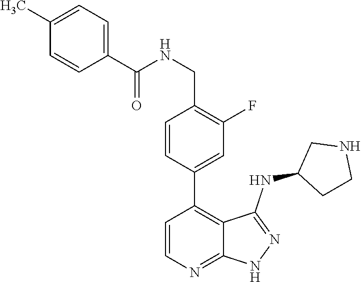 [SMILES: Cc1ccc(C(=O)NCc2ccc(-c3ccnc4[nH]nc(N[C@@H]5CCNC5)c34)cc2F)cc1]

free-base; MH⁺=445.1.

### Example 2: Synthesis of (R)—N-(4-(3-((1-acryloylpyrrolidin-3-yl)amino)-1H-pyrazolo[3,4-b]pyridin-4-yl)-2-fluorobenzyl)-5-(tert-butyl)-1,2,4-oxadiazole-3-carboxamide (Compound-3)

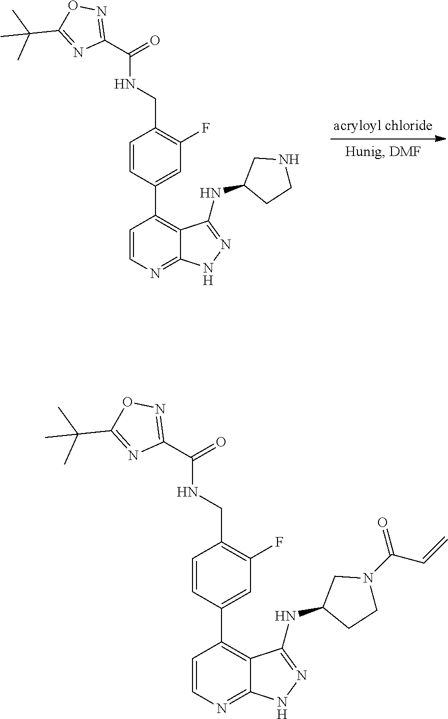 [SMILES: CC(C)(C)c1nc(C(=O)NCc2ccc(-c3ccnc4[nH]nc(N[C@@H]5CCNC5)c34)cc2F)no1 ; C=CC(=O)N1CC[C@@H](Nc2n[nH]c3nccc(-c4ccc(CNC(=O)c5noc(C(C)(C)C)n5)c(F)c4)c23)C1]

### Method E

To a solution of (R)-5-(tert-butyl)-N-(2-fluoro-4-(3-(pyrrolidin-3-ylamino)-1H-pyrazolo[3,4-b]pyridin-4-yl)benzyl)-1,2,4-oxadiazole-3-carboxamide (91 mg, 0.19 mmol) in DMF (2 ml), was added Hunig's base (99 μL, 0.57 mmol), followed by acryloyl chloride (15.4 μL, 0.19 mmol). The solution was stirred for 5 min, then quenched with water, diluted with acetonitrile and water and subjected to reverse phase prep HPLC (using 0.1% formic acid aqueous solution and acetonitrile as mobile phase) to yield the product fractions which were desalted by passing through a bicarbonate resin (500 mg) tube. Free-base product was obtained (46.0 mg, 45%), MH⁺=533.2, M⁺−1=531.2, UV: λ=238, 353 nm.

¹H NMR (400 MHz, DMSO-d6) δ 12.26 (s, 1H), 9.08 (s, 1H), 8.39 (d, 1H, J=4 Hz), 7.68 (m, 1H), 7.51 (m, 1H), 7.37 (m, 1H), 6.93 (d, J=4.0 Hz, 1H), 6.44 (m, 1H), 6.05 (dd, J1=16.8, J2=4 Hz, 1H), 5.56 (m, 1H), 4.59 (d, J=6.4 Hz, 2H), 4.11 (m, 1H), 3.85-3.2 (m, 4H), 2.12 (m, 1H), 1.79 (m, 1H), 1.42 (s, 9H).

The following compounds were prepared in a similar way:
(R)—N-(4-(3-((1-acryloylpyrrolidin-3-yl)amino)-1H-pyrazolo[3,4-b]pyridin-4-yl)-2-fluorobenzyl)benzamide (Compound-33)

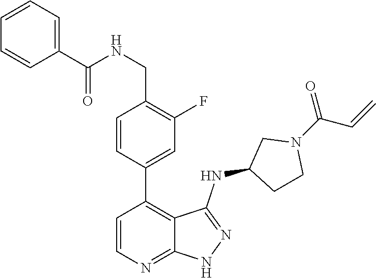 [SMILES: C=CC(=O)N1CC[C@@H](Nc2n[nH]c3nccc(-c4ccc(CNC(=O)c5ccccc5)c(F)c4)c23)C1]

free-base; MH⁺=485.1.
(R)—N-(4-(3-((1-acryloylpyrrolidin-3-yl)amino)-1H-pyrazolo[3,4-b]pyridin-4-yl)-2-fluorobenzyl)-4-methylbenzamide (Compound-34)

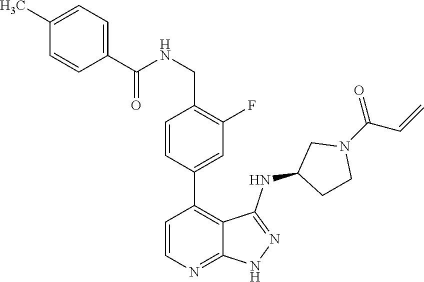 [SMILES: C=CC(=O)N1CC[C@@H](Nc2n[nH]c3nccc(-c4ccc(CNC(=O)c5ccc(C)cc5)c(F)c4)c23)C1]

free-base; MH⁺=499.2, M+−1=497.1, UV: λ=352 nm.
(S)—N-(4-(3-((1-acryloylpyrrolidin-3-yl)amino)-1H-pyrazolo[3,4-b]pyridin-4-yl)-2-fluorobenzyl)-4-(tert-butyl)benzamide (Compound-44)

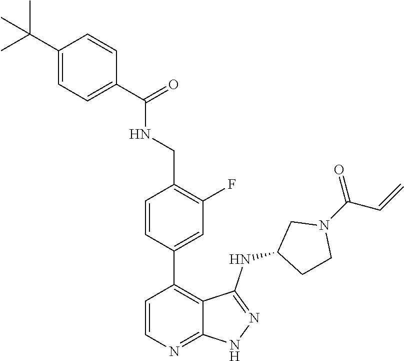 [SMILES: C=CC(=O)N1CC[C@H](Nc2n[nH]c3nccc(-c4ccc(CNC(=O)c5ccc(C(C)(C)C)cc5)c(F)c4)c23)C1]

free-base; MH⁺=541.2.
(R)—N-(4-(3-((1-acryloylpyrrolidin-3-yl)amino)-1H-pyrazolo[3,4-b]pyridin-4-yl)-2-fluorobenzyl)-3-(tert-butyl)benzamide (Compound-46)

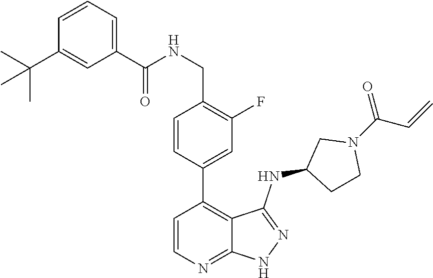 [SMILES: C=CC(=O)N1CC[C@@H](Nc2n[nH]c3nccc(-c4ccc(CNC(=O)c5cccc(C(C)(C)C)c5)c(F)c4)c23)C1]

(R)—N-(4-(3-((1-acryloylpyrrolidin-3-yl)amino)-1H-pyrazolo[3,4-b]pyridin-4-yl)-2-fluorobenzyl)-4-(prop-1-en-2-yl)benzamide (Compound-47)

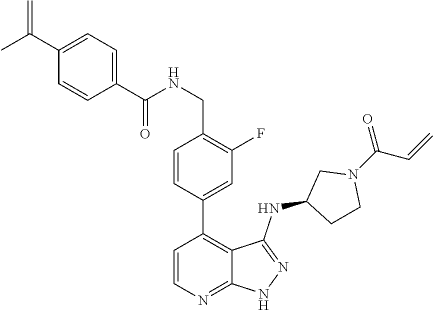 [SMILES: C=CC(=O)N1CC[C@@H](Nc2n[nH]c3nccc(-c4ccc(CNC(=O)c5ccc(C(=C)C)cc5)c(F)c4)c23)C1]

free-base; MH⁺=525.2.
(R)—N-(4-(3-((1-acryloylpyrrolidin-3-yl)amino)-1H-pyrazolo[3,4-b]pyridin-4-yl)-2-fluorobenzyl)-4-(trifluoromethyl)benzamide (Compound-48)

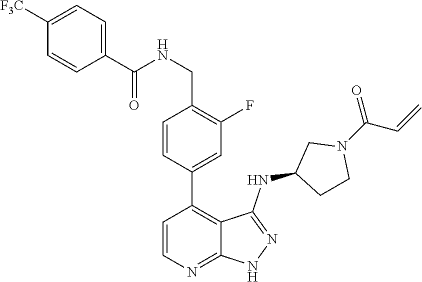 [SMILES: C=CC(=O)N1CC[C@@H](Nc2n[nH]c3nccc(-c4ccc(CNC(=O)c5ccc(C(F)(F)F)cc5)c(F)c4)c23)C1]

free-base; MH⁺=553.2.
(R)—N-(4-(3-((1-acryloylpyrrolidin-3-yl)amino)-1H-pyrazolo[3,4-b]pyridin-4-yl)-2-fluorobenzyl)-4-methoxybenzamide (Compound-49)

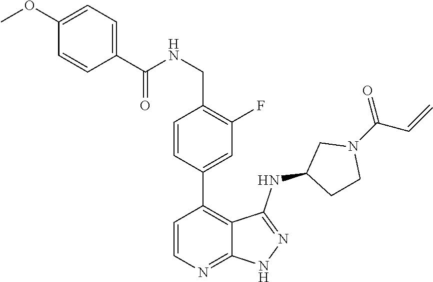 [SMILES: C=CC(=O)N1CC[C@@H](Nc2n[nH]c3nccc(-c4ccc(CNC(=O)c5ccc(OC)cc5)c(F)c4)c23)C1]

free-base; MH⁺=515.1.

### Example 3: Synthesis of 5-tert-butyl-N-{1-[4-(3-{[(3R)-pyrrolidin-3-yl]amino}-1H-pyrazolo[3,4-b]pyridin-4-yl)phenyl]cyclopropyl}-1,2,4-oxadiazole-3-carboxamide (Compound-1)

### Method 3.1. Preparation of tert-butyl (R)-3-((4-chloro-1-(4-methoxybenzyl)-1H-pyrazolo[3,4-b]pyridin-3-yl)amino)pyrrolidine-1-carboxylate (Buchwald Reaction)

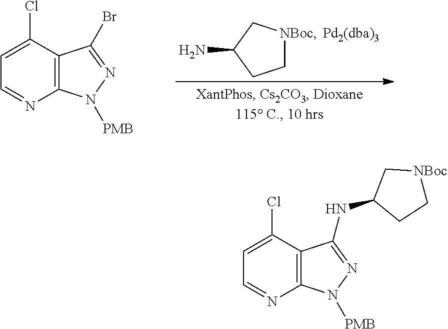

The mixture of 3-bromo-4-chloro-1-(4-methoxybenzyl)-1H-pyrazolo[3,4-b]pyridine (4 g, 12.3 mmol), (R)-tert-butyl 3-aminopiperidine-1-carboxylate (2.74 g, 16.0 mmol), Pd₂(dba)₃ (500 mg, 0.60 mmol), Xantphos (984 mg, 1.84 mmol) and cesium carbonate (5.54 g, 18.4 mmol) in dioxane, was degassed by bubbling with N₂ for 5 min, in a glass bomb. The mixture was then heated at 115° C. for 10 hrs, cooled to room temperature, and partitioned with ethyl acetate (200 ml) and water (50 ml). The aqueous was re-extracted 1× with EA and the organics dried (Na₂SO₄), filtered and evaporated. The crude was purified by flash chromatography (0-50% EA/hexanes as eluent) to afford the title compound (3.41 g, 61%); MH⁺=458.1.

### Method 3.3. Preparation of tert-butyl (R)-3-((4-(4-(1-aminocyclopropyl)phenyl)-1-(4-methoxybenzyl)-1H-pyrazolo[3,4-b]pyridin-3-yl)amino)pyrrolidine-1-carboxylate

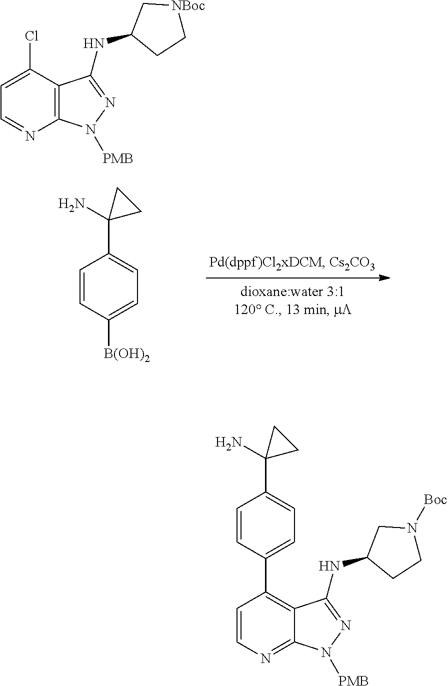

A procedure similar to Method B was used, except (4-(1-aminocyclopropyl)phenyl)boronic acid (HCl) used. Obtained quantitative yield of the desired product; MH⁺=555.3.

### Method 3.5. Preparation of tert-butyl (R)-3-((4-(4-(1-(5-(tert-butyl)-1,2,4-oxadiazole-3-carboxamido)cyclopropyl)phenyl)-1-(4-methoxybenzyl)-1H-pyrazolo[3,4-b]pyridin-3-yl)amino)pyrrolidine-1-carboxylate

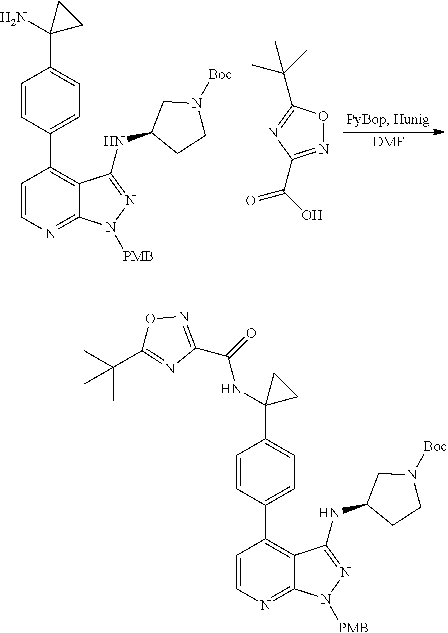

A procedure similar to Method C was used. Obtained the title compound (258 mg, 65%); MH⁺=707.3.

### Method 3.7. Preparation of (R)-5-(tert-butyl)-N-(1-(4-(3-(pyrrolidin-3-ylamino)-1H-pyrazolo[3,4-b]pyridin-4-yl)phenyl)cyclopropyl)-1,2,4-oxadiazole-3-carboxamide (Compound-1)

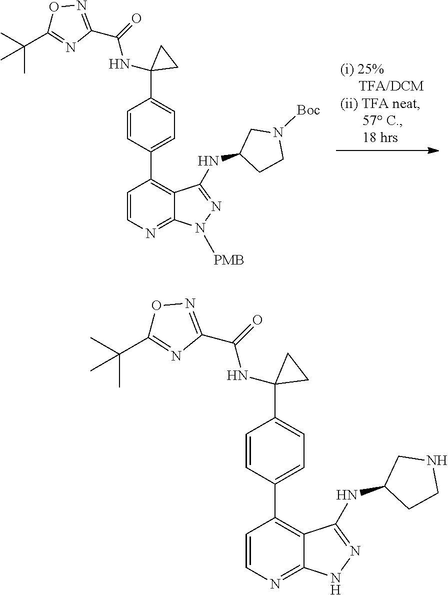

A procedure similar to Method D was used, yielding the title compound (174 mg, 98%); MH⁺=487.2.

The following compound was prepared in a similar way:
(R)—N-(1-(4-(3-((1-acryloylpyrrolidin-3-yl)amino)-1H-pyrazolo[3,4-b]pyridin-4-yl)phenyl)cyclopropyl)-5-(tert-butyl)-1,2,4-oxadiazole-3-carboxamide (Compound-4)

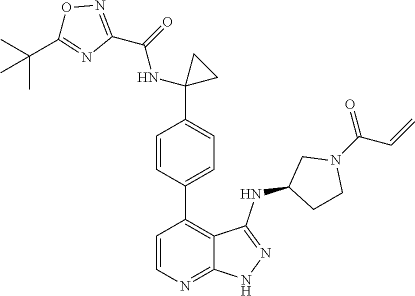 [SMILES: C=CC(=O)N1CC[C@@H](Nc2n[nH]c3nccc(-c4ccc(C5(NC(=O)c6noc(C(C)(C)C)n6)CC5)cc4)c23)C1]

free-base; MH⁺=541.3. ¹H NMR (400 MHz, DMSO-d6) δ 12.18 (s, 1H), 9.31 (s, 1H), 8.37 (d, 1H, J=4 Hz), 7.89 (m, 1H), 7.43 (m, 3H), 6.88 (d, J=4.0 Hz, 1H), 6.46 (m, 1H), 6.06 (m, 1H), 5.57 (dd, J1=2.5 Hz, J2=10.6 Hz, 1H), 4.14 (m, 1H), 3.93 (m, 1H), 3.8-3.2 (m, 4H), 2.09 (m, 1H), 1.73 (m, 1H), 1.42 (s, 9H), 1.38-1.30 (m, 4H).

### Example 4: Synthesis of (R)-5-(tert-butyl)-N-(1-(4-(3-((1-propionylpyrrolidin-3-yl)amino)-1H-pyrazolo[3,4-b]pyridin-4-yl)phenyl)cyclopropyl)-1,2,4-oxadiazole-3-carboxamide (Final Pybop peptide coupling) (Compound-5)

### Method 3.9. Preparation of (R)-5-(tert-butyl)-N-(1-(4-(3-((1-propionylpyrrolidin-3-yl)amino)-1H-pyrazolo[3,4-b]pyridin-4-yl)phenyl)cyclopropyl)-1,2,4-oxadiazole-3-carboxamide (Final Pybop peptide coupling)

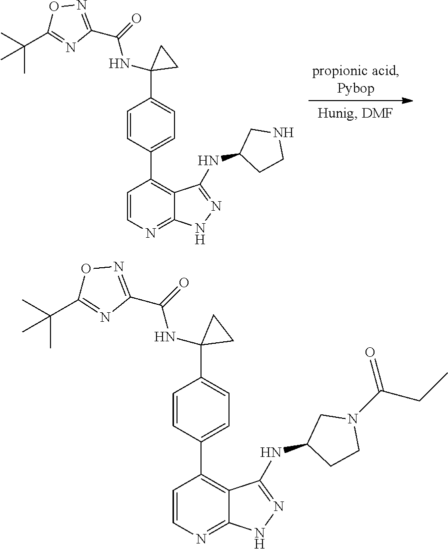 [SMILES: CC(C)(C)c1nc(C(=O)NC2(c3ccc(-c4ccnc5[nH]nc(N[C@@H]6CCNC6)c45)cc3)CC2)no1 ; CCC(=O)N1CC[C@@H](Nc2n[nH]c3nccc(-c4ccc(C5(NC(=O)c6noc(C(C)(C)C)n6)CC5)cc4)c23)C1]

To a solution of (R)-5-(tert-butyl)-N-(1-(4-(3-(pyrrolidin-3-ylamino)-1H-pyrazolo[3,4-b]pyridin-4-yl)phenyl)cyclopropyl)-1,2,4-oxadiazole-3-carboxamide (16.5 mg, 0.034 mmol) in DMF (0.5 ml), was added: Hunig's base (18 μL, 0.102 mmol), Pybop (26.5 mg, 0.051 mmol) and propionic acid (3.8 μL, 0.051 mmol). The mixture was stirred for 40 min, then quenched with water, diluted further with acetonitrile and water and subjected to reverse phase prep HPLC (using 0.1% formic acid aqueous solution and acetonitrile as mobile phase) to yield the product fractions which were desalted by passing through a bicarbonate resin (500 mg) tube. Free-base product was obtained (8.7 mg, 47%), MH⁺=543.2. ¹H NMR (400 MHz, DMSO-d6) δ 12.16 (s, 1H), 9.32 (s, 1H), 8.37 (d, 1H, J=5 Hz), 7.87 (m, 1H), 7.45 (m, 3H), 6.88 (d, J=5.0 Hz, 1H), 4.10 (m, 1H), 3.90 (m, 1H), 3.7-3.14 (m, 4H), 2.14 (m, 2H), 1.66 (m, 2H), 1.42 (s, 9H), 1.39-1.30 (m, 4H), 0.96 (t, J=8.2 Hz).

The following compounds were prepared in a similar way:
(R)—N-(1-(4-(3-((1-(but-2-ynoyl)pyrrolidin-3-yl)amino)-1H-pyrazolo[3,4-b]pyridin-4-yl)phenyl)cyclopropyl)-5-(tert-butyl)-1,2,4-oxadiazole-3-carboxamide (Compound-28)

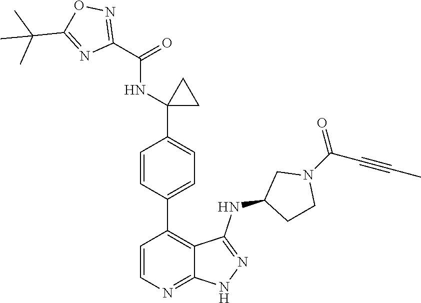 [SMILES: CC#CC(=O)N1CC[C@@H](Nc2n[nH]c3nccc(-c4ccc(C5(NC(=O)c6noc(C(C)(C)C)n6)CC5)cc4)c23)C1]

free-base; MH⁺=553.2.
(R,E)-5-(tert-butyl)-N-(1-(4-(3-((1-(4-(dimethylamino)but-2-enoyl)pyrrolidin-3-yl)amino)-1H-pyrazolo[3,4-b]pyridin-4-yl)phenyl)cyclopropyl)-1,2,4-oxadiazole-3-carboxamide (Compound-29)

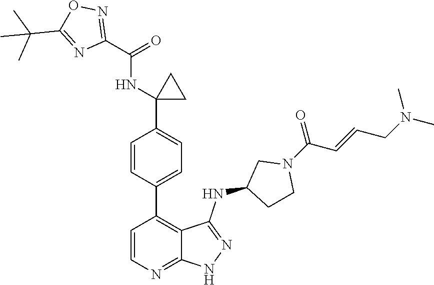 [SMILES: CN(C)C/C=C/C(=O)N1CC[C@@H](Nc2n[nH]c3nccc(-c4ccc(C5(NC(=O)c6noc(C(C)(C)C)n6)CC5)cc4)c23)C1]

free-base; MH⁺=598.2.
(R)-5-(tert-butyl)-N-(1-(4-(3-((1-(3-methyloxetane-3-carbonyl)pyrrolidin-3-yl)amino)-1H-pyrazolo[3,4-b]pyridin-4-yl)phenyl)cyclopropyl)-1,2,4-oxadiazole-3-carboxamide (Compound-30)

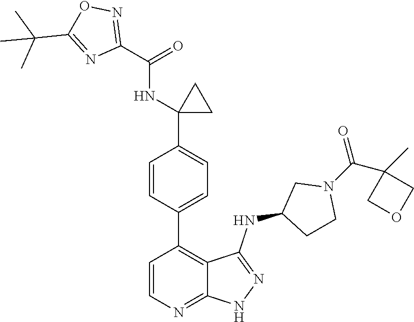 [SMILES: CC1(C(=O)N2CC[C@@H](Nc3n[nH]c4nccc(-c5ccc(C6(NC(=O)c7noc(C(C)(C)C)n7)CC6)cc5)c34)C2)COC1]

free-base; MH⁺=585.2.

### Example 5: Synthesis of 4-tert-butyl-N-{[2-fluoro-4-(3-{[(3R)-pyrrolidin-3-yl]amino}-1H-pyrazolo[3,4-b]pyridin-4-yl)phenyl]methyl}benzamide (Compound-8)

### 3.5 Preparation of (R)—N-(4-(3-((1-acryloylpyrrolidin-3-yl)amino)-1H-pyrazolo[3,4-b]pyridin-4-yl)-2-fluorobenzyl)-4-(tert-butyl)benzamide)

### 3.5.1 Preparation of tert-butyl (R)-3-((4-hydroxy-1-(4-methoxybenzyl)-1H-pyrazolo[3,4-b]pyridin-3-yl)amino)pyrrolidine-1-carboxylate (937A-2)

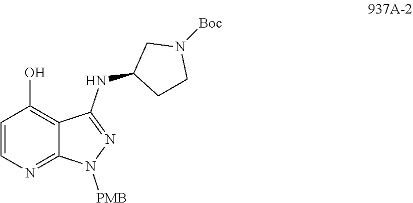

tert-Butyl (R)-3-((4-hydroxy-1-(4-methoxybenzyl)-1H-pyrazolo[3,4-b]pyridin-3-yl)amino)pyrrolidine-1-carboxylate (937A-2) (480 mg, 38%) was obtained as a yellow oil from 3-bromo-4-chloro-1-(4-methoxybenzyl)-1H-pyrazolo[3,4-b]pyridine (1.0 g, 2.85 mmol) and tert-butyl (R)-3-aminopyrrolidine-1-carboxylate (531 mg, 2.85 mmol) following a procedure adapted from Method 3.1.2. LC-MS (ESI): m/z 440.77.

### 3.5.2 Preparation of tert-butyl (R)-3-((1-(4-methoxybenzyl)-4-(((trifluoromethyl)sulfonyl)oxy)-1H-pyrazolo[3,4-b]pyridin-3-yl)amino)pyrrolidine-1-carboxylate (937A-3)

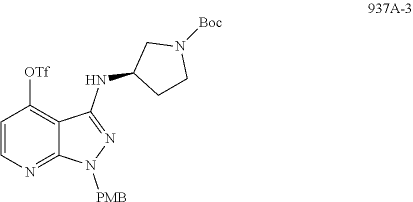

tert-Butyl (R)-3-((1-(4-methoxybenzyl)-4-(((trifluoromethyl)sulfonyl)oxy)-1H-pyrazolo[3,4-b]pyridin-3-yl)amino)pyrrolidine-1-carboxylate (937A-3) (2.6 g, quant.) was obtained as a yellow oil from tert-butyl (R)-3-((4-hydroxy-1-(4-methoxybenzyl)-1H-pyrazolo[3,4-b]pyridin-3-yl)amino)pyrrolidine-1-carboxylate (2.0 g, 4.55 mmol) following a procedure adapted from Method 3.1.3. LC-MS (ESI): m/z 572.68.

### 3.5.3 Preparation of tert-butyl (R)-3-((4-(4-((4-(tert-butyl)benzamido)methyl)-3-fluorophenyl)-1-(4-methoxybenzyl)-1H-pyrazolo[3,4-b]pyridin-3-yl)amino)pyrrolidine-1-carboxylate (937A-4)

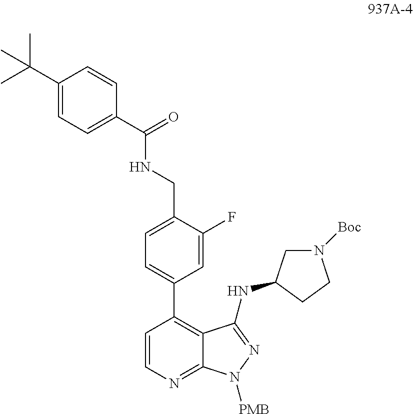

tert-Butyl (R)-3-((4-(4-((4-(tert-butyl)benzamido)methyl)-3-fluorophenyl)-1-(4-methoxybenzyl)-1H-pyrazolo[3,4-b]pyridin-3-yl)amino)pyrrolidine-1-carboxylate (937A-4) (100 mg, 67%) was obtained as a yellow oil from tert-butyl (R)-3-((1-(4-methoxybenzyl)-4-(((trifluoromethyl)sulfonyl)oxy)-1H-pyrazolo[3,4-b]pyridin-3-yl)amino)pyrrolidine-1-carboxylate (120 mg, 0.21 mmol) and 4-(tert-butyl)-N-(2-fluoro-4-(4,4,5,5-tetramethyl-1,3,2-dioxaborolan-2-yl)benzyl)benzamide (103 mg, 0.25 mmol) following a procedure adapted from Method 3.1.4. LC-MS (ESI): m/z (M+1) 707.86.

### 3.5.4 Preparation of (R)-4-(tert-butyl)-N-(2-fluoro-4-(1-(4-methoxybenzyl)-3-(pyrrolidin-3-ylamino)-1H-pyrazolo[3,4-b]pyridin-4-yl)benzyl)benzamide (937A-5)

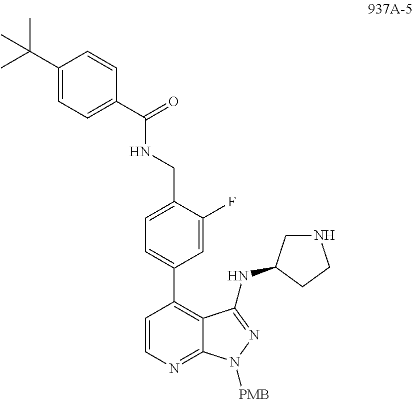

(R)-4-(tert-Butyl)-N-(2-fluoro-4-(1-(4-methoxybenzyl)-3-(pyrrolidin-3-ylamino)-1H-pyrazolo[3,4-b]pyridin-4-yl)benzyl)benzamide (937A-5) (214 mg, quant.) was obtained as a yellow solid by treating tert-butyl (R)-3-((4-(4-((4-(tert-butyl)benzamido)methyl)-3-fluorophenyl)-1-(4-methoxybenzyl)-1H-pyrazolo[3,4-b]pyridin-3-yl)amino)pyrrolidine-1-carboxylate (250 mg, 0.35 mmol) with TFA at RT. LC-MS (ESI): m/z (M+1) 607.79.

### 3.5.5 Preparation of (R)-4-(tert-butyl)-N-(2-fluoro-4-(3-(pyrrolidin-3-ylamino)-1H-pyrazolo[3,4-b]pyridin-4-yl)benzyl)benzamide (Compound-8)

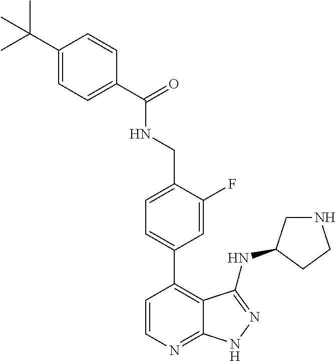 [SMILES: CC(C)(C)c1ccc(C(=O)NCc2ccc(-c3ccnc4[nH]nc(N[C@@H]5CCNC5)c34)cc2F)cc1]

(R)-4-(tert-butyl)-N-(2-fluoro-4-(3-(pyrrolidin-3-ylamino)-1H-pyrazolo[3,4-b]pyridin-4-yl)benzyl)benzamide was obtained as a yellow solid by treating tert-butyl (R)-3-((4-(4-((4-(tert-butyl)benzamido)methyl)-3-fluorophenyl)-1-(4-methoxybenzyl)-1H-pyrazolo[3,4-b]pyridin-3-yl)amino)pyrrolidine-1-carboxylate with TFA at 50° C. LC-MS (ESI): m/z (M+1) 487.66. ¹H NMR (400 MHz, DMSO) δ 12.56 (s, 1H), 9.11 (t, J=5.7 Hz, 1H), 8.43 (d, J=4.7 Hz, 1H), 8.36 (s, 1H), 7.87 (d, J=8.4 Hz, 2H), 7.59-7.39 (m, 5H), 6.99 (d, J=4.7 Hz, 1H), 4.60 (d, J=5.5 Hz, 2H), 4.53 (d, J=5.9 Hz, 1H), 4.11 (s, 1H), 3.16 (dd, J=11.4, 6.0 Hz, 1H), 3.07-2.89 (m, 3H), 2.06 (dt, J=20.3, 7.3 Hz, 1H), 1.68-1.58 (m, 1H), 1.31 (s, 9H).

### Example 6: Synthesis of 4-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}benzamide (Compound-6)

### 3.5.6 Preparation of (R)—N-(4-(3-((1-acryloylpyrrolidin-3-yl)amino)-1-(4-methoxybenzyl)-1H-pyrazolo[3,4-b]pyridin-4-yl)-2-fluorobenzyl)-4-(tert-butyl)benzamide (937A-6

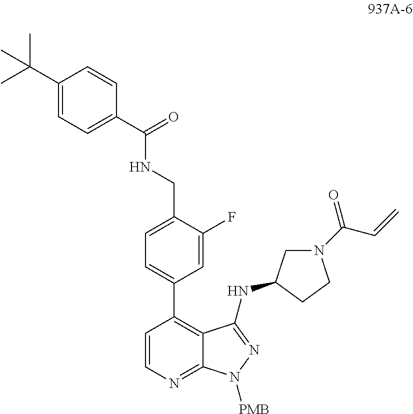

At 0° C., to a stirred solution of (R)-4-(tert-butyl)-N-(2-fluoro-4-(1-(4-methoxybenzyl)-3-(pyrrolidin-3-ylamino)-1H-pyrazolo[3,4-b]pyridin-4-yl)benzyl)benzamide (107 mg, 0.177 mmol) and DIPEA (0.15 mg, 0.89 mmol) in tetrahydrofuran (THF) (3 mL) was added acryloyl chloride (16 mg, 0.177 mmol, in 1 mL THF) dropwise. After being stirred at room temperature for 1 hr, the reaction mixture was quenched with sat. NaHCO₃. The layers were separated and the organic layer was washed with brine, dried over Na₂SO₄. Solvent was removed and the residue was purified by flash chromatography (silica gel, 0˜60 ethyl acetate in petroleum ether) to provide (R)—N-(4-(3-((1-acryloylpyrrolidin-3-yl)amino)-1-(4-methoxybenzyl)-1H-pyrazolo[3,4-b]pyridin-4-yl)-2-fluorobenzyl)-4-(tert-butyl)benzamide (937A-6) (quant.) as a yellow oil.

### 3.5.7 Preparation of (R)—N-(4-(3-((1-acryloylpyrrolidin-3-yl)amino)-1H-pyrazolo[3,4-b]pyridin-4-yl)-2-fluorobenzyl)-4-(tert-butyl)benzamide (937A)

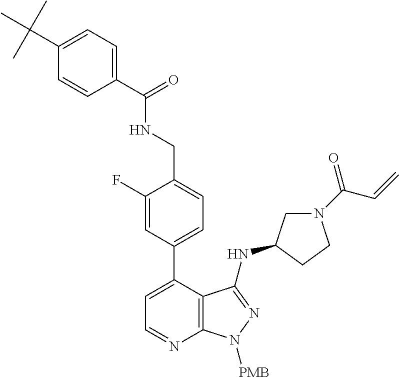

(R)—N-(4-(3-((1-Acryloylpyrrolidin-3-yl)amino)-1H-pyrazolo[3,4-b]pyridin-4-yl)-2-fluorobenzyl)-4-(tert-butyl)benzamide (937A) (38 mg, 40%) was obtained as a white solid from (R)—N-(4-(3-((1-acryloylpyrrolidin-3-yl)amino)-1-(4-methoxybenzyl)-1H-pyrazolo[3,4-b]pyridin-4-yl)-2-fluorobenzyl)-4-(tert-butyl)benzamide (0.177 mmol) following a procedure adapted from Method 3.1.5. LC-MS (ESI): m/z (M+1) 541.67. ¹H NMR (400 MHz, DMSO) δ 12.57 (s, 1H), 9.04 (d, J=6.6 Hz, 1H), 8.43 (d, J=4.5 Hz, 1H), 7.87 (dd, J=8.3, 4.3 Hz, 2H), 7.63-7.28 (m, 5H), 6.99 (d, J=4.1 Hz, 1H), 6.50 (dt, J=16.8, 9.7 Hz, 1H), 6.09 (dd, J=16.8, 2.2 Hz, 1H), 5.64-5.53 (m, 1H), 4.58 (d, J=5.3 Hz, 2H), 4.49 (dd, J=13.7, 5.4 Hz, 1H), 4.22-4.09 (m, 1H), 3.88-3.51 (m, 2H), 3.46-3.35 (m, 2H), 2.21-2.04 (m, 1H), 1.93-1.73 (m, 1H), 1.31 (s, 9H).

### Example 7: Synthesis of N-{[4-(3-{[(3R)-1-(but-2-ynoyl)pyrrolidin-3-yl]amino}-1H-pyrazolo[3,4-b]pyridin-4-yl)-2-fluorophenyl]methyl}-4-tert-butylbenzamide (Compound-7)

### 3.5.8 Preparation of (R)—N-(4-(3-((1-(but-2-ynoyl)pyrrolidin-3-yl)amino)-1H-pyrazolo[3,4-b]pyridin-4-yl)-2-fluorobenzyl)-4-(tert-butyl)benzamide (937B)

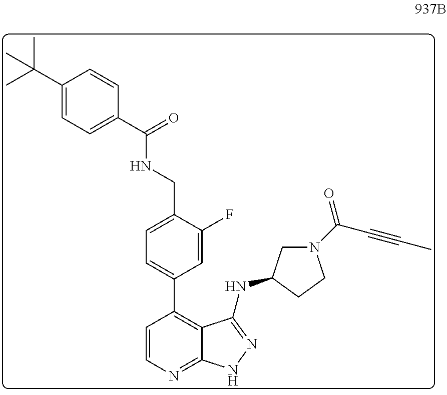 [SMILES: CC#CC(=O)N1CC[C@@H](Nc2n[nH]c3nccc(-c4ccc(CNC(=O)c5ccc(C(C)(C)C)cc5)c(F)c4)c23)C1]

(R)—N-(4-(3-((1-(But-2-ynoyl)pyrrolidin-3-yl)amino)-1H-pyrazolo[3,4-b]pyridin-4-yl)-2-fluorobenzyl)-4-(tert-butyl)benzamide (937B) (55 mg, 56%) was obtained as a white solid from (R)—N-(4-(3-((1-(but-2-ynoyl)pyrrolidin-3-yl)amino)-1-(4-methoxybenzyl)-1H-pyrazolo[3,4-b]pyridin-4-yl)-2-fluorobenzyl)-4-(tert-butyl)benzamide (107 mg, 0.177 mmol) following a procedure adapted from Method 3.1.5. LC-MS (ESI): m/z (M+1) 553.61. ¹H NMR (400 MHz, DMSO) δ 12.58 (d, J=3.5 Hz, 1H), 9.04 (t, J=5.2 Hz, 1H), 8.43 (dd, J=4.6, 2.7 Hz, 1H), 7.86 (d, J=8.3 Hz, 2H), 7.61-7.36 (m, 5H), 7.00 (dd, J=4.6, 2.5 Hz, 1H), 4.58 (dd, J=13.5, 4.9 Hz, 3H), 4.20-4.08 (m, 1H), 3.89-3.54 (m, 2H), 3.53-3.34 (m, 2H), 2.19-2.06 (m, 1H), 1.97 (d, J=10.4 Hz, 3H), 1.81 (td, J=13.6, 6.9 Hz, 1H), 1.31 (s, 9H).

### Example 8A: Synthesis of 4-tert-butyl-N-[2-methyl-3-(3-{[(3R)-1-(prop-2-enoyl)pyrrolidin-3-yl]amino}-1H-pyrazolo[3,4-b]pyridin-4-yl)phenyl]benzamide (Compound-9)

### Example 8B: Synthesis of N-[3-(3-{[(3R)-1-(but-2-ynoyl)pyrrolidin-3-yl]amino}-1H-pyrazolo[3,4-b]pyridin-4-yl)-2-methylphenyl]-4-tert-butylbenzamide (Compound-10)

### 3.6 Preparation of (R)—N-(3-(3-((1-acryloylpyrrolidin-3-yl)amino)-1H-pyrazolo[3,4-b]pyridin-4-yl)-2-methylphenyl)-4-(tert-butyl)benzamide (938A)

### 3.6.1 Preparation of tert-butyl (R)-3-((4-(3-(4-(tert-butyl)benzamido)-2-methylphenyl)-1-(4-methoxybenzyl)-1H-pyrazolo[3,4-b]pyridin-3-yl)amino)pyrrolidine-1-carboxylate (938A-1)

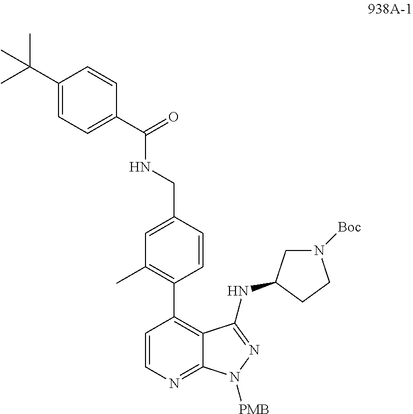

tert-Butyl (R)-3-((4-(3-(4-(tert-butyl)benzamido)-2-methylphenyl)-1-(4-methoxybenzyl)-1H-pyrazolo[3,4-b]pyridin-3-yl)amino)pyrrolidine-1-carboxylate (938A-1) (107 mg, 35%) was obtained as a yellow oil from tert-butyl (R)-3-((1-(4-methoxybenzyl)-4-(((trifluoromethyl)sulfonyl)oxy)-1H-pyrazolo[3,4-b]pyridin-3-yl)amino)pyrrolidine-1-carboxylate (250 mg, 0.44 mmol) and 4-(tert-butyl)-N-(2-methyl-3-(4,4,5,5-tetramethyl-1,3,2-dioxaborolan-2-yl)phenyl)benzamide (224 mg, 0.57 mmol) following a procedure adapted from Method 3.1.4. LC-MS (ESI): m/z (M+1) 689.87.

### 3.6.2 Preparation of (R)—N-(3-(3-((1-acryloylpyrrolidin-3-yl)amino)-1H-pyrazolo[3,4-b]pyridin-4-yl)-2-methylphenyl)-4-(tert-butyl)benzamide (938A) (Compound-9)

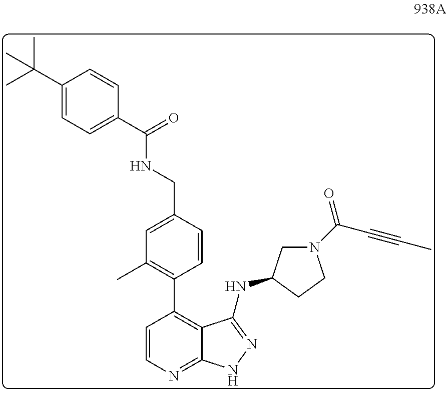 [SMILES: CC#CC(=O)N1CC[C@@H](Nc2n[nH]c3nccc(-c4ccc(CNC(=O)c5ccc(C(C)(C)C)cc5)cc4C)c23)C1]

(R)—N-(3-(3-((1-Acryloylpyrrolidin-3-yl)amino)-1H-pyrazolo[3,4-b]pyridin-4-yl)-2-methylphenyl)-4-(tert-butyl)benzamide (938A) (20 mg, 49%) was obtained as a white solid from tert-butyl (R)-3-((4-(3-(4-(tert-butyl)benzamido)-2-methylphenyl)-1-(4-methoxybenzyl)-1H-pyrazolo[3,4-b]pyridin-3-yl)amino)pyrrolidine-1-carboxylate (53 mg, 0.77 mmol) following a procedure adapted from Method 3.1.5. LC-MS (ESI): m/z (M+1) 523.69. ¹H NMR (400 MHz, DMSO) δ 12.52-12.40 (m, 1H), 10.16 (dd, J=32.9, 11.6 Hz, 1H), 8.46 (dd, J=4.6, 1.1 Hz, 1H), 8.00-7.89 (m, 2H), 7.55 (d, J=8.4 Hz, 2H), 7.47-7.34 (m, 2H), 7.24-7.11 (m, 1H), 6.91-6.81 (m, 1H), 6.57-6.41 (m, 1H), 6.15-5.99 (m, 1H), 5.64-5.40 (m, 1H), 4.27-4.09 (m, 1H), 3.91-3.66 (m, 2H), 3.63-3.42 (m, 2H), 2.21-1.98 (m, 1H), 1.96-1.85 (m, 3H), 1.83-1.70 (m, 1H), 1.32 (s, 9H).

### 3.7 Preparation of (R)—N-(3-(3-((1-(but-2-ynoyl)pyrrolidin-3-yl)amino)-1H-pyrazolo[3,4-b]pyridin-4-yl)-2-methylphenyl)-4-(tert-butyl)benzamide (938B)

### 3.7.1 Preparation of (R)—N-(3-(3-((1-(but-2-ynoyl)pyrrolidin-3-yl)amino)-1H-pyrazolo[3,4-b]pyridin-4-yl)-2-methylphenyl)-4-(tert-butyl)benzamide (938B) (Compound-10)

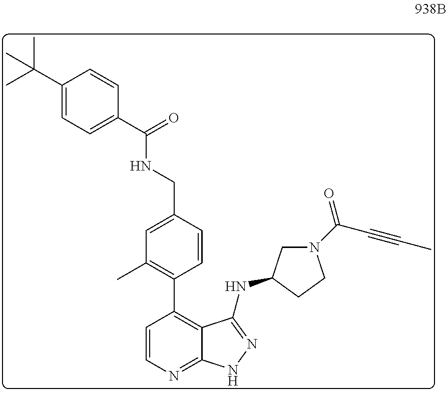 [SMILES: CC#CC(=O)N1CC[C@@H](Nc2n[nH]c3nccc(-c4ccc(CNC(=O)c5ccc(C(C)(C)C)cc5)cc4C)c23)C1]

(R)—N-(3-(3-((1-(But-2-ynoyl)pyrrolidin-3-yl)amino)-1H-pyrazolo[3,4-b]pyridin-4-yl)-2-methylphenyl)-4-(tert-butyl)benzamide (938B) (17 mg, 41%) was obtained as a white solid from tert-butyl (R)-3-((4-(3-(4-(tert-butyl)benzamido)-2-methylphenyl)-1-(4-methoxybenzyl)-1H-pyrazolo[3,4-b]pyridin-3-yl)amino)pyrrolidine-1-carboxylate (53 mg, 0.77 mmol) following a procedure adapted from Method 3.1.5. LC-MS (ESI): m/z (M+1) 535.70. ¹H NMR (400 MHz, DMSO) δ 12.47 (d, J=6.8 Hz, 1H), 10.20 (dd, J=21.2, 16.0 Hz, 1H), 8.47 (dd, J=4.5, 2.8 Hz, 1H), 7.95 (d, J=7.0 Hz, 2H), 7.55 (d, J=7.7 Hz, 2H), 7.49-7.33 (m, 2H), 7.28-7.12 (m, 1H), 6.87 (t, J=4.0 Hz, 1H), 4.22-4.11 (m, 1H), 3.95-3.74 (m, 2H), 3.64-3.43 (m, 3H), 2.24-1.72 (m, 7H), 1.40-1.16 (m, 9H).

### Example 9: Synthesis of (1r,4r)-4-({4-[3-fluoro-4-(hydroxymethyl)phenyl]-1H-pyrazolo[3,4-b]pyridin-3-yl})amino)cyclohexan-1-ol (Compound-11)

### 3.1. Preparation of (1r,4r)-4-((4-(3-fluoro-4-(hydroxymethyl)phenyl)-1H-pyrazolo[3,4-b]pyridin-3-yl)amino)cyclohexan-1-ol (934)

### 3.1.1. Preparation of (2-fluoro-4-(4,4,5,5-tetramethyl-1,3,2-dioxaborolan-2-yl)phenyl)methanol (934-2)

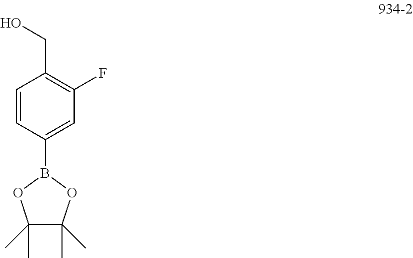 [SMILES: CC1(C)OB(c2ccc(CO)c(F)c2)OC1(C)C]

At 90° C. under N₂ atmosphere, to a stirred solution of 4-bromo-2-fluorophenylmethanol (500 mg, 2.44 mmol) in dioxane (5 mL) were added B₂Pin₂ (929 mg, 3.66 mmol), Pd(dppf)Cl₂.DCM (198 mg, 0.24 mmol) and KOAc (717 mg, 7.32 mmol). After being stirred at 90° C. overnight, the reaction mixture was cooled down to room temperature and partitioned between EA and H₂O. The layers were separated and the organic layer was washed with brine, dried over Na₂SO₄. Solvents were removed under vacuum and the residue was purified by flash chromatography (silica gel, 0˜20 ethyl acetate in petroleum ether) to provide (2-fluoro-4-(4,4,5,5-tetramethyl-1,3,2-dioxaborolan-2-yl)phenyl)methanol (934-2) (300 mg, 48%) as a yellow oil.

### 3.1.2. Preparation of 3-(((1r,4r)-4-((tert-butyldimethylsilyl)oxy)cyclohexyl)amino)-1-(4-methoxybenzyl)-1H-pyrazolo[3,4-b]pyridin-4-ol (934-4)

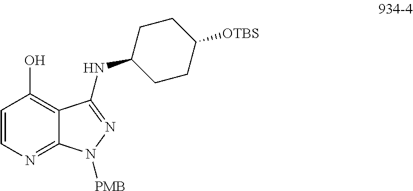

At 140° C. under N₂ atmosphere, to a stirred solution of 4-chloro-3-iodo-1-(4-methoxybenzyl)-1H-pyrazolo[3,4-b]pyridine (5.7 g, 14.3 mmol) in DMF (100 mL) were added (1r,4r)-4-((tert-butyldimethylsilyl)oxy)cyclohexan-1-amine (3.6 g, 15.7 mmol), Pd₂(dba)₃ (1.3 g, 1.43 mmol), Xantphos (1.65 g, 2.83 mmol) and Cs₂CO₃ (23.3 g, 71.5 mmol). After being stirred at 140° C. overnight, the reaction mixture was cooled down to room temperature and quenched with H₂O, extracted with DCM (3×). The combined organic layers were washed with brine, dried over Na₂SO₄. Solvents were removed and the residue was purified by flash chromatography (silica gel, 0˜20 ethyl acetate in petroleum ether) to provide 3-(((1r,4r)-4-((tert-butyldimethylsilyl)oxy)cyclohexyl)amino)-1-(4-methoxybenzyl)-1H-pyrazolo[3,4-b]pyridin-4-ol (934-4) (1.8 g, 26%) as a yellow oil. LC-MS (ESI): 483.63 m/z (M+1).

### 3.1.3. Preparation of 3-(((1r,4r)-4-((tert-butyldimethylsilyl)oxy)cyclohexyl)amino)-1-(4-methoxybenzyl)-1H-pyrazolo[3,4-b]pyridin-4-yl trifluoromethanesulfonate (934-5)

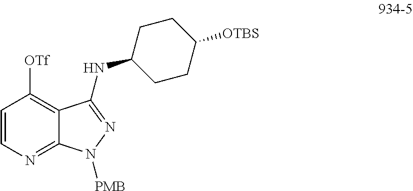

At 0° C. under N₂ atmosphere, to a stirred solution of (2-fluoro-4-(4,4,5,5-tetramethyl-1,3,2-dioxaborolan-2-yl)phenyl)methanol (1.89 g, 3.92 mmol) and TEA (2.2 mL, 15.2 mmol) in DCM (5 mL) was added Tf₂O (3.3 g, 11.7 mmol) dropwise. After being stirred at 0° C. for 1 hr, the reaction mixture was quenched with sat. NaHCO₃. The layers were separated and the aqueous layer was extracted with DCM (×2). The combined organic layers were washed with brine, dried over Na₂SO₄. Solvents were removed to provide the crude product 3-(((1r,4r)-4-((tert-butyldimethyl silyl)oxy)cyclohexyl)amino)-1-(4-methoxybenzyl)-1H-pyrazolo[3,4-b]pyridin-4-yl trifluoromethanesulfonate (934-5) (2.67 g, quant.) which was used in the next step without purification.

### 3.1.4. Preparation of (4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorophenyl)methanole (934-6)

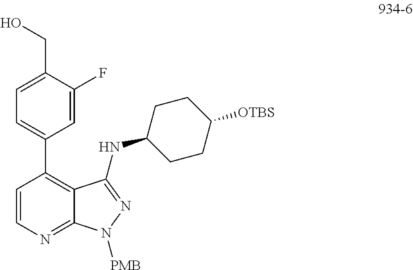

At 90° C. under N₂ atmosphere, to a stirred solution of 3-(((1r,4r)-4-((tert-butyldimethylsilyl)oxy)cyclohexyl)amino)-1-(4-methoxybenzyl)-1H-pyrazolo[3,4-b]pyridin-4-yl trifluoromethanesulfonate (70 mg, 0.114 mmol) and (2-fluoro-4-(4,4,5,5-tetramethyl-1,3,2-dioxaborolan-2-yl)phenyl)methanol (43 mg, 0.117 mmol) in dioxane/H₂O (2 mL/0.5 mL) were added Pd(PPh₃)₄ (13 mg, 0.01 mmol) and Na₂CO₃ (24 mg, 0.228 mmol). After being stirred at 90° C. overnight, the reaction mixture was cooled down to room temperature and partitioned between EA/H₂O. The layers were separated and the organic layer was dried over Na₂SO₄. Solvents were removed under vacuum and the residue was purified by flash chromatography (silica gel, 0˜20 ethyl acetate in petroleum ether) to provide (4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorophenyl)methanole (934-6) (35 mg, 52%) as a yellow oil. LC-MS (ESI): m/z (M+1) 591.77.

### 3.1.5. Preparation of (1r,4r)-4-((4-(3-fluoro-4-(hydroxymethyl)phenyl)-1H-pyrazolo[3,4-b]pyridin-3-yl)amino)cyclohexan-1-ol (934)

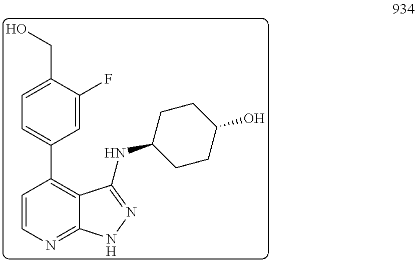 [SMILES: OCc1ccc(-c2ccnc3[nH]nc(N[C@H]4CC[C@H](O)CC4)c23)cc1F]

The solution of (4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorophenyl)methanole (35 mg, 0.06 mmol) in TFA (2 mL) was heated to 60° C. for 4 hr. After cooling down to room temperature, the reaction mixture was concentrated and 1N NaOH was added. The stirring was continued for another 2 hr and extracted with DCM (3×). The combined organic layers were washed with brine, and dried over Na₂SO₄. Solvents were removed under vacuum and the residue was purified by reverse phase HPLC (C18, 10%˜90% acetonitrile in H₂O with 0.1 formic acid) to provide (1r,4r)-4-((4-(3-fluoro-4-(hydroxymethyl)phenyl)-1H-pyrazolo[3,4-b]pyridin-3-yl)amino)cyclohexan-1-ol (934) (15 mg, 71%) as a white solid. LC-MS (ESI): m/z (M+1) 357.64. ¹H NMR (400 MHz, DMSO) δ 12.39 (s, 1H), 8.40 (d, J=4.7 Hz, 1H), 7.65 (t, J=7.9 Hz, 1H), 7.46-7.37 (m, 2H), 6.94 (d, J=4.7 Hz, 1H), 5.42 (s, 1H), 4.64 (s, 2H), 4.48 (s, 1H), 3.85 (d, J=7.2 Hz, 1H), 3.36 (s, 1H), 1.97 (d, J=9.9 Hz, 2H), 1.74 (d, J=9.4 Hz, 2H), 1.31-0.95 (m, 5H).

### Example 10: Synthesis of 4-tert-butyl-N-{[2-fluoro-4-(3-{[(1r,4r)-4-hydroxycyclohexyl]amino}-1H-pyrazolo[3,4-b]pyridin-4-yl)phenyl]methyl}benzamide (Compound-12)

### 3.2. Preparation of 4-(tert-butyl)-N-(2-fluoro-4-(3-(((1r,4r)-4-hydroxycyclohexyl)amino)-1H-pyrazolo[3,4-b]pyridin-4-yl)benzyl)benzamide (935)

### 3.2.1. Preparation of N-(4-bromo-2-fluorobenzyl)-4-(tert-butyl)benzamide (935-2)

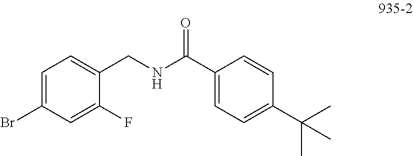 [SMILES: CC(C)(C)c1ccc(C(=O)NCc2ccc(Br)cc2F)cc1]

To a stirred solution of (4-bromo-2-fluorophenyl)methanamine (1.02 g, 5 mmol) and 4-(tert-butyl)benzoic acid (980 mg, 5.5 mmol) in DCM (10 mL) were added DIPEA (2.8 mL, 15 mmol) and (2-(1H-benzotriazol-1-yl)-1,1,3,3-tetramethyluronium hexafluorophosphate) (HBTU) (2.84 g, 7.5 mmol). After being stirred at room temperature overnight, the reaction mixture was cooled down to room temperature and quenched with saturated NaHCO₃. The layers were separated and the organic layer was washed with brine, dried over Na₂SO₄. Solvent was removed and the residue was purified by flash chromatography (silica gel, 0˜20 ethyl acetate in petroleum ether) to provide N-(4-bromo-2-fluorobenzyl)-4-(tert-butyl)benzamide (935-2) (1.6 g, 88%) as a yellow oil. LC-MS (ESI): m/z (M/M+2) 364.43/366.44.

### 3.2.2. Preparation of 4-(tert-butyl)-N-(2-fluoro-4-(4,4,5,5-tetramethyl-1,3,2-dioxaborolan-2-yl)benzyl)benzamide (935-3)

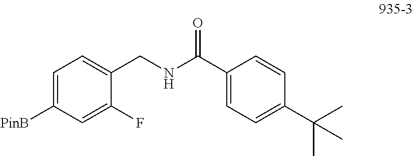

4-(tert-Butyl)-N-(2-fluoro-4-(4,4,5,5-tetramethyl-1,3,2-dioxaborolan-2-yl)benzyl)benzamide (935-3) (1.5 g, 80%) was obtained as a yellow oil from N-(4-bromo-2-fluorobenzyl)-4-(tert-butyl)benzamide (1.6 g, 4.57 mmol) following a procedure adapted from Method 3.1.1. LC-MS (ESI): m/z (M+1) 412.60.

### 3.2.3. Preparation of 4-(tert-butyl)-N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorobenzyl)benzamide (935-4)

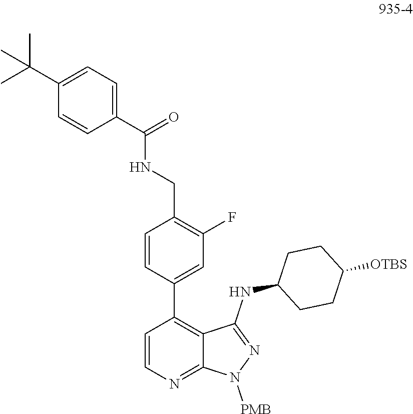

4-(tert-Butyl)-N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorobenzyl)benzamide (935-4) (55 mg, 56%) was obtained as a yellow oil from 3-(((1r,4r)-4-((tert-butyldimethylsilyl)oxy)cyclohexyl)amino)-1-(4-methoxybenzyl)-1H-pyrazolo[3,4-b]pyridin-4-yl trifluoromethanesulfonate (80 mg, 0.13 mmol) and 4-(tert-butyl)-N-(2-fluoro-4-(4,4,5,5-tetramethyl-1,3,2-dioxaborolan-2-yl)benzyl)benzamide (80 mg, 0.19 mmol) following a procedure adapted from Method 3.1.4. LC-MS (ESI): m/z (M−1+46) 795.37.

### 3.2.4. Preparation of 4-(tert-butyl)-N-(2-fluoro-4-(3-(((1r,4r)-4-hydroxycyclohexyl)amino)-1H-pyrazolo[3,4-b]pyridin-4-yl)benzyl)benzamide (935)

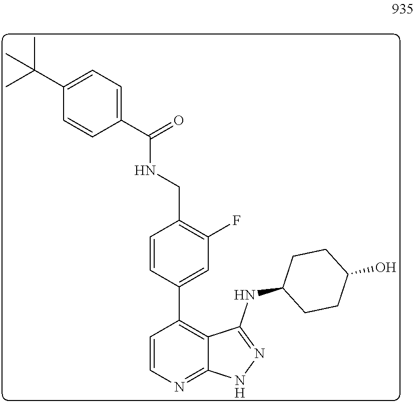 [SMILES: CC(C)(C)c1ccc(C(=O)NCc2ccc(-c3ccnc4[nH]nc(N[C@H]5CC[C@H](O)CC5)c34)cc2F)cc1]

4-(tert-Butyl)-N-(2-fluoro-4-(3-(((1r,4r)-4-hydroxycyclohexyl)amino)-1H-pyrazolo[3,4-b]pyridin-4-yl)benzyl)benzamide (935) (24 mg, 64%) was obtained as a white solid from 4-(tert-butyl)-N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorobenzyl)benzamide (55 mg, 0.073 mmol) following a procedure adapted from Method 3.1.5. LC-MS (ESI): m/z (M+1) 516.78.

¹H NMR (400 MHz, DMSO) δ 12.39 (s, 1H), 9.05 (t, J=5.9 Hz, 1H), 8.40 (d, J=4.7 Hz, 1H), 7.85 (d, J=8.5 Hz, 2H), 7.59-7.35 (m, 5H), 6.93 (d, J=4.7 Hz, 1H), 4.59 (d, J=5.7 Hz, 2H), 4.46 (d, J=3.7 Hz, 1H), 3.81 (d, J=7.2 Hz, 1H), 3.35 (s, 2H), 1.95 (d, J=10.1 Hz, 2H), 1.72 (d, J=9.7 Hz, 2H), 1.31 (s, 9H), 1.17 (dd, J=22.5, 9.5 Hz, 2H), 1.04 (dd, J=22.7, 10.0 Hz, 2H).

### Example 11: Synthesis of 4-tert-butyl-N-[3-(3-{[(1r,4r)-4-hydroxycyclohexyl]amino}-1H-pyrazolo[3,4-b]pyridin-4-yl)phenyl]benzamide (Compound-13)

### 3.2.5. Preparation of 4-(tert-butyl)-N-(3-(3-(((1r,4r)-4-hydroxycyclohexyl)amino)-1H-pyrazolo[3,4-b]pyridin-4-yl)phenyl)benzamide (936A)

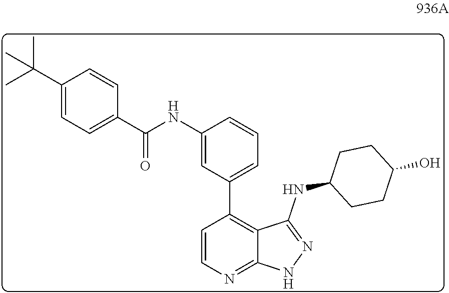 [SMILES: CC(C)(C)c1ccc(C(=O)Nc2cccc(-c3ccnc4[nH]nc(N[C@H]5CC[C@H](O)CC5)c34)c2)cc1]

4-(tert-Butyl)-N-(3-(3-(((1r,4r)-4-hydroxycyclohexyl)amino)-1H-pyrazolo[3,4-b]pyridin-4-yl)phenyl)benzamide (936A) (20 mg, 69%) was obtained as a white solid from 4-(tert-butyl)-N-(3-(3-(((1r,4r)-4-((tert-butyldimethylsilyl)oxy)cyclohexyl)amino)-1-(4-methoxybenzyl)-1H-pyrazolo[3,4-b]pyridin-4-yl)phenyl)benzamide (42 mg, 0.06 mmol) following a procedure adapted from Method 3.1.5. LC-MS (ESI): m/z (M+1) 484.73. ¹H NMR (400 MHz, DMSO) δ 12.34 (s, 1H), 10.41 (s, 1H), 8.41 (d, J=4.7 Hz, 1H), 8.08 (s, 1H), 7.91 (d, J=8.4 Hz, 2H), 7.83 (d, J=8.0 Hz, 1H), 7.56 (t, J=9.0 Hz, 3H), 7.29 (d, J=7.5 Hz, 1H), 6.92 (d, J=4.7 Hz, 1H), 4.46 (s, 1H), 4.05 (d, J=7.4 Hz, 1H), 3.36 (s, 2H), 1.99 (d, J=10.8 Hz, 2H), 1.71 (d, J=10.4 Hz, 2H), 1.33 (s, 9H), 1.15 (dt, J=30.6, 11.5 Hz, 4H).

### Example 12: Synthesis of 4-tert-butyl-N-[3-(3-{[(1r,4r)-4-hydroxycyclohexyl]amino}-1H-pyrazolo[3,4-b]pyridin-4-yl)phenyl]benzamide (Compound-14)

### 3.3. Preparation of 4-(tert-butyl)-N-(3-(3-(((1r,4r)-4-hydroxycyclohexyl)amino)-1H-pyrazolo[3,4-b]pyridin-4-yl)-2-methylphenyl)benzamide (936B)

### 3.3.1. Preparation of N-(3-bromo-2-methylphenyl)-4-(tert-butyl)benzamide (936B-2)

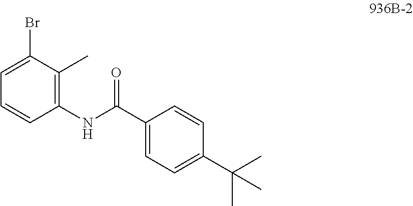 [SMILES: Cc1c(Br)cccc1NC(=O)c1ccc(C(C)(C)C)cc1]

N-(3-Bromo-2-methylphenyl)-4-(tert-butyl)benzamide (936B-2) (3.5 g, 100%) was obtained as a white solid from 3-bromo-2-methylaniline (1.9 g, 10 mmol) and 4-(tert-butyl)benzoic acid (1.96 g, 11 mmol) following a procedure adapted from Method 3.2.1. LC-MS (ESI): m/z (M/M+2) 350.39/352.40

### 3.3.2. Preparation of 4-(tert-butyl)-N-(2-methyl-3-(4,4,5,5-tetramethyl-1,3,2-dioxaborolan-2-yl)phenyl)benzamide (936B-3)

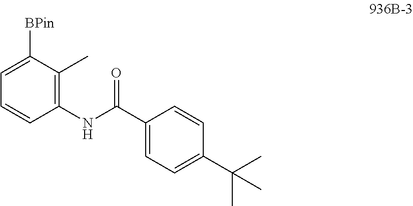

4-(tert-Butyl)-N-(2-methyl-3-(4,4,5,5-tetramethyl-1,3,2-dioxaborolan-2-yl)phenyl)benzamide (936B-3) (3.54 g, 89%) was obtained as a yellow solid from N-(3-bromo-2-methylphenyl)-4-(tert-butyl)benzamide (3.5 g, 7.2 mmol) following a procedure adapted from Method 3.1.1. LC-MS (ESI): m/z (M+1) 394.61.

### 3.3.3. Preparation of 4-(tert-butyl)-N-(3-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-methylphenyl)benzamide (936B-4)

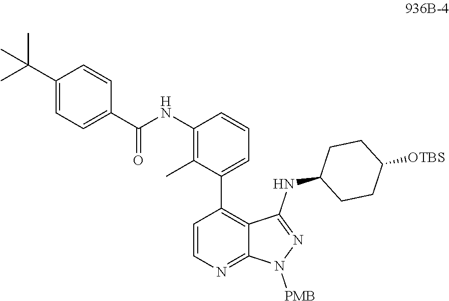

4-(tert-Butyl)-N-(3-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-methylphenyl)benzamide (936B-4) (68 mg, 71%) was obtained as a yellow oil from 3-(((1r,4r)-4-((tert-butyldimethylsilyl)oxy)cyclohexyl)amino)-1-(4-methoxybenzyl)-1H-pyrazolo[3,4-b]pyridin-4-yl trifluoromethanesulfonate (80 mg, 0.13 mmol) and 4-(tert-butyl)-N-(2-methyl-3-(4,4,5,5-tetramethyl-1,3,2-dioxaborolan-2-yl)phenyl)benzamide (75 mg, 0.19 mmol) following a procedure adapted from Method 3.1.4. LC-MS (ESI): m/z (M−1) 731.06.

### 3.3.4. Preparation of 4-(tert-butyl)-N-(3-(3-(((1r,4r)-4-hydroxycyclohexyl)amino)-1H-pyrazolo[3,4-b]pyridin-4-yl)-2-methylphenyl)benzamide (936B)

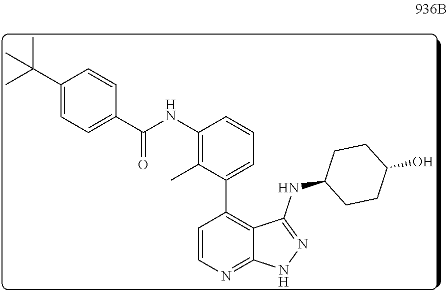 [SMILES: Cc1c(NC(=O)c2ccc(C(C)(C)C)cc2)cccc1-c1ccnc2[nH]nc(N[C@H]3CC[C@H](O)CC3)c12]

4-(tert-Butyl)-N-(3-(3-(((1r,4r)-4-hydroxycyclohexyl)amino)-1H-pyrazolo[3,4-b]pyridin-4-yl)-2-methylphenyl)benzamide (936B) (18 mg, 43%) was obtained as a white solid from 4-(tert-butyl)-N-(3-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-methylphenyl)benzamide (68 mg, 0.093 mmol) following a procedure adapted from Method 3.1.5. LC-MS (ESI): m/z (M+1) 498.80. ¹H NMR (400 MHz, DMSO) δ 12.26 (s, 1H), 10.16 (s, 1H), 8.42 (d, J=4.7 Hz, 1H), 7.94 (d, J=8.5 Hz, 2H), 7.56 (d, J=8.5 Hz, 2H), 7.47-7.37 (m, 2H), 7.18 (d, J=7.3 Hz, 1H), 6.80 (d, J=4.7 Hz, 1H), 4.47 (s, 1H), 3.43 (d, J=8.0 Hz, 2H), 2.00 (d, J=13.5 Hz, 1H), 1.93 (s, 3H), 1.72 (d, J=6.7 Hz, 3H), 1.33 (s, 9H), 1.24-1.09 (m, 4H).

### Example 13: Synthesis of 4-tert-butyl-N-[2-fluoro-4-(3-{[(1r,4r)-4-hydroxycyclohexyl]amino}-1H-pyrazolo[3,4-b]pyridin-4-yl)phenyl]benzamide (Compound-15)

### Preparation of 4-(tert-butyl)-N-(2-fluoro-4-(3-(((1r,4r)-4-hydroxycyclohexyl)amino)-1H-pyrazolo[3,4-b]pyridin-4-yl)phenyl)benzamide (939)

### 3.7.2 Preparation of N-(4-bromo-2-fluorophenyl)-4-(tert-butyl)benzamide (939-2)

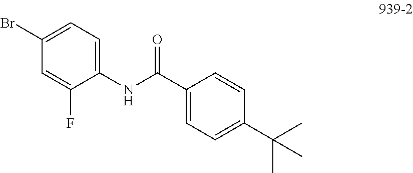 [SMILES: CC(C)(C)c1ccc(C(=O)Nc2ccc(Br)cc2F)cc1]

N-(4-Bromo-2-fluorophenyl)-4-(tert-butyl)benzamide (939-2) (3.5 g, 100%) was obtained as a white solid from 4-bromo-2-fluoroaniline (1.9 g, 10 mmol) and 4-(tert-butyl)benzoic acid (1.96 g, 11 mmol) following a procedure adapted from Method 3.2.1. LC-MS (ESI): m/z (M/M+2) 350.39/352.40.

### 3.7.3 Preparation of 4-(tert-butyl)-N-(2-fluoro-4-(4,4,5,5-tetramethyl-1,3,2-dioxaborolan-2-yl)phenyl)benzamide (939-3)

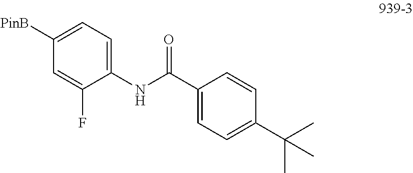

4-(tert-Butyl)-N-(2-fluoro-4-(4,4,5,5-tetramethyl-1,3,2-dioxaborolan-2-yl)phenyl)benzamide (939-3) (2.16 g, 75%) was obtained as a yellow solid from N-(4-bromo-2-fluorophenyl)-4-(tert-butyl)benzamide (2.53 g, 7.2 mmol) following a procedure adapted from Method 3.1.1. LC-MS (ESI): m/z 398.57

### 3.7.4 Preparation of 4-(tert-butyl)-N-(4-(3-(((1r,4r)-4-((tert-butyldimethylsilyl) oxy)cyclohexyl)amino)-1-(4-methoxybenzyl)-1H-pyrazolo[3,4-b]pyridin-4-yl)-2-fluorophenyl)benzamide (939-4)

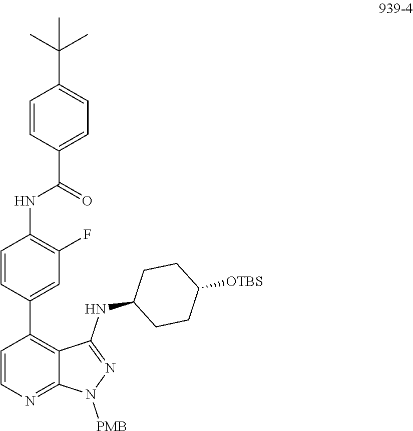

4-(tert-Butyl)-N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorophenyl)benzamide (939-4) (45 mg, 53%) was obtained as a yellow oil from 3-(((1r,4r)-4-((tert-butyldimethylsilyl)oxy)cyclohexyl)amino)-1-(4-methoxybenzyl)-1H-pyrazolo[3,4-b]pyridin-4-yl trifluoromethanesulfonate (70 mg, 0.11 mmol) and 4-(tert-butyl)-N-(2-fluoro-4-(4,4,5,5-tetramethyl-1,3,2-dioxaborolan-2-yl)phenyl)benzamide (80 mg, 0.17 mmol) following a procedure adapted from Method 3.1.4. LC-MS (ESI): m/z (M+1) 736.85.

### 3.7.5 Preparation of 4-(tert-butyl)-N-(2-fluoro-4-(3-(((1r,4r)-4-hydroxycyclohexyl)amino)-1H-pyrazolo[3,4-b]pyridin-4-yl)phenyl)benzamide (939)

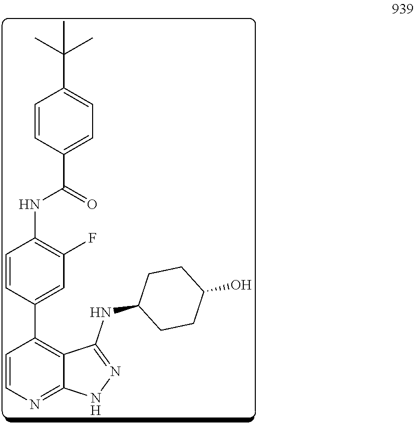 [SMILES: CC(C)(C)c1ccc(C(=O)Nc2ccc(-c3ccnc4[nH]nc(N[C@H]5CC[C@H](O)CC5)c34)cc2F)cc1]

4-(tert-Butyl)-N-(2-fluoro-4-(3-(((1r,4r)-4-hydroxycyclohexyl)amino)-1H-pyrazolo[3,4-b]pyridin-4-yl)phenyl)benzamide (939) (16 mg, 52%) was obtained as a white solid from 4-(tert-butyl)-N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorophenyl)benzamide (45 mg, 0.061 mmol) following a procedure adapted from Method 3.1.5. LC-MS (ESI): m/z (M+1) 502.72.

### Example 14: Synthesis of [2-(6-cyclopropyl-8-fluoro-1-oxo-1,2-dihydroisoquinolin-2-yl)-6-(3-{[(3R)-1-(prop-2-enoyl)pyrrolidin-3-yl]amino}-1H-pyrazolo[3,4-b]pyridin-4-yl)phenyl]methyl acetate (Compound-16)

### Preparation of tert-butyl (R)-3-((4-(2-(acetoxymethyl)-3-(6-cyclopropyl-8-fluoro-1-oxoisoquinolin-2(H)-yl)phenyl)-1-(4-methoxybenzyl)-1H-pyrazolo[3,4-b]pyridin-3-yl)amino)pyrrolidine-1-carboxylate

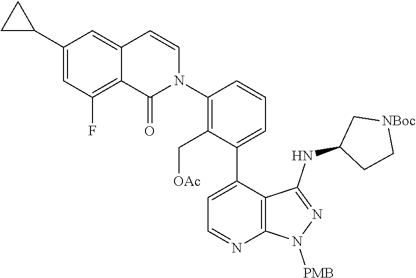

To a stirred solution of (R)-3-((1-(4-methoxybenzyl)-4-(((trifluoromethyl)sulfonyl)oxy)-1H-pyrazolo[3,4-b]pyridin-3-yl)amino)pyrrolidine-1-carboxylate (136.8 mg, 0.239 mmol) in 1,4-dioxane (2.1 mL) and water (0.25 mL) under nitrogen atmosphere were added 2-(6-cyclopropyl-8-fluoro-1-oxoisoquinolin-2(1H)-yl)-6-(4,4,5,5-tetramethyl-1,3,2-dioxaborolan-2-yl)benzyl acetate (190.2 mg, 0.398 mmol), Pd(OAc)₂ (10.8 mg, 0.048 mmol), Sphos (39.7 mg, 0.097 mmol) and K₂CO₃ (99.6 mg, 0.72 mmol). After stirring at 100° C. for one hour, the reaction mixture was cooled down to room temperature and partitioned between EtOAc and water. The combined organic layers were washed with brine, then dried over Na₂SO₄. Solvents were removed and the residue was purified by flash chromatography (silica gel, 30%˜50% ethyl acetate in hexanes) to provide the title compound as an orange oil (134.8 mg, 0.174 mmol, 73% yield). LC-MS (ESI): m/z (M+1): 773.4.

### Preparation of (R)-2-(6-cyclopropyl-8-fluoro-1-oxoisoquinolin-2(1H)-yl)-6-(3-(pyrrolidin-3-ylamino)-1H-pyrazolo[3,4-b]pyridin-4-yl)benzyl acetate

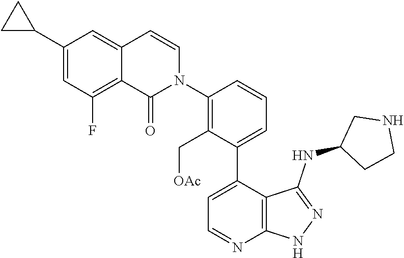 [SMILES: CC(=O)OCc1c(-c2ccnc3[nH]nc(N[C@@H]4CCNC4)c23)cccc1-n1ccc2cc(C3CC3)cc(F)c2c1=O]

A stirred solution of tert-butyl (R)-3-((4-(2-(acetoxymethyl)-3-(6-cyclopropyl-8-fluoro-1-oxoisoquinolin-2(1H)-yl)phenyl)-1-(4-methoxybenzyl)-1H-pyrazolo[3,4-b]pyridin-3-yl)amino)pyrrolidine-1-carboxylate (134.8 mg, 0.174 mmol) in TFA (5.8 mL) was heated to 50° C. overnight. The reaction mixture was cooled to room temperature, poured into water, and extracted with EtOAc. The combined organic layers were washed with 1N NaOH (aqueous solution) and brine, then dried over Na₂SO₄. Solvents were removed and the crude residue was carried forward without further purification. LC-MS (ESI): m/z (M+1): 553.2.

### Preparation of (R)-2-(3-((1-acryloylpyrrolidin-3-yl)amino)-1H-pyrazolo[3,4-b]pyridin-4-yl)-6-(6-cyclopropyl-8-fluoro-1-oxoisoquinolin-2(1H)-yl)benzyl acetate

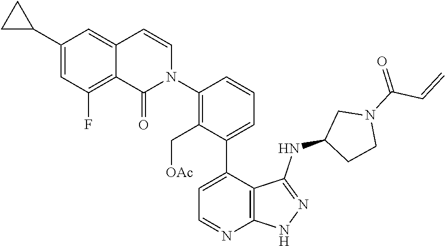 [SMILES: C=CC(=O)N1CC[C@@H](Nc2n[nH]c3nccc(-c4cccc(-n5ccc6cc(C7CC7)cc(F)c6c5=O)c4COC(C)=O)c23)C1]

To a stirred solution of (R)-2-(6-cyclopropyl-8-fluoro-1-oxoisoquinolin-2(1H)-yl)-6-(3-(pyrrolidin-3-ylamino)-1H-pyrazolo[3,4-b]pyridin-4-yl)benzyl acetate (11.5 mg, 0.0225 mmol) and DIPEA (12 μL, 0.069 mmol) in DCM (2.1 mL) under nitrogen atmosphere at −78° C. was added acryloyl chloride (1.8 μL, 0.022 mmol) in 1 mL DCM dropwise. After stirring for 5 mins, the reaction was quenched with several drops of formic acid. The reaction mixture was warmed to room temperature and the solvents were removed under vacuum. The residue was purified by reverse phase HPLC (C18, 20%˜50% acetonitrile in H₂O with 0.1% formic acid) to furnish the title compound as an orange solid (10.7 mg, 0.0176 mmol, 78% yield). LC-MS (ESI): m/z (M+1): 607.2.

### Example 15: Synthesis of 6-cyclopropyl-8-fluoro-2-[2-(hydroxymethyl)-3-(3-{[(3R)-1-(prop-2-enoyl)pyrrolidin-3-yl]amino}-1H-pyrazolo[3,4-b]pyridin-4-yl)phenyl]-1,2-dihydroisoquinolin-1-one (Compound-18) and 6-cyclopropyl-8-fluoro-2-[2-(hydroxymethyl)-3-(3-{[(3R)-1-(prop-2-enoyl)pyrrolidin-3-yl]amino}-1H-pyrazolo[3,4-b]pyridin-4-yl)phenyl]-1,2-dihydroisoquinolin-1-one (Compound-17)

### Preparation of (R)-6-cyclopropyl-8-fluoro-2-(2-(hydroxy methyl)-3-(3-(pyrrolidin-3-ylamino)-1H-pyrazolo[3,4-b]pyridin-4-yl)phenyl)isoquinolin-1 (2H)-one (Compound-17)

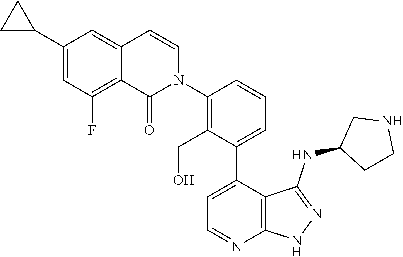 [SMILES: O=c1c2c(F)cc(C3CC3)cc2ccn1-c1cccc(-c2ccnc3[nH]nc(N[C@@H]4CCNC4)c23)c1CO]

To a stirred solution of (R)-2-(6-cyclopropyl-8-fluoro-1-oxoisoquinolin-2(1H)-yl)-6-(3-(pyrrolidin-3-ylamino)-1H-pyrazolo[3,4-b]pyridin-4-yl)benzyl acetate (70.9 mg, 0.128 mmol) in isopropyl alcohol (2.0 mL) and THF (2.0 mL) was added LiOH.H₂O (22.6 mg, 0.539 mmol). The reaction mixture was heated to 50° C. for 5 hours. The solvents were removed under vacuum and the crude residue was redissolved in THF. The organic layer was washed with brine then dried over Na₂SO₄. The solvents were removed under vacuum to afford the title compound as a yellow solid (42.3 mg, 0.083 mmol, 65% yield). A portion of this was purified by reverse phase HPLC (C18, 20%˜50% acetonitrile in H₂O with 0.1% formic acid). (ESI): m/z (M+1): 511.2.

### Preparation of (R)-2-(3-(3-((1-acryloylpyrrolidin-3-yl)amino)-1H-pyrazolo[3,4-b]pyridin-4-yl)-2-(hydroxymethyl)phenyl)-6-cyclopropyl-8-fluoroisoquinolin-1 (2H)-one (Compound-18)

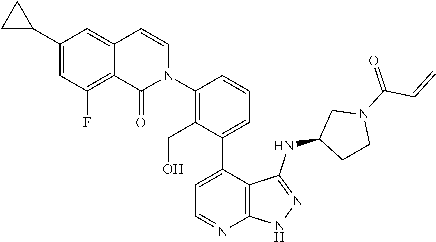 [SMILES: C=CC(=O)N1CC[C@@H](Nc2n[nH]c3nccc(-c4cccc(-n5ccc6cc(C7CC7)cc(F)c6c5=O)c4CO)c23)C1]

To a stirred solution of (R)-6-cyclopropyl-8-fluoro-2-(2-(hydroxymethyl)-3-(3-(pyrrolidin-3-ylamino)-1H-pyrazolo[3,4-b]pyridin-4-yl)phenyl)isoquinolin-1(2H)-one (10.7 mg, 0.0210 mmol) and DIPEA (11 μL, 0.063 mmol) in DCM (2.0 mL) under nitrogen atmosphere at −78° C. was added acryloyl chloride (1.7 μL, 0.021 mmol) in 1 mL DCM dropwise. After stirring for 5 mins, the reaction was quenched with several drops of formic acid. The reaction mixture was warmed to room temperature and the solvents were removed under vacuum. The residue was purified by reverse phase HPLC (C18, 20%˜50% acetonitrile in H₂O with 0.1% formic acid) to furnish the title compound as a yellow solid (1.7 mg, 0.0030 mmol, 14% yield). LC-MS (ESI): m/z (M+1): 565.2.

### Example 16: Synthesis of N-{[2-fluoro-4-(3-{[(1r,4r)-4-hydroxycyclohexyl]amino}-1H-pyrazolo[3,4-b]pyridin-4-yl)phenyl]methyl}-4,4-dimethylpentanamide (Compound-19)

### 3.8 Preparation of N-(2-fluoro-4-(3-(((1r,4r)-4-hydroxycyclohexyl)amino)-1H-pyrazolo[3,4-b]pyridin-4-yl)benzyl)-4,4-dimethylpentanamide (944)

### 3.8.1 Preparation of (1r,4r)-4-((4-(4-(aminomethyl)-3-fluorophenyl)-1-(4-methoxybenzyl)-1H-pyrazolo[3,4-b]pyridin-3-yl)amino)cyclohexan-1-ol (944-1)

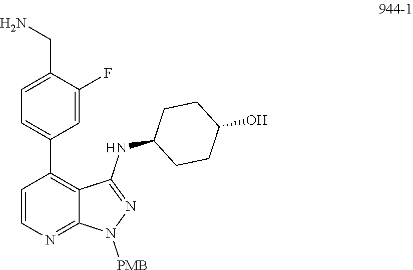

(1r,4r)-4-((4-(4-(Aminomethyl)-3-fluorophenyl)-1-(4-methoxybenzyl)-1H-pyrazolo[3,4-b]pyridin-3-yl)amino)cyclohexan-1-ol (944-1) (290 mg, 90%) was obtained as a yellow solid from tert-butyl (4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorobenzyl)carbamate (466 mg, 0.68 mmol) following a similar procedure outlined in Method 3.5.4. LC-MS (ESI): m/z (M+1) 476.56.

### 3.8.2 Preparation of 4-(4-(aminomethyl)-3-fluorophenyl)-N-((1r,4r)-4-((tert-butyldimethylsilyl)oxy)cyclohexyl)-1-(4-methoxybenzyl)-1H-pyrazolo[3,4-b]pyridin-3-amine (944-2)

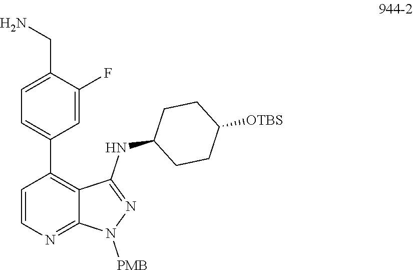

At 0° C., to a stirred solution of (1r,4r)-4-((4-(4-(aminomethyl)-3-fluorophenyl)-1-(4-methoxybenzyl)-1H-pyrazolo[3,4-b]pyridin-3-yl)amino)cyclohexan-1-ol (290 mg, 0.61 mmol) and imidazole (107 mg, 1.2 mmol) in DCM (5 mL) was added tert-butyldimethylsilyl chloride (TBSCl) (138 mg, 0.92 mmol). After being stirred at room temperature for 4 hr, the reaction mixture was quenched with H₂O. The layers were separated and the organic layer was dried. Solvent was removed under vacuum and the residue was purified by flash chromatography (silica gel, 0˜60 ethyl acetate in petroleum ether) to provide 4-(4-(aminomethyl)-3-fluorophenyl)-N-((1r,4r)-4-((tert-butyldimethyl silyl)oxy)cyclohexyl)-1-(4-methoxybenzyl)-1H-pyrazolo[3,4-b]pyridin-3-amine (944-2) (quant.) as a white solid. LC-MS (ESI): m/z (M+1) 590.67.

### 3.8.3 Preparation of ethyl (E)-4,4-dimethylpent-2-enoate (944-4)

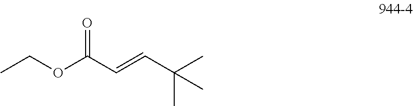

At 0° C., to a stirred solution of pivalaldehyde (2.0 g, 23 mmol) in THF (20 mL) was added NaH (930 mg, 23 mmol). After being stirred at room temperature for 0.5 hr, ethyl 2-(diethoxyphosphoryl)acetate (5.15 g, 23 mmol) was added dropwise, and the resulting solution was stirred at room temperature for another 4 hr. Then the reaction mixture was quenched with water and extracted with EtOAc. The organic layer was collected and dried. Solvent was removed under vacuum and the crude ethyl (E)-4,4-dimethylpent-2-enoate (944-4) (3.0 g, 83%) as a yellow oil without further purification. LC-MS (ESI): m/z (M+1) 157.12.

### 3.8.4 Preparation of ethyl 4,4-dimethylpentanoate (944-5)

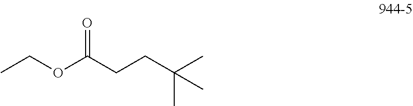

At room temperature, to a stirred solution of ethyl (E)-4,4-dimethylpent-2-enoate (1.5 g, 9.6 mmol) in MeOH (15 mL) was added Pd/C (150 mg, 10% wt). Under an atmosphere of hydrogen, the mixture was stirred at room temperature for 4 hr. Then the suspension was filtered and the filtrate evaporated to give the crude ethyl 4,4-dimethylpentanoate (944-5) (1.4 g, 93%) as a yellow oil without further purification. LC-MS (ESI): m/z (M+1) 158.13.

### 3.8.5 Preparation of 4,4-dimethylpentanoic acid (944-6)

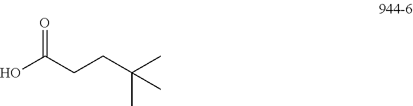

At room temperature, to a stirred solution of ethyl 4,4-dimethylpentanoate (1.4 g, 8.8 mmol) in a mixture of EtOH (10 ml) and water (3 mL) was added NaOH (800 mg, 20 mmol). The mixture was stirred at 80° C. for 6 hr. The resulting solution was acidified to pH 1 by addition of 6 N HCl solution and then the mixture was extracted with EA (20 mL×3). The combined organic layers were washed with brine, dried over Na₂SO₄, filtered to provide the crude 4,4-dimethylpentanoic acid (944-6) (800 mg, 70%) as a yellow oil without further purification. LC-MS (ESI): m/z (M+1) 131.10.

### 3.8.6 Preparation of N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorobenzyl)-4, 4-dimethylpentanamide (944-7)

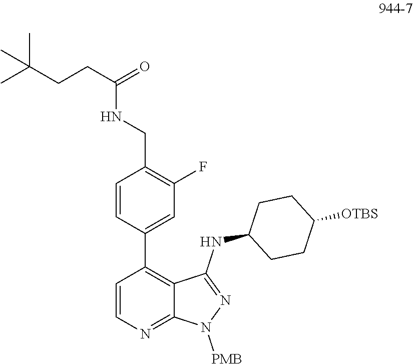

N-(4-(3-(((1r,4r)-4-((tert-Butyldimethylsilyl)oxy)cyclohexyl)amino)-1-(4-methoxybenzyl)-1H-pyrazolo[3,4-b]pyridin-4-yl)-2-fluorobenzyl)-4,4-dimethylpentanamide (944-7) (95 mg, 68%) was obtained as a yellow solid from 4-(4-(aminomethyl)-3-fluorophenyl)-N-((1r,4r)-4-((tert-butyldimethylsilyl)oxy)cyclohexyl)-1-(4-methoxybenzyl)-1H-pyrazolo[3,4-b]pyridin-3-amine (70 mg, 0.12 mmol) and 4,4-dimethylpentanoic acid (26 mg, 0.2 mmol) following a procedure adapted from Method 3.2.1. LC-MS (ESI): m/z (M+1) 702.79.

### 3.8.7 Preparation of N-(2-fluoro-4-(3-(((1r,4r)-4-hydroxycyclohexyl)amino)-1H-pyrazolo[3,4-b]pyridin-4-yl)benzyl)-4, 4-dimethylpentanamide (944)

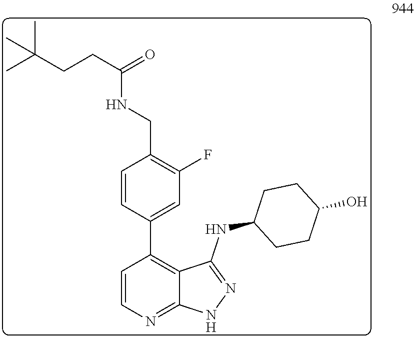 [SMILES: CC(C)(C)CCC(=O)NCc1ccc(-c2ccnc3[nH]nc(N[C@H]4CC[C@H](O)CC4)c23)cc1F]

N-(2-Fluoro-4-(3-(((1r,4r)-4-hydroxycyclohexyl)amino)-1H-pyrazolo[3,4-b]pyridin-4-yl)benzyl)-4,4-dimethylpentanamide (944) (5.8 mg, 5.6%) was obtained as a white solid from N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorobenzyl)-4,4-dimethylpentanamide (125 mg, 0.22 mmol) following a procedure adapted from Method 3.1.5. LC-MS (ESI): m/z (M+1) 468.61.

¹H NMR (400 MHz, DMSO) δ 12.40 (s, 1H), 8.51-8.35 (m, 2H), 7.59-7.34 (m, 3H), 6.93 (d, J=4.6 Hz, 1H), 4.48 (s, 1H), 4.37 (d, J=5.6 Hz, 2H), 3.82 (d, J=6.9 Hz, 1H), 3.38 (s, 2H), 2.21-2.07 (m, 2H), 1.96 (d, J=10.1 Hz, 2H), 1.74 (d, J=10.2 Hz, 2H), 1.51-1.40 (m, 2H), 1.18 (dd, J=24.0, 12.0 Hz, 2H), 1.12-1.02 (m, 2H), 0.87 (s, 9H).

### Example 17: Synthesis of 1-ethyl-N-{[2-fluoro-4-(3-{[(1r,4r)-4-hydroxycyclohexyl]amino}-1H-pyrazolo[3,4-b]pyridin-4-yl)phenyl]methyl}-1H-pyrazole-4-carboxamide (Compound-20)

### 3.9 Preparation of 1-ethyl-N-(2-fluoro-4-(3-(((1r,4r)-4-hydroxycyclohexyl)amino)-1H-pyrazolo[3,4-b]pyridin-4-yl)benzyl)-1H-pyrazole-4-carboxamide (945)

### 3.9.1 Preparation of N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)benzyl)-1-ethyl-1H-pyrazole-4-carboxamide (945-1)

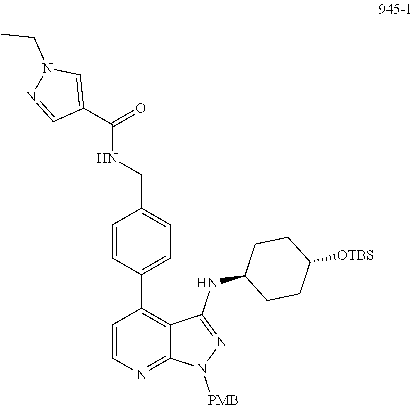

N-(4-(3-(((1r,4r)-4-((tert-Butyldimethylsilyl)oxy)cyclohexyl)amino)-1-(4-methoxybenzyl)-1H-pyrazolo[3,4-b]pyridin-4-yl)benzyl)-1-ethyl-1H-pyrazole-4-carboxamide (945-1) (64 mg, 95%) was obtained as a yellow solid from 4-(4-(aminomethyl)-3-fluorophenyl)-N-((1r,4r)-4-((tert-butyldimethylsilyl)oxy)cyclohexyl)-1-(4-methoxybenzyl)-1H-pyrazolo[3,4-b]pyridin-3-amine (55 mg, 0.093 mmol) and 1-ethyl-1H-pyrazole-4-carboxylic acid (17 mg, 0.12 mmol) following a procedure adapted from Method 3.2.1. LC-MS (ESI): m/z (M−1+46) 756.72.

### 3.9.2 Preparation of 1-ethyl-N-(2-fluoro-4-(3-(((1r,4r)-4-hydroxycyclohexyl)amino)-1H-pyrazolo[3,4-b]pyridin-4-yl)benzyl)-1H-pyrazole-4-carboxamide (945)

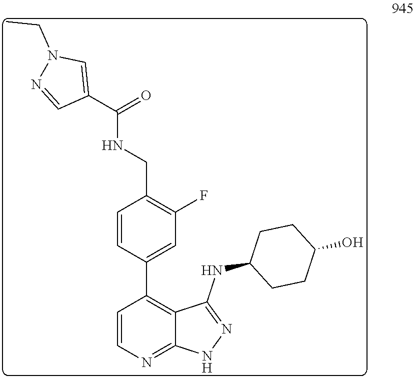 [SMILES: CCn1cc(C(=O)NCc2ccc(-c3ccnc4[nH]nc(N[C@H]5CC[C@H](O)CC5)c34)cc2F)cn1]

1-Ethyl-N-(2-fluoro-4-(3-(((1r,4r)-4-hydroxycyclohexyl)amino)-1H-pyrazolo[3,4-b]pyridin-4-yl)benzyl)-1H-pyrazole-4-carboxamide (945) (10 mg, 23%) was obtained as a white solid from N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)benzyl)-1-ethyl-1H-pyrazole-4-carboxamide (64 mg, 0.09 mmol) following a procedure adapted from Method 3.1.5. LC-MS (ESI): m/z (M+1) 478.47.

¹H NMR (400 MHz, DMSO) δ 12.39 (s, 1H), 8.66 (t, J=5.9 Hz, 1H), 8.40 (d, J=4.7 Hz, 1H), 8.22 (s, 1H), 7.90 (s, 1H), 7.52 (t, J=7.9 Hz, 1H), 7.48-7.37 (m, 2H), 6.93 (d, J=4.7 Hz, 1H), 4.53 (d, J=5.8 Hz, 2H), 4.47 (d, J=3.9 Hz, 1H), 4.15 (q, J=7.3 Hz, 2H), 3.83 (d, J=7.2 Hz, 1H), 3.35 (s, 1H), 1.95 (d, J=9.6 Hz, 2H), 1.72 (d, J=9.6 Hz, 2H), 1.38 (t, J=7.3 Hz, 3H), 1.17 (dd, J=22.5, 9.8 Hz, 2H), 1.05 (dd, J=22.7, 10.2 Hz, 2H).

### Example 18: Synthesis of 5-tert-butyl-N-{[2-fluoro-4-(3-{[(1r,4r)-4-hydroxycyclohexyl]amino}-1H-pyrazolo[3,4-b]pyridin-4-yl)phenyl]methyl}-1,2,4-oxadiazole-3-carboxamide (Compound-21)

### 3.10 Preparation of 5-(tert-butyl)-N-(2-fluoro-4-(3-(((1r,4r)-4-hydroxycyclohexyl)amino)-1H-pyrazolo[3,4-b]pyridin-4-yl)benzyl)-1,2,4-oxadiazole-3-carboxamide (949)

### 3.10.1 Preparation of ethyl 2-(hydroxyamino)-2-iminoacetate (949-2)

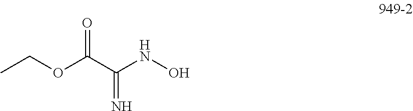

To a stirred solution of ethyl carbonocyanidate (5.0 g, 50.5 mmol) in EtOH/H₂O (50.5 mL/30 mL) were added H₂ONH₂OH.HCl (5.26 g, 75.7 mmol) and Na₂CO₃ (4.12 g, 38.9 mmol). After being stirred at room temperature overnight, the reaction mixture was partitioned between EA and H₂O. The layers were separated and the aqueous layer was extracted with EA (3×). The combined organic layers were washed with brine and dried over Na₂SO₄. The solvents were removed under vacuum to provide ethyl 2-(hydroxyamino)-2-iminoacetate (949-2) (5.05 g, 76%) as a white solid which was used in the next step without purification. LC-MS: ESI m/z (M+1)=133.25

### 3.10.2 Preparation of ethyl 5-(tert-butyl)-1,2,4-oxadiazole-3-carboxylate (949-4)

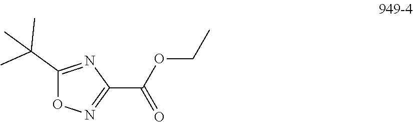

To a stirred solution of ethyl 2-(hydroxyamino)-2-iminoacetate (5.0 g, 37.8 mmol) in DMF (60 mL) were added pivalic acid (3.86 g, 37.8 mmol), 1-Ethyl-3-(3-dimethylaminopropyl)carbodiimide (EDCI) (7.97 g, 41.58 mmol), HOBt (6.12 g, 45.3 mmol) and DIPEA (19.7 mL, 113.4 mmol). After being stirred at room temperature overnight, the reaction mixture was heated up to 100° C. for hr before cooled down to room temperature and partitioned between EA and H₂O. The layers were separated and the aqueous layer was extracted with EA (3×). The combined organic layers were washed with brine and dried over Na₂SO₄. The solvents were removed and the residue was purified by flash chromatography (silica gel, 10˜90% ethyl acetate in petroleum ether) to provide ethyl 5-(tert-butyl)-1,2,4-oxadiazole-3-carboxylate (949-4) (1.8 g, 24%) as a yellow solid. LC-MS: ESI m/z (M+1)=199.36.

### 3.10.3 Preparation of 5-(tert-butyl)-1,2,4-oxadiazole-3-carboxylic acid (949-5)

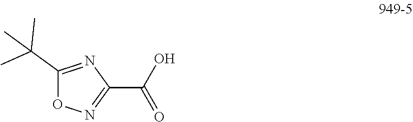

To the solution of ethyl 5-(tert-butyl)-1,2,4-oxadiazole-3-carboxylate (1.8 g, 9.1 mmol) in MeOH/H₂O (9 mL/9 mL) was added LiOH (655 mg, 27.3 mmol). After being stirred at room temperature for 24 hr, the reaction mixture was acidified to pH˜5 and concentrated to provide 5-(tert-butyl)-1,2,4-oxadiazole-3-carboxylic acid (949-5) (quant.) which was used in the next step without purification. LC-MS: ESI m/z (M+1)=171.38

### 3.10.4 Preparation of 5-(tert-butyl)-N-(2-fluoro-4-(3-(((1r,4r)-4-hydroxycyclohexyl)amino)-1H-pyrazolo[3,4-b]pyridin-4-yl)benzyl)-1,2,4-oxadiazole-3-carboxamide (949)

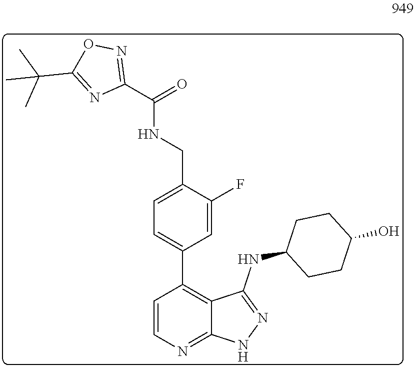 [SMILES: CC(C)(C)c1nc(C(=O)NCc2ccc(-c3ccnc4[nH]nc(N[C@H]5CC[C@H](O)CC5)c34)cc2F)no1]

5-(tert-Butyl)-N-(2-fluoro-4-(3-(((1r,4r)-4-hydroxycyclohexyl)amino)-1H-pyrazolo[3,4-b]pyridin-4-yl)benzyl)-1,2,4-oxadiazole-3-carboxamide (949) (11 mg, 20%, two steps) was obtained as a white solid from 4-(4-(aminomethyl)-3-fluorophenyl)-N-((1r,4r)-4-((tert-butyldimethylsilyl)oxy)cyclohexyl)-1-(4-methoxybenzyl)-1H-pyrazolo[3,4-b]pyridin-3-amine (65 mg, 0.11 mmol) and 5-(tert-butyl)-1,2,4-oxadiazole-3-carboxylic acid (24 mg, 0.14 mmol) following a similar procedure as outlined in Method 3.2.1, Method 3.1.5. LC-MS (ESI): m/z (M+1) 508.59. ¹H NMR (400 MHz, DMSO) δ 12.40 (s, 1H), 9.54 (t, J=6.1 Hz, 1H), 8.41 (d, J=4.7 Hz, 1H), 7.54 (t, J=7.8 Hz, 1H), 7.50-7.37 (m, 2H), 6.94 (d, J=4.7 Hz, 1H), 4.59 (d, J=5.9 Hz, 2H), 4.46 (d, J=4.3 Hz, 1H), 3.83 (d, J=7.2 Hz, 1H), 3.35 (d, J=4.3 Hz, 1H), 1.95 (d, J=10.3 Hz, 2H), 1.71 (d, J=9.2 Hz, 2H), 1.43 (s, 9H), 1.22-1.00 (m, 4H).

### Example 19: Synthesis of 4-tert-butyl-N-{1-[4-(3-{[(1r,4r)-4-hydroxycyclohexyl]amino}-1H-pyrazolo[3,4-b]pyridin-4-yl)phenyl]cyclopropyl}benzamide (Compound-22)

### 3.11 Preparation of 4-(tert-butyl)-N-(1-(4-(3-(((1r,4r)-4-hydroxycyclohexyl)amino)-1H-pyrazolo[3,4-b]pyridin-4-yl)phenyl)cyclopropyl)benzamide (985)

### 3.11.1 Preparation of N-(1-(4-bromophenyl)cyclopropyl)-4-(tert-butyl)benzamide (985-2)

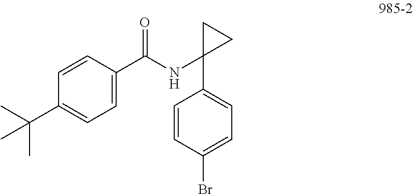 [SMILES: CC(C)(C)c1ccc(C(=O)NC2(c3ccc(Br)cc3)CC2)cc1]

N-(1-(4-Bromophenyl)cyclopropyl)-4-(tert-butyl)benzamide (985-2) (108 mg, 60%) was obtained as a white solid from 1-(4-bromophenyl)cyclopropan-1-amine (100 mg, 0.47 mmol) and 4-(tert-butyl)benzoic acid (101 mg, 0.57 mmol) following a procedure adapted from Method 3.2.1. LC-MS (ESI): m/z (MM+2) 372.48/374.49.

### 3.11.2 Preparation of 4-(tert-butyl)-N-(1-(4-(4,4,5,5-tetramethyl-1,3,2-dioxaborolan-2-yl)phenyl)cyclopropyl)benzamide (985-3)

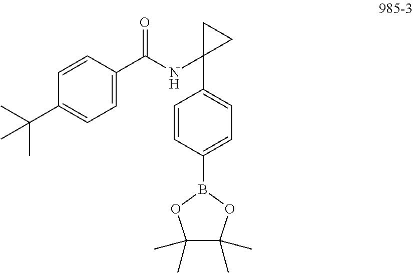 [SMILES: CC(C)(C)c1ccc(C(=O)NC2(c3ccc(B4OC(C)(C)C(C)(C)O4)cc3)CC2)cc1]

4-(tert-Butyl)-N-(1-(4-(4,4,5,5-tetramethyl-1,3,2-dioxaborolan-2-yl)phenyl)cyclopropyl)benzamide (985-3) (71 mg, 59%) was obtained as a yellow oil from N-(1-(4-bromophenyl)cyclopropyl)-4-(tert-butyl)benzamide (108 mg, 0.29 mmol) following a procedure adapted from Method 3.1.1. LC-MS (ESI): m/z (M+1) 420.77.

### 3.11.3 Preparation of 4-(tert-butyl)-N-(1-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)phenyl)cyclopropyl)benzamide (985-4)

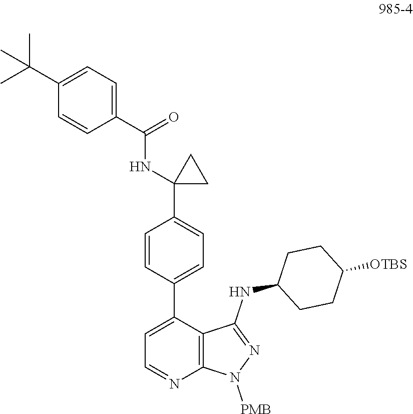

4-(tert-Butyl)-N-(1-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)phenyl)cyclopropyl)benzamide (985-4) (55 mg, 43%) was obtained as a yellow oil from 3-(((1r,4r)-4-((tert-butyldimethylsilyl)oxy)cyclohexyl)amino)-1-(4-methoxybenzyl)-1H-pyrazolo[3,4-b]pyridin-4-yl trifluoromethanesulfonate (104 mg, 0.17 mmol) and 4-(tert-butyl)-N-(1-(4-(4,4,5,5-tetramethyl-1,3,2-dioxaborolan-2-yl)phenyl)cyclopropyl)benzamide (71 mg, 0.17 mmol) following a procedure adapted from Method 3.1.4. LC-MS (ESI): m/z (M+1) 759.60.

### 3.11.4 Preparation of 4-(tert-butyl)-N-(1-(4-(3-(((1r,4r)-4-hydroxycyclohexyl)amino)-1H-pyrazolo[3,4-b]pyridin-4-yl)phenyl)cyclopropyl)benzamide (985)

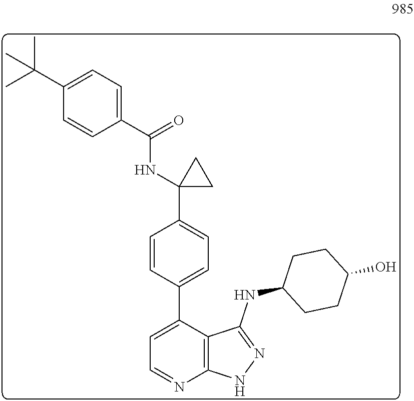 [SMILES: CC(C)(C)c1ccc(C(=O)NC2(c3ccc(-c4ccnc5[nH]nc(N[C@H]6CC[C@H](O)CC6)c45)cc3)CC2)cc1]

4-(tert-Butyl)-N-(1-(4-(3-(((1r,4r)-4-hydroxycyclohexyl)amino)-1H-pyrazolo[3,4-b]pyridin-4-yl)phenyl)cyclopropyl)benzamide (985) (20 mg, 53%) was obtained as a white solid from 4-(tert-butyl)-N-(1-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)phenyl)cyclopropyl)benzamide (55 mg, 0.072 mmol) following a procedure adapted from Method 3.1.5. LC-MS (ESI): m/z (M+1) 524.64.

¹H NMR (400 MHz, DMSO) δ 12.30 (s, 1H), 9.19 (s, 1H), 8.37 (d, J=4.7 Hz, 1H), 7.85 (d, J=8.4 Hz, 2H), 7.54-7.43 (m, 4H), 7.36 (d, J=8.3 Hz, 2H), 6.87 (d, J=4.7 Hz, 1H), 4.46 (s, 1H), 3.70 (d, J=7.2 Hz, 1H), 3.36 (s, 1H), 1.93 (d, J=12.7 Hz, 2H), 1.69 (d, J=9.7 Hz, 2H), 1.35 (s, 4H), 1.31 (s, 9H), 1.23-1.12 (m, 2H), 1.01 (dd, J=22.3, 9.8 Hz, 2H).

### Example 20: Synthesis of 4-tert-butyl-N-{[4-(3-{[(1r,4r)-4-hydroxycyclohexyl]amino}-1H-pyrazolo[3,4-b]pyridin-4-yl)phenyl]methyl}benzamide (Compound-23)

### 3.12 Preparation of 4-(tert-butyl)-N-(4-(3-(((1r,4r)-4-hydroxycyclohexyl)amino)-1H-pyrazolo[3,4-b]pyridin-4-yl)benzyl)benzamide (986)

### 3.12.1 Preparation of 4-(tert-butyl)-N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)benzyl)benzamide (986-1)

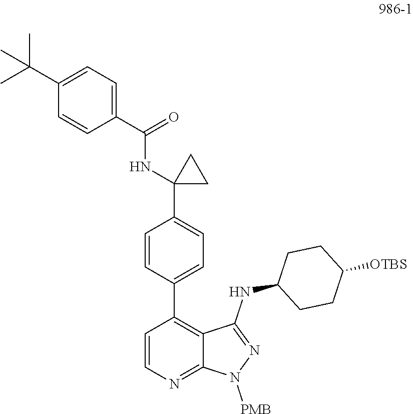

4-(tert-Butyl)-N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)benzyl)benzamide (986-1) (62 mg, 65%) was obtained as a yellow oil from 3-(((1r,4r)-4-((tert-butyldimethylsilyl)oxy)cyclohexyl)amino)-1-(4-methoxybenzyl)-1H-pyrazolo[3,4-b]pyridin-4-yl trifluoromethanesulfonate (80 mg, 0.13 mmol) and 4-(tert-butyl)-N-(4-(4,4,5,5-tetramethyl-1,3,2-dioxaborolan-2-yl)benzyl)benzamide (77 mg, 0.19 mmol) following a procedure adapted from Method 3.1.4. LC-MS (ESI): m/z (M+1) 733.00.

### 3.12.2 Preparation of 4-(tert-butyl)-N-(4-(3-(((1r,4r)-4-hydroxycyclohexyl)amino)-1H-pyrazolo[3,4-b]pyridin-4-yl)benzyl)benzamide (986)

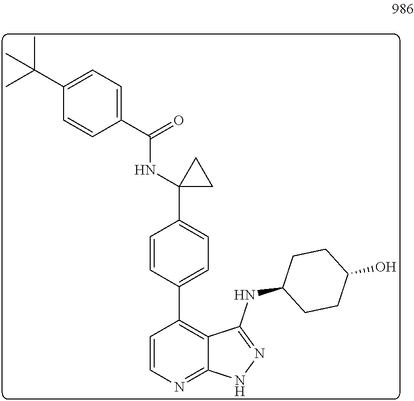 [SMILES: CC(C)(C)c1ccc(C(=O)NC2(c3ccc(-c4ccnc5[nH]nc(N[C@H]6CC[C@H](O)CC6)c45)cc3)CC2)cc1]

4-(tert-Butyl)-N-(4-(3-(((1r,4r)-4-hydroxycyclohexyl)amino)-1H-pyrazolo[3,4-b]pyridin-4-yl)benzyl)benzamide (986) (17 mg, 40%) was obtained as a white solid from 4-(tert-butyl)-N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)benzyl)benzamide (62 mg, 0.084 mmol) following a procedure adapted from Method 3.1.5. LC-MS (ESI): m/z (M+1) 498.76. ¹H NMR (400 MHz, DMSO) δ 12.32 (s, 1H), 9.06 (t, J=6.0 Hz, 1H), 8.38 (d, J=4.7 Hz, 1H), 7.85 (d, J=8.4 Hz, 2H), 7.58-7.44 (m, 6H), 6.88 (d, J=4.7 Hz, 1H), 4.57 (d, J=6.0 Hz, 2H), 4.46 (s, 1H), 3.68 (d, J=7.1 Hz, 1H), 1.95 (d, J=10.0 Hz, 2H), 1.70 (d, J=9.7 Hz, 2H), 1.31 (s, 9H), 1.22-1.11 (m, 2H), 0.99 (dd, J=22.7, 9.8 Hz, 2H).

### Example 21: Synthesis of N-{[2-fluoro-4-(3-{[(1r,4r)-4-hydroxycyclohexyl]amino}-1H-pyrazolo[3,4-b]pyridin-4-yl)phenyl]methyl}-4-(2-hydroxypropan-2-yl)benzamide (Compound-26)

### 3.13 Preparation of N-(2-fluoro-4-(3-(((1r,4r)-4-hydroxycyclohexyl)amino)-1H-pyrazolo[3,4-b]pyridin-4-yl)benzyl)-4-(2-hydroxypropan-2-yl)benzamide (941)

### 3.13.1 Preparation of tert-butyl (4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorobenzyl)carbamate (941-1)

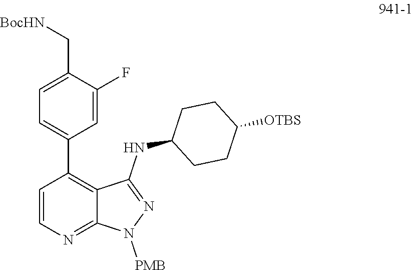

tert-Butyl (4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorobenzyl)carbamate (941-1) (466 mg, 27%) was obtained as a yellow oil from 3-(((1r,4r)-4-((tert-butyldimethylsilyl)oxy)cyclohexyl)amino)-1-(4-methoxybenzyl)-1H-pyrazolo[3,4-b]pyridin-4-yl trifluoromethanesulfonate (1.52 g, 2.47 mmol) and tert-butyl (2-fluoro-4-(4,4,5,5-tetramethyl-1,3,2-dioxaborolan-2-yl)benzyl)carbamate (1.74 g, 4.95 mmol) following a procedure adapted from Method 3.1.4. LC-MS (ESI): m/z (M+1) 691.00.

### 3.13.2 Preparation of (1r,4r)-4-((4-(4-(aminomethyl)-3-fluorophenyl)-1H-pyrazolo[3,4-b]pyridin-3-yl)amino)cyclohexan-1-ol (941-2)

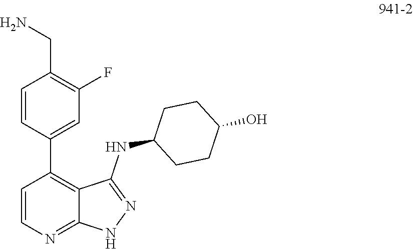 [SMILES: NCc1ccc(-c2ccnc3[nH]nc(N[C@H]4CC[C@H](O)CC4)c23)cc1F]

(1r,4r)-4-((4-(4-(Aminomethyl)-3-fluorophenyl)-1H-pyrazolo[3,4-b]pyridin-3-yl)amino)cyclohexan-1-ol (941-2) (97 mg, quant.) was obtained as a yellow solid from tert-butyl (4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorobenzyl)carbamate (120 mg, 0.17 mmol) following a similar procedure outlined in Methods 3.5.4 and 3.1.5. LC-MS (ESI): m/z (M+1) 356.18.

### 3.13.3 Preparation of 4-(2-hydroxypropan-2-yl)benzoic acid (941-4)

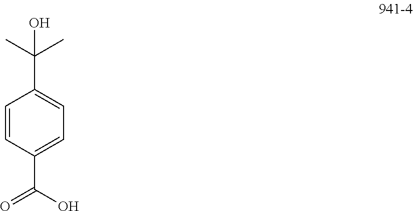

To a stirred solution of 4-isopropylbenzoic acid (1.0 g, 6 mmol) in H₂O (50 mL) were added KOH (840 mg, 15 mmol) and KMnO₄ (2.37 g, 15 mmol). The mixture was stirred at 60° C. overnight before cooled to 0° C. and treated with ethylene glycol (3 mL). The precipitate was removed by filtration and the filtrate was acidified to pH˜1 by 6N HCl and extracted with EA (20 mL×3). The combined organic layers were washed with brine, dried over Na₂SO₄, filtered to provide 4-(2-hydroxypropan-2-yl)benzoic acid (941-4) (960 mg, 88%, ˜82% purity) as a white solid which was used in the next step without further purification. LC-MS (ESI): m/z (M+1) 181.08.

### 3.13.4 Preparation of N-(2-fluoro-4-(3-(((1r,4r)-4-hydroxycyclohexyl)amino)-1H-pyrazolo[3,4-b]pyridin-4-yl)benzyl)-4-(2-hydroxypropan-2-yl)benzamide (941)

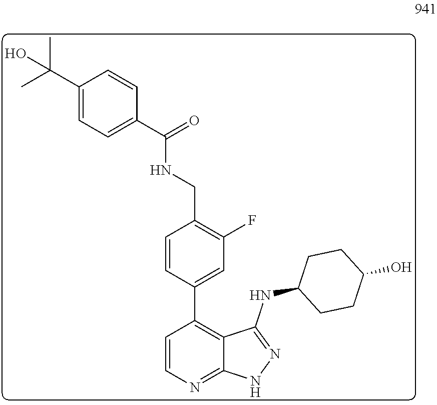 [SMILES: CC(C)(O)c1ccc(C(=O)NCc2ccc(-c3ccnc4[nH]nc(N[C@H]5CC[C@H](O)CC5)c34)cc2F)cc1]

N-(2-Fluoro-4-(3-(((1r,4r)-4-hydroxycyclohexyl)amino)-1H-pyrazolo[3,4-b]pyridin-4-yl)benzyl)-4-(2-hydroxypropan-2-yl)benzamide (941) (13 mg, 9%) was obtained as a yellow solid from (1r,4r)-4-((4-(4-(aminomethyl)-3-fluorophenyl)-1H-pyrazolo[3,4-b]pyridin-3-yl)amino)cyclohexan-1-ol (97 mg, 0.27 mmol) and 4-(2-hydroxypropan-2-yl)benzoic acid (49 mg, 0.27 mmol) following a procedure adapted from Method 3.2.1. LC-MS (ESI): m/z (M+1) 518.51. ¹H NMR (400 MHz, DMSO) δ 12.38 (s, 1H), 9.05 (t, J=5.7 Hz, 1H), 8.40 (d, J=4.7 Hz, 1H), 7.85 (d, J=8.5 Hz, 2H), 7.56 (d, J=8.5 Hz, 2H), 7.52 (d, J=7.8 Hz, 1H), 7.45 (d, J=10.9 Hz, 1H), 7.39 (d, J=7.8 Hz, 1H), 6.93 (d, J=4.7 Hz, 1H), 5.12 (s, 1H), 4.59 (d, J=5.8 Hz, 2H), 4.47 (d, J=4.4 Hz, 1H), 3.81 (d, J=7.1 Hz, 1H), 2.05-1.90 (m, J=14.5, 6.9 Hz, 4H), 1.71 (d, J=9.0 Hz, 2H), 1.44 (s, 6H), 1.20-1.10 (m, 2H), 1.10-0.98 (m, 2H).

### Example 22: Synthesis of 5-tert-butyl-N-{[2-fluoro-4-(3-{[(1r,4r)-4-hydroxycyclohexyl]amino}-1H-pyrazolo[3,4-b]pyridin-4-yl)phenyl]methyl}-1,2-oxazole-3-carboxamide (Compound-27)

### 3.14 Preparation of 5-(tert-butyl)-N-(2-fluoro-4-(3-(((1r,4r)-4-hydroxycyclohexyl)amino)-1H-pyrazolo[3,4-b]pyridin-4-yl)benzyl)isoxazole-3-carboxamide (947)

### 3.14.1 Preparation of 5-(tert-butyl)-N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorobenzyl)isoxazole-3-carboxamide (947-1)

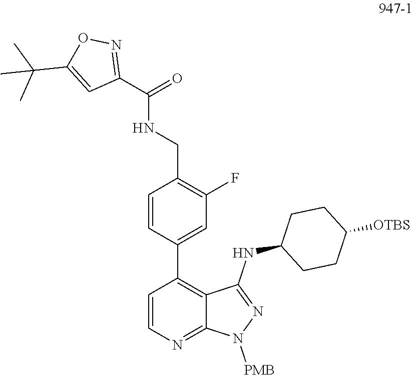

5-(tert-Butyl)-N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorobenzyl)isoxazole-3-carboxamide (947-1) (65 mg, 86%) was obtained as a yellow solid from 4-(4-(aminomethyl)-3-fluorophenyl)-N-((1r,4r)-4-((tert-butyldimethyl silyl)oxy)cyclohexyl)-1-(4-methoxybenzyl)-1H-pyrazolo[3,4-b]pyridin-3-amine (60 mg, 0.1 mmol) and 5-(tert-butyl)isoxazole-3-carboxylic acid (22 mg, 0.13 mmol) following a procedure adapted from Method 3.2.1. LC-MS (ESI): m/z (M+1) 741.63.

### 3.14.2 Preparation of 5-(tert-butyl)-N-(2-fluoro-4-(3-(((1r,4r)-4-hydroxycyclohexyl)amino)-1H-pyrazolo[3,4-b]pyridin-4-yl)benzyl)isoxazole-3-carboxamide (947)

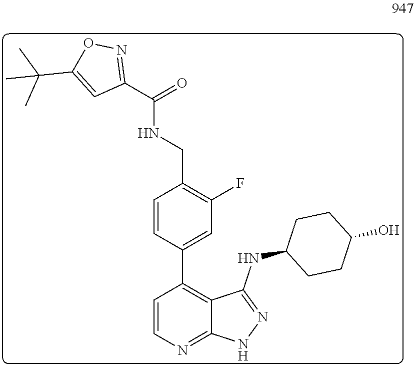 [SMILES: CC(C)(C)c1cc(C(=O)NCc2ccc(-c3ccnc4[nH]nc(N[C@H]5CC[C@H](O)CC5)c34)cc2F)no1]

5-(tert-Butyl)-N-(2-fluoro-4-(3-(((1r,4r)-4-hydroxycyclohexyl)amino)-1H-pyrazolo[3,4-b]pyridin-4-yl)benzyl)isoxazole-3-carboxamide (947) (25 mg, 56%) was obtained as a white solid from 5-(tert-butyl)-N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorobenzyl)isoxazole-3-carboxamide (65 mg, 0.088 mmol) following a procedure adapted from Method 3.1.5. LC-MS (ESI): m/z (M+1) 507.6.

¹H NMR (400 MHz, DMSO) δ 12.39 (s, 1H), 9.33 (t, J=6.0 Hz, 1H), 8.41 (d, J=4.7 Hz, 1H), 7.53 (t, J=7.8 Hz, 1H), 7.45 (dd, J=10.8, 1.5 Hz, 1H), 7.40 (dd, J=7.8, 1.6 Hz, 1H), 6.94 (d, J=4.7 Hz, 1H), 6.58 (s, 1H), 4.57 (d, J=6.0 Hz, 2H), 4.46 (d, J=4.2 Hz, 1H), 3.82 (d, J=7.2 Hz, 1H), 3.36 (s, 1H), 1.95 (d, J=9.8 Hz, 2H), 1.71 (d, J=10.2 Hz, 2H), 1.33 (s, 9H), 1.21-1.12 (m, 2H), 1.05 (dd, J=22.8, 10.0 Hz, 2H).

### Example 23: Synthesis of 4-cyclopropyl-N-{[2-fluoro-4-(3-{[(3R)-pyrrolidin-3-yl]amino}-1H-pyrazolo[3,4-b]pyridin-4-yl)phenyl]methyl}benzamide (Compound-35)

Using a similar procedure to Example 1 (Compound 2), Compound 35 was prepared: LC-MS (ESI): m/z (M+1) 471.63. ¹H NMR (400 MHz, DMSO) δ 12.56 (s, 1H), 9.06 (t, J=5.7 Hz, 1H), 8.43 (d, J=4.7 Hz, 1H), 8.34 (s, 1H), 7.81 (d, J=8.3 Hz, 2H), 7.55-7.44 (m, 3H), 7.17 (d, J=8.3 Hz, 2H), 6.99 (d, J=4.7 Hz, 1H), 4.63-4.49 (m, 3H), 4.12 (s, 1H), 3.20-3.16 (m, 1H), 3.08-2.92 (m, 3H), 2.12-2.04 (m, 1H), 2.00-1.94 (m, 1H), 1.71-1.59 (m, 1H), 1.01 (td, J=6.4, 4.2 Hz, 2H), 0.78-0.70 (m, 2H).

### Example 24: Synthesis of 4-cyclopropyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}benzamide (Compound-40)

Using a similar procedure as in Example 2 (Compound 3), Compound 40 was made: LC-MS (ESI): m/z (M+1) 525.64 (calculated exact mass: 524.23). ¹H NMR (400 MHz, DMSO) δ 12.57 (s, 1H), 9.00 (d, J=6.4 Hz, 1H), 8.43 (d, J=4.8 Hz, 1H), 7.81 (dd, J=8.3, 3.9 Hz, 2H), 7.55-7.45 (m, 2H), 7.43-7.37 (m, 1H), 7.18 (dd, J=8.3, 4.1 Hz, 2H), 6.99 (dd, J=4.7, 1.2 Hz, 1H), 6.54-6.45 (m, 1H), 6.14-6.06 (m, 1H), 5.63-5.55 (m, 1H), 4.57 (d, J=5.6 Hz, 2H), 4.49 (dd, J=14.3, 5.5 Hz, 1H), 4.19-4.10 (m, 1H), 3.85-3.35 (m, 4H), 2.16-2.07 (m, 1H), 2.02-1.95 (m, 1H), 1.88-1.78 (m, 1H), 1.04-0.97 (m, 2H), 0.74 (dt, J=6.6, 4.3 Hz, 2H).

### Example 25: Synthesis of 4-cyclopropyl-N-{[2-fluoro-4-(3-{[(3R)-1-(3-methyloxetane-3-carbonyl)pyrrolidin-3-yl]amino}-1H-pyrazolo[3,4-b]pyridin-4-yl)phenyl]methyl}benzamide (Compound-41)

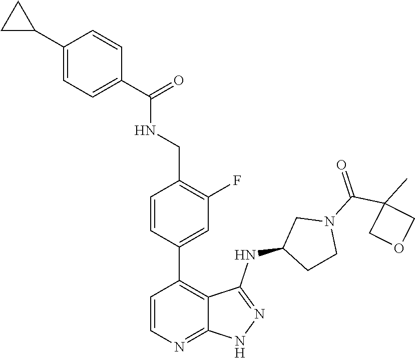 [SMILES: CC1(C(=O)N2CC[C@@H](Nc3n[nH]c4nccc(-c5ccc(CNC(=O)c6ccc(C7CC7)cc6)c(F)c5)c34)C2)COC1]

Using a similar procedure as in Example 1 (Compound 2) (Method C), Compound 41 was made: LC-MS (ESI): m/z (M+1) 569.62 (calculated exact mass: 568.21). ¹H NMR (400 MHz, DMSO) δ 12.57 (d, J=8.6 Hz, 1H), 9.00 (dt, J=18.8, 5.8 Hz, 1H), 8.43 (d, J=4.7 Hz, 1H), 7.81 (dd, J=8.3, 1.6 Hz, 2H), 7.54-7.36 (m, 3H), 7.18 (dd, J=8.4, 2.3 Hz, 2H), 6.99 (d, J=4.7 Hz, 1H), 4.82-4.61 (m, 1H), 4.78 (t, J=5.6 Hz, 1H), 4.57 (d, J=5.1 Hz, 2H), 4.47 (dd, J=12.0, 5.0 Hz, 1H), 4.17-4.05 (m, 3H), 3.56-3.04 (m, 4H), 2.14-1.95 (m, 2H), 1.90-1.79 (m, 1H), 1.44 (d, J=38.3 Hz, 3H), 1.05-0.97 (m, 2H), 0.79-0.70 (m, 2H).

### Example 26: Synthesis of 6-tert-butyl-2-{[2-fluoro-4-(3-{[(11r,4r)-4-hydroxycyclohexyl]amino}-1H-pyrazolo[3,4-b]pyridin-4-yl)phenyl]methyl}-1,2,3,4-tetrahydroisoquinolin-1-one (Compound-38)

### 3.15 Preparation of 6-(tert-butyl)-2-(2-fluoro-4-(3-(((1r,4r)-4-hydroxycyclohexyl)amino)-1H-pyrazolo[3,4-b]pyridin-4-yl)benzyl)-3,4-dihydroisoquinolin-1(2H)-one (940)

### 3.15.1 Preparation of 1-(4-(tert-butyl)phenyl)-3-chloropropan-1-one (940-2)

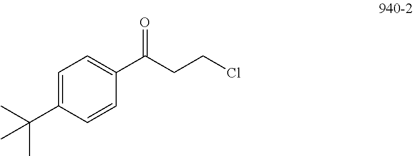

At 0° C. under N₂, to a solution of tert-butylbenzene (5 g, 37.31 mmol) and Aluminum chloride (5.41 g, 41.04 mmol) in dry DCM (56 mL) was added 3-chloropropanoyl chloride (4.46 g, 37.31 mmol, in 15 mL DCM). After being stirred at room temperature for 3 hrs, the reaction mixture was quenched with water (20 mL) at 0° C., extracted with DCM (20 mL×2). The combined organic layers were washed with water (20 mL×2), dried, filtered and concentrated to give the residue which was purified by flash chromatography (silica gel, 0˜10% ethyl acetate in petroleum ether) to afford the product 1-(4-(tert-butyl)phenyl)-3-chloropropan-1-one (940-2) (6 g, 72%).

LC-MS (ESI): m/z (M/M+2) 225.31/227.32.

### 3.15.2 Preparation of 5-(tert-butyl)-2,3-dihydro-1H-inden-1-one (940-3)

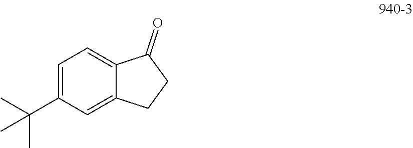

A mixture of 1-(4-(tert-butyl)phenyl)-3-chloropropan-1-one (6.0 g, 26.7 mmol) in conc. H₂SO₄(37 mL) was stirred at room temperature for 3 hrs before poured into ice water (20 mL), extracted with DCM (50 mL×2). The combined organic layers were washed with water (20 mL×2), dried, filtered and concentrated to give the residue which was purified by flash chromatography (silica gel, 0˜10% ethyl acetate in petroleum ether) to afford the product 5-(tert-butyl)-2,3-dihydro-1H-inden-1-one (940-3) (3 g, 60%). LC-MS (ESI): m/z (M+1) 189.36.

### 3.15.3 Preparation of 6-(tert-butyl)-3,4-dihydroisoquinolin-1(2H)-one (940-4)

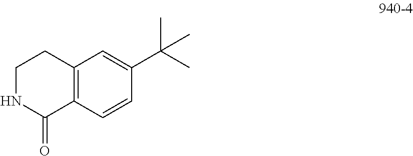

To a solution of 5-(tert-butyl)-2,3-dihydro-1H-inden-1-one (1.0 g, 5.3 mmol) in dry DCM (10 mL) and methanesulfonicacid (6 mL) was added sodium azide (690 mg, 10.6 mmol) at 0° C. under N₂. After being stirred at room temperature for 2 hrs, the reaction mixture was quenched with sat.NaHCO₃ (20 mL) at 0° C., extracted with DCM (20 mL×2). The combined organic layers were washed with water (10 mL×2), dried, filtered and concentrated to give the residue which was purified by flash chromatography (silica gel, 0˜10% ethyl acetate in petroleum ether) to afford the product 6-(tert-butyl)-3,4-dihydroisoquinolin-1 (2H)-one (940-4) (600 mg, 56.1%).

LC-MS (ESI): m/z (M+1) 204.36.

### 3.15.4 Preparation of 2-(4-bromo-2-fluorobenzyl)-6-(tert-butyl)-3,4-dihydroisoquinolin-1(2H)-one (940-5)

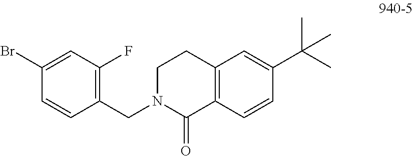 [SMILES: CC(C)(C)c1ccc2c(c1)CCN(Cc1ccc(Br)cc1F)C2=O]

At 0° C. under N₂, to a stirred solution of 6-(tert-butyl)-3,4-dihydroisoquinolin-1(2H)-one (200 mg, 0.985 mmol) and 4-bromo-1-(bromomethyl)-2-fluorobenzene (340 mg, 1.28 mmol) in dry DMF (4 mL) was added NaH (51.2 mg, 1.28 mmol). After being stirred at room temperature for 3 hr, the reaction mixture was quenched with water (50 mL) at 0° C., extracted with DCM (10 mL×2). The combined organic layers were washed with water (10 mL×2), dried, filtered and concentrated to give the residue which was purified by flash chromatography (silica gel, 0˜20% ethyl acetate in petroleum ether) to afford the product 2-(4-bromo-2-fluorobenzyl)-6-(tert-butyl)-3,4-dihydroisoquinolin-1(2H)-one (940-5) (242 mg, 63%). LC-MS (ESI): m/z (M/M+2) 390.44/392.42.

### 3.15.5 Preparation of 6-(tert-butyl)-2-(2-fluoro-4-(4,4,5,5-tetramethyl-1,3,2-dioxaborolan-2-yl)benzyl)-3,4-dihydroisoquinolin-1 (2H)-one (940-6)

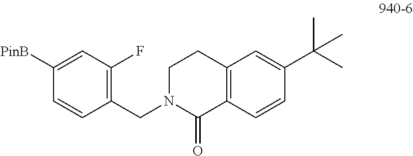

6-(tert-Butyl)-2-(2-fluoro-4-(4,4,5,5-tetramethyl-1,3,2-dioxaborolan-2-yl)benzyl)-3,4-dihydroisoquinolin-1(2H)-one (940-6) (130 mg, 83%) was obtained as a white solid from 2-(4-bromo-2-fluorobenzyl)-6-(tert-butyl)-3,4-dihydroisoquinolin-1(2H)-one (150 mg, 0.386 mmol) following a procedure adapted from Method 3.1.1. LC-MS (ESI): m/z 438.57.

### 3.15.6 Preparation of 6-(tert-butyl)-2-(4-(3-(((1r,4r)-4-((tert-butyldimethylsilyl)oxy)cyclohexyl)amino)-1-(4-methoxybenzyl)-1H-pyrrolo[2,3-b]pyridin-4-yl)-2-fluorobenzyl)-3, 4-dihydroisoquinolin-1 (2H)-one (940-7)

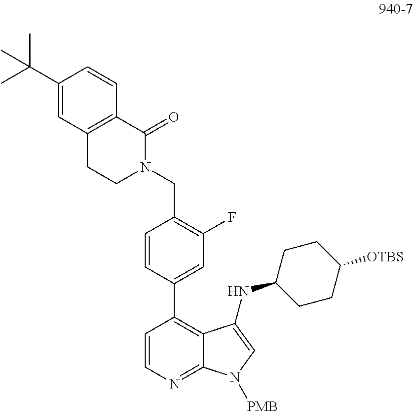

6-(tert-Butyl)-2-(4-(3-(((1r,4r)-4-((tert-butyldimethylsilyl)oxy)cyclohexyl)amino)-1-(4-methoxybenzyl)-1H-pyrrolo[2,3-b]pyridin-4-yl)-2-fluorobenzyl)-3,4-dihydroisoquinolin-1(2H)-one (940-7) (80 mg, 64%) was obtained as a yellow oil from 3-(((1r,4r)-4-((tert-butyldimethylsilyl)oxy)cyclohexyl)amino)-1-(4-methoxybenzyl)-1H-pyrazolo[3,4-b]pyridin-4-yl trifluoromethanesulfonate (129 mg, 0.16 mmol) and 6-(tert-butyl)-2-(2-fluoro-4-(4,4,5,5-tetramethyl-1,3,2-dioxaborolan-2-yl)benzyl)-3,4-dihydroisoquinolin-1(2H)-one (130 mg, 0.32 mmol) following a procedure adapted from Method 3.1.4. LC-MS (ESI): m/z (M+1) 776.77.

### 3.15.7 Preparation of 6-(tert-butyl)-2-(2-fluoro-4-(3-(((1r,4r)-4-hydroxycyclohexyl)amino)-1H-pyrazolo[3,4-b]pyridin-4-yl)benzyl)-3, 4-dihydroisoquinolin-1 (2H)-one (940)

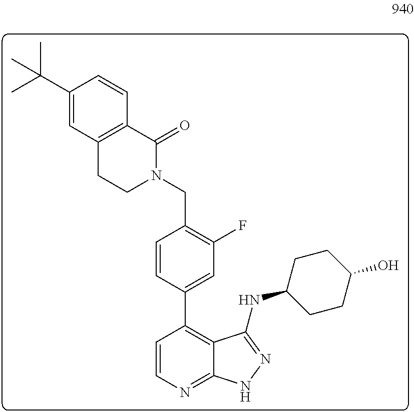 [SMILES: CC(C)(C)c1ccc2c(c1)CCN(Cc1ccc(-c3ccnc4[nH]nc(N[C@H]5CC[C@H](O)CC5)c34)cc1F)C2=O]

6-(tert-Butyl)-2-(2-fluoro-4-(3-(((1r,4r)-4-hydroxycyclohexyl)amino)-1H-pyrazolo[3,4-b]pyridin-4-yl)benzyl)-3,4-dihydroisoquinolin-1(2H)-one (940) (21 mg, 37%) was obtained as a white solid from 6-(tert-butyl)-2-(4-(3-(((1r,4r)-4-((tert-butyldimethylsilyl)oxy)cyclohexyl)amino)-1-(4-methoxybenzyl)-1H-pyrrolo[2,3-b]pyridin-4-yl)-2-fluorobenzyl)-3,4-dihydroisoquinolin-1(2H)-one (80 mg, 0.1 mmol) following a procedure adapted from Method 3.1.5. LC-MS (ESI): m/z (M+1) 542.75. ¹H NMR (400 MHz, DMSO) δ 12.40 (s, 1H), 8.40 (d, J=4.7 Hz, 1H), 7.85 (d, J=8.2 Hz, 1H), 7.47 (ddd, J=31.2, 13.8, 7.1 Hz, 4H), 7.33 (s, 1H), 6.94 (d, J=4.7 Hz, 1H), 4.84 (s, 2H), 4.45 (s, 1H), 3.85 (d, J=7.0 Hz, 1H), 3.59 (t, J=6.3 Hz, 2H), 3.30-3.19 (m, 2H), 3.02 (t, J=6.2 Hz, 2H), 1.93 (d, J=11.7 Hz, 2H), 1.70 (d, J=9.4 Hz, 2H), 1.30 (s, 9H), 1.18-0.97 (m, 4H).

### Example 27: Synthesis of 2-tert-butyl-N-{[2-fluoro-4-(3-{[(1r,4r)-4-hydroxycyclohexyl]amino}-1H-pyrazolo[3,4-b]pyridin-4-yl)phenyl]methyl}-1,3-oxazole-5-carboxamide (Compound-39)

### 3.16 Preparation of 2-(tert-butyl)-N-(2-fluoro-4-(3-(((1r,4r)-4-hydroxycyclohexyl)amino)-1H-pyrazolo[3,4-b]pyridin-4-yl)benzyl)oxazole-5-carboxamide (946)

### 3.16.1 Preparation of ethyl 2-(tert-butyl)oxazole-5-carboxylate (946-2)

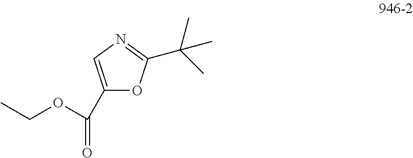

A solution of ethyl pyruvate (2.0 g, 17.2 mmol) and HDNIB (9.7 g, 20.7 mmol) in trimethylacetonitrile (15 mL) was heated to reflux for 3 hrs. After the reaction mixture was cooled to room temperature, 2,6-lutidine (0.2 mL, 1.7 mmol) was added. The reaction mixture was refluxed for an additional 8 hrs. The reaction was checked by LC-MS and the solvent was removed. The residue was dissolved in CH₂Cl₂, washed with water and brine, dried (Na₂SO₄), concentrated in vacuo and purified via column chromatography (silica gel, 0˜20% EA in PE) to obtain ethyl 2-(tert-butyl)oxazole-5-carboxylate (946-2) (400 mg, 12%). LC-MS (ESI): m/z (M+1) 198.11.

### 3.16.2 Preparation of 2-(tert-butyl)oxazole-5-carboxylic acid (946-3)

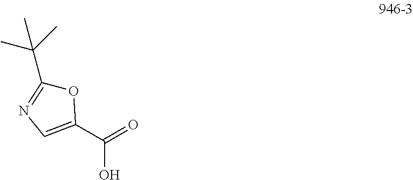

At room temperature, to a stirred solution of ethyl 2-(tert-butyl)oxazole-5-carboxylate (400 mg, 2.0 mmol) in a mixture of EtOH (5 ml) and water (1 mL) was added NaOH (400 mg, 10 mmol). The mixture was stirred at 80° C. for 6 hr. The resulting solution was acidified to pH 1 by addition of 6 N HCl solution and then the mixture was extracted with EA (20 mL×3). The combined organic layers were washed with brine, dried over Na₂SO₄, filtered to provide the crude 2-(tert-butyl)oxazole-5-carboxylic acid (946-3) (330 mg, 97%) as a yellow oil without further purification. LC-MS (ESI): m/z (M+1) 170.07.

### 3.16.3 Preparation of 2-(tert-butyl)-N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorobenzyl)oxazole-5-carboxamide (946-4)

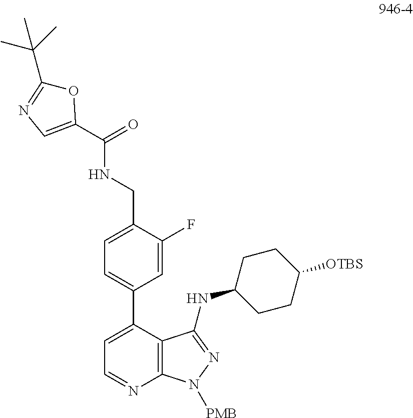

2-(tert-Butyl)-N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorobenzyl)oxazole-5-carboxamide (946-4) (298 mg, quant.) was obtained as a yellow solid from 4-(4-(aminomethyl)-3-fluorophenyl)-N-((1r,4r)-4-((tert-butyldimethyl silyl)oxy)cyclohexyl)-1-(4-methoxybenzyl)-1H-pyrazolo[3,4-b]pyridin-3-amine (100 mg, 0.17 mmol) and 2-(tert-butyl)oxazole-5-carboxylic acid (89 mg, 0.53 mmol) following a procedure adapted from Method 3.2.1, 3.2.2, and 3.2.3. LC-MS (ESI): m/z (M+1) 603.65.

### 3.16.4 Preparation of 2-(tert-butyl)-N-(2-fluoro-4-(3-(((1r,4r)-4-hydroxycyclohexyl)amino)-1H-pyrazolo[3,4-b]pyridin-4-yl)benzyl)oxazole-5-carboxamide (946)

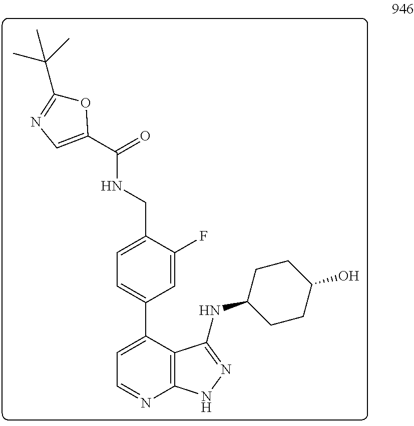 [SMILES: CC(C)(C)c1ncc(C(=O)NCc2ccc(-c3ccnc4[nH]nc(N[C@H]5CC[C@H](O)CC5)c34)cc2F)o1]

2-(tert-Butyl)-N-(2-fluoro-4-(3-(((1r,4r)-4-hydroxycyclohexyl)amino)-1H-pyrazolo[3,4-b]pyridin-4-yl)benzyl)oxazole-5-carboxamide (946) (4.8 mg, 2.4%) was obtained as a white solid from 2-(tert-butyl)-N-(4-(3-(((1r,4r)-4-((tert-butyldimethylsilyl)oxy)cyclohexyl)amino)-1-(4-methoxybenzyl)-1H-pyrazolo[3,4-b]pyridin-4-yl)-2-fluorobenzyl)oxazole-5-carboxamide (290 mg, 0.39 mmol) following a procedure adapted from Method 3.1.5. LC-MS (ESI): m/z (M+1) 507.57.

¹H NMR (400 MHz, CDCl3) δ 8.45 (d, J=4.0 Hz, 1H), 8.15 (s, 1H), 7.59 (t, J=7.7 Hz, 1H), 7.43 (t, J=6.2 Hz, 1H), 7.33-7.27 (m, 2H), 6.86 (d, J=4.8 Hz, 1H), 4.76 (d, J=6.3 Hz, 2H), 3.66-3.55 (m, 2H), 2.15 (d, J=11.3 Hz, 2H), 1.93 (d, J=10.3 Hz, 2H), 1.47-1.37 (m, 11H), 1.17-1.09 (m, 2H).

### Example 28: Synthesis of 6-tert-butyl-2-{[2-fluoro-4-(3-{[(3R)-pyrrolidin-3-yl]amino}-1H-pyrazolo[3,4-b]pyridin-4-yl)phenyl]methyl}-1,2,3,4-tetrahydroisoquinolin-1-one (Compound-36)

### 3.2. Preparation of (R)-6-(tert-butyl)-2-(2-fluoro-4-(3-(pyrrolidin-3-ylamino)-1H-pyrazolo[3,4-b]pyridin-4-yl)benzyl)-3,4-dihydroisoquinolin-1 (2H)-one (1015)

### 3.3.5. Preparation of (R)-6-(tert-butyl)-2-(2-fluoro-4-(3-(pyrrolidin-3-ylamino)-1H-pyrazolo[3,4-b]pyridin-4-yl)benzyl)-3,4-dihydroisoquinolin-1 (2H)-one (1015)

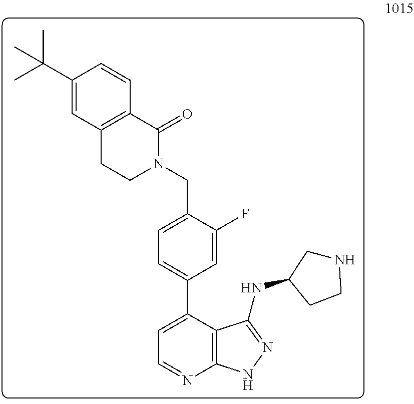 [SMILES: CC(C)(C)c1ccc2c(c1)CCN(Cc1ccc(-c3ccnc4[nH]nc(N[C@@H]5CCNC5)c34)cc1F)C2=O]

(R)-6-(tert-Butyl)-2-(2-fluoro-4-(3-(pyrrolidin-3-ylamino)-1H-pyrazolo[3,4-b]pyridin-4-yl)benzyl)-3,4-dihydroisoquinolin-1(2H)-one (1015) (30 mg, 7%) was obtained as a yellow solid from tert-butyl (R)-3-((1-(4-methoxybenzyl)-4-(((trifluoromethyl)sulfonyl)oxy)-1H-pyrazolo[3,4-b]pyridin-3-yl)amino)pyrrolidine-1-carboxylate (461 mg, 0.80 mmol) and 6-(tert-butyl)-2-(2-fluoro-4-(4,4,5,5-tetramethyl-1,3,2-dioxaborolan-2-yl)benzyl)-3,4-dihydroisoquinolin-1(2H)-one (300 mg, 0.97 mmol) following a procedure adapted from that for 934, Method 3.1.4. & Method 3.1.5. LC-MS (ESI): m/z (M+1) 513.69. ¹H NMR (400 MHz, DMSO) δ 12.64-12.46 (s, 1H), 8.43 (dd, J=4.7, 2.5 Hz, 1H), 8.38 (s, 1H), 7.86 (dd, J=8.2, 2.3 Hz, 1H), 7.57-7.39 (m, 4H), 7.33 (s, 1H), 7.00 (dd, J=4.7, 2.4 Hz, 1H), 4.84 (s, 2H), 4.51 (d, J=4.4 Hz, 1H), 4.08 (s, 1H), 3.59 (d, J=4.7 Hz, 2H), 3.10 (s, 1H), 3.03-2.85 (m, 5H), 2.04 (s, 1H), 1.58 (s, 1H), 1.30 (d, J=2.4 Hz, 9H).

### Example 29: Synthesis of 6-tert-butyl-2-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,2,3,4-tetrahydroisoquinolin-1-one (Compound-43)

(R)-2-(4-(3-((1-acryloylpyrrolidin-3-yl)amino)-1H-pyrazolo[3,4-b]pyridin-4-yl)-2-fluorobenzyl)-6-(tert-butyl)-3,4-dihydroisoquinolin-1(2H)-one was obtained from (R)-6-(tert-butyl)-2-(2-fluoro-4-(3-(pyrrolidin-3-ylamino)-1H-pyrazolo[3,4-b]pyridin-4-yl)benzyl)-3,4-dihydroisoquinolin-1(2H)-one in accordance with Method E.

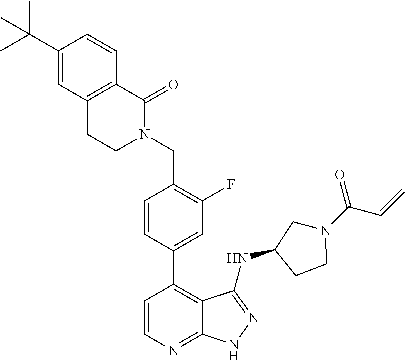 [SMILES: C=CC(=O)N1CC[C@@H](Nc2n[nH]c3nccc(-c4ccc(CN5CCc6cc(C(C)(C)C)ccc6C5=O)c(F)c4)c23)C1]

free-base; MH⁺=567.2.

### Example 30: Synthesis of 4-tert-butyl-N-{[2-fluoro-4-(3-{[(3S)-pyrrolidin-3-yl]amino}-1H-pyrazolo[3,4-b]pyridin-4-yl)phenyl]methyl}benzamide (Compound-37)

### 3.17 Preparation of (S)-4-(tert-butyl)-N-(2-fluoro-4-(3-(pyrrolidin-3-ylamino)-1H-pyrazolo[3,4-b]pyridin-4-yl)benzyl)benzamide (1021)

### 3.17.1 Preparation of tert-butyl (S)-3-((4-hydroxy-1-(4-methoxybenzyl)-1H-pyrazolo[3,4-b]pyridin-3-yl)amino)pyrrolidine-1-carboxylate (1021-2)

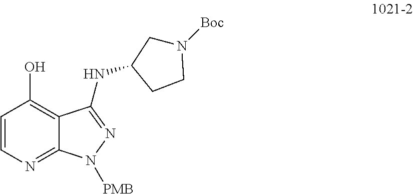

tert-Butyl (S)-3-((4-hydroxy-1-(4-methoxybenzyl)-1H-pyrazolo[3,4-b]pyridin-3-yl)amino)pyrrolidine-1-carboxylate (1021-2) (400 mg, 32%) was obtained as a yellow oil from 3-bromo-4-chloro-1-(4-methoxybenzyl)-1H-pyrazolo[3,4-b]pyridine (1.0 g, 2.85 mmol) and tert-butyl (S)-3-aminopyrrolidine-1-carboxylate (531 mg, 2.85 mmol) following a procedure adapted from that for 934, Method 3.1.2. LC-MS (ESI): m/z (M+1) 440.52.

### 3.17.2 Preparation of tert-butyl (S)-3-((1-(4-methoxybenzyl)-4-(((trifluoromethyl)sulfonyl)oxy)-1H-pyrazolo[3,4-b]pyridin-3-yl)amino)pyrrolidine-1-carboxylate (1021-3)

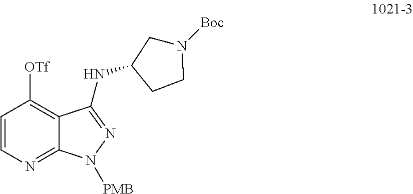

tert-Butyl (S)-3-((1-(4-methoxybenzyl)-4-(((trifluoromethyl)sulfonyl)oxy)-1H-pyrazolo[3,4-b]pyridin-3-yl)amino)pyrrolidine-1-carboxylate (1021-3) (550 mg, 96%) was obtained as a brown oil from tert-butyl (S)-3-((4-hydroxy-1-(4-methoxybenzyl)-1H-pyrazolo[3,4-b]pyridin-3-yl)amino)pyrrolidine-1-carboxylate (440 mg, 1.0 mmol) following a procedure adapted from that for 934, Method 3.1.3. LC-MS (ESI): m/z (M+1) 572.68.

### 3.17.3 Preparation of (S)-4-(tert-butyl)-N-(2-fluoro-4-(3-(pyrrolidin-3-ylamino)-1H-pyrazolo[3,4-b]pyridin-4-yl)benzyl)benzamide (1021-4)

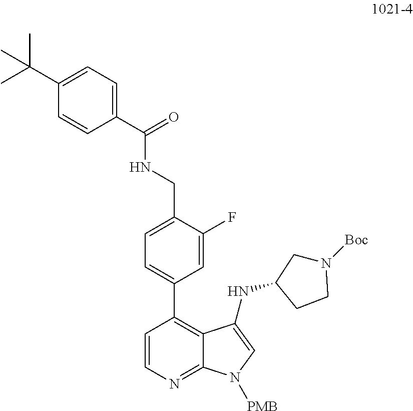

(S)-4-(tert-Butyl)-N-(2-fluoro-4-(3-(pyrrolidin-3-ylamino)-1H-pyrazolo[3,4-b]pyridin-4-yl)benzyl)benzamide (1021-4) (quant.) was obtained as a yellow oil from tert-butyl (S)-3-((1-(4-methoxybenzyl)-4-(((trifluoromethyl)sulfonyl)oxy)-1H-pyrazolo[3,4-b]pyridin-3-yl)amino)pyrrolidine-1-carboxylate (678 mg, 1.19 mmol) and 4-(tert-butyl)-N-(2-fluoro-4-(4,4,5,5-tetramethyl-1,3,2-dioxaborolan-2-yl)benzyl)benzamide (586 mg, 1.42 mmol) following a procedure adapted from that for 934, Method 3.1.4.

### 3.17.4 Preparation of (R)—N-(3-(3-((1-acryloylpyrrolidin-3-yl)amino)-1H-pyrazolo[3,4-b]pyridin-4-yl)-2-methylphenyl)-4-(tert-butyl)benzamide (1021)

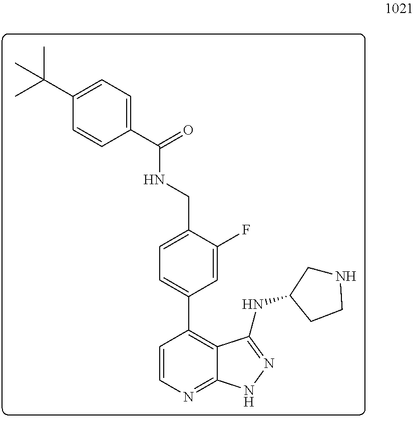 [SMILES: CC(C)(C)c1ccc(C(=O)NCc2ccc(-c3ccnc4[nH]nc(N[C@H]5CCNC5)c34)cc2F)cc1]

(S)-4-(tert-butyl)-N-(2-fluoro-4-(3-(pyrrolidin-3-ylamino)-1H-pyrazolo[3,4-b]pyridin-4-yl)benzyl)benzamide (1021) (45 mg, 8%) was obtained as a yellow solid from (S)-4-(tert-butyl)-N-(2-fluoro-4-(3-(pyrrolidin-3-ylamino)-1H-pyrazolo[3,4-b]pyridin-4-yl)benzyl) benzamide following a procedure adapted from that for Method 3.2.5. LC-MS (ESI): m/z (M+1) 487.68.

¹H NMR (400 MHz, DMSO) δ 12.57 (s, 1H), 9.11 (t, J=5.7 Hz, 1H), 8.43 (d, J=4.7 Hz, 1H), 8.34 (s, 1H), 7.87 (d, J=8.5 Hz, 2H), 7.60-7.39 (m, 5H), 6.99 (d, J=4.8 Hz, 1H), 4.60 (d, J=5.4 Hz, 3H), 4.14 (s, 1H), 3.20 (dd, J=11.4, 6.0 Hz, 1H), 3.10-2.93 (m, 3H), 2.08 (td, J=14.6, 7.4 Hz, 1H), 1.74-1.62 (m, 1H), 1.31 (s, 9H).

### Example 31: Synthesis of 5-tert-butyl-N-{[2-fluoro-4-(3-{[(3S)-pyrrolidin-3-yl]amino}-1H-pyrazolo[3,4-b]pyridin-4-yl)phenyl]methyl}-1,2,4-oxadiazole-3-carboxamide (Compound-50)

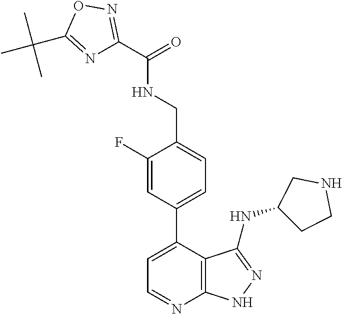 [SMILES: CC(C)(C)c1nc(C(=O)NCc2ccc(-c3ccnc4[nH]nc(N[C@H]5CCNC5)c34)cc2F)no1]

5-tert-butyl-N-{[2-fluoro-4-(3-{[(3S)-pyrrolidin-3-yl]amino}-1H-pyrazolo[3,4-b]pyridin-4-yl)phenyl]methyl}-1,2,4-oxadiazole-3-carboxamide was obtained from tert-butyl (S)-3-((4-(4-((5-(tert-butyl)-1,2,4-oxadiazole-3-carboxamido)methyl)-3-fluorophenyl)-1-(4-methoxybenzyl)-1H-pyrazolo[3,4-b]pyridin-3-yl)amino)pyrrolidine-1-carboxylate in accordance with Method C.

LC-MS (ESI): m/z (M+1) 479.2.

### Example 32: Synthesis of 4-tert-butyl-N-[2-fluoro-4-(3-{[(3R)-pyrrolidin-3-yl]amino}-1H-pyrazolo[3,4-b]pyridin-4-yl)phenyl]benzamide (Compound-42)

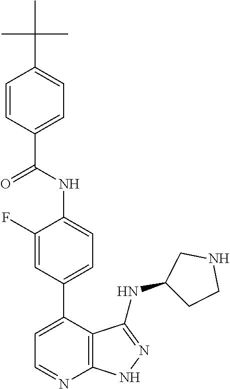 [SMILES: CC(C)(C)c1ccc(C(=O)Nc2ccc(-c3ccnc4[nH]nc(N[C@@H]5CCNC5)c34)cc2F)cc1]

### 3.18 Preparation of (R)-4-(tert-butyl)-N-(2-fluoro-4-(3-(pyrrolidin-3-ylamino)-1H-pyrazolo[3,4-b]pyridin-4-yl)phenyl)benzamide (1022)

### 3.18.1 Preparation of tert-butyl (R)-3-((4-(4-(4-(tert-butyl)benzamido)-3-fluorophenyl)-1-(4-methoxybenzyl)-1H-pyrazolo[3,4-b]pyridin-3-yl)amino)pyrrolidine-1-carboxylate (1022-2)

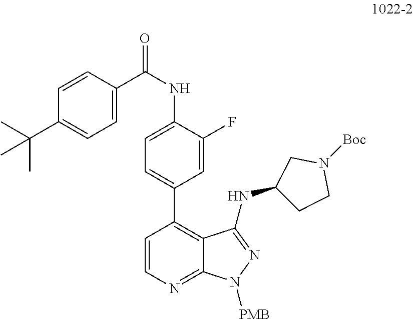

tert-Butyl (R)-3-((4-(4-(4-(tert-butyl)benzamido)-3-fluorophenyl)-1-(4-methoxybenzyl)-1H-pyrazolo[3,4-b]pyridin-3-yl)amino)pyrrolidine-1-carboxylate (1022-2) (quant.) was obtained as a yellow oil from tert-butyl (R)-3-((1-(4-methoxybenzyl)-4-(((trifluoromethyl)sulfonyl)oxy)-1H-pyrazolo[3,4-b]pyridin-3-yl)amino)pyrrolidine-1-carboxylate (150 mg, 0.26 mmol) and 4-(tert-butyl)-N-(2-fluoro-4-(4,4,5,5-tetramethyl-1,3,2-dioxaborolan-2-yl)phenyl)benzamide (125 mg, 0.31 mmol) following a procedure adapted from that for 934, Method 3.1.4.

### Preparation of (R)-4-(tert-butyl)-N-(2-fluoro-4-(3-(pyrrolidin-3-ylamino)-1H-pyrazolo[3,4-b]pyridin-4-yl)phenyl)benzamide (1022)

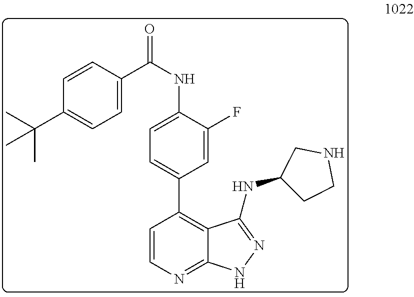 [SMILES: CC(C)(C)c1ccc(C(=O)Nc2ccc(-c3ccnc4[nH]nc(N[C@@H]5CCNC5)c34)cc2F)cc1]

(R)-4-(tert-Butyl)-N-(2-fluoro-4-(3-(pyrrolidin-3-ylamino)-1H-pyrazolo[3,4-b]pyridin-4-yl)phenyl)benzamide (1022) (40 mg, 32%) was obtained as a white solid from tert-butyl (R)-3-((4-(4-(4-(tert-butyl)benzamido)-3-fluorophenyl)-1-(4-methoxybenzyl)-1H-pyrazolo [3,4-b]pyridin-3-yl)amino)pyrrolidine-1-carboxylate following a procedure adapted from Method 3.1.5. LC-MS (ESI): m/z (M+1) 473.58. ¹H NMR (400 MHz, DMSO) δ 12.63-12.53 (m, 1H), 10.21 (s, 1H), 8.45 (d, J=4.8 Hz, 1H), 8.36 (s, 1H), 7.96 (d, J=8.3 Hz, 2H), 7.88 (t, J=8.3 Hz, 1H), 7.66 (d, J=11.7 Hz, 1H), 7.62-7.50 (m, 3H), 7.04 (d, J=4.6 Hz, 1H), 4.61 (d, J=5.5 Hz, 1H), 4.12 (s, 1H), 3.13-2.91 (m, 4H), 2.12-2.05 (m, 1H), 1.71-1.63 (m, 1H), 1.34 (s, 9H).

### Example 33: Synthesis of 4-tert-butyl-N-[2-fluoro-4-(3-{[(3R)-pyrrolidin-3-yl]amino}-1H-pyrazolo[3,4-b]pyridin-4-yl)phenyl]benzamide (Compound-45)

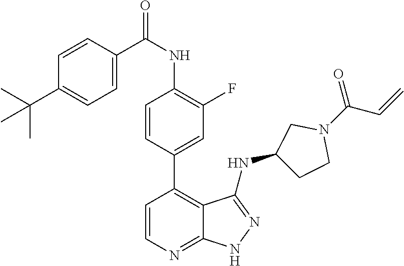 [SMILES: C=CC(=O)N1CC[C@@H](Nc2n[nH]c3nccc(-c4ccc(NC(=O)c5ccc(C(C)(C)C)cc5)c(F)c4)c23)C1]

4-tert-butyl-N-[2-fluoro-4-(3-{[(3R)-pyrrolidin-3-yl]amino}-1H-pyrazolo[3,4-b]pyridin-4-yl)phenyl]benzamide was obtained from (R)-4-(tert-butyl)-N-(2-fluoro-4-(3-(pyrrolidin-3-ylamino)-1H-pyrazolo[3,4-b]pyridin-4-yl)phenyl)benzamide in accordance with Method E. LC-MS (ESI): m/z (M+1) 527.2.

### Example 34A: (R)—N-(4-(3-(3-aminopyrrolidin-1-yl)-1H-pyrazolo[3,4-b]pyridin-4-yl)-2-fluorobenzyl)-5-(2-cyanopropan-2-yl)isoxazole-3-carboxamide

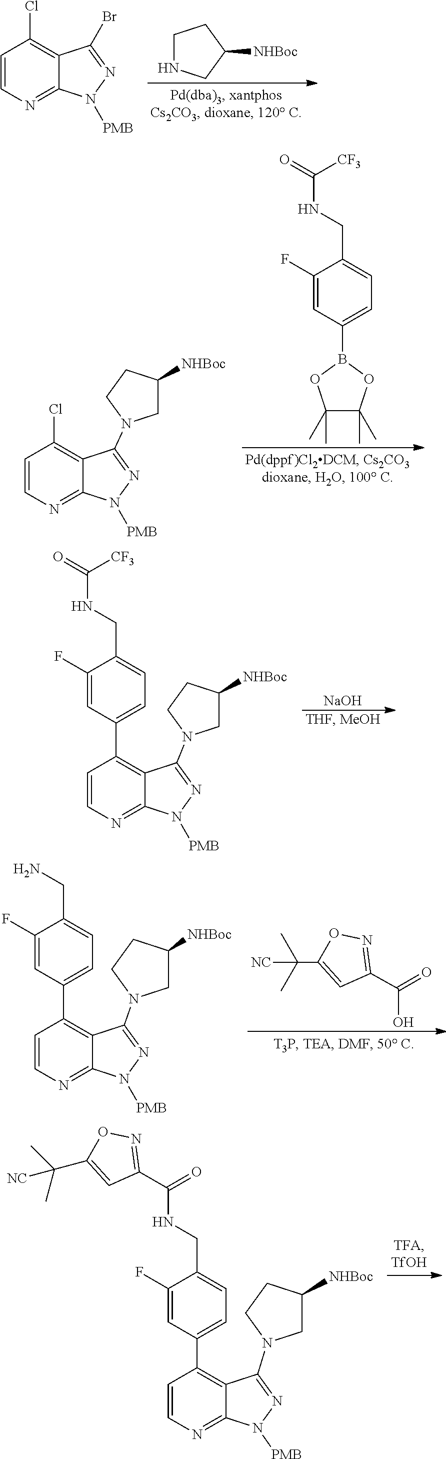

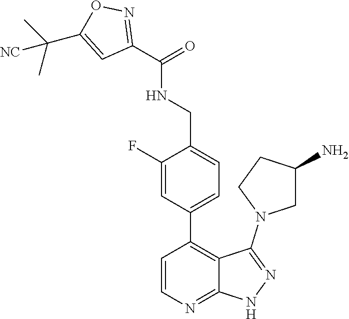 [SMILES: CC(C)(C#N)c1cc(C(=O)NCc2ccc(-c3ccnc4[nH]nc(N5CC[C@@H](N)C5)c34)cc2F)no1]

At 120° C. under N₂ atmosphere, to a stirred solution of 3-bromo-4-chloro-1-(4-methoxybenzyl)-1H-pyrazolo[3,4-b]pyridine (120 g, 342 mmol) in dioxane (3.6 L) were added tert-butyl (R)-pyrrolidin-3-ylcarbamate (63.7 g, 342 mmol), Pd₂(dba)₃ (15.6 g, 17.1 mmol), xantphos (29.6 g, 51.3 mmol) and CS₂CO₃ (222 g, 684 mmol). After being stirred at 120° C. for 8 hr, the reaction mixture was cooled down to room temperature and filtered through a pad of celite. The filtration was quenched with H₂O and extracted with ethyl acetate (3×). The combined organic layers were washed with brine and dried over Na₂SO₄. Solvents were removed and the residue was purified by flash chromatography (silica gel, 0˜50% ethyl acetate in petroleum ether) to provide tert-butyl (R)-(1-(4-chloro-1-(4-methoxybenzyl)-1H-pyrazolo[3,4-b]pyridin-3-yl)pyrrolidin-3-yl)carbamate (60 g, 39%) as a yellow solid. LCMS (ESI) m/z (M/M+2) 458.25/460.22.

At 100° C. under N₂ atmosphere, to a stirred solution of tert-butyl (R)-(1-(4-chloro-1-(4-methoxybenzyl)-1H-pyrazolo[3,4-b]pyridin-3-yl)pyrrolidin-3-yl)carbamate (2.0 g, 4.37 mmol) in dioxane/H₂O (15 mL/8 mL) were added 2,2,2-trifluoro-N-(2-fluoro-4-(4,4,5,5-tetramethyl-1,3,2-dioxaborolan-2-yl)benzyl)acetamide (1.97 g, 5.68 mmol), Pd(dppf)Cl₂.DCM (541 mg, 0.66 mmol), and Cs₂CO₃(4.27 g, 13.1 mmol). After being stirred at 100° C. overnight, the reaction mixture was cooled down to room temperature and filtered through a pad of celite. The filtration was quenched with H₂O and extracted with ethyl acetate (3×). The combined organic layers were washed with brine and dried over Na₂SO₄. Solvents were removed and the residue was purified by flash chromatography (silica gel, 0˜50% ethyl acetate in petroleum ether) to provide tert-butyl (R)-(1-(4-(4-(aminomethyl)-3-fluorophenyl)-1-(4-methoxybenzyl)-1H-pyrazolo[3,4-b]pyridin-3-yl)pyrrolidin-3-yl)carbamate (2.0 g, 84%) as a yellow solid. LCMS (ESI) m/z (M+1): 547.31

At 50° C., to a stirred solution of tert-butyl (R)-(1-(4-(4-(aminomethyl)-3-fluorophenyl)-1-(4-methoxybenzyl)-1H-pyrazolo[3,4-b]pyridin-3-yl)pyrrolidin-3-yl)carbamate (218 mg, 0.4 mmol) in DMF (3 mL) were added 5-(2-cyanopropan-2-yl)isoxazole-3-carboxylic acid (86 mg, 0.48 mmol), T3P (763 mg, 1.2 mmol, 50% in ethyl acetate) and TEA (0.28 mL, 2.0 mmol). After being stirred at room temperature overnight, the reaction mixture was quenched with sat. NaHCO₃ and extracted with ethyl acetate (3×). The combined organic layers were wash with brine and dried over Na₂SO₄. Solvents were removed under vacuum to provide tert-butyl (R)-(1-(4-(4-((5-(2-cyanopropan-2-yl)isoxazole-3-carboxamido)methyl)-3-fluorophenyl)-1-(4-methoxybenzyl)-1H-pyrazolo[3,4-b]pyridin-3-yl)pyrrolidin-3-yl)carbamate as a yellow solid which was used in the next step without purification.

At 0° C., to a stirred solution of tert-butyl (R)-(1-(4-(4-((5-(2-cyanopropan-2-yl)isoxazole-3-carboxamido)methyl)-3-fluorophenyl)-1-(4-methoxybenzyl)-1H-pyrazolo[3,4-b]pyridin-3-yl)pyrrolidin-3-yl)carbamate in TFA (3 mL) was added TfOH (0.5 mL). After being stirred at room temperature for 4 hr, the reaction mixture was concentrated. The residue was quenched with saturated NaHCO₃ and extracted with DCM (3×). The combined organic layers were washed with brine and dried over Na₂SO₄. Solvents were removed under vacuum and the crude product was purified by prep. HPLC (C18, 0˜90% acetonitrile in H₂O with 0.1% formic acid) to provide (R)—N-(4-(3-(3-aminopyrrolidin-1-yl)-1H-pyrazolo[3,4-b]pyridin-4-yl)-2-fluorobenzyl)-5-(2-cyanopropan-2-yl)isoxazole-3-carboxamide (200 mg, 75% over 2 steps) as a white solid. LCMS (ESI) m/z (M+1): 489.21. ¹H NMR (400 MHz, DMSO) δ 12.89 (s, 1H), 9.53 (dd, J=50.1, 44.3 Hz, 1H), 8.44 (t, J=17.2 Hz, 1H), 8.29 (d, J=36.8 Hz, 1H), 7.67-7.34 (m, 3H), 7.09 (d, J=4.7 Hz, 1H), 7.03 (s, 1H), 4.59 (d, J=5.7 Hz, 2H), 3.53-3.42 (m, 1H), 3.12 (dd, J=10.4, 6.8 Hz, 1H), 2.90-2.82 (m, 1H), 2.80-2.67 (m, 2H), 1.92 (td, J=14.5, 7.5 Hz, 1H), 1.79 (s, 6H), 1.52 (dt, J=12.8, 5.7 Hz, 1H).

### Example 34B: (R)-3-(tert-butyl)-N-(2-fluoro-4-(3-(3-(methylamino)pyrrolidin-1-yl)-1H-pyrazolo[3,4-b]pyridin-4-yl)benzyl)-N-methyl-1,2,4-oxadiazole-5-carboxamide

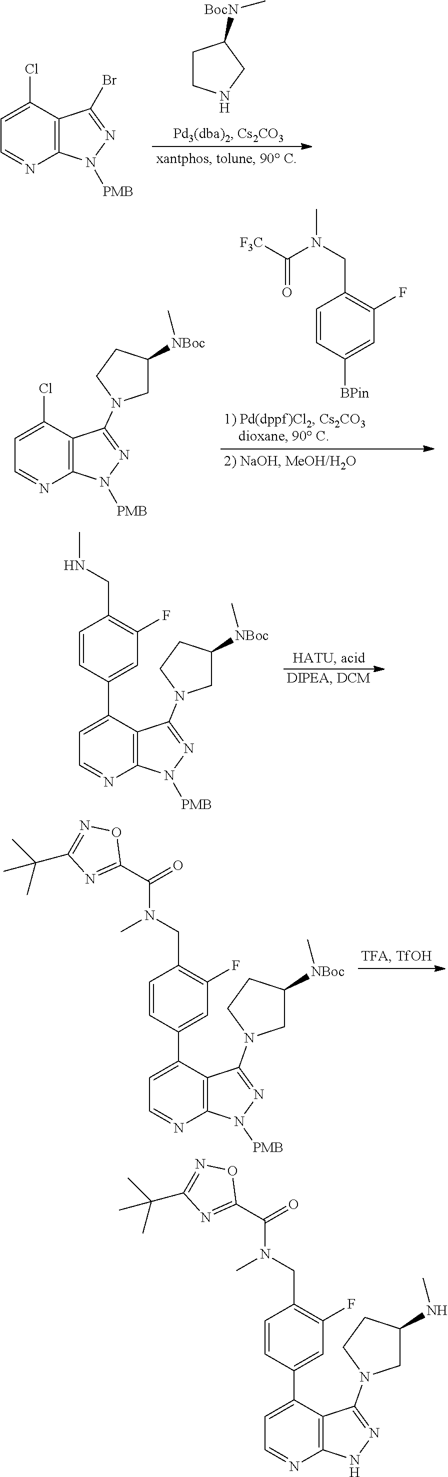

At 90° C., to a stirred solution of 3-bromo-4-chloro-1-(4-methoxybenzyl)-1H-pyrazolo[3,4-b]pyridine (8 g, 22.79 mmol) in dioxane (120 mL) were added tert-butyl (R)-methyl(pyrrolidin-3-yl)carbamate (5.01 g, 25.07 mmol), Pd₂(dba)₃ (1.05 g, 1.14 mmol), xantphos (1.32 g, 2.28 mmol) and Cs₂CO₃ (29.7 g, 91.16 mmol) under nitrogen atmosphere. After being stirred at 90° C. for 8 hours, the reaction mixture was cooled down to room temperature and filtered through a pad of celite. The filtration was quenched with water and extracted with EtOAc (3×40 ml). The combined organic layers were washed with brine and dried over Na₂SO₄. Solvents were removed and the residue was purified by flash chromatography (silica gel, 0˜50% ethyl acetate in petroleum ether) to provide tert-butyl (R)-(1-(4-chloro-1-(4-methoxy benzyl)-1H-pyrazolo[3,4-b]pyridin-3-yl)pyrrolidin-3-yl)(methyl)carbamate (3.0 g, 28%) as a yellow oil. LCMS (ESI) m/z (M+1): 472.27.

At 85° C., to a stirred solution of tert-butyl (R)-(1-(4-chloro-1-(4-methoxy benzyl)-1H-pyrazolo [3,4-b]pyridin-3-yl)pyrrolidin-3-yl)(methyl)carbamate (1.5 g, 3.18 mmol) in dioxane/water (15 ml/8 ml) were added 2,2,2-trifluoro-N-(2-fluoro-4-(4,4,5,5-tetramethyl-1,3,2-dioxaborolan-2-yl)benzyl)-N-methylacetamide (5.01 g, (2.30 g, 4.77 mmol), Pd(dppf)Cl₂.DCM (200 mg, 0.16 mmol) and Cs₂CO₃ (3.1 g, 9.54 mmol) under nitrogen atmosphere. After being stirred at 85° C. overnight, the reaction mixture was cooled down to room temperature and filtered through a pad of celite. The filtration was quenched with water and extracted with EtOAc (3×20 ml). The combined organic layers were washed with brine and dried over Na₂SO₄. Solvents were removed and the residue was dissolved in MeOH (3 ml) and water (1 ml) which was treated with 5N NaOH (1 ml). The mixture was stirred for another 4 hours and then concentrated to afford the residue which was purified by flash chromatography (silica gel, 0˜50% ethyl acetate in petroleum ether) to provide tert-butyl (R)-(1-(4-(3-fluoro-4-((methylamino)methyl) phenyl)-1-(4-methoxybenzyl)-1H-pyrazolo[3,4-b]pyridin-3-yl)pyrrolidin-3-yl)(methyl)carbamate (1.12 g, 61%) as a yellow solid. LCMS (ESI) m/z (M+1): 575.44.

At 50° C., to a stirred solution of tert-butyl (R)-(1-(4-(3-fluoro-4-((methylamino)methyl)phenyl)-1-(4-methoxybenzyl)-1H-pyrazolo[3,4-b]pyridin-3-yl)pyrrolidin-3-yl)(methyl)carbamate (1.0 g, 1.74 mmol) in DMF (3 ml) were added 5-(2-cyanopropan-2-yl)isoxazole-3-carboxylic acid (355 mg, 2.09 mmol), HATU (993 mg, 2.61 mmol) and DIEA (0.92 ml, 523 mmol). After being stirred at room temperature overnight, the reaction mixture was quenched with sat.aq.NaHCO₃ and extracted with EtOAc (3×10 ml). The combined organic layers were washed with brine and dried over Na₂SO₄. Solvents were removed under vacuum and the residue was purified by flash chromatography (silica gel, 0˜50% ethyl acetate in petroleum ether) to provide tert-butyl (R)-(1-(4-(4-((3-(tert-butyl)-N-methyl-1,2,4-oxadiazole-5-carboxamido)methyl)-3-fluorophenyl)-1-(4-methoxybenzyl)-1H-pyrazolo[3,4-b]pyridin-3-yl)pyrrolidin-3-yl)(methyl)carbamate (850 mg, 67%) as a yellow solid. LCMS (ESI) m/z (M-55):673.44.

At 0° C., to a stirred solution of tert-butyl(R)-(1-(4-(4-((3-(tert-butyl)-N-methyl-1,2,4-oxadiazole-5-carboxamido)methyl)-3-fluorophenyl)-1-(4-methoxybenzyl)-1H-pyrazolo[3,4-b]pyridin-3-yl)pyrrolidin-3-yl)(methyl)carbamate (150 mg, 206 mmol) in TFA (1.5 ml) was added TfOH (0.5 ml). After being stirred at r.t. for 1 hr, the reaction mixture was concentrated to afford the residue which was quenched with sat.aq.NaHCO₃ and extracted with DCM (3×5 ml). The combined organic layers were washed with brine and dried over Na₂SO₄. Solvents were removed under vacuum and the crude product was purified by prep. HPLC (C18, 0˜90% acetonitrile in H₂O with 0.1% formic acid) to provide (R)-3-(tert-butyl)-N-(2-fluoro-4-(3-(3-(methylamino)pyrrolidin-1-yl)-1H-pyrazolo[3,4-b]pyridin-4-yl)benzyl)-N-methyl-1,2,4-oxadiazole-5-carboxamide (34 mg, 32%) as a white solid. LCMS (ESI) m/z (M+1): 507.27, 1H NMR (400 MHz, DMSO) δ 13.02 (s, 1H), 8.77 (d, J=22.3 Hz, 2H), 8.51 (dd, J=4.7, 3.1 Hz, 1H), 7.65-7.48 (m, 3H), 7.14 (dd, J=10.2, 4.7 Hz, 1H), 4.92 (d, J=44.8 Hz, 2H), 3.44-3.35 (m, 1H), 3.24-3.06 (m, 4H), 2.74-2.51 (m, 5H), 1.99-1.91 (m, 1H), 1.69-1.60 (m, 1H), 1.34 (d, J=19.8 Hz, 9H).

The following compounds were prepared by analogous methods:

| Chemical structure | Chemical names | LC-MS/HNMR |
|---|---|---|
| 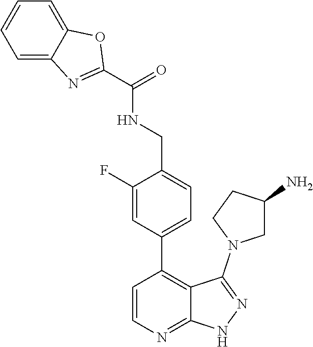 [SMILES: N[C@@H]1CCN(c2n[nH]c3nccc(-c4ccc(CNC(=O)c5nc6ccccc6o5)c(F)c4)c23)C1] | (R)-N-(4-(3-(3- aminopyrrolidin-1-yl)- 1H- pyrazolo[3,4-b]pyridin- 4-yl)-2-fluorobenz- yl)benzo[d]oxazole-2- carboxamide | LCMS (ESI) m/z (M + 1): 472.25 ¹H NMR (400 MHz, DMSO) δ 12.97 (s, 1H), 10.12-9.77 (m, 1H), 8.49 (d, J = 4.7 Hz, 1H), 8.01 (s, 3H), 7.91 (dd, J = 17.8, 7.8 Hz, 2H), 7.62-7.48 (m, 5H), 7.12 (d, J = 4.7 Hz, 1H), 4.64 (d, J = 6.0 Hz, 2H), 3.65 (s, 1H), 3.26 (dd, J = 10.8, 7.2 Hz, 1H), 2.95 (dd, J = 11.0, 5.4 Hz, 1H), 2.83 (dd, J = 16.8, 7.4 Hz, 1H), 2.73-2.65 (m, 1H), 2.00 (dt, J = 15.1, 7.3 Hz, 1H), 1.71-1.58 (m, 1H) |
| 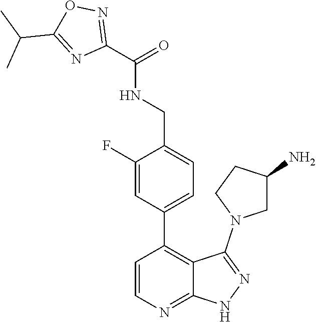 [SMILES: CC(C)c1nc(C(=O)NCc2ccc(-c3ccnc4[nH]nc(N5CC[C@@H](N)C5)c34)cc2F)no1] | (R)-N-(4-(3-(3- aminopyrrolidin-1-yl)- 1H- pyrazolo[3,4-b]pyridin- 4-yl)-2-fluorobenz- yl)-5-isopropyl-1,2,4- oxadiazole-3- carboxamide | LCMS (ESI) m/z (M + 1): 465.20 ¹H NMR (400 MHz, DMSO) δ 12.97 (s, 1H), 9.58 (t, J = 6.0 Hz, 1H), 8.49 (d, J = 4.7 Hz, 1H), 8.02 (s, 3H), 7.62-7.44 (m, 2H), 7.12 (d, J = 4.7 Hz, 1H), 4.59 (d, J = 6.0 Hz, 2H), 3.68-3.61 (m, 1H), 3.43-3.34 (m, 1H), 3.29- 3.21 (m, 1H), 2.96 (dd, J = 10.9, 5.5 Hz, 1H), 2.79 (d, J = 7.4 Hz, 1H), 2.74-2.62 (m, 1H), 1.98 (dd, J = 13.4, 7.7 Hz, 1H), 1.73-1.61 (m, 1H), 1.37 (d, J = 7.0 Hz, 6H). |
| 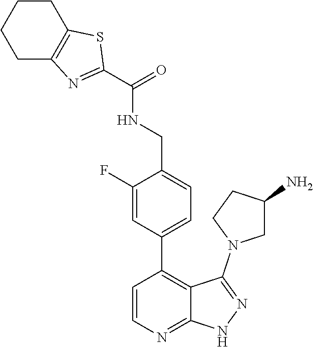 [SMILES: N[C@@H]1CCN(c2n[nH]c3nccc(-c4ccc(CNC(=O)c5nc6c(s5)CCCC6)c(F)c4)c23)C1] | (R)-N-(4-(3-(3- aminopyrrolidin-1-yl)- 1H- pyrazolo[3,4-b]pyridin- 4-yl)-2-fluorobenzyl)- 4,5,6,7-tetrahydro- benzo[d]thiazole-2- carboxamide | LCMS (ESI) m/z (M + 1): 492.28 ¹H NMR (400 MHz, DMSO) δ 12.97 (s, 1H), 9.45-9.24 (m, 1H), 8.49 (d, J = 4.7 Hz, 1H), 8.09 (s, 3H), 7.51 (dd, J = 12.9, 7.3 Hz, 2H), 7.11 (d, J = 4.7 Hz, 1H), 4.57 (d, J = 6.1 Hz, 2H), 3.75-3.52 (m, 1H), 3.41-3.24 (m, 1H), 2.99 (dd, J = 11.0, 5.4 Hz, 1H), 2.88-2.75 (m, 3H), 2.70-2.61 (m, 1H), 2.57-2.50 (m, J = 1.6 Hz, 3H), 1.97 (dd, J = 13.4, 7.7 Hz, 1H), 1.89-1.77 (m, 3H), 1.73-1.57 (m, 1H) |
| 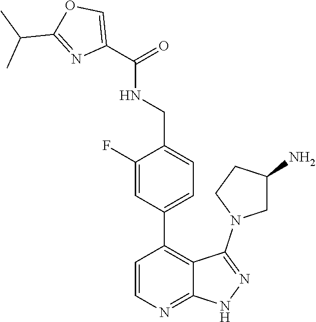 [SMILES: CC(C)c1nc(C(=O)NCc2ccc(-c3ccnc4[nH]nc(N5CC[C@@H](N)C5)c34)cc2F)co1] | (R)-N-(4-(3-(3- aminopyrrolidin-1-yl)- 1H- pyrazolo[3,4-b]pyridin- 4-yl)-2-fluorobenzyl)- 2-isopropyloxazole-4- carboxamide | LCMS (ESI) m/z (M + 1): 464.22 ¹H NMR (400 MHz, DMSO) δ 12.99 (s, 1H), 8.80 (t, J = 6.1 Hz, 1H), 8.57 (s, 1H), 8.49 (d, J = 4.8 Hz, 1H), 8.17 (s, 3H), 7.52-7.41 (m, 3H), 7.11 (d, J = 4.8 Hz, 1H), 4.55 (d, J = 6.0 Hz, 2H), 3.63 (s, 1H), 3.28 (dd, J = 10.8, 7.2 Hz, 1H), 3.20-3.09 (m, 1H), 2.98 (dd, J = 10.9, 5.5 Hz, 1H), 2.81 (dd, J = 16.6, 7.3 Hz, 1H), 2.67 (dd, J = 14.0, 8.5 Hz, 1H), 1.98 (dd, J = 13.2, 7.7 Hz, 1H), 1.66 (dd, J = 12.9, 7.5 Hz, 1H), 1.31 (d, J = 7.0 Hz, 6H). |
| 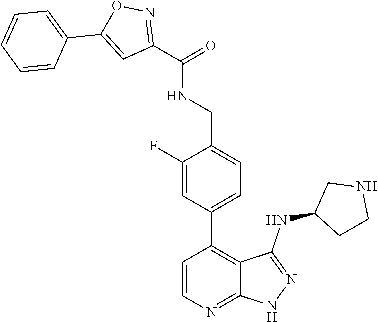 [SMILES: O=C(NCc1ccc(-c2ccnc3[nH]nc(N[C@@H]4CCNC4)c23)cc1F)c1cc(-c2ccccc2)on1] | (R)-N-(2-fluoro-4-(3- (pyrrolidin-3-ylamino)- 1H- pyrazolo[3,4-b]pyridin- 4-yl)benzyl)-5- phenylisoxazole-3- carboxamide | LCMS (ESI) m/z (M + 1): 498.19 ¹H NMR (400 MHz, DMSO) δ 12.71 (s, 1H), 9.50 (t, J = 6.0 Hz, 1H), 9.22 (s, 2H), 8.47 (d, J = 4.8 Hz, 1H), 8.25-7.78 (m, 2H), 7.64-7.53 (m, 5H), 7.45 (s, 1H), 7.05 (d, J = 4.8 Hz, 1H), 4.62 (d, J = 5.8 Hz, 2H), 4.23 (s, 1H), 3.36 (dd, J = 13.0, 7.0 Hz, 1H), 3.31-3.10 (m, 3H), 2.23-2.10 (m, 1H), 2.00-1.75 (m, 1H). |
| 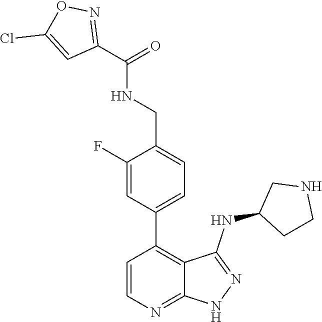 [SMILES: O=C(NCc1ccc(-c2ccnc3[nH]nc(N[C@@H]4CCNC4)c23)cc1F)c1cc(Cl)on1] | (R)-5-chloro-N-(2- fluoro-4-(3- (pyrrolidin-3- ylamino)-1H- pyrazolo[3,4-b]pyridin- 4-yl)benzyl)isoxazole- 3-carboxamide | LCMS (ESI) m/z (M + 1): 456.14 ¹H NMR (400 MHz, DMSO) δ 12.72 (s, 1H), 9.58 (t, J = 5.8 Hz, 1H), 9.23 (s, 2H), 8.47 (d, J = 4.8 Hz, 1H), 7.65-7.41 (m, 3H), 7.16 (s, 1H), 7.05 (d, J = 4.8 Hz, 1H), 4.58 (d, J = 5.9 Hz, 2H), 4.28-4.16 (m, 1H), 3.42-3.30 (m, 1H), 3.29-3.13 (m, 3H), 2.24-2.10 (m, 1H), 1.90-1.77 (m, 1H) |
| 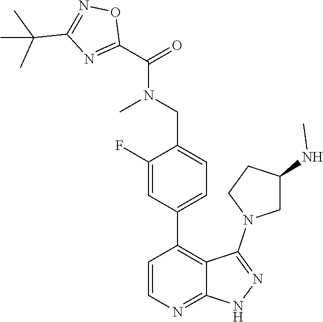 [SMILES: CN[C@@H]1CCN(c2n[nH]c3nccc(-c4ccc(CN(C)C(=O)c5nc(C(C)(C)C)no5)c(F)c4)c23)C1] | (R)-3-(tert-butyl)-N-(2- fluoro-4-(3-(3-(methyl- amino)pyrrolidin-1-yl)- 1H- pyrazolo[3,4-b]pyridin- 4-yl)benzyl)-N-methyl- 1,2,4-oxadiazole-5- carboxamide | LCMS (ESI) m/z (M + 1): 507.27 ¹H NMR (400 MHz, DMSO) δ 13.02 (s, 1H), 8.77 (d, J = 22.3 Hz, 2H), 8.51 (dd, J = 4.7, 3.1 Hz, 1H), 7.65-7.48 (m, 3H), 7.14 (dd, J = 10.2, 4.7 Hz, 1H), 4.92 (d, J = 44.8 Hz, 2H), 3.44-3.35 (m, 1H), 3.24-3.06 (m, 4H), 2.74- 2.51 (m, 5H), 1.99-1.91 (m, 1H), 1.69-1.60 (m, 1H), 1.34 (d, J = 19.8 Hz, 9H) |
| 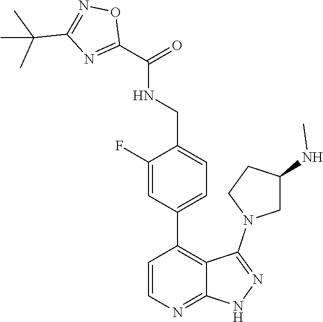 [SMILES: CN[C@@H]1CCN(c2n[nH]c3nccc(-c4ccc(CNC(=O)c5nc(C(C)(C)C)no5)c(F)c4)c23)C1] | (R)-3-(tert-butyl)-N-(2- fluoro-4-(3-(3- (methylamino)pyrrolidin- 1-yl)-1H- pyrazolo[3,4-b]pyridin- 4-yl)benzyl)-1,2,4- oxadiazole-5- carboxamide | LCMS (ESI) m/z (M + 1): 493.25 ¹H NMR (400 MHz, DMSO) δ 13.00 (s, 1H), 9.97 (t, J = 6.0 Hz, 1H), 8.85 (s, 2H), 8.50 (d, J = 4.7 Hz, 1H), 7.65-7.42 (m, 3H), 7.13 (d, J = 4.7 Hz, 1H), 4.59 (d, J = 5.9 Hz, 2H), 3.61 (s, 1H), 3.28 (dd, J = 11.0, 7.3 Hz, 1H), 3.05 (dd, J = 11.1, 5.6 Hz, 1H), 2.76 (dd, J = 15.5, 7.8 Hz, 1H), 2.64 (dd, J = 16.5, 7.5 Hz, 1H), 2.03-1.94 (m, 1H), 1.75-1.66 (m, 1H), 1.37 (s, 9H) |
| 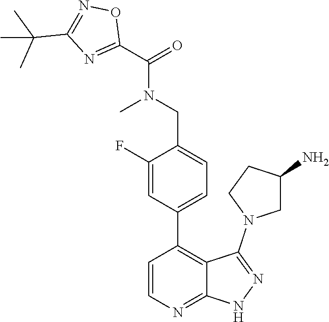 [SMILES: CN(Cc1ccc(-c2ccnc3[nH]nc(N4CC[C@@H](N)C4)c23)cc1F)C(=O)c1nc(C(C)(C)C)no1] | (R)-N-(4-(3-(3- aminopyrrolidin-1-yl)- 1H- pyrazolo[3,4-b]pyridin- 4-yl)-2-fluorobenzyl)- 3-(tert-butyl)-N-methyl- 1,2,4-oxadiazole-5- carboxamide | LCMS (ESI) m/z (M + 1): 493.25 ¹H NMR (400 MHz, DMSO) δ 13.00 (s, 1H), 8.57-8.45 (m, 1H), 8.12 (d, J = 10.6 Hz, 3H), 7.71-7.43 (m, 3H), 7.14 (dd, J = 11.7, 4.7 Hz, 1H), 4.92 (d, J = 41.4 Hz, 2H), 3.65 (s, 1H), 3.39-3.26 (m, 1H), 3.25-2.97 (m, 4H), 2.87- 2.61 (m, 2H), 2.03-1.89 (m, 1H), 1.69-1.57 (m, 1H), 1.34 (d, J = 19.5 Hz, 9H) |
| 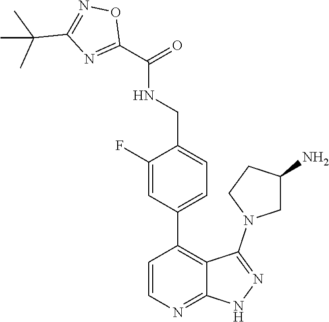 [SMILES: CC(C)(C)c1noc(C(=O)NCc2ccc(-c3ccnc4[nH]nc(N5CC[C@@H](N)C5)c34)cc2F)n1] | (R)-N-(4-(3-(3- aminopyrrolidin-1-yl)- 1H- pyrazolo[3,4-b]pyridin- 4-yl)-2-fluorobenzyl)-3- (tert-butyl)-1,2,4- oxadiazole-5- carboxamide | LCMS (ESI) m/z (M + 1): 478.26 ¹H NMR (400 MHz, DMSO) δ 12.97 (s, 1H), 9.95 (t, J = 5.9 Hz, 1H), 8.50 (d, J = 4.7 Hz, 1H), 8.03 (s, 3H), 7.55 (dt, J = 13.5, 7.9 Hz, 3H), 7.12 (d, J = 4.7 Hz, 1H), 4.60 (d, J = 5.7 Hz, 2H), 3.64 (s, 1H), 3.26 (dd, J = 10.7, 7.3 Hz, 1H), 2.95 (dd, J = 10.7, 5.4 Hz, 1H), 2.83 (dd, J = 16.9, 7.5 Hz, 1H), 2.68 (dd, J = 15.1, 7.6 Hz, 1H), 1.99 (dd, J = 13.1, 8.1 Hz, 1H), 1.65 (d, J = 5.4 Hz, 1H), 1.37 (s, 9H). |
| 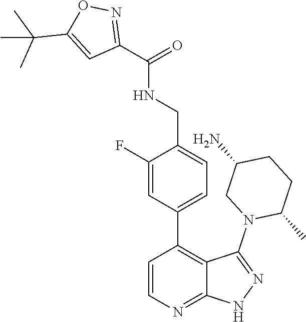 [SMILES: C[C@H]1CC[C@@H](N)CN1c1n[nH]c2nccc(-c3ccc(CNC(=O)c4cc(C(C)(C)C)on4)c(F)c3)c12] | N-(4-(3-((2S,5R)-5- amino-2- methylpiperidin-1-yl)- 1H- pyrazolo[3,4-b]pyridin- 4-yl)-2-fluorobenzyl)-5- (tert-butyl)isoxazole-3- carboxamide | LCMS (ESI) m/z (M + 1): 506.30 ¹H NMR (400 MHz, DMSO) δ 13.07 (s, 1H), 9.36 (t, J = 6.0 Hz, 1H), 8.50 (d, J = 4.7 Hz, 1H), 8.24 (s, 3H), 7.59-7.46 (m, 3H), 7.12 (d, J = 4.8 Hz, 1H), 6.59 (s, 1H), 4.56 (d, J = 6.0 Hz, 2H), 3.46 (d, J = 7.7 Hz, 1H), 3.22-3.08 (m, 2H), 2.86-2.75 (m, 1H), 1.72 (d, J = 12.0 Hz, 1H), 1.66-1.53 (m, 1H), 1.33 (s, 9H), 1.26-1.20 (m, 1H), 1.08 (d, J = 13.1 Hz, 1H), 0.63 (d, J = 6.8 Hz, 3H). |
| 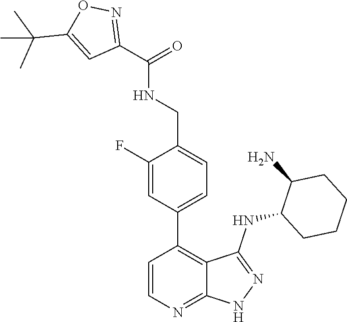 [SMILES: CC(C)(C)c1cc(C(=O)NCc2ccc(-c3ccnc4[nH]nc(N[C@H]5CCCC[C@@H]5N)c34)cc2F)no1] | N-(4-(3-(((1S,2S)-2- aminocyclohexyl)amino)- 1H- pyrazolo[3,4-b]pyridin- 4-yl)-2-fluorobenzyl)- 5-(tert-butyl)isoxazole- 3-carboxamide | LCMS (ESI) m/z (M + 1): 506.31 ¹H NMR (400 MHz, DMSO) δ 12.58 (s, 1H), 9.35 (t, J = 5.9 Hz, 1H), 8.45 (d, J = 4.8 Hz, 1H), 7.89 (s, 3H), 7.61 (d, J = 11.2 Hz, 1H), 7.53 (d, J = 3.9 Hz, 2H), 6.98 (d, J = 4.8 Hz, 1H), 6.62 (s, 1H), 4.58 (d, J = 5.9 Hz, 2H), 4.30 (d, J = 7.8 Hz, 1H), 3.67 (d, J = 4.7 Hz, 1H), 3.07 (s, 1H), 2.06-1.92 (m, 3H), 1.72- 1.60 (m, 2H), 1.47-1.25 (m, 12H) |
| 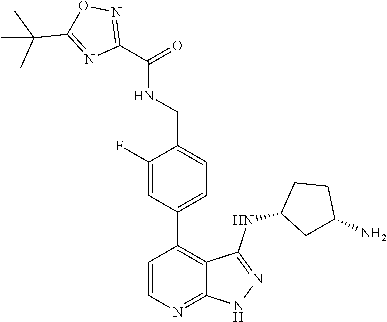 [SMILES: CC(C)(C)c1nc(C(=O)NCc2ccc(-c3ccnc4[nH]nc(N[C@@H]5CC[C@H](N)C5)c34)cc2F)no1] | N-(4-(3-(((1R,3S)-3- aminocyclopentyl)ami- no)-1H- pyrazolo[3,4-b]pyridin- 4-yl)-2-fluorobenzyl)- 5-(tert-butyl)-1,2,4- oxadiazole-3- carboxamide | LCMS (ESI) m/z (M + 1): 493.32 ¹H NMR (400 MHz, DMSO) δ 12.55 (s, 1H), 9.57 (t, J = 5.9 Hz, 1H), 8.44 (d, J = 4.8 Hz, 1H), 7.91 (s, 3H), 7.61-7.43 (m, 3H), 6.99 (d, J = 4.8 Hz, 1H), 4.60 (d, J = 5.9 Hz, 2H), 4.37 (s, 1H), 3.88 (t, J = 7.2 Hz, 1H), 3.48 (d, J = 6.1 Hz, 1H), 2.48-2.39 (m, 2H), 2.01-1.87 (m, 2H), 1.71-1.64 (m, 1H), 1.61-1.52 (m, 1H), 1.44 (s, 9H) |
| 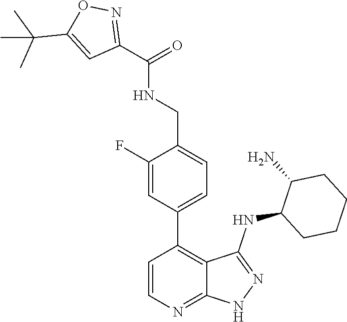 [SMILES: CC(C)(C)c1cc(C(=O)NCc2ccc(-c3ccnc4[nH]nc(N[C@@H]5CCCC[C@H]5N)c34)cc2F)no1] | N-(4-(3-(((1R,2R)-2- aminocyclohexyl)ami- no)-1H- pyrazolo[3,4-b]pyridin- 4-yl)-2-fluorobenzyl)- 5-(tert-butyl)isoxazole- 3-carboxamide | LCMS (ESI) m/z (M + 1): 506.33 ¹H NMR (400 MHz, DMSO) δ 12.60 (s, 1H), 9.37 (t, J = 5.9 Hz, 1H), 8.45 (d, J = 4.7 Hz, 1H), 7.95 (s, 3H), 7.61 (d, J = 11.0 Hz, 1H), 7.53 (d, J = 3.6 Hz, 2H), 6.98 (d, J = 4.8 Hz, 1H), 6.63 (s, 1H), 4.58 (d, J = 5.9 Hz, 2H), 4.31 (s, 1H), 3.67 (s, 1H), 3.08 (s, 1H), 2.00 (dd, J = 23.4, 12.1 Hz, 2H), 1.65 (d, J = 20.1 Hz, 2H), 1.50-1.20 (m, 13H) |
| 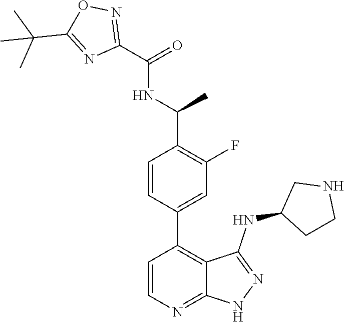 [SMILES: C[C@H](NC(=O)c1noc(C(C)(C)C)n1)c1ccc(-c2ccnc3[nH]nc(N[C@@H]4CCNC4)c23)cc1F] | 5-(tert-butyl)-N-((S)-1- (2-fluoro-4-(3-(((R)- pyrrolidin-3-yl)amino)- 1H- pyrazolo[3,4-b]pyridin- 4-yl)phenyl)ethyl)-1,2,4- oxadiazole-3- carboxamide | LCMS (ESI) m/z (M + 1): 493.34 ¹H NMR (400 MHz, DMSO) δ 12.75 (s, 1H), 9.56 (d, J = 7.9 Hz, 1H), 9.27 (s, 2H), 8.47 (d, J = 4.8 Hz, 1H), 7.68-7.55 (m, 3H), 7.07 (d, J = 4.8 Hz, 1H), 5.49-5.42 (m, 1H), 4.22-4.15 (m, 1H), 3.38-3.30 (m, 1H), 3.25-3.15 (m, 3H), 2.15 (dt, J = 14.5, 7.2 Hz, 1H), 1.85-1.77 (m, 1H), 1.56 (d, J = 7.0 Hz, 3H), 1.44 (s, 9H) |
| 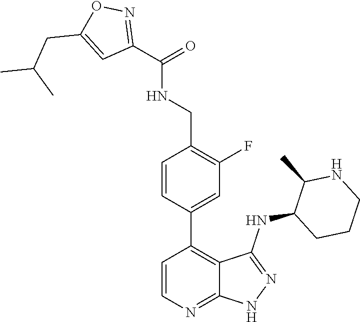 [SMILES: CC(C)Cc1cc(C(=O)NCc2ccc(-c3ccnc4[nH]nc(N[C@@H]5CCCN[C@@H]5C)c34)cc2F)no1] | N-(2-fluoro-4-(3- (((2R,3R)-2- methylpiperidin-3- yl)amino)-1H- pyrazolo[3,4-b]pyridin- 4-yl)benzyl)-5- isobutylisoxazole-3- carboxamide | LCMS (ESI) m/z (M + 1): 506.38 ¹H NMR (400 MHz, DMSO) δ 12.59 (s, 1H), 9.36 (t, J = 6.0 Hz, 1H), 8.99-8.76 (m, 1H), 8.58-8.38 (m, 2H), 7.57-7.47 (m, 3H), 7.01 (d, J = 4.7 Hz, 1H), 6.62 (s, 1H), 4.57 (d, J = 5.9 Hz, 2H), 4.29 (d, J = 6.9 Hz, 1H), 3.99 (s, 1H), 3.67 (s, 1H), 2.94 (s, 2H), 2.72 (d, J = 7.0 Hz, 2H), 2.06-1.96 (m, 1H), 1.72 (s, 2H), 1.57 (s, 2H), 1.14 (d, J = 6.8 Hz, 3H), 0.93 (s, 3H), 0.92 (s, 3H) |
| 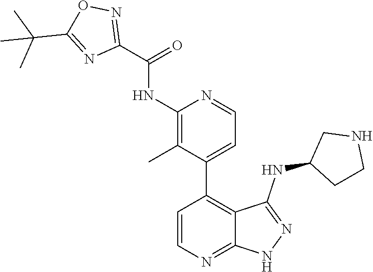 [SMILES: Cc1c(-c2ccnc3[nH]nc(N[C@@H]4CCNC4)c23)ccnc1NC(=O)c1noc(C(C)(C)C)n1] | (R)-5-(tert-butyl)-N-(3- methyl-4-(3-(pyrrolidin- 3-ylamino)-1H- pyrazolo[3,4-b]pyridin- 4-yl)pyridin-2-yl)-1,2,4- oxadiazole-3- carboxamide | LCMS (ESI) m/z (M + 1): 462.33 ¹H NMR (400 MHz, DMSO) δ 12.76 (br, 1H), 11.32 (br, 1H), 9.22 (br, J = 31.5 Hz, 2H), 8.52 (dd, J = 11.2, 4.8 Hz, 2H), 7.40 (dd, J = 20.0, 4.9 Hz, 1H), 6.94 (t, J = 4.8 Hz, 1H), 4.20 (s, 1H), 3.61-2.86 (m, 5H), 2.17 (s, 1H), 2.02 (d, J = 1.5 Hz, 3H), 1.90-1.70 (m, 1H), 1.46 (s, 9H) |
| 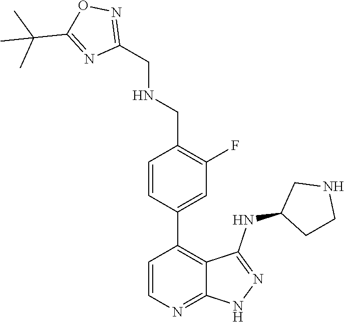 [SMILES: CC(C)(C)c1nc(CNCc2ccc(-c3ccnc4[nH]nc(N[C@@H]5CCNC5)c34)cc2F)no1] | (R)-4-(4-((((5-(tert- butyl)-1,2,4-oxadiazol-3- yl)methyl)amino)meth- yl)-3-fluorophenyl)-N- (pyrrolidin-3-yl)-1H- pyrazolo[3,4-b]pyridin-3- amine | LCMS (ESI) m/z (M + 1): 465.34 ¹H NMR (400 MHz, DMSO) δ 12.72 (s, 1H), 10.33 (s, 3H), 9.46 (s, 1H), 9.25 (s, 1H), 8.49 (d, J = 4.7 Hz, 1H), 7.87 (t, J = 8.0 Hz, 1H), 7.73-7.57 (m, 2H), 7.06 (d, J = 4.8 Hz, 1H), 4.48 (d, J = 17.9 Hz, 4H), 4.24 (s, 2H), 3.36- 3.09 (m, 4H), 2.18-2.07 (m, 1H), 1.95-1.87 (m, J = 4.6 Hz, 1H), 1.43 (s, 9H) |
| 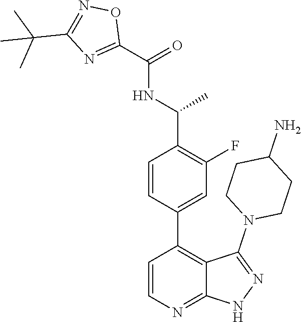 | (R)-N-(1-(4-(3-(4- aminopiperidin-1-yl)- 1H- pyrazolo[3,4-b]pyridin- 4-yl)-2- fluorophenyl)ethyl)-3- (tert-butyl)-1,2,4- oxadiazole-5- carboxamide | LCMS (ESI) m/z (M + 1): 493.59 ¹H NMR (400 MHz, DMSO) δ 13.00 (s, 1H), 9.95 (t, J = 6.0 Hz, 1H), 8.50 (d, J = 4.7 Hz, 1H), 7.94 (d, J = 13.0 Hz, 3H), 7.70-7.45 (m, 3H), 7.13 (d, J = 4.7 Hz, 1H), 4.61 (d, J = 5.9 Hz, 2H), 3.11 (d, J = 12.4 Hz, 2H), 3.06-2.97 (m, 2H), 2.64 (dd, J = 20.5, 8.8 Hz, 2H), 1.69 (d, J = 11.7 Hz, 2H), 1.41 (s, 1H), 1.37 (s, 9H) |
| 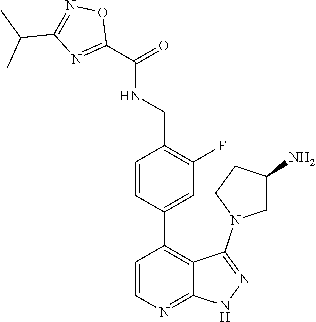 [SMILES: CC(C)c1noc(C(=O)NCc2ccc(-c3ccnc4[nH]nc(N5CC[C@@H](N)C5)c34)cc2F)n1] | (R)-N-(4-(3-(3- aminopyrrolidin-1-yl)- 1H- pyrazolo[3,4-b]pyridin- 4-yl)-2-fluorobenzyl)-3- isopropyl-1,2,4- oxadiazole-5- carboxamide | LCMS (ESI) m/z (M + 1): 465.23 ¹H NMR (400 MHz, DMSO) δ 10.00 (t, J = 5.9 Hz, 1H), 8.51 (d, J = 4.8 Hz, 1H), 8.32 (s, 3H), 7.68-7.43 (m, 3H), 7.14 (d, J = 4.8 Hz, 1H), 4.60 (d, J = 5.8 Hz, 2H), 3.61 (s, 1H), 3.51-3.46 (m, 1H), 3.27 (dd, J = 10.8, 7.1 Hz, 1H), 3.17 (dt, J = 13.8, 6.9 Hz, 1H), 3.01 (dd, J = 10.8, 5.6 Hz, 1H), 2.84 (dd, J = 16.5, 7.4 Hz, 1H), 2.73-2.63 (m, 1H), 1.98 (dd, J = 13.1, 7.6 Hz, 1H), 1.74-1.65 (m, 1H), 1.32 (d, J = 6.9 Hz, 6H) |
| 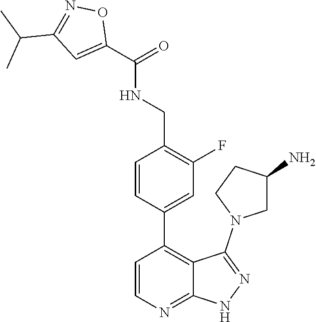 [SMILES: CC(C)c1cc(C(=O)NCc2ccc(-c3ccnc4[nH]nc(N5CC[C@@H](N)C5)c34)cc2F)on1] | (R)-N-(4-(3-(3- aminopyrrolidin-1-yl)- 1H- pyrazolo[3,4-b]pyridin- 4-yl)-2-fluorobenzyl)-3- isopropylisoxazole-5- carboxamide | LCMS (ESI) m/z (M + 1): 464.23 ¹H NMR (400 MHz, DMSO) δ 12.98 (s, 1H), 9.59 (t, J = 5.8 Hz, 1H), 8.50 (d, J = 4.7 Hz, 1H), 8.14 (s, 3H), 7.58-7.42 (m, 3H), 7.18 (s, 1H), 7.12 (d, J = 4.8 Hz, 1H), 4.58 (d, J = 5.8 Hz, 2H), 3.65 (s, 1H), 3.27 (dd, J = 10.8, 7.1 Hz, 1H), 3.07 (dt, J = 13.8, 6.9 Hz, 1H), 2.98 (dd, J = 10.9, 5.4 Hz, 1H), 2.82 (dd, J = 16.7, 7.3 Hz, 1H), 2.71-2.64 (m, 1H), 1.98 (dd, J = 13.0, 7.8 Hz, 1H), 1.66 (dd, J = 12.8, 7.5 Hz, 1H), 1.25 (d, J = 6.9 Hz, 6H) |

### Example 35: (R)—N-(4-(3-((1-acryloylpyrrolidin-3-yl)amino)-1H-pyrazolo[3,4-b]pyridin-4-yl)-2-fluorobenzyl)-5-phenylisoxazole-3-carboxamide

Synthesis of (R)—N-(2-fluoro-4-(1-(4-methoxybenzyl)-3-(pyrrolidin-3-ylamino)-1H-pyrazolo[3,4-b]pyridin-4-yl)benzyl)-5-phenylisoxazole-3-carboxamide (260 mg, 66%) followed a similar procedure outlined in Method 3.4. LCMS (ESI) m/z (M+1): 718.26.

The following compounds were prepared by analogous methods:

| Chemical structure | Chemical names | LC-MS/HNMR |
|---|---|---|
| 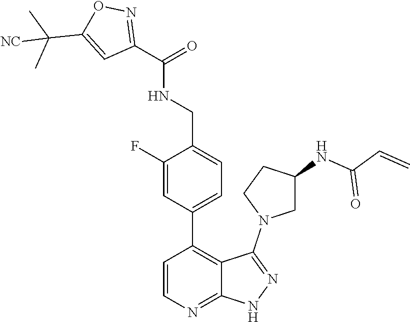 [SMILES: C=CC(=O)N[C@@H]1CCN(c2n[nH]c3nccc(-c4ccc(CNC(=O)c5cc(C(C)(C)C#N)on5)c(F)c4)c23)C1] | (R)-N-(4-(3-(3- acrylamidopyrrolidin- 1-yl)-1H-pyrazolo- [3,4-b]pyridin-4-yl)- 2-fluorobenzyl)-5-(2- cyanopropan-2-yl)- isoxazole-3- carboxamide | LCMS (ESI) m/z (M + 1): 543.22 ¹H NMR (400 MHz, DMSO) δ 12.85 (s, 1H), 9.46 (t, J = 5.9 Hz, 1H), 8.46 (d, J = 4.7 Hz, 1H), 8.16 (d, J = 7.0 Hz, 1H), 7.55-7.44 (m, 3H), 7.08 (d, J = 4.7 Hz, 1H), 7.00 (s, 1H), 6.12 (ddd, J = 19.4, 17.1, 6.2 Hz, 2H), 5.56 (dd, J = 10.0, 2.3 Hz, 1H), 4.57 (d, J = 4.6 Hz, 2H), 4.17 (dd, J = 13.0, 6.1 Hz, 1H), 3.12 (dd, J = 10.1, 6.9 Hz, 1H), 2.85 (dd, J = 16.6, 7.4 Hz, 1H), 2.77-2.68 (m, 2H), 2.02-1.89 (m, 1H), 1.78 (s, 6H), 1.52 (dd, J = 12.8, 7.4 Hz, 1H) |
| 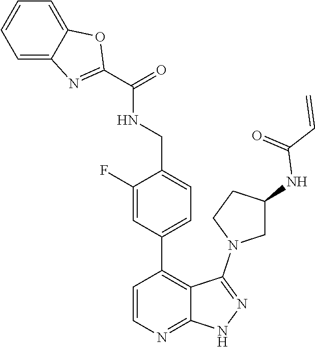 [SMILES: C=CC(=O)N[C@@H]1CCN(c2n[nH]c3nccc(-c4ccc(CNC(=O)c5nc6ccccc6o5)c(F)c4)c23)C1] | (R)-N-(4-(3-(3- acrylamidopyrrolidin- 1-yl)-1H-pyrazolo- [3,4-b]pyridin-4-yl)- 2-fluorobenzyl)- benzo[d]-oxazole-2- carboxamide | LCMS (ESI) m/z (M + 1): 526.26 ¹H NMR (400 MHz, DMSO) δ 12.85 (s, 1H), 9.87 (t, J = 6.1 Hz, 1H), 8.46 (d, J = 4.7 Hz, 1H), 8.15 (d, J = 7.0 Hz, 1H), 7.89 (dd, J = 14.3, 7.8 Hz, 2H), 7.70-7.33 (m, 5H), 7.08 (d, J = 4.7 Hz, 1H), 6.11 (ddd, J = 19.4, 17.1, 6.2 Hz, 2H), 5.52 (dd, J = 10.1, 2.3 Hz, 1H), 4.64 (d, J = 6.0 Hz, 2H), 4.18 (dd, J = 13.0, 6.4 Hz, 1H), 3.13 (dd, J = 10.1, 6.9 Hz, 1H), 2.92-2.82 (m, 1H), 2.75 (dt, J = 10.2, 5.0 Hz, 2H), 1.96 (td, J = 14.7, 7.5 Hz, 1H), 1.54 (td, J = 13.0, 5.6 Hz, 1H) |
| 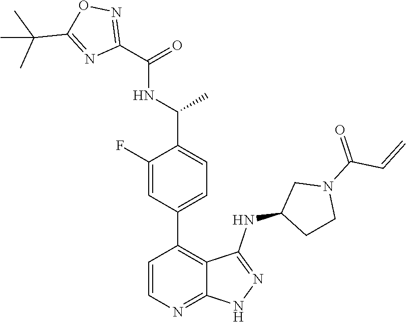 [SMILES: C=CC(=O)N1CC[C@@H](Nc2n[nH]c3nccc(-c4ccc([C@@H](C)NC(=O)c5noc(C(C)(C)C)n5)c(F)c4)c23)C1] | N-((R)-1-(4-(3-(((R)- 1-acryloylpyrrolidin- 3-yl)amino)-1H- pyrazolo[3,4-b]- pyridin-4-yl)-2- fluorophenyl)ethyl)- 5-(tert-butyl)-1,2,4- oxadiazole-3- carboxamide | LCMS (ESI) m/z (M + 1): 547.37 ¹H NMR (400 MHz, DMSO) δ 12.59 (s, 1H), 9.53 (dd, J = 7.7, 5.7 Hz, 1H), 8.45-8.43 (m, 1H), 7.71-7.59 (m, 1H), 7.54-7.41 (m, 2H), 7.01 (dd, J = 4.7, 2.2 Hz, 1H), 6.53 (ddd, J = 40.1, 16.7, 10.3 Hz, 1H), 6.10 (ddd, J = 16.8, 10.5, 2.4 Hz, 1H), 5.62 (ddd, J = 23.3, 10.3, 2.4 Hz, 1H), 5.53-5.40 (m, 1H), 4.49 (dt, J = 11.4, 5.8 Hz, 1H), 4.13 (dd, J = 26.6, 4.8 Hz, 1H), 3.82 (dd, J = 10.4, 5.7 Hz, 0.5H), 3.58 (dd, J = 15.3, 6.6 Hz, 1.5H), 3.44 (dd, J = 12.9, 5.9 Hz, 1H), 2.24-2.03 (m, 1H), 1.83 (ddd, J = 19.2, 12.1, 6.2 Hz, 1H), 1.54 (d, J = 6.9 Hz, 3H), 1.43 (s, 9H) |
| 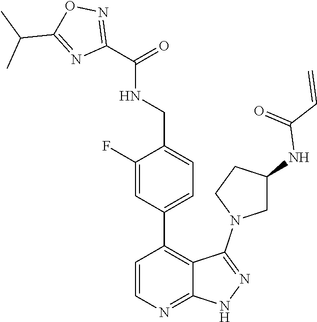 [SMILES: C=CC(=O)N[C@@H]1CCN(c2n[nH]c3nccc(-c4ccc(CNC(=O)c5noc(C(C)C)n5)c(F)c4)c23)C1] | (R)-N-(4-(3-(3- acrylamidopyrrolidin- 1-yl)-1H-pyrazolo- [3,4-b]pyridin-4-yl)- 2-fluorobenzyl)-5- isopropyl-1,2,4- oxadiazole-3- carboxamide | LCMS (ESI) m/z (M + 1): 519.24 ¹H NMR (400 MHz, DMSO) δ 12.84 (s, 1H), 9.52 (t, J = 5.9 Hz, 1H), 8.46 (d, J = 4.7 Hz, 1H), 8.14 (d, J = 7.1 Hz, 1H), 7.50 (dd, J = 14.5, 7.5 Hz, 2H), 7.08 (d, J = 4.7 Hz, 1H), 6.12 (ddd, J = 19.3, 17.1, 6.2 Hz, 2H), 5.56 (dd, J = 10.0, 2.1 Hz, 1H), 4.58 (s, 2H), 4.16 (d, J = 6.9 Hz, 1H), 3.44-3.32 (m, 1H), 3.13 (dd, J = 10.0, 6.9 Hz, 1H), 2.85 (d, J = 8.8 Hz, 1H), 2.79-2.65 (m, 2H), 2.06-1.87 (m, 1H), 1.52 (d, J = 5.8 Hz, 1H), 1.36 (d, J = 7.0 Hz, 6H) |
| 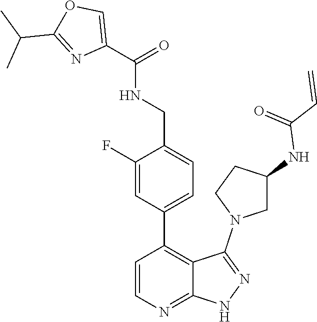 [SMILES: C=CC(=O)N[C@@H]1CCN(c2n[nH]c3nccc(-c4ccc(CNC(=O)c5coc(C(C)C)n5)c(F)c4)c23)C1] | (R)-N-(4-(3-(3- acrylamidopyrrolidin- 1-yl)-1H-pyrazolo- [3,4-b]pyridin-4-yl)- 2-fluorobenzyl)-2- isopropyloxazole-4- carboxamide | LCMS (ESI) m/z (M + 1): 518.28 ¹H NMR (400 MHz, DMSO) δ 12.86 (s, 1H), 8.74 (t, J = 6.1 Hz, 1H), 8.53 (s, 1H), 8.46 (d, J = 4.7 Hz, 1H), 8.17 (d, J = 7.0 Hz, 1H), 7.55-7.41 (m, 3H), 7.07 (d, J = 4.7 Hz, 1H), 6.13 (ddd, J = 19.4, 17.1, 6.2 Hz, 2H), 5.56 (dd, J = 10.0, 2.3 Hz, 1H), 4.54 (d, J = 5.0 Hz, 2H), 4.23- 4.11 (m, 1H), 3.20-3.07 (m, 2H), 2.88-2.66 (m, 3H), 1.99-1.87 (m, 1H), 1.60-1.43 (m, 1H), 1.30 (d, J = 7.0 Hz, 6H) |
| 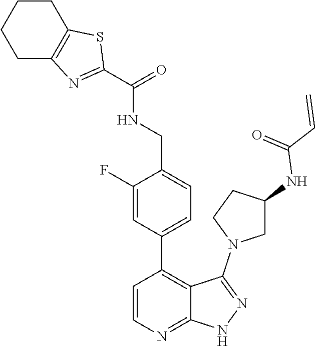 [SMILES: C=CC(=O)N[C@@H]1CCN(c2n[nH]c3nccc(-c4ccc(CNC(=O)c5nc6c(s5)CCCC6)c(F)c4)c23)C1] | (R)-N-(4-(3-(3- acrylamidopyrrolidin- 1-yl)-1H-pyrazolo- [3,4-b]pyridin-4-yl)- 2-fluorobenzyl)- 4,5,6,7-tetrahydro- benzo[d]thiazole- 2-carboxamide | LCMS (ESI) m/z (M + 1): 546.23 ¹H NMR (400 MHz, DMSO) δ 12.83 (s, 1H), 9.29 (t, J = 6.1 Hz, 1H), 8.46 (d, J = 4.7 Hz, 1H), 8.13 (d, J = 6.9 Hz, 1H), 7.56-7.35 (m, 3H), 7.07 (d, J = 4.7 Hz, 1H), 6.13 (ddd, J = 19.3, 17.1, 6.2 Hz, 2H), 5.56 (dd, J = 10.0, 2.3 Hz, 1H), 4.56 (d, J = 6.1 Hz, 2H), 4.27-4.08 (m, 1H), 3.14 (dd, J = 10.0, 7.0 Hz, 1H), 2.92-2.61 (m, 6H), 2.61-2.48 (m, 2H), 1.92 (dd, J = 12.7, 7.1 Hz, 1H), 1.82 (d, J = 5.1 Hz, 3H), 1.59- 1.44 (m, 1H) |
| 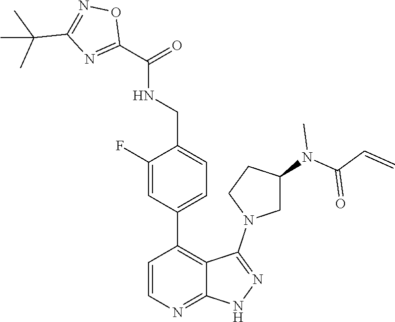 [SMILES: C=CC(=O)N(C)[C@@H]1CCN(c2n[nH]c3nccc(-c4ccc(CNC(=O)c5nc(C(C)(C)C)no5)c(F)c4)c23)C1] | (R)-3-(tert-butyl)-N- (2-fluoro-4-(3-(3-(N- methylacrylamido)- pyrrolidin-1-yl)-1H- pyrazolo[3,4-b]- pyridin-4-yl)benzyl)- 1,2,4-oxadiazole-5- carboxamide | LCMS (ESI) m/z (M + 1): 547.29 ¹H NMR (400 MHz, DMSO) δ 12.92 (s, 1H), 9.93 (s, 1H), 8.48 (d, J = 4.7 Hz, 1H), 7.87-7.39 (m, 3H), 7.10 (d, J = 4.7 Hz, 1H), 6.80-6.49 (m, 1H), 6.06 (dd, J = 29.3, 17.6 Hz, 1H), 5.73-5.51 (m, 1H), 5.11 (s, 1H), 4.59 (d, J = 5.4 Hz, 2H), 3.25-3.01 (m, 1H), 2.92 (s, 3H), 2.79-2.62 (m, 3H), 1.97-1.77 (m, 1H), 1.72-1.43 (m, 1H), 1.36 (s, J = 5.4 Hz, 9H) |
| 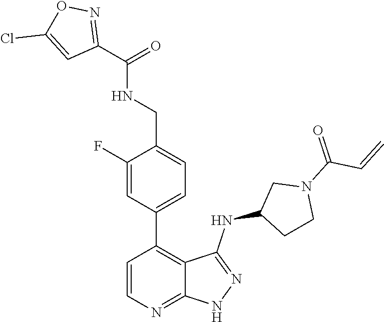 | (R)-N-(4-(3-((1- acryloylpyrrolidin- 3-yl)amino)-1H- pyrazolo[3,4-b]- pyridin-4-yl)-2- fluorobenzyl)-5- chloroisoxazole-3- carboxamide | LCMS (ESI) m/z (M + 1): 510.29 ¹H NMR (400 MHz, DMSO) δ 12.58 (s, 1H), 9.51 (d, J = 3.1 Hz, 1H), 8.43 (t, J = 5.2 Hz, 1H), 7.52-7.47 (m, 1H), 7.43 (d, J = 7.9 Hz, 1H), 7.12 (d, J = 4.2 Hz, 1H), 7.00 (dd, J = 4.7, 1.0 Hz, 1H), 6.52 (ddd, J = 29.8, 16.8, 10.3 Hz, 1H), 6.11 (ddd, J = 16.8, 7.1, 2.4 Hz, 1H), 5.62 (ddd, J = 12.6, 10.4, 2.4 Hz, 1H), 4.56 (d, J = 5.8 Hz, 1H), 4.54-4.47 (m, 1H), 4.24-4.07 (m, 1H), 3.83 (dd, J = 10.5, 6.1 Hz, 0.5H), 3.67-3.54 (m, 1H), 3.51-3.36 (m, 2.5H), 2.24- 2.03 (m, 1H), 1.94 - 1.71 (m, 1H). |
| 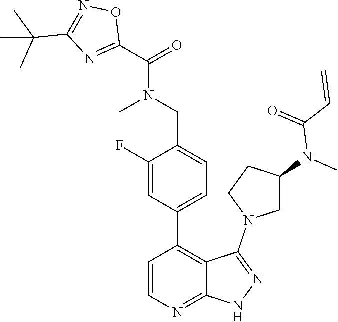 [SMILES: C=CC(=O)N(C)[C@@H]1CCN(c2n[nH]c3nccc(-c4ccc(CN(C)C(=O)c5nc(C(C)(C)C)no5)c(F)c4)c23)C1] | (R)-3-(tert-butyl)-N- (2-fluoro)-4-(3-(3-(N- methylacrylamido)- pyrrolidin-1-yl)-1H- pyrazolo[3,4-b]- pyridin-4-yl)benzyl)- N-methyl-1,2,4- oxadiazole-5- carboxamide | LCMS (ESI) m/z (M + 1): 561.26 ¹H NMR (400 MHz, DMSO) δ 12.94 (s, 1H), 8.49 (dd, J = 4.7, 2.5 Hz, 1H), 7.70-7.42 (m, 3H), 7.11 (dd, J = 8.1, 4.7 Hz, 1H), 6.69 (d, J = 14.8 Hz, 1H), 6.03 (d, J = 17.6 Hz, 1H), 5.65 (d, J = 10.5 Hz, 1H), 5.10-4.68 (m, 3H), 3.11 (d, J = 49.2 Hz, 4H), 2.86 (d, J = 64.2 Hz, 4H), 2.67 (s, 2H), 1.88 (s, 1H), 1.53 (s, 1H), 1.37 (t, J = 31.5 Hz, 9H). |
| 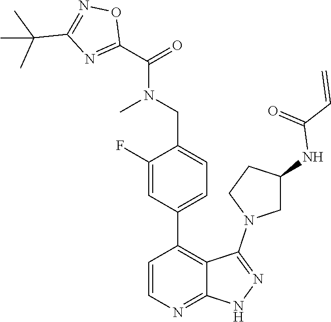 [SMILES: C=CC(=O)N[C@@H]1CCN(c2n[nH]c3nccc(-c4ccc(CN(C)C(=O)c5nc(C(C)(C)C)no5)c(F)c4)c23)C1] | (R)-N-(4-(3-(3- acrylamidopyrrolidin- 1-yl)-1H-pyrazolo- [3,4-b]pyridin-4-yl)- 2-fluorobenzyl)-3- (tert-butyl)-N-methyl- 1,2,4-oxadiazole-5- carboxamide | LCMS (ESI) m/z (M + 1): 547.26 ¹H NMR (400 MHz, DMSO) δ 12.87 (s, 1H), 8.47 (dd, J = 4.7, 3.4 Hz, 1H), 8.14 (d, J = 6.9 Hz, 1H), 7.66- 7.44 (m, 3H), 7.09 (dd, J = 11.5, 4.7 Hz, 1H), 6.17 (dd, J = 17.1, 10.1 Hz, 1H), 6.03 (ddd, J = 17.1, 8.1, 2.3 Hz, 1H), 5.58-5.50 (m, 1H), 4.94 (q, J = 16.2 Hz, 1H), 4.85 (s, 1H), 4.15 (s, 1H), 3.18-2.97 (m, 4H), 2.89-2.81 (m, 1H), 2.79-2.67 (m, 2H), 1.94 (td, J = 13.1, 6.9 Hz, 1H), 1.53 (dt, J = 12.8, 6.4 Hz, 1H), 1.33 (d, J = 16.3 Hz, 9H). |
| 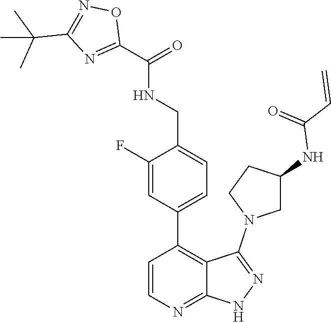 [SMILES: C=CC(=O)N[C@@H]1CCN(c2n[nH]c3nccc(-c4ccc(CNC(=O)c5nc(C(C)(C)C)no5)c(F)c4)c23)C1] | (R)-N-(4-(3-(3- acrylamidopyrrolidin- 1-yl)-1H-pyrazolo- [3,4-b]pyridin-4-yl)- 2-fluorobenzyl)-3- (tert-butyl)-1,2,4- oxadiazole-5- carboxamide | LCMS (ESI) m/z (M + 1): 533.38 ¹H NMR (400 MHz, DMSO) δ 12.85 (s, 1H), 9.89 (t, J = 5.9 Hz, 1H), 8.47 (d, J = 4.7 Hz, 1H), 8.15 (d, J = 7.0 Hz, 1H), 7.60-7.44 (m, 3H), 7.08 (d, J = 4.7 Hz, 1H), 6.19 (dd, J = 17.1, 10.1 Hz, 1H), 6.04 (dd, J = 17.1, 2.3 Hz, 1H), 5.55 (dd, J = 10.0, 2.3 Hz, 1H), 4.66- 4.53 (m, 2H), 4.17 (dt, J = 13.0, 6.7 Hz, 1H), 3.12 (dd, J = 10.1, 6.9 Hz, 1H), 2.86 (dd, J = 16.6, 7.6 Hz, 1H), 2.78-2.68 (m, 2H), 1.94 (dt, J = 14.8, 7.6 Hz, 1H), 1.52 (dt, J = 9.3, 6.6 Hz, 1H), 1.36 (s, 9H). |
| 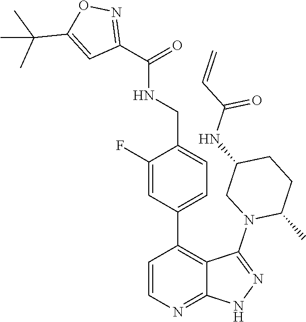 [SMILES: C=CC(=O)N[C@@H]1CC[C@H](C)N(c2n[nH]c3nccc(-c4ccc(CNC(=O)c5cc(C(C)(C)C)on5)c(F)c4)c23)C1] | N-(4-(3-((2S,5R)-5- acrylamido-2- methylpiperidin-1- yl)-1H-pyrazolo- [3,4-b]pyridin-4-yl)- 2-fluorobenzyl)-5- (tert-butyl)isoxazole- 3-carboxamide | LCMS (ESI) m/z (M + 1): 560.32 ¹H NMR (400 MHz, DMSO) δ 12.93 (s, 1H), 9.33 (t, J = 6.0 Hz, 1H), 8.47 (d, J = 4.7 Hz, 1H), 8.02 (d, J = 8.0 Hz, 1H), 7.59-7.46 (m, 3H), 7.10 (d, J = 4.7 Hz, 1H), 6.57 (s, 1H), 6.20 (dd, J = 17.1, 10.0 Hz, 1H), 6.08 (dd, J = 17.1, 2.4 Hz, 1H), 5.59 (dd, J = 9.9, 2.4 Hz, 1H), 4.62-4.51 (m, 2H), 3.79 (s, 1H), 3.25-3.19 (m, 1H), 2.93-2.83 (m, 2H), 1.56-1.48 (m, 1H), 1.47-1.10 (m, 11H), 1.05 (d, J = 12.2 Hz, 1H), 0.64 (d, J = 6.7 Hz, 3H) |
| 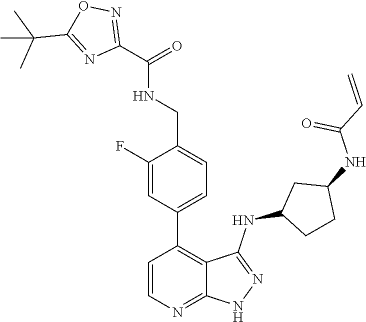 | N-(4-(3-(((1R,3S)-3- acrylamidocyclo- pentyl)amino)-1H- pyrazolo[3,4-b]- pyridin-4-yl)-2- fluorobenzyl)-5- (tert-butyl)-1,2,4- oxadiazole-3- carboxamide | LCMS (ESI) m/z (M + 1): 547.34 ¹H NMR (400 MHz, DMSO) δ 12.46 (s, 1H), 9.55 (t, J = 6.0 Hz, 1H), 8.42 (d, J = 4.7 Hz, 1H), 8.05 (d, J = 7.1 Hz, 1H), 7.59-7.42 (m, 3H), 6.96 (d, J = 4.7 Hz, 1H), 6.17 (dd, J = 17.1, 10.0 Hz, 1H), 6.03 (dd, J = 17.1, 2.4 Hz, 1H), 5.53 (dd, J = 10.0, 2.4 Hz, 1H), 4.59 (d, J = 6.0 Hz, 2H), 4.23 (d, J = 6.3 Hz, 1H), 4.03 (dd, J = 14.5, 7.1 Hz, 1H), 3.87 (dd, J = 12.7, 6.1 Hz, 1H), 2.37-2.31 (m, 1H), 1.95- 1.81 (m, 2H), 1.64-1.16 (m, 12H) |
| 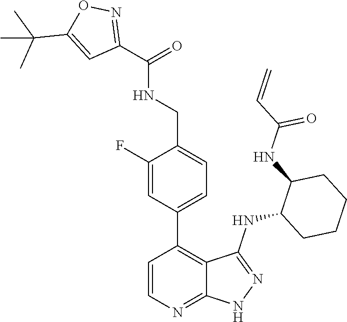 [SMILES: C=CC(=O)N[C@H]1CCCC[C@@H]1Nc1n[nH]c2nccc(-c3ccc(CNC(=O)c4cc(C(C)(C)C)on4)c(F)c3)c12] | N-(4-(3-(((1S,2S)-2- acrylamidocyclo- hexyl)amino)-1H- pyrazolo[3,4-b]- pyridin-4-yl)-2- fluorobenzyl)-5- (tert-butyl)isoxazole- 3-carboxamide | LCMS (ESI) m/z (M + 1): 560.35 ¹H NMR (400 MHz, DMSO) δ 12.35 (s, 1H), 9.21 (t, J = 5.8 Hz, 1H), 8.39 (d, J = 4.7 Hz, 1H), 7.98 (d, J = 8.4 Hz, 1H), 7.49 (t, J = 7.8 Hz, 1H), 7.33 (dd, J = 10.7, 1.5 Hz, 1H), 7.26 (dd, J = 7.8, 1.6 Hz, 1H), 6.89 (d, J = 4.7 Hz, 1H), 6.60 (s, 1H), 6.05 (dd, J = 17.1, 10.0 Hz, 1H), 5.93 (dd, J = 17.1, 2.4 Hz, 1H), 5.45 (dd, J = 10.0, 2.4 Hz, 1H), 4.61-4.50 (m, 2H), 4.06 (d, J = 5.8 Hz, 1H), 3.58 (s, 1H), 3.43- 3.37 (m, 1H), 2.37 (d, J = 11.3 Hz, 1H), 1.78 (s, 1H), 1.62 (d, J= 16.2 Hz, 2H), 1.43-0.93 (m, 13H) |
| 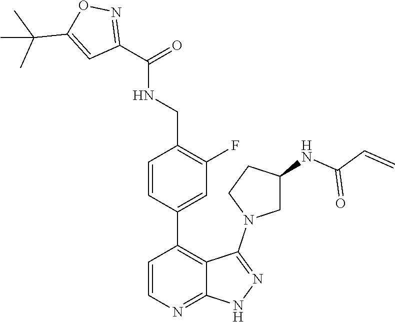 [SMILES: C=CC(=O)N[C@@H]1CCN(c2n[nH]c3nccc(-c4ccc(CNC(=O)c5cc(C(C)(C)C)on5)c(F)c4)c23)C1] | (R)-N-(4-(3-(3- acrylamidopyrrolidin- 1-yl)-1H-pyrazolo- [3,4-b]pyridin-4-yl)- 2-fluorobenzyl)-5- (tert-butyl)isoxazole- 3-carboxamide | LCMS (ESI) m/z (M + 1): 532.49 ¹H NMR (400 MHz, DMSO) δ 12.84 (s, 1H), 9.30 (t, J = 5.9 Hz, 1H), 8.46 (d, J = 4.7 Hz, 1H), 8.16 (d, J = 6.9 Hz, 1H), 7.56-7.42 (m, 3H), 7.07 (d, J = 4.7 Hz, 1H), 6.58 (s, 1H), 6.19 (dd, J = 17.1, 10.1 Hz, 1H), 6.05 (dd, J = 17.1, 2.3 Hz, 1H), 5.56 (dd, J = 10.1, 2.3 Hz, 1H), 4.63-4.49 (m, 2H), 4.17 (dd, J = 12.8, 6.4 Hz, 1H), 3.13 (dd, J = 10.1, 6.9 Hz, 1H), 2.87-2.80 (m, 1H), 2.78-2.67 (m, 2H), 1.93 (td, J = 14.8, 7.5 Hz, 1H), 1.52 (dt, J = 13.8, 6.2 Hz, 1H), 1.33 (s, 9H). |
| 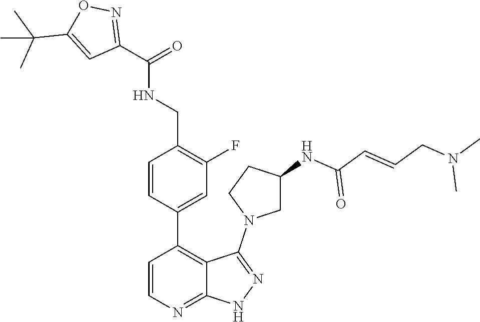 [SMILES: CN(C)C/C=C/C(=O)N[C@@H]1CCN(c2n[nH]c3nccc(-c4ccc(CNC(=O)c5cc(C(C)(C)C)on5)c(F)c4)c23)C1] | (R,E)-5-(tert-butyl)- N-(4-(3-(3-(4- (dimethylamino)but- 2-enamido)pyrrolidin- 1-yl)-1H-pyrazolo- [3,4-b]pyridin-4-yl)- 2-fluorobenzyl)- isoxazole-3- carboxamide | LCMS (ESI) m/z (M + 1): 589.45 ¹H NMR (400 MHz, DMSO) δ 12.84 (s, 1H), 9.31 (t, J = 5.9 Hz, 1H), 8.46 (d, J = 4.7 Hz, 1H), 8.06 (d, J = 7.0 Hz, 1H), 7.56-7.43 (m, 3H), 7.07 (d, J = 4.7 Hz, 1H), 6.62-6.48 (m, 2H), 6.01 (d, J = 15.5 Hz, 1H), 4.56 (d, J = 5.8 Hz, 2H), 4.16 (dd, J = 13.0, 6.7 Hz, 1H), 3.13-3.07 (m, 1H), 2.98 (d, J = 5.5 Hz, 2H), 2.88-2.82 (m, 1H), 2.75-2.68 (m, 2H), 2.14 (s, 6H), 1.92 (dt, J = 14.8, 7.6 Hz, 1H), 1.55-1.48 (m, 1H), 1.33 (s, 9H). |
| 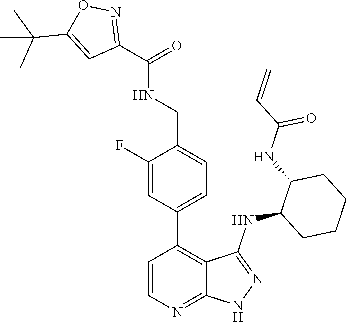 [SMILES: C=CC(=O)N[C@@H]1CCCC[C@H]1Nc1n[nH]c2nccc(-c3ccc(CNC(=O)c4cc(C(C)(C)C)on4)c(F)c3)c12] | N-(4-(3-(((1R,2R)-2- acrylamidocyclo- hexyl)amino)-1H- pyrazolo[3,4-b]- pyridin-4-yl)-2- fluorobenzyl)-5- (tert-butyl)isoxazole- 3-carboxamide | LCMS (ESI) m/z (M + 1): 560.38 ¹H NMR (400 MHz, DMSO) δ 12.42 (s, 1H), 9.22 (t, J = 5.7 Hz, 1H), 8.41 (d, J = 4.8 Hz, 1H), 8.01 (d, J = 8.3 Hz, 1H), 7.50 (t, J = 7.8 Hz, 1H), 7.34 (dd, J = 10.6, 1.3 Hz, 1H), 7.27 (dd, J = 7.8, 1.5 Hz, 1H), 6.91 (d, J = 4.8 Hz, 1H), 6.61 (s, 1H), 6.06 (dd, J = 17.1, 10.0 Hz, 1H), 5.94 (dd, J = 17.1, 2.4 Hz, 1H), 5.46 (dd, J = 10.0, 2.4 Hz, 1H), 4.64-4.51 (m, 2H), 3.63- 3.55 (m, 1H), 3.41 (td, J = 10.2, 3.7 Hz, 1H), 2.36 (d, J = 12.0 Hz, 1H), 1.79 (s, 1H), 1.62 (d, J = 14.5 Hz, 2H), 1.55-0.65 (m, 14H) |
| 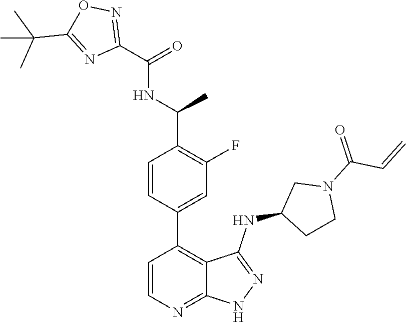 [SMILES: C=CC(=O)N1CC[C@@H](Nc2n[nH]c3nccc(-c4ccc([C@H](C)NC(=O)c5noc(C(C)(C)C)n5)c(F)c4)c23)C1] | N-((S)-1-(4-(3-(((R)- 1-acryloylpyrrolidin- 3-yl)amino)-1H- pyrazolo[3,4-b]- pyridin-4-yl)-2- fluorophenyl)ethyl)- 5-(tert-butyl)-1,2,4- oxadiazole-3- carboxamide | LCMS (ESI) m/z (M + 1): 547.63 ¹H NMR (400 MHz, DMSO) δ 12.58 (s, 1H), 9.52 (dd, J = 7.8, 2.3 Hz, 1H), 8.44 (d, J = 4.6 Hz, 1H), 7.62 (dd, J = 15.3, 7.6 Hz, 1H), 7.47 (dd, J = 14.4, 9.8 Hz, 2H), 7.01 (dd, J = 4.6, 1.9 Hz, 1H), 6.51 (ddd, J = 23.7, 16.8, 10.3 Hz, 1H), 6.11 (ddd, J = 16.8, 8.0, 2.3 Hz, 1H), 5.62 (ddd, J = 16.0, 10.3, 2.3 Hz, 1H), 5.51-5.38 (m, 1H), 4.47 (d, J = 5.5 Hz, 1H), 4.22-4.07 (m, 1H), 3.83- 3.54 (m, 2H), 3.47-3.38 (m, 2H), 2.22-2.01 (m, 1H), 1.91-1.72 (m, 1H), 1.55 (d, J = 6.9 Hz, 3H), 1.43 (s, 9H) |
| 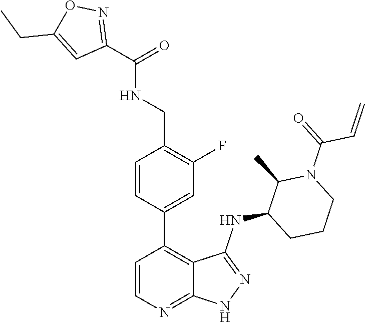 [SMILES: C=CC(=O)N1CCC[C@@H](Nc2n[nH]c3nccc(-c4ccc(CNC(=O)c5cc(CC)on5)c(F)c4)c23)[C@H]1C] | N-(4-(3-(((2R,3R)- 1-acryloyl-2- methylpiperidin-3- yl)amino)-1H- pyrazolo[3,4-b]- pyridin-4-yl)-2- fluorobenzyl)-5- ethylisoxazole-3- carboxamide | LCMS (ESI) m/z (M + 1): 532.61 ¹H NMR (400 MHz, DMSO) δ 12.51 (s, 1H), 9.35 (t, J = 5.9 Hz, 1H), 8.43 (d, J = 4.7 Hz, 1H), 7.67-7.40 (m, 3H), 6.98 (d, J = 4.7 Hz, 1H), 6.77-6.65 (m, 1H), 6.60 (s, 1H), 6.06 (d, J = 16.0 Hz, 1H), 5.64 (d, J = 11.0 Hz, 1H), 5.03-4.78 (m, 1H), 4.56 (d, J = 5.9 Hz, 2H), 4.23- 3.54 (m, 4H), 2.82 (q, J = 7.6 Hz, 2H), 1.72 (s, 1H), 1.61 (s, 1H), 1.35 (s, 2H), 1.24 (t, J = 7.6 Hz, 3H), 0.92 (s, 3H) |
| 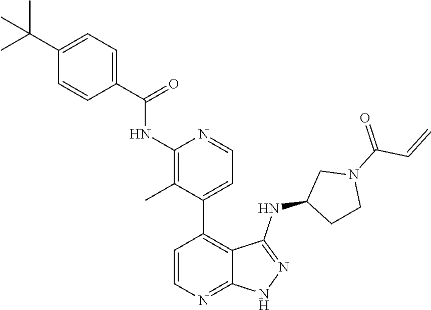 [SMILES: C=CC(=O)N1CC[C@@H](Nc2n[nH]c3nccc(-c4ccnc(NC(=O)c5ccc(C(C)(C)C)cc5)c4C)c23)C1] | (R)-N-(4-(3-((1- acryloylpyrrolidin- 3-yl)amino)-1H- pyrazolo[3,4-b]- pyridin-4-yl)-3- methylpyridin-2- yl)-4-(tert-butyl)- benzamide | LCMS (ESI) m/z (M + 1): 524.63 ¹H NMR (400 MHz, DMSO) δ 12.61 (s, 1H), 10.91-10.79 (m, 1H), 8.56- 8.37 (m, 3H), 8.02-7.93 (m, 2H), 7.55 (d, J = 8.4 Hz, 2H), 7.34-7.21 (m, 1H), 6.95-6.87 (m, 1H), 6.62- 6.41 (m, 1H), 6.14-6.01 (m, 1H), 5.74-5.38 (m, 1H), 4.31-3.41 (m, 6H), 2.27-2.01 (m, 2H), 1.97-1.88 (m, 3H), 1.32 (s, 9H) |
| 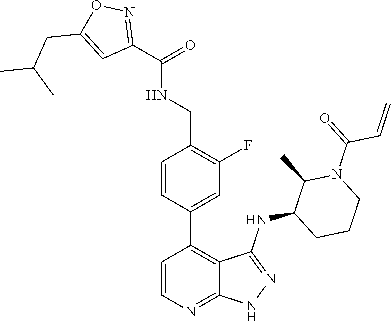 [SMILES: C=CC(=O)N1CCC[C@@H](Nc2n[nH]c3nccc(-c4ccc(CNC(=O)c5cc(CC(C)C)on5)c(F)c4)c23)[C@H]1C] | N-(4-(3-(((2R,3R)- 1-acryloyl-2- methylpiperidin- 3-yl)amino)-1H- pyrazolo[3,4-b]- pyridin-4-yl)-2- fluorobenzyl)-5- isobutylisoxazole- 3-carboxamide | LCMS (ESI) m/z (M + 1): 560.38 ¹H NMR (400 MHz, DMSO) δ 12.51 (s, 1H), 9.36 (t, J = 6.1 Hz, 1H), 8.43 (d, J = 4.7 Hz, 1H), 7.56 (t, J = 7.8 Hz, 1H), 7.52-7.41 (m, 2H), 6.98 (d, J = 4.7 Hz, 1H), 6.79-6.64 (m, 1H), 6.60 (s, 1H), 6.06 (d, J = 17.3 Hz, 1H), 5.64 (d, J = 10.4 Hz, 1H), 5.06-3.77 (m, 2H), 4.56 (d, J = 6.0 Hz, 2H), 3.55 (s, 1H), 2.95 (s, 1H), 2.71 (d, J = 7.0 Hz, 3H), 1.99 (dt, J = 13.5, 6.8 Hz, 1H), 1.73 (s, 1H), 1.62 (d, J = 7.3 Hz, 1H), 1.34 (s, 2H), 0.91 (d, J = 6.7 Hz, 8H) |
| 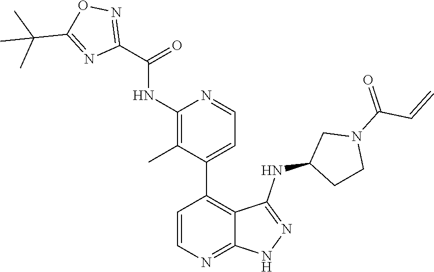 [SMILES: C=CC(=O)N1CC[C@@H](Nc2n[nH]c3nccc(-c4ccnc(NC(=O)c5noc(C(C)(C)C)n5)c4C)c23)C1] | (R)-N-(4-(3-((1- acryloylpyrrolidin- 3-yl)amino)-1H- pyrazolo[3,4-b]- pyridin-4-yl)-3- methylpyridin-2- yl)-5-(tert-butyl)- 1,2,4-oxadiazole- 3-carboxamide | LCMS (ESI) m/z (M + 1): 516.35 ¹H NMR (400 MHz, DMSO) δ 12.63 (d, J = 4.2 Hz, 1H), 11.19 (s, 1H), 8.51 (d, J = 4.6 Hz, 1H), 8.45 (d, J = 4.9 Hz, 1H), 7.39-7.23 (m, 1H), 6.93-6.85 (m, 1H), 6.58-6.41 (m, 1H), 6.12-6.02 (m, 1H), 5.71- 5.41 (m, 1H), 4.20-4.06 (m, 1H), 3.95-3.50 (m, 3H), 3.44-3.37 (m, 1H), 3.27-3.17 (m, 1H), 2.24-2.04 (m, 1H), 1.96 (dd, J = 20.5, 15.0 Hz, 3H), 1.86-1.69 (m, 1H), 1.46 (s, 9H) |
| 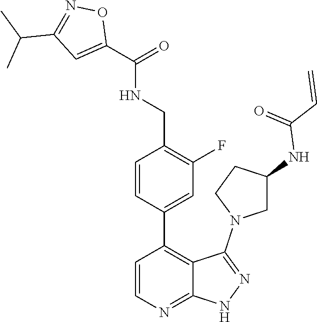 [SMILES: C=CC(=O)N[C@@H]1CCN(c2n[nH]c3nccc(-c4ccc(CNC(=O)c5cc(C(C)C)no5)c(F)c4)c23)C1] | (R)-N-(4-(3-(3- acrylamidopyrrolidin- 1-yl)-1H-pyrazolo- [3,4-b]pyridin-4-yl)- 2-fluorobenzyl)-3- isopropylisoxazole- 5-carboxamide | LCMS (ESI) m/z (M + 1): 518.29 ¹H NMR (400 MHz, DMSO) δ 12.84 (s, 1H), 9.47 (t, J = 5.8 Hz, 1H), 8.46 (d, J = 4.7 Hz, 1H), 8.16 (d, J = 7.0 Hz, 1H), 7.60-7.40 (m, 3H), 7.09 (s, 1H), 7.07 (d, J = 4.7 Hz, 1H), 6.12 (dd, J = 17.1, 6.2 Hz, 2H), 5.56 (dd, J = 10.0, 2.4 Hz, 1H), 4.60-4.50 (m, 2H), 4.17 (dd, J = 12.8, 6.3 Hz, 1H), 3.18-3.01 (m, 2H), 2.88-2.81 (m, 1H), 2.79-2.66 (m, 2H), 2.00-1.88 (m, 1H), 1.52 (dt, J = 12.9, 5.7 Hz, 1H), 1.24 (d, J = 6.9 Hz, 6H) |
| 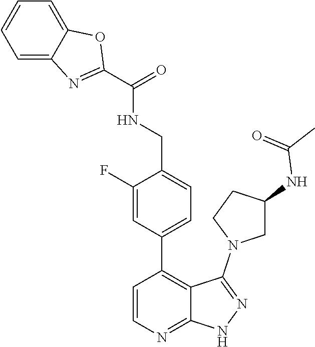 [SMILES: CC(=O)N[C@@H]1CCN(c2n[nH]c3nccc(-c4ccc(CNC(=O)c5nc6ccccc6o5)c(F)c4)c23)C1] | (R)-N-(4-(3-(3- acetamidopyrrolidin- 1-yl)-1H-pyrazolo- [3,4-b]pyridin-4- yl)-2-fluorobenzyl)- benzo[d]oxazole- 2-carboxamide | LCMS (ESI) m/z (M + 1): 514.24 ¹H NMR (400 MHz, DMSO) δ 12.83 (s, 1H), 9.90 (t, J = 6.0 Hz, 1H), 8.46 (d, J = 4.7 Hz, 1H), 7.90 (dd, J = 12.7, 7.8 Hz, 3H), 7.58-7.45 (m, 5H), 7.08 (d, J = 4.7 Hz, 1H), 4.74-4.59 (m, 2H), 4.07 (dd, J = 13.6, 6.6 Hz, 1H), 3.07 (dd, J = 9.9, 6.9 Hz, 1H), 2.85 (dd, J = 16.3, 7.9 Hz, 1H), 2.73 (dd, J = 14.4, 8.6 Hz, 1H), 2.65 (dd, J = 9.9, 5.9 Hz, 1H), 1.91 (dt, J = 14.9, 7.7 Hz, 1H), 1.75 (s, 3H), 1.49 (dt, J = 12.9, 5.5 Hz, 1H) |
| 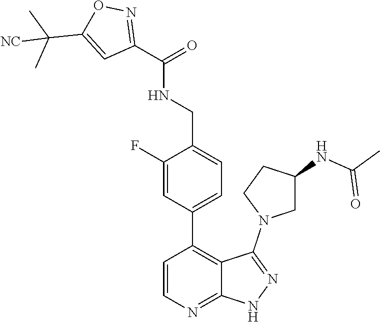 [SMILES: CC(=O)N[C@@H]1CCN(c2n[nH]c3nccc(-c4ccc(CNC(=O)c5cc(C(C)(C)C#N)on5)c(F)c4)c23)C1] | (R)-N-(4-(3-(3- acetamidopyrrolidin- 1-yl)-1H-pyrazolo- [3,4-b]pyridin-4- yl)-2-fluorobenzyl)- 5-(2-cyanopropan- 2-yl)isoxazole-3- carboxamide | LCMS (ESI) m/z (M + 1): 531.28 ¹H NMR (400 MHz, DMSO) δ 12.83 (s, 1H), 9.47 (t, J = 5.9 Hz, 1H), 8.46 (d, J = 4.7 Hz, 1H), 7.90 (d, J = 6.9 Hz, 1H), 7.62-7.40 (m, 3H), 7.07 (d, J = 4.7 Hz, 1H), 7.01 (s, 1H), 4.59 (d, J = 5.8 Hz, 2H), 4.06 (dd, J = 13.3, 6.5 Hz, 1H), 3.07 (dd, J = 10.0, 6.9 Hz, 1H), 2.83 (dd, J = 16.6, 7.8 Hz, 1H), 2.75- 2.63 (m, 2H), 1.89 (dt, J = 14.8, 7.8 Hz, 1H), 1.78 (s, 6H), 1.75 (s, 3H), 1.51-1.44 (m, 1H) |
| 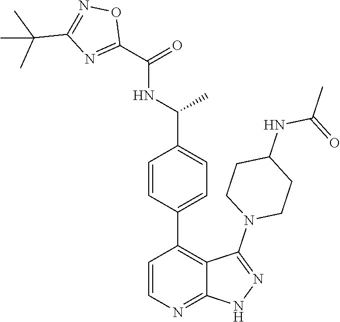 [SMILES: CC(=O)NC1CCN(c2n[nH]c3nccc(-c4ccc([C@@H](C)NC(=O)c5nc(C(C)(C)C)no5)cc4)c23)CC1] | (R)-N-(1-(4-(3-(4- acetamidopiperidin- 1-yl)-1H-pyrazolo- [3,4-b]pyridin-4- yl)phenyl)ethyl)- 3-(tert-butyl)- 1,2,4-oxadiazole- 5-carboxamide | LCMS (ESI) m/z (M + 1): 531.30 ¹H NMR (400 MHz, DMSO) δ 12.84 (s, 1H), 9.88 (d, J = 8.0 Hz, 1H), 8.45 (d, J = 4.6 Hz, 1H), 7.65 (d, J= 8.1 Hz, 2H), 7.57 (d, J = 8.1 Hz, 2H), 7.06 (d, J = 4.7 Hz, 1H), 5.29-5.14 (m, 1H), 3.54-3.42 (m, 2H), 3.06-2.91 (m, 3H), 2.64-2.54 (m, 2H), 1.77 (s, 3H), 1.59 (d, J = 7.0 Hz, 3H), 1.36 (s, 9H), 1.29- 1.11 (m, 2H) |
| 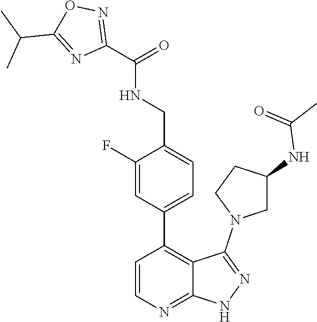 [SMILES: CC(=O)N[C@@H]1CCN(c2n[nH]c3nccc(-c4ccc(CNC(=O)c5noc(C(C)C)n5)c(F)c4)c23)C1] | (R)-N-(4-(3-(3- acetamidopyrrolidin- 1-yl)-1H-pyrazolo- [3,4-b]pyridin-4- yl)-2-fluorobenzyl)- 5-isopropyl-1,2,4- oxadiazole-3- carboxamide | LCMS (ESI) m/z (M + 1): 507.24 ¹H NMR (400 MHz, DMSO) δ 12.85 (s, 1H), 9.57 (s, 1H), 8.46 (d, J = 4.6 Hz, 1H), 7.90 (s, 1H), 7.58-7.41 (m, 3H), 7.08 (d, J = 4.5 Hz, 1H), 4.60 (d, J = 5.3 Hz, 2H), 4.06 (s, 2H), 3.10-2.96 (m, 1H), 2.83 (d, J = 7.0 Hz, 1H), 2.78-2.64 (m, 2H), 1.95-1.84 (m, 1H), 1.75 (s, 3H), 1.54-1.44 (m, 1H), 1.36 (d, J = 6.9 Hz, 6H) |
| 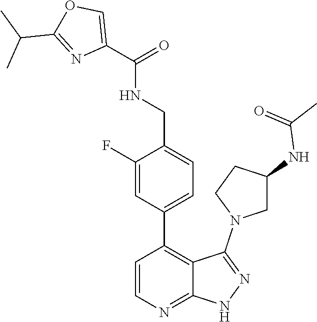 [SMILES: CC(=O)N[C@@H]1CCN(c2n[nH]c3nccc(-c4ccc(CNC(=O)c5coc(C(C)C)n5)c(F)c4)c23)C1] | (R)-N-(4-(3-(3- acetamidopyrrolidin- 1-yl)-1H-pyrazolo- [3,4-b]pyridin-4-yl)- 2-fluorobenzyl)-2- isopropyloxazole- 4-carboxamide | LCMS (ESI) m/z (M + 1): 506.27 ¹H NMR (400 MHz, DMSO) δ 12.84 (s, 1H), 8.77 (t, J = 6.1 Hz, 1H), 8.54 (s, 1H), 8.46 (d, J = 4.7 Hz, 1H), 7.91 (d, J = 6.9 Hz, 1H), 7.62- 7.38 (m, 3H), 7.07 (d, J = 4.7 Hz, 1H), 4.56 (d, J = 6.1 Hz, 2H), 4.06 (dd, J = 13.3, 6.5 Hz, 1H), 3.20- 3.02 (m, 2H), 2.81 (dd, J = 16.6, 7.7 Hz, 1H), 2.74-2.63 (m, 2H), 1.94-1.80 (m, 1H), 1.76 (s, 3H), 1.55-1.38 (m, 1H), 1.30 (d, J = 7.0 Hz, 6H) |
| 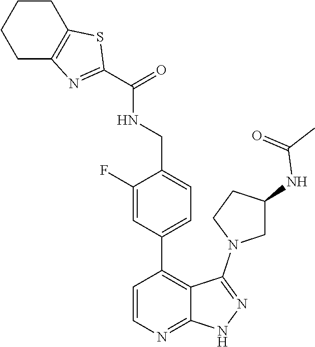 [SMILES: CC(=O)N[C@@H]1CCN(c2n[nH]c3nccc(-c4ccc(CNC(=O)c5nc6c(s5)CCCC6)c(F)c4)c23)C1] | (R)-N-(4-(3-(3- acetamidopyrrolidin- 1-yl)-1H-pyrazolo- [3,4-b]pyridin-4- yl)-2-fluorobenzyl)- 4,5,6,7-tetrahydro- benzo[d]thiazole- 2-carboxamide | LCMS (ESI) m/z (M + 1): 534.23 ¹H NMR (400 MHz, DMSO) δ 12.82 (s, 1H), 9.32 (t, J = 6.1 Hz, 1H), 8.45 (d, J = 4.7 Hz, 1H), 7.88 (d, J = 7.0 Hz, 1H), 7.60-7.36 (m, 3H), 7.07 (d, J = 4.7 Hz, 1H), 4.57 (d, J = 5.9 Hz, 2H), 4.20-3.93 (m, 1H), 3.08 (dd, J = 9.9, 6.9 Hz, 1H), 2.80 (dd, J = 17.0, 9.0 Hz, 3H), 2.75-2.62 (m, 2H), 2.52 (s, 4H), 1.95-1.81 (m, J = 5.2 Hz, 3H), 1.80 (s, 3H), 1.47 (dd, J = 12.5, 7.4 Hz, 1H) |
| 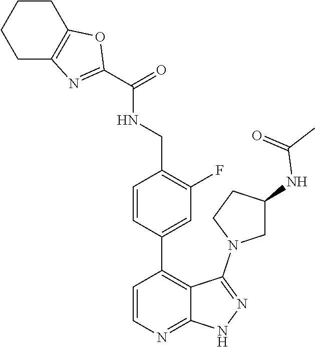 [SMILES: CC(=O)N[C@@H]1CCN(c2n[nH]c3nccc(-c4ccc(CNC(=O)c5nc6c(o5)CCCC6)c(F)c4)c23)C1] | (R)-N-(4-(3-(3- acetamidopyrrolidin- 1-yl)-1H-pyrazolo- [3,4-b]pyridin-4- yl)-2-fluorobenzyl)- 4,5,6,7-tetrahydro- benzo[d]-oxazole- 2-carboxamide | LCMS (ESI) m/z (M + 1): 518.46 ¹H NMR (400 MHz, DMSO) δ 12.83 (s, 1H), 9.41 (t, J = 6.0 Hz, 1H), 8.46 (d, J = 4.7 Hz, 1H), 7.89 (d, J = 6.8 Hz, 1H), 7.63-7.35 (m, 3H), 7.07 (d, J = 4.7 Hz, 1H), 4.56 (d, J = 3.5 Hz, 2H), 4.06 (dd, J = 13.1, 6.7 Hz, 1H), 3.07 (dd, J = 9.9, 6.9 Hz, 1H), 2.83 (dd, J = 16.3, 7.7 Hz, 1H), 2.76-2.61 (m, 5H), 2.01-1.80 (m, 6H), 1.77 (s, 3H), 1.48 (dt, J = 12.8. 6.5 Hz, 1H) |
| 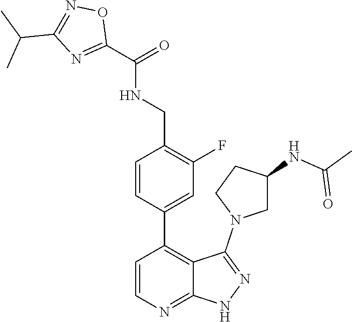 [SMILES: CC(=O)N[C@@H]1CCN(c2n[nH]c3nccc(-c4ccc(CNC(=O)c5nc(C(C)C)no5)c(F)c4)c23)C1] | (R)-N-(4-(3-(3- acetamidopyrrolidin- 1-yl)-1H-pyrazolo- [3,4-b]pyridin-4-yl)- 2-fluorobenzyl)-3- isopropyl-1,2,4- oxadiazole-5- carboxamide | LCMS (ESI) m/z (M + 1): 507.37 ¹H NMR (400 MHz, DMSO) δ 12.83 (s, 1H), 9.94 (t, J = 5.9 Hz, 1H), 8.46 (d, J = 4.7 Hz, 1H), 7.89 (d, J = 6.9 Hz, 1H), 7.51 (ddt, J = 9.4, 7.9, 4.7 Hz, 3H), 7.08 (d, J = 4.7 Hz, 1H), 4.60 (d, J = 5.8 Hz, 2H), 4.05 (dd, J = 13.5, 6.9 Hz, 1H), 3.16 (dt, J = 13.8, 6.9 Hz, 1H), 3.05 (dd, J = 10.0, 6.9 Hz, 1H), 2.85 (dd, J = 16.6, 7.8 Hz, 1H), 2.73 (dd, J = 14.4, 8.7 Hz, 1H), 2.68-2.59 (m, 1H), 1.96-1.84 (m, 1H), 1.75 (s, 3H), 1.48 (dt, J = 13.4, 5.7 Hz, 1H), 1.31 (d, J = 6.9 Hz, 6H) |
| 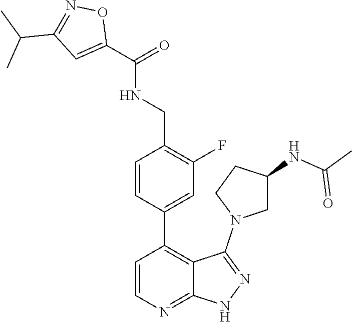 [SMILES: CC(=O)N[C@@H]1CCN(c2n[nH]c3nccc(-c4ccc(CNC(=O)c5cc(C(C)C)no5)c(F)c4)c23)C1] | (R)-N-(4-(3-(3- acetamidopyrrolidin- 1-yl)-1H-pyrazolo- [3,4-b]pyridin-4-yl)- 2-fluorobenzyl)-3- isopropylisoxazole-5- carboxamide | LCMS (ESI) m/z (M + 1): 506.23 ¹H NMR (400 MHz, DMSO) δ 12.83 (s, 1H), 9.49 (t, J = 5.8 Hz, 1H), 8.46 (d, J = 4.7 Hz, 1H), 7.90 (d, J = 7.0 Hz, 1H), 7.59-7.40 (m, 3H), 7.10 (s, 1H), 7.07 (d, J = 4.7 Hz, 1H), 4.58 (d, J = 5.7 Hz, 2H), 4.06 (dd, J = 13.5, 6.7 Hz, 1H), 3.12- 3.00 (m, 2H), 2.84 (dd, J = 16.7, 7.6 Hz, 1H), 2.69 (ddd, J = 15.9, 11.7, 6.8 Hz, 2H), 1.94-1.85 (m, 1H), 1.76 (s, 3H), 1.55-1.43 (m, 1H), 1.24 (d, J = 6.9 Hz, 6H) |
| 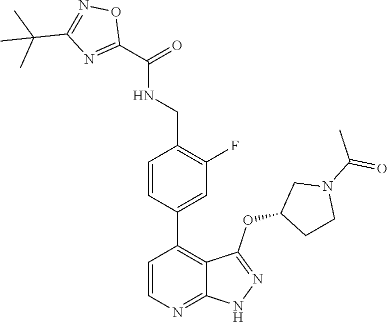 [SMILES: CC(=O)N1CC[C@H](Oc2n[nH]c3nccc(-c4ccc(CNC(=O)c5nc(C(C)(C)C)no5)c(F)c4)c23)C1] | (S)-N-(4-(3-((1- acetylpyrrolidin-3- yl)oxy)-1H-pyrazolo- [3,4-b]pyridin-4-yl)- 2-fluorobenzyl)-3- (tert-butyl)-1,2,4- oxadiazole-5- carboxamide | LCMS (ESI) m/z (M + 1): 522.65 ¹H NMR (400 MHz, DMSO) δ 12.86 (s, 1H), 9.92 (dd, J = 14.7, 6.0 Hz, 1H), 8.52 (dd, J = 4.8, 1.4 Hz, 1H), 7.63-7.42 (m, 3H), 7.18 (dd, J = 6.4, 4.8 Hz, 1H), 5.41-5.34 (m, 1H), 4.58 (d, J = 5.8 Hz, 2H), 3.76- 3.55 (m, 2H), 3.51-3.35 (m, 2H), 2.19-2.02 (m, 2H), 1.88 (d, J = 14.5 Hz, 3H), 1.37 (s, 9H) |

### Example 36: (R)-5-chloro-N-(2-fluoro-4-(3-(pyrrolidin-3-ylamino)-1H-pyrazolo[3,4-b]pyridin-4-yl)benzyl)isoxazole-3-carboxamide

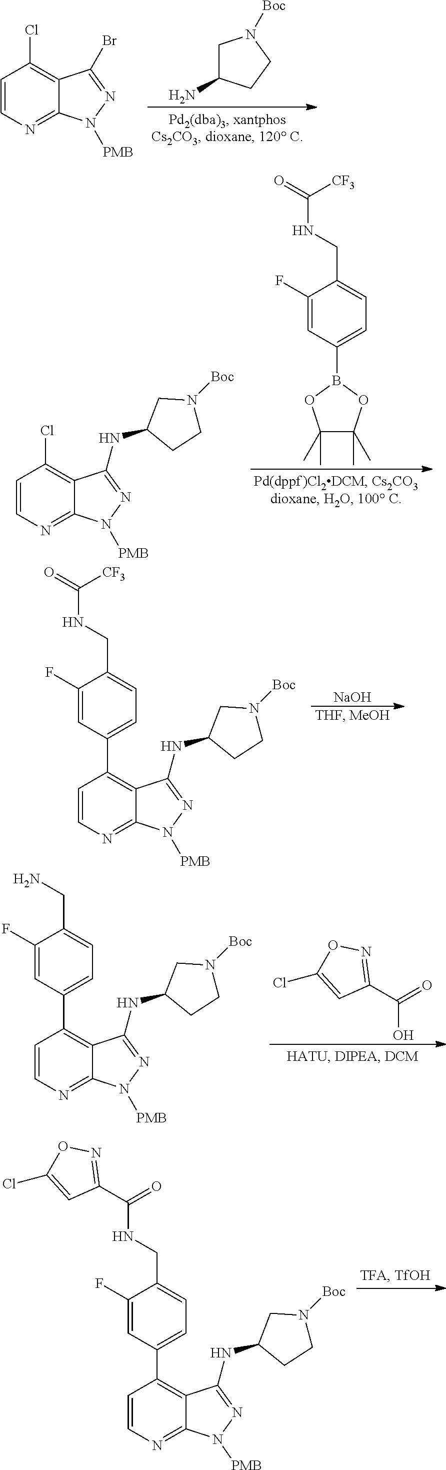

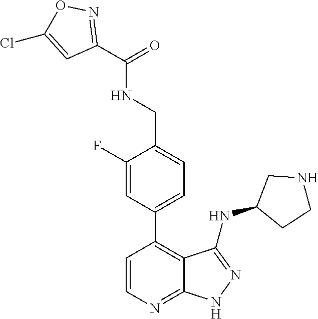 [SMILES: O=C(NCc1ccc(-c2ccnc3[nH]nc(N[C@@H]4CCNC4)c23)cc1F)c1cc(Cl)on1]

At 120° C. under N₂ atmosphere, to a stirred solution of 3-bromo-4-chloro-1-(4-methoxybenzyl)-1H-pyrazolo[3,4-b]pyridine (75 g, 214 mmol) in dioxane (1.5 L) were added tert-butyl (R)-3-aminopyrrolidine-1-carboxylate (51.75 g, 277 mmol), Pd₂(dba)₃ (9.78 g, 10.7 mmol), xantphos (18.6 g, 32.2 mmol) and Cs₂CO₃ (9.3 g, 28.5 mmol). After being stirred at 120° C. for 8 hr, the reaction mixture was cooled down to room temperature and filtered through a pad of celite. The filtration was quenched with H₂O and extracted with ethyl acetate (3×). The combined organic layers were washed with brine and dried over Na₂SO₄. Solvents were removed and the residue was purified by flash chromatography (silica gel, 0˜50% ethyl acetate in petroleum ether) to provide tert-butyl (R)-3-((4-chloro-1-(4-methoxybenzyl)-1H-pyrazolo[3,4-b]pyridin-3-yl)amino)pyrrolidine-1-carboxylate (54 g, 52%) as a yellow oil. LCMS (ESI) m/z (M/M+2): 458.38/460.42.

At 100° C. under N₂ atmosphere, to a stirred solution of tert-butyl (R)-3-((4-chloro-1-(4-methoxybenzyl)-1H-pyrazolo[3,4-b]pyridin-3-yl)amino)pyrrolidine-1-carboxylate (1.0 g, 2.1 mmol) in dioxane/H₂O (50 mL/1 mL) were added 2,2,2-trifluoro-N-(2-fluoro-4-(4,4,5,5-tetramethyl-1,3,2-dioxaborolan-2-yl)benzyl)acetamide (1.13 g, 3.2 mmol), Pd(dppf)Cl₂.DCM (171 mg, 0.21 mmol), and Cs₂CO₃ (1.3 g, 4.2 mmol). After being stirred at 100° C. overnight, the reaction mixture was cooled down to room temperature and filtered through a pad of celite. The filtration was quenched with H₂O and extracted with ethyl acetate (3×). The combined organic layers were washed with brine and dried over Na₂SO₄. Solvents were removed and the residue was purified by flash chromatography (silica gel, 0˜50% ethyl acetate in petroleum ether) to tert-butyl (R)-3-((4-(3-fluoro-4-((2,2,2-trifluoroacetamido)methyl)phenyl)-1-(4-methoxybenzyl)-1H-pyrazolo[3,4-b]pyridin-3-yl)amino)pyrrolidine-1-carboxylate (1.2 g, 89%) as a yellow solid. LCMS (ESI) m/z (M+1): 643.34.

At room temperature, to a stirred solution of tert-butyl (R)-3-((4-(3-fluoro-4-((2,2,2-trifluoroacetamido)methyl)phenyl)-1-(4-methoxybenzyl)-1H-pyrazolo[3,4-b]pyridin-3-yl)amino)pyrrolidine-1-carboxylate (1.2 g, 1.87 mmol) in MeOH (10 mL) was added 1N NaOH (5 mL, 5 mmol). After being stirred at room temperature overnight, the reaction mixture was extracted with ethyl acetate (3×). The combined organic layers were wash with brine and dried over Na₂SO₄. Solvents were removed under vacuum to provide tert-butyl (R)-3-((4-(4-(aminomethyl)-3-fluorophenyl)-1-(4-methoxybenzyl)-1H-pyrazolo[3,4-b]pyridin-3-yl)amino)pyrrolidine-1-carboxylate (1.0 g, quant.) as a yellow solid. LCMS (ESI) m/z (M+1): 547.43.

At 50° C., to a stirred solution of tert-butyl (R)-3-((4-(4-(aminomethyl)-3-fluorophenyl)-1-(4-methoxybenzyl)-1H-pyrazolo[3,4-b]pyridin-3-yl)amino)pyrrolidine-1-carboxylate (350 mg, 0.64 mmol) in DCM (50 mL) were added 5-chloroisoxazole-3-carboxylic acid (113 mg, 0.77 mmol), HATU (487 mg, 1.28 mmol) and DIPEA (248 mg, 1.92 mmol). After being stirred at room temperature overnight, the reaction mixture was quenched with sat. NaHCO₃ and extracted with ethyl acetate (3×). The combined organic layers were washed with brine and dried over Na₂SO₄. Solvents were removed under vacuum to provide tert-butyl (R)-3-((4-(4-((5-chloroisoxazole-3-carboxamido)methyl)-3-fluorophenyl)-1-(4-methoxybenzyl)-1H-pyrazolo[3,4-b]pyridin-3-yl)amino)pyrrolidine-1-carboxylate (268 mg, 62%) as a yellow solid. LCMS (ESI) m/z (M/M+2): 676.19/678.18.

At 0° C., to a stirred solution of tert-butyl (R)-3-((4-(4-((5-chloroisoxazole-3-carboxamido)methyl)-3-fluorophenyl)-1-(4-methoxybenzyl)-1H-pyrazolo[3,4-b]pyridin-3-yl)amino)pyrrolidine-1-carboxylate (100 mg, 0.15 mmol) in TFA (3 mL) was added TfOH (1 mL). After being stirred at room temperature for 4 hr, the reaction mixture was concentrated. The residue was quenched with sat. NaHCO₃ and extracted with DCM (3×). The combined organic layers were washed with brine and dried over Na₂SO₄. Solvents were removed under vacuum and the crude product was purified by prep. HPLC (C18, 0˜90 acetonitrile in H₂O with 0.1% formic acid) to provide (R)-5-chloro-N-(2-fluoro-4-(3-(pyrrolidin-3-ylamino)-1H-pyrazolo[3,4-b]pyridin-4-yl)benzyl)isoxazole-3-carboxamide (40 mg, 59%) as a white solid. LCMS (ESI) m/z (M/M+2): 456.13/458.13. ¹H NMR (400 MHz, DMSO) δ 12.72 (s, 1H), 9.58 (t, J=5.8 Hz, 1H), 9.23 (s, 2H), 8.47 (d, J=4.8 Hz, 1H), 7.65-7.41 (m, 3H), 7.16 (s, 1H), 7.05 (d, J=4.8 Hz, 1H), 4.58 (d, J=5.9 Hz, 2H), 4.28-4.16 (m, 1H), 3.42-3.30 (m, 1H), 3.29-3.13 (m, 3H), 2.24-2.10 (m, 1H), 1.90-1.77 (m, 1H).

The following compounds were prepared by analogous methods:

| Chemical structure | Chemical names | LC-MS/HNMR |
|---|---|---|
| 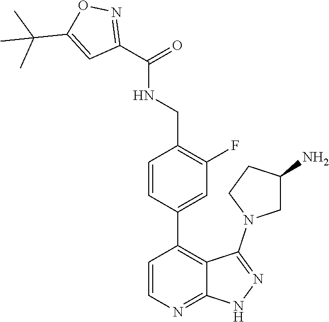 [SMILES: CC(C)(C)c1cc(C(=O)NCc2ccc(-c3ccnc4[nH]nc(N5CC[C@@H](N)C5)c34)cc2F)no1] | (R)-N-(4-(3-(3- aminopyrrolidin-1- yl)-1H-pyrazolo- [3,4-b]pyridin-4-yl)- 2-fluorobenzyl)-5- (tert-butyl)isoxazole- 3-carboxamide | LCMS (ESI) m/z (M + 1): 478.35 ¹H NMR (400 MHz. DMSO) δ 12.97 (s, 1H), 9.36 (t, J = 6.0 Hz, 1H), 8.49 (d, J = 4.7 Hz, 1H), 8.12 (s, 3H), 7.66-7.43 (m, 3H), 7.12 (d, J = 4.7 Hz, 1H), 6.63 (s, 1H), 4.57 (d, J = 5.9 Hz, 2H), 3.66 (s, 1H), 3.28 (dd, J - 10.9, 7.2 Hz, 1H), 2.98 (dd, J = 10.9, 5.4 Hz, 1H), 2.82 (dd, J = 16.7, 7.3 Hz, 1H), 2.67 (dd, J = 14.0, 8.6 Hz, 1H), 1.97 (dt, J = 15.2, 7.7 Hz, 1H), 1.66 (dt, J = 12.8, 5.3 Hz, 1H), 1.33 (s, 9H) |
| 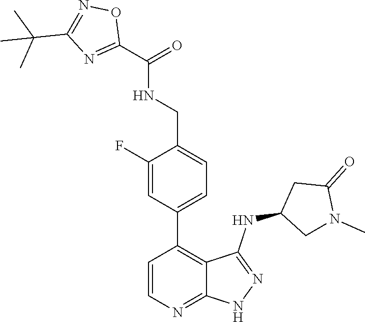 [SMILES: CN1C[C@@H](Nc2n[nH]c3nccc(-c4ccc(CNC(=O)c5nc(C(C)(C)C)no5)c(F)c4)c23)CC1=O] | (S)-3-(tert-butyl)-N- (2-fluoro-4-(3-((1- methyl-5- oxopyrrolidin-3-yl)- amino)-1H-pyrazolo- [3,4-b]pyridin-4- yl)benzyl)-1,2,4- oxadiazole-5- carboxamide | LCMS (ESI) m/z (M + 1): 507.26 ¹H NMR (400 MHz, DMSO) δ 12.59 (s, 1H), 9.93 (t, J = 6.0 Hz, 1H), 8.44 (d, J = 4.7 Hz, 1H), 7.56 (t, J = 7.9 Hz, 1H), 7.51 (dd, J = 11.0, 1.4 Hz, 1H), 7.45 (dd, J = 25 Spectral Size 65536 7.9, 1.6 Hz, 1H), 7.00 (d, J = 4.8 Hz, 1H), 4.84 (d, J = 6.7 Hz, 1H), 4.60 (d, J = 5.7 Hz, 2H), 4.23 (d, J = 6.8 Hz, 1H), 3.68 (dd, J = 10.0, 7.4 Hz, 1H), 3.14 (dd, J = 10.1, 4.4 Hz, 1H), 2.68 (s, 3H), 2.62 (dd, J = 17.0, 8.4 Hz, 1H), 2.11 (dd, J = 17.0, 5.3 Hz, 1H), 1.37 (s, 9H) |
| 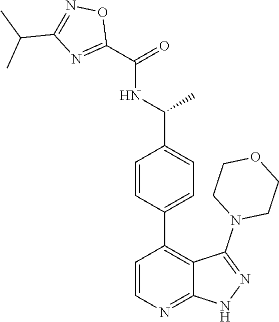 [SMILES: CC(C)c1noc(C(=O)N[C@H](C)c2ccc(-c3ccnc4[nH]nc(N5CCOCC5)c34)cc2)n1] | (R)-3-isopropyl-N-(1- (4-(3-morpholino-1H- pyrazolo[3,4-b]pyridin- 4-yl)phenyl)ethyl)- 1,2,4-oxadiazole-5- carboxamide | LCMS (ESI) m/z (M+ 1): 462.32 ¹H NMR (400 MHz, DMSO) δ 12.92 (s, 1H), 9.93 (d, J = 8.1 Hz, 1H), 8.48 (d, J = 4.7 Hz, 1H), 7.63 (dd, J = 27.1, 8.3 Hz, 4H), 7.09 (d, J = 4.7 Hz, 1H), 5.33-5.01 (m, 1H), 3.39-3.34 (m, 4H), 3.17 (dt, J = 13.9, 6.9 Hz, 1H), 2.73-2.64 (m, 4H), 1.57 (d, J = 7.1 Hz, 3H), 1.32 (d, J = 6.9 Hz, 6H) |
| 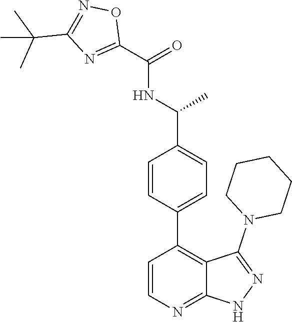 [SMILES: C[C@@H](NC(=O)c1nc(C(C)(C)C)no1)c1ccc(-c2ccnc3[nH]nc(N4CCCCC4)c23)cc1] | (R)-3-(tert-butyl)-N- (1-(4-(3-(piperidin- 1-yl)-1H-pyrazolo- [3,4-b]pyridin-4- yl)phenyl)ethyl)- 1,2,4-oxadiazole- 5-carboxamide | LCMS (ESI) m/z (M + 1): 474.39 ¹H NMR (400 MHz, DMSO) δ 12.79 (s, 1H), 9.87 (d, J = 8.1 Hz, 1H), 8.45 (d, J = 4.7 Hz. 1H), 7.62 (dd, J = 24.5, 8.2 Hz, 4H), 7.05 (d, J = 4.7 Hz, 1H), 5.32-5.09 (m, 1H), 2.68 (s, 4H), 1.57 (d, J = 7.1 Hz, 3H), 1.35 (s, 9H), 1.25-1.18 (m, 6H) |
| 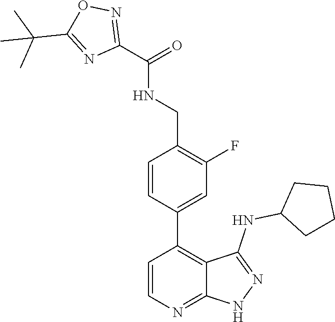 [SMILES: CC(C)(C)c1nc(C(=O)NCc2ccc(-c3ccnc4[nH]nc(NC5CCCC5)c34)cc2F)no1] | 5-(tert-butyl)-N-(4-(3- (cyclopentylamino)-1H- pyrazolo[3,4-b]pyridin- 4-yl)-2-fluorobenzyl)- 1,2,4-oxadiazole-3- carboxamide | LCMS (ESI) m/z (M + 1): 478.58 ¹H NMR (400 MHz, DMSO) δ 12.41 (s, 1H), 9.54 (t, J = 6.0 Hz, 1H), 8.41 (d, J = 4.7 Hz, 1H), 7.65-7.37 (m, 3H), 6.95 (d, J = 4.7 Hz, 1H), 4.59 (d, J = 6.0 Hz, 2H), 4.03-3.81 (m, 2H), 1.91-1.71 (m, 2H), 1.58- 1.48 (m, 3H), 1.48-1.39 (m, 9H), 1.38-1.30 (m, 2H) |
| 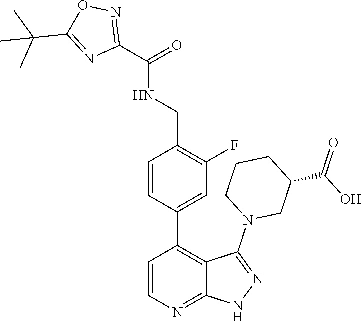 [SMILES: CC(C)(C)c1nc(C(=O)NCc2ccc(-c3ccnc4[nH]nc(N5CCC[C@H](C(=O)O)C5)c34)cc2F)no1] | (S)-1-(4-(4-((5-(tert- butyl)-1,2,4-oxadiazole- 3-carboxamido)methyl)- 3-fluorophenyl)-1H- pyrazolo[3,4-b]pyridin- 3-yl)piperidine-3- carboxylic acid | LCMS (ESI) m/z (M + 1): 522.36 ¹H NMR (400 MHz, DMSO) δ 12.95 (s, 1H), 12.22 (s, 1H), 9.50 (t, J = 6.0 Hz, 1H), 8.49 (d, J = 4.7 Hz, 1H), 7.62-7.40 (m, 3H), 7.12 (d, J = 4.7 Hz, 1H), 4.57 (d, J = 5.6 Hz, 2H), 3.31-3.24 (m, 1H), 2.90- 2.69 (m, 2H), 2.41-2.25 (m, 2H), 1.88-1.69 (m, 1H), 1.43 (s, 9H), 1.38-1.24 (m, 2H), 1.18-1.07 (m, 1H) |
| 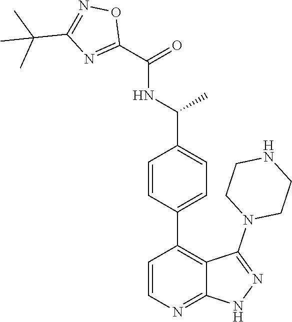 [SMILES: C[C@@H](NC(=O)c1nc(C(C)(C)C)no1)c1ccc(-c2ccnc3[nH]nc(N4CCNCC4)c23)cc1] | (R)-3-(tert-butyl)-N- (1-(4-(3-(piperazin-1- yl)-1H-pyrazolo- [3,4-b]-pyridin-4- yl)phenyl)ethyl)- 1,2,4-oxadiazole-5- carboxamide | LCMS (ESI) m/z (M + 1): 475.66 ¹H NMR (400 MHz. DMSO) δ 13.09 (s, 1H), 9.90 (d. J = 7.8 Hz, 1H), 8.95 (s, 3H), 8.51 (d, J = 4.7 Hz, 1H), 7.75-7.45 (m, 4H), 7.12 (d, J = 4.7 Hz, 1H), 5.35-5.07 (m, 1H), 3.03-2.92 (m, 4H), 2.92- 2.82 (m, 4H), 1.59 (d, J = 7.0 Hz, 3H), 1.37 (s, 9H) |
| 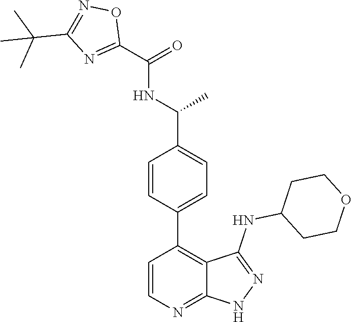 [SMILES: C[C@@H](NC(=O)c1nc(C(C)(C)C)no1)c1ccc(-c2ccnc3[nH]nc(NC4CCOCC4)c23)cc1] | (R)-3-(tert-butyl)-N- (1-(4-(3-((tetrahydro- 2H-pyran-4-yl)- amino)-1H-pyrazolo- [3,4-b]pyridin-4- yl)phenyl)ethyl)- 1,2,4-oxadiazole- 5-carboxamide | LCMS (ESI) m/z (M + 1): 490.56 ¹H NMR (400 MHz, DMSO) δ 12.40 (s, 1H), 9.89 (d, J = 8.1 Hz, 1H), 8.41 (d, J = 4.7 Hz, 1H), 7.68-7.51 (m, 3H), 6.93 (d, J = 4.7 Hz, 1H), 5.36-5.11 (m, 1H), 3.86 (d, J = 7.2 Hz, 1H), 3.70-3.62 (m, 2H), 3.56-3.46 (m, 1H), 3.30-3.21 (m, 1H), 1.82 (d, J = 9.4 Hz, 2H), 1.58 (d, J = 7.1 Hz, 3H), 1.37 (s, 9H), 1.29-1.20 (m, 2H) |
| 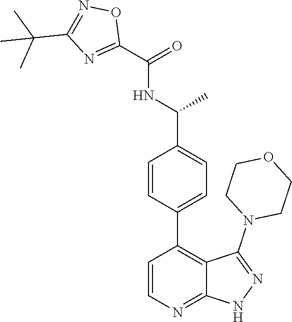 [SMILES: C[C@@H](NC(=O)c1nc(C(C)(C)C)no1)c1ccc(-c2ccnc3[nH]nc(N4CCOCC4)c23)cc1] | (R)-3-(tert-butyl)-N- (1-(4-(3-morpholino- 1H-pyrazolo[3,4-b]- pyridin-4-yl)- phenyl)ethyl)- 1,2,4-oxadiazole- 5-carboxamide | LCMS (ESI) m/z (M + 1): 476.40 ¹H NMR (400 MHz, DMSO) δ 12.93 (s, 1H), 9.88 (d, J = 7.9 Hz, 1H), 8.48 (d, J = 4.7 Hz, 1H), 7.63 (dd, J = 27.4, 8.2 Hz, 4H), 7.09 (d, J = 4.7 Hz, 1H), 5.27-5.17 (m, 1H), 3.41-3.33 (m, 4H), 2.75-2.62 (m, 4H), 1.57 (d, J = 7.0 Hz, 3H), 1.37 (s, 9H) |
| 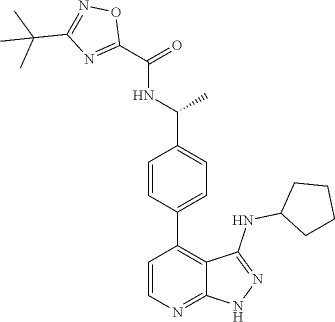 [SMILES: C[C@@H](NC(=O)c1nc(C(C)(C)C)no1)c1ccc(-c2ccnc3[nH]nc(NC4CCCC4)c23)cc1] | (R)-3-(tert-butyl)-N- (1-(4-(3-(cyclopentyl- amino)-1H-pyrazolo- [3,4-b]pyridin-4-yl)- phenyl)ethyl)-1,2,4- oxadiazole-5- carboxamide | LCMS (ESI) m/z (M + 1): 474.39 ¹H NMR (400 MHz, DMSO) δ 12.33 (s, 1H), 9.89 (d, J = 8.1 Hz, 1H), 8.39 (d, J = 4.7 Hz, 1H), 7.58 (dd, J = 28.1, 8.2 Hz, 4H), 6.91 (d, J = 4.7 Hz, 1H), 5.39-5.12 (m, 1H), 3.86 (dt, J = 11.6, 5.8 Hz, 1H), 3.69 (d, J = 6.7 Hz, 1H), 1.77 (dt, J = 13.5, 6.6 Hz, 2H), 1.58 (d, J = 7.1 Hz, 3H), 1.47-1.40 (m, 4H), 1.37 (s, 9H), 1.31-1.21 (m, 2H) |
| 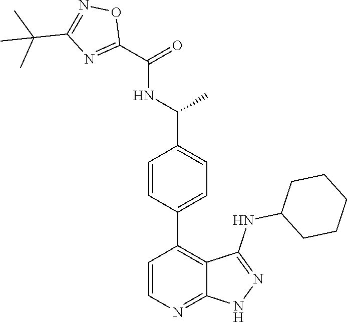 [SMILES: C[C@@H](NC(=O)c1nc(C(C)(C)C)no1)c1ccc(-c2ccnc3[nH]nc(NC4CCCCC4)c23)cc1] | (R)-3-(tert-butyl)-N- (1-(4-(3-(cyclo- hexylamino)-1H- pyrazolo[3,4-b]- pyridin-4-yl)phenyl)- ethyl)-1,2,4- oxadiazole-5- carboxamide | LCMS (ESI) m/z (M + 1): 488.46 ¹H NMR (400 MHz, DMSO) δ 12.31 (s, 1H), 9.89 (d, J = 8.1 Hz, 1H), 8.39 (d, J = 4.7 Hz, 1H), 7.59 (dd, J = 9.1, 8.2 Hz, 4H), 6.91 (d, J = 4.7 Hz, 1H), 5.33-5.05 (m, 1H), 3.68 (d, J = 7.6 Hz, 1H), 3.46-3.35 (m, 1H), 3.31-3.28 (m, 1H), 1.75 (d, J = 11.9 Hz, 2H), 1.58 (d, J = 7.1 Hz, 3H), 1.43-1.38 (m, 2H), 1.37 (s, 9H), 1.22-1.03 (m. 5H) |
| 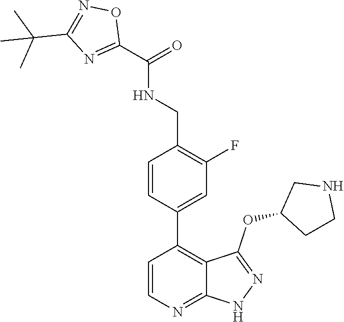 [SMILES: CC(C)(C)c1noc(C(=O)NCc2ccc(-c3ccnc4[nH]nc(O[C@H]5CCNC5)c34)cc2F)n1] | (S)-3-(tert-butyl)-N- (2-fluoro-4-(3- (pyrrolidin-3-yloxy)- 1H-pyrazolo[3,4-b]- pyridin-4-yl)benzyl)- 1,2,4-oxadiazole-5- carboxamide | LCMS (ESI) m/z (M + 1): 480.62 ¹H NMR (400 MHz, DMSO) δ 12.95 (s, 1H), 9.95 (t, J = 5.9 Hz, 1H), 9.42 (d, J = 18.6 Hz, 2H), 8.55 (d, J = 4.8 Hz, 1H), 7.71-7.64 (m, 1H), 7.63-7.52 (m, 2H), 7.21 (d, J = 4.8 Hz, 1H), 5.39 (s, 1H), 4.59 (d, J = 5.9 Hz, 2H), 3.60-3.47 (m, 2H), 3.39-3.30 (m, 1H), 3.25-3.13 (m, 1H), 2.25-2.03 (m, 2H), 1.37 (s, 9H) |

### Example 37: (R)-5-chloro-N-(4-(3-((1-(dimethylcarbamoyl)pyrrolidin-3-yl)amino)-1H-pyrazolo[3,4-b]pyridin-4-yl)-2-fluorobenzyl)isoxaz-3-carboxamide

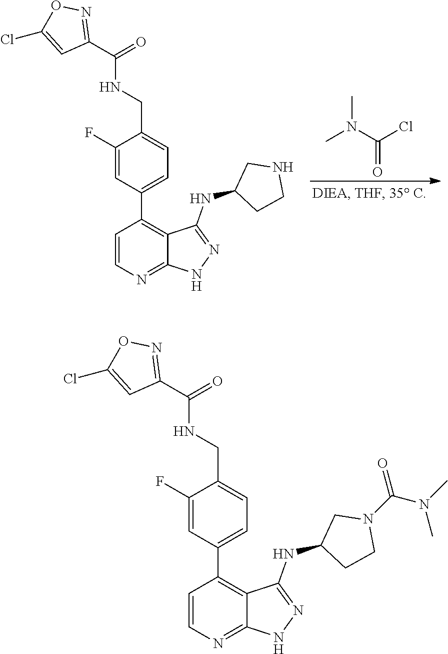

At 35° C., to a stirred solution of (R)-5-chloro-N-(2-fluoro-4-(3-(pyrrolidin-3-ylamino)-1H-pyrazolo[3,4-b]pyridin-4-yl)benzyl)isoxazole-3-carboxamide (130 mg, 0.29 mmol) in THF (10 mL) was added DIEA (112 mg, 0.87 mmol) followed by dimethylcarbamic chloride (62 mg, 0.58 mmol, in 1 mL THF). After being stirred at room temperature for 1 hr, the reaction mixture was quenched with sat. NaHCO₃ and extracted with DCM (3×). The combined organic layers were washed with brine and dried over Na₂SO₄. Solvents were removed under vacuum and the residue was purified by flash chromatography (silica gel, 0˜10% MeOH in DCM) to provide (R)-5-chloro-N-(4-(3-((1-(dimethylcarbamoyl)pyrrolidin-3-yl)amino)-1H-pyrazolo[3,4-b]pyridin-4-yl)-2-fluorobenzyl)isoxazole-3-carboxamide (11 mg, 7%) as a yellow solid. LCMS (ESI) m/z (M+1): 527.46/529.44. ¹H NMR (400 MHz, DMSO) δ 12.54 (s, 1H), 9.53 (t, J=5.9 Hz, 1H), 8.43 (d, J=4.7 Hz, 1H), 7.56-7.47 (m, 2H), 7.42 (dd, J=7.9, 1.5 Hz, 1H), 7.12 (s, 1H), 6.99 (d, J=4.7 Hz, 1H), 4.57 (d, J=5.9 Hz, 2H), 4.37 (d, J=5.5 Hz, 1H), 4.07 (dd, J=10.1, 5.1 Hz, 1H), 3.51 (dd, J=10.7, 5.6 Hz, 1H), 3.42-3.26 (m, 2H), 3.20 (dd, J=10.6, 4.2 Hz, 1H), 2.69 (s, 6H), 2.16-1.93 (m, 1H), 1.83-1.63 (m, 1H).

The following compounds were prepared by analogous methods:

| Chemical structure | Chemical names | LC-MS/HNMR |
|---|---|---|
| 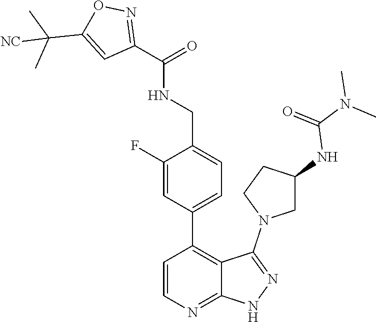 [SMILES: CN(C)C(=O)N[C@@H]1CCN(c2n[nH]c3nccc(-c4ccc(CNC(=O)c5cc(C(C)(C)C#N)on5)c(F)c4)c23)C1] | (R)-5-(2-cyanopropan-2- yl)-N-(4-(3-(3-(3,3- dimethylureido)pyrrolidin- 1-yl)-1H-pyrazolo- [3,4-b]pyridin-4-yl)-2- fluorobenzyl)isoxazole- 3-carboxamide | LCMS (ESI) m/z (M + 1): 560.29 ¹H NMR (400 MHz, DMSO) δ 12.80 (s, 1H), 9.47 (t, J = 5.9 Hz, 1H), 8.46 (d, J = 4.7 Hz, 1H), 7.57-7.42 (m, 3H), 7.07 (d, J = 4.7 Hz, 1H), 7.01 (s, 1H), 6.05 (d, J = 6.8 Hz, 1H), 4.65-4.51 (m, 2H), 4.01 (dq, J = 13.4, 6.7 Hz, 1H), 3.08 (dd, J = 9.8, 6.9 Hz, 1H), 2.91-2.77 (m, 2H), 2.73 (s, 6H), 2.71-2.62 (m, 1H), 1.91-1.82 (m, 1H), 1.78 (s, 6H), 1.61-1.48 (m, 1H) |
| 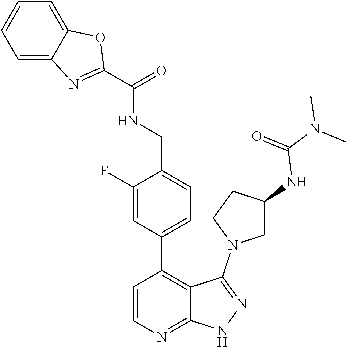 [SMILES: CN(C)C(=O)N[C@@H]1CCN(c2n[nH]c3nccc(-c4ccc(CNC(=O)c5nc6ccccc6o5)c(F)c4)c23)C1] | (R)-N-(4-(3-(3-(3- dimethylureido)pyrrolidin- 1-yl)-1H-pyrazolo- [3,4-b]pyridin-4-yl)-2- fluorobenzyl)benzo[d]- oxazole-2-carboxamide | LCMS (ESI) m/z (M + 1): 543.30 ¹H NMR (400 MHz, DMSO) δ 12.80 (s, 1H), 9.89 (t, J = 6.0 Hz, 1H), 8.46 (d, J = 4.7 Hz, 1H), 7.90 (dd, J = 14.5, 7.8 Hz, 2H), 7.52 (dddd, J = 14.8, 11.3, 7.7, 1.4 Hz, 5H), 7.07 (d, J = 4.7 Hz, 1H), 6.04 (d, J = 6.8 Hz, 1H), 4.65 (d, J = 5.3 Hz, 2H), 4.01 (dt, J = 13.2, 6.5 Hz, 1H), 3.09 (dd, J = 9.9, 6.9 Hz, 1H), 2.83 (d, J = 8.1 Hz, 1H), 2.72 (s, 6H), 2.69-2.63 (m, 1H), 1.87 (dt, J = 20.2, 7.6 Hz, 1H), 1.55 (dt, J = 13.7, 6.0 Hz, 1H) |
| 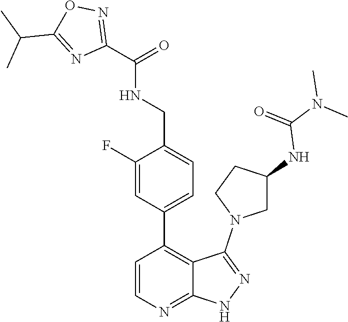 [SMILES: CC(C)c1nc(C(=O)NCc2ccc(-c3ccnc4[nH]nc(N5CC[C@@H](NC(=O)N(C)C)C5)c34)cc2F)no1] | (R)-N-(4-(3-(3-(3,3- dimethylureido)pyrrolidin- 1-yl)-1H-pyrazolo- [3,4-b]pyridin-4-yl)-2- fluorobenzyl)-5-isopropyl- 1,2,4-oxadiazole-3- carboxamide | LCMS (ESI) m/z (M + 1): 536.27 ¹H NMR (400 MHz, DMSO) δ 12.80 (s, 1H), 9.55 (t, J = 5.9 Hz, 1H), 8.46 (d, J = 4.7 Hz, 1H), 7.66-7.35 (m, 3H), 7.07 (d, J = 4.7 Hz, 1H), 6.04 (d, J = 6.9 Hz, 1H), 4.73-4.48 (m, 2H), 4.12-3.91 (m, 2H), 3.40- 3.34 (m, 1H), 3.07 (dd, J = 9.9, 6.8 Hz, 1H), 2.87-2.79 (m, 1H), 2.73 (s, 6H), 2.69-2.62 (m, 1H), 1.85 (dd, J = 13.0, 7.1 Hz, 1H), 1.54 (d, J = 5.8 Hz, 1H), 1.36 (d, J = 7.0 Hz, 6H) |
| 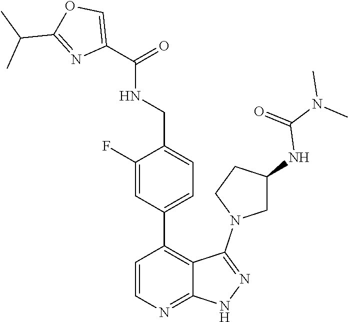 [SMILES: CC(C)c1nc(C(=O)NCc2ccc(-c3ccnc4[nH]nc(N5CC[C@@H](NC(=O)N(C)C)C5)c34)cc2F)co1] | (R)-N-(4-(3-(3-(3,3- dimethylureido)pyrrolidin- 1-yl)-1H-pyrazolo- [3,4-b]pyridin-4-yl)-2- fluorobenzyl)-2- isopropyloxazole-4- carboxamide | LCMS (ESI) m/z (M + 1): 535.29 ¹H NMR (400 MHz, DMSO) δ 12.79 (s, 1H), 8.74 (t, J = 6.1 Hz, 1H), 8.53 (s, 1H), 8.45 (d, J = 4.7 Hz, 1H), 7.56-7.38 (m, 3H), 7.06 (d, J = 4.7 Hz, 1H), 6.04 (d, J = 6.8 Hz, 1H), 4.65-4.47 (m, 2H), 4.02 (dd, J = 13.9, 6.8 Hz, 1H), 3.18- 3.03 (m, 2H), 2.87-2.77 (m, 1H), 2.75 (s, 6H), 2.70-2.60 (m, 1H), 1.90-1.78 (m, 1H), 1.58-1.46 (m, 1H), 1.30 (d, J = 7.0 Hz, 6H) |
| 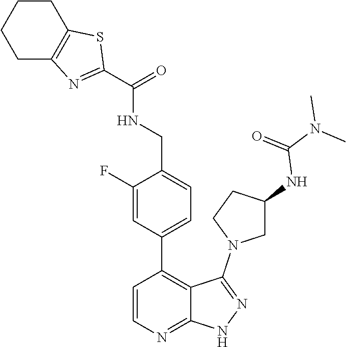 [SMILES: CN(C)C(=O)N[C@@H]1CCN(c2n[nH]c3nccc(-c4ccc(CNC(=O)c5nc6c(s5)CCCC6)c(F)c4)c23)C1] | (R)-N-(4-(3-(3-(3,3- dimethylureido)pyrrolidin- 1-yl)-1H-pyrazolo- [3,4-b]pyridin-4-yl)-2- fluorobenzyl)-4,5,6,7- tetrahydrobenzo[d]- thiazole-2-carboxamide | LCMS (ESI) M/Z (M + 1): 563.26 ¹H NMR (400 MHZ, DMSO) Δ 12.79 (S, 1H), 9.31 (T, J = 6.1 HZ, 1H), 8.45 (D, J = 4.7 HZ, 1H), 7.54-7.41 (M, 3H), 7.06 (D, J = 4.7 HZ, 1H), 6.03 (D, J = 6.9 HZ, 1H), 4.57 (D, J = 6.0 HZ, 2H), 4.02 (DD, J = 13.6, 7.1 HZ, 1H), 3.10 (DD, J = 9.9, 6.9 HZ, 1H), 2.88-2.77 (M, 2H), 2.74 (S, 6H), 2.64 (DD, J = 14.4, 8.1 HZ, 1H), 2.60-2.51 (M, 2H), 1.92-1.76 (M, 4H), 1.59- 1.47 (M, 1H) |
| 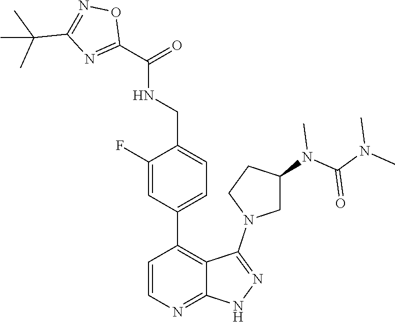 [SMILES: CN(C)C(=O)N(C)[C@@H]1CCN(c2n[nH]c3nccc(-c4ccc(CNC(=O)c5nc(C(C)(C)C)no5)c(F)c4)c23)C1] | (R)-3-(tert-butyl)-N- (2-fluoro-4-(3-(3-(1,3,3- trimethylureido)pyrrolidin- 1-yl)-1H-pyrazolo- [3,4-b]pyridin-4-yl)- benzyl)-1,2,4-oxadiazole- 5-carboxamide | LCMS (ESI) M/Z (M + 1): 564.31 ¹H NMR (400 MHZ, DMSO) Δ 12.87 (S, 1H), 9.93 (T, J = 6.0 HZ, 1H), 8.97-8.17 (M, 1H), 7.67-7.39 (M, 2H), 7.08 (D, J = 4.7 HZ, 1H), 4.59 (D, J = 6.0 HZ, 2H), 4.20 (DD, J = 13.7, 7.4 HZ, 1H), 3.15 (DD, J = 10.4, 8.3 HZ, 1H), 2.84 (DD, J = 10.5, 5.3 HZ, 1H), 2.68 (S, J = 12.6 HZ, 6H), 2.56 (D, J = 7.4 HZ, 1H), 1.87-1.79 (M, 1H), 1.64- 1.52 (M, 1H), 1.36 (S, 9H) |
| 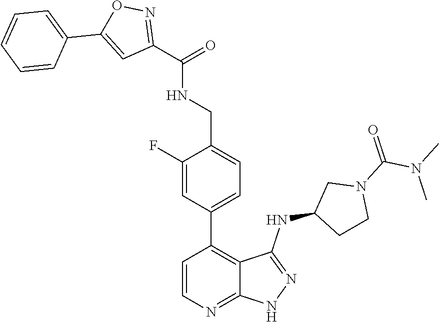 [SMILES: CN(C)C(=O)N1CC[C@@H](Nc2n[nH]c3nccc(-c4ccc(CNC(=O)c5cc(-c6ccccc6)on5)c(F)c4)c23)C1] | (R)-N-(4-(3-((1- (dimethylcarbamoyl)- pyrrolidin-3-yl)amino)- 1H-pyrazolo[3,4-b]- pyridin-4-yl)-2- fluorobenzyl)-5- phenylisoxazole-3- carboxamide | LCMS (ESI) M/Z (M + 1): 569.54 ¹H NMR (400 MHZ, DMSO) Δ 12.53 (S, 1H), 9.47 (T, J = 5.9 HZ, 1H), 8.43 (D, J = 4.7 HZ, 1H), 7.95 (DD, J = 7.5, 2.0 HZ, 2H), 7.66-7.43 (M, 6H), 7.45 (S, 1H), 6.99 (D, J = 4.8 HZ, 1H), 4.61 (D, J = 5.9 HZ, 2H), 4.38 (D, J = 5.5 HZ, 1H), 4.07 (DD, J = 10.1, 5.1 HZ, 1H), 3.51 (DD, J = 10.7, 5.6 HZ, 1H), 3.32-3.09 (M, 2H), 3.21 (DD, J = 10.6, 4.2 HZ, 1H), 2.68 (S, 6H), 2.24-1.93 (M, 1H), 1.81-1.61 (M, 1H) |
| 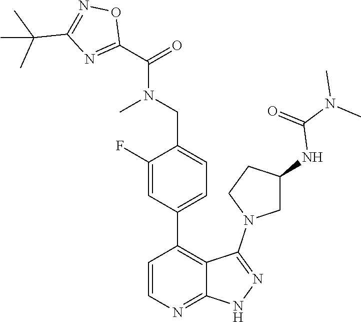 [SMILES: CN(C)C(=O)N[C@@H]1CCN(c2n[nH]c3nccc(-c4ccc(CN(C)C(=O)c5nc(C(C)(C)C)no5)c(F)c4)c23)C1] | (R)-3-(tert-butyl)-n-(4- (3-(3-(3,3-dimethyl- ureido)pyrrolidin-1- yl)-1H-pyrazolo- [3,4-b]pyridin-4-yl)-2- fluorobenzyl)-n-methyl- 1,2,4-oxadiazole-5- carboxamide | LCMS (ESI) M/Z (M + 1): 564.27 ¹H NMR (400 MHZ, DMSO) Δ 12.82 (S, 1H), 8.47 (DD, J = 4.7, 3.6 HZ, 1H), 7.64-7.45 (M, 3H), 7.08 (DD, J = 11.5, 4.7 HZ, 1H), 6.04 (D, J = 6.7 HZ, 1H), 4.96 (Q, J = 16.1 HZ, 1H), 4.86 (S, 1H), 4.01 (DD, J = 13.1, 8.0 HZ, 1H), 3.18-3.01 (M, 4H), 2.91-2.63 (M, 9H), 1.92-1.79 (M, 1H), 1.59-1.49 (M, 1H), 1.34 (D, J = 15.9 HZ, 9H) |
| 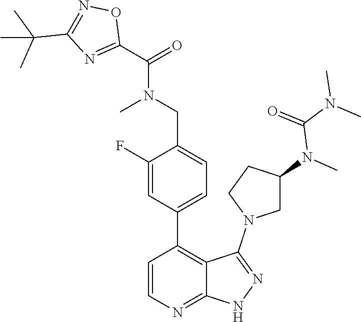 [SMILES: CN(C)C(=O)N(C)[C@@H]1CCN(c2n[nH]c3nccc(-c4ccc(CN(C)C(=O)c5nc(C(C)(C)C)no5)c(F)c4)c23)C1] | (R)-3-(tert-butyl)-n-(2- fluoro-4-(3-(3-(1,3,3- trimethylureido)- pyrrolidin-1-yl)-1H- pyrazolo[3,4-b]pyridin- 4-yl)benzyl)-n-methyl- 1,2,4-oxadiazole-5- carboxamide | LCMS (ESI) M/Z (M + l): 578.31 ¹H NMR (400 MHZ, DMSO) Δ 12.89 (S, ¹H), 8.48 (DD, J = 4.7, 2.7 HZ, 1H), 7.64-7.45 (M, 3H), 7.10 (DD, J = 8.3, 4.7 HZ, 1H), 5.00-4.83 (M, 2H), 4.26-4.18 (M, 1H), 3.23-3.03 (M, 4H), 2.90-2.83 (M, 1H), 2.75- 2.51 (M, 11H), 1.88-1.79 (M, 1H), 1.62-1.53 (M, 1H), 1.34 (D, J = 14.6 HZ, 9H) |
| 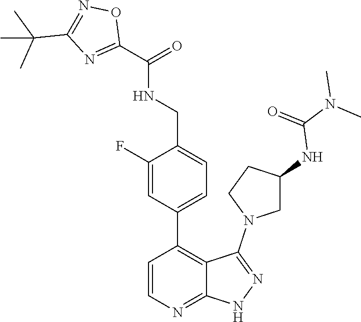 [SMILES: CN(C)C(=O)N[C@@H]1CCN(c2n[nH]c3nccc(-c4ccc(CNC(=O)c5nc(C(C)(C)C)no5)c(F)c4)c23)C1] | (R)-3-(tert-butyl)-n- (4-(3-(3-(3,3-dimethyl- ureido)pyrrolidin-1- yl)-1H-pyrazolo- [3,4-b]pyridin-4-yl)- 2-fluorobenzyl)- 1,2,4-oxadiazole-5- carboxamide | LCMS (ESI) M/Z (M + 1): 550.35 ¹H NMR (400 MHZ, DMSO) Δ 12.80 (S, 1H), 9.93 (T, J = 5.8 HZ, 1H), 8.46 (D, J = 4.7 HZ, 1H), 7.60-7.45 (M, 3H), 7.07 (D, J = 4.7 HZ, 1H), 6.05 (D, J = 6.9 HZ, 1H), 4.61 (DDD, J = 20.5, 15.2, 5.9 HZ, 2H), 4.00 (DD, J = 14.6, 6.8 HZ, 1H), 3.06 (DD, J = 9.7, 6.9 HZ, 1H), 2.85 (DD, J = 16.2, 8.2 HZ, 1H), 2.80-2.59 (M, 8H), 1.90-1.81 (M, 1H), 1.59-1.51 (M, 1H), 1.36 (S, 9H) |
| 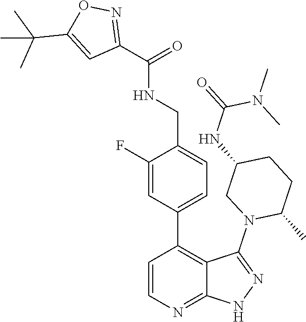 [SMILES: C[C@H]1CC[C@@H](NC(=O)N(C)C)CN1c1n[nH]c2nccc(-c3ccc(CNC(=O)c4cc(C(C)(C)C)on4)c(F)c3)c12] | 5-(tert-butyl)-N-(4-(3- ((2S,5R)-5-(3,3- dimethylureido)-2- methylpiperidin-1-yl)- 1H-pyrazolo[3,4-b]- pyridin-4-yl)-2- fluorobenzyl)isoxazole- 3-carboxamide | LCMS (ESI) m/z (M + 1): 577.35 ¹H NMR (400 MHz, DMSO) δ 12.89 (s, 1H), 9.34 (t, J = 6.1 Hz, 1H), 8.46 (d, J = 4.7 Hz, 1H), 7.65-7.41 (m, 3H), 7.09 (d, J = 4.7 Hz, 1H), 6.57 (s, 1H), 6.00 (d, J = 8.2 Hz, 1H), 4.63-4.47 (m, 2H), 3.59 (s, 1H), 3.16 (dd, J = 12.0, 4.6 Hz, 1H), 2.97-2.82 (m, 2H), 2.78 (s, 6H), 1.51-1.39 (m, 2H), 1.41- 1.10 (m, 10H), 1.02 (d, J = 11.0 Hz, 1H), 0.65 (d, J = 6.7 Hz, 3H) |
| 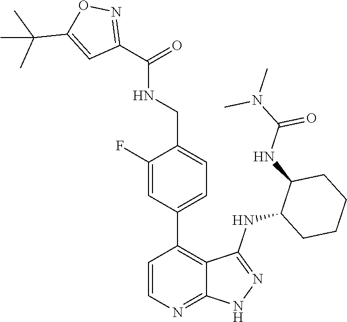 [SMILES: CN(C)C(=O)N[C@H]1CCCC[C@@H]1Nc1n[nH]c2nccc(-c3ccc(CNC(=O)c4cc(C(C)(C)C)on4)c(F)c3)c12] | 5-(tert-butyl)-N-(4-(3- (((1S,2S)-2-(3,3- dimethylureido)cyclo- hexyl)amino)-1H- pyrazolo[3,4-b]pyridin- 4-yl)-2-fluorobenzyl)- isoxazole-3-carboxamide | LCMS (ESI) m/z (M + 1): 577.40 ¹H NMR (400 MHz, DMSO) δ 12.33 (s, 1H), 9.21 (t, J = 5.5 Hz, 1H), 8.39 (d, J = 4.7 Hz, 1H), 7.51 (t, J = 7.9 Hz, 1H), 7.35 (d, J = 10.8 Hz, 1H), 7.30 (d, J = 7.6 Hz, 1H), 6.90 (d, J = 4.6 Hz, 1H), 6.60 (s, 1H), 6.04 (d, J = 7.6 Hz, 1H), 4.62 (dd, J = 14.9, 5.6 Hz, 1H), 4.51 (dd, J = 15.1, 5.2 Hz, 1H), 4.25 (d, J = 5.0 Hz, 1H), 3.45-3.36 (m, 2H), 2.57 (s, 6H), 2.34 (d, J = 9.7 Hz, 1H), 1.78 (d, J = 7.3 Hz, 1H), 1.63 (s, 2H), 1.43-0.98 (m. 13H) |
| 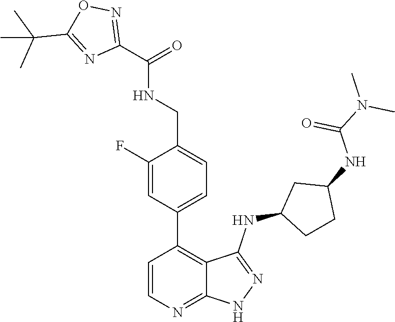 [SMILES: CN(C)C(=O)N[C@H]1CC[C@@H](Nc2n[nH]c3nccc(-c4ccc(CNC(=O)c5noc(C(C)(C)C)n5)c(F)c4)c23)C1] | 5-(tert-butyl)-N-(4-(3- (((1R,3S)-3-(3,3- dimethylureido)cyclo- pentyl)amino)-1H- pyrazolo[3,4-b]pyridin- 4-yl)-2-fluorobenzyl)- 1,2,4-oxadiazole-3- carboxamide | LCMS (ESI) m/z (M + 1): 564.36 ¹H NMR (400 MHz, DMSO) δ 12.45 (s, 1H), 9.56 (t, J = 6.0 Hz, 1H), 8.41 (d, J = 4.7 Hz, 1H), 7.58-7.42 (m, 3H), 6.96 (d, J = 4.7 Hz, 1H), 5.97 (d, J = 7.0 Hz, 1H), 4.59 (d, J = 6.0 Hz, 2H), 4.16 (d, J = 6.5 Hz, 1H), 3.90-3.77 (m, 2H), 2.72 (s, 6H), 2.30-2.22 (m, 1H), 1.93- 1.84 (m, 1H), 1.75 (dd, J = 12.4, 5.1 Hz, 1H), 1.54-1.14 (m, 12H) |
| 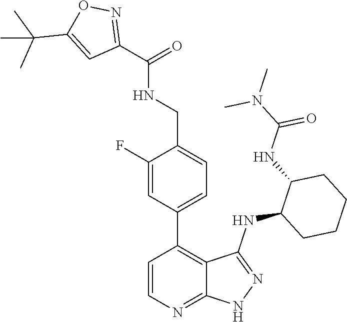 [SMILES: CN(C)C(=O)N[C@@H]1CCCC[C@H]1Nc1n[nH]c2nccc(-c3ccc(CNC(=O)c4cc(C(C)(C)C)on4)c(F)c3)c12] | 5-(tert-butyl)-N-(4-(3- (((1R,2R)-2-(3,3- dimethylureido)cyclo- hexyl)amino)-1H- pyrazolo[3,4-b]pyridin- 4-yl)-2-fluorobenzyl)- isoxazole-3-carboxamide | LCMS (ESI) m/z (M + 1): 577.39 ¹H NMR (400 MHz, DMSO) δ 12.45 (s, 1H), 9.21 (t, J = 5.6 Hz, 1H), 8.42 (d, J = 4.8 Hz, 1H), 7.52 (t, J = 7.8 Hz, 1H), 7.37 (d, J = 10.6 Hz, 1H), 7.31 (dd, J = 7.8, 1.4 Hz, 1H), 6.92 (d, J = 4.8 Hz, 1H), 6.60 (s, 1H), 6.05 (s, 1H), 4.62 (dd, J = 15.0, 5.9 Hz, 1H), 4.52 (dd, J = 15.0, 5.3 Hz, 1H), 3.40 (td, J = 10.5, 3.7 Hz, 1H), 3.31 (s, 1H), 2.57 (s, 6H), 2.32 (d, J = 12.0 Hz, 1H), 1.78 (d, J = 12.0 Hz, 1H), 1.63 (s, 2H), 1.37-0.95 (m, 13H) |
| 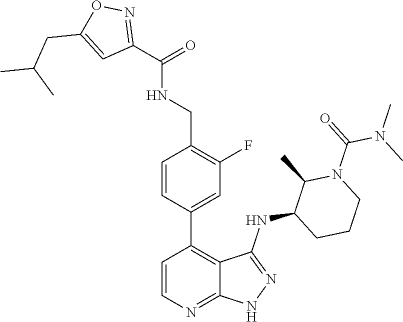 [SMILES: CC(C)Cc1cc(C(=O)NCc2ccc(-c3ccnc4[nH]nc(N[C@@H]5CCCN(C(=O)N(C)C)[C@@H]5C)c34)cc2F)no1] | N-(4-(3-(((2R,3R)-1- (dimethylcarbamoyl)-2- methylpiperidin-3-yl)- amino)-1H-pyrazolo- [3,4-b]pyridin-4-yl)-2- fluorobenzyl)-5- isobutylisoxazole-3- carboxamide | LCMS (ESI) m/z (M + 1): 577.57 ¹H NMR (400 MHz, DMSO) δ 12.47 (s, 1H), 9.35 (t, J = 6.0 Hz, 1H), 8.42 (d, J = 4.7 Hz, 1H), 7.55 (t, J = 7.8 Hz, 1H), 7.49-7.40 (m, 2H), 6.96 (d, J = 4.7 Hz, 1H), 6.60 (s, 1H), 4.56 (d, J = 6.0 Hz, 2H), 4.08 (dt, J = 13.3, 6.7 Hz, 1H), 3.82 (d, J = 7.3 Hz, 1H), 3.61 (dt, J = 11.5, 5.8 Hz, 1H), 3.17 (d, J = 12.9 Hz, 1H), 2.71 (d, J = 7.0 Hz, 2H), 2.68 (s, 6H), 2.01 (td, J = 13.5, 6.8 Hz, 1H), 1.69 (d, J = 9.3 Hz, 1H), 1.53 (d, J = 12.5 Hz, 1H), 1.45-1.36 (m, 1H), 1.29 (dt, J = 12.3, 8.3 Hz, 1H), 0.92 (d, J = 6.7 Hz, 6H), 0.83 (d, J = 6.8 Hz, 3H) |
| 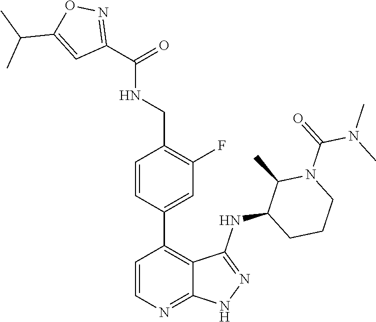 [SMILES: CC(C)c1cc(C(=O)NCc2ccc(-c3ccnc4[nH]nc(N[C@@H]5CCCN(C(=O)N(C)C)[C@@H]5C)c34)cc2F)no1] | N-(4-(3-(((2R,3R)-1- (dimethylcarbamoyl)-2- methylpiperidin-3- yl)amino)-1H-pyrazolo- [3,4-b]pyridin-4-yl)-2- fluorobenzyl)-5- isopropylisoxazole-3- carboxamide | LCMS (ESI) m/z (M + 1): 563.46 ¹H NMR (400 MHz, DMSO) δ 12.47 (s, 1H), 9.35 (t, J = 5.9 Hz, 1H), 8.43 (d, J = 4.7 Hz, 1H), 7.55 (t, J = 7.8 Hz, 1H), 7.44 (dd, J = 20.6, 9.3 Hz, 2H), 6.96 (d, J = 4.7 Hz, 1H), 6.59 (s, 1H), 4.56 (d, J = 5.8 Hz, 2H), 4.13-4.05 (m, 1H), 3.82 (d, J = 7.2 Hz, 1H), 3.61 (d, J = 4.6 Hz, 1H), 3.21-3.12 (m, 2H), 2.78-2.63 (m, 7H), 1.71-1.24 (m, 10H), 0.83 (d, J = 6.8 Hz, 3H) |
| 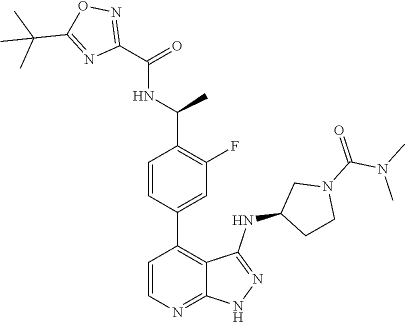 [SMILES: C[C@H](NC(=O)c1noc(C(C)(C)C)n1)c1ccc(-c2ccnc3[nH]nc(N[C@@H]4CCN(C(=O)N(C)C)C4)c23)cc1F] | 5-(tert-butyl)-N-((S)-1- (4-(3-(((R)-1-(dimethyl- carbamoyl)pyrrolidin-3- yl)amino)-1H-pyrazolo- [3,4-b]pyridin-4-yl)-2- fluorophenyl)ethyl)- 1,2,4-oxadiazole-3- carboxamide | LCMS (ESI) m/z (M + 1): 564.41 ¹H NMR (400 MHz, DMSO) δ 12.53 (s, 1H), 9.55 (d, J = 7.9 Hz, 1H), 8.43 (d, J = 4.7 Hz, 1H), 7.64 (t, J = 7.9 Hz, 1H), 7.53-7.41 (m, 2H), 7.00 (d, J = 4.7 Hz, 1H), 5.45 (p, J = 7.1 Hz, 1H), 4.32 (d, J = 5.7 Hz, 1H), 4.04 (dt, J = 10.0, 5.2 Hz, 1H), 3.50 (dd, J = 10.6, 5.6 Hz, 1H), 3.29-3.18 (m, 3H), 2.68 (s, 6H), 2.01 (td, J = 13.1, 7.5 Hz, 1H), 1.69 (td, J = 11.9, 5.8 Hz, 1H), 1.55 (d, J = 7.0 Hz, 3H), 1.43 (s, 9H) |
| 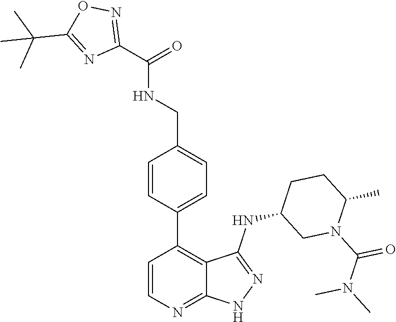 [SMILES: C[C@H]1CC[C@@H](Nc2n[nH]c3nccc(-c4ccc(CNC(=O)c5noc(C(C)(C)C)n5)cc4)c23)CN1C(=O)N(C)C] | 5-(tert-butyl)-N-(4-(3- (((3R,6S)-1-(dimethyl- carbamoyl)-6-methyl- piperidin-3-yl)amino)- 1H-pyrazolo[3,4-b]- pyridin-4-yl)benzyl)- 1,2,4-oxadiazole-3- carboxamide | LCMS (ESI) m/z (M + 1): 560.57 ¹H NMR (400 MHz, DMSO) δ 12.43 (s, 1H), 9.55 (t, J = 6.2 Hz, 1H), 8.41 (d, J = 4.7 Hz, 1H), 7.56 (dd, J = 24.2, 8.1 Hz, 4H), 6.92 (d, J = 4.7 Hz, 1H), 4.55 (d, J = 6.1 Hz, 2H), 3.66 (s, 1H), 3.51 (d, J = 11.6 Hz, 2H), 2.65 (s, 6H), 2.58 (dd, J = 12.5, 9.7 Hz, 2H), 1.75- 1.68 (m, 1H), 1.64-1.55 (m, 1H), 1.43 (s, 9H), 1.40-1.34 (m, 1H), 1.00 (d, J = 6.7 Hz, 3H) |
| 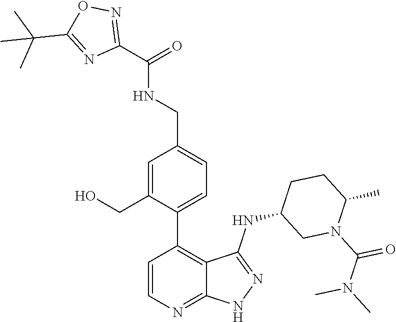 [SMILES: C[C@H]1CC[C@@H](Nc2n[nH]c3nccc(-c4ccc(CNC(=O)c5noc(C(C)(C)C)n5)cc4CO)c23)CN1C(=O)N(C)C] | 5-(tert-butyl)-N-(4-(3- (((3R,6S)-1-(dimethyl- carbamoyl)-6-methyl- piperidin-3-yl)amino)- 1H-pyrazolo[3,4-b]- pyridin-4-yl)-3-(hydroxy- methyl)benzyl)-1,2,4- oxadiazole-3-carboxamide | LCMS (ESI) m/z (M + 1): 590.72 ¹H NMR (400 MHz, DMSO) δ 12.37 (d, J = 2.5 Hz, 1H), 9.56 (t, J = 6.1 Hz, 1H), 8.39 (d, J = 4.7 Hz, 1H), 7.66 (s, 1H), 7.38 (d, J = 7.8 Hz, 2H), 7.22 (d, J = 7.7 Hz, 1H), 6.83 (d, J = 4.6 Hz, 1H), 5.25 (s, 1H), 4.54 (d, J = 6.1 Hz, 2H), 4.48 (s, 1H), 4.26 (s, 2H), 3.61 (s, 1H), 3.58-3.38 (m, 2H), 2.64 (s, 6H), 1.60-1.48 (m, 2H), 1.43 (s, 9H), 1.31-1.15 (m, 2H), 0.94 (d, J = 6.6 Hz, 3H) |
| 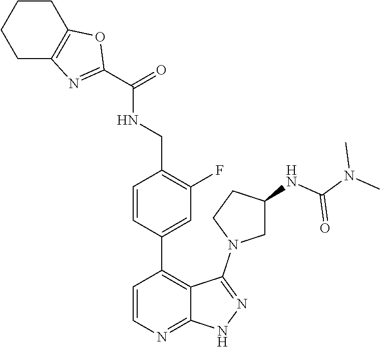 [SMILES: CN(C)C(=O)N[C@@H]1CCN(c2n[nH]c3nccc(-c4ccc(CNC(=O)c5nc6c(o5)CCCC6)c(F)c4)c23)C1] | (R)-N-(4-(3-(3-(3,3- dimethylureido)pyrrolidin- 1-yl)-1H-pyrazolo- [3,4-b]-pyridin-4-yl)- 2-fluorobenzyl)-4,5,6,7- tetrahydrobenzo[d]- oxazole-2-carboxamide | LCMS (ESI) m/z (M + 1): 547.53 ¹H NMR (400 MHz, DMSO) δ 12.79 (s, 1H), 9.41 (d, J = 5.8 Hz, 1H), 8.45 (d, J = 4.7 Hz, 1H), 7.59- 7.34 (m, 3H), 7.06 (d, J = 4.7 Hz, 1H), 6.04 (d, J = 6.8 Hz, 1H), 4.55 (d, J = 5.7 Hz, 2H), 4.01 (dd, J = 13.8, 6.7 Hz, 1H), 3.10 (dd, J = 9.7, 7.0 Hz, 1H), 2.87-2.78 (m, 2H), 2.74 (s, 6H), 2.68 (s, 4H), 1.88-1.74 (m, 5H), 1.59-1.48 (m, 1H) |
| 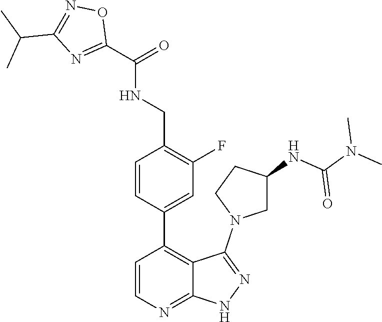 [SMILES: CC(C)c1noc(C(=O)NCc2ccc(-c3ccnc4[nH]nc(N5CC[C@@H](NC(=O)N(C)C)C5)c34)cc2F)n1] | (R)-N-(4-(3-(3-(3,3- dimethylureido)pyrrolidin- 1-yl)-1H-pyrazolo[3,4-b]- pyridin-4-yl)-2-fluoro- benzyl)-3-isopropyl-1,2,4- oxadiazole-5-carboxamide | LCMS (ESI) m/z (M + 1): 536.24 ¹H NMR (400 MHz, DMSO) δ 12.80 (s, 1H), 9.95 (t, J = 5.8 Hz, 1H), 8.46 (d, J = 4.7 Hz, 1H), 7.51 (ddt, J = 9.4, 7.9, 4.8 Hz, 3H), 7.07 (d, J = 4.7 Hz, 1H), 6.05 (d, J = 6.8 Hz, 1H), 4.60 (qd, J = 15.4, 5.9 Hz, 2H), 4.01 (dd, J = 14.4, 6.7 Hz, 1H), 3.16 (dt, J = 13.8, 6.9 Hz, 1H), 3.07 (dd, J = 9.8, 6.9 Hz, 1H), 2.84 (dd, J = 16.3, 8.2 Hz, 1H), 2.73 (s, 6H), 2.71-2.64 (m, 2H), 1.85 (dd, J = 12.5, 7.1 Hz, 1H), 1.60-1.51 (m, 1H), 1.31 (d, J = 6.9 Hz, 6H) |
| 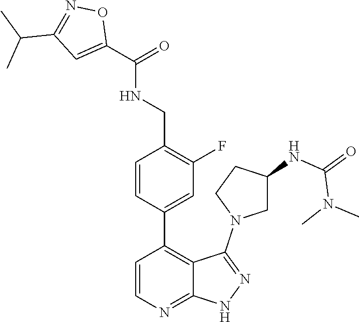 [SMILES: CC(C)c1cc(C(=O)NCc2ccc(-c3ccnc4[nH]nc(N5CC[C@@H](NC(=O)N(C)C)C5)c34)cc2F)on1] | (R)-N-(4-(3-(3-(3,3- dimethylureido)pyrrolidin- 1-yl)-1H-pyrazolo[3,4-b]- pyridin-4-yl)-2-fluoro- benzyl)-3-isopropyl- isoxazole-5-carboxamide | LCMS (ESI) m/z (M + 1): 535.23 ¹H NMR (400 MHz, DMSO) δ 12.80 (s, 1H), 9.49 (t, J = 5.9 Hz, 1H), 8.46 (d, J = 4.7 Hz, 1H), 7.62-7.39 (m, 3H), 7.10 (s, 1H), 7.06 (d, J = 4.7 Hz, 1H), 6.06 (d, J = 6.6 Hz, 1H), 4.65-4.51 (m, 2H), 4.06-3.96 (m, 1H), 3.11-3.02 (m, 2H), 2.87- 2.80 (m, 1H), 2.74 (s, 6H), 2.70- 2.63 (m, 1H), 1.85 (dt, J = 14.6, 7.6 Hz, 1H), 1.59-1.51 (m, 1H), 1.24 (d, J = 6.9 Hz, 6H) |
| 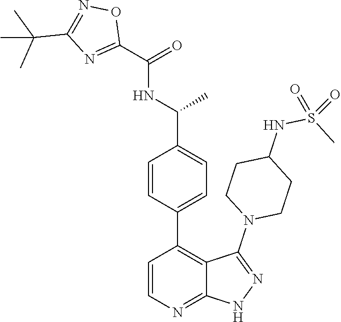 [SMILES: C[C@@H](NC(=O)c1nc(C(C)(C)C)no1)c1ccc(-c2ccnc3[nH]nc(N4CCC(NS(C)(=O)=O)CC4)c23)cc1] | (R)-3-(tert-butyl)-N- (1-(4-(3-(4-(methyl- sulfonamido)piperidin- 1-yl)-1H-pyrazolo- [3,4-b]pyridin-4-yl)- phenyl)ethyl)-1,2,4- oxadiazole-5-carboxamide | LCMS (ESI) m/z (M + 1): 567.25 ¹H NMR (400 MHz, DMSO) δ 12.85 (s, 1H), 9.87 (d, J = 8.0 Hz, 1H), 8.46 (d, J = 4.7 Hz, 1H), 7.62 (dd, J = 35.3, 8.2 Hz, 4H), 7.07 (d, J = 4.7 Hz, 1H), 6.96 (d, J = 6.9 Hz, 1H), 5.29-5.16 (m, 1H), 3.15-2.94 (m, 4H), 2.87 (s, 3H), 2.57 (dd, J = 13.9, 7.4 Hz, 2H), 1.59 (d, J = 7.0 Hz, 3H), 1.53 (s, 1H), 1.37 (s, 9H), 1.31 (dd, J = 20.7, 8.9 Hz, 2H) |
| 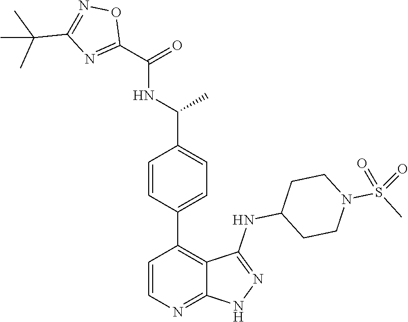 [SMILES: C[C@@H](NC(=O)c1nc(C(C)(C)C)no1)c1ccc(-c2ccnc3[nH]nc(NC4CCN(S(C)(=O)=O)CC4)c23)cc1] | (R)-3-(tert-butyl)-N- (1-(4-(3-((1-(methyl- sulfonyl)piperidin-4- yl)amino)-1H-pyrazolo- [3,4-b]pyridin-4-yl)- phenyl)ethyl)-1,2,4- oxadiazole-5-carboxamide | LCMS (ESI) m/z (M + 1): 567.34 ¹H NMR (400 MHz, DMSO) δ 12.47 (s, 1H), 9.91 (d, J = 8.0 Hz, 1H), 8.42 (d, J = 4.7 Hz, 1H), 7.61 (s, 4H), 6.95 (d, J = 4.7 Hz, 1H), 5.33- 5.13 (m, 1H), 4.00 (d, J = 6.8 Hz, 1H), 3.48-3.38 (m, 2H), 3.30-3.21 (m, 2H), 2.83 (s, 3H), 2.81-2.76 (m, 2H), 1.90 (s, 2H), 1.58 (d, J = 7.0 Hz, 3H), 1.41 (d, J = 3.8 Hz, 1H), 1.37 (s, 9H) |
| 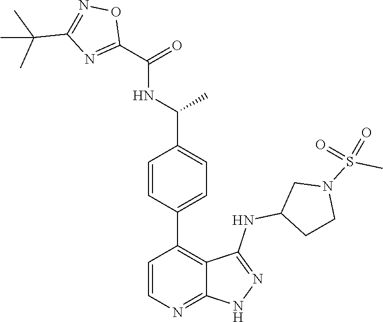 [SMILES: C[C@@H](NC(=O)c1nc(C(C)(C)C)no1)c1ccc(-c2ccnc3[nH]nc(NC4CCN(S(C)(=O)=O)C4)c23)cc1] | 3-(tert-butyl)-N-((1R)- 1-(4-(3-((1-(methyl- sulfonyl)pyrrolidin-3- yl)amino)-1H-pyrazolo- [3,4-b]pyridin-4-yl)- phenyl)ethyl)-1,2,4- oxadiazole-5-carboxamide | LCMS (ESI) m/z (M + 1): 553.37 ¹H NMR (400 MHz, DMSO) δ 12.55 (s, 1H), 9.89 (d, J = 8.1 Hz, 1H), 8.43 (d, J = 4.7 Hz, 1H), 7.60 (s, 4H), 6.96 (d, J = 4.7 Hz, 1H), 5.35- 5.18 (m, 1H), 4.37-4.25 (m, 1H), 4.12 (d, J = 4.4 Hz, 1H), 3.47 (dd, J = 10.5, 5.6 Hz, 1H), 3.23-3.11 (m, 3H), 2.76 (s, 3H), 2.11 (td, J = 13.6, 7.7 Hz, 1H), 1.77 (dd, J = 11.4, 6.1 Hz, 1H), 1.58 (d, J = 7.0 Hz, 3H), 1.37 (s, 9H) |
| 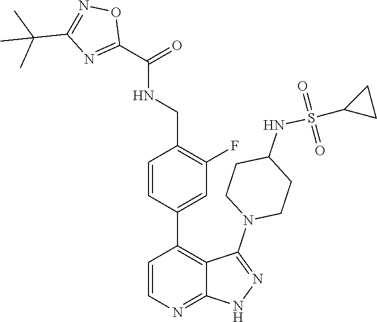 | 3-(tert-butyl)-N-(4-(3- (4-(cyclopropanesulfon- amido)piperidin-1-yl)-1H- pyrazolo[3,4-b]pyridin- 4-yl)-2-fluorobenzyl)- 1,2,4-oxadiazole-5- carboxamide | LCMS (ESI) m/z (M + 1): 493.59 ¹H NMR (400 MHz, DMSO) δ 12.93 (s, 1H), 9.93 (t, J = 5.9 Hz, 1H), 8.48 (d, J = 4.7 Hz, 1H), 7.67-7.44 (m, 3H), 7.11 (d, J = 4.7 Hz, 1H), 7.08 (d, J = 7.2 Hz, 1H), 4.61 (d, J = 5.9 Hz, 2H), 3.16 (s, 1H), 3.05 (d, J = 12.4 Hz, 2H), 2.69-2.57 (m, 2H), 2.57-2.52 (m, 2H), 1.65 (d, J = 9.8 Hz, 2H), 1.37 (s, 9H), 0.89 (dd, J = 6.1, 4.3 Hz, 4H) |
| 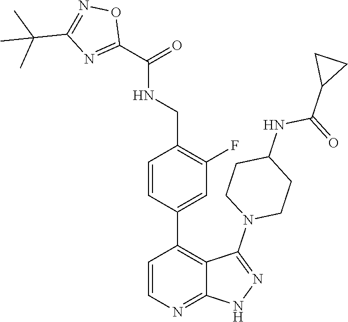 [SMILES: CC(C)(C)c1noc(C(=O)NCc2ccc(-c3ccnc4[nH]nc(N5CCC(NC(=O)C6CC6)CC5)c34)cc2F)n1] | 3-(tert-butyl)-N-(4-(3-(4- (cyclopropanecarbox- amido)piperidin-1-yl)- 1H-pyrazolo[3,4-b]- pyridin-4-yl)-2-fluoro- benzyl)-1,2,4-oxadiazole- 5-carboxamide | LCMS (ESI) m/z (M + 1): 561.28 ¹H NMR (400 MHz, DMSO) δ 12.94 (s, 1H), 9.97 (t, J = 5.6 Hz, 1H), 8.48 (d, J = 4.7 Hz, 1H), 7.91 (d, J = 7.5 Hz, 1H), 7.62-7.51 (m, 3H), 7.11 (d, J = 4.7 Hz, 1H), 4.62 (d, J = 5.6 Hz, 2H), 3.55 (s, 1H), 3.05 (d, J = 12.6 Hz, 2H), 2.60 (t, J = 10.6 Hz, 2H), 1.62-1.48 (m, 3H), 1.36 (s, 9H), 1.31-1.18 (m, 2H), 0.69-0.56 (m, 4H) |
| 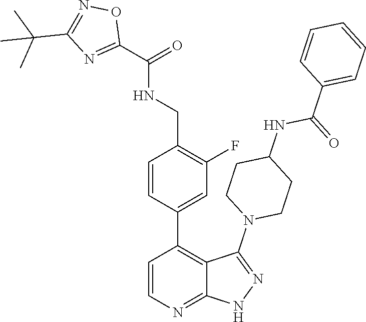 [SMILES: CC(C)(C)c1noc(C(=O)NCc2ccc(-c3ccnc4[nH]nc(N5CCC(NC(=O)c6ccccc6)CC5)c34)cc2F)n1] | N-(4-(3-(4-benzamido- piperidin-1-yl)-1H- pyrazolo[3,4-b]pyridin- 4-yl)-2-fluorobenzyl)-3- (tert-butyl)-1,2,4- oxadiazole-5-carboxamide | LCMS (ESI) m/z (M + 1): 597.29 ¹H NMR (400 MHz, DMSO) δ 12.94 (s, 1H), 9.96 (t, J = 6.0 Hz, 1H), 8.49 (d, J = 4.7 Hz, 1H), 8.19 (d, J = 7.4 Hz, 1H), 7.92-7.80 (m, 2H), 7.63-7.49 (m, 4H), 7.44 (t, J = 7.4 Hz, 1H), 7.12 (d, J = 4.7 Hz, 1H), 4.61 (d, J = 5.8 Hz, 2H), 3.76 (s, 1H), 3.12 (d, J = 12.2 Hz, 2H), 2.65 (t, J = 10.6 Hz, 2H), 1.62 (d, J = 9.4 Hz, 2H), 1.52-1.39 (m, 2H), 1.32 (s, 9H) |

### Example 38: (R)—N-(1-(4-(3-(1,1-dioxidothiomorpholino)-1H-pyrazolo[3,4-b]pyridin-4-yl)phenyl)ethyl)-3-isopropyl-1,2,4-oxadiazole-5-carboxamide

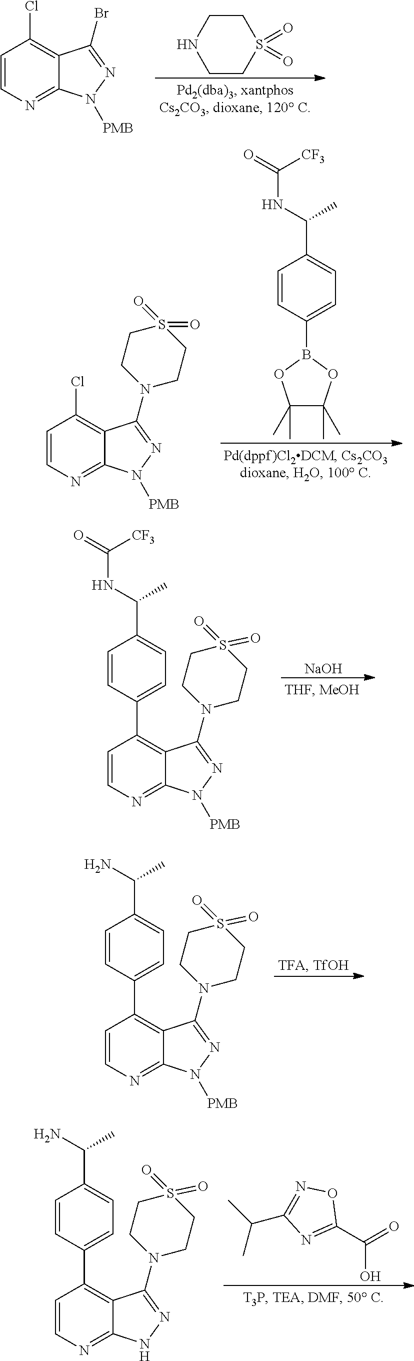

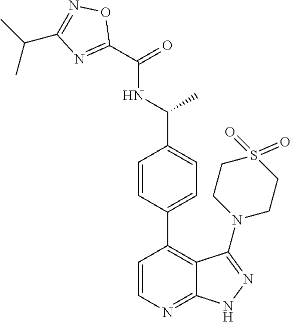 [SMILES: CC(C)c1noc(C(=O)N[C@H](C)c2ccc(-c3ccnc4[nH]nc(N5CCS(=O)(=O)CC5)c34)cc2)n1]

At 120° C. under N₂ atmosphere, to a stirred solution of 3-bromo-4-chloro-1-(4-methoxybenzyl)-1H-pyrazolo[3,4-b]pyridine (1.0 g, 2.84 mmol) in dioxane (20 mL) were added thiomorpholine 1,1-dioxide (460 mg, 3.4 mmol), Pd₂(dba)₃ (130 mg, 0.14 mmol), xantphos (250 mg, 0.43 mmol) and Cs₂CO₃ (1.85 g, 5.69 mmol). After being stirred at 120° C. for 8 hr, the reaction mixture was cooled down to room temperature and filtered through a pad of celite. The filtration was quenched with H₂O and extracted with ethyl acetate (3×). The combined organic layers were washed with brine and dried over Na₂SO₄. Solvents were removed and the residue was purified by flash chromatography (silica gel, 0˜50% ethyl acetate in petroleum ether) to provide 4-(4-chloro-1-(4-methoxybenzyl)-1H-pyrazolo[3,4-b]pyridin-3-yl)thiomorpholine 1,1-dioxide (574 mg, 57%) as a yellow solid. LCMS (ESI) m/z (M+1) 407.22.

At 100° C. under N₂ atmosphere, to a stirred solution of 4-(4-chloro-1-(4-methoxybenzyl)-1H-pyrazolo[3,4-b]pyridin-3-yl)thiomorpholine 1,1-dioxide (574 mg, 1.41 mmol) in dioxane/H₂O (20 mL/5 mL) were added 2,2,2-trifluoro-N-(2-fluoro-4-(4,4,5,5-tetramethyl-1,3,2-dioxaborolan-2-yl)benzyl)acetamide (727 mg, 2.1 mmol), Pd(dppf)Cl₂.DCM (115 mg, 0.14 mmol), and Cs₂CO₃(1.38 g, 4.2 mmol). After being stirred at 100° C. overnight, the reaction mixture was cooled down to room temperature and filtered through a pad of celite. The filtration was quenched with H₂O and extracted with ethyl acetate (3×). The combined organic layers were washed with brine and dried over Na₂SO₄. Solvents were removed and the residue was purified by flash chromatography (silica gel, 0˜50% ethyl acetate in petroleum ether) to provide (R)—N-(1-(4-(3-(1,1-dioxidothiomorpholino)-1-(4-methoxybenzyl)-1H-pyrazolo[3,4-b]pyridin-4-yl)phenyl)ethyl)-2,2,2-trifluoroacetamide which was dissolved in MeOH (15 mL) followed by adding NaOH (15 mL, 4N). After being stirred at room temperature overnight, the reaction mixture was extracted with ethyl acetate (3×). The organic layers were washed with brine and dried over Na₂SO₄. Solvents were removed under vacuum to afford (R)-4-(4-(4-(1-aminoethyl)phenyl)-1-(4-methoxybenzyl)-1H-pyrazolo[3,4-b]pyridin-3-yl)thiomorpholine 1,1-dioxide (461 mg, 80%) as a yellow solid. LCMS (ESI) m/z (M+1): 492.27.

At 0° C., to a stirred solution of (R)-4-(4-(4-(1-aminoethyl)phenyl)-1-(4-methoxybenzyl)-1H-pyrazolo[3,4-b]pyridin-3-yl)thiomorpholine 1,1-dioxide (120 mg, 0.24 mmol) in TFA (1 mL) was added TfOH (0.5 mL). After being stirred at room temperature for 4 hr, the reaction mixture was concentrated. The residue was quenched with sat. NaHCO₃ and extracted with DCM (3×). The combined organic layers were washed with brine and dried over Na₂SO₄. Solvents were removed under vacuum to provide (R)-4-(4-(4-(1-aminoethyl)phenyl)-1H-pyrazolo[3,4-b]pyridin-3-yl)thiomorpholine 1,1-dioxide (80 mg, 67%) as a white solid. LCMS (ESI) m/z (M+1): 372.27.

At 50° C., to a stirred solution of (R)-4-(4-(4-(1-aminoethyl)phenyl)-1H-pyrazolo[3,4-b]pyridin-3-yl)thiomorpholine 1,1-dioxide (80 mg, 0.22 mmol) in DMF (5 mL) were added 3-isopropyl-1,2,4-oxadiazole-5-carboxylic acid (41 mg, 0.26 mmol), T3P (0.2 mL, 0.66 mmol, 50% in ethyl acetate) and TEA (0.1 mL, 0.66 mmol). After being stirred at room temperature overnight, the reaction mixture was quenched with saturated NaHCO₃ and extracted with ethyl acetate (3×). The combined organic layers were washed with brine and dried over Na₂SO₄. Solvents were removed under vacuum and the crude product was purified by prep. HPLC (C18, 0˜90 acetonitrile in H₂O with 0.1% formic acid) to provide (R)—N-(1-(4-(3-(1,1-dioxidothiomorpholino)-1H-pyrazolo[3,4-b]pyridin-4-yl)phenyl)ethyl)-3-isopropyl-1,2,4-oxadiazole-5-carboxamide (13 mg, 16%) as a white solid. LCMS (ESI) m/z (M+1): 510.21. ¹H NMR (400 MHz, DMSO) δ 13.10 (s, 1H), 9.94 (d, J=7.9 Hz, 1H), 8.51 (d, J=4.7 Hz, 1H), 7.64 (dd, J=20.9, 8.3 Hz, 4H), 7.11 (d, J=4.7 Hz, 1H), 5.31-5.15 (m, 1H), 3.21 (d, J=11.8 Hz, 4H), 3.15 (dd, J=13.9, 6.9 Hz, 1H), 2.86 (s, 4H), 1.57 (d, J=7.1 Hz, 3H), 1.32 (d, J=6.9 Hz, 6H).

The following compounds were prepared by analogous methods:

| Chemical structure | Chemical names | LC-MS/HNMR |
|---|---|---|
| 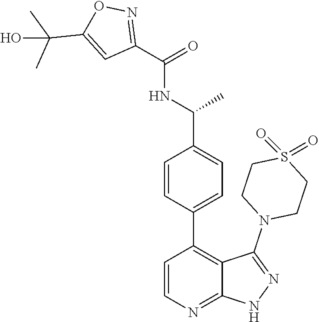 [SMILES: C[C@@H](NC(=O)c1cc(C(C)(C)O)on1)c1ccc(-c2ccnc3[nH]nc(N4CCS(=O)(=O)CC4)c23)cc1] | (R)-N-(1-(4-(3-(1,1- dioxidothiomorpholino)- 1H-pyrazolo[3,4-b]- pyridin-4-yl)phenyl)- ethyl)-5-(2-hydroxy- propan-2-yl)isoxazole- 3-carboxamide | LCMS (ESI) m/z (M + 1): 525.21 ¹H NMR (400 MHz, DMSO) δ 13.10 (s, 1H), 9.28 (d, J = 7.9 Hz, 2H), 8.50 (d, J = 4.7 Hz, 1H), 7.62 (dd, J = 28.7, 8.2 Hz, 4H), 7.11 (d, J = 4.7 Hz, 1H), 6.61 (s, 1H), 5.70 (s, 1H), 5.24-5.15 (m, 1H), 3.26-3.20 (m, 4H), 2.95-2.87 (m, 4H), 1.53 (d, J = 7.1 Hz, 3H), 1.49 (s, 6H) |
| 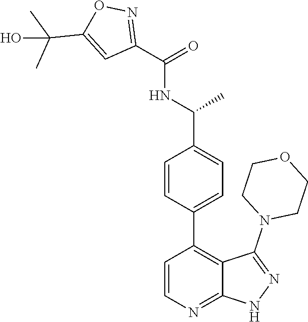 [SMILES: C[C@@H](NC(=O)c1cc(C(C)(C)O)on1)c1ccc(-c2ccnc3[nH]nc(N4CCOCC4)c23)cc1] | (R)-5-(2-hydroxypropan- 2-yl)-N-(1-(4-(3-morpholino- 1H-pyrazolo[3,4-b]pyridin- 4-yl)phenyl)ethyl)isoxazole- 3-carboxamide | LCMS (ESI) m/z (M + 1): 477.25 ¹H NMR (400 MHz, DMSO) δ 12.92 (s, 1H), 9.27 (d, J = 8.0 Hz, 1H), 8.47 (d, J = 4.7 Hz, 1H), 7.61 (dd, J = 32.0, 8.2 Hz, 4H), 7.09 (d, J = 4.7 Hz, 1H), 6.58 (s, 1H), 5.71 (s, 1H), 5.19 (dd, J = 14.6, 7.2 Hz, 1H), 3.41-3.34 (m, 4H), 2.71-2.63 (m, 4H), 1.52 (t, J = 6.7 Hz, 3H), 1.49 (s, 6H) |
| 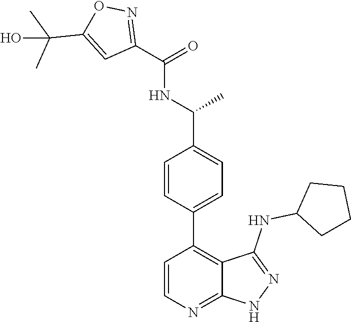 [SMILES: C[C@@H](NC(=O)c1cc(C(C)(C)O)on1)c1ccc(-c2ccnc3[nH]nc(NC4CCCC4)c23)cc1] | (R)-N-(1-(4-(3- (cyclopentylamino)-1H- pyrazolo[3,4-b]pyridin- 4-yl)phenyl)ethyl)-5-(2- hydroxypropan-2-yl)- isoxazole-3-carboxamide | LCMS (ESI) m/z (M + 1): 475.25 ¹H NMR (400 MHz, DMSO) δ 12.32 (s, 1H), 9.27 (d, J = 8.1 Hz, 1H), 8.39 (d, J = 4.7 Hz, 1H), 7.55 (dd, J = 23.6, 8.2 Hz, 4H), 6.91 (d, J = 4.7 Hz, 1H), 6.59 (s, 1H), 5.72 (s, 1H), 5.22 (p, J = 7.1 Hz, 1H), 3.87 (dt, J = 11.7, 5.9 Hz, 1H), 3.70 (d, J = 6.7 Hz, 1H), 1.86-1.71 (m, 2H), 1.53 (d, J = 7.1 Hz, 3H), 1.49 (s, 6H), 1.45 (dd, J = 11.7, 8.3 Hz, 4H), 1.27 (dt, J =11.5, 6.0 Hz, 2H) |
| 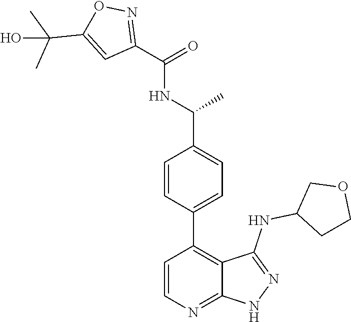 [SMILES: C[C@@H](NC(=O)c1cc(C(C)(C)O)on1)c1ccc(-c2ccnc3[nH]nc(NC4CCOC4)c23)cc1] | 5-(2-hydroxypropan-2- yl)-N-((1R)-1-(4-(3- ((tetrahydrofuran-3- yl)amino)-1H-pyrazolo- [3,4-b]pyridin-4-yl)- phenyl)ethyl)isoxazole- 3-carboxamide | LCMS (ESI) m/z (M + 1): 477.23 ¹H NMR (400 MHz, DMSO) δ 12.44 (s, 1H), 9.27 (d, J = 8.1 Hz, 1H), 8.41 (d, J = 4.7 Hz, 1H), 7.58 (t, J = 4.4 Hz, 4H), 6.95 (d, J = 4.7 Hz, 1H), 6.60 (s, 1H), 5.72 (s, 1H), 5.35-5.14 (m, 1H), 4.10 (ddd, J = 15.2, 8.9, 4.9 Hz, 2H), 3.83-3.74 (m, 1H), 3.68-3.55 (m, 2H), 3.44 (dd, J = 9.0, 3.3 Hz, 1H), 2.09 (tt, J = 8.7, 4.4 Hz, 1H), 1.61-1.56 (m, 1H), 1.54 (d, J = 7.0 Hz, 3H), 1.49 (s, 6H) |
| 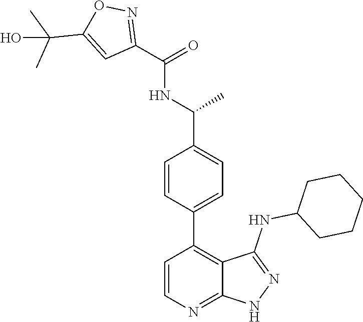 [SMILES: C[C@@H](NC(=O)c1cc(C(C)(C)O)on1)c1ccc(-c2ccnc3[nH]nc(NC4CCCCC4)c23)cc1] | (R)-N-(1-(4-(3-cyclo- hexylamino)-1H- pyrazolo[3,4-b]pyridin- 4-yl)phenyl)ethyl)-5- (2-hydroxypropan-2- yl)isoxazole-3-carboxamide | LCMS (ESI) m/z (M + 1): 489.32 ¹H NMR (400 MHz, DMSO) δ 12.30 (s, 1H), 9.27 (d, J = 8.1 Hz, 1H), 8.39 (d, J = 4.7 Hz, 1H), 7.56 (dd, J = 26.8, 8.0 Hz, 3H), 7.44-7.30 (m, 1H), 6.90 (d, J = 4.7 Hz, 1H), 6.59 (s, 1H), 5.72 (s, 1H), 5.27-5.18 (m, 1H), 3.69 (d, J = 7.5 Hz, 1H), 3.49- 3.36 (m, 1H), 1.86-1.69 (m, 2H), 1.53 (d, J = 7.0 Hz, 3H), 1.49 (s, 6H), 1.43-1.33 (m, 3H), 1.22-1.03 (m, 5H) |
| 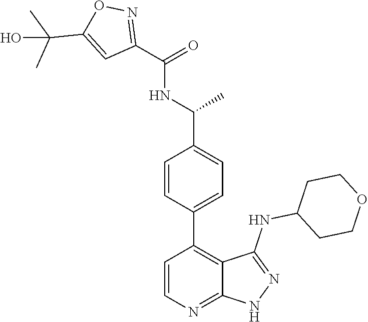 [SMILES: C[C@@H](NC(=O)c1cc(C(C)(C)O)on1)c1ccc(-c2ccnc3[nH]nc(NC4CCOCC4)c23)cc1] | (R)-5-(2-hydroxypropan- 2-yl)-N-(1-(4-(3-((tetra- hydro-2H-pyran-4-yl)- amino)-1H-pyrazolo- [3,4-b]pyridin-4-yl)- phenyl)ethyl)isoxazole- 3-carboxamide | LCMS (ESI) m/z (M + 1): 491.30 ¹H NMR (400 MHz, DMSO) δ 12.39 (s, 1H), 9.27 (d, J = 8.1 Hz, 1H), 8.40 (d, J = 4.7 Hz, 1H), 7.58 (q, J = 8.4 Hz, 4H), 6.93 (d, J = 4.7 Hz, 1H), 6.60 (s, 1H), 5.72 (s, 1H), 5.37- 5.13 (m, 1H), 3.86 (d, J = 7.3 Hz, 1H), 3.70-3.59 (m, 2H), 3.58-3.45 (m, 1H), 3.30-3.24 (m, 2H), 1.82 (d, J = 10.5 Hz, 2H), 1.53 (d, J = 7.1 Hz, 3H), 1.49 (s, 6H), 1.31- 1.17 (m, 2H). |
| 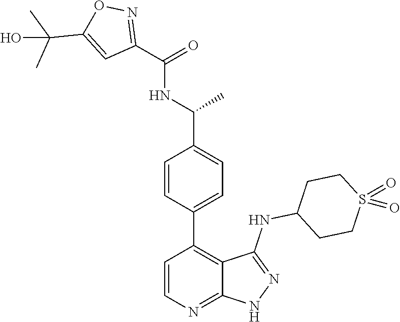 [SMILES: C[C@@H](NC(=O)c1cc(C(C)(C)O)on1)c1ccc(-c2ccnc3[nH]nc(NC4CCS(=O)(=O)CC4)c23)cc1] | (R)-N-(1-(4-(3-((1,1- dioxidotetrahydro-2H- thiopyran-4-yl)amino)- 1H-pyrazolo[3,4-b]- pyridin-4-yl)phenyl)- ethyl)-5-(2-hydroxy- propan-2-yl)isoxazole- 3-carboxamide | LCMS (ESI) m/z (M + l): 539.25 ¹H NMR (400 MHz, DMSO) δ 12.51 (s, 1H), 9.27 (d, J = 8.1 Hz, 1H), 8.42 (d, J = 4.7 Hz, 1H), 7.59 (dd, J = 19.2, 8.3 Hz, 3H), 6.96 (d, J = 4.7 Hz, 1H), 6.62 (s, 1H), 5.72 (s, 1H), 5.24 (dd, J = 14.8, 7.4 Hz, 1H), 4.24 (d, J = 6.2 Hz, 1H), 3.77- 3.56 (m, 1H), 3.15-3.02 (m, 2H), 3.01-2.93 (m, 2H), 2.20-2.10 (m, 2H), 2.00-1.87 (m, 2H), 1.54 (d, J = 7.1 Hz, 3H), 1.50 (s, 9H) |
| 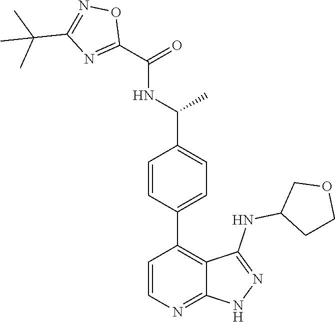 [SMILES: C[C@@H](NC(=O)c1nc(C(C)(C)C)no1)c1ccc(-c2ccnc3[nH]nc(NC4CCOC4)c23)cc1] | 3-(tert-butyl)-N-((1R)- 1-(4-(3-((tetrahydrofuran- 3-yl)amino)-1H-pyrazolo- [3,4-b]pyridin-4-yl)- phenyl)ethyl)-1,2,4- oxadiazole-5-carboxamide | LCMS (ESI) m/z (M + 1): 476.48 ¹H NMR (400 MHz, DMSO) δ 12.45 (s, 1H), 9.88 (d, J = 7.5 Hz, 1H), 8.41 (d, J = 4.7 Hz, 1H), 7.76- 7.47 (m, 4H), 6.95 (d, J = 4.7 Hz, 1H), 5.36-5.12 (m, 1H), 4.08 (dd, J = 11.2, 7.2 Hz, 2H), 3.77 (ddd, J = 8.9, 5.3, 1.4 Hz, 1H), 3.66- 3.57 (m, 2H), 3.43 (dd, J = 8.9, 3.2 Hz, 1H), 2.16-2.04 (m, 1H), 1.58 (d, J = 7.1 Hz, 3H), 1.55- 1.51 (m, 1H), 1.37 (s, 9H) |

### Example 39: (R)—N-(1-(4-(3-(4-acrylamidopiperidin-1-yl)-1H-pyrazolo[3,4-b]pyridin-4-yl)phenyl)ethyl)-5-(2-hydroxypropan-2-yl)isoxazole-3-carboxamide

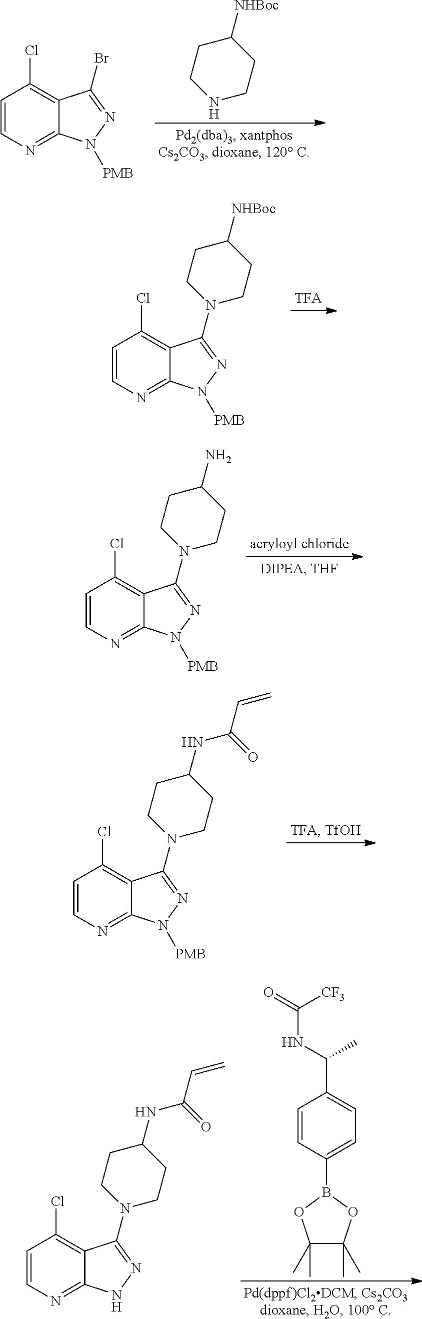

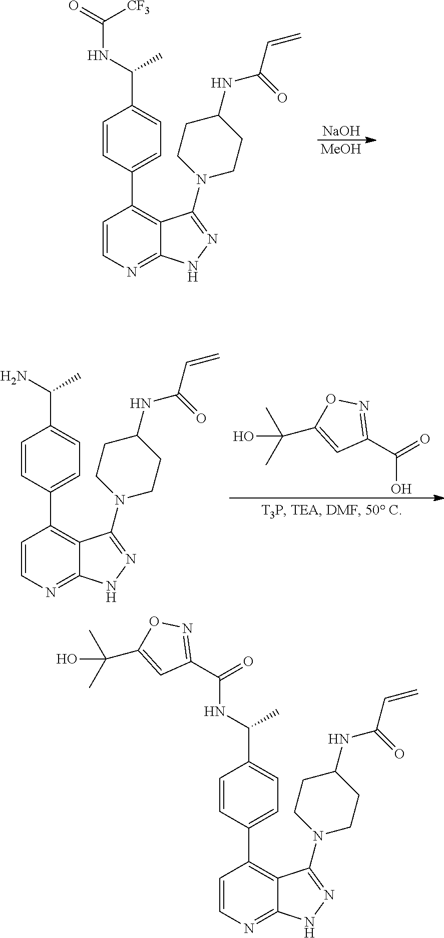

(R)—N-(1-(4-(3-(4-acrylamidopiperidin-1-yl)-1H-pyrazolo[3,4-b]pyridin-4-yl)phenyl)ethyl)-5-(2-hydroxypropan-2-yl)isoxazole-3-carboxamide (11 mg, 6%) was obtained as a white solid following a similar procedure to that outlined in Example 38. LCMS (ESI) m/z (M+1): 544.34. ¹H NMR (400 MHz, DMSO) δ 12.84 (s, 1H), 9.27 (d, J=8.1 Hz, 1H), 8.45 (d, J=4.7 Hz, 1H), 7.88 (d, J=7.6 Hz, 1H), 7.64 (d, J=8.2 Hz, 1H), 7.55 (d, J=8.2 Hz, 1H), 7.06 (d, J=4.7 Hz, 1H), 6.60 (s, 1H), 6.14 (ddd, J=19.3, 17.0, 6.1 Hz, 2H), 5.71 (s, 1H), 5.60-5.51 (m, 1H), 5.27-5.10 (m, 1H), 3.68-3.51 (m, 2H), 3.10-2.92 (m, 2H), 2.67-2.54 (m, 3H), 1.53 (d, J=7.0 Hz, 3H), 1.48 (s, 6H), 1.26-1.16 (m, 2H)

The following compounds were prepared by analogous methods:

| Chemical structure | Chemical names | LC-MS/HNMR |
|---|---|---|
| 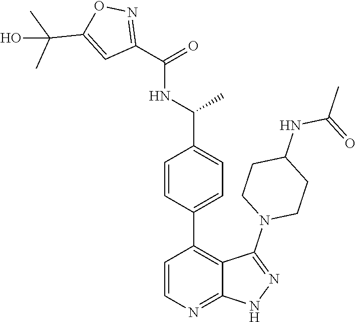 [SMILES: CC(=O)NC1CCN(c2n[nH]c3nccc(-c4ccc([C@@H](C)NC(=O)c5cc(C(C)(C)O)on5)cc4)c23)CC1] | (R)-N-(1-(4-(3-(4- acetamidopiperidin- 1-yl)-1H-pyrazolo- [3,4-b]pyridin-4- yl)phenyl)ethyl)-5- (2-hydroxypropan- 2-yl)isoxazole-3- carboxamide | LCMS (ESI) m/z (M + 1): 532.29 ¹H NMR (400 MHz, DMSO) δ 12.83 (s, 1H), 9.25 (d, J = 8.1 Hz, 1H), 8.45 (d, J = 4.7 Hz, 1H), 7.65-7.58 (m, 2H), 7.54 (d, J = 8.1 Hz, 2H), 7.06 (d, J = 4.7 Hz, 1H), 6.61 (s, 1H), 5.72 (s, 1H), 5.25-5.15 (m, 1H), 3.54-3.44 (m, 2H), 3.09-2.92 (m, 3H), 2.58-2.51 (m, 2H), 1.77 (s, 3H), 1.54 (d, J = 7.0 Hz, 3H), 1.49 (s, 6H), 1.26-1.12 (m, 2H) |
| 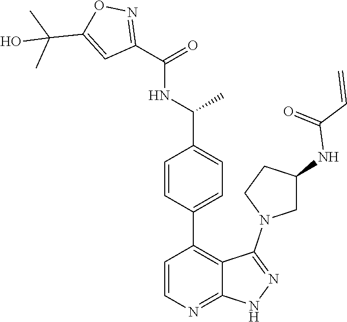 [SMILES: C=CC(=O)N[C@@H]1CCN(c2n[nH]c3nccc(-c4ccc([C@@H](C)NC(=O)c5cc(C(C)(C)O)on5)cc4)c23)C1] | N-((R)-1-(4-(3-((R)- 3-acrylamidopyrrolidin- 1-yl)-1H-pyrazolo- [3,4-b]pyridin-4-yl)- phenyl)ethyl)-5-(2- hydroxypropan-2-yl)- isoxazole-3-carboxamide | LCMS (ESI) m/z (M + 1): 530.26 ¹H NMR (400 MHz, DMSO) δ 12.77 (s, 1H), 9.22 (d, J = 8.2 Hz, 1H), 8.44 (d, J = 4.7 Hz, 2H), 8.14 (d, J = 7.0 Hz, 1H), 7.56 (dd, J = 36.4, 8.2 Hz, 3H), 7.04 (d, J = 4.7 Hz, 1H), 6.61 (s, 1H), 6.12 (ddd, J = 19.4, 17.1, 6.2 Hz, 2H), 5.72 (s, 1H), 5.55 (dd, J = 10.1, 2.3 Hz, 1H), 5.27-5.10 (m, 1H), 4.16 (d, J = 6.1 Hz, 1H), 3.15 (dd, J = 10.1, 7.0 Hz, 1H), 2.77 (dd, J = 10.3, 5.7 Hz, 1H), 2.69 (dd, J = 16.7, 7.2 Hz, 1H), 2.57 (dd, J = 14.7, 9.1 Hz, 1H), 2.06-1.94 (m, 1H), 1.86 (dd, J = 12.4, 7.3 Hz, 1H), 1.51 (d, J = 7.1 Hz, 3H), 1.49 (s, 6H), 1.46-1.41 (m, 1H) |
| 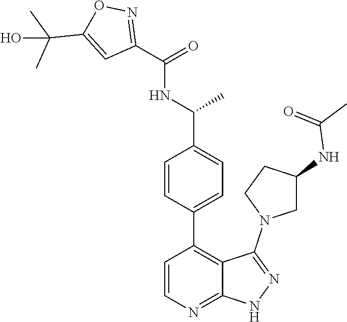 [SMILES: CC(=O)N[C@@H]1CCN(c2n[nH]c3nccc(-c4ccc([C@@H](C)NC(=O)c5cc(C(C)(C)O)on5)cc4)c23)C1] | N-((R)-1-(4-(3-((R)-3- acetamidopyrrolidin-1- yl)-1H-pyrazolo[3,4-b]- pyridin-4-yl)phenyl)- ethyl)-5-(2-hydroxy- propan-2-yl)isoxazole- 3-carboxamide | LCMS (ESI) m/z (M + 1): 518.28 ¹H NMR (400 MHz, DMSO) δ 12.75 (s, 1H), 9.24 (d, J = 8.1 Hz, 1H), 8.44 (d, J = 4.7 Hz, 1H), 7.87 (d, J = 7.0 Hz, 1H), 7.60 (d, J = 8.3 Hz, 2H), 7.52 (d, J = 8.2 Hz, 2H), 7.03 (d, J = 4.7 Hz, 1H), 6.60 (s, 1H), 5.73 (s, 1H), 5.31-5.13 (m, 1H), 4.06 (dd, J = 13.0, 6.5 Hz, 1H), 3.08 (dd, J = 10.1, 7.0 Hz, 1H), 2.75-2.63 (m, 2H), 2.63-2.54 (m, 1H), 1.84 (dd, J= 13.8, 6.2 Hz, 1H), 1.75 (s, 3H), 1.53 (d, J = 7.0 Hz, 3H), 1.49 (s, 6H), 1.44-1.33 (m, 2H) |
| 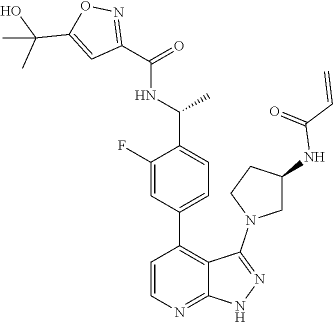 [SMILES: C=CC(=O)N[C@@H]1CCN(c2n[nH]c3nccc(-c4ccc([C@@H](C)NC(=O)c5cc(C(C)(C)O)on5)c(F)c4)c23)C1] | N-((R)-1-(4-(3-((R)-3- acrylamidopyrrolidin-1- yl)-1H-pyrazolo[3,4-b]- pyridin-4-yl)-2-fluoro- phenyl)ethyl)-5-(2- hydroxypropan-2-yl)- isoxazole-3-carboxamide | LCMS (ESI) m/z (M + 1): 548.31 ¹H NMR (400 MHz, DMSO) δ 12.83 (s, 1H), 9.31 (t, J = 7.0 Hz, 1H), 8.46 (d, J = 4.7 Hz, 1H), 8.15 (d, J = 6.9 Hz, 1H), 7.60 (t, J = 8.0 Hz, 1H), 7.53-7.41 (m, 2H), 7.20-6.94 (m, 1H), 6.61 (d, J = 2.0 Hz, 1H), 6.26-6.14 (m, 1H), 6.08-5.97 (m, 1H), 5.72 (s, 1H), 5.64-5.48 (m, 1H), 5.41 (t, J = 7.3 Hz, 1H), 4.26-4.12 (m, 1H), 3.20-3.12 (m, 2H), 2.84-2.68 (m, 2H), 2.68- 2.56 (m, 1H), 2.02-1.84 (m, 1H), 1.51 (d, J = 7.1 Hz, 3H), 1.49 (s, 6H) |
| 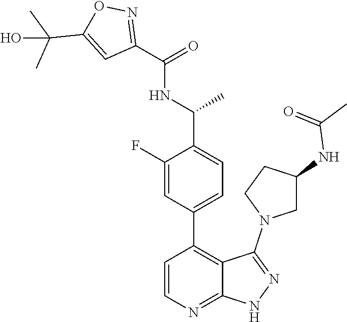 [SMILES: CC(=O)N[C@@H]1CCN(c2n[nH]c3nccc(-c4ccc([C@@H](C)NC(=O)c5cc(C(C)(C)O)on5)c(F)c4)c23)C1] | N-((R)-1-(4-(3-((R)-3- acetamidopyrrolidin- 1-yl)-1H-pyrazolo[3,4-b]- pyridin-4-yl)-2-fluoro- phenyl)ethyl)-5-(2- hydroxypropan-2-yl)- isoxazole-3-carboxamide | LCMS (ESI) m/z (M + 1): 536.33 ¹H NMR (400 MHz, DMSO) δ 12.83 (s, 1H), 9.33 (d, J = 7.8 Hz, 1H), 8.46 (d, J = 4.7 Hz, 1H), 7.89 (d, J = 6.9 Hz, 1H), 7.89 (d, J = 6.9 Hz, 1H), 7.60 (t, J = 7.9 Hz, 1H), 7.52- 7.39 (m, 2H), 7.08 (d, J = 4.7 Hz, 1H), 6.60 (d, J = 1.9 Hz, 1H), 5.73 (s, 1H), 5.58-5.29 (m, 1H), 4.07 (dd, J = 13.7, 6.7 Hz, 1H), 3.18- 3.03 (m, 1H), 2.78-2.67 (m, 2H), 2.67-2.57 (m, 1H), 2.50 (s, 3H), 1.92-1.81 (m, 1H), 1.75 (s, J = 9.0 Hz, 3H), 1.53 (d, J = 7.0 Hz, 1H), 1.49 (s, 6H) |
| 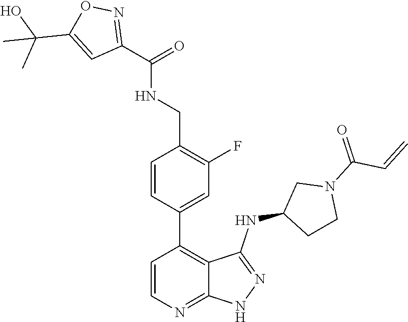 [SMILES: C=CC(=O)N1CC[C@@H](Nc2n[nH]c3nccc(-c4ccc(CNC(=O)c5cc(C(C)(C)O)on5)c(F)c4)c23)C1] | (R)-N-(4-(3-((1- acryloylpyrrolidin-3- yl)amino)-1H-pyrazolo- [3,4-b]pyridin-4-yl)-2- fluorobenzyl)-5-(2- hydroxypropan-2-yl)- isoxazole-3-carboxamide | LCMS (ESI) m/z (M + 1): 534.36 ¹H NMR (400 MHz, DMSO) δ 12.58 (s, 1H), 9.35 (d, J = 5.2 Hz, 1H), 8.43 (d, J = 4.7 Hz, 1H), 7.51-7.41 (m, 3H), 7.00 (dd, J = 4.7, 1.2 Hz, 1H), 6.64 (d, J = 3.6 Hz, 1H), 6.59- 6.44 (m, 1H), 6.10 (ddd, J = 16.8, 5.7, 2.4 Hz, 1H), 5.73 (d, J = 3.6 Hz, 1H), 5.62 (ddd, J = 10.3, 7.8, 2.4 Hz, 1H), 4.58-4.48 (m, 3H), 4.23-4.08 (m, 1H), 3.89-3.56 (m, 2H), 3.46-3.36 (m, 2H), 2.21-2.05 (m, 1H), 1.93-1.71 (m, 1H), 1.50 (s, 6H) |
| 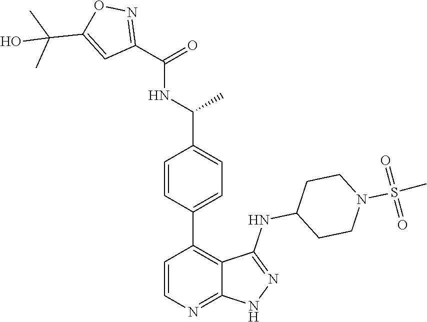 [SMILES: C[C@@H](NC(=O)c1cc(C(C)(C)O)on1)c1ccc(-c2ccnc3[nH]nc(NC4CCN(S(C)(=O)=O)CC4)c23)cc1] | (R)-5-(2-hydroxypropan- 2-yl)-N-(1-(4-(3-((1- (methylsulfonyl)piperidin- 4-yl)amino)-1H-pyrazolo- [3,4-b]pyridin-4-yl)- phenyl)ethyl)isoxazole- 3-carboxamide | LCMS (ESI) m/z (M + 1): 568.42 ¹H NMR (400 MHz, DMSO) δ 12.45 (s, 1H), 9.27 (d, J = 8.1 Hz, 1H), 8.41 (d, J = 4.7 Hz, 1H), 7.58 (s, 4H), 6.94 (d, J = 4.7 Hz, 1H), 6.61 (d, J = 5.3 Hz, 1H), 5.71 (br, 1H), 5.35-5.08 (m, 1H), 3.99 (d, J = 6.8 Hz, 1H), 3.25 (dd, J = 11.4, 4.8 Hz, 3H), 2.83 (s, 3H), 2.82-2.77 (m, 2H), 1.98-1.86 (m, 2H), 1.54 (d, J = 7.1 Hz, 3H), 1.49 (s, 6H), 1.41 (m, 2H) |
| 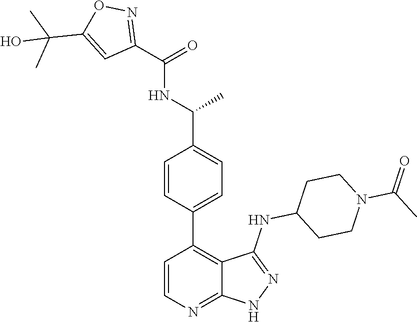 [SMILES: CC(=O)N1CCC(Nc2n[nH]c3nccc(-c4ccc([C@@H](C)NC(=O)c5cc(C(C)(C)O)on5)cc4)c23)CC1] | (R)-N-(1-(4-(3-((1- acetylpiperidin-4- yl)amino)-1H-pyrazolo- [3,4-b]pyridin-4-yl)- phenyl)ethyl)-5-(2- hydroxypropan-2-yl)- isoxazole-3-carboxamide | LCMS (ESI) m/z (M + 1): 532.26 ¹H NMR (400 MHz, DMSO) δ 12.42 (s, 1H), 9.26 (d, J = 8.1 Hz, 1H), 8.41 (d, J = 4.7 Hz, 1H), 7.63-7.52 (m, 4H), 6.93 (d, J = 4.7 Hz, 1H), 6.60 (s, 1H), 5.72 (s, 1H), 5.31- 5.15 (m, 1H), 3.91 (d, J = 6.9 Hz, 1H), 3.84 (d, J = 13.4 Hz, 1H), 3.58-3.46 (m, 2H), 3.13-3.01 (m, 1H), 2.84 (dd, J = 13.3, 9.8 Hz, 1H), 1.94 (s, 3H), 1.88-1.75 (m, 2H), 1.53 (d, J = 7.0 Hz, 3H), 1.49 (s, 6H), 1.32-1.09 (m, 2H) |

### Example 40: (R)—N-(4-(3-(3-(3,3-dimethylureido)pyrrolidin-1-yl)-1H-pyrazolo[3,4-b]pyridin-4-yl)-2-fluorobenzyl)-5-(2-hydroxypropan-2-yl)isoxazole-3-carboxamide

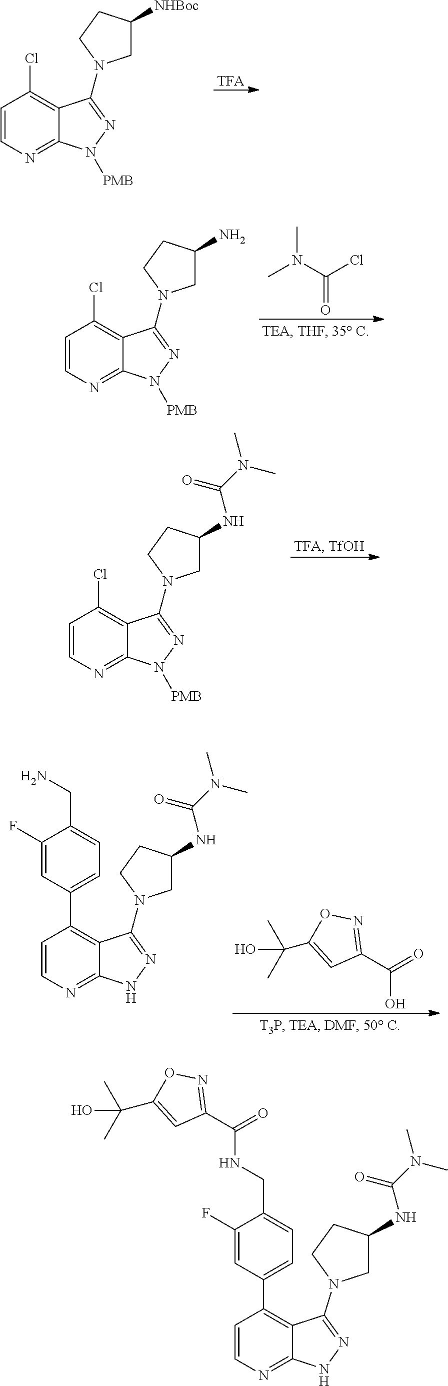

(R)—N-(4-(3-(3-(3,3-dimethylureido)pyrrolidin-1-yl)-1H-pyrazolo[3,4-b]pyridin-4-yl)-2-fluorobenzyl)-5-(2-hydroxypropan-2-yl)isoxazole-3-carboxamide (57 mg, 27%) was obtained as a yellow solid following an analogous procedure to that of Example 38. LCMS (ESI) m/z (M+1): 551.30. ¹H NMR (400 MHz, DMSO) δ 12.80 (s, 1H), 9.35 (t, J=5.9 Hz, 1H), 8.46 (d, J=4.7 Hz, 1H), 7.57-7.40 (m, 3H), 7.07 (d, J=4.7 Hz, 1H), 6.63 (s, 1H), 6.05 (d, J=6.8 Hz, 1H), 5.73 (s, 1H), 4.65-4.50 (m, 2H), 4.07-3.96 (m, 1H), 3.09 (dd, J=9.8, 7.0 Hz, 1H), 2.88-2.58 (m, 9H), 1.85 (td, J=14.8, 7.7 Hz, 1H), 1.63-1.36 (m, 7H)

The following compounds were prepared by analogous methods:

| Chemical structure | Chemical names | LC-MS/HNMR |
|---|---|---|
| 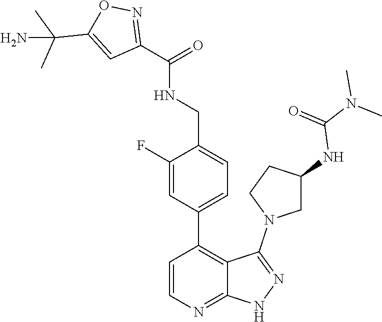 [SMILES: CN(C)C(=O)N[C@@H]1CCN(c2n[nH]c3nccc(-c4ccc(CNC(=O)c5cc(C(C)(C)N)on5)c(F)c4)c23)C1] | (R)-5-(2-aminopropan-2- yl)-N-(4-(3-(3-(3,3- dimethylureido)pyrrolidin- 1-yl)-1H-pyrazolo[3,4-b]- pyridin-4-yl)-2-fluoro- benzyl)isoxazole-3- carboxamide | LCMS (ESI) m/z (M + 1): 550.29 ¹H NMR (400 MHz, DMSO) δ 12.80 (s, 1H), 9.33 (t, J = 5.9 Hz, 1H), 8.45 (d, J = 4.7 Hz, 1H), 7.51-7.46 (m, 3H), 7.06 (d, J = 4.7 Hz, 1H), 6.65 (s, 1H), 6.05 (d, J = 6.8 Hz, 1H), 4.61-4.52 (m, 2H), 4.04-3.99 (m, 1H), 3.09 (dd, J = 9.9, 6.9 Hz, 1H), 2.84-2.73 (m, 8H), 2.67-2.62 (m, 1H), 1.87-1.81 (m, 1H), 1.57- 1.50 (m, 1H), 1.41 (s, 9H) |
| 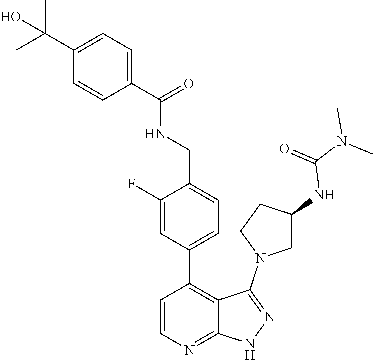 [SMILES: CN(C)C(=O)N[C@@H]1CCN(c2n[nH]c3nccc(-c4ccc(CNC(=O)c5ccc(C(C)(C)O)cc5)c(F)c4)c23)C1] | (R)-N-(4-(3-(3-(3,3- dimethylureido)pyrrolidin- 1-yl)-1H-pyrazolo[3,4-b]- pyridin-4-yl)-2-fluoro- benzyl)-4-(2-hydroxy- propan-2-yl)benzamide | LCMS (ESI) m/z (M + 1): 560.32 ¹H NMR (400 MHz, DMSO) δ 12.80 (s, 1H), 9.03 (t, J = 5.8 Hz, 1H), 8.45 (d, J = 4.7 Hz, 1H), 7.85 (d, J = 8.5 Hz, 2H), 7.56 (d, J = 8.5 Hz, 2H), 7.54-7.41 (m, 3H), 7.06 (d, J = 4.7 Hz, 1H), 6.07 (d, J = 6.8 Hz, 1H), 5.12 (s, 1H), 4.60 (qd, J = 15.5, 5.7 Hz, 2H), 4.10- 3.97 (m, 1H), 3.09 (dd, J = 9.8, 6.9 Hz, 1H), 2.97-2.54 (m, 9H), 1.87 (td, J = 14.7, 7.7 Hz, 1H), 1.61-1.50 (m, 1H), 1.44 (s, 6H) |
| 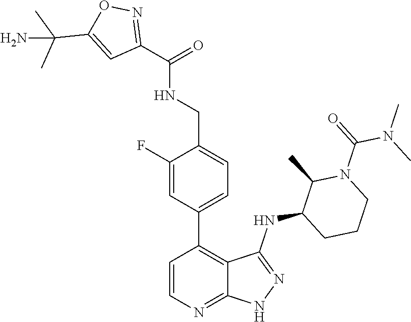 [SMILES: C[C@@H]1[C@H](Nc2n[nH]c3nccc(-c4ccc(CNC(=O)c5cc(C(C)(C)N)on5)c(F)c4)c23)CCCN1C(=O)N(C)C] | 5-(2-aminopropan-2-yl)- N-(4-(3-(((2R,3R)-1- (dimethylcarbamoyl)-2- methylpiperidin-3-yl)- amino)-1H-pyrazolo- [3,4-b]pyridin-4-yl)-2- fluorobenzyl)isoxazole- 3-carboxamide | LCMS (ESI) m/z (M + 1): 578.33 ¹H NMR (400 MHz, DMSO) δ 12.47 (s, 1H), 9.42 (t, J = 6.1 Hz, 1H), 8.43 (d, J = 4.7 Hz, 1H), 7.54 (t, J = 7.8 Hz, 1H), 7.47 (dd, J = 10.7, 1.4 Hz, 1H), 7.42 (dd, J = 7.8, 1.5 Hz, 1H), 6.96 (d, J = 4.7 Hz, 1H), 6.79 (s, 1H), 4.57 (d, J = 5.9 Hz, 2H), 4.11-4.05 (m, 1H), 3.83 (d, J = 7.2 Hz, 1H), 3.61 (dd, J = 11.5, 7.1 Hz, 1H), 3.19-3.15 (m, 1H), 2.80-2.63 (m, 7H), 1.69 (d, J = 9.3 Hz, 1H), 1.57-1.30 (m, 9H), 0.84 (d, J = 6.8 Hz, 3H) |
| 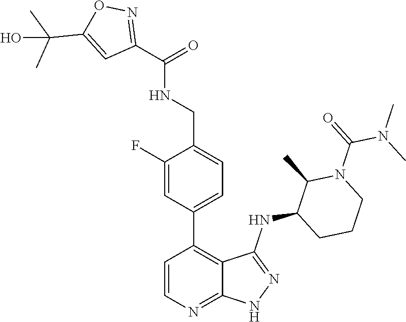 [SMILES: C[C@@H]1[C@H](Nc2n[nH]c3nccc(-c4ccc(CNC(=O)c5cc(C(C)(C)O)on5)c(F)c4)c23)CCCN1C(=O)N(C)C] | N-(4-(3-(((2R,3R)-1- (dimethylcarbamoyl)- 2-methylpiperidin-3- yl)amino)-1H-pyrazolo- [3,4-b]pyridin-4-yl)- 2-fluorobenzyl)-5-(2- hydroxypropan-2-yl)- isoxazole-3-carboxamide | LCMS (ESI) m/z (M + 1): 579.35 ¹H NMR (400 MHz, DMSO) δ 12.47 (s, 1H), 9.38 (t, J = 6.0 Hz, 1H), 8.43 (d, J = 4.7 Hz, 1H), 7.55 (t, J = 7.8 Hz, 1H), 7.47 (dd, J = 10.7, 1.4 Hz, 1H), 7.42 (dd, J = 7.8, 1.5 Hz, 1H), 6.96 (d, J = 4.7 Hz, 1H), 6.62 (s, 1H), 5.73 (s, 1H), 4.56 (d, J = 6.0 Hz, 2H), 4.08 (dd, J = 12.4, 6.3 Hz, 1H), 3.82 (d, J = 7.3 Hz, 1H), 3.61 (dt, J = 11.3, 4.3 Hz, 1H), 3.17 (d, J = 13.0 Hz, 1H), 2.84-2.56 (m, 7H), 1.69 (d, J = 8.8 Hz, 1H), 1.56-1.23 (m, 9H), 0.84 (d, J = 6.8 Hz, 3H) |

### Example 42: 5-(tert-butyl)-N-(4-(3-(2,5-dihydro-1H-pyrrol-3-yl)-1H-pyrazolo[3,4-b]pyridin-4-yl)-2-fluorobenzyl)-1,2,4-oxadiazole-3-carboxamide

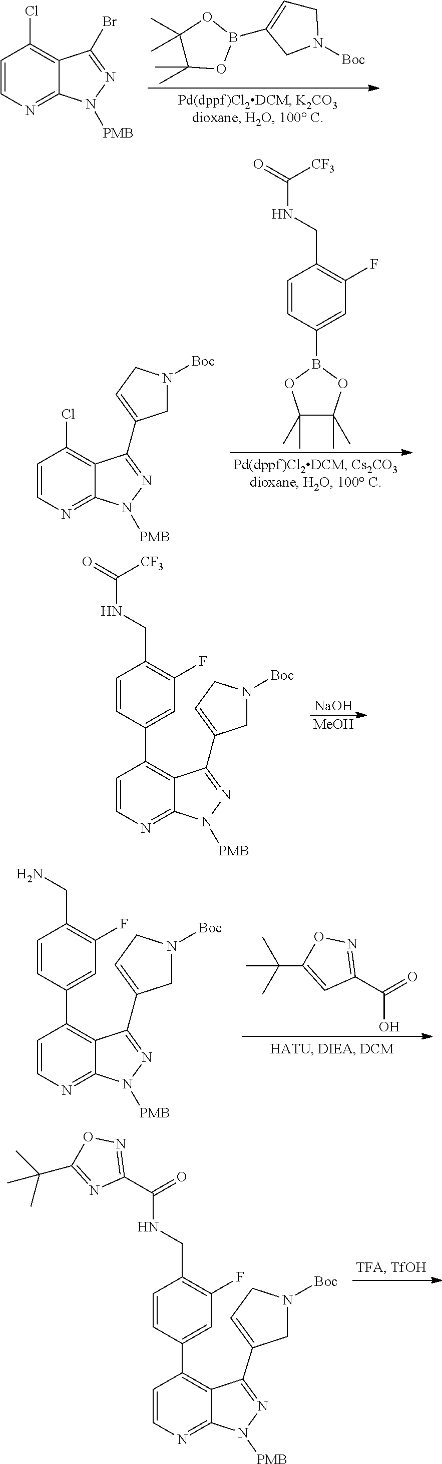

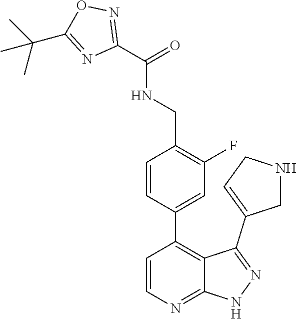 [SMILES: CC(C)(C)c1nc(C(=O)NCc2ccc(-c3ccnc4[nH]nc(C5=CCNC5)c34)cc2F)no1]

At 120° C. under N₂ atmosphere, to a stirred solution of 3-bromo-4-chloro-1-(4-methoxybenzyl)-1H-pyrazolo[3,4-b]pyridine (3.0 g, 8.53 mmol) in dioxane/H₂O (30 mL/8 mL) were added tert-butyl 3-(4,4,5,5-tetramethyl-1,3,2-dioxaborolan-2-yl)-2,5-dihydro-1H-pyrrole-1-carboxylate (2.64 g, 9.3 mmol), Pd(dppf)Cl₂.DCM (1.05 g, 1.28 mmol) and K₂CO₃ (3.52 g, 25.6 mmol). After being stirred at 100° C. for 8 hr, the reaction mixture was cooled down to room temperature and filtered through a pad of celite. The filtration was quenched with H₂O and extracted with ethyl acetate (3×). The combined organic layers were washed with brine and dried over Na₂SO₄. Solvents were removed and the residue was purified by flash chromatography (silica gel, 0˜50% ethyl acetate in petroleum ether) to provide tert-butyl 3-(4-chloro-1-(4-methoxybenzyl)-1H-pyrazolo[3,4-b]pyridin-3-yl)-2,5-dihydro-1H-pyrrole-1-carboxylate (2.4 g, 64%) as a white solid. LCMS (ESI) m/z (M/M+2): 441.36/443.36.

At 100° C. under N₂ atmosphere, to a stirred solution of tert-butyl 3-(4-chloro-1-(4-methoxybenzyl)-1H-pyrazolo[3,4-b]pyridin-3-yl)-2,5-dihydro-1H-pyrrole-1-carboxylate (2.4 g, 5.45 mmol) in dioxane/H₂O (20 mL/5 mL) were added 2,2,2-trifluoro-N-(2-fluoro-4-(4,4,5,5-tetramethyl-1,3,2-dioxaborolan-2-yl)benzyl)acetamide (2.46 g, 7.08 mmol), Pd(dppf)Cl₂.DCM (676 mg, 0.82 mmol), and Cs₂CO₃(3.55 g, 10.9 mmol). After being stirred at 100° C. overnight, the reaction mixture was cooled down to room temperature and filtered through a pad of celite. The filtration was quenched with H₂O and extracted with ethyl acetate (3×). The combined organic layers were washed with brine and dried over Na₂SO₄. Solvents were removed and the residue was purified by flash chromatography (silica gel, 0˜50% ethyl acetate in petroleum ether) tert-butyl 3-(4-(3-fluoro-4-((2,2,2-trifluoroacetamido)methyl)phenyl)-1-(4-methoxybenzyl)-1H-pyrazolo[3,4-b]pyridin-3-yl)-2,5-dihydro-1H-pyrrole-1-carboxylate (2.5 g, 74%) as a white solid. LCMS (ESI) m/z (M+1): 626.43.

At room temperature, to a stirred solution of tert-butyl 3-(4-(3-fluoro-4-((2,2,2-trifluoroacetamido)methyl)phenyl)-1-(4-methoxybenzyl)-1H-pyrazolo[3,4-b]pyridin-3-yl)-2,5-dihydro-1H-pyrrole-1-carboxylate (1.0 g, 1.6 mmol) in MeOH (10 mL) was added 1N NaOH (15 mL, 1.5 mmol). After being stirred at room temperature overnight, the reaction mixture was extracted with ethyl acetate (3×). The combined organic layers were wash with brine and dried over Na₂SO₄. Solvents were removed under vacuum to provide tert-butyl 3-(4-(4-(aminomethyl)-3-fluorophenyl)-1-(4-methoxybenzyl)-1H-pyrazolo[3,4-b]pyridin-3-yl)-2,5-dihydro-1H-pyrrole-1-carboxylate (750 mg, quant.) as a white solid. LCMS (ESI) m/z (M+1): 530.53.

At 50° C., to a stirred solution of tert-butyl 3-(4-(4-(aminomethyl)-3-fluorophenyl)-1-(4-methoxybenzyl)-1H-pyrazolo[3,4-b]pyridin-3-yl)-2,5-dihydro-1H-pyrrole-1-carboxylate (320 mg, 0.60 mmol) in DCM (10 mL) were added 5-(tert-butyl)isoxazole-3-carboxylic acid (154 mg, 0.90 mmol), HATU (344 mg, 0.90 mmol) and DIEA (178 mg, 1.8 mmol). After being stirred at room temperature overnight, the reaction mixture was quenched with saturated NaHCO₃ and extracted with ethyl acetate (3×). The combined organic layers were washed with brine and dried over Na₂SO₄. Solvents were removed under vacuum to provide tert-butyl 3-(4-(4-((5-(tert-butyl)-1,2,4-oxadiazole-3-carboxamido)methyl)-3-fluorophenyl)-1-(4-methoxybenzyl)-1H-pyrazolo[3,4-b]pyridin-3-yl)-2,5-dihydro-1H-pyrrole-1-carboxylate (300 mg, 74%) as a white solid. LCMS (ESI) m/z (M+1): 682.41.

5-(tert-butyl)-N-(4-(3-(2,5-dihydro-1H-pyrrol-3-yl)-1H-pyrazolo[3,4-b]pyridin-4-yl)-2-fluorobenzyl)-1,2,4-oxadiazole-3-carboxamide (57 mg, 27%) was obtained as a white solid following an analogous procedure to that of Example 34. LCMS (ESI) m/z (M+1): 462.45 ¹H NMR (400 MHz, DMSO) δ 14.21 (s, 1H), 9.73 (s, 2H), 9.59 (t, J=5.9 Hz, 1H), 8.64 (d, J=4.6 Hz, 1H), 7.50 (t, J=7.8 Hz, 1H), 7.36 (dd, J=10.5, 1.4 Hz, 1H), 7.31-7.20 (m, 2H), 4.59 (d, J=5.9 Hz, 2H), 4.34 (s, 2H), 4.30 (d, J=1.8 Hz, 1H), 3.82 (s, 2H), 1.44 (s, 9H)

The following compounds were prepared by analogous methods:

| Chemical structure | Chemical names | LC-MS/HNMR |
|---|---|---|
| 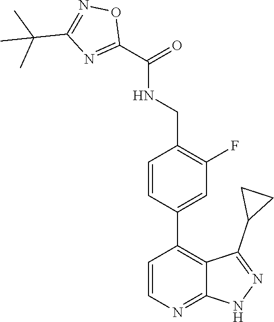 [SMILES: CC(C)(C)c1noc(C(=O)NCc2ccc(-c3ccnc4[nH]nc(C5CC5)c34)cc2F)n1] | 3-(tert-butyl)-N-(4-(3- cyclopropyl-1H-pyrazolo- [3,4-b]pyridin-4-yl)-2- fluorobenzyl)-1,2,4- oxadiazole-5-carboxamide | LCMS (ESI) m/z (M + 1): 435.46 ¹H NMR (400 MHz, DMSO) δ 13.36 (s, 1H), 9.91 (s, 1H), 8.51 (d, J = 4.7 Hz, 1H), 7.71-7.34 (m, 3H), 7.09 (d, J = 4.7 Hz, 1H), 4.60 (d, J = 5.8 Hz, 2H), 1.72-1.53 (m, 1H), 1.37 (s, 9H), 0.94-0.85 (m, 2H), 0.81-0.67 (m, 2H) |

### Example 43: (Z)-5-(tert-butyl)-N-(2-fluoro-4-(3-(pyrrolidin-3-ylidenemethyl)-1H-pyrazolo[3,4-b]pyridin-4-yl)benzyl)-1,2,4-oxadiazole-3-carboxamide

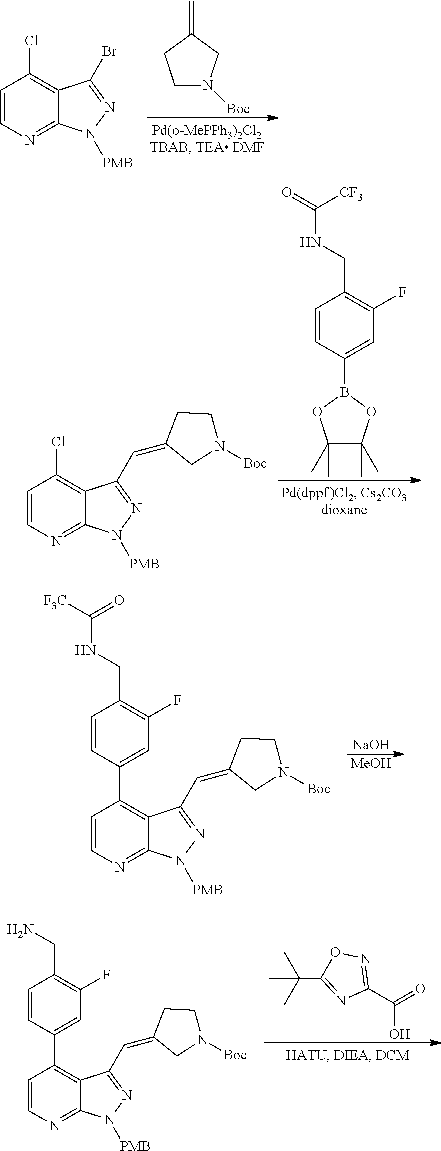

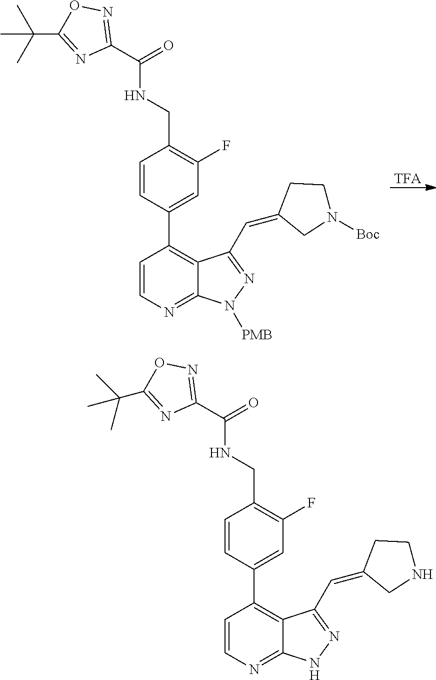

At 120° C. under N₂ atmosphere, to a stirred solution of 3-bromo-4-chloro-1-(4-methoxybenzyl)-1H-pyrazolo[3,4-b]pyridine (8.2 g, 23.2 mmol) in DMF (100 mL) were added tert-butyl 3-methylenepyrrolidine-1-carboxylate (5.3 g, 29 mmol), Pd(o-MePPh3)2Cl2 (889 mg, 1.16 mmol), TBAB (1.49 g, 4.64 mmol) and TEA (4.69 g, 46.4 mmol). After being stirred at 100° C. for 8 hr, the reaction mixture was cooled down to room temperature and filtered through a pad of celite. The filtration was quenched with H₂O and extracted with ethyl acetate (3×). The combined organic layers were washed with brine and dried over Na₂SO₄. Solvents were removed and the residue was purified by flash chromatography (silica gel, 0˜50% ethyl acetate in petroleum ether) to provide tert-butyl (Z)-3-((4-chloro-1-(4-methoxybenzyl)-1H-pyrazolo[3,4-b]pyridin-3-yl)methylene)pyrrolidine-1-carboxylate (2.4 g, 23%) as a yellow solid. LCMS (ESI) m/z (M/M+2): 455.39/457.38.

At 100° C. under N₂ atmosphere, to a stirred solution of tert-butyl (Z)-3-((4-chloro-1-(4-methoxybenzyl)-1H-pyrazolo[3,4-b]pyridin-3-yl)methylene)pyrrolidine-1-carboxylate (2.4 g, 5.27 mmol) in dioxane/H₂O (20 mL/5 mL) were added 2,2,2-trifluoro-N-(2-fluoro-4-(4,4,5,5-tetramethyl-1,3,2-dioxaborolan-2-yl)benzyl)acetamide (2.7 g, 7.9 mmol), Pd(dppf)Cl₂.DCM (860 mg, 1.0 mmol), and Cs₂CO₃ (5.15 g, 15.8 mmol). After being stirred at 100° C. overnight, the reaction mixture was cooled down to room temperature and filtered through a pad of celite. The filtration was quenched with H₂O and extracted with ethyl acetate (3×). The combined organic layers were washed with brine and dried over Na₂SO₄. Solvents were removed and the residue was purified by flash chromatography (silica gel, 0˜50% ethyl acetate in petroleum ether) (Z)-3-((4-(3-fluoro-4-((2,2,2-trifluoroacetamido)methyl)phenyl)-1-(4-methoxybenzyl)-1H-pyrazolo[3,4-b]pyridin-3-yl)methylene)pyrrolidine-1-carboxylate (2.0 g, 73%) as a yellow solid. LCMS (ESI) m/z (M+1): 640.46.

At room temperature, to a stirred solution of (Z)-3-((4-(3-fluoro-4-((2,2,2-trifluoroacetamido)methyl)phenyl)-1-(4-methoxybenzyl)-1H-pyrazolo[3,4-b]pyridin-3-yl)methylene)pyrrolidine-1-carboxylate (1.0 g, 1.56 mmol) in MeOH (10 mL) was added 4N NaOH (2 mL, 8 mmol). After being stirred at room temperature overnight, the reaction mixture was extracted with ethyl acetate (3×). The combined organic layers were washed with brine and dried over Na₂SO₄. Solvents were removed under vacuum to provide tert-butyl (Z)-3-((4-(4-(aminomethyl)-3-fluorophenyl)-1-(4-methoxybenzyl)-1H-pyrazolo[3,4-b]pyridin-3-yl)methylene)pyrrolidine-1-carboxylate (800 mg, 95%) as a yellow oil. LCMS (ESI) m/z (M+1): 544.51.

At 50° C., to a stirred solution of tert-butyl (Z)-3-((4-(4-(aminomethyl)-3-fluorophenyl)-1-(4-methoxybenzyl)-1H-pyrazolo[3,4-b]pyridin-3-yl)methylene)pyrrolidine-1-carboxylate (800 mg, 1.46 mmol) in DCM (20 mL) were added 5-(tert-butyl)isoxazole-3-carboxylic acid (289 mg, 1.7 mmol), HATU (1.1 g, 2.9 mmol) and DIEA (379 mg, 2.9 mmol). After being stirred at room temperature overnight, the reaction mixture was quenched with saturated NaHCO₃ and extracted with ethyl acetate (3×). The combined organic layers were washed with brine and dried over Na₂SO₄. Solvents were removed under vacuum to provide tert-butyl (Z)-3-((4-(4-((5-(tert-butyl)-1,2,4-oxadiazole-3-carboxamido)methyl)-3-fluorophenyl)-1-(4-methoxybenzyl)-1H-pyrazolo[3,4-b]pyridin-3-yl)methylene)pyrrolidine-1-carboxylate (540 mg, 53%) as a yellow solid. LCMS (ESI) m/z (M+1): 696.92.

At 0° C., to a stirred solution of tert-butyl (Z)-3-((4-(4-((5-(tert-butyl)-1,2,4-oxadiazole-3-carboxamido)methyl)-3-fluorophenyl)-1-(4-methoxybenzyl)-1H-pyrazolo[3,4-b]pyridin-3-yl)methylene)pyrrolidine-1-carboxylate (426 mg, 0.61 mmol) in TFA (6 mL) was added TfOH (0.5 mL). After being stirred at room temperature for 4 hr, the reaction mixture was concentrated. The residue was quenched with saturated NaHCO₃ and extracted with DCM (3×). The combined organic layers were washed with brine and dried over Na₂SO₄. Solvents were removed under vacuum and the crude product was purified by prep. HPLC (C18, 0˜90 acetonitrile in H₂O with 0.1% formic acid) to provide (Z)-5-(tert-butyl)-N-(2-fluoro-4-(3-(pyrrolidin-3-ylidenemethyl)-1H-pyrazolo[3,4-b]pyridin-4-yl)benzyl)-1,2,4-oxadiazole-3-carboxamide (22 mg, 7%) as a white solid. LCMS (ESI) m/z (M+1): 476.56. ¹H NMR (400 MHz, DMSO) δ 9.60 (d, J=6.8 Hz, 1H), 8.55 (dd, J=4.6, 2.2 Hz, 1H), 7.78-7.47 (m, 3H), 7.41 (dd, J=22.6, 8.1 Hz, 2H), 7.10 (t, J=4.5 Hz, 1H), 5.09-4.78 (d, J=6.2 Hz, 1H), 4.61 (d, J=3.9 Hz, 2H), 3.03-2.81 (m, 3H), 2.74-2.66 (m, 1H), 2.03-1.96 (m, 1H), 1.86-1.76 (m, 1H), 1.44 (s, 9H).

### Example 44: N-(4-(3-(1-acryloyl-2,5-dihydro-1H-pyrrol-3-yl)-1H-pyrazolo[3,4-b]pyridin-4-yl)-2-fluorobenzyl)-5-(tert-butyl)-1,2,4-oxadiazole-3-carboxamide

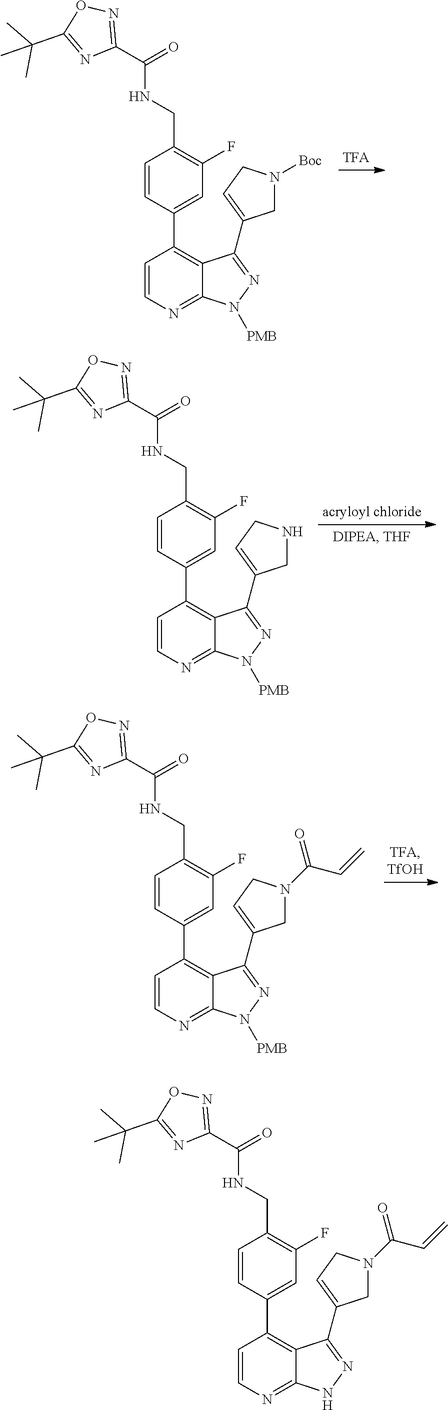

At 0° C., to a stirred solution of 5-(tert-butyl)-N-(4-(3-(2,5-dihydro-1H-pyrrol-3-yl)-1-(4-methoxybenzyl)-1H-pyrazolo[3,4-b]pyridin-4-yl)-2-fluorobenzyl)-1,2,4-oxadiazole-3-carboxamide (128 mg, 0.22 mmol) in THF (10 mL) was added DIPEA (67 mg, 0.78 mmol) followed by acryloyl chloride (15.5 mg, 0.26 mmol, in 1 mL THF). After being stirred at room temperature for 1 hr, the reaction mixture was quenched with saturated NaHCO₃ and extracted with DCM (3×). The combined organic layers were washed with brine and dried over Na₂SO₄. Solvents were removed under vacuum and the residue was purified by flash chromatography (silica gel, 0˜10% MeOH in DCM) to provide N-(4-(3-(1-acryloyl-2,5-dihydro-1H-pyrrol-3-yl)-1-(4-methoxybenzyl)-1H-pyrazolo[3,4-b]pyridin-4-yl)-2-fluorobenzyl)-5-(tert-butyl)-1,2,4-oxadiazole-3-carboxamide (67 mg, 48%) as a white solid. LCMS (ESI) m/z (M+1): 636.42.

The solution of N-(4-(3-(1-acryloyl-2,5-dihydro-1H-pyrrol-3-yl)-1-(4-methoxybenzyl)-1H-pyrazolo[3,4-b]pyridin-4-yl)-2-fluorobenzyl)-5-(tert-butyl)-1,2,4-oxadiazole-3-carboxamide (67 mg, 0.11 mmol) in TFA (3 mL) was stirred at 60° C. for 8 hr before the reaction mixture was concentrated. The residue was quenched with saturated NaHCO₃ and extracted with DCM (3×). The combined organic layers were washed with brine and dried over Na₂SO₄. Solvents were removed under vacuum and the crude product was purified by prep. HPLC (C18, 0˜90 acetonitrile in H₂O with 0.1% formic acid) to provide N-(4-(3-(1-acryloyl-2,5-dihydro-1H-pyrrol-3-yl)-1H-pyrazolo[3,4-b]pyridin-4-yl)-2-fluorobenzyl)-5-(tert-butyl)-1,2,4-oxadiazole-3-carboxamide (16 mg, 28%) as a white solid. LCMS (ESI) m/z (M+1): 516.42. ¹H NMR (400 MHz, DMSO) δ 13.98 (s, 1H), 9.57 (t, J=5.2 Hz, 1H), 8.62 (d, J=4.6 Hz, 1H), 7.53-7.43 (m, 1H), 7.36 (ddd, J=10.6, 4.1, 1.4 Hz, 1H), 7.29-7.20 (m, 2H), 6.55 (ddd, J=52.6, 16.8, 10.3 Hz, 1H), 6.18 (ddd, J=16.8, 7.8, 2.3 Hz, 1H), 5.70 (td, J=10.1, 2.3 Hz, 1H), 4.74 (s, 1H), 4.63-4.37 (m, 4H), 4.21 (s, 1H), 4.01 (s, 1H), 1.44 (d, J=3.0 Hz, 9H).

### Example 45: 5-(tert-butyl)-N-(2-fluoro-4-(3-(pyrrolidin-3-yl)-1H-pyrazolo[3,4-b]pyridin-4-yl)benzyl)-1,2,4-oxadiazole-3-carboxamide

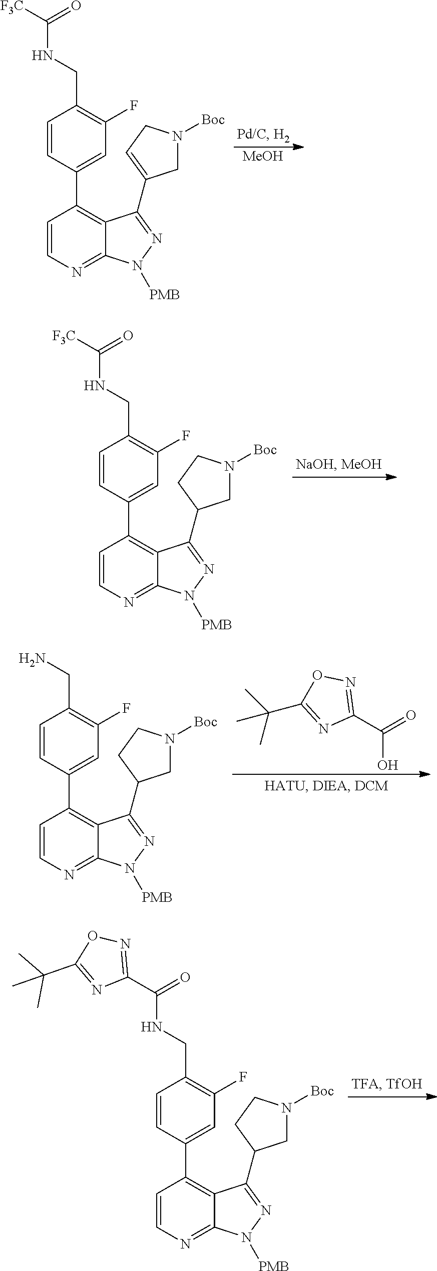

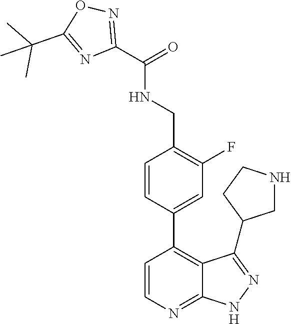 [SMILES: CC(C)(C)c1nc(C(=O)NCc2ccc(-c3ccnc4[nH]nc(C5CCNC5)c34)cc2F)no1]

At room temperature, under H2 atmosphere, to a stirred solution of tert-butyl 3-(4-(3-fluoro-4-((2,2,2-trifluoroacetamido)methyl)phenyl)-1-(4-methoxybenzyl)-1H-pyrazolo[3,4-b]pyridin-3-yl)-2,5-dihydro-1H-pyrrole-1-carboxylate (1.8 g, 2.88 mmol) in MeOH (30 mL) was added Pd/C (180 mg). After being stirred at room temperature overnight, the reaction mixture was filtered through a pad of celite. The filtrate was concentrated to provide tert-butyl 3-(4-(3-fluoro-4-((2,2,2-trifluoroacetamido)methyl)phenyl)-1-(4-methoxybenzyl)-1H-pyrazolo[3,4-b]pyridin-3-yl)pyrrolidine-1-carboxylate (780 mg, 43%) as a white solid. LCMS (ESI) m/z (M+1): 628.55.

5-(tert-butyl)-N-(2-fluoro-4-(3-(pyrrolidin-3-yl)-1H-pyrazolo[3,4-b]pyridin-4-yl)benzyl)-1,2,4-oxadiazole-3-carboxamide (35 mg, 26%) was obtained as a white solid following an analogous procedure to that of Example 34. LCMS (ESI) m/z (M+1): 464.45. ¹H NMR (400 MHz, DMSO) δ 13.77 (s, 1H), 9.58 (t, J=6.0 Hz, 1H), 9.24 (d, J=23.6 Hz, 2H), 8.57 (d, J=4.7 Hz, 1H), 7.54 (t, J=7.8 Hz, 1H), 7.48 (dd, J=10.5, 1.3 Hz, 1H), 7.40 (dd, J=7.8, 1.4 Hz, 1H), 7.12 (d, J=4.7 Hz, 1H), 4.61 (d, J=6.0 Hz, 2H), 3.58 (p, J=7.2 Hz, 1H), 3.45-3.35 (m, 1H), 3.23-3.01 (m, 3H), 1.88 (dq, J=14.1, 7.1 Hz, 1H), 1.71 (dq, J=14.3, 7.3 Hz, 1H), 1.44 (s, 9H).

The following compound was prepared by analogous methods:

| Chemical structure | Chemical names | LC-MS/HNMR |
|---|---|---|
| 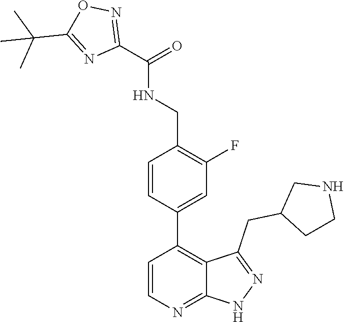 [SMILES: CC(C)(C)c1nc(C(=O)NCc2ccc(-c3ccnc4[nH]nc(CC5CCNC5)c34)cc2F)no1] | 5-(tert-butyl)-N-(2- fluoro-4-(3-(pyrrolidin- 3-ylmethyl)-1H- pyrazolo[3,4-b]pyridin- 4-yl)benzyl)-1,2,4- oxadiazole-3- carboxamide | LCMS (ESI) m/z (M + 1):478.69 ¹H NMR (400 MHz, DMSO) δ 13.74- 13.45 (m, 1H), 9.59 (t, J = 6.0 Hz, 1H), 8.87 (br, 2H), 8.54 (d, J = 4.7 Hz, 1H), 7.53 (t, J = 7.8 Hz, 1H), 7.45 (d, J = 10.6 Hz, 1H), 7.38 (d, J = 7.8 Hz, 1H), 7.10 (d, J = 4.7 Hz, 1H), 4.60 (d, J = 5.9 Hz, 2H), 3.13-3.04 (m, 2H), 3.01-2.92 (m, 1H), 2.78-2.59 (m, 3H), 2.27-2.17 (m, 1H), 1.82-1.70 (m, 1H), 1.44 (s, 9H), 1.39-1.31 (m, 1H) |

### Example 46: N-(4-(3-(1-acryloylpyrrolidin-3-yl)-1H-pyrazolo[3,4-b]pyridin-4-yl)-2-fluorobenzyl)-5-(tert-butyl)-1,2,4-oxadiazole-3-carboxamide

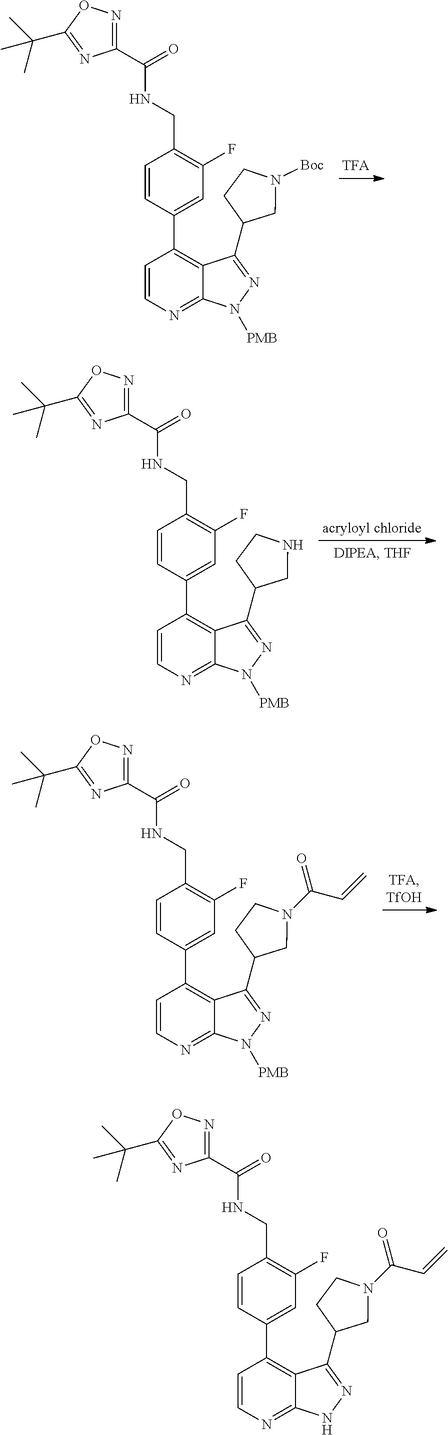

At 0° C., to a stirred solution of tert-butyl 3-(4-(4-((5-(tert-butyl)-1,2,4-oxadiazole-3-carboxamido)methyl)-3-fluorophenyl)-1-(4-methoxybenzyl)-1H-pyrazolo[3,4-b]pyridin-3-yl)pyrrolidine-1-carboxylate (200 mg, 0.29 mmol) in DCM (10 mL) was added TFA (4 mL). After being stirred at room temperature for 4 hr, the reaction mixture was concentrated. The residue was quenched with saturated NaHCO₃ and extracted with DCM (3×). The combined organic layers were washed with brine and dried over Na₂SO₄. Solvents were removed under vacuum to provide 5-(tert-butyl)-N-(2-fluoro-4-(1-(4-methoxybenzyl)-3-(pyrrolidin-3-yl)-1H-pyrazolo[3,4-b]pyridin-4-yl)benzyl)-1,2,4-oxadiazole-3-carboxamide (130 mg, 77%) as a white solid. LCMS (ESI) m/z (M+1): 584.66.

At 0° C., to a stirred solution of 5-(tert-butyl)-N-(2-fluoro-4-(1-(4-methoxybenzyl)-3-(pyrrolidin-3-yl)-1H-pyrazolo[3,4-b]pyridin-4-yl)benzyl)-1,2,4-oxadiazole-3-carboxamide (130 mg, 0.22 mmol) in THF (5 mL) was added DIPEA (85 mg, 0.66 mmol) followed by acryloyl chloride (20 mg, 0.22 mmol, in mL THF). After being stirred at room temperature for 1 hr, the reaction mixture was quenched with saturated NaHCO₃ and extracted with DCM (3×). The combined organic layers were washed with brine and dried over Na₂SO₄. Solvents were removed under vacuum and the residue was purified by flash chromatography (silica gel, 0˜10% MeOH in DCM) to provide N-(4-(3-(1-acryloylpyrrolidin-3-yl)-1-(4-methoxybenzyl)-1H-pyrazolo[3,4-b]pyridin-4-yl)-2-fluorobenzyl)-5-(tert-butyl)-1,2,4-oxadiazole-3-carboxamide (90 mg, 64%) as a white solid. LCMS (ESI) m/z (M+1): 638.50.

The solution of N-(4-(3-(1-acryloylpyrrolidin-3-yl)-1-(4-methoxybenzyl)-1H-pyrazolo[3,4-b]pyridin-4-yl)-2-fluorobenzyl)-5-(tert-butyl)-1,2,4-oxadiazole-3-carboxamide (90 mg, 0.14 mmol) in TFA (5 mL) stirred at 90° C. for 4 hr before the reaction mixture was concentrated. The residue was quenched with saturated NaHCO₃ and extracted with DCM (3×). The combined organic layers were washed with brine and dried over Na₂SO₄. Solvents were removed under vacuum and the crude product was purified by prep. HPLC (C18, 0˜90 acetonitrile in H₂O with 0.1% formic acid) to provide N-(4-(3-(1-acryloylpyrrolidin-3-yl)-1H-pyrazolo[3,4-b]pyridin-4-yl)-2-fluorobenzyl)-5-(tert-butyl)-1,2,4-oxadiazole-3-carboxamide (14 mg, 19%) as a white solid. LCMS (ESI) m/z (M+1): 518.53. ¹H NMR (400 MHz, DMSO) δ 13.60 (d, J=10.9 Hz, 1H), 9.54 (t, J=5.9 Hz, 1H), 8.55 (dd, J=4.6, 1.7 Hz, 1H), 7.61-7.45 (m, 2H), 7.41 (d, J=7.8 Hz, 1H), 7.10 (dd, J=4.6, 2.3 Hz, 1H), 6.47 (ddd, J=25.2, 16.8, 10.3 Hz, 1H), 6.07 (ddd, J=16.8, 7.3, 2.4 Hz, 1H), 5.61 (dt, J=10.4, 2.9 Hz, 1H), 4.59 (d, J=3.6 Hz, 2H), 3.73-3.15 (m, 5H), 1.94-1.66 (m, 2H), 1.43 (s, 9H)

The following compound was prepared by analogous methods:

| Chemical structure | Chemical names | LC-MS/HNMR |
|---|---|---|
| 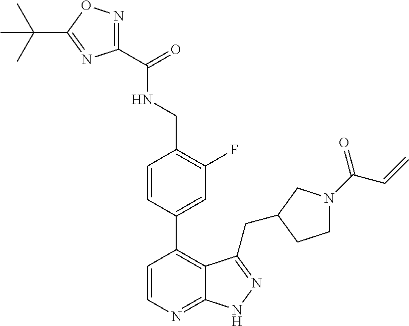 [SMILES: C=CC(=O)N1CCC(Cc2n[nH]c3nccc(-c4ccc(CNC(=O)c5noc(C(C)(C)C)n5)c(F)c4)c23)C1] | N-(4-(3-((1- acryloylpyrrolidin-3- yl)methyl)-1H- pyrazolo[3,4-b]pyridin-4- yl)-2-fluorobenzyl)-5- (tert-butyl)-1,2,4- oxadiazole-3- carboxamide | LCMS (ESI) m/z (M + 1): 532.68 ¹H NMR (400 MHz, DMSO) δ 13.54 (s, 1H), 9.55 (t, J = 6.0 Hz, 1H), 8.53 (d, J = 4.6 Hz, 1H), 7.53 (q, J = 7.7 Hz, 1H), 7.45 (t, J = 9.0 Hz, 1H), 7.36 (d, 1H), 7.12-6.98 (m, 1H), 6.43 (ddd, J = 19.4, 16.8, 10.3 Hz, 1H), 6.06 (ddd, J = 16.8, 5.7, 2.5 Hz, 1H), 5.59 (ddd, J = 12.6, 10.4, 2.5 Hz, 1H), 4.59 (d, J = 6.0 Hz, 2H), 3.31-3.07 (m, 2H), 2.99-2.80 (m, 1H), 2.74-2.59 (m, 2H), 2.18- 1.97 (m, 2H), 1.74-1.62 (m, 1H), 1.38 (s, 9H), 1.32-1.27 (m, 1H) |

### Example 47: (R)-5-(tert-butyl)-N-(1-(4-(3-(1-(dimethylcarbamoyl)-1,2,5,6-tetrahydropyridin-3-yl)-1H-pyrazol[3,4-b]pyridin-4-yl)phenyl)ethyl)-1,2,4-oxadiazole-3-carboxamide

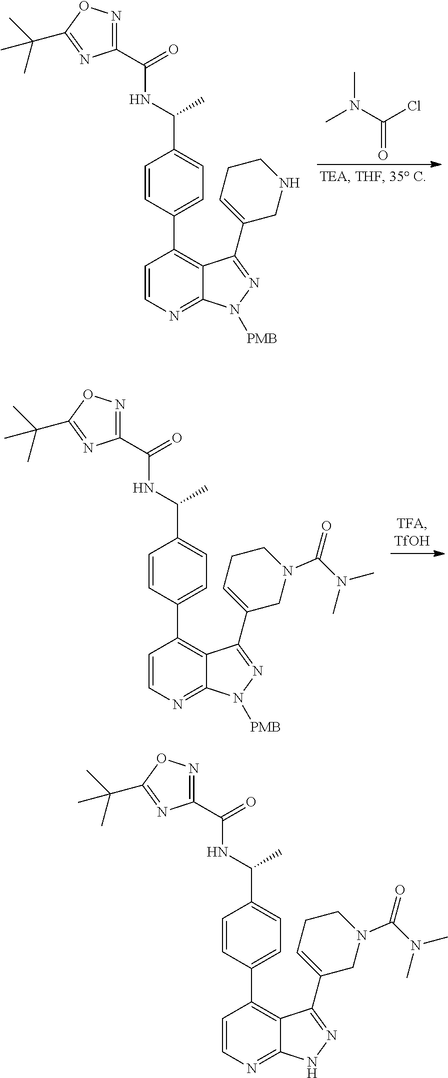

(R)-3-(tert-butyl)-N-(1-(4-(3-(1-(dimethylcarbamoyl)piperidin-4-yl)-1H-pyrazolo[3,4-b]pyridin-4-yl)phenyl)ethyl)-1,2,4-oxadiazole-5-carboxamide (18 mg, 10% over 2 steps) was obtained as white solid following an analogous procedure to that of Example 37. LCMS (ESI) m/z (M+1): 543.46. ¹H NMR (400 MHz, DMSO) δ 13.78 (s, 1H), 9.89 (d, J=8.0 Hz, 1H), 8.57 (d, J=4.7 Hz, 1H), 7.45 (dd, J=21.1, 8.2 Hz, 4H), 7.17 (d, J=4.7 Hz, 1H), 5.18 (dd, J=14.7, 7.2 Hz, 1H), 4.88 (s, 1H), 4.13-3.85 (m, 2H), 3.06-2.95 (m, 2H), 2.72 (s, 6H), 1.74-1.61 (m, 2H), 1.56 (d, J=7.1 Hz, 3H), 1.37 (s, 9H)

The following compound was prepared by analogous methods:

| Chemical structure | Chemical names | LC-MS/HNMR |
|---|---|---|
| 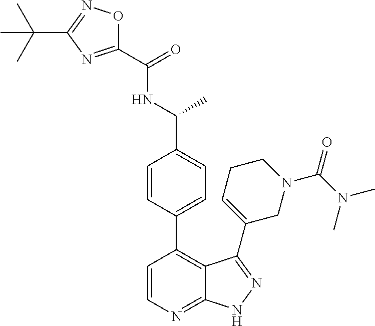 [SMILES: C[C@@H](NC(=O)c1nc(C(C)(C)C)no1)c1ccc(-c2ccnc3[nH]nc(C4=CCCN(C(=O)N(C)C)C4)c23)cc1] | (R)-3-(tert-butyl)-N-(1- (4-(3-(1- (dimethylcarbamoyl)- 1,2,5,6-tetrahydropyridin- 3-yl)-1H-pyrazolo[3,4- b]pyridin-4- yl)phenyl)ethyl)-1,2,4- oxadiazole-5- carboxamide | LCMS (ESI) m/z (M + 1): 543.58 ¹H NMR (400 MHz, DMSO) δ 13.78 (s, 1H), 9.89 (d, J = 8.0 Hz, 1H), 8.57 (d, J = 4.7 Hz, 1H), 7.45 (dd, J = 21.1, 8.2 Hz, 4H), 7.17 (d, J = 4.7 Hz, 1H), 5.18 (dd, J = 14.7, 7.2 Hz, 1H), 4.88 (s, 1H), 4.13-3.85 (m, 2H), 3.06-2.95 (m, 2H), 2.72 (s, 6H), 1.74-1.61 (m, 2H), 1.56 (d, J = 7.1 Hz, 3H), 1.37 (s, 9H) |

### Example 48: (R)-3-(tert-butyl)-N-(1-(4-(3-(1-(dimethylcarbamoyl)piperidin-4-yl)-1H-pyrazolo[3,4-b]pyridin-4-yl)phenyl)ethyl)-1,2,4-oxadiazole-5-carboxamide

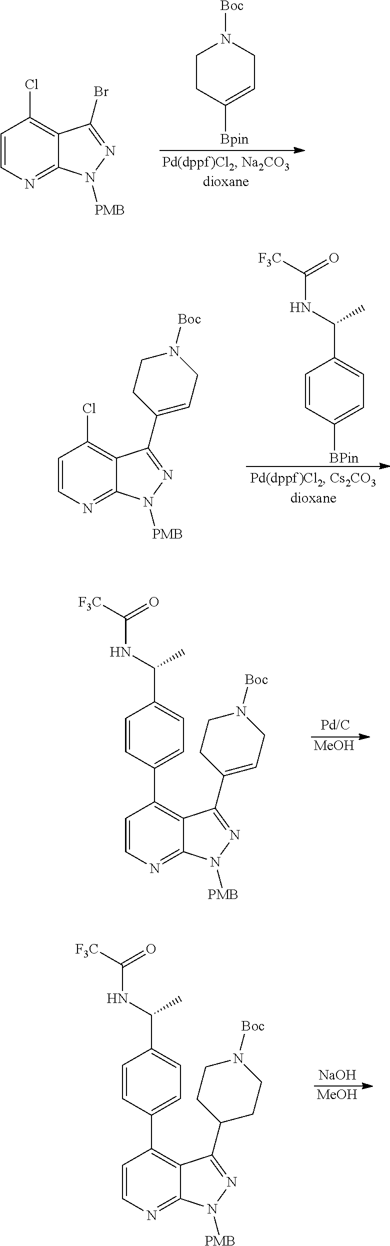

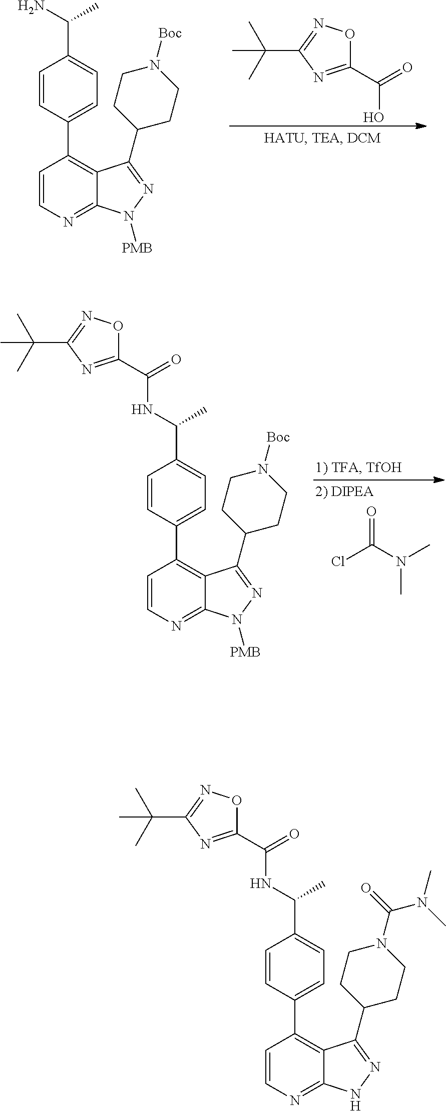

tert-butyl (R)-4-(1-(4-methoxybenzyl)-4-(4-(1-(2,2,2-trifluoroacetamido)ethyl)phenyl)-1H-pyrazolo[3,4-b]pyridin-3-yl)-3,6-dihydropyridine-1(2H)-carboxylate (1.4 g, 83%) was obtained as yellow solid following an analogous procedure to that of Example 43. LCMS (ESI) m/z (M+1): 636.54.

(R)-3-(tert-butyl)-N-(1-(4-(3-(1-(dimethylcarbamoyl)piperidin-4-yl)-1H-pyrazolo[3,4-b]pyridin-4-yl)phenyl)ethyl)-1,2,4-oxadiazole-5-carboxamide (70 mg, 30%) was obtained as a white solid following an analogous procedure to that of Example 37. LCMS (ESI) m/z (M+1): 545.57. ¹H NMR (400 MHz, DMSO) δ 13.43 (s, 1H), 9.90 (d, J=8.0 Hz, 1H), 8.50 (d, J=4.6 Hz, 1H), 7.55 (dd, J=32.6, 8.2 Hz, 4H), 7.05 (d, J=4.6 Hz, 1H), 5.38-5.08 (m, 1H), 3.58-3.18 (m, 4H), 2.65 (s, 6H), 2.34-2.24 (m, 1H), 1.59 (d, J=7.0 Hz, 3H), 1.52-1.42 (m, 4H), 1.36 (s, 9H).

### Example 49: (R)—N-((5-(tert-butyl)-1,2,4-oxadiazol-3-yl)methyl)-2-fluoro-4-(3-(pyrrolidin-3-ylamino)-1H-pyrazolo[3,4-b]pyridin-4-yl)benzamide and (R)-4-(3-((1-acryloylpyrrolidin-3-yl)amino)-1H-pyrazolo[3,4-b]pyridin-4-yl)-N-((5-(tert-butyl)-1,2,4-oxadiazol-3-yl)methyl)-2-fluorobenzamide

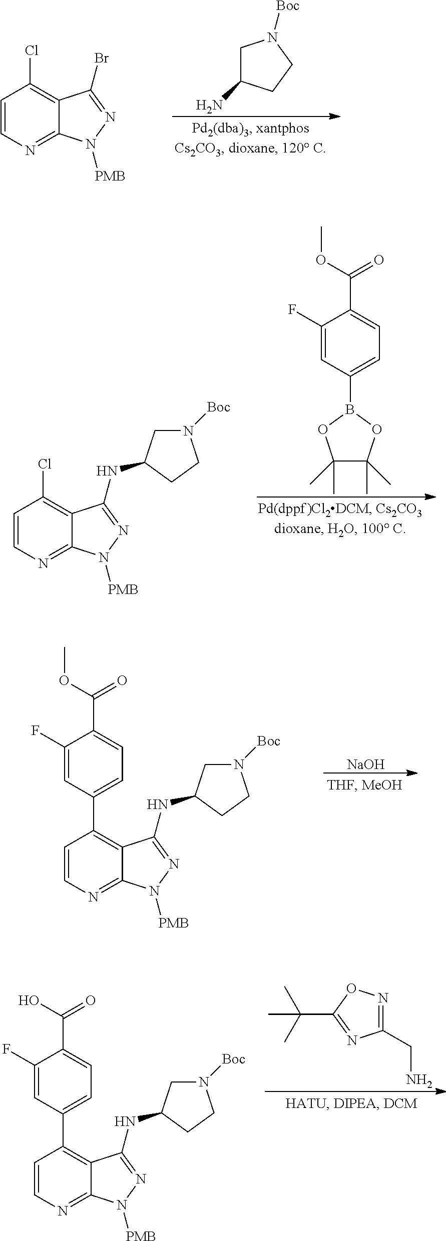

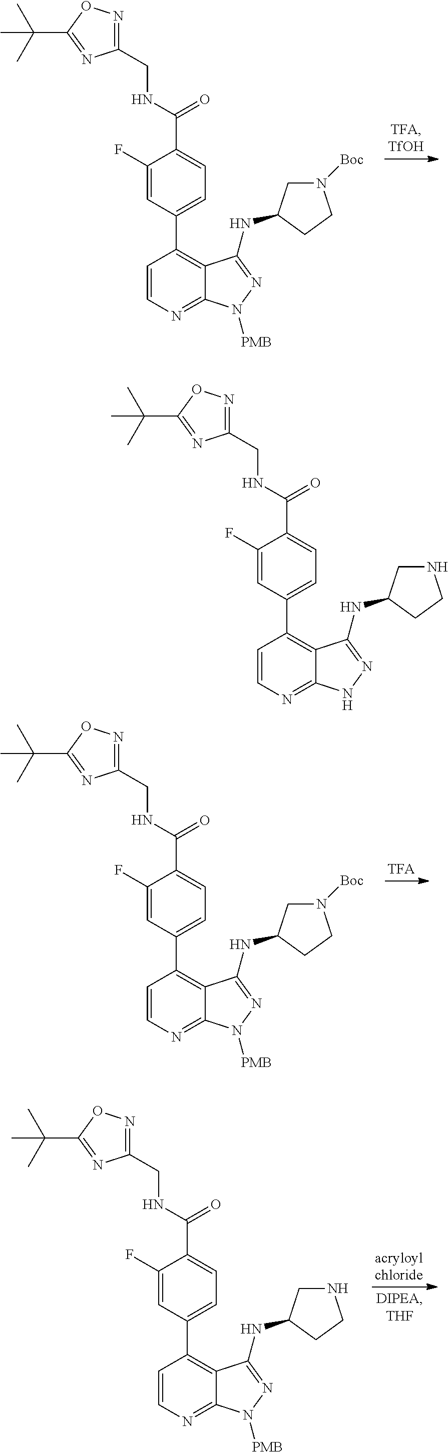

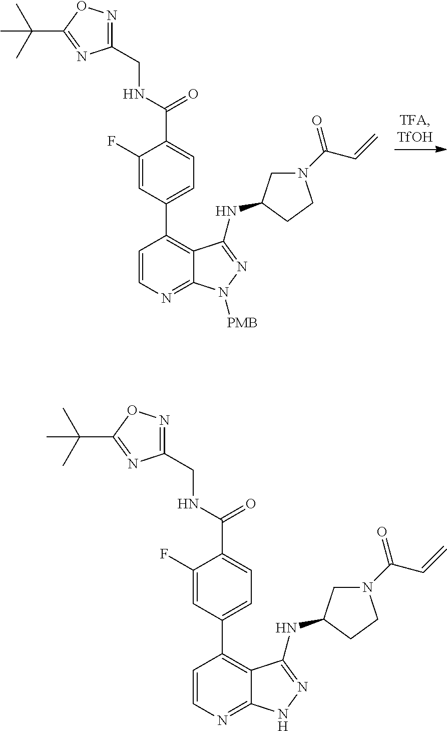

(R)-4-(3-((1-(tert-butoxycarbonyl)pyrrolidin-3-yl)amino)-1-(4-methoxybenzyl)-1H-pyrazolo[3,4-b]pyridin-4-yl)-2-fluorobenzoic acid (3.4 g, 70%) was obtained as a yellow solid following an analogous procedure to that of Example 34. LCMS (ESI) m/z (M+1): 562.57.

(R)—N-((5-(tert-butyl)-1,2,4-oxadiazol-3-yl)methyl)-2-fluoro-4-(3-(pyrrolidin-3-ylamino)-1H-pyrazolo[3,4-b]pyridin-4-yl)benzamide HCl salt (38 mg, 35%) was obtained as a red solid following an analogous procedure to that of Example 34. LCMS (ESI) m/z (M+1): 479.37. ¹H NMR (400 MHz, DMSO) δ 12.74 (s, 1H), 9.19 (d, J=21.6 Hz, 2H), 9.01 (dd, J=8.5, 5.7 Hz, 1H), 8.49 (d, J=4.8 Hz, 1H), 7.85 (t, J=7.8 Hz, 1H), 7.78-7.57 (m, 2H), 7.09 (d, J=4.8 Hz, 1H), 4.94 (s, 1H), 4.61 (d, J=5.8 Hz, 2H), 4.31-4.26 (m, 1H), 3.41-3.17 (m, 4H), 2.17 (td, J=14.5, 8.0 Hz, 1H), 1.90 (dt, J=17.6, 6.4 Hz, 1H), 1.39 (s, 9H).

(R)-4-(3-((1-acryloylpyrrolidin-3-yl)amino)-1H-pyrazolo[3,4-b]pyridin-4-yl)-N-((5-(tert-butyl)-1,2,4-oxadiazol-3-yl)methyl)-2-fluorobenzamide (44 mg, 50%) was obtained as yellow solid following an analogous procedure to that of Example 35. LCMS (ESI) m/z (M+1): 533.38. ¹H NMR (400 MHz, DMSO) δ 12.64 (s, 1H), 8.97 (s, 1H), 8.47 (d, J=4.7 Hz, 1H), 7.80 (t, J=7.7 Hz, 1H), 7.57 (ddd, J=14.0, 10.8, 6.5 Hz, 2H), 7.05 (d, J=4.7 Hz, 1H), 6.54 (ddd, J=37.9, 16.8, 10.3 Hz, 1H), 6.11 (ddd, J=16.8, 8.3, 2.4 Hz, 1H), 5.63 (ddd, J=20.9, 10.3, 2.4 Hz, 1H), 4.71 (d, J=5.2 Hz, 1H), 4.61 (d, J=5.8 Hz, 2H), 4.28-4.12 (m, 1H), 3.87-3.60 (m, 2H), 3.54-3.37 (m, 2H), 2.23-2.08 (m, 1H), 1.96-1.79 (m, 1H), 1.39 (s, 9H).

### Example 50: (R)—N-(1-(4-(4-(aminomethyl)-3-fluorophenyl)-1H-pyrazolo[3,4-b]pyridin-3-yl)pyrrolidin-3-yl)acetamide

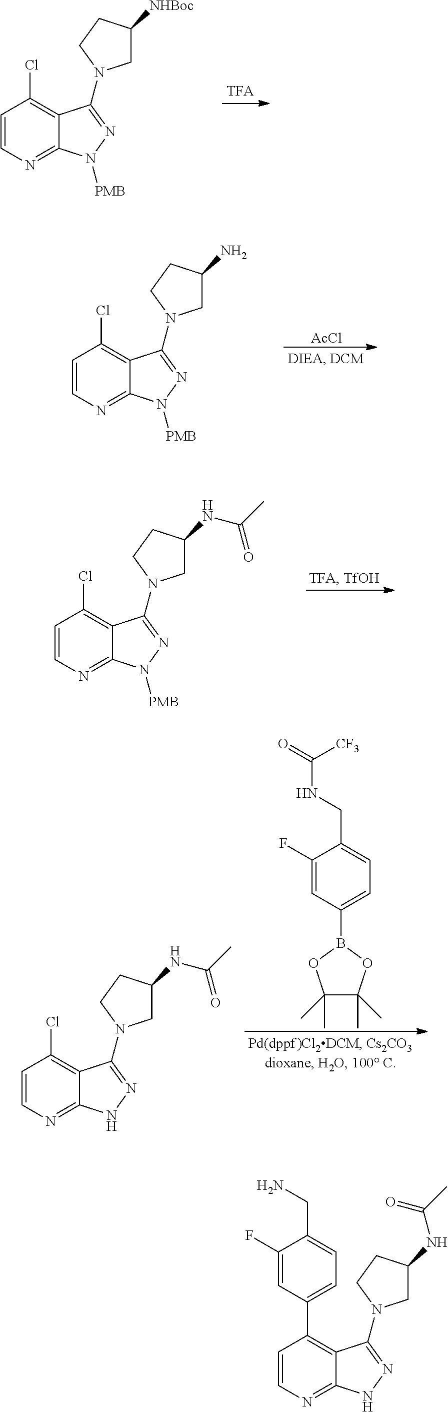

At 100° C., to a stirred solution of tert-butyl (R)-(1-(4-chloro-1-(4-methoxybenzyl)-1H-pyrazolo[3,4-b]pyridin-3-yl)pyrrolidin-3-yl)carbamate (45.0 g, 98.4 mmol) in DCM (400 mL) was added TFA (150 mL). After being stirred at room temperature for 4 hr, the reaction mixture was concentrated. The residue was quenched with saturated NaHCO₃ and extracted with DCM (3×). The combined organic layers were washed with brine and dried over Na₂SO₄. Solvents were removed under vacuum to provide (R)-1-(4-chloro-1-(4-methoxybenzyl)-1H-pyrazolo[3,4-b]pyridin-3-yl)pyrrolidin-3-amine as a yellow solid which was used in the next step directly.

At 0° C., to a stirred solution of (R)-1-(4-chloro-1-(4-methoxybenzyl)-1H-pyrazolo[3,4-b]pyridin-3-yl)pyrrolidin-3-amine in THF (600 mL) was added DIPEA (38 g, 295.2 mmol) followed by acetyl chloride (7.7 g, 98.4 mmol, in 20 mL THF). After being stirred at room temperature for 1 hr, the reaction mixture was quenched with saturated NaHCO₃ and extracted with DCM (3×). The combined organic layers were washed with brine and dried over Na₂SO₄. Solvents were removed under vacuum and the residue was purified by flash chromatography (silica gel, 0˜10% MeOH in DCM) to provide (R)—N-(1-(4-chloro-1-(4-methoxybenzyl)-1H-pyrazolo[3,4-b]pyridin-3-yl)pyrrolidin-3-yl)acetamide (29.6 g, 75%) as a yellow solid. LCMS (ESI) m/z (M/M+2): 400.20/402.19.

At 0° C., to a stirred solution of (R)—N-(1-(4-chloro-1-(4-methoxybenzyl)-1H-pyrazolo[3,4-b]pyridin-3-yl)pyrrolidin-3-yl)acetamide (29.0 g, 72.5 mmol) in TFA (100 mL) was added TfOH (200 mL). After being stirred at room temperature overnight, the reaction mixture was concentrated and the residue was partitioned between DCM and H₂O, quenched with saturated NaHCO₃. The layers were separated and the aqueous layer was extracted with DCM (3×). The combined organic layers were washed with brine and dried over Na₂SO₄. Solvents were removed under vacuum and the crude product was dissolved in dioxane/H₂O (200 mL/20 mL) followed by introduction of 2,2,2-trifluoro-N-(2-fluoro-4-(4,4,5,5-tetramethyl-1,3,2-dioxaborolan-2-yl)benzyl)acetamide (30 g, 87 mmol) followed by Pd(dppf)Cl2.DCM (5.99 g, 7.25 mmol) and Cs₂CO₃ (47.2 g, 145 mmol). After purged with N2 (3×), the reaction mixture was heated to 100° C. for 24 hr before cooled down to room temperature and filtered through a pad of celite. The filtrate was concentrated and purified by flash chromatography (silica gel, 0-10% MeOH in DCM) to provide (R)—N-(1-(4-(4-(aminomethyl)-3-fluorophenyl)-1H-pyrazolo[3,4-b]pyridin-3-yl)pyrrolidin-3-yl)acetamide (12.0 g, 80%) as a yellow solid. LCMS (ESI) m/z (M+1): 369.20. ¹H NMR (400 MHz, DMSO) δ 8.45 (d, J=4.6 Hz, 1H), 7.93 (d, J=6.7 Hz, 1H), 7.65 (t, J=7.7 Hz, 1H), 7.47 (t, J=10.2 Hz, 2H), 7.06 (d, J=4.6 Hz, 1H), 5.76 (s, 1H), 4.09 (dd, J=12.5, 6.2 Hz, 1H), 3.85 (s, 2H), 3.08 (dd, J=9.7, 7.1 Hz, 1H), 2.89 (dd, J=16.0, 7.7 Hz, 1H), 2.78 (dd, J=14.6, 8.2 Hz, 1H), 2.69 (dd, J=9.9, 5.8 Hz, 1H), 1.97-1.87 (m, 1H), 1.77 (s, 3H), 1.52 (dt, J=11.3, 5.8 Hz, 1H).

### Example 51: (R)—N-(4-(3-((1-acryloylpyrrolidin-3-yl)amino)-1H-pyrazolo[3,4-b]pyridin-4-yl)-2-fluorobenzyl)methanesulfonamide

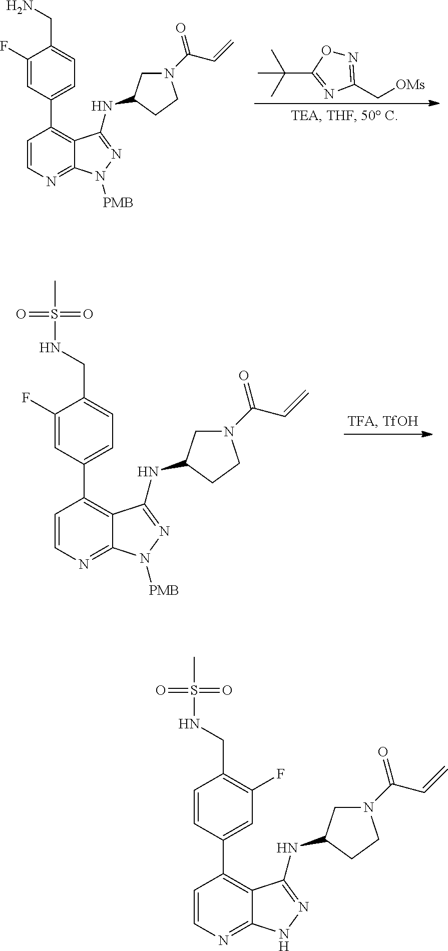

(R)-1-(3-((4-(4-(aminomethyl)-3-fluorophenyl)-1-(4-methoxybenzyl)-1H-pyrazolo[3,4-b]pyridin-3-yl)amino)pyrrolidin-1-yl)prop-2-en-1-one (50 mg, quant.) was obtained as yellow oil following an analogous procedure to that of Example 37. LCMS (ESI) m/z (M+1): 501.34.

At 50° C., to a stirred solution of (R)-1-(3-((4-(4-(aminomethyl)-3-fluorophenyl)-1-(4-methoxybenzyl)-1H-pyrazolo[3,4-b]pyridin-3-yl)amino)pyrrolidin-1-yl)prop-2-en-1-one (50 mg, 0.1 mmol) in THF (2 mL) was added (5-(tert-butyl)-1,2,4-oxadiazol-3-yl)methyl methanesulfonate (25 mg, 0.1 mmol) followed by TEA (20 mg, 0.2 mmol). After being stirred at 50° C. overnight, the reaction mixture was cooled down to room temperature and quenched with H₂O, extracted with ethyl acetate. The organic layer was washed with brine and dried over Na₂SO₄. Solvent was removed under vacuum and the residue was purified by flash chromatography (silica gel, 0˜10% MeOH in DCM) to provide (R)—N-(4-(3-((1-acryloylpyrrolidin-3-yl)amino)-1-(4-methoxybenzyl)-1H-pyrazolo[3,4-b]pyridin-4-yl)-2-fluorobenzyl)methanesulfonamide (20 mg, 33%) as a yellow oil. LCMS (ESI) m/z (M+1): 597.32.

At 0° C., to a stirred solution of (R)—N-(4-(3-((1-acryloylpyrrolidin-3-yl)amino)-1-(4-methoxybenzyl)-1H-pyrazolo[3,4-b]pyridin-4-yl)-2-fluorobenzyl)methanesulfonamide (20 mg) in TFA (1 mL) was added TfOH (0.3 mL). After being stirred at room temperature for 4 hr, the reaction mixture was concentrated. The residue was quenched with saturated NaHCO₃ and extracted with DCM (3×). The combined organic layers were washed with brine and dried over Na₂SO₄. Solvents were removed under vacuum and the crude product was purified by prep. HPLC (C18, 0˜90 acetonitrile in H₂O with 0.1% formic acid) to provide (R)—N-(4-(3-((1-acryloylpyrrolidin-3-yl)amino)-1H-pyrazolo[3,4-b]pyridin-4-yl)-2-fluorobenzyl)methanesulfonamide (1.6 mg) as a yellow solid. LCMS (ESI) m/z (M+1): 459.39.

¹H NMR (400 MHz, DMSO) δ 12.60 (s, 1H), 8.45 (d, J=4.7 Hz, 1H), 7.70 (s, 1H), 7.60 (t, J=7.3 Hz, 1H), 7.52-7.42 (m, 2H), 7.01 (d, J=4.7 Hz, 1H), 6.63-6.47 (m, 1H), 6.13 (ddd, J=16.8, 8.8, 2.4 Hz, 1H), 5.65 (ddd, J=20.5, 10.3, 2.4 Hz, 1H), 4.53 (t, J=4.7 Hz, 1H), 4.30 (d, J=5.1 Hz, 2H), 4.17 (dd, J=33.3, 5.3 Hz, 1H), 3.88-3.58 (m, 2H), 3.50-3.42 (m, 2H), 2.95 (d, J=2.1 Hz, 3H), 2.21-2.09 (m, 1H), 2.02-1.90 (m, 1H)

The following compound was prepared by analogous methods:

| Chemical structure | Chemical names | LC-MS/HNMR |
|---|---|---|
| 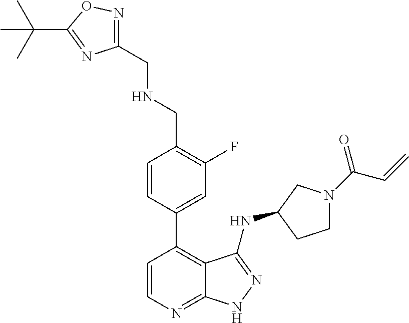 [SMILES: C=CC(=O)N1CC[C@@H](Nc2n[nH]c3nccc(-c4ccc(CNCc5noc(C(C)(C)C)n5)c(F)c4)c23)C1] | (R)-1-(3-((4-(4-((((5-(tert- butyl)-1,2,4-oxadiazol-3- yl)methyl)amino)methyl)- 3-fluorophenyl)-1H- pyrazolo[3,4-b]pyridin-3- yl)amino)pyrrolidin-1- yl)prop-2-en-1-one | LCMS (ESI) m/z (M + 1): 519.51 ¹H NMR (400 MHz, DMSO) δ 12.57 (s, 1H), 8.44 (d, J = 4.8 Hz, 1H), 7.60 (t, J = 7.7 Hz, 1H), 7.49- 7.35 (m, 2H), 7.00 (d, J = 4.8 Hz, 1H), 6.62-6.44 (m, 2H), 6.11 (ddd, J = 16.8, 8.4, 2.4 Hz, 1H), 5.67- 5.58 (m, 1H), 4.50 (t, J = 5.2 Hz, 1H), 4.23- 4.11 (m, 1H), 3.92-3.80 (m, 4H), 3.66-3.55 (m, 2H), 3.48-3.41 (m, 2H), 2.17-2.08 (m, 1H), 1.95-1.84 (m, 1H), 1.38 (s, 9H) |

### Example 52: 3-(tert-butyl)-N-(2-fluoro-4-(3-(2-oxopyrrolidin-1-yl)-1H-pyrazolo[3,4-b]pyridin-4-yl)benzyl)-1,2,4-oxadiazole-5-carboxamide

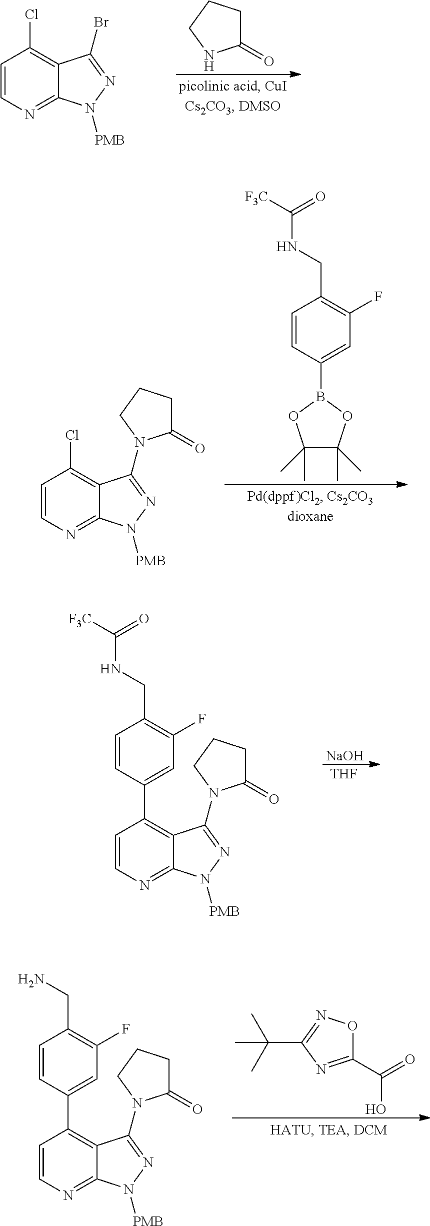

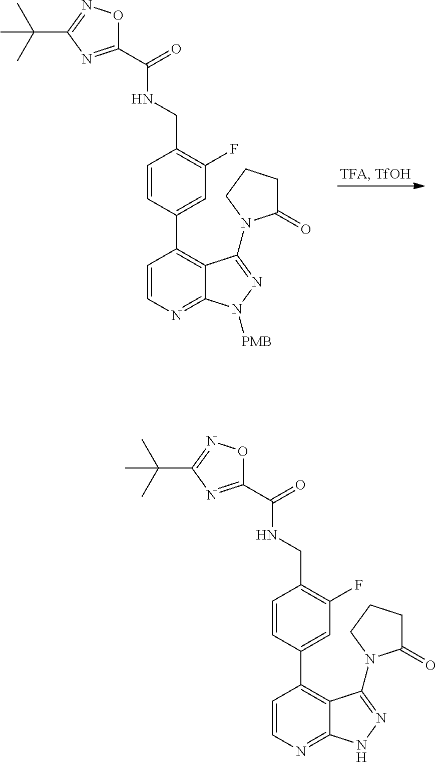

At 120° C. under N₂ atmosphere, to a stirred solution of 3-bromo-4-chloro-1-(4-methoxybenzyl)-1H-pyrazolo[3,4-b]pyridine (3.0 g, 8.54 mmol) in DMSO (30 mL) were added pyrrolidin-2-one (729 mg, 8.54 mmol), CuI (651 mg, 3.42 mmol), picolinic acid (420 mg, 3.42 mmol) and Cs₂CO₃ (5.57 g, 17.08 mmol). After being stirred at 120° C. for 24 hr, the reaction mixture was cooled down to room temperature and filtered through a pad of celite. The filtration was quenched with H₂O and extracted with ethyl acetate (3×). The combined organic layers were washed with brine and dried over Na₂SO₄. Solvents were removed and the residue was purified by flash chromatography (silica gel, 0˜50% ethyl acetate in petroleum ether) to provide 1-(4-chloro-1-(4-methoxybenzyl)-1H-pyrazolo[3,4-b]pyridin-3-yl)pyrrolidin-2-one (1.4 g, 47%) as a yellow solid. LCMS (ESI) m/z (M/M+2): 356.89/358.61.

Synthesis of 3-(tert-butyl)-N-(2-fluoro-4-(3-(2-oxopyrrolidin-1-yl)-1H-pyrazolo[3,4-b]pyridin-4-yl)benzyl)-1,2,4-oxadiazole-5-carboxamide (24 mg, 7% over 2 steps) was following an analogous procedure to that of Example 34. LCMS (ESI) m/z (M+1): 478.28. ¹H NMR (400 MHz, DMSO) δ 13.82 (s, 1H), 9.95 (t, J=6.0 Hz, 1H), 8.60 (d, J=4.7 Hz, 1H), 7.53 (t, J=7.9 Hz, 1H), 7.36-7.25 (m, 2H), 7.23 (d, J=4.7 Hz, 1H), 4.58 (d, J=6.0 Hz, 2H), 3.66 (t, J=6.8 Hz, 2H), 2.04 (t, J=7.7 Hz, 2H), 1.96-1.86 (m, 2H), 1.37 (s, 9H).

### Example 53: (R)-3-(tert-butyl)-N-(2-fluoro-4-(3-(3-(3-methyl-2-oxotetrahydropyrimidin-1(2H)-yl)pyrrolidin-1-yl)-1H-pyrazolo[3,4-b]pyridin-4-yl)benzyl)-1,2,4-oxadiazole-5-carboxamide

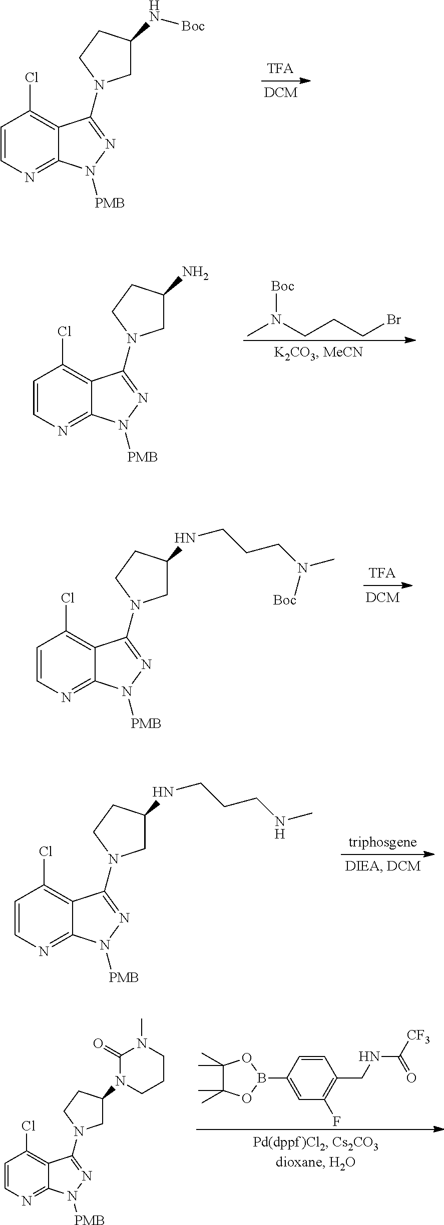

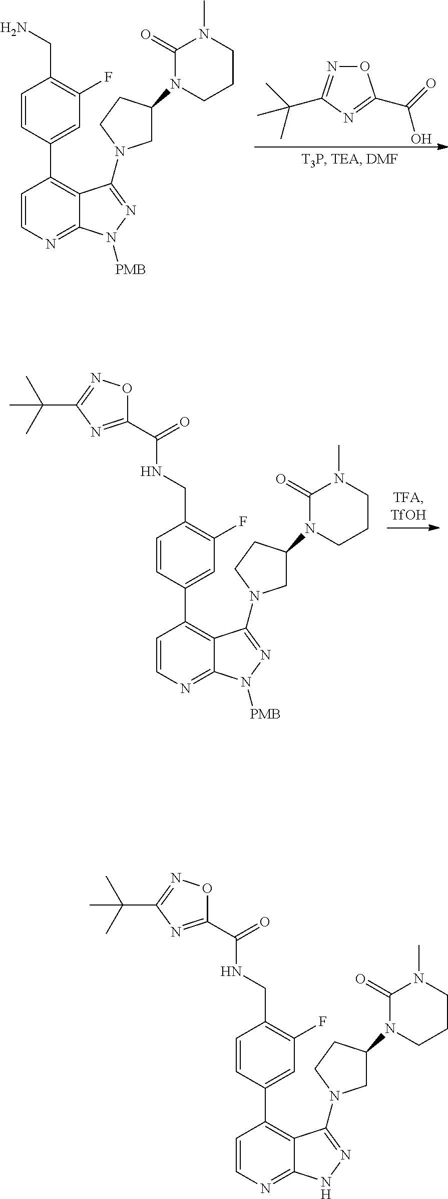

(R)-1-(4-chloro-1-(4-methoxybenzyl)-1H-pyrazolo[3,4-b]pyridin-3-yl)pyrrolidin-3-amine was obtained as a yellow solid by treating tert-butyl (R)-(1-(4-chloro-1-(4-methoxybenzyl)-1H-pyrazolo[3,4-b]pyridin-3-yl)pyrrolidin-3-yl)carbamate with TFA.

At room temperature under N₂ atmosphere, to a stirred solution of tert-butyl (R)-(1-(4-chloro-1-(4-methoxybenzyl)-1H-pyrazolo[3,4-b]pyridin-3-yl)pyrrolidin-3-yl)carbamate (1.5 g, 4.19 mmol) in MeCN (20 mL) were added K₂CO₃ (1.738 g, 12.58 mmol) and tert-butyl (3-bromopropyl)(methyl)carbamate (1.59 g, 6.29 mmol). After being stirred at room temperature overnight, the reaction mixture was quenched with H₂O and extracted with ethyl acetate (3×). The combined organic layers were washed with brine and dried over Na₂SO₄. Solvents were removed under vacuum and the residue was purified by flash chromatography (silica gel, 0˜10% MeOH in DCM) to provide tert-butyl (R)-(3-((1-(4-chloro-1-(4-methoxybenzyl)-1H-pyrazolo[3,4-b]pyridin-3-yl)pyrrolidin-3-yl)amino)propyl)(methyl)carbamate (830 mg, 37%) as a yellow solid. LCMS (ESI) m/z (M/M+2): 529.24/531.76.

(R)—N1-(1-(4-chloro-1-(4-methoxybenzyl)-1H-pyrazolo[3,4-b]pyridin-3-yl)pyrrolidin-3-yl)-N3-methylpropane-1,3-diamine (650 mg, 97%) was obtained as a yellow solid by treating tert-butyl (R)-(3-((1-(4-chloro-1-(4-methoxybenzyl)-1H-pyrazolo[3,4-b]pyridin-3-yl)pyrrolidin-3-yl)amino)propyl)(methyl)carbamate (830 mg, 1.57 mmol) with TFA. LCMS (ESI) m/z (M/M+2): 429.16/431.48.

At 0° C. under N₂ atmosphere, to a stirred solution of (R)—N1-(1-(4-chloro-1-(4-methoxybenzyl)-1H-pyrazolo[3,4-b]pyridin-3-yl)pyrrolidin-3-yl)-N3-methylpropane-1,3-diamine (800 mg, 1.87 mmol) in DCM (30 mL) was added DIEA (1 mL) followed by triphosgene (205 mg, 0.69 mmol, in 2 mL DCM). After being stirred at room temperature overnight, the reaction mixture was quenched with saturated NaHCO₃. The layers were separated and the organic layers were washed with brine, dried over Na₂SO₄. Solvents were removed under vacuum and the residue was purified by flash chromatography (silica gel, 0˜50% ethyl acetate in petroleum ether) to provide (R)-1-(1-(4-chloro-1-(4-methoxybenzyl)-1H-pyrazolo[3,4-b]pyridin-3-yl)pyrrolidin-3-yl)-3-methyltetrahydropyrimidin-2(1H)-one (464 mg, 67%) as a yellow solid. LCMS (ESI) m/z (M/M+2): 455.41/457.52.

At 100° C. under N₂ atmosphere, to a stirred solution of (R)-1-(1-(4-chloro-1-(4-methoxybenzyl)-1H-pyrazolo[3,4-b]pyridin-3-yl)pyrrolidin-3-yl)-3-methyltetrahydropyrimidin-2(1H)-one (464 mg, 1.02 mmol) in dioxane/H₂O (8 mL/3 mL) were added 2,2,2-trifluoro-N-(2-fluoro-4-(4,4,5,5-tetramethyl-1,3,2-dioxaborolan-2-yl)benzyl)acetamide (532 mg, 1.53 mmol), Pd(dppf)Cl₂.DCM (84 mg, 0.1 mmol), and Cs₂CO₃ (998 mg, 3.06 mmol). After being stirred at 100° C. overnight, the reaction mixture was cooled down to room temperature and filtered through a pad of celite. The filtration was quenched with H₂O and extracted with ethyl acetate (3×). The combined organic layers were washed with brine and dried over Na₂SO₄. Solvents were removed and the residue was purified by flash chromatography (silica gel, 0˜50% ethyl acetate in petroleum ether) to provide (R)-1-(1-(4-(4-(aminomethyl)-3-fluorophenyl)-1-(4-methoxybenzyl)-1H-pyrazolo[3,4-b]pyridin-3-yl)pyrrolidin-3-yl)-3-methyltetrahydropyrimidin-2(1H)-one (240 mg, 43%) as a yellow solid. LCMS (ESI) m/z (M+1): 544.80.

At 50° C., to a stirred solution of (R)-1-(1-(4-(4-(aminomethyl)-3-fluorophenyl)-1-(4-methoxybenzyl)-1H-pyrazolo[3,4-b]pyridin-3-yl)pyrrolidin-3-yl)-3-methyltetrahydropyrimidin-2(1H)-one (100 mg, 0.184 mmol) in DMF (5 mL) were added 3-(tert-butyl)-1,2,4-oxadiazole-5-carboxylic acid (47 mg, 0.276 mmol), T3P (351 mg, 0.552 mmol) and TEA (0.13 mL, 0.92 mmol). After being stirred at room temperature overnight, the reaction mixture was quenched with saturated NaHCO₃ and extracted with ethyl acetate (3×). The combined organic layers were washed with brine and dried over Na₂SO₄. Solvents were removed under vacuum to (R)-3-(tert-butyl)-N-(2-fluoro-4-(1-(4-methoxybenzyl)-3-(3-(3-methyl-2-oxotetrahydropyrimidin-1(2H)-yl)pyrrolidin-1-yl)-1H-pyrazolo[3,4-b]pyridin-4-yl)benzyl)-1,2,4-oxadiazole-5-carboxamide (50 mg, 39%) as a yellow solid. LCMS (ESI) m/z (M+1): 697.92.

At 0° C., to a stirred solution of (R)-3-(tert-butyl)-N-(2-fluoro-4-(1-(4-methoxybenzyl)-3-(3-(3-methyl-2-oxotetrahydropyrimidin-1(2H)-yl)pyrrolidin-1-yl)-1H-pyrazolo[3,4-b]pyridin-4-yl)benzyl)-1,2,4-oxadiazole-5-carboxamide (50 mg) in TFA (5 mL) was added TfOH (0.5 mL). After being stirred at room temperature for 4 hr, the reaction mixture was concentrated. The residue was quenched with saturated NaHCO₃ and extracted with DCM (3×). The combined organic layers were washed with brine and dried over Na₂SO₄. Solvents were removed under vacuum and the crude product was purified by prep. HPLC (C18, 0˜90 acetonitrile in H₂O with 0.1% formic acid) to provide (R)-3-(tert-butyl)-N-(2-fluoro-4-(3-(3-(3-methyl-2-oxotetrahydropyrimidin-1(2H)-yl)pyrrolidin-1-yl)-1H-pyrazolo[3,4-b]pyridin-4-yl)benzyl)-1,2,4-oxadiazole-5-carboxamide (29 mg, 70%) as a white solid. LCMS (ESI) m/z (M+1): 576.43. ¹H NMR (400 MHz, DMSO) δ 12.87 (s, 1H), 9.94 (t, J=6.0 Hz, 1H), 8.47 (d, J=4.7 Hz, 1H), 7.66-7.39 (m, 3H), 7.08 (d, J=4.7 Hz, 1H), 5.03-4.82 (m, 1H), 4.61 (dd, J=15.4, 6.1 Hz, 2H), 3.28-3.07 (m, 4H), 3.05 (dd, J=10.4, 8.6 Hz, 1H), 2.82-2.76 (m, 1H), 2.75 (s, 3H), 2.70-2.61 (m, 1H), 1.86-1.69 (m, 4H), 1.52-1.42 (m, 1H), 1.36 (s, 9H)

The following compound was prepared by analogous methods:

| Chemical structure | Chemical names | LC-MS/HNMR |
|---|---|---|
| 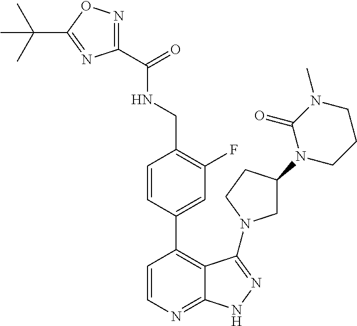 [SMILES: CN1CCCN([C@@H]2CCN(c3n[nH]c4nccc(-c5ccc(CNC(=O)c6noc(C(C)(C)C)n6)c(F)c5)c34)C2)C1=O] | (R)-5-(tert-butyl)-N-(2- fluoro-4-(3-(3-(3-methyl- 2- oxotetrahydropyrimidin- 1(2H)-yl)pyrrolidin-1-yl)- 1H-pyrazolo[3,4- b]pyridin-4-yl)benzyl)- 1,2,4-oxadiazole-3- carboxamide | LCMS (ESI) m/z (M + 1): 576.47 ¹H NMR (400 MHz, DMSO) δ 12.86 (s, 1H), 9.55 (t, J = 6.0 Hz, 1H), 8.47 (d, J = 4.7 Hz, 1H), 7.64- 7.38 (m, 3H), 7.08 (d, J = 4.7 Hz, 1H), 5.04-4.89 (m, 1H), 4.74- 4.48 (m, 2H), 3.22-3.08 (m, 4H), 3.07-3.01 (m, 1H), 2.83-2.78 (m, 1H), 2.75 (s, 3H), 2.68-2.61 (m, 1H), 2.59-2.52 (m, 1H), 1.87-1.69 (m, 3H), 1.55-1.45 (m, 1H), 1.43 (s, 9H) |

### Example 54: (2S,5R)-5-((4-(3-fluoro-4-((1-oxo-3,4,6,7,8,9-hexahydropyrazino[1,2-a]indol-2(1H)-yl)methyl)phenyl)-1H-pyrazolo[3,4-b]pyridin-3-yl)amino)-N,N,2-trimethylpiperidine-1-carboxamide

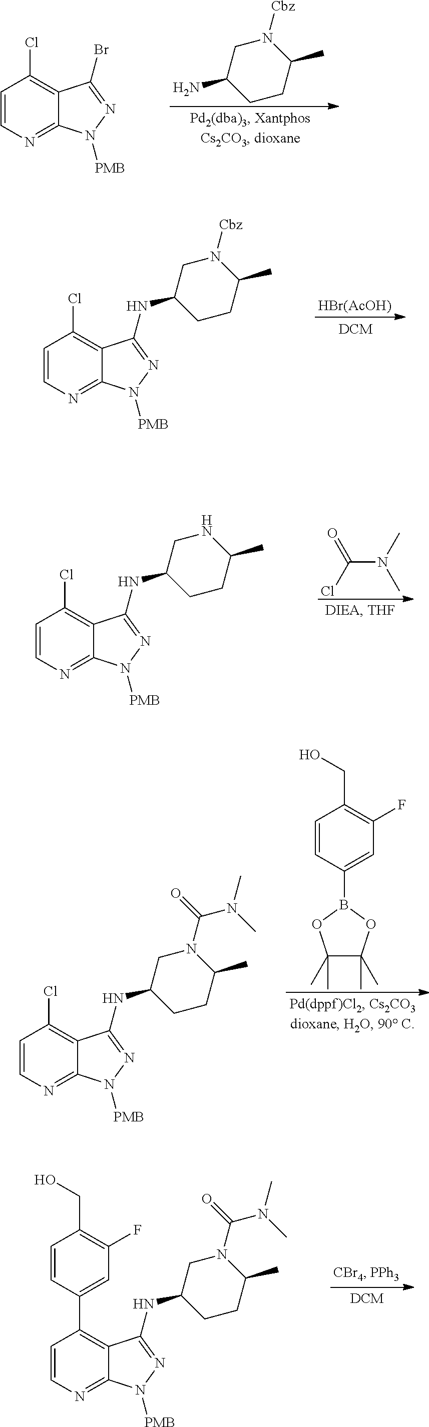

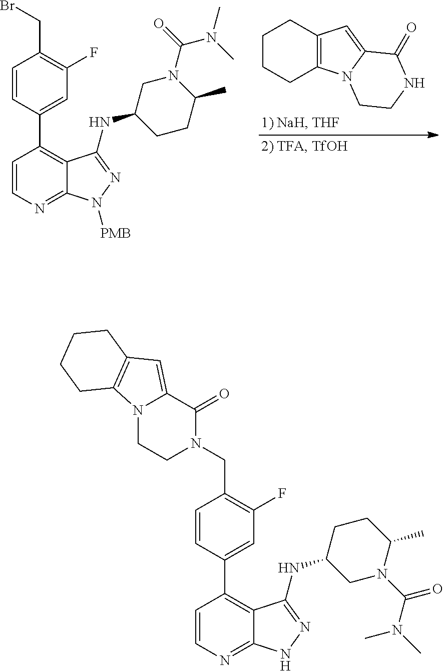

At 120° C. under N₂ atmosphere, to a stirred solution of 3-bromo-4-chloro-1-(4-methoxybenzyl)-1H-pyrazolo[3,4-b]pyridine (10.0 g, 28 mmol) in dioxane (200 mL) were added benzyl (2S,5R)-5-amino-2-methylpiperidine-1-carboxylate (7.7 g, 30.8 mmol), Pd₂(dba)₃ (2.6 g, 2.8 mmol), xantphos (3.3 g, 5.6 mmol) and Cs₂CO₃ (27 g, 84 mmol). After being stirred at 120° C. for 8 hr, the reaction mixture was cooled down to room temperature and filtered through a pad of celite. The filtration was quenched with H₂O and extracted with ethyl acetate (3×). The combined organic layers were washed with brine and dried over Na₂SO₄. Solvents were removed and the residue was purified by flash chromatography (silica gel, 0˜50% ethyl acetate in petroleum ether) to provide benzyl (2S,5R)-5-((4-chloro-1-(4-methoxybenzyl)-1H-pyrazolo[3,4-b]pyridin-3-yl)amino)-2-methylpiperidine-1-carboxylate (7.38 g, 50%) as a yellow solid. LCMS (ESI) m/z (M/M+2): 386.06/388.37.

At 0° C., to a stirred solution of benzyl (2S,5R)-5-((4-chloro-1-(4-methoxybenzyl)-1H-pyrazolo[3,4-b]pyridin-3-yl)amino)-2-methylpiperidine-1-carboxylate (2.5 g, 4.8 mmol) in DCM (2 mL) was added HBr/AcOH (6 mL, 33%). After being stirred at room temperature for 4 hr, the reaction mixture was quenched with saturated NaHCO₃ and extracted with DCM. The organic layer was washed with brine and dried over Na₂SO₄. Solvents were removed under vacuum to provide 4-chloro-1-(4-methoxybenzyl)-N-((3R,6S)-6-methylpiperidin-3-yl)-1H-pyrazolo[3,4-b]pyridin-3-amine (1.0 g, 52%) as a yellow oil. LCMS (ESI) m/z (M/M+2): 386.06/388.37.

At 35° C., to a stirred solution of 4-chloro-1-(4-methoxybenzyl)-N-((3R,6S)-6-methylpiperidin-3-yl)-1H-pyrazolo[3,4-b]pyridin-3-amine (1.0 g, 2.5 mmol) in THF (10 mL) was added DIEA (645 mg, 5 mmol) followed by dimethylcarbamic chloride (800 mg, 7.5 mmol, in THF). After being stirred at room temperature for 1 hr, the reaction mixture was quenched with saturated NaHCO₃ and extracted with DCM (3×). The combined organic layers were washed with brine and dried over Na₂SO₄. Solvents were removed under vacuum and the residue was purified by flash chromatography (silica gel, 0˜10% MeOH in DCM) to (2S,5R)-5-((4-chloro-1-(4-methoxybenzyl)-1H-pyrazolo[3,4-b]pyridin-3-yl)amino)-N,N,2-trimethylpiperidine-1-carboxamide (1.0 g, 85%) as a yellow oil. LCMS (ESI) m/z (M/M+2): 457.46/459.40.

(2S,5R)-5-((4-(3-fluoro-4-((1-oxo-3,4,6,7,8,9-hexahydropyrazino[1,2-a]indol-2(1H)-yl)methyl)phenyl)-1H-pyrazolo[3,4-b]pyridin-3-yl)amino)-N,N,2-trimethylpiperidine-1-carboxamide (53 mg, 44%) was obtained as a white solid following an analogous procedure to that of Example 26.

The following compound was prepared by analogous methods:

| Chemical structure | Chemical names | LC-MS/HNMR |
|---|---|---|
| 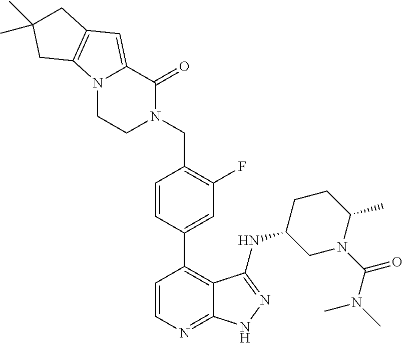 [SMILES: C[C@H]1CC[C@@H](Nc2n[nH]c3nccc(-c4ccc(CN5CCn6c(cc7c6CC(C)(C)C7)C5=O)c(F)c4)c23)CN1C(=O)N(C)C] | (2S,5R)-5-((4-(4-((7,7- dimethyl-1-oxo- 1,3,4,6,7,8-hexahydro- 2H- cyclopenta[4,5]pyrrolo[1, 2-a]pyrazin-2-yl)methyl)- 3-fluorophenyl)-1H- pyrazolo[3,4-b]pyridin-3- yl)amino)-N,N,2- trimethylpiperidine-1- carboxamide | LCMS (ESI) m/z (M + 1): 613.84 ¹H NMR (400 MHz, DMSO) δ 12.46 (s, 1H), 8.42 (d, J = 4.6 Hz, 1H), 7.67-7.34 (m, 3H), 6.97 (d, J = 4.9 Hz, 1H), 6.44 (s, 1H), 4.77 (s, 2H), 4.22 (d, J = 7.4 Hz, 1H), 4.14-3.94 (m, 2H), 3.68 (d, J = 7.6 Hz, 3H), 3.58-3.47 (m, 4H), 2.66 (s, 6H), 1.80-1.69 (m, 2H), 1.59 (dd, J = 18.2, 9.7 Hz, 2H), 1.49-1.38 (m, 2H), 1.18 (s, 6H), 1.02 (d, J = 6.6 Hz, 3H) |

### Example 55: 3-(tert-butyl)-N-(2-fluoro-4-(3-(((3R,6S)-6-methylpiperidin-3-yl)amino)-1H-pyrazolo[3,4-b]pyridin-4-yl)benzyl)-1,2,4-oxadiazole-5-carboxamide

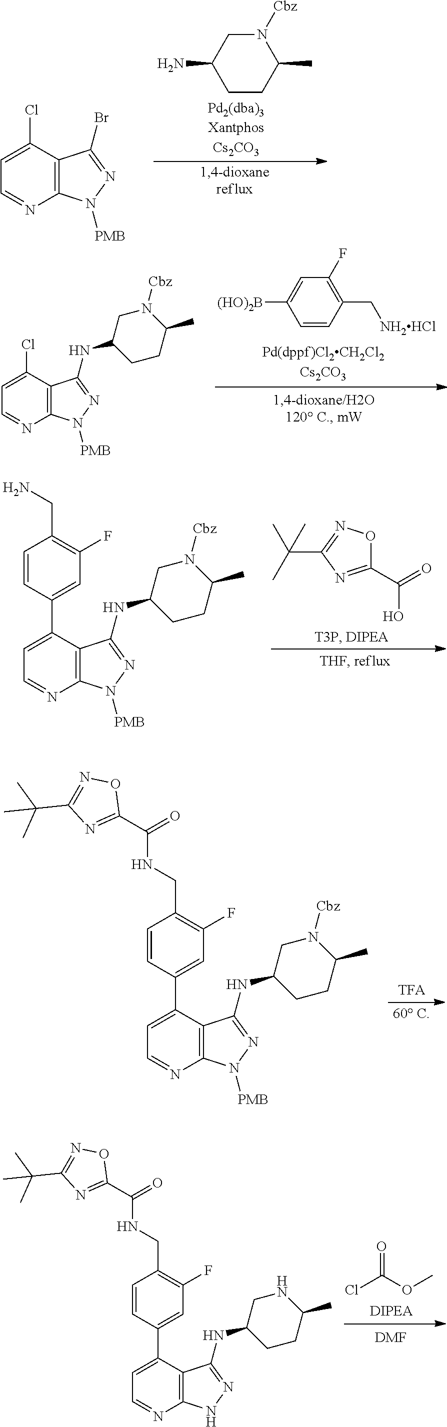

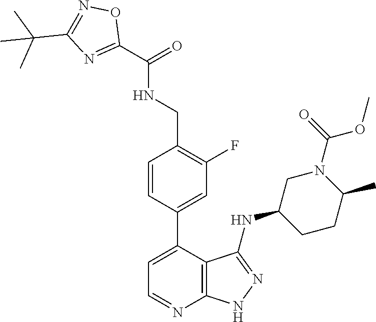 [SMILES: COC(=O)N1C[C@H](Nc2n[nH]c3nccc(-c4ccc(CNC(=O)c5nc(C(C)(C)C)no5)c(F)c4)c23)CC[C@@H]1C]

In a 250 mL round-bottomed flask with reflux condenser outfitted with a nitrogen inlet adapter, 3-bromo-4-chloro-1-(4-methoxybenzyl)-1H-pyrazolo[3,4-b]pyridine (3 g, 8.51 mmol), (2S,5R)-benzyl 5-amino-2-methylpiperidine-1-carboxylate (3.17 g, 12.76 mmol), Pd₂(dba)₃ (tris(dibenzylideneacetone)dipalladium(0)) (1.558 g, 1.702 mmol), (9,9-dimethyl-9H-xanthene-4,5-diyl)bis(diphenylphosphine), cesium carbonate (8.32 g, 25.5 mmol) and dioxane (85 mL) were added to give an orange suspension. The suspension was degassed with bubbling N2 and then heated at 130° C. for 3 h. The reaction mixture was filtered through a pad of celite washing with ethyl acetate. Remove solvent under reduced pressure. The crude material was purified via chromatography (10%-30% EtOAc/Hexanes over 30 min; 80 g Redi-Sep silica gel column) to furnish (2S,5R)-benzyl 5-((4-chloro-1-(4-methoxybenzyl)-1H-pyrazolo[3,4-b]pyridin-3-yl)amino)-2-methylpiperidine-1-carboxylate (2.7 g, 61% yield) as a yellow foam. LC-MS (ESI): m/z (M+1) 521.4.

In two 20 mL microwave reaction vials, (2S,5R)-benzyl 5-((4-chloro-1-(4-methoxybenzyl)-1H-pyrazolo[3,4-b]pyridin-3-yl)amino)-2-methylpiperidine-1-carboxylate (2.701 g, 5.19 mmol), (4-(aminomethyl)-3-fluorophenyl)boronic acid, hydrochloric acid (1.600 g, 7.79 mmol), Pd(dppf)Cl₂—CH₂Cl₂ (0.424 g, 0.519 mmol), and cesium carbonate (5.08 g, 15.58 mmol) in dioxane (36 ml)/water (3.6 ml) were added to give an orange suspension. Each suspension was heated in a Biotage® microwave at 120° C. for 3 h. The reaction mixture was transferred to a separatory funnel, washing with ethyl acetate and water. The aqueous layer was extracted with ethyl acetate (3×50 mL). The combined organic layers were washed with brine, dried over Na₂SO₄, filtered, and concentrated under reduced pressure. The crude material was purified via chromatography (0%-20% iPrOH/CH2Cl2 w/0.1% Et3N over 20 min; 80 g Redi-Sep silica gel column) to afford (2S,5R)-benzyl 5-((4-(4-(aminomethyl)-3-fluorophenyl)-1-(4-methoxybenzyl)-1H-pyrazolo[3,4-b]pyridin-3-yl)amino)-2-methylpiperidine-1-carboxylate (2.87 g, 91% yield) as a brown foam. LC-MS (ESI): m/z (M+1) 609.8.

In a 100 mL round-bottomed flask with reflux condenser, (2S,5R)-benzyl 5-((4-(4-(aminomethyl)-3-fluorophenyl)-1-(4-methoxybenzyl)-1H-pyrazolo[3,4-b]pyridin-3-yl)amino)-2-methylpiperidine-1-carboxylate (1.463 g, 2.403 mmol), 3-(tert-butyl)-1,2,4-oxadiazole-5-carboxylic acid (0.613 g, 3.61 mmol), T3P (50% ethyl acetate solution) (2.83 ml, 4.81 mmol), and DIPEA (2.099 ml, 12.02 mmol) in tetrahydrofuran (THF) (24 ml) were added to give a brown solution. The solution was heated at reflux for 45 min. The reaction was quenched with methanol and poured onto saturated NaHCO₃. The aqueous layer was extracted with ethyl acetate (3×40 mL). The combined organic layers were dried over Na₂SO₄, filtered through a disposable fritted filter funnel and concentrated. The crude material was purified via chromatography (0%-100% EtOAc/Hexanes over 30 min; 40 g Redi-Sep silica gel column) to afford (2S,5R)-benzyl 5-((4-(4-((3-(tert-butyl)-1,2,4-oxadiazole-5-carboxamido)methyl)-3-fluorophenyl)-1-(4-methoxybenzyl)-1H-pyrazolo[3,4-b]pyridin-3-yl)amino)-2-methylpiperidine-1-carboxylate (665 mg, 36% yield) as a yellow foam. LC-MS (ESI): m/z (M+1) 762.2.

In a 40 mL scintillation vial, (2S,5R)-benzyl 5-((4-(4-((3-(tert-butyl)-1,2,4-oxadiazole-5-carboxamido)methyl)-3-fluorophenyl)-1-(4-methoxybenzyl)-1H-pyrazolo[3,4-b]pyridin-3-yl)amino)-2-methylpiperidine-1-carboxylate (665 mg, 0.874 mmol) was dissolved in trifluoroacetic acid (TFA) (10 ml), stirred and heated to 60° C. over night. The solvent was removed under reduced pressure to afford a brown residue. The residual TFA was quenched with several drops of Et3N. The crude material was purified via chromatography (0%-20% iPrOH/CH2Cl2 w/0.1% Et3N over 20 min; 24 g Redi-Sep® silica gel column) to afford 3-(tert-butyl)-N-(2-fluoro-4-(3-(((3R,6S)-6-methylpiperidin-3-yl)amino)-1H-pyrazolo[3,4-b]pyridin-4-yl)benzyl)-1,2,4-oxadiazole-5-carboxamide (318 mg, 72% yield) as an orange oil. LC-MS (ESI): m/z (M+1) 507.6.

N,N-diisopropylethylamine (0.047 ml, 0.269 mmol) and methyl chloroformate (0.021 ml, 0.269 mmol) were added sequentially to a solution of 3-(tert-butyl)-N-(2-fluoro-4-(3-(((3R,6S)-6-methylpiperidin-3-yl)amino)-1H-pyrazolo[3,4-b]pyridin-4-yl)benzyl)-1,2,4-oxadiazole-5-carboxamide (68.1 mg, 0.134 mmol) in dimethyl formamide (1 ml). After 10 minutes, the reaction was quenched with several drops formic acid, the reaction mixture was diluted with water/methanol/DMSO and submitted for prep HPLC (40% to 60% MeCN/water with 0.1% FMA). The desired fractions were submitted for lyophilization to afford (2S,5R)-methyl 5-((4-(4-((3-(tert-butyl)-1,2,4-oxadiazole-5-carboxamido)methyl)-3-fluorophenyl)-1H-pyrazolo[3,4-b]pyridin-3-yl)amino)-2-methylpiperidine-1-carboxylate (16.3 mg, 19% yield) as a yellow solid. LC-MS (ESI): m/z (M+1) 565.9.

The following compounds were prepared by analogous methods:

| Chemical Structure | Chemical Name | LC-MS, ¹H NMR |
|---|---|---|
| 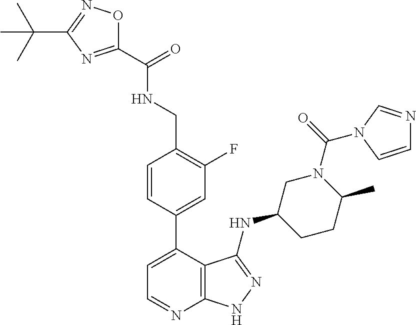 [SMILES: C[C@H]1CC[C@@H](Nc2n[nH]c3nccc(-c4ccc(CNC(=O)c5nc(C(C)(C)C)no5)c(F)c4)c23)CN1C(=O)n1ccnc1] | N-(4-(3-(((3R,6S)-1-(1H- imidazole-1-carbonyl)-6- methylpiperidin-3- yl)amino)-1H- pyrazolo[3,4-b]pyridin-4- yl)-2-fluorobenzyl)-3- (tert-butyl)-1,2,4- oxadiazole-5- carboxamide | LC-MS (ESI): m/z (M + 1) 601.4 ¹H NMR (400 MHz, DMSO- d₆) δ 12.29 (s, 1H), 9.56 (s, 1H), 8.42 (d, J = 4.7 Hz, 1H), 7.93 (d, J = 1.2 Hz, 1H), 7.62 (t, J = 7.9 Hz, 1H), 7.49-7.40 (m, 4H), 7.00 (t, J = 1.2 Hz, 1H), 6.96 (d, J = 4.7 Hz, 1H), 4.64 (d, J = 5.3 Hz, 2H), 4.23 (t, J = 6.5 Hz, 1H), 4.12 (dd, J = 13.1, 4.5 Hz, 1H), 4.03 (d, J = 7.3 Hz, 1H), 3.69-3.54 (m, 1H), 1.91-1.74 (m, 2H), 1.64-1.50 (m, 2H), 1.39 (s, 9H), 1.23 (d, J = 6.8 Hz, 3H). |
| 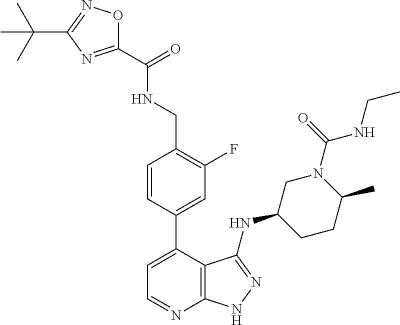 [SMILES: CCNC(=O)N1C[C@H](Nc2n[nH]c3nccc(-c4ccc(CNC(=O)c5nc(C(C)(C)C)no5)c(F)c4)c23)CC[C@@H]1C] | 3-(tert-butyl)-N-(4-(3- (((3R,6S)-1- (ethylcarbamoyl)-6- methylpiperidin-3- yl)amino)-1H- pyrazolo[3,4-b]pyridin-4- yl)-2-fluorobenzyl)-1,2,4- oxadiazole-5- carboxamide | LC-MS (ESI) : m/z (M + 1) 578.4 ¹H NMR (400 MHz, DMSO- d₆) δ 12.25 (s, 1H), 9.57 (s, 1H), 8.42 (d, J = 4.7 Hz, 1H), 7.92 (d, J = 7.4 Hz, 1H), 7.63 (t, J = 7.9 Hz, 1H), 7.49-7.38 (m, 3H), 6.95 (d, J = 4.7 Hz, 1H), 5.97 (s, 1H), 4.71-4.58 (m, 2H), 4.20 (t, J = 6.6 Hz, 1H), 4.07 (dd, J = 12.8, 4.6 Hz, 1H), 3.83 (d, J = 7.5 Hz, 1H), 3.35 (dt, J = 11.7, 5.6 Hz, 1H), 2.41 (dd, J = 12.8, 10.8 Hz, 1H), 1.79 (d, J = 12.4 Hz, 1H), 1.60 (ddt, J = 13.6, 9.7, 4.9 Hz, 1H), 1.50-1.41 (m, 1H), 1.39 (s, 9H), 1.36-1.31 (m, 0H), 1.03 (dd, J = 8.0, 6.8 Hz, 6H). |
| 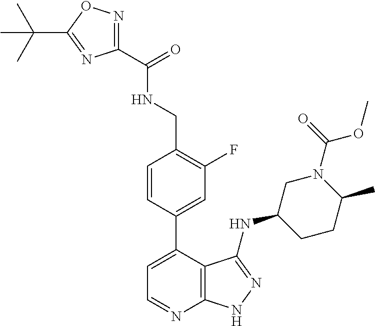 [SMILES: COC(=O)N1C[C@H](Nc2n[nH]c3nccc(-c4ccc(CNC(=O)c5noc(C(C)(C)C)n5)c(F)c4)c23)CC[C@@H]1C] | (2S,5R)-methyl 5-((4-(4- ((5-(tert-butyl)-1,2,4- oxadiazole-3- carboxamido)methyl)-3- fluorophenyl)-1H- pyrazolo[3,4-b]pyridin-3- yl)amino)-2- methylpiperidine-1- carboxylate | LC-MS (ESI): m/z (M + 1) 565.8 ¹H NMR (400 MHz, DMSO-d₆) δ 12.24 (s, 1H), 9.12 (s, 1H), 8.42 (d, J = 4.7 Hz, 1H), 7.60 (t, J = 7.9 Hz, 1H), 7.43 (dd, J = 5.3, 1.6 Hz, 1H), 7.41 (d, J = 2.2 Hz, 1H), 6.95 (d, J = 4.7 Hz, 1H), 4.64 (d, J = 6.0 Hz, 2H), 4.30- 4.18 (m, 2H), 3.90 (d, J = 7.4 Hz, 1H), 3.59 (s, 3H), 3.44-3.31 (m, 1H), 2.58-2.52 (m, 1H), 1.78 (ddt, J = 12.5, 5.2, 2.5 Hz, 1H), 1.62 (ddt, J = 1 3.7, 9.4, 4.8 Hz, 1H), 1.55- 1.47 (m, 1H), 1.45 (s, 9H), 1.38 (td, J = 13.0, 12.3, 3.7 Hz, 1H), 1.06 (d, J = 6.9 Hz, 3H). |
| 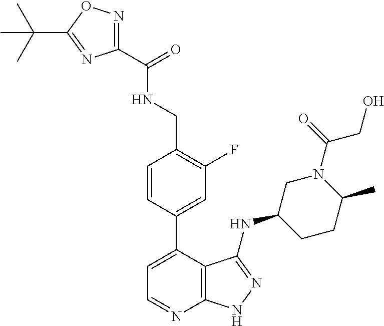 [SMILES: C[C@H]1CC[C@@H](Nc2n[nH]c3nccc(-c4ccc(CNC(=O)c5noc(C(C)(C)C)n5)c(F)c4)c23)CN1C(=O)CO] | 5-(tert-butyl)-N-(2- fluoro-4-(3-(((3R,6S)-1- (2-hydroxyacetyl)-6- methylpiperidin-3- yl)amino)-1H- pyrazolo[3,4-b]pyridin-4- yl)benzyl)-1,2,4- oxadiazole-3- carboxamide | LC-MS (ESI): m/z (M + 1) 565.8 ¹H NMR (400 MHz, DMSO- d₆) δ 12.27 (s, 1H), 9.13 (s, 1H), 8.42 (d, J = 4.7 Hz, 1H), 7.60 (t, J = 7.9 Hz, 1H), 7.44 (dd, J = 5.0, 1.6 Hz, 1H), 7.42 (d, J = 2.0 Hz, 1H), 6.96 (d, J = 4.7 Hz, 1H), 4.64 (d, J = 5.9 Hz, 2H), 4.08 (s, 2H), 3.98 (d, J = 7.2 Hz, 1H), 3.44-3.26 (m, 1H), 2.61 (t, J = 12.0 Hz, 1H), 1.80 (d, J = 11.7 Hz, 1H), 1.63 (dt, J = 14.0, 4.6 Hz, 1H), 1.56-1.49 (m, 2H), 1.45 (s, 9H), 1.11 (d, J = 6.9 Hz, 3H). |
| 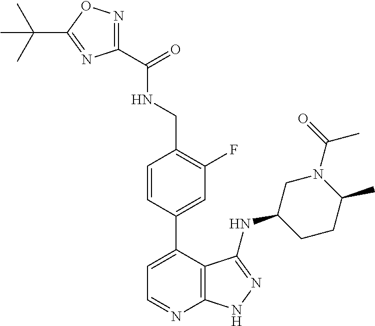 [SMILES: CC(=O)N1C[C@H](Nc2n[nH]c3nccc(-c4ccc(CNC(=O)c5noc(C(C)(C)C)n5)c(F)c4)c23)CC[C@@H]1C] | N-(4-(3-(((3R,6S)-1- acetyl-6-methylpiperidin- 3-yl)amino)-1H- pyrazolo[3,4-b]pyridin-4- yl)-2-fluorobenzyl)-5- (tert-butyl)-1,2,4- oxadiazole-3- carboxamide | LC-MS (ESI): m/z (M + 1) 549.8 ¹H NMR (400 MHz, DMSO- d₆) δ 12.25 (s, 1H), 9.14 (s, 1H), 8.42 (d, J = 4.7 Hz, 1H), 7.60 (t, J = 7.9 Hz, 1H), 7.44 (dd, J = 5.2, 1.6 Hz, 1H), 7.42 (s, 1H), 6.96 (d, J = 4.7 Hz, 1H), 4.64 (d, J = 5.9 Hz, 2H), 4.36 (d, J = 46.1 Hz, 2H), 3.94 (d, J = 7.2 Hz, 1H), 3.34 (q, J = 4.6 Hz, 1H), 2.55 (d, J = 8.3 Hz, 1H), 1.84-1.74 (m, 1H), 1.61 (ddt, J = 13.6, 9.4, 4.8 Hz, 1H), 1.55-1.47 (m, 1H), 1.45 (s, 9H), 1.08 (d, J = 6.9 Hz, 3H). |
| 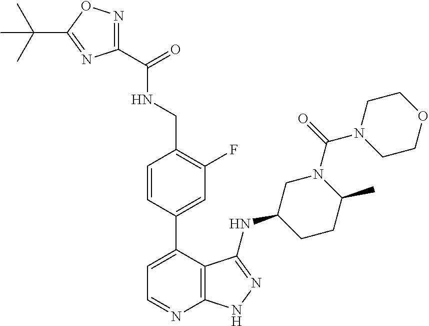 [SMILES: C[C@H]1CC[C@@H](Nc2n[nH]c3nccc(-c4ccc(CNC(=O)c5noc(C(C)(C)C)n5)c(F)c4)c23)CN1C(=O)N1CCOCC1] | 5-(tert-butyl)-N-(2- fluoro-4-(3-(((3R,6S)-6- methyl-1-(morpholine-4- carbonyl)piperidin-3- yl)amino)-1H- pyrazolo[3,4-b]pyridin-4- yl)benzyl)-1,2,4- oxadiazole-3- carboxamide | LC-MS (ESI): m/z (M + 1) 620.9 ¹H NMR (400 MHz, DMSO- d₆) δ 12.24 (s, 1H), 9.13 (s, 1H), 8.42 (d, J = 4.7 Hz, 1H), 7.60 (t, J = 7.9 Hz, 1H), 7.43 (dd, J = 5.3, 1.6 Hz, 1H), 7.41 (d, J = 2.2 Hz, 1H), 6.95 (d, J = 4.7 Hz, 1H), 4.64 (d, J = 5.9 Hz, 2H), 3.92 (d, J = 7.4 Hz, 1H), 3.83 (t, J = 7.2 Hz, 1H), 3.69 (dd, J = 12.7, 4.0 Hz, 1H), 3.52 (t, J = 4.8 Hz, 3H), 3.57- 3.38 (m, 1H), 3.19- 3.04 (m, 3H), 2.68 (dd, J = 13.0, 9.8 Hz, 1H), 1.75 (d, J = 11.5 Hz, 1H), 1.66-1.56 (m, 1H), 1.45 (s, 9H), 1.45 (q, J = 8.4, 7.5 Hz, 4H), 1.07 (d, J = 6.7 Hz, 3H). |
| 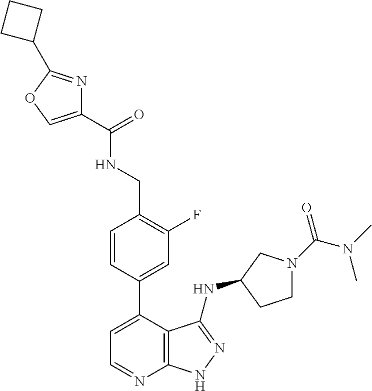 [SMILES: CN(C)C(=O)N1CC[C@@H](Nc2n[nH]c3nccc(-c4ccc(CNC(=O)c5coc(C6CCC6)n5)c(F)c4)c23)C1] | (R)-2-cyclobutyl-N-(4-(3- ((1- (dimethylcarbamoyl) pyrrolidin-3-yl)amino)-1H- pyrazolo[3,4-b]pyridin-4- yl)-2- fluorobenzyl)oxazole-4- carboxamide | LC-MS (ESI): m/z (M + 1) 547.7 ¹H NMR (400 MHz, DMSO- d₆) δ 12.24 (s, 1H), 8.42 (d, J = 4.3 Hz, 2H), 7.55 (t, J = 7.8 Hz, 1H), 7.42-7.36 (m, 3H), 6.95 (d, J = 4.7 Hz, 1H), 4.60 (d, J = 6.1 Hz, 2H), 4.11 (h, J = 5.5 Hz, 1H), 4.05 (d, J = 6.2 Hz, 1H), 3.70 (pd, J = 8.4, 1.1 Hz, 1H), 3.53 (dd, J = 10.7, 5.7 Hz, 1H), 3.34-3.26 (m, 2H), 3.19 (dd, J = 10.8, 4.3 Hz, 1H), 2.72 (s, 6H), 2.44-2.32 (m, 4H), 2.17- 1.88 (m, 3H), 1.71 (dq, J = 12.4, 6.2 Hz, 1H). |
| 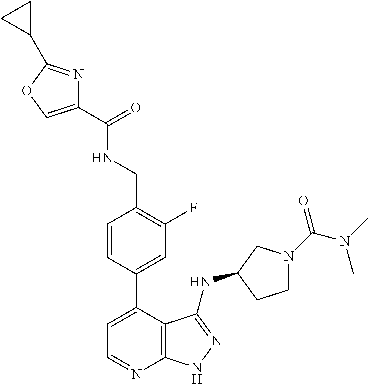 [SMILES: CN(C)C(=O)N1CC[C@@H](Nc2n[nH]c3nccc(-c4ccc(CNC(=O)c5coc(C6CC6)n5)c(F)c4)c23)C1] | (R)-2-cyclopropyl-N-(4- (3-((1- (dimethylcarbamoyl) pyrrolidin-3-yl)amino)-1H- pyrazolo[3,4-b]pyridin-4- yl)-2- fluorobenzyl)oxazole-4- carboxamide | LC-MS (ESI): m/z (M + 1) 533.6 ¹H NMR (400 MHz, DMSO- d₆) δ 12.23 (s, 1H), 8.41 (d, J = 4.7 Hz, 1H), 8.33 (s, 1H), 7.53 (t, J = 7.8 Hz, 1H), 7.42-7.34 (m, 2H), 6.94 (d, J = 4.7 Hz, 1H), 4.58 (d, J = 6.0 Hz, 2H), 4.11 (dt, J = 10.6, 5.4 Hz, 1H), 4.04 (d, J = 6.2 Hz, 1H), 3.54 (dd, J = 10.8, 5.7 Hz, 1H), 3.34- 3.28 (m, 2H), 3.22-3.16 (m, 1H), 2.72 (s, 6H), 2.14 (tt, J = 8.4, 5.0 Hz, 1H), 2.05 (dtd, J = 13.2, 7.5, 5.9 Hz, 1H), 1.71 (tt, J = 12.4, 5.8 Hz, 1H), 1.13-1.04 (m, 2H), 1.04-0.96 (m, 2H). |
| 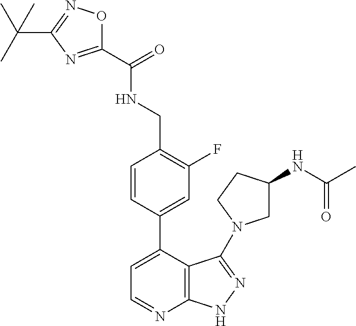 [SMILES: CC(=O)N[C@@H]1CCN(c2n[nH]c3nccc(-c4ccc(CNC(=O)c5nc(C(C)(C)C)no5)c(F)c4)c23)C1] | (R)-N-(4-(3-(3- acetamidopyrrolidin-1- yl)-1H-pyrazolo[3,4- b]pyridin-4-yl)-2- fluorobenzyl)-3-(tert- butyl)-1,2,4-oxadiazole- 5-carboxamide | LC-MS (ESI): m/z (M + 1) 521.6 ¹H NMR (400 MHz, DMSO-d₆) δ 12.51 (s, 1H), 9.51 (s, 1H), 8.42 (d, J = 4.7 Hz, 1H), 7.54 (t, J = 7.7 Hz, 1H), 7.50-7.40 (m, 2H), 7.02 (d, J = 4.6 Hz, 1H), 4.61 (s, 2H), 4.06 (septet, J = 6.8 Hz, 1H), 3.15-3.05 (m, 1H), 2.93-2.83 (m, 1H), 2.77 (ddd, J = 9.6, 7.7, 5.7 Hz, 1H), 2.66 (dd, J = 10.0, 5.9 Hz, 1H), 1.90 (dq, J = 14.5, 7.5 Hz, 1H), 1.74 (s, 3H), 1.56-1.42 (m, 1H), 1.36 (s, 9H). |
| 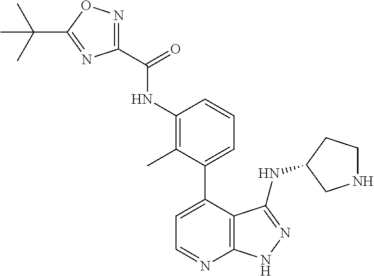 [SMILES: Cc1c(NC(=O)c2noc(C(C)(C)C)n2)cccc1-c1ccnc2[nH]nc(N[C@@H]3CCNC3)c12] | (R)-5-(tert-butyl)-N-(2- methyl-3-(3-(pyrrolidin- 3-ylamino)-1H- pyrazolo[3,4-b]pyridin-4- yl)phenyl)-1,2,4- oxadiazole-3- carboxamide | LC-MS (ESI): m/z (M + 1) 461.1 |
| 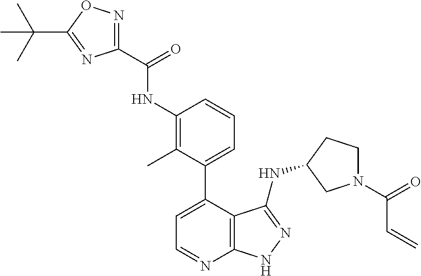 [SMILES: C=CC(=O)N1CC[C@@H](Nc2n[nH]c3nccc(-c4cccc(NC(=O)c5noc(C(C)(C)C)n5)c4C)c23)C1] | (R)-N-(3-(3-((1- acryloylpyrrolidin-3- yl)amino)-1H- pyrazolo[3,4-b]pyridin-4- yl)-2-methylphenyl)-5- (tert-butyl)-1,2,4- oxadiazole-3- carboxamide | LC-MS (ESI): m/z (M + 1) 515.2 |
| 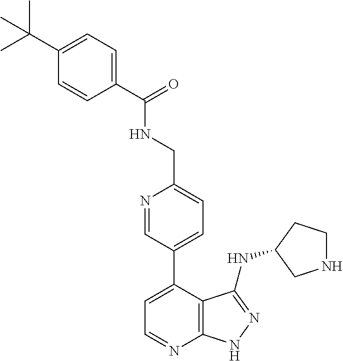 [SMILES: CC(C)(C)c1ccc(C(=O)NCc2ccc(-c3ccnc4[nH]nc(N[C@@H]5CCNC5)c34)cn2)cc1] | (R)-4-(tert-butyl)-N-((5- (3-(pyrrolidin-3- ylamino)-1H- pyrazolo[3,4-b]pyridin-4- yl)pyridin-2- yl)methyl)benzamide | LC-MS (ESI): m/z (M + 1) 470.2 |
| 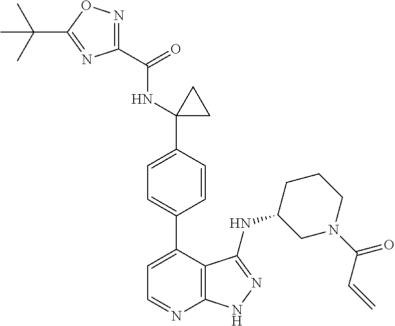 [SMILES: C=CC(=O)N1CCC[C@@H](Nc2n[nH]c3nccc(-c4ccc(C5(NC(=O)c6noc(C(C)(C)C)n6)CC5)cc4)c23)C1] | (R)-N-(1-(4-(3-((1- acryloylpiperidin-3- yl)amino)-1H- pyrazolo[3,4-b]pyridin-4- yl)phenyl)cyclopropyl)-5- (tert-butyl)-1,2,4- oxadiazole-3- carboxamide | LC-MS (ESI): m/z (M + 1) 555.2 ¹H NMR (400 MHz, DMSO-d₆) δ 11.98 (s, 1H), 9.14 (s, 1H), 8.38 (d, J = 4.7 Hz, 1H), 7.52-7.46 (m, 2H), 7.46-7.38 (m, 2H), 6.88 (d, J = 4.7 Hz, 1H), 6.51 (dd, J = 16.8, 10.6 Hz, 1H), 5.94 (dd, J = 16.8, 2.3 Hz, 1H), 5.48 (dd, J = 10.6, 2.3 Hz, 1H), 3.79-3.64 (m, 3H), 3.54-3.35 (m, 3H), 1.87 (dd, J = 12.3, 5.7 Hz, 1H), 1.46 (s, 9H), 1.45-1.34 (m, 5H). |
| 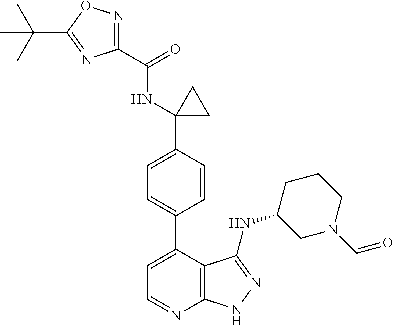 [SMILES: CC(C)(C)c1nc(C(=O)NC2(c3ccc(-c4ccnc5[nH]nc(N[C@@H]6CCCN(C=O)C6)c45)cc3)CC2)no1] | (R)-5-(tert-butyl)-N-(1- (4-(3-((1-frormylpiperidin- 3-yl)amino)-1H- pyrazolo[3,4-b]pyridin-4- yl)phenyl)cyclopropyl)- 1,2,4-oxadiazole-3- carboxamide | LC-MS (ESI): m/z (M + 1) 529.2 |
| 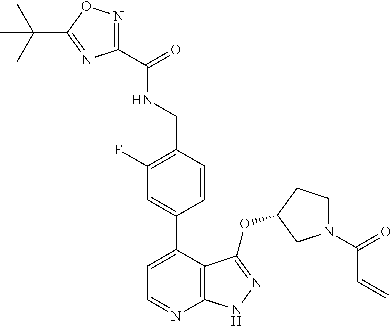 [SMILES: C=CC(=O)N1CC[C@@H](Oc2n[nH]c3nccc(-c4ccc(CNC(=O)c5noc(C(C)(C)C)n5)c(F)c4)c23)C1] | (R)-N-(4-(3-((1- acryloylpyrrolidin-3- yl)oxy)-1H-pyrazolo[3,4- b]pyridin-4-yl)-2- fluorobenzyl)-5-(tert- butyl)-1,2,4-oxadiazole- 3-carboxamide | LC-MS (ESI): m/z (M + 1) 534.2 ¹H NMR (400 MHz, DMSO-d₆) δ 12.45 (s, 1H), 8.89 (s, 1H), 8.50 (d, J = 4.8 Hz, 1H), 7.53-7.43 (m, 3H), 7.13 (d, J = 4.8 Hz, 1H), 6.46 (dd, J = 16.9, 10.5 Hz, 1H), 6.08 (dd, J = 16.9, 2.4 Hz, 1H), 5.59 (dd, J = 10.5, 2.4 Hz, 1H), 5.38 (dq, J = 5.7, 3.0 Hz, 1H), 4.62 (d, J = 5.2 Hz, 2H), 3.71 (s, 2H), 3.61 (t, J = 10.1 Hz, 1H), 3.47-3.35 (m, 1H), 2.27-2.06 (m, 2H), 1.47 (s, 9H). |
| 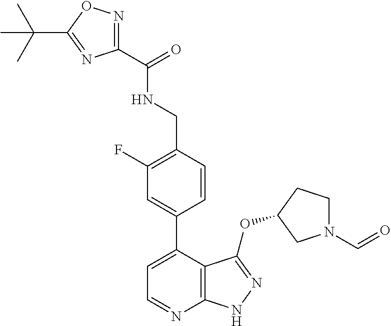 [SMILES: CC(C)(C)c1nc(C(=O)NCc2ccc(-c3ccnc4[nH]nc(O[C@@H]5CCN(C=O)C5)c34)cc2F)no1] | (R)-5-(tert-butyl)-N-(2- fluoro-4-(3-((1- formylpyrrolidin-3- yl)oxy)-1H-pyrazolo[3,4- b]pyridin-4-yl)benzyl)- 1,2,4-oxadiazole-3- carboxamide | LS-MS (ESI): m/z (M + 1) 508.1 ¹H NMR (400 MHz, DMSO-d₆) δ 12.56 (s, 1H), 9.08 (s, 1H), 8.47 (d, J = 4.8 Hz, 1H), 8.09 (d, J = 15.8 Hz, 1H), 7.59-7.39 (m, 2H), 7.12 (d, J = 4.8 Hz, 1H), 5.33 (d, J = 23.2 Hz, 1H), 4.58 (d, J = 5.8 Hz, 2H), 3.69 (d, J = 2.8 Hz, 1H), 3.60-3.48 (m, 2H), 3.44-3.17 (m, 2H), 2.23-1.95 (m, 2H), 1.42 (s, 9H). |
| 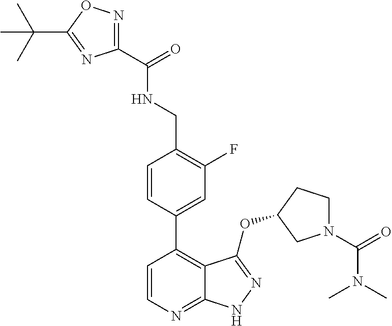 [SMILES: CN(C)C(=O)N1CC[C@@H](Oc2n[nH]c3nccc(-c4ccc(CNC(=O)c5noc(C(C)(C)C)n5)c(F)c4)c23)C1] | (R)-5-(tert-butyl)-N-(4- (3-((1- (dimethylcarbamoyl) pyrrolidin-3-yl)oxy)-1H- pyrazolo[3,4-b]pyridin-4- yl)-2-fluorobenzyl)-1,2,4- oxadiazole-3- carboxamide | LC-MS (ESI): m/z (M + 1) 551.6 ¹H NMR (400 MHz, DMSO-d₆) δ 12.53 (s, 1H), 9.11 (s, 1H), 8.50 (d, J = 4.7 Hz, 1H), 7.53-7.49 (m, 3H), 7.14 (d, J = 4.8 Hz, 1H), 5.32 (tt, J = 4.6, 2.1 Hz, 1H), 4.61 (d, J = 5.9 Hz, 2H), 3.65 (dd, J = 12.1, 4.6 Hz, 1H), 3.48 (dt, J = 12.2, 1.7 Hz, 1H), 3.36 (dd, J = 8.9, 5.2 Hz, 2H), 2.72 (s, 6H), 2.15- 1.96 (m, 2H), 1.45 (s, 9H). |
| 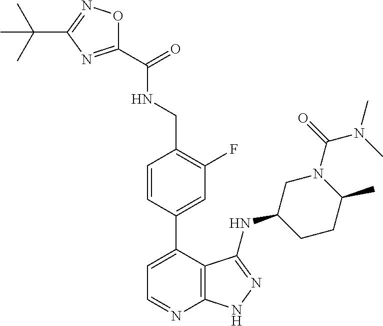 [SMILES: C[C@H]1CC[C@@H](Nc2n[nH]c3nccc(-c4ccc(CNC(=O)c5nc(C(C)(C)C)no5)c(F)c4)c23)CN1C(=O)N(C)C] | 3-(tert-butyl)-N-(4-(3- (((3R,6S)-1- (dimethylcarbamoyl)-6- methylpiperidin-3- yl)amino)-1H- pyrazolo[3,4-b]pyridin-4- yl)-2-fluorobenzyl)-1,2,4- oxadiazole-5- carboxamide | LC-MS (ESI): m/z (M + 1) 578.7 |
| 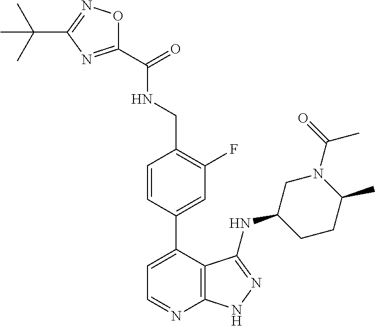 [SMILES: CC(=O)N1C[C@H](Nc2n[nH]c3nccc(-c4ccc(CNC(=O)c5nc(C(C)(C)C)no5)c(F)c4)c23)CC[C@@H]1C] | N-(4-(3-(((3R,6S)-1- acetyl-6-methylpiperidin- 3-yl)amino)-1H- pyrazolo[3,4-b]pyridin-4- yl)-2-fluorobenzyl)-3- (tert-butyl)-1,2,4- oxadiazole-5- carboxamide | LC-MS (ESI): m/z (M + 1) 549.5 |
| 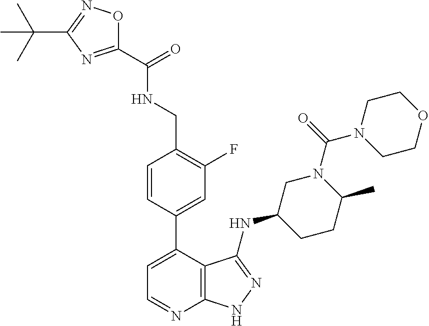 [SMILES: C[C@H]1CC[C@@H](Nc2n[nH]c3nccc(-c4ccc(CNC(=O)c5nc(C(C)(C)C)no5)c(F)c4)c23)CN1C(=O)N1CCOCC1] | 3-(tert-butyl)-N-(2- fluoro-4-(3-(((3R,6S)-6- methyl-1-(morpholine-4- carbonyl)piperidin-3- yl)amino)-1H- pyrazolo[3,4-b]pyridin-4- yl)benzyl)-1,2,4- oxadiazole-5- carboxamide | LC-MS (ESI): m/z (M + 1) 620.9 |
| 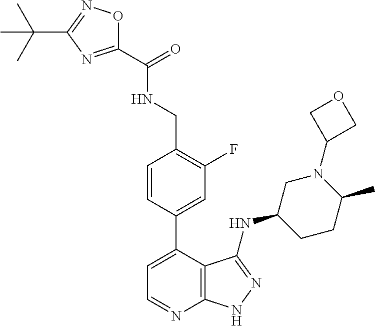 [SMILES: C[C@H]1CC[C@@H](Nc2n[nH]c3nccc(-c4ccc(CNC(=O)c5nc(C(C)(C)C)no5)c(F)c4)c23)CN1C1COC1] | 3-(tert-butyl)-N-(2- fluoro-4-(3-(((3R,6S)-6- methyl-1-(oxetan-3- yl)piperidin-3-yl)amino)- 1H-pyrazolo[3,4- b]pyridin-4-yl)benzyl)- 1,2,4-oxadiazole-5- carboxamide | LC-MS (ESI): m/z (M + 1) 563.5 |
| 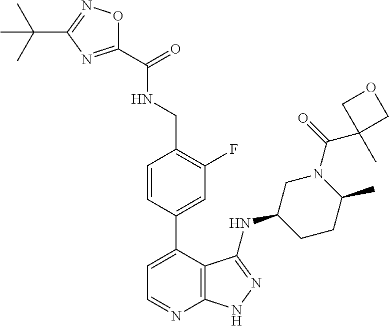 [SMILES: C[C@H]1CC[C@@H](Nc2n[nH]c3nccc(-c4ccc(CNC(=O)c5nc(C(C)(C)C)no5)c(F)c4)c23)CN1C(=O)C1(C)COC1] | 3-(tert-butyl)-N-(2- fluoro-4-(3-(((3R,6S)-6- methyl-1-(3- methyloxetane-3- carbonyl)piperidin-3- yl)amino)-1H- pyrazolo[3,4-b]pyridin-4- yl)benzyl)-1,2,4- oxadiazole-5- carboxamide | LC-MS (ESI): m/z (M + 1) 603.8 |
| 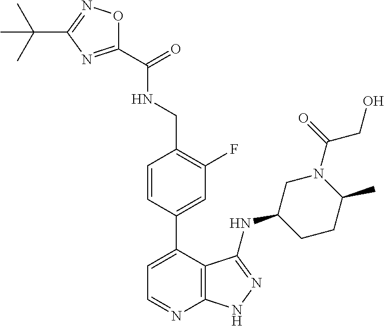 [SMILES: C[C@H]1CC[C@@H](Nc2n[nH]c3nccc(-c4ccc(CNC(=O)c5nc(C(C)(C)C)no5)c(F)c4)c23)CN1C(=O)CO] | 3-(tert-butyl)-N-(2- fluoro-4-(3-(((3R,6S)-1- (2-hydroxyacetyl)-6- methylpiperidin-3- yl)amino)-1H- pyrazolo[3,4-b]pyridin-4- yl)benzyl)-1,2,4- oxadiazole-5- carboxamide | LC-MS (ESI): m/z (M + 1) 565.8 |
| 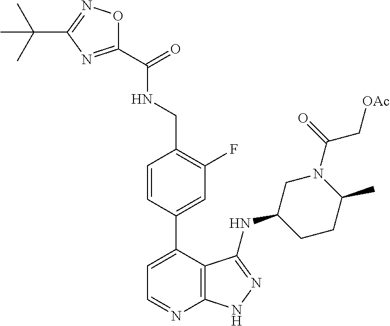 [SMILES: CC(=O)OCC(=O)N1C[C@H](Nc2n[nH]c3nccc(-c4ccc(CNC(=O)c5nc(C(C)(C)C)no5)c(F)c4)c23)CC[C@@H]1C] | 2-((2S,5R)-5-((4-(4-((3- (tert-butyl)-1,2,4- oxadiazole-5- carboxamido)methyl)-3- fluorophenyl)-1H- pyrazolo[3,4-b]pyridin-3- yl)amino)-2- methylpiperidin-1-yl)-2- oxoethyl acetate | LC-MS (ESI): m/z (M + 1) 607.7 |

### Example 56: (R)—N-(4-(3-((1-acryloylpyrrolidin-3-yl)amino)-1H-pyrazolo[3,4-b]pyridin-4-yl)-2-fluorobenzyl)-2-(tert-butyl)oxazole-4-carboxamide

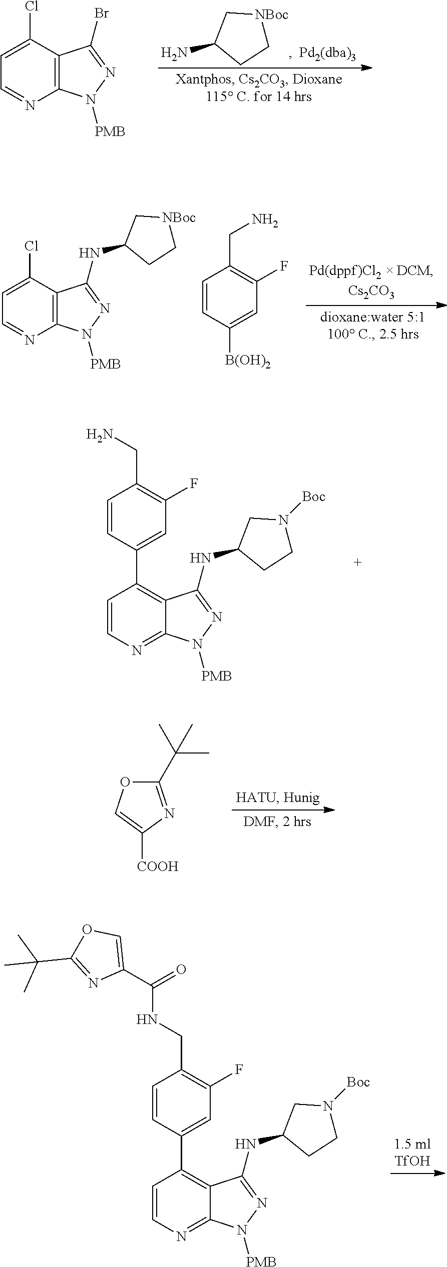

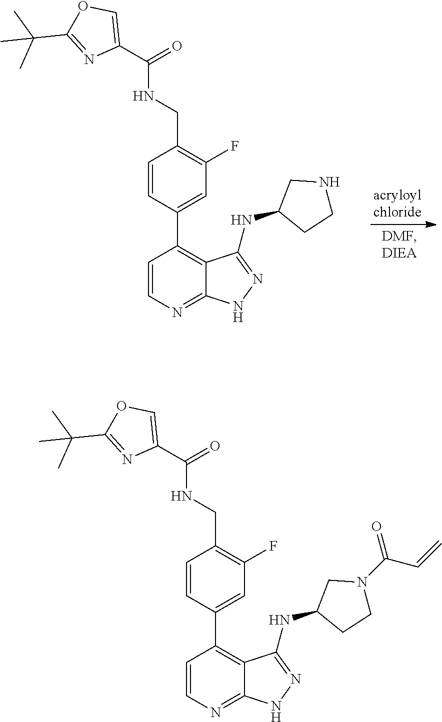 [SMILES: CC(C)(C)c1nc(C(=O)NCc2ccc(-c3ccnc4[nH]nc(N[C@@H]5CCNC5)c34)cc2F)co1 ; C=CC(=O)N1CC[C@@H](Nc2n[nH]c3nccc(-c4ccc(CNC(=O)c5coc(C(C)(C)C)n5)c(F)c4)c23)C1]

To a mixture of: 3-bromo-4-chloro-1-(4-methoxybenzyl)-1H-pyrazolo[3,4-b]pyridine (10 g, 30.7 mmol), tert-butyl (R)-3-aminopyrrolidine-1-carboxylate (7.44 g, 39.9 mmol), Xantphos (2.67 g, 4.61 mmol), and cesium carbonate (15.0 g, 46.1 mmol), in dioxane (90 ml), was added Pd₂(dba)₃ (0.519 g, 0.567 mmol), in a glass bomb and the resulting mixture degassed by bubbling nitrogen through for about 5 minutes. The glass bomb was capped and heated at 115° C. for 14 hrs, then cooled to room temperature. The mixture was diluted with ethyl acetate and water and the layers separated. The aqueous was re-extracted with EtOAc and the combined organics dried over Na₂SO₄, filtered and concentrated. The crude oil was purified by flash chromatography, 0-60% ethyl acetate/hexanes resulting in tert-butyl (R)-3-((4-chloro-1-(4-methoxybenzyl)-1H-pyrazolo[3,4-b]pyridin-3-yl)amino)pyrrolidine-1-carboxylate (7.6 g, 54% yield), as a yellow solid. LC-MS (ESI): m/z (M+1) 458.1.

Into the glass bomb was added: tert-butyl (R)-3-((4-chloro-1-(4-methoxybenzyl)-1H-pyrazolo[3,4-b]pyridin-3-yl)amino)pyrrolidine-1-carboxylate (7.6 g, 16.6 mmol), (4-(aminomethyl)-3-fluorophenyl)boronic acid.HCl (4.42 g, 21.5 mmol), cesium carbonate (10.8 g, 33.1 mmol), and dioxane (60 ml)/water (12 ml). The mixture was degassed by bubbling N2 through for about 10 min, during which time Pd(dppf)Cl₂.DCM (1.35 g, 1.66 mmol) was also added. The glass bomb was then capped and heated at 100° C. for 2.5 hrs. The cooled mixture was worked-up using EtOAc and water:brine mixture. The aqueous was re-extracted with additional EtOAc and the combined organics dried over Na₂SO₄, filtered and concentrated. The crude was purified by normal phase chromatography eluting with 0-20% methanol in dichloromethane, yielding tert-butyl (R)-3-((4-(4-(aminomethyl)-3-fluorophenyl)-1-(4-methoxybenzyl)-1H-pyrazolo[3,4-b]pyridin-3-yl)amino)pyrrolidine-1-carboxylate (8.9 g, 98% yield) as a brown fluffy solid. LC-MS (ESI): m/z (M+1) 547.3.

Into a vial was added: tert-butyl (R)-3-((4-(4-(aminomethyl)-3-fluorophenyl)-1-(4-methoxybenzyl)-1H-pyrazolo[3,4-b]pyridin-3-yl)amino)pyrrolidine-1-carboxylate (200 mg, 0.366 mmol), 2-(tert-butyl)oxazole-4-carboxylic acid (93 mg, 0.549 mmol), HATU (223 mg, 0.585 mmol), DMF (2 ml) and N,N-Diisopropylethylamine (191 μL, 1.10 mmol). The vial was sonicated to dissolve the ingredients and the solution left stirring for 2 hours. The mixture was quenched with water, diluted with NaHCO₃(saturated) and extracted with EtOAc (2×). The combined organics were dried (Na₂SO₄), filtered and concentrated under reduced pressure. The crude liquid was purified by column chromatography on silica gel eluting with 0-100% ethyl acetate in hexanes to give tert-butyl (R)-3-((4-(4-((2-(tert-butyl)oxazole-4-carboxamido)methyl)-3-fluorophenyl)-1-(4-methoxybenzyl)-1H-pyrazolo[3,4-b]pyridin-3-yl)amino)pyrrolidine-1-carboxylate (198 mg, 78% yield as a yellow oily solid. LC-MS (ESI): m/z (M+1) 698.2.

tert-butyl (R)-3-((4-(4-((2-(tert-butyl)oxazole-4-carboxamido)methyl)-3-fluorophenyl)-1-(4-methoxybenzyl)-1H-pyrazolo[3,4-b]pyridin-3-yl)amino)pyrrolidine-1-carboxylate (198 mg, 0.284 mmol) was dissolved in trifluoromethanesulfonic acid (1.5 ml) and stirred 3.5 hrs, then quenched with water and diluted further with DMSO, ACN and water. Upon filtration through a 1μ Acrodisc® PTFE filter, the mixture was purified by reverse-phase HPLC and lyophilized to yield (R)-2-(tert-butyl)-N-(2-fluoro-4-(3-(pyrrolidin-3-ylamino)-1H-pyrazolo[3,4-b]pyridin-4-yl)benzyl)oxazole-4-carboxamide (153 mg) as an orange solid (HCl salt). LC-MS (ESI): m/z (M+1) 478.2.

To a solution of (R)-2-(tert-butyl)-N-(2-fluoro-4-(3-(pyrrolidin-3-ylamino)-1H-pyrazolo[3,4-b]pyridin-4-yl)benzyl)oxazole-4-carboxamide (75 mg, 0.157 mmol) in DMF (1.5 ml) and d N,N-Diisopropylethylamine (82 μL, 0.471 mmol), was added acryloyl chloride (12.8 μL, 0.157) and the resulting solution stirred for 5 minutes, then quenched with water and diluted further with ACN/Water. The crude was purified by reverse-phase HPLC. The isolated fractions were desalted by passing through an Agilent 0.9 mmol PL-HCO₃ MP-Resin cartridge (pre-rinsed with ACN and water) and then lyophilized. The title compound, (R)—N-(4-(3-((1-acryloylpyrrolidin-3-yl)amino)-1H-pyrazolo[3,4-b]pyridin-4-yl)-2-fluorobenzyl)-2-(tert-butyl)oxazole-4-carboxamide (45 mg, 54% yield) was obtained as a pale yellow solid. LC-MS (ESI): m/z (M+1) 532.2.

The following compounds were prepared by analogous methods:

| Chemical Structure | Chemical Name | LC-MS, ¹H NMR |
|---|---|---|
| 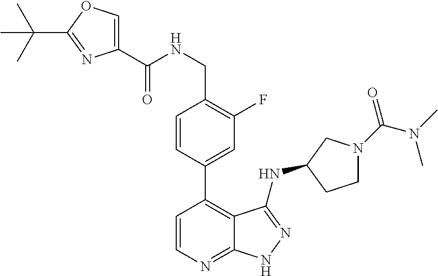 [SMILES: CN(C)C(=O)N1CC[C@@H](Nc2n[nH]c3nccc(-c4ccc(CNC(=O)c5coc(C(C)(C)C)n5)c(F)c4)c23)C1] | (R)-2-(tert-butyl)-N-(4- (3-((1- (dimethylcarbamoyl) pyrrolidin-3-yl)amino)-1H- pyrazolo[3,4-b]pyridin-4- yl)-2- fluorobenzyl)oxazole-4- carboxamide | LC-MS (ESI): m/z (M + 1) 549.3. |
| 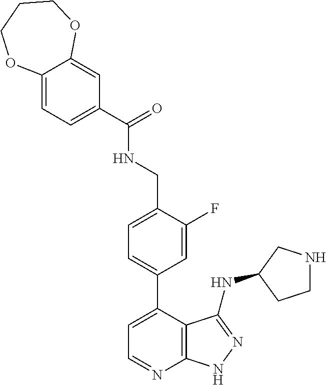 [SMILES: O=C(NCc1ccc(-c2ccnc3[nH]nc(N[C@@H]4CCNC4)c23)cc1F)c1ccc2c(c1)OCCCO2] | (R)-N-(2-fluoro-4-(3- (pyrrolidin-3-ylamino)- 1H-pyrazolo[3,4- b]pyridin-4-yl)benzyl)- 3,4-dihydro-2H- benzo[b][1,4]dioxepine- 7-carboxamide | LC-MS: m/z (M + 1) 503.4. |
| 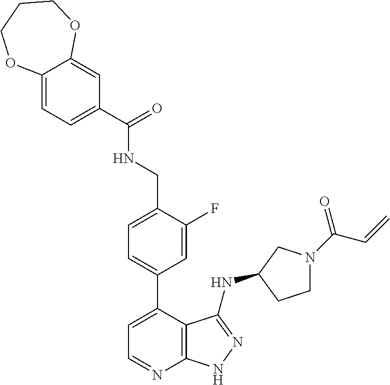 [SMILES: C=CC(=O)N1CC[C@@H](Nc2n[nH]c3nccc(-c4ccc(CNC(=O)c5ccc6c(c5)OCCCO6)c(F)c4)c23)C1] | (R)-N-(4-(3-((1- acryloylpyrrolidin-3- yl)amino)-1H- pyrazolo[3,4-b]pyridin-4- yl)-2-fluorobenzyl)-3,4- dihydro-2H- benzo[b][1,4]dioxepine- 7-carboxamide | LC-MS: m/z (M + 1) 557.4. |
| 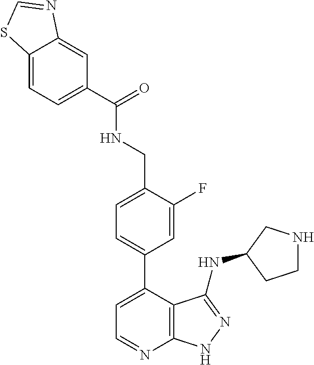 [SMILES: O=C(NCc1ccc(-c2ccnc3[nH]nc(N[C@@H]4CCNC4)c23)cc1F)c1ccc2scnc2c1] | (R)-N-(2-fluoro-4-(3- (pyrrolidin-3-ylamino)- 1H-pyrazolo[3,4- b]pyridin-4- yl)benzyl)benzo[d]thiazole- 5-carboxamide | LC-MS: m/z (M + 1) 488.3. |
| 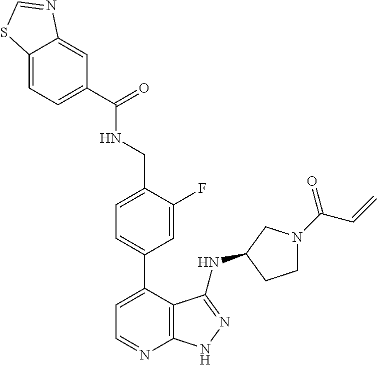 [SMILES: C=CC(=O)N1CC[C@@H](Nc2n[nH]c3nccc(-c4ccc(CNC(=O)c5ccc6scnc6c5)c(F)c4)c23)C1] | (R)-N-(4-(3-((1- acryloylpyrrolidin-3- yl)amino)-1H- pyrazolo[3,4-b]pyridin-4- yl)-2- fluorobenzyl)benzo[d]thia- zole-5-carboxamide | LC-MS: m/z (M + 1) 542.2. |
| 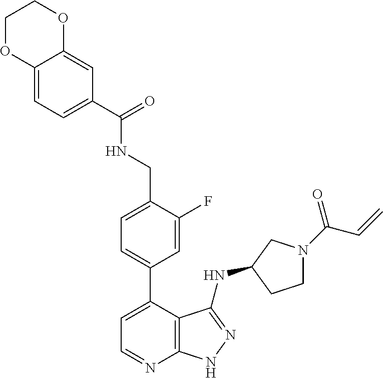 [SMILES: C=CC(=O)N1CC[C@@H](Nc2n[nH]c3nccc(-c4ccc(CNC(=O)c5ccc6c(c5)OCCO6)c(F)c4)c23)C1] | (R)-N-(4-(3-((1- acryloylpyrrolidin-3- yl)amino)-1H- pyrazolo[3,4-b]pyridin-4- yl)-2-fluorobenzyl)-2,3- dihydrobenzo[b][1,4]diox- ine-6-carboxamide | LC-MS: m/z (M + 1) 543.4. |
| 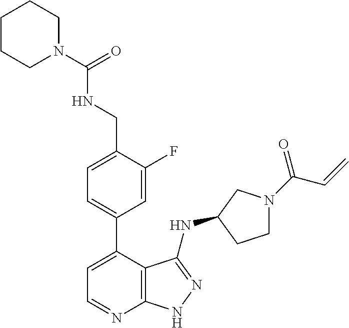 [SMILES: C=CC(=O)N1CC[C@@H](Nc2n[nH]c3nccc(-c4ccc(CNC(=O)N5CCCCC5)c(F)c4)c23)C1] | (R)-N-(4-(3-((1- acryloylpyrrolidin-3- yl)amino)-1H- pyrazolo[3,4-b]pyridin-4- yl)-2- fluorobenzyl)piperidine- 1-carboxamide | LC-MS: m/z (M + 1) 492.5. |
| 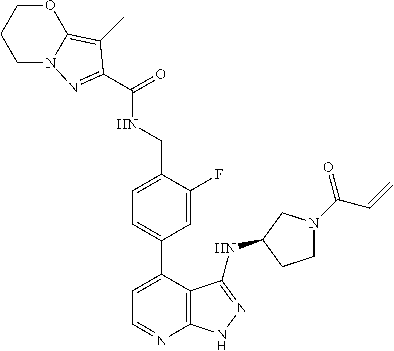 [SMILES: C=CC(=O)N1CC[C@@H](Nc2n[nH]c3nccc(-c4ccc(CNC(=O)c5nn6c(c5C)OCCC6)c(F)c4)c23)C1] | (R)-N-(4-(3-((1- acryloylpyrrolidin-3- yl)amino)-1H- pyrazolo[3,4-b]pyridin-4- yl)-2-fluorobenzyl)-3- methyl-6,7-dihydro-5H- pyrazolo[5,1- b][1,3]oxazine-2- carboxamide | LC-MS: m/z (M + 1) 545.4. |
| 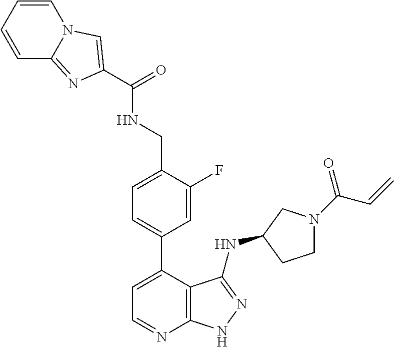 [SMILES: C=CC(=O)N1CC[C@@H](Nc2n[nH]c3nccc(-c4ccc(CNC(=O)c5cn6ccccc6n5)c(F)c4)c23)C1] | (R)-N-(4-(3-((1- acryloylpyrrolidin-3- yl)amino)-1H- pyrazolo[3,4-b]pyridin-4- yl)-2- fluorobenzyl)imidazo[1,2- a]pyridine-2- carboxamide | LC-MS: m/z (M + 1) 525.4. |
| 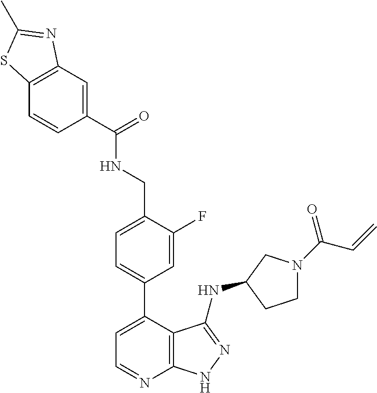 [SMILES: C=CC(=O)N1CC[C@@H](Nc2n[nH]c3nccc(-c4ccc(CNC(=O)c5ccc6sc(C)nc6c5)c(F)c4)c23)C1] | (R)-N-(4-(3-((1- acryloylpyrrolidin-3- yl)amino)-1H- pyrazolo[3,4-b]pyridin-4- yl)-2-fluorobenzyl)-2- methylbenzo[d]thiazole- 5-carboxamide | LC-MS: m/z (M + 1) 556.5. |
| 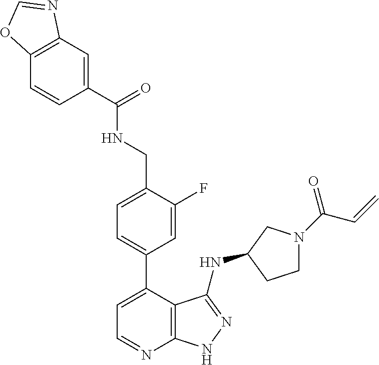 [SMILES: C=CC(=O)N1CC[C@@H](Nc2n[nH]c3nccc(-c4ccc(CNC(=O)c5ccc6ocnc6c5)c(F)c4)c23)C1] | (R)-N-(4-(3-((1- acryloylpyrrolidin-3- yl)amino)-1H- pyrazolo[3,4-b]pyridin-4- yl)-2- fluorobenzyl)benzo[d]oxa- zole-5-carboxamide | LC-MS: m/z (M + 1) 526.4. |
| 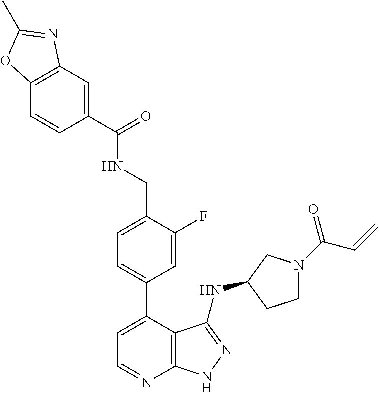 [SMILES: C=CC(=O)N1CC[C@@H](Nc2n[nH]c3nccc(-c4ccc(CNC(=O)c5ccc6oc(C)nc6c5)c(F)c4)c23)C1] | (R)-N-(4-(3-((1- acryloylpyrrolidin-3- yl)amino)-1H- pyrazolo[3,4-b]pyridin-4- yl)-2-fluorobenzyl)-2- methylbenzo[d]oxazole- 5-carboxamide | LC-MS: m/z (M + 1) 540.5. |
| 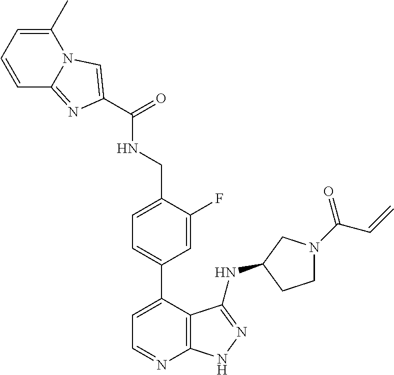 [SMILES: C=CC(=O)N1CC[C@@H](Nc2n[nH]c3nccc(-c4ccc(CNC(=O)c5cn6c(C)cccc6n5)c(F)c4)c23)C1] | (R)-N-(4-(3-((1- acryloylpyrrolidin-3- yl)amino)-1H- pyrazolo[3,4-b]pyridin-4- yl)-2-fluorobenzyl)-5- methylimidazo[1,2- a]pyridine-2-carboxamide | LC-MS: m/z (M + 1) 539.4. |

### Example 57: 3-(tert-butyl)-N-(2-fluoro-4-(3-(4-methyl-2,3-dioxopiperazin-1-yl)-1H-pyrazolo[3,4-b]pyridin-4-yl)benzyl)-1,2,4-oxadiazole-5-carboxamide

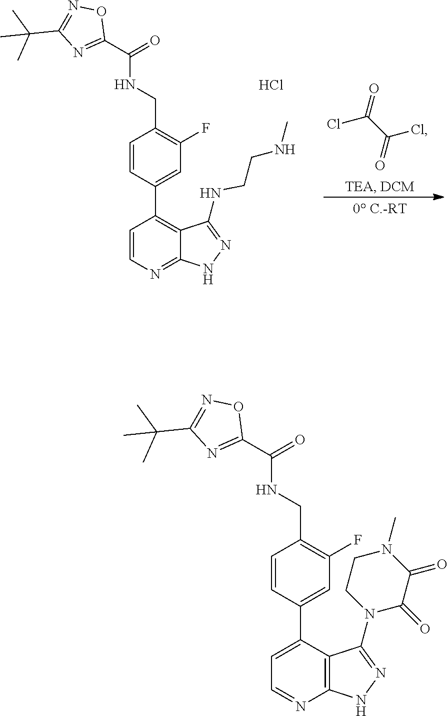

A solution of 3-(tert-butyl)-N-(2-fluoro-4-(3-((2-(methylamino)ethyl)amino)-1H-pyrazolo[3,4-b]pyridin-4-yl)benzyl)-1,2,4-oxadiazole-5-carboxamide hydrochloride (45 mg, 0.090 mmol) in DCM (1 ml) and triethylamine (38 μL, 0.270 mmol), was cooled in an ice bath. Oxalyl chloride (15 μL, 0.180 mmol) was added and the resulting solution stirred at 0° C. for 5 min, then the ice bath was removed and the solution stirred another 20 min at room temperature. The reaction was quenched with NaHCO₃(saturated), diluted with water and extracted with DCM (3×). The combined organics were dried (Na₂SO₄), filtered and concentrated under reduced pressure. The crude yellow solid was purified by RP-HPLC and the isolated fractions desalted by passing through an Agilent 0.9 mmol PL-HCO₃ MP-Resin cartridge (pre-rinsed with ACN and water). Upon lyophilization, 3-(tert-butyl)-N-(2-fluoro-4-(3-(4-methyl-2,3-dioxopiperazin-1-yl)-1H-pyrazolo[3,4-b]pyridin-4-yl)benzyl)-1,2,4-oxadiazole-5-carboxamide (12 mg, 15% yield) was obtained, as a white solid. LC-MS (ESI): m/z (M+1) 521.6.

### Example 58: (R)—N-(4-(3-(3-(3,3-dimethylureido)pyrrolidin-1-yl)-1H-pyrazolo[3,4-b]pyridin-4-yl)-2-fluorobenzyl)-2-fluoro-4-(2-hydroxypropan-2-yl)benzamide

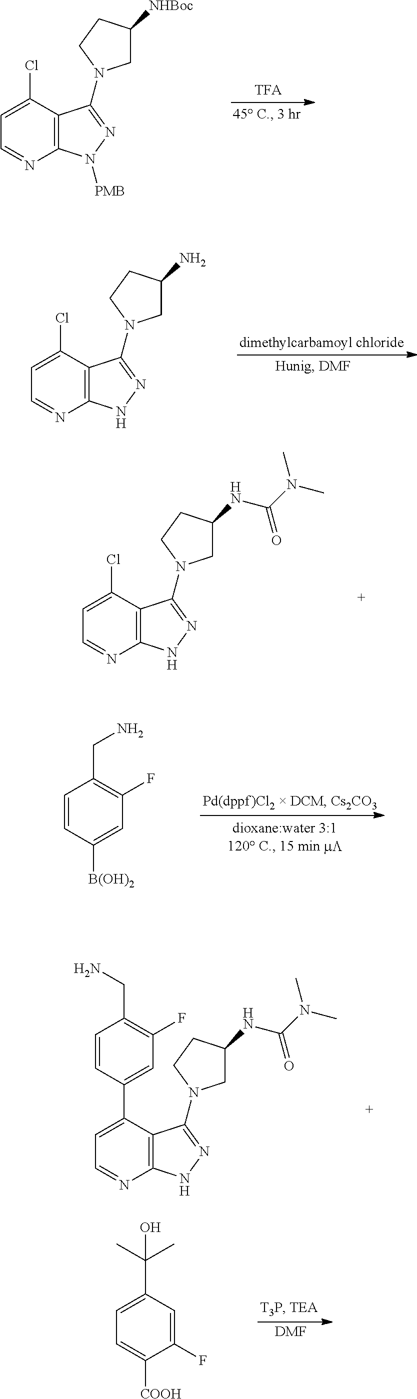

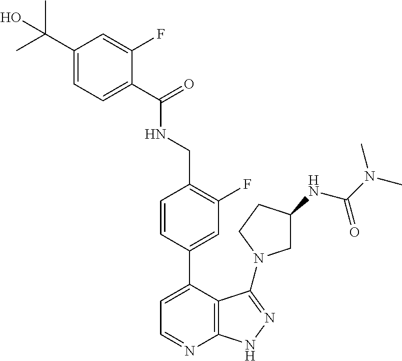 [SMILES: CN(C)C(=O)N[C@@H]1CCN(c2n[nH]c3nccc(-c4ccc(CNC(=O)c5ccc(C(C)(C)O)cc5F)c(F)c4)c23)C1]

(R)-tert-butyl (1-(4-chloro-1-(4-methoxybenzyl)-1H-pyrazolo[3,4-b]pyridin-3-yl)pyrrolidin-3-yl)carbamate (1.0 g, 2.18 mmol) was dissolved in 2,2,2-trifluoroacetic acid (8 ml, 104 mmol) and stirred at room temperature for 5 min, then heated at 450 for 3 hrs. Upon cooling to room temperature, the reaction was diluted with DCM, and the volatiles removed in vacuo. The residue was re-dissolved in DCM and basidified by addition of 1 ml TEA, then concentrated again. The crude was purified by flash chromatography, 0-50% MeOH/0.1% TEA in DCM, yielding (R)-1-(4-chloro-1H-pyrazolo[3,4-b]pyridin-3-yl)pyrrolidin-3-amine (452 mg, 87% yield) as a yellow oily solid. The product appears as 1-2 peaks: MH⁺=238.0/239.9 @0.25 min/1.0 min and MH⁺=238.1/239.9 @ 0.38 min/1.0 min LCMS.

To a slurry of (R)-1-(4-chloro-1H-pyrazolo[3,4-b]pyridin-3-yl)pyrrolidin-3-amine (226 mg, 0.951 mmol) in DMF (5 ml) and Hunig's base (0.498 ml, 2.85 mmol), was added dimethylcarbamoyl chloride (0.437 ml, 4.75 mmol). After about 10 minutes of stirring, another portion of carbamoyl chloride (250 μl, 2.72 mmol) was added and the mixture stirred another 10 min, then quenched with dilute NaHCO₃(sat.), and extracted with EtOAc. Aqueous layer was re-extracted 2× with ethyl acetate and 2× with DCM. The combined organics were dried over Na₂SO₄, filtered and concentrated in vacuo. The crude yellow liquid was purified by flash chromatography, eluting with 0-30% MeOH/DCM. The product, (R)-3-(1-(4-chloro-1H-pyrazolo[3,4-b]pyridin-3-yl)pyrrolidin-3-yl)-1,1-dimethylurea (285 mg, 97% yield) was isolated as a yellow oil. MH⁺=309.3/311.0 @ 0.57 min/1.0 min.

In a 5 ml microwave reaction vial, (4-(aminomethyl)-3-fluorophenyl)boronic acid.HCl (379 mg, 1.845 mmol), cesium carbonate (902 mg, 2.77 mmol), Pd(dppf)Cl₂.DCM (75 mg, 0.092 mmol), and (R)-3-(1-(4-chloro-1H-pyrazolo[3,4-b]pyridin-3-yl)pyrrolidin-3-yl)-1,1-dimethylurea (285 mg, 0.923 mmol) in dioxane (3741 μl) and water (1247 μl) were added to give an orange suspension. The suspension was heated in a Biotage® microwave at 120° C. for 15 min, upon which time more of the boronic acid (190 mg, 0.925 mmol) and catalyst (35 mg, 0.043 mmol) were added. The mixture was re-submitted for another 15 min at 120° C. and upon cooling parted between EtOAc and water. The aqueous was re-extracted 1× with EtOAc, 3-4× with DCM, and the combined organics dried (Na₂SO₄), filtered and concentrated. The crude was purified by column chromatography on silica gel eluting with 0-50% MeOH in DCM, resulting in (R)-3-(1-(4-(4-(aminomethyl)-3-fluorophenyl)-1H-pyrazolo[3,4-b]pyridin-3-yl)pyrrolidin-3-yl)-1,1-dimethylurea (94 mg, 26% yield) as a light brown oily solid. MH⁺=398.3 @0.42 min/1.0 min.

To a solution of (R)-3-(1-(4-(4-(aminomethyl)-3-fluorophenyl)-1H-pyrazolo[3,4-b]pyridin-3-yl)pyrrolidin-3-yl)-1,1-dimethylurea (43.8 mg, 0.110 mmol) and 2-fluoro-4-(2-hydroxypropan-2-yl)benzoic acid (32.8 mg, 0.165 mmol) in DMF (1 ml) and Hunig's base (0.096 ml, 0.551 mmol), was added 2,4,6-tripropyl-1,3,5,2,4,6-trioxatriphosphinane 2,4,6-trioxide (50% ethyl acetate solution) (0.130 ml, 0.220 mmol) and the brown solution stirred for 30 min, then quenched with water, diluted further with ACN and water and purified directly by RP HPLC. The isolated product fractions were desalted by passing through an Agilent 0.9 mmol PL-HCO₃ MP-Resin cartridge (pre-rinsed with ACN and water) and then lyophilized to yield (R)—N-(4-(3-(3-(3,3-dimethylureido)pyrrolidin-1-yl)-1H-pyrazolo[3,4-b]pyridin-4-yl)-2-fluorobenzyl)-2-fluoro-4-(2-hydroxypropan-2-yl)benzamide (15.4 mg, 23% yield). LC-MS (ESI): m/z (M+1) 578.8.

The following compounds were prepared by analogous methods:

| Chemical Structure | Chemical Name | LC-MS, ¹H NMR |
|---|---|---|
| 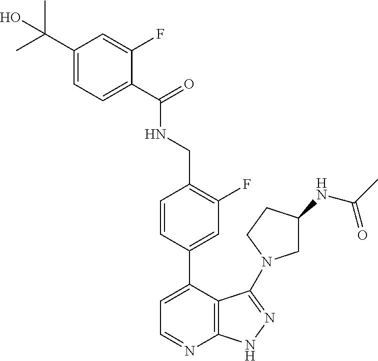 [SMILES: CC(=O)N[C@@H]1CCN(c2n[nH]c3nccc(-c4ccc(CNC(=O)c5ccc(C(C)(C)O)cc5F)c(F)c4)c23)C1] | (R)-N-(4-(3-(3- acetamidopyrrolidin-1- yl)-1H-pyrazolo[3,4- b]pyridin-4-yl)-2- fluorobenzyl)-2-fluoro-4- (2-hydroxypropan-2- yl)benzamide | LC-MS (ESI): m/z (M + 1) 549.8. |
| 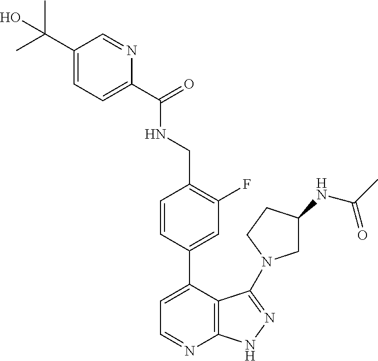 [SMILES: CC(=O)N[C@@H]1CCN(c2n[nH]c3nccc(-c4ccc(CNC(=O)c5ccc(C(C)(C)O)cn5)c(F)c4)c23)C1] | (R)-N-(4-(3-(3- acetamidopyrrolidin-1- yl)-1H-pyrazolo[3,4- b]pyridin-4-yl)-2- fluorobenzyl)-5-(2- hydroxypropan-2- yl)picolinamide | LC-MS (ESI): m/z (M + 1) 532.6. |
| 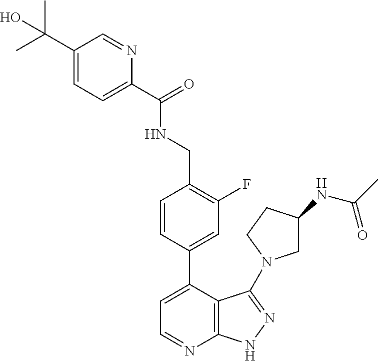 | (R)-N-(4-(3-(3-(3,3- dimethylureido)pyrrolidin- 1-yl)-1H-pyrazolo[3,4- b]pyridin-4-yl)-2- fluorobenzyl)-5-(2- hydroxypropan-2- yl)picolinamide | LC-MS (ESI): m/z (M + 1) 561.6. |
| 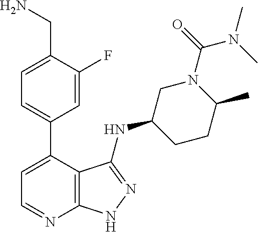 [SMILES: C[C@H]1CC[C@@H](Nc2n[nH]c3nccc(-c4ccc(CN)c(F)c4)c23)CN1C(=O)N(C)C] | (2S,5R)-5-((4-(4- (aminomethyl)-3- fluorophenyl)-1H- pyrazolo[3,4-b]pyridin-3- yl)amino)-N,N,2- trimethylpiperidine-1- carboxamide | LC-MS: m/z (M + 1) 426.6. |
| 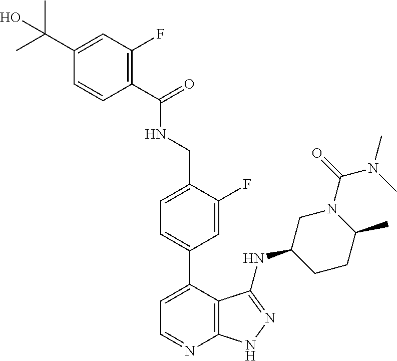 [SMILES: C[C@H]1CC[C@@H](Nc2n[nH]c3nccc(-c4ccc(CNC(=O)c5ccc(C(C)(C)O)cc5F)c(F)c4)c23)CN1C(=O)N(C)C] | (2S,5R)-5-((4-(3-fluoro- 4-((2-fluoro-4-(2- hydroxypropan-2- yl)benzamido)methyl)phen- yl)-1H-pyrazolo[3,4- b]pyridin-3-yl)amino)- N,N,2- trimethylpiperidine-1- carboxamide | LC-MS: m/z (M + 1) 606.9. |
| 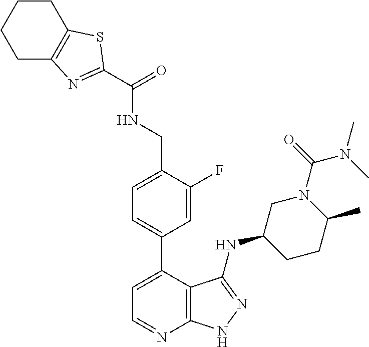 [SMILES: C[C@H]1CC[C@@H](Nc2n[nH]c3nccc(-c4ccc(CNC(=O)c5nc6c(s5)CCCC6)c(F)c4)c23)CN1C(=O)N(C)C] | N-(4-(3-(((3R,6S)-1- (dimethylcarbamoyl)-6- methylpiperidin-3- yl)amino)-1H- pyrazolo[3,4-b]pyridin-4- yl)-2-fluorobenzyl)- 4,5,6,7- tetrahydrobenzo[d]thiazole- 2-carboxamide | LC-MS: m/z (M + 1) 591.9. |
| 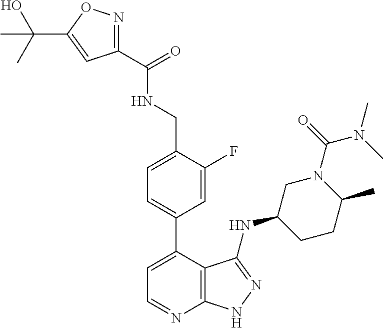 [SMILES: C[C@H]1CC[C@@H](Nc2n[nH]c3nccc(-c4ccc(CNC(=O)c5cc(C(C)(C)O)on5)c(F)c4)c23)CN1C(=O)N(C)C] | N-(4-(3-(((3R,6S)-1- (dimethylcarbamoyl)-6- methylpiperidin-3- yl)amino)-1H- pyrazolo[3,4-b]pyridin-4- yl)-2-fluorobenzyl)-5-(2- hydroxypropan-2- yl)isoxazole-3- carboxamide | LC-MS: m/z (M + 1) 579.7. |
| 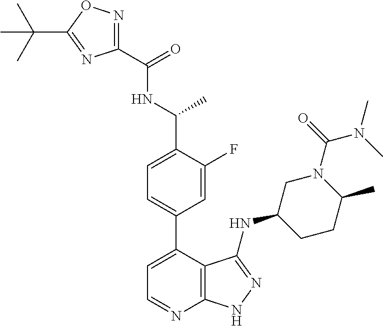 [SMILES: C[C@@H](NC(=O)c1noc(C(C)(C)C)n1)c1ccc(-c2ccnc3[nH]nc(N[C@@H]4CC[C@H](C)N(C(=O)N(C)C)C4)c23)cc1F] | 5-(tert-butyl)-N-((R)-1- (4-(3-(((3R,6S)-1- (dimethylcarbamoyl)-6- methylpiperidin-3- yl)amino)-1H- pyraozlo[3,4-b]pyridin-4- yl)-2-fluorophenyl)ethyl)- 1,2,4-oxadiazole-3- carboxamide | LC-MS: m/z (M + 1) 592.6. |
| 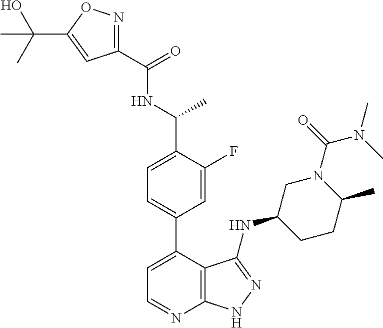 [SMILES: C[C@@H](NC(=O)c1cc(C(C)(C)O)on1)c1ccc(-c2ccnc3[nH]nc(N[C@@H]4CC[C@H](C)N(C(=O)N(C)C)C4)c23)cc1F] | N-((R)-1-(4-(3-(((3R,6S)- 1-(dimethylcarbamoyl)-6- methylpiperidin-3- yl)amino)-1H- pyrazolo[3,4-b]pyridin-4- yl)-2-fluorophenyl)ethyl)- 5-(2-hydroxypropan-2- yl)isoxazole-3- carboxamide | LC-MS: m/z (M + 1) 593.6. |
| 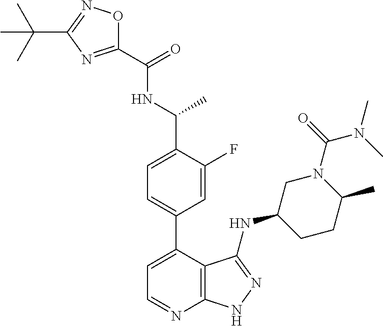 [SMILES: C[C@@H](NC(=O)c1nc(C(C)(C)C)no1)c1ccc(-c2ccnc3[nH]nc(N[C@@H]4CC[C@H](C)N(C(=O)N(C)C)C4)c23)cc1F] | 3-(tert-butyl)-N-((R)-1- (4-(3-(((3R,6S)-1- (dimethylcarbamoyl)-6- methylpiperidin-3- yl)amino)-1H- pyraozlo[3,4-b]pyridin-4- yl)-2-fluorophenyl)ethyl)- 1,2,4-oxadiazole-5- carboxamide | LC-MS: m/z (M + 1) 592.9. |
| 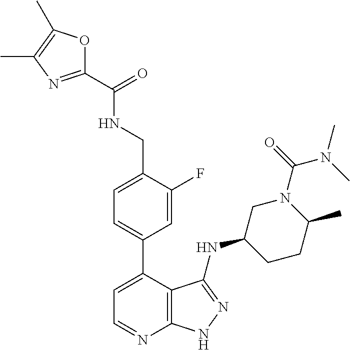 | N-(4-(3-(((3R,6S)-1- (dimethylcarbamoyl)-6- methylpiperidin-3- yl)amino)-1H- pyrazolo[3,4-b]pyridin-4- yl)-2-fluorobenzyl)-4,5- dimethyloxazole-2- carboxamide | LC-MS: m/z (M + 1) 549.6. |

### Example 59: (R)-3-((4-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-1H-pyrazolo[3,4-b]pyridin-3-yl)amino)-N,N-dimethylpyrrolidine-1-carboxamide

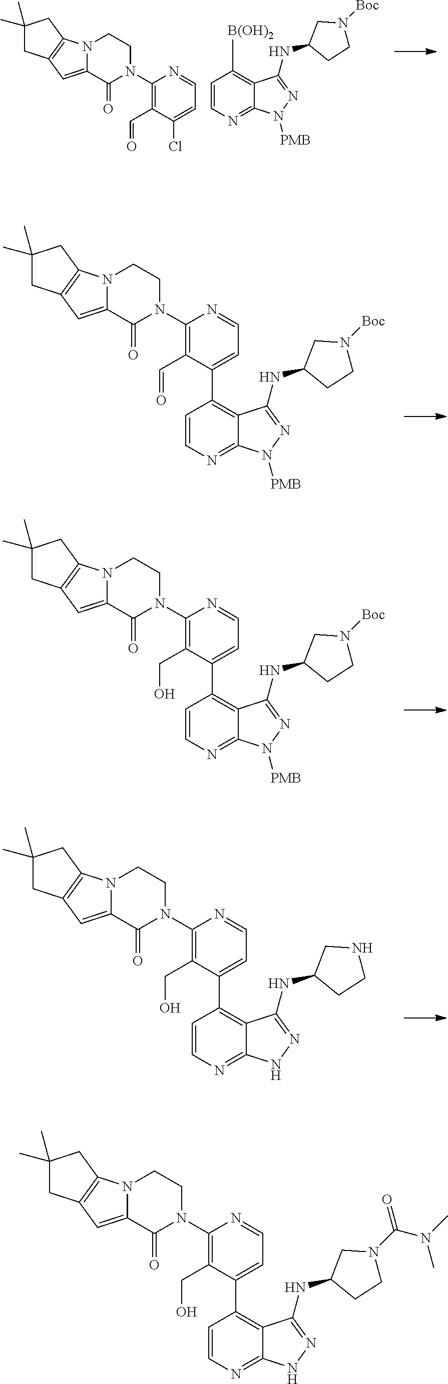

The mixture of 4-chloro-2-(7,7-dimethyl-1-oxo-1,3,4,6,7,8-hexahydro-2H-cyclopenta[4,5]pyrrolo[1,2-a]pyrazin-2-yl)nicotinaldehyde (CAS: 1434050-55-1) (100 mg, 0.29 mmol), (R)-(3-((1-(tert-butoxycarbonyl)pyrrolidin-3-yl)amino)-1-(4-methoxybenzyl)-1H-pyrazolo[3,4-b]pyridin-4-yl)boronic acid (270 mg, 0.58 mmol), Pd(dppf)Cl2 (82 mg, 0.1 mmol), cesium carbonate (750 mg, 2.3 mmol) in 30 mL dioxane and 5 mL water was degassed using nitrogen stream for 5 min. It was then sent to 115° C. to be stirred in nitrogen atmosphere for overnight. It was cooled to room temperature, concentrated in vacuo, diluted with 100 mL ethyl acetate, washed with water, concentrated, and subjected to flash column with 50-100% ethyl acetate in DCM to isolate tert-butyl (R)-3-((4-(2-(7,7-dimethyl-1-oxo-1,3,4,6,7,8-hexahydro-2H-cyclopenta[4,5]pyrrolo[1,2-a]pyrazin-2-yl)-3-formylpyridin-4-yl)-1-(4-methoxybenzyl)-1H-pyrazolo[3,4-b]pyridin-3-yl)amino)pyrrolidine-1-carboxylate. It was dissolved in 15 mL methanol and stirred in ice bath. To it was added NaBH₄ (33 mg, 0.87 mmol), and then more (33 mg, 0.87 mmol) in 90 min. The mixture was stirred for 30 min, diluted with water, concentrated and directly subjected to flash column using 0-10% methanol to isolate tert-butyl (R)-3-((4-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-1-(4-methoxybenzyl)-1H-pyrazolo[3,4-b]pyridin-3-yl)amino)pyrrolidine-1-carboxylate. It was treated with 6 mL TFA and 1 mL TfOH at room temperature for 2.5 hours. It was diluted with 2 mL DMF and 2 mL water, and directly subjected to reverse phase preparative HPLC to isolate (R)-2-(3-(hydroxymethyl)-4-(3-(pyrrolidin-3-ylamino)-1H-pyrazolo[3,4-b]pyridin-4-yl)pyridin-2-yl)-7,7-dimethyl-3,4,7,8-tetrahydro-2H-cyclopenta[4,5]pyrrolo[1,2-a]pyrazin-1(6H)-one (41 mg) as HCl salt. LC-MS (ESI): m/z (M+1) 513.2, (M−1) 511.3.

(R)-2-(3-(Hydroxymethyl)-4-(3-(pyrrolidin-3-ylamino)-1H-pyrazolo[3,4-b]pyridin-4-yl)pyridin-2-yl)-7,7-dimethyl-3,4,7,8-tetrahydro-2H-cyclopenta[4,5]pyrrolo[1,2-a]pyrazin-1(6H)-one HCl salt (13 mg, 0.022 mmol) was dissolved in 4 mL DMF. To it were added DIPEA (38 μL, 0.22 mmol) and then dimethylcarbamic chloride (10 μL, 0.11 mmol). The reaction was cleanly complete in 10 min. The reaction mixture was quenched with TFA (100 μL) and directly subjected to reverse phase preparative HPLC to isolate (R)-3-((4-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-1H-pyrazolo[3,4-b]pyridin-3-yl)amino)-N,N-dimethylpyrrolidine-1-carboxamide as HCl salt (7.1 mg). LC-MS (ESI): m/z (M+1) 584.3, (M−1) 582.3.

The following compounds were made using the same synthetic scheme:

| Chemical Structure | Chemical Name | LC-MS, ¹H NMR |
|---|---|---|
| 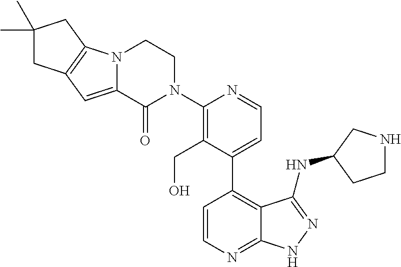 [SMILES: CC1(C)Cc2cc3n(c2C1)CCN(c1nccc(-c2ccnc4[nH]nc(N[C@@H]5CCNC5)c24)c1CO)C3=O] | (R)-2-(3- (hydroxymethyl)-4-(3- (pyrrolidin-3-ylamino)- 1H-pyrazolo[3,4- b]pyridin-4-yl)pyridin-2- yl)-7,7-dimethyl-3,4,7,8- tetrahydro-2H- cyclopentan[4,5]pyrrolo [1,2-a]pyrazin-1(6H)- one | LC-MS (ESI): m/z (M + 1) 513.2, (M − 1) 511.3. |
| 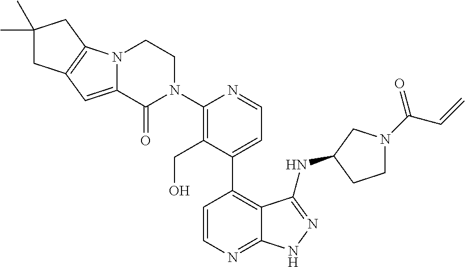 [SMILES: C=CC(=O)N1CC[C@@H](Nc2n[nH]c3nccc(-c4ccnc(N5CCn6c(cc7c6CC(C)(C)C7)C5=O)c4CO)c23)C1] | (R)-2-(4-(3-((1- acryloylpyrrolidin-3- yl)amino)-1H- pyrazolo[3,4-b]pyridin- 4-yl)-3- (hydroxymethyl)pyridin- 2-yl)-7,7-dimethyl- 3,4,7,8-tetrahydro-2H- cyclopenta[4.5]pyrrolo [1,2-a]pyrazin-1(6H)- one | LC-MS (ESI): m/z (M + 1) 567.3, (M − 1) 565.3. |
| 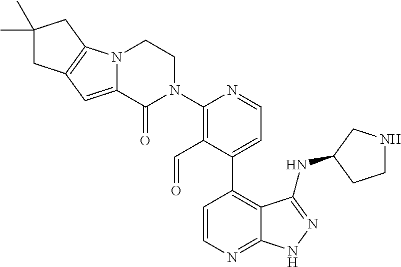 [SMILES: CC1(C)Cc2cc3n(c2C1)CCN(c1nccc(-c2ccnc4[nH]nc(N[C@@H]5CCNC5)c24)c1C=O)C3=O] | (R)-2-(7,7-dimethyl-1- oxo-1,3,4,6,7,8- hexahydro-2H- cyclopenta[4,5]pyrrolo [1,2-a]pyrazin-2-yl)-4- (3-(pyrrolidin-3- ylamino)-1H-pyrazolo [3,4-b]pyridin-4- yl)nicotinaldehyde | LC-MS (ESI): m/z (M + 1) 511.2, (M − 1) 509.2. |
| 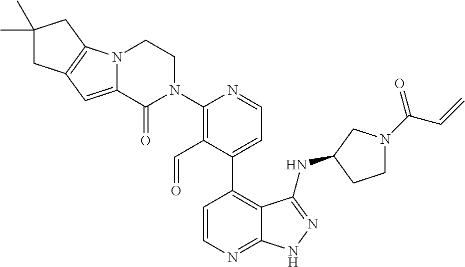 [SMILES: C=CC(=O)N1CC[C@@H](Nc2n[nH]c3nccc(-c4ccnc(N5CCn6c(cc7c6CC(C)(C)C7)C5=O)c4C=O)c23)C1] | (R)-4-(3-((1- acryloylpyrrolidin-3- yl)amino)-1H- pyrazolo[3,4-b]pyridin- 4-yl)-2-(7,7-dimethyl-1- oxo-1,3,4,6,7,8- hexahydro-2H- cylcopenta[4,5]pyrrolo [1,2-a]pyrazin-2- yl)nicotinaldehyde | LC-MS (ESI): m/z (M + 1) 565.2, (M − 1) 563.2. |

### Example 60: Synthesis of (R)-4-(tert-butyl)-N-(3-(3-(pyrrolidin-3-ylamino)-1H-pyrazolo[3,4-b]pyridin-4-yl)phenyl)benzamide and (R)—N-(3-(3-((1-acryloylpyrrolidin-3-yl)amino)-1H-pyrazolo[3,4-b]pyridin-4-yl)phenyl)-4-(tert-butyl)benzamide

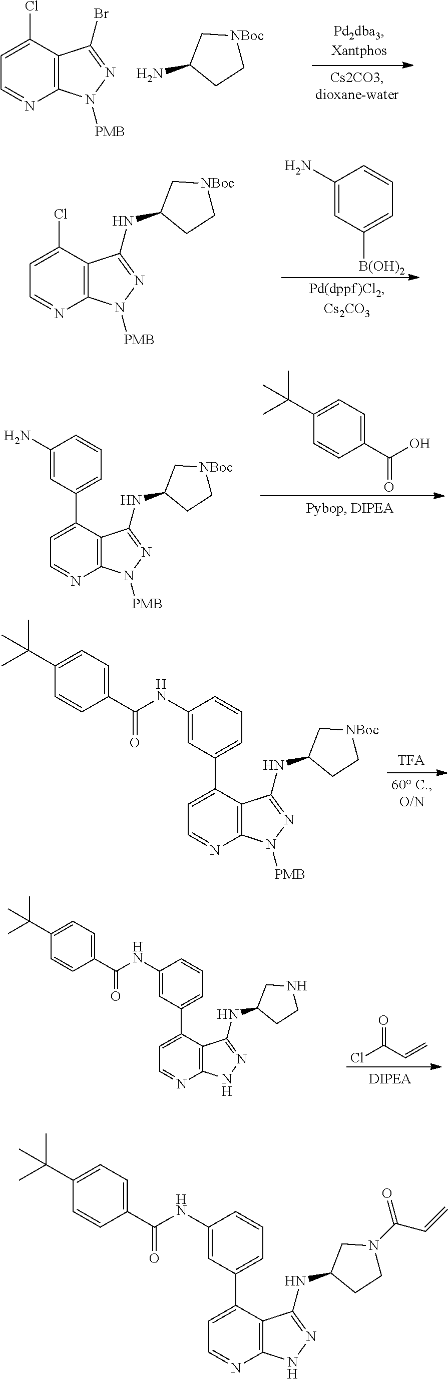

To a round bottom flask were added 3-bromo-4-chloro-1-(4-methoxybenzyl)-1H-pyrazolo[3,4-b]pyridine (10.0 g, 28.36 mmol), 1-methyl-1H-pyrazol-4-amine (6.87 g, 36.87 mmol), tris(dibenzylideneacetone)di-palladium(0) (0.815 g, 0.89 mmol), 4,5-Bis(diphenylphosphino)-9,9-dimethyl xanthene (1.03 g, 1.78 mmol), dioxane (125 ml) and cesium carbonate (18.4 g, 56.72 mmol). The reaction mixture was degassed with nitrogen for 2 minutes and was heated to 110° C. for 10 hours with stirring. After cooling to room temperature, the reaction mixture was filtered through a pad of celite followed by partitioning with ethyl acetate and brine. Separated organic layer, the aqueous layer was extracted with ethyl acetate twice. The combined organic layer was dried over MgSO₄. The solvent was evaporated and, the residue was purified by chromatography with 0-10% MeOH/DCM as eluent to give tert-butyl (R)-3-((4-chloro-1-(4-methoxybenzyl)-1H-pyrazolo[3,4-b]pyridin-3-yl)amino)pyrrolidine-1-carboxylate 11.8 g. LC/MS: MH⁺=458.1.

To a round bottom flask were added tert-butyl (R)-3-((4-chloro-1-(4-methoxybenzyl)-1H-pyrazolo[3,4-b]pyridin-3-yl)amino)pyrrolidine-1-carboxylate (1.0 g, 2.08 mmol), (3-aminophenyl)boronic acid (0.6 g, 4.4 mmol), 1,1′-Bis(diphenylphosphino)ferrocene]dichloropalladium(II), complex with dichloromethane (0.18 g 0.22 mmol), dioxance (20 ml), water (4 ml) and Cesium carbonate (1.42 g, 4.4 mmol). The reaction mixture was degassed with Nitrogen for 2 minutes and was heated to 110° C. for 2 hours with stirring. After cooling to room temperature, the reaction mixture was filtered through a pad of celite followed by partitioning with ethyl acetate and brine. Separated organic layer, the aqueous layer was extracted with ethyl acetate twice. The combined organic layer was dried over MgSO₄. The solvent was evaporated, and the residue was purified by chromatography with 0-10% MeOH/DCM as eluent to give tert-butyl (R)-3-((4-(3-aminophenyl)-1-(4-methoxybenzyl)-1H-pyrazolo[3,4-b]pyridin-3-yl)amino)pyrrolidine-1-carboxylate 1.04 g. LC/MS: MH⁺=515.2.

To a reaction mixture of tert-butyl (R)-3-((4-(3-aminophenyl)-1-(4-methoxybenzyl)-1H-pyrazolo[3,4-b]pyridin-3-yl)amino)pyrrolidine-1-carboxylate (300 mg, 0.58 mmol), 4-(tert-butyl)benzoic acid (104 mg, 0.58 mmol) and Pybop in DMF was added 151 mg DIPEA (151 mg, 1.16 mmol). The reaction mixture was stirred at room temperature overnight. It was partitioned with ethyl acetate and brine. Separated organic layer, the aqueous layer was extracted with ethyl acetate twice. The combined organic layer was dried over MgSO₄. Evaporated solvent, the residue was purified by chromatography with 30-90% ethyl acetate/Hex as eluent to give tert-butyl (R)-3-((4-(3-(4-(tert-butyl)benzamido) phenyl)-1-(4-methoxybenzyl)-1H-pyrazolo[3,4-b]pyridin-3-yl)amino) pyrrolidine-1-carboxylate 280 mg. LC/MS: MH⁺=675.3.

To a round bottom flask were added tert-butyl (R)-3-((4-(3-(4-(tert-butyl)benzamido)phenyl)-1-(4-methoxybenzyl)-1H-pyrazolo[3,4-b]pyridin-3-yl)amino)pyrrolidine-1-carboxylate and TFA (5 ml). The reaction mixture was heated to 50° C. overnight. When the reaction was complete, excess acid was removed under vacuum. The residue was neutralized with saturated sodium bicarbonate followed by extraction with ethyl acetate 3 times. The combined organic layer was dried over MgSO₄. Evaporated solvent, the residue was purified by chromatography with 0-10% MeOH/DCM as eluent to give (R)-4-(tert-butyl)-N-(3-(3-(pyrrolidin-3-ylamino)-1H-pyrazolo[3,4-b]pyridin-4-yl)phenyl)benzamide 125.0 mg. LC/MS: MH⁺=455.2.

To a solution of (R)-4-(tert-butyl)-N-(3-(3-(pyrrolidin-3-ylamino)-1H-pyrazolo[3,4-b]pyridin-4-yl)phenyl)benzamide (70.5 mg, 0.16 mmol) and DIPEA (61.3 mg, 0.47 mmol) in DCM was added a solution of acryloyl chloride (14.3 mg, 0.16 mmol) in THF at −78° C. dropwise. The reaction mixture was stirred at −78° C. for 10 min. The reaction mixture was quenched with water and adjusted pH to acidic to subject to reverse phase PreP HPLC. Desired ingredient was collected, filtered through basic PL-HCO3 MP resin and dried under lyophilyzation to give (R)—N-(3-(3-((1-acryloylpyrrolidin-3-yl)amino)-1H-pyrazolo[3,4-b]pyridin-4-yl)phenyl)-4-(tert-butyl)benzamide 15.8 mg. LC/MS: MH⁺=509.2. ¹H NMR (400 MHz, DMSO-d6) δ 10.05 (s, 1H), 8.34 (d, J=4.7 Hz, 1H), 7.98 (t, J=2.0 Hz, 1H), 7.88-7.73 (m, 2H), 7.71 (s, 0H), 7.51-7.33 (m, 3H), 7.28-7.11 (m, 1H), 6.86 (d, J=4.7 Hz, 1H), 5.97 (dd, J=16.8, 2.4 Hz, 1H), 5.45 (dd, J=10.5, 2.4 Hz, 1H), 4.24 (s, 2H), 3.70 (d, J=52.4 Hz, 1H), 3.40 (s, 4H), 2.10 (s, 1H), 1.26 (s, 10H).

The following compounds were prepared by analogous methods:

| Chemical Structure | Chemical Name | LC-MS, ¹H NMR |
|---|---|---|
| 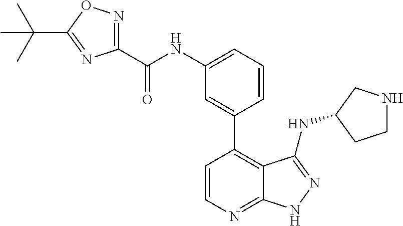 [SMILES: CC(C)(C)c1nc(C(=O)Nc2cccc(-c3ccnc4[nH]nc(N[C@H]5CCNC5)c34)c2)no1] | (S)-5-(tert-butyl)- N-(3-(3- (pyrrolidin-3-ylamino)- 1H-pyrazolo[3,4- b]pyridin-4-yl)phenyl)- 1,2,4-oxadiazole-3- carboxamidet | LC/MS: MH+ = 447.1 |
| 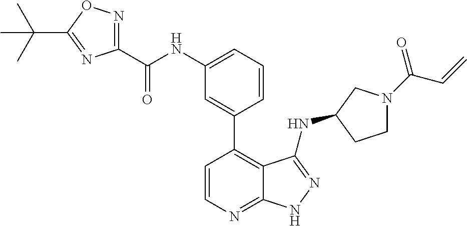 [SMILES: C=CC(=O)N1CC[C@@H](Nc2n[nH]c3nccc(-c4cccc(NC(=O)c5noc(C(C)(C)C)n5)c4)c23)C1] | (R)-N-(3-(3-((1- acryloylpyrrolidin3- yl)amino)-1H- pyrazolo[3,4- b]pyridin-4-yl)phenyl)- 5-(tertbutyl)- 1,2,4-oxadiazole-3- carboxamide | LC/MS: MH+ = 501.2; 1H NMR (400 MHz, DMSO-d6) δ 12.17 (s, 1H), 10.56 (s, 1H), 8.35 (d, J = 4.7 Hz, 1H), 7.93 (t, J = 2.0 Hz, 1H), 7.77 (dt, J = 8.2, 1.4 Hz, 1H), 7.46 (t, J = 7.9 Hz, 1H), 7.28 (dt, J = 7.7, 1.3 Hz, 1H), 6.87 (d, J = 4.7 Hz, 1H), 6.37 (s, 1H), 5.97 (dd, J = 16.8, 2.4 Hz, 1H), 5.47 (dd, J = 10.4, 2.4 Hz, 1H), 4.11 (s, 2H), 3.67 (d, J = 49.7 Hz, 1H), 3.33 (s, 4H), 2.08 (s, 1H), 1.74 (s, 1H), 1.40 (s, 9H). |
| 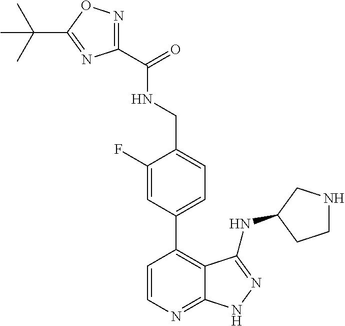 [SMILES: CC(C)(C)c1nc(C(=O)NCc2ccc(-c3ccnc4[nH]nc(N[C@@H]5CCNC5)c34)cc2F)no1] | (R)-5-(tert-butyl)-N- (2-fluoro-4- (3-(pyrrolidin-3- yloxy)-1Hpyrazolo [3,4-b]pyridin-4- yl)benzyl)-1,2,4- oxadiazole-3- carboxamide | LC/MS: MH+ = 480.3 |

### Example 61: Preparation of (R)-2,2,2-trifluoro-N-(1-(4-(4,4,5,5-tetramethyl-1,3,2-dioxaborolan-2-yl)phenyl) ethyl) acetamide

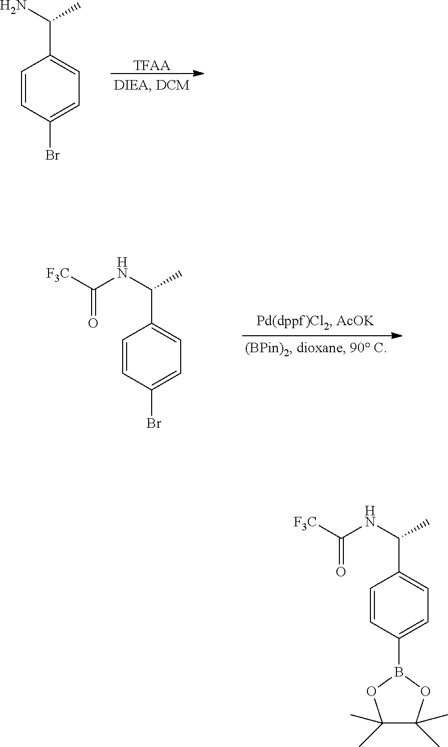

At 0° C., to solution of (R)-1-(4-bromophenyl)ethan-1-amine (20.0 g, 100 mmol) and DIEA (25.8 g, 200 mmol) in DCM (350 ml) was added TFAA (25.2 g, 130 mmol) dropwise. The mixture was stirred for 2 hr, and then was quenched with sat.aq.Na2CO3 and extracted with DCM. The combined organic layer was washed with brine, dried, filtered and concentrated to give the residue which was purified by flash chromatography on silica gel (0˜30% EA in PE) to afford (R)—N-(1-(4-bromophenyl)ethyl)-2,2,2-trifluoroacetamide (29.6 g, 4.71 mmol, 100%) as a white solid. LCMS (ESI) m/z (M+1)/(M+3): 296.10/298.11.

At 90° C., the suspension of (R)—N-(1-(4-bromophenyl)ethyl)-2,2,2-trifluoroacetamide (29.6 g, 100 mmol), KOAc (29.4 g, 300 mmol), _{PdCl2}(dppf)-CH₂Cl₂ adduct (2.5 g, 3 mmol) and 4,4,4′,4′,5,5,5′,5′-octamethyl-2,2′-bi(1,3,2-dioxaborolane) (27.9 g, 110 mmol) in 1,4-dioxane (350 ml) was stirred overnight under nitrogen atmosphere. The reaction mixture was cooled down to r.t. and filtered through a pad of Celite. The filtrate was washed with water and brine, dried over Na₂SO₄, filtered and concentrated. The crude product was purified by flash chromatography (0˜30% EA in PE) to provide (R)-2,2,2-trifluoro-N-(1-(4-(4,4,5,5-tetramethyl-1,3,2-dioxaborolan-2-yl) phenyl)ethyl) acetamide (34.0 g, 99.1% yield) as a white solid. LCMS (ESI) m/z (M+1): 344.41.

The following representative compounds were or can be prepared using the appropriate starting materials and reagents and following the procedures described herein.

TABLE 1
Representative compounds of the invention

| | | | | | Method |
|---|---|---|---|---|---|
| | | | | | of |
| | | | MS | | Prepara- |
| ID | Structure | MW | (Obsvd)* | Name | tion |
| 1 | 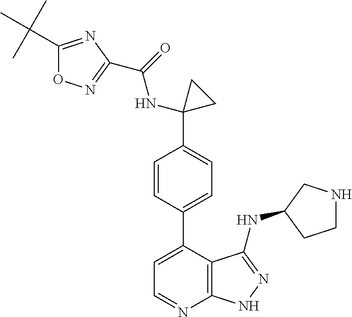 [SMILES: CC(C)(C)c1nc(C(=O)NC2(c3ccc(-c4ccnc5[nH]nc(N[C@@H]6CCNC6)c45)cc3)CC2)no1] | 486.58 | See Ex- amples | 5-tert-butyl-N-{1-[4-(3-{[(3R)- pyrrolidin-3-yl]amino}-1H- pyrazolo[3,4-b]pyridin-4- yl)phenyl]cyclopropyl}-1,2,4- oxadiazole-3-carboxamide | See Ex- amples |
| 2 | 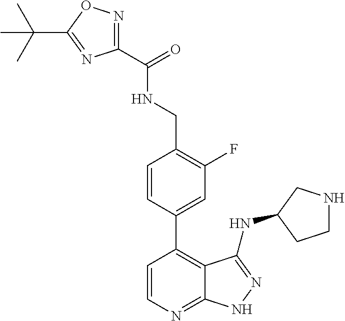 [SMILES: CC(C)(C)c1nc(C(=O)NCc2ccc(-c3ccnc4[nH]nc(N[C@@H]5CCNC5)c34)cc2F)no1] | 478.53 | See Ex- amples | 5-tert-butyl-N-{[2-fluoro- 4-(3-{[(3R)- pyrrolidin-3-yl]amino}-1H- pyrazolo[3,4-b]pyridin-4- yl)phenyl]methyl}-1,2,4- oxadiazole- 3-carboxamide | See Ex- amples |
| 3 | 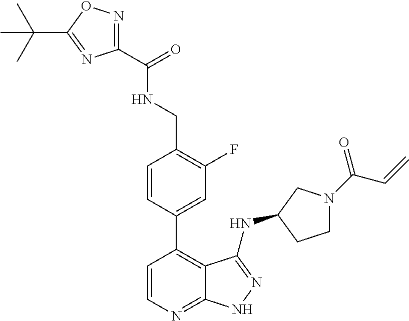 [SMILES: C=CC(=O)N1CC[C@@H](Nc2n[nH]c3nccc(-c4ccc(CNC(=O)c5noc(C(C)(C)C)n5)c(F)c4)c23)C1] | 532.58 | See Ex- amples | 5-tert-butyl-N-{[2-fluoro- 4-(3-{[(3R)- 1-(prop-2-enoyl)pyrrolidin-3- yl]amino}-1H- pyrazolo[3,4-b]pyridin- 4-yl)phenyl]methyl}-1,2,4- oxadiazole-3-carboxamide | See Ex- amples |
| 4 | 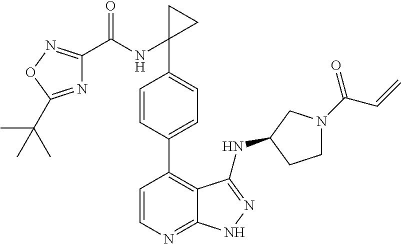 [SMILES: C=CC(=O)N1CC[C@@H](Nc2n[nH]c3nccc(-c4ccc(C5(NC(=O)c6noc(C(C)(C)C)n6)CC5)cc4)c23)C1] | 540.63 | See Ex- amples | 5-tert-butyl-N-{1-[4- (3-{[(3R)-1-(prop- 2-enoyl)pyrrolidin- 3-yl]amino}-1H- pyrazolo[3,4-b]pyridin-4- yl)phenyl]cyclopropyl}-1,2,4- oxadiazole-3-carboxamide | See Ex- amples |
| 5 | 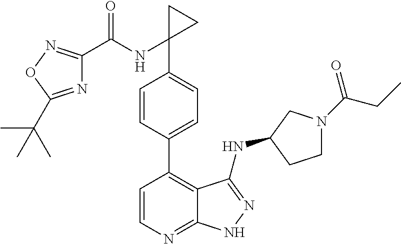 [SMILES: CCC(=O)N1CC[C@@H](Nc2n[nH]c3nccc(-c4ccc(C5(NC(=O)c6noc(C(C)(C)C)n6)CC5)cc4)c23)C1] | 542.64 | See Ex- amples | 5-tert-butyl-N-{1-[4-(3-{[(3R)-1- propanoylpyrrolidin- 3-yl]amino}-1H- pyrazolo[3,4-b]pyridin-4- yl)phenyl]cyclopropyl}-1,2,4- oxadiazole-3-carboxamide | See Ex- amples |
| 6 | 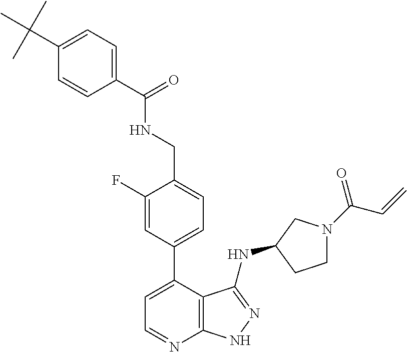 [SMILES: C=CC(=O)N1CC[C@@H](Nc2n[nH]c3nccc(-c4ccc(CNC(=O)c5ccc(C(C)(C)C)cc5)c(F)c4)c23)C1] | 540.64 | See Ex- amples | 4-tert-butyl-N-{[2- fluoro-4-(3-{[(3R)- 1-(prop-2-enoyl)pyrrolidin-3- yl]amino}-1H- pyrazolo[3,4-b]pyridin- 4-yl)phenyl]methyl}benzamide | See Ex- amples |
| 7 | 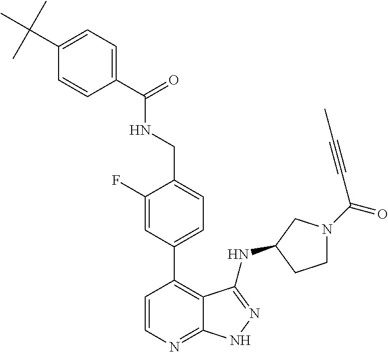 [SMILES: CC#CC(=O)N1CC[C@@H](Nc2n[nH]c3nccc(-c4ccc(CNC(=O)c5ccc(C(C)(C)C)cc5)c(F)c4)c23)C1] | 552.65 | See Ex- amples | N-{[4-(3-{[(3R)-1-(but-2- ynoyl)pyrrolidin-3-yl]amino}-1H- pyrazolo[3,4-b]pyridin-4-yl)-2- fluorophenyl]methyl}-4-tert- butylbenzamide | See Ex- amples |
| 8 | 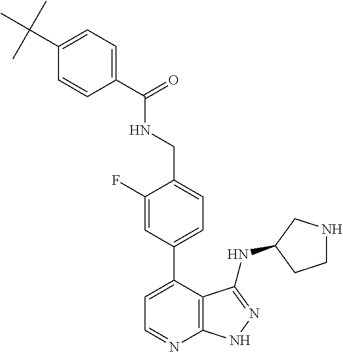 [SMILES: CC(C)(C)c1ccc(C(=O)NCc2ccc(-c3ccnc4[nH]nc(N[C@@H]5CCNC5)c34)cc2F)cc1] | 486.60 | See Ex- amples | 4-tert-butyl-N-{[2-fluoro- 4-(3-{[(3R)- pyrrolidin-3-yl]amino}-1H- pyrazolo[3,4-b]pyridin-4- yl)phenyl]methyl}benzamide | See Ex- amples |
| 9 | 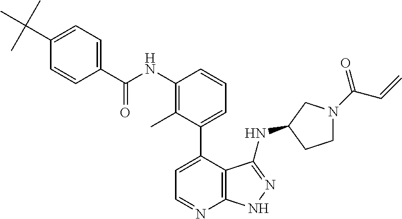 [SMILES: C=CC(=O)N1CC[C@@H](Nc2n[nH]c3nccc(-c4cccc(NC(=O)c5ccc(C(C)(C)C)cc5)c4C)c23)C1] | 522.65 | See Ex- amples | 4-tert-butyl-N-[2-methyl- 3-(3-{[(3R)- 1-(prop-2-enoyl)pyrrolidin-3- yl]amino}-1H- pyrazolo[3,4-b]pyridin- 4-yl)phenyl]benzamide | See Ex- amples |
| 10 | 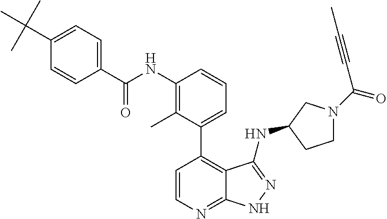 [SMILES: CC#CC(=O)N1CC[C@@H](Nc2n[nH]c3nccc(-c4cccc(NC(=O)c5ccc(C(C)(C)C)cc5)c4C)c23)C1] | 534.66 | See Ex- amples | N-[3-(3-{[(3R)-1-(but-2- ynoyl)pyrrolidin-3-yl]amino}-1H- pyrazolo[3,4-b]pyridin-4-yl)-2- methylphenyl]-4- tert-butylbenzamide | See Ex- amples |
| 11 | 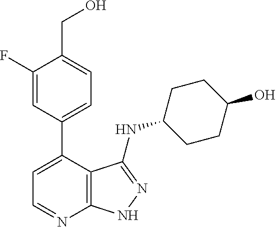 [SMILES: OCc1ccc(-c2ccnc3[nH]nc(N[C@H]4CC[C@H](O)CC4)c23)cc1F] | 356.40 | See Ex- amples | (1r,4r)-4-({4-[3-fluoro-4- (hydroxymethyl)phenyl]-1H- pyrazolo[3,4-b]pyridin-3- yl}amino)cyclohexan-1-ol | See Ex- amples |
| 12 | 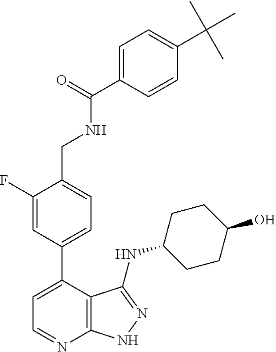 [SMILES: CC(C)(C)c1ccc(C(=O)NCc2ccc(-c3ccnc4[nH]nc(N[C@H]5CC[C@H](O)CC5)c34)cc2F)cc1] | 515.63 | See Ex- amples | 4-tert-butyl-N-{[2-fluoro-4-(3- {[(1r,4r)-4-hydroxy- cyclohexyl]amino}- 1H-pyrazolo[3,4-b]pyridin-4- yl)phenyl]methyl}benzamide | See Ex- amples |
| 13 | 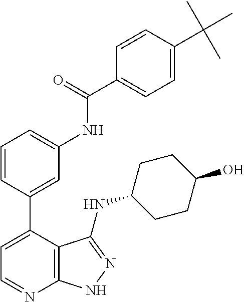 [SMILES: CC(C)(C)c1ccc(C(=O)Nc2cccc(-c3ccnc4[nH]nc(N[C@H]5CC[C@H](O)CC5)c34)c2)cc1] | 483.62 | See Ex- amples | 4-tert-butyl-N-[3-(3-{[(1r,4r)-4- hydroxycyclohexyl]amino}-1H- pyrazolo[3,4-b]pyridin-4- yl)phenyl]benzamide | See Ex- amples |
| 14 | 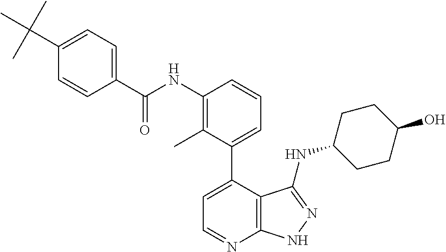 [SMILES: Cc1c(NC(=O)c2ccc(C(C)(C)C)cc2)cccc1-c1ccnc2[nH]nc(N[C@H]3CC[C@H](O)CC3)c12] | 497.64 | See Ex- amples | 4-tert-butyl-N-[2-methyl- 3-(3-{[(1r,4r)- 4-hydroxycyclohexyl]amino}-1H- pyrazolo[3,4-b]pyridin-4- yl)phenyl]benzamide | See Ex- amples |
| 15 | 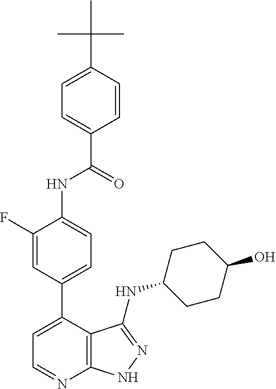 [SMILES: CC(C)(C)c1ccc(C(=O)Nc2ccc(-c3ccnc4[nH]nc(N[C@H]5CC[C@H](O)CC5)c34)cc2F)cc1] | 501.61 | See Ex- amples | 4-tert-butyl-N-[2-fluoro- 4-(3-{[(1r,4r)- 4-hydroxycyclohexyl]amino}-1H- pyrazolo[3,4-b]pyridin-4- yl)phenyl]benzamide | See Ex- amples |
| 16 | 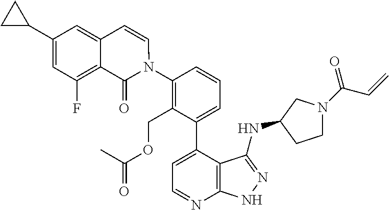 [SMILES: C=CC(=O)N1CC[C@@H](Nc2n[nH]c3nccc(-c4cccc(-n5ccc6cc(C7CC7)cc(F)c6c5=O)c4COC(C)=O)c23)C1] | 606.66 | See Ex- amples | [2-(6-cyclopropyl-8-fluoro- 1-oxo-1,2-dihydroisoquinolin- 2-yl)-6-(3-{[(3R)- 1-(prop-2-enoyl)pyrrolidin-3- yl]amino}-1H-pyrazolo[3,4-b] pyridin-4-yl)phenyl]methyl acetate | See Ex- amples |
| 17 | 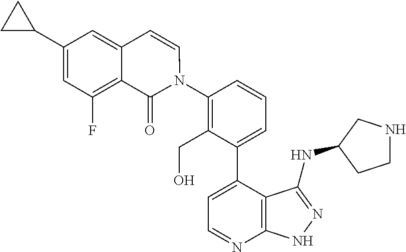 [SMILES: O=c1c2c(F)cc(C3CC3)cc2ccn1-c1cccc(-c2ccnc3[nH]nc(N[C@@H]4CCNC4)c23)c1CO] | 510.57 | See Ex- amples | 6-cyclopropyl-8-fluoro-2-[2- (hydroxymethyl)-3-(3-{[(3R)- pyrrolidin-3-yl]amino}-1H- pyrazolo[3,4-b]pyridin- 4-yl)phenyl]- 1,2-dihydroisoquinolin-1-one | See Ex- amples |
| 18 | 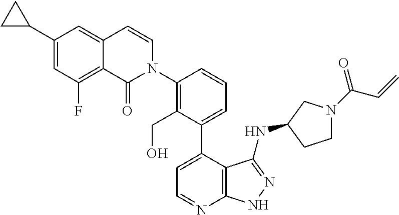 [SMILES: C=CC(=O)N1CC[C@@H](Nc2n[nH]c3nccc(-c4cccc(-n5ccc6cc(C7CC7)cc(F)c6c5=O)c4CO)c23)C1] | 564.62 | See Ex- amples | 6-cyclopropyl-8-fluoro-2-[2- (hydroxymethyl)-3- (3-{[(3R)-1-(prop-2-enoyl) pyrrolidin-3-yl]amino}-1H- pyrazolo[3,4-b]pyridin- 4-yl)phenyl]- 1,2-dihydroisoquinolin-1-one | See Ex- amples |
| 19 | 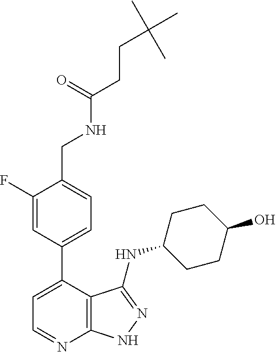 [SMILES: CC(C)(C)CCC(=O)NCc1ccc(-c2ccnc3[nH]nc(N[C@H]4CC[C@H](O)CC4)c23)cc1F] | 467.59 | See Ex- amples | N-{[2-fluoro-4-(3-{[(1r,4r)-4- hydroxycyclohexyl]amino}-1H- pyrazolo[3,4-b]pyridin-4- yl)phenyl]methyl}-4,4- dimethylpentanamide | See Ex- amples |
| 20 | 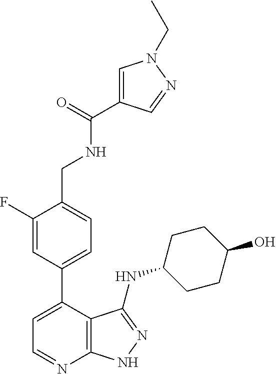 [SMILES: CCn1cc(C(=O)NCc2ccc(-c3ccnc4[nH]nc(N[C@H]5CC[C@H](O)CC5)c34)cc2F)cn1] | 477.54 | See Ex- amples | 1-ethyl-N-{[2-fluoro- 4-(3-{[(1r,4r)-4- hydroxycyclohexyl]amino}-1H- pyrazolo[3,4-b]pyridin-4- yl)phenyl]methyl}-1H-pyrazole-4- carboxamide | See Ex- amples |
| 21 | 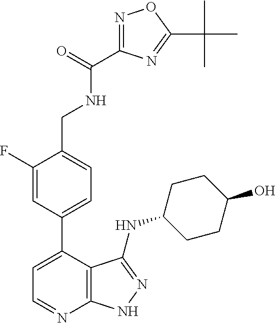 [SMILES: CC(C)(C)c1nc(C(=O)NCc2ccc(-c3ccnc4[nH]nc(N[C@H]5CC[C@H](O)CC5)c34)cc2F)no1] | 507.57 | See Ex- amples | 5-tert-butyl-N-{[2-fluoro-4-(3- {[(1r,4r)-4- hydroxycyclohexyl]amino}- 1H-pyrazolo[3,4-b]pyridin-4- yl)phenyl]methyl}-1,2,4- oxadiazole-3-carboxamide | See Ex- amples |
| 22 | 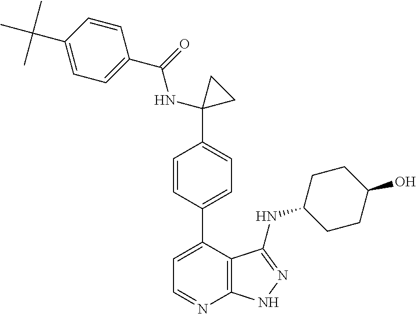 [SMILES: CC(C)(C)c1ccc(C(=O)NC2(c3ccc(-c4ccnc5[nH]nc(N[C@H]6CC[C@H](O)CC6)c45)cc3)CC2)cc1] | 523.68 | See Ex- amples | 4-tert-butyl-N-{1-[4-(3-{[(1r,4r-4- hydroxycyclohexyl]amino}-1H- pyrazolo[3,4-b]pyridin-4- yl)phenyl]cyclopropyl}benzamide | See Ex- amples |
| 23 | 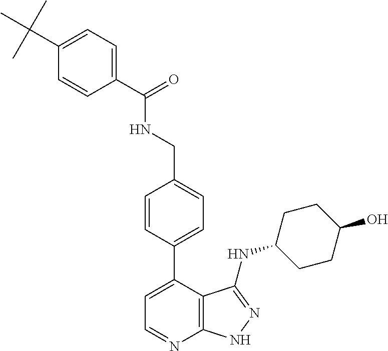 [SMILES: CC(C)(C)c1ccc(C(=O)NCc2ccc(-c3ccnc4[nH]nc(N[C@H]5CC[C@H](O)CC5)c34)cc2)cc1] | 497.64 | See Ex- amples | 4-tert-butyl-N-{[4-(3-{[(1r,4r)-4- hydroxycyclohexyl]amino}-1H- pyrazolo[3,4-b]pyridin-4- yl)phenyl]methyl}benzamide | See Ex- amples |
| 24 | 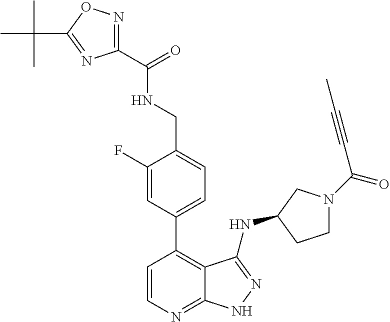 [SMILES: CC#CC(=O)N1CC[C@@H](Nc2n[nH]c3nccc(-c4ccc(CNC(=O)c5noc(C(C)(C)C)n5)c(F)c4)c23)C1] | 544.59 | See Ex- amples | N-{[4-(3-{[(3R)-1-(but-2- ynoyl)pyrrolidin-3-yl]amino}-1H- pyrazolo[3,4-b]pyridin-4-yl)-2- fluorophenyl]methyl}-5-tert-butyl- 1,2,4-oxadiazole-3-carboxamide | Method C |
| 25 | 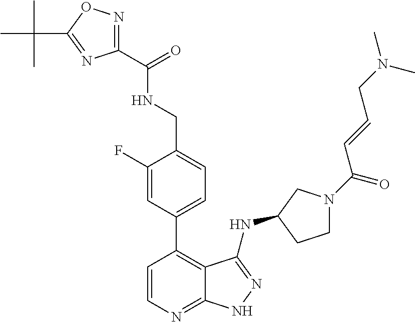 [SMILES: CN(C)C/C=C/C(=O)N1CC[C@@H](Nc2n[nH]c3nccc(-c4ccc(CNC(=O)c5noc(C(C)(C)C)n5)c(F)c4)c23)C1] | 589.68 | See Ex- amples | 5-tert-butyl-N-{[4- (3-{[(3R)-1-[(2E)- 4-(dimethylamino)but-2- enoyl]pyrrolidin-3-yl]amino}-1H- pyrazolo[3,4-b]pyridin-4-yl)-2- fluorophenyl]methyl}-1,2,4- oxadiazole-3-carboxamide | Method C |
| 26 | 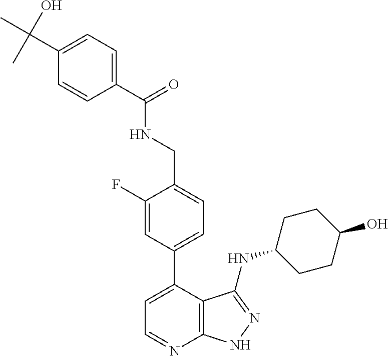 [SMILES: CC(C)(O)c1ccc(C(=O)NCc2ccc(-c3ccnc4[nH]nc(N[C@H]5CC[C@H](O)CC5)c34)cc2F)cc1] | 517.61 | See Ex- amples | N-{[2-fluoro-4-(3-{[(1r,4r)-4- hydroxycyclohexyl]amino}-1H- pyrazolo[3,4-b]pyridin-4- yl)phenyl]methyl}-4-(2- hydroxypropan-2-yl)benzamide | See Ex- amples |
| 27 | 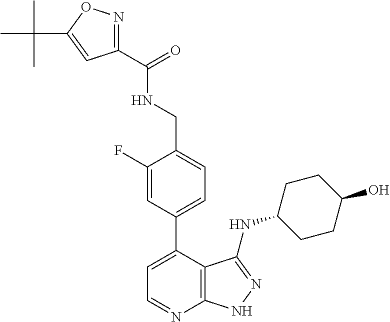 [SMILES: CC(C)(C)c1cc(C(=O)NCc2ccc(-c3ccnc4[nH]nc(N[C@H]5CC[C@H](O)CC5)c34)cc2F)no1] | 506.58 | See Ex- amples | 5-tert-butyl-N-{[2-fluoro-4-(3- {[(1r,4r)-4- hydroxycyclohexyl]amino}- 1H-pyrazolo[3,4-b]pyridin-4- yl)phenyl]methyl}-1,2-oxazole-3- carboxamide | See Ex- amples |
| 28 | 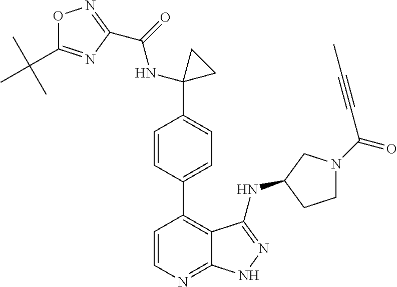 [SMILES: CC#CC(=O)N1CC[C@@H](Nc2n[nH]c3nccc(-c4ccc(C5(NC(=O)c6noc(C(C)(C)C)n6)CC5)cc4)c23)C1] | 552.64 | See Ex- amples | N-{1-[4-(3-{[(3R)-1-(but-2- ynoyl)pyrrolidin-3-yl]amino}-1H- pyrazolo[3,4-b]pyridin-4- yl)phenyl]cyclopropyl}- 5-tert-butyl- 1,2,4-oxadiazole-3-carboxamide | See Ex- amples |
| 29 | 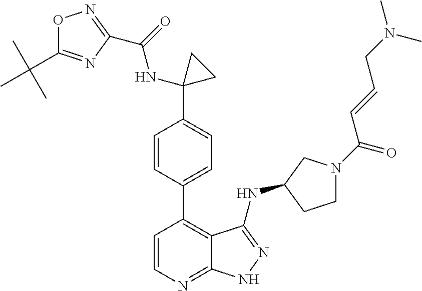 [SMILES: CN(C)C/C=C/C(=O)N1CC[C@@H](Nc2n[nH]c3nccc(-c4ccc(C5(NC(=O)c6noc(C(C)(C)C)n6)CC5)cc4)c23)C1] | 597.72 | See Ex- amples | 5-tert-butyl-N-{1-[4- (3-{[(3R)-1-[(2E)- 4-(dimethylamino)but-2- enoyl]pyrrolidin-3-yl]amino}-1H- pyrazolo[3,4-b]pyridin-4- yl)phenyl]cyclopropyl}-1,2,4- oxadiazole-3-carboxamide | See Ex- amples |
| 30 | 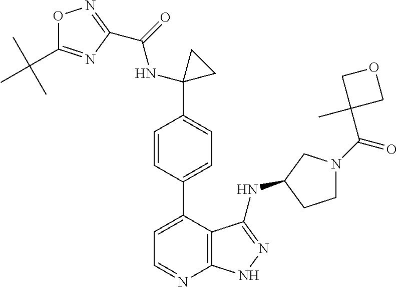 [SMILES: CC1(C(=O)N2CC[C@@H](Nc3n[nH]c4nccc(-c5ccc(C6(NC(=O)c7noc(C(C)(C)C)n7)CC6)cc5)c34)C2)COC1] | 584.68 | See Ex- amples | 5-tert-butyl-N-{1-[4- (3-{[(3R)-1-(3- methyloxetane-3- carbonyl)pyrrolidin- 3-yl]amino}-1H-pyrazolo[3,4- b]pyridin-4-yl) phenyl]cyclopropyl}- 1,2,4-oxadiazole-3-carboxamide | See Ex- amples |
| 31 | 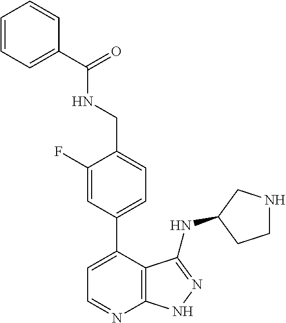 [SMILES: O=C(NCc1ccc(-c2ccnc3[nH]nc(N[C@@H]4CCNC4)c23)cc1F)c1ccccc1] | 430.49 | See Ex- amples | N-{[2-fluoro-4-(3-{[(3R)- pyrrolidin-3-yl]amino}- 1H-pyrazolo[3,4-b]pyridin- 4-yl)phenyl]methyl}benzamide | See Ex- amples |
| 32 | 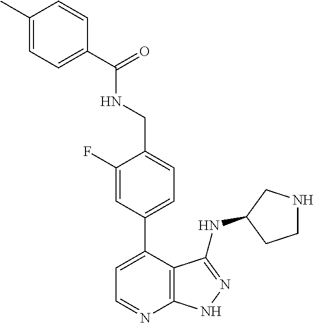 [SMILES: Cc1ccc(C(=O)NCc2ccc(-c3ccnc4[nH]nc(N[C@@H]5CCNC5)c34)cc2F)cc1] | 444.51 | See Ex- amples | N-{[2-fluoro-4-(3-{[(3R)- pyrrolidin-3-yl]amino}- 1H-pyrazolo[3,4-b]pyridin- 4-yl)phenyl]methyl}-4- methylbenzamide | See Ex- amples |
| 33 | 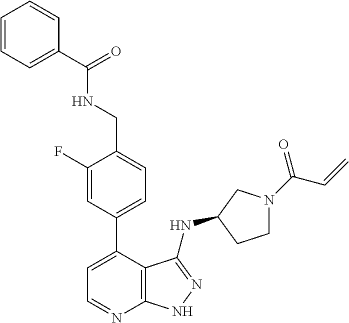 [SMILES: C=CC(=O)N1CC[C@@H](Nc2n[nH]c3nccc(-c4ccc(CNC(=O)c5ccccc5)c(F)c4)c23)C1] | 484.54 | See Ex- amples | N-{[2-fluoro-4- (3-{[(3R)-1-(prop-2- enoyl)pyrrolidin-3-yl]amino}-1H- pyrazolo[3,4-b]pyridin-4- yl)phenyl]methyl}benzamide | See Ex- amples |
| 34 | 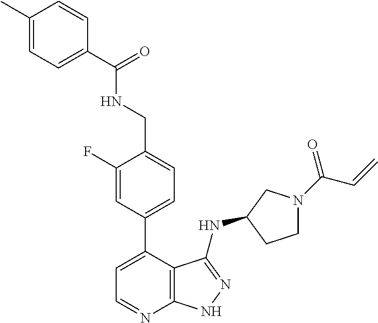 [SMILES: C=CC(=O)N1CC[C@@H](Nc2n[nH]c3nccc(-c4ccc(CNC(=O)c5ccc(C)cc5)c(F)c4)c23)C1] | 498.56 | See Ex- amples | N-{[2-fluoro-4- (3-{[(3R)-1-(prop-2- enoyl)pyrrolidin-3-yl]amino}-1H- pyrazolo[3,4-b]pyridin-4- yl)phenyl]methyl}-4- methylbenzamide | See Ex- amples |
| 35 | 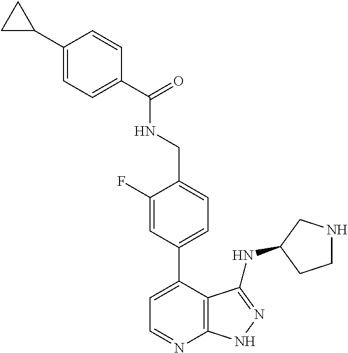 [SMILES: O=C(NCc1ccc(-c2ccnc3[nH]nc(N[C@@H]4CCNC4)c23)cc1F)c1ccc(C2CC2)cc1] | 470.55 | See Ex- amples | 4-cyclopropyl-N-{[2-fluoro-4-(3- {[(3R)-pyrrolidin-3-yl]amino}-1H- pyrazolo[3,4-b]pyridin-4- yl)phenyl]methyl}benzamide | See Ex- amples |
| 36 | 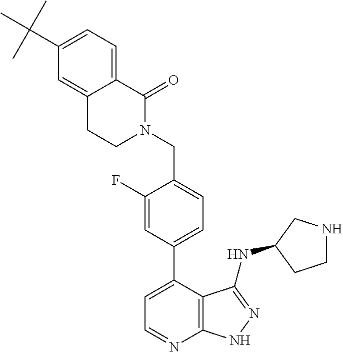 [SMILES: CC(C)(C)c1ccc2c(c1)CCN(Cc1ccc(-c3ccnc4[nH]nc(N[C@@H]5CCNC5)c34)cc1F)C2=O] | 512.63 | See Ex- amples | 6-tert-butyl-2-{[2- fluoro-4-(3-{[(3R)- pyrrolidin-3-yl]amino}-1H- pyrazolo[3,4-b]pyridin-4- yl)phenyl]methyl}-1,2,3,4- tetrahydroisoquinolin-1-one | See Ex- amples |
| 37 | 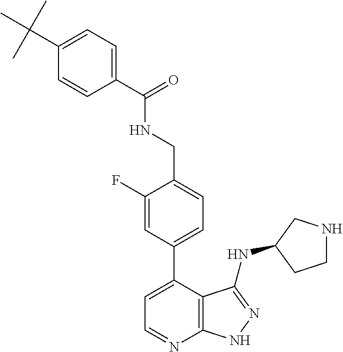 [SMILES: CC(C)(C)c1ccc(C(=O)NCc2ccc(-c3ccnc4[nH]nc(N[C@@H]5CCNC5)c34)cc2F)cc1] | 486.60 | See Ex- amples | 4-tert-butyl-N-{[2- fluoro-4-(3-{[(3S)- pyrrolidin-3-yl]amino}-1H- pyrazolo[3,4-b]pyridin-4- yl)phenyl]methyl}benzamide | See Ex- amples |
| 38 | 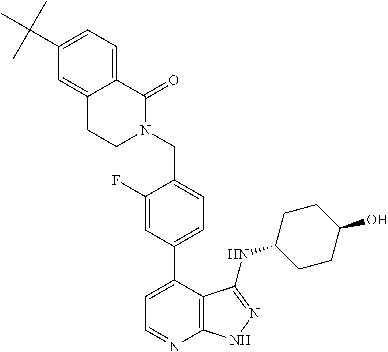 [SMILES: CC(C)(C)c1ccc2c(c1)CCN(Cc1ccc(-c3ccnc4[nH]nc(N[C@H]5CC[C@H](O)CC5)c34)cc1F)C2=O] | 541.67 | See Ex- amples | 6-tert-butyl-2-{[2-fluoro- 4-(3-{[(1r,4r)- 4-hydroxycyclohexyl]amino}-1H- pyrazolo[3,4-b]pyridin-4- yl)phenyl]methyl}-1,2,3,4- tetrahydroisoquinolin-1-one | See Ex- amples |
| 39 | 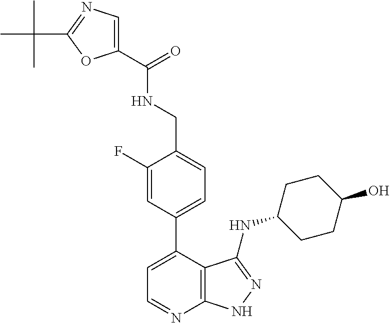 [SMILES: CC(C)(C)c1ncc(C(=O)NCc2ccc(-c3ccnc4[nH]nc(N[C@H]5CC[C@H](O)CC5)c34)cc2F)o1] | 506.58 | See Ex- amples | 2-tert-butyl-N-{[2-fluoro-4-(3- {[(1r,4r)-4- hydroxycyclohexyl]amino}- 1H-pyrazolo[3,4-b]pyridin-4- yl)phenyl]methyl}-1,3-oxazole-5- carboxamide | See Ex- amples |
| 40 | 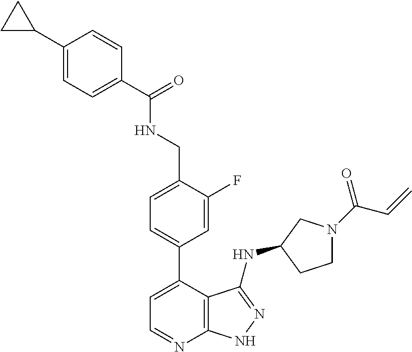 [SMILES: C=CC(=O)N1CC[C@@H](Nc2n[nH]c3nccc(-c4ccc(CNC(=O)c5ccc(C6CC6)cc5)c(F)c4)c23)C1] | 524.60 | See Ex- amples | 4-cyclopropyl-N-{[2-fluoro-4-(3- {[(3R)-1-(prop-2-enoyl)pyrrolidin- 3-yl]amino}-1H- pyrazolo[3,4-b]pyridin- 4-yl)phenyl]methyl}benzamide | See Ex- amples |
| 41 | 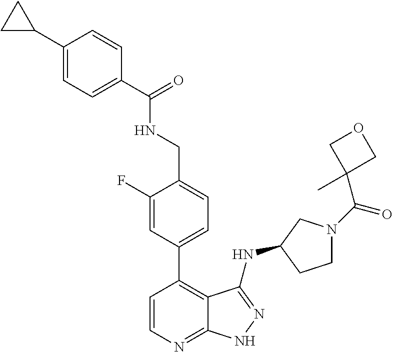 [SMILES: CC1(C(=O)N2CC[C@@H](Nc3n[nH]c4nccc(-c5ccc(CNC(=O)c6ccc(C7CC7)cc6)c(F)c5)c34)C2)COC1] | 568.65 | See Ex- amples | 4-cyclopropyl-N-{[2-fluoro-4-(3- {[(3R)-1-(3-methyloxetane-3- carbonyl)pyrrolidin-3-yl]amino}- 1H-pyrazolo[3,4-b]pyridin-4- yl)phenyl]methyl}benzamide | See Ex- amples |
| 42 | 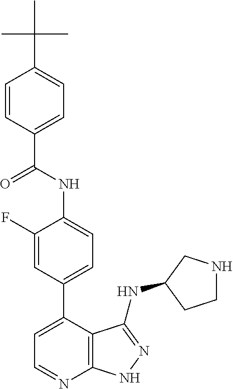 [SMILES: CC(C)(C)c1ccc(C(=O)Nc2ccc(-c3ccnc4[nH]nc(N[C@@H]5CCNC5)c34)cc2F)cc1] | 472.57 | See Ex- amples | 4-tert-butyl-N-[2-fluoro- 4-(3-{[(3R)- pyrrolidin-3-yl]amino}-1H- pyrazolo[3,4-b]pyridin-4- yl)phenyl]benzamide | See Ex- amples |
| 43 | 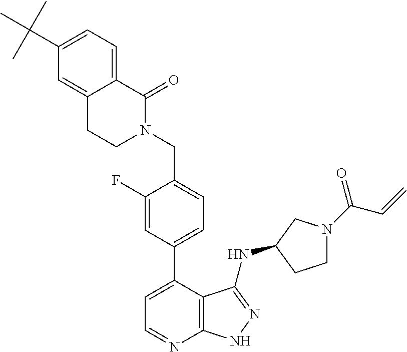 [SMILES: C=CC(=O)N1CC[C@@H](Nc2n[nH]c3nccc(-c4ccc(CN5CCc6cc(C(C)(C)C)ccc6C5=O)c(F)c4)c23)C1] | 566.68 | See Ex- amples | 6-tert-butyl-2-{[2-fluoro- 4-(3-{[(3R)-1-(prop- 2-enoyl)pyrrolidin-3-yl]amino}- 1H-pyrazolo[3,4-b]pyridin-4- yl)phenyl]methyl}-1,2,3,4- tetrahydroisoquinolin-1-one | See Ex- amples |
| 44 | 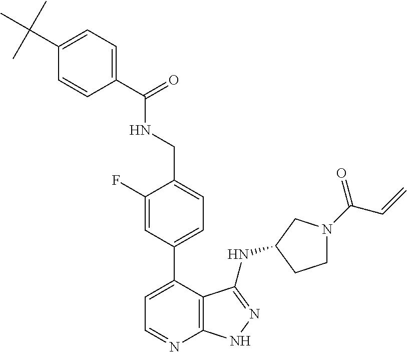 [SMILES: C=CC(=O)N1CC[C@H](Nc2n[nH]c3nccc(-c4ccc(CNC(=O)c5ccc(C(C)(C)C)cc5)c(F)c4)c23)C1] | 540.64 | See Ex- amples | 4-tert-butyl-N-{[2-fluoro- 4-(3-{[(3S)-1-(prop-2- enoyl)pyrrolidin-3-yl]amino}- 1H-pyrazolo[3,4-b]pyridin- 4-yl)phenyl]methyl}benzamide | See Ex- amples |
| 45 | 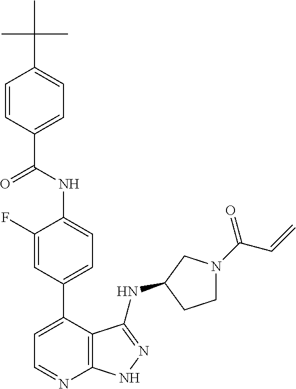 [SMILES: C=CC(=O)N1CC[C@@H](Nc2n[nH]c3nccc(-c4ccc(NC(=O)c5ccc(C(C)(C)C)cc5)c(F)c4)c23)C1] | 526.62 | See Ex- amples | 4-tert-butyl-N-[2-fluoro- 4-(3-{[(3R)-1-(prop-2- enoyl)pyrrolidin-3-yl]amino}- 1H-pyrazolo[3,4-b]pyridin-4- yl)phenyl]benzamide | See Ex- amples |
| 46 | 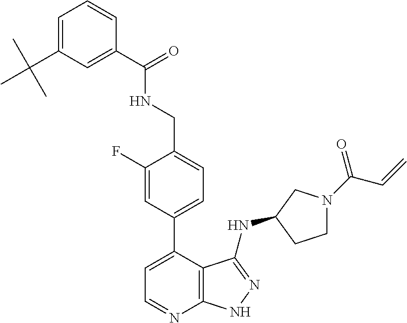 [SMILES: C=CC(=O)N1CC[C@@H](Nc2n[nH]c3nccc(-c4ccc(CNC(=O)c5cccc(C(C)(C)C)c5)c(F)c4)c23)C1] | 540.64 | See Ex- amples | 3-tert-butyl-N-{[2-fluoro- 4-(3-{[(3R)- 1-(prop-2-enoyl)pyrrolidin-3- yl]amino}-1H- pyrazolo[3,4-b]pyridin- 4-yl)phenyl]methyl}benzamide | See Ex- amples |
| 47 | 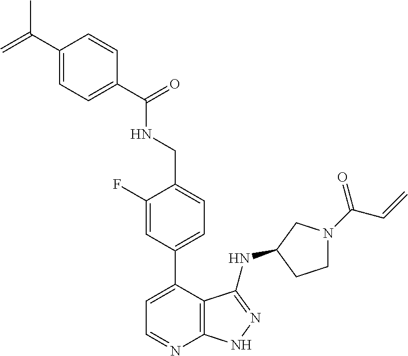 [SMILES: C=CC(=O)N1CC[C@@H](Nc2n[nH]c3nccc(-c4ccc(CNC(=O)c5ccc(C(=C)C)cc5)c(F)c4)c23)C1] | 524.60 | See Ex- amples | N-{[2-fluoro-4- (3-{[(3R)-1-(prop-2- enoyl)pyrrolidin-3-yl]amino}-1H- pyrazolo[3,4-b]pyridin-4- yl)phenyl]methyl}-4-(prop-1-en-2- yl)benzamide | See Ex- amples |
| 48 | 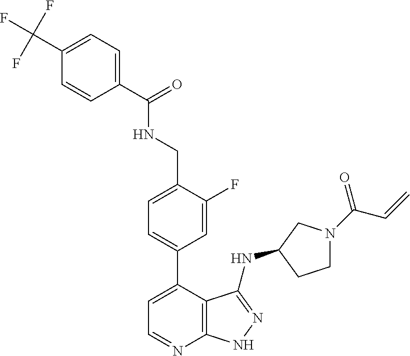 [SMILES: C=CC(=O)N1CC[C@@H](Nc2n[nH]c3nccc(-c4ccc(CNC(=O)c5ccc(C(F)(F)F)cc5)c(F)c4)c23)C1] | 552.53 | See Ex- amples | N-{[2-fluoro-4- (3-{[(3R)-1-(prop-2- enoyl)pyrrolidin-3-yl]amino}-1H- pyrazolo[3,4-b]pyridin-4- yl)phenyl]methyl}-4- (trifluoromethyl)benzamide | See Ex- amples |
| 49 | 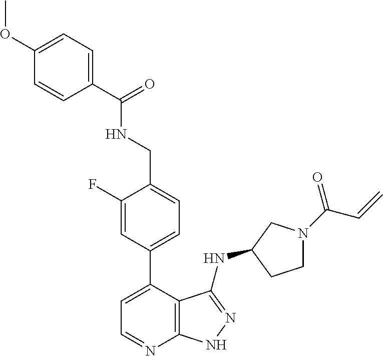 [SMILES: C=CC(=O)N1CC[C@@H](Nc2n[nH]c3nccc(-c4ccc(CNC(=O)c5ccc(OC)cc5)c(F)c4)c23)C1] | 514.56 | See Ex- amples | N-{[2-fluoro-4- (3-{[(3R)-1-(prop-2- enoyl)pyrrolidin-3-yl]amino}-1H- pyrazolo[3,4-b]pyridin-4- yl)phenyl]methyl}-4- methoxybenzamide | See Ex- amples |
| 50 | 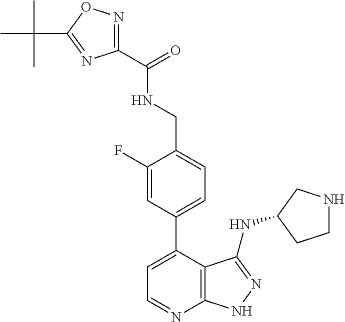 [SMILES: CC(C)(C)c1nc(C(=O)NCc2ccc(-c3ccnc4[nH]nc(N[C@H]5CCNC5)c34)cc2F)no1] | 478.53 | See Ex- amples | 5-tert-butyl-N-{[2-fluoro- 4-(3-{[(3S)- pyrrolidin-3-yl]amino}-1H- pyrazolo[3,4-b]pyridin-4- yl)phenyl]methyl}-1,2,4- oxadiazole-3-carboxamide | See Ex- amples |
| 51 | 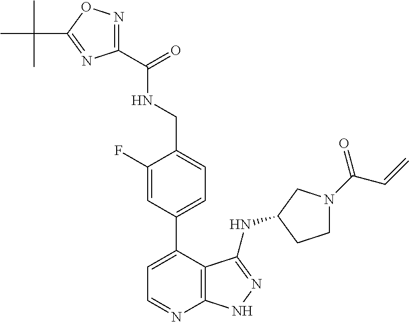 [SMILES: C=CC(=O)N1CC[C@H](Nc2n[nH]c3nccc(-c4ccc(CNC(=O)c5noc(C(C)(C)C)n5)c(F)c4)c23)C1] | 532.58 | 533.2 | 5-tert-butyl-N-{[2-fluoro- 4-(3-{[(3S)- 1-(prop-2-enoyl)pyrrolidin-3- yl]amino}-1H- pyrazolo[3,4-b]pyridin-4-yl) phenyl]methyl}-1,2,4-oxadiazole- 3-carboxamide | Method E |
| 52 | 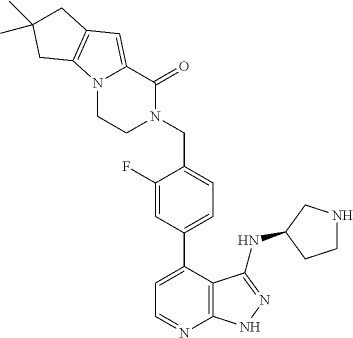 [SMILES: CC1(C)Cc2cc3n(c2C1)CCN(Cc1ccc(-c2ccnc4[nH]nc(N[C@@H]5CCNC5)c24)cc1F)C3=O] | 513.62 | 514.5 | 10-{[2-fluoro-4-(3- {[(3R)-pyrrolidin-3-yl]amino}- 1H-pyrazolo[3,4-b]pyridin- 4-yl)phenyl]methyl}-4,4-dimethyl- 1,10-diazatricyclo[6.4.0.0²,6] dodeca-2(6),7-dien-9-one | Method D |
| 53 | 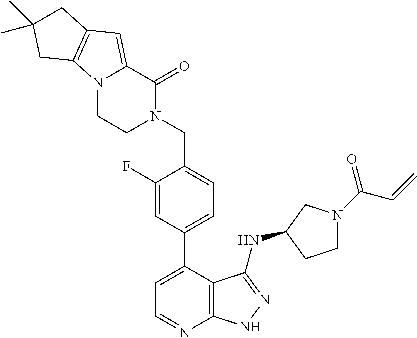 [SMILES: C=CC(=O)N1CC[C@@H](Nc2n[nH]c3nccc(-c4ccc(CN5CCn6c(cc7c6CC(C)(C)C7)C5=O)c(F)c4)c23)C1] | 567.67 | 568.2 | 10-{[2-fluoro-4-(3-{[(3R)-1-(prop- 2-enoyl)pyrrolidin- 3-yl]amino}-1H- pyrazolo[3,4-b]pyridin-4- yl)phenyl]methyl}-4,4- dimethyl-1,10- diazatricyclo[6.4.0.0²,6]dodeca- 2(6),7-dien-9-one | Method E |
| 54 | 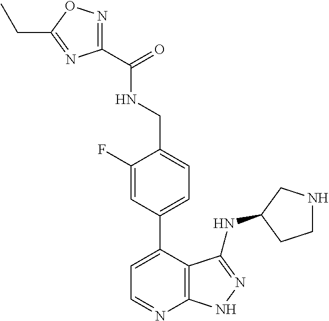 [SMILES: CCc1nc(C(=O)NCc2ccc(-c3ccnc4[nH]nc(N[C@@H]5CCNC5)c34)cc2F)no1] | 450.48 | 451.1 | 5-ethyl-N-{[2-fluoro-4-(3-{[(3R)- pyrrolidin-3-yl]amino}-1H- pyrazolo[3,4-b]pyridin-4- yl)phenyl]methyl}-1,2,4- oxadiazole-3-carboxamide | Method D |
| 55 | 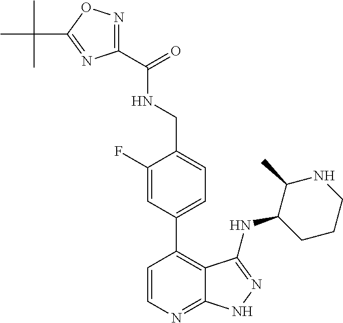 [SMILES: C[C@H]1NCCC[C@H]1Nc1n[nH]c2nccc(-c3ccc(CNC(=O)c4noc(C(C)(C)C)n4)c(F)c3)c12] | 506.59 | 507 | 5-tert-butyl-N-{[2-fluoro-4-(3- {[(2R,3R)-2-methylpiperidin-3- yl]amino}-1H- pyrazolo[3,4-b]pyridin-4-yl) phenyl]methyl}-1,2,4-oxadiazole- 3-carboxamide | Method D |
| 56 | 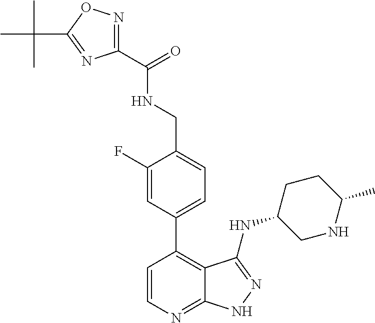 [SMILES: C[C@H]1CC[C@@H](Nc2n[nH]c3nccc(-c4ccc(CNC(=O)c5noc(C(C)(C)C)n5)c(F)c4)c23)CN1] | 506.59 | 507 | 5-tert-butyl-N-{[2-fluoro-4-(3- {[(3R,6S)-6-methylpiperidin-3- yl]amino}-1H- pyrazolo[3,4-b]pyridin-4-yl) phenyl]methyl}-1,2,4-oxadiazole- 3-carboxamide | Method D |
| 57 | 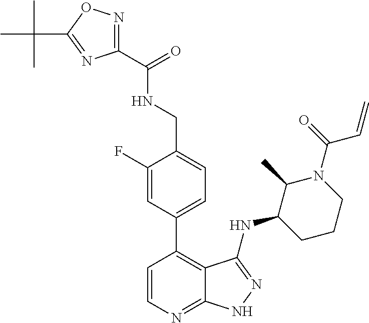 [SMILES: C=CC(=O)N1CCC[C@@H](Nc2n[nH]c3nccc(-c4ccc(CNC(=O)c5noc(C(C)(C)C)n5)c(F)c4)c23)[C@H]1C] | 560.63 | 561.2 | 5-tert-butyl-N-{[2-fluoro-4-(3- {[(2R,3R)-2-methyl-1-(prop-2- enoyl)piperidin-3-yl]amino}-1H- pyrazolo[3,4-b]pyridin-4- yl)phenyl]methyl}-1,2,4- oxadiazole-3-carboxamide | Method E |
| 58 | 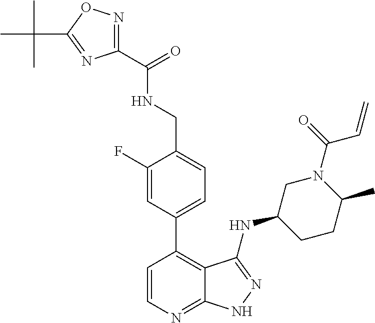 [SMILES: C=CC(=O)N1C[C@H](Nc2n[nH]c3nccc(-c4ccc(CNC(=O)c5noc(C(C)(C)C)n5)c(F)c4)c23)CC[C@@H]1C] | 560.63 | 561.2 | 5-tert-butyl-N-{[2-fluoro-4-(3- {[(3R,6S)-6-methyl-1-(prop-2- enoyl)piperidin-3-yl]amino}-1H- pyrazolo[3,4-b]pyridin-4- yl)phenyl]methyl}-1,2,4- oxadiazole-3-carboxamide | Method E |
| 59 | 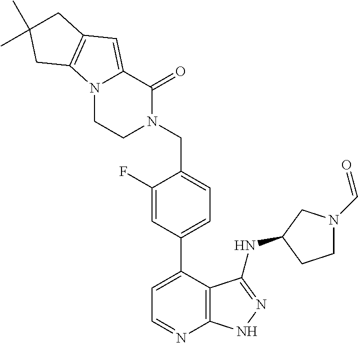 [SMILES: CC1(C)Cc2cc3n(c2C1)CCN(Cc1ccc(-c2ccnc4[nH]nc(N[C@@H]5CCN(C=O)C5)c24)cc1F)C3=O] | 541.63 | 542.2 | (3R)-3-({4-[4- ({4,4-dimethyl-9-oxo- 1,10-diazatricyclo [6..0.0²,6]dodeca- 2(6),7-dien-10-yl} methyl)-3- fluorophenyl]-1H- pyrazolo[3,4- b]pyridin-3-yl}amino) pyrrolidine- 1-carbaldehyde | Method E* (formed as by- product from starting material contami- nated with formic acid) |
| 60 | 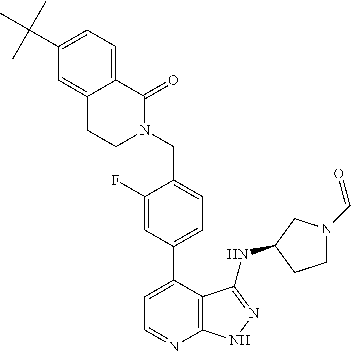 [SMILES: CC(C)(C)c1ccc2c(c1)CCN(Cc1ccc(-c3ccnc4[nH]nc(N[C@@H]5CCN(C=O)C5)c34)cc1F)C2=O] | 540.64 | 541.2 | (3R)-3-[(4-{4-[(6- tert-butyl-1-oxo- 1,2,3,4-tetrahydroisoquinolin-2- yl)methyl]-3-fluorophenyl}-1H- pyrazolo[3,4-b]pyridin-3- yl)amino]pyrrolidine-1- carbaldehyde | Method E* (formed as by- product from starting material contami- nated with formic acid) |
| 61 | 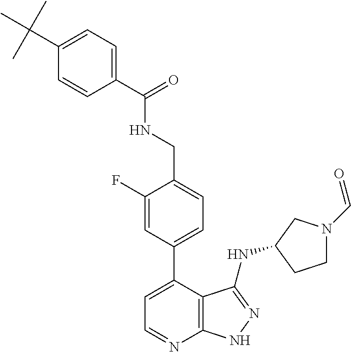 [SMILES: CC(C)(C)c1ccc(C(=O)NCc2ccc(-c3ccnc4[nH]nc(N[C@H]5CCN(C=O)C5)c34)cc2F)cc1] | 514.61 | 515.2 | 4-tert-butyl-N-{[2-fluoro- 4-(3-{[(3S)-1- formylpyrrolidin-3- yl]amino}-1H- pyrazolo[3,4-b]pyridin-4- yl)phenyl]methyl}benzamide | Method E* (formed as by- product from starting material contami- nated with formic acid) |
| 62 | 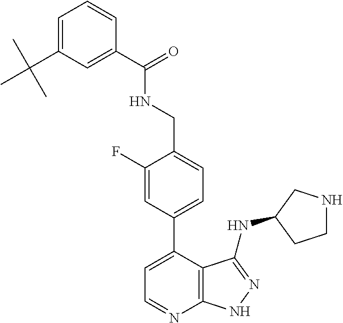 [SMILES: CC(C)(C)c1cccc(C(=O)NCc2ccc(-c3ccnc4[nH]nc(N[C@@H]5CCNC5)c34)cc2F)c1] | 486.60 | 487.2 | 3-tert-butyl- N-{[2-fluoro-4-(3-{[(3R)- pyrrolidin-3-yl]amino}-1H- pyrazolo[3,4-b]pyridin-4- yl)phenyl]methyl}benzamide | Method D |
| 63 | 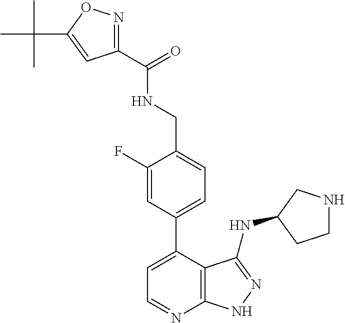 [SMILES: CC(C)(C)c1cc(C(=O)NCc2ccc(-c3ccnc4[nH]nc(N[C@@H]5CCNC5)c34)cc2F)no1] | 477.54 | 478.2 | 5-tert-butyl-N-{[2- fluoro-4-(3-{[(3R)- pyrrolidin-3-yl]amino}-1H- pyrazolo[3,4-b]pyridin-4- yl)phenyl]methyl}- 1,2-oxazole-3- carboxamide | Method D |
| 64 | 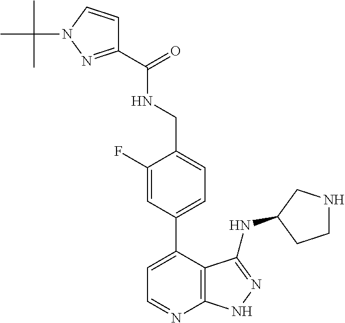 [SMILES: CC(C)(C)n1ccc(C(=O)NCc2ccc(-c3ccnc4[nH]nc(N[C@@H]5CCNC5)c34)cc2F)n1] | 476.56 | 477.2 | 1-tert-butyl-N-{[2- fluoro-4-(3-{[(3R)- pyrrolidin-3-yl]amino}-1H- pyrazolo[3,4-b]pyridin-4- yl)phenyl]methyl}- 1H-pyrazole-3- carboxamide | Method D |
| 65 | 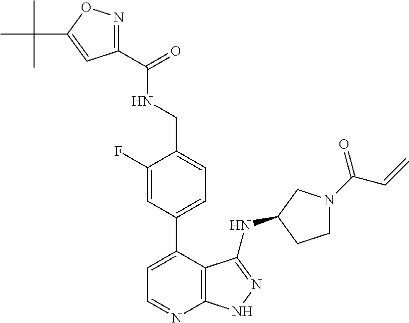 [SMILES: C=CC(=O)N1CC[C@@H](Nc2n[nH]c3nccc(-c4ccc(CNC(=O)c5cc(C(C)(C)C)on5)c(F)c4)c23)C1] | 531.59 | 532.2 | 5-tert-butyl-N-{[2- fluoro-4-(3-{[(3R)- 1-(prop-2-enoyl)pyrrolidin-3-yl] amino}-1H-pyrazolo [3,4-b]pyridin- 4-yl)phenyl]methyl}- 1,2-oxazole-3- carboxamide | Method E |
| 66 | 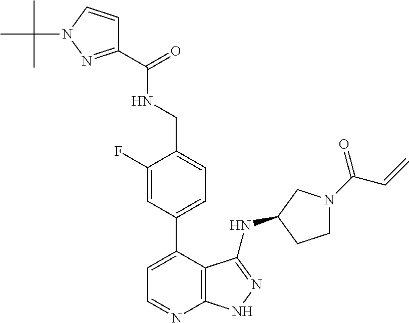 [SMILES: C=CC(=O)N1CC[C@@H](Nc2n[nH]c3nccc(-c4ccc(CNC(=O)c5ccn(C(C)(C)C)n5)c(F)c4)c23)C1] | 530.61 | 531.2 | 1-tert-butyl-N-{[2- fluoro-4-(3-{[(3R)- 1-(prop-2-enoyl)pyrrolidin-3- yl]amino}-1H- pyrazolo[3,4-b]pyridin- 4-yl)phenyl]methyl}- 1H-pyrazole- 3-carboxamide | Method E |
| 67 | 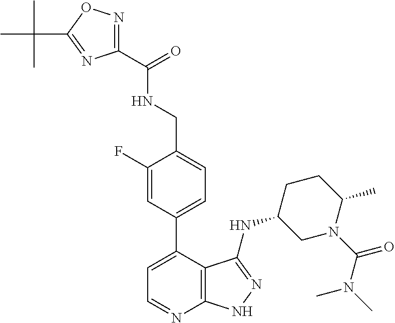 [SMILES: C[C@H]1CC[C@@H](Nc2n[nH]c3nccc(-c4ccc(CNC(=O)c5noc(C(C)(C)C)n5)c(F)c4)c23)CN1C(=O)N(C)C] | 577.67 | 578.2 | (2S,5R)-5-{[4-(4-{[(5- tert-butyl-1,2,4-oxadiazol- 3-yl)formamido]methyl}-3- fluorophenyl)-1H-pyrazolo[3,4- b]pyridin-3-yl]amino}-N,N,2- trimethylpiperidine-1-carboxamide | Method E |
| 68 | 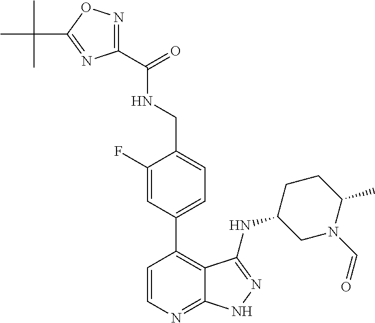 [SMILES: C[C@H]1CC[C@@H](Nc2n[nH]c3nccc(-c4ccc(CNC(=O)c5noc(C(C)(C)C)n5)c(F)c4)c23)CN1C=O] | 534.60 | 535.1 | 5-tert-butyl-N-{[2-fluoro-4-(3- {[(3R,6S)-1-formyl-6- methylpiperidin- 3-yl]amino}-1H-pyrazolo[3,4- b]pyridin-4-yl) phenyl]methyl}-1,2,4- oxadiazole-3-carboxamide | Method E* (formed as by- product from starting material contam- inated with formic acid) |
| 69 | 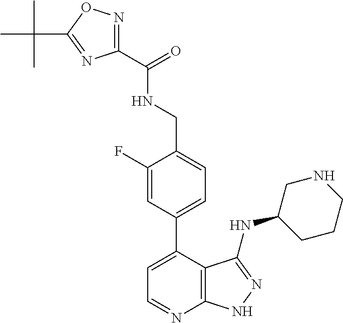 [SMILES: CC(C)(C)c1nc(C(=O)NCc2ccc(-c3ccnc4[nH]nc(N[C@@H]5CCCNC5)c34)cc2F)no1] | 492.56 | 493.5 | 5-tert-butyl-N-{[2-fluoro- 4-(3-{[(3R)- piperidin-3-yl]amino}-1H- pyrazolo[3,4-b]pyridin-4- yl)phenyl]methyl}-1,2,4- oxadiazole-3-carboxamide | Method D |
| 70 | 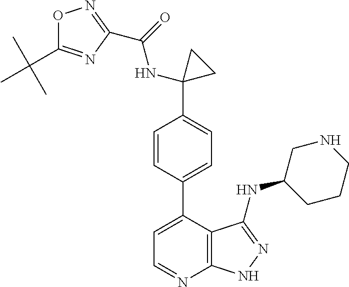 [SMILES: CC(C)(C)c1nc(C(=O)NC2(c3ccc(-c4ccnc5[nH]nc(N[C@@H]6CCCNC6)c45)cc3)CC2)no1] | 500.61 | 501.6 | 5-tert-butyl-N-{1-[4-(3-{[(3R)- piperidin-3-yl]amino}-1H- pyrazolo[3,4-b]pyridin-4- yl)phenyl]cyclopropyl}-1,2,4- oxaidazole-3-carboxamide | Method D |
| 71 | 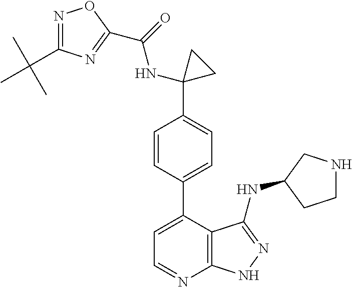 [SMILES: CC(C)(C)c1noc(C(=O)NC2(c3ccc(-c4ccnc5[nH]nc(N[C@@H]6CCNC6)c45)cc3)CC2)n1] | 486.58 | 487.5 | 3-tert-butyl-N-{1-[4-(3-{[(3R)- pyrrolidin-3-yl]amino}-1H- pyrazolo[3,4-b]pyridin-4- yl)phenyl]cyclopropyl}-1,2,4- oxadiazole-5-carboxamide | Method D |
| 72 | 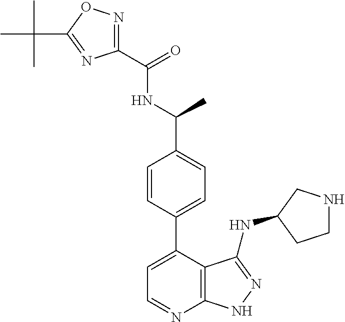 [SMILES: C[C@H](NC(=O)c1noc(C(C)(C)C)n1)c1ccc(-c2ccnc3[nH]nc(N[C@@H]4CCNC4)c23)cc1] | 474.57 | 475.8 | 5-tert-butyl-N-[(1S)-1- [4-(3-{[(3R)- pyrrolidin-3-yl]amino}-1H- pyrazolo[3,4-b]pyridin-4- yl)phenyl]ethyl]-1,2,4-oxadiazole- 3-carboxamide | Method D |
| 73 | 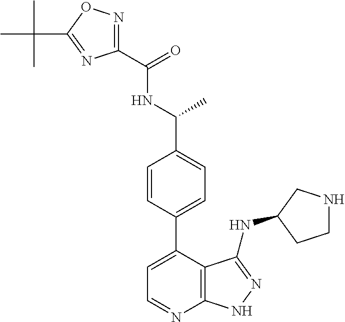 [SMILES: C[C@@H](NC(=O)c1noc(C(C)(C)C)n1)c1ccc(-c2ccnc3[nH]nc(N[C@@H]4CCNC4)c23)cc1] | 474.57 | 475.5 | 5-tert-butyl-N-[(1R)- 1-[4-(3-{[(3R)- pyrrolidin-3-yl]amino}-1H- pyrazolo[3,4-b]pyridin-4- yl)phenyl]ethyl]-1,2,4-oxadiazole- 3-carboxamide | Method D |
| 74 | 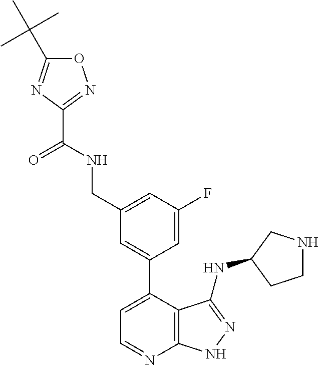 [SMILES: CC(C)(C)c1nc(C(=O)NCc2cc(F)cc(-c3ccnc4[nH]nc(N[C@@H]5CCNC5)c34)c2)no1] | 478.53 | 479.5 | 5-tert-butyl-N-{[3-fluoro- 5-(3-{[(3R)- pyrrolidin-3-yl]amino}-1H- pyrazolo[3,4-b]pyridin-4- yl)phenyl]methyl}-1,2,4- oxadiazole-3-carboxamide | Method D |
| 75 | 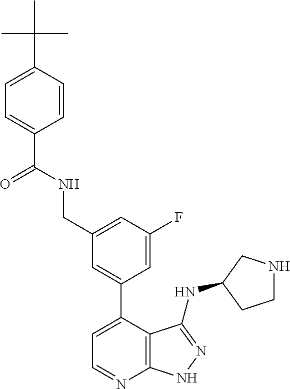 [SMILES: CC(C)(C)c1ccc(C(=O)NCc2cc(F)cc(-c3ccnc4[nH]nc(N[C@@H]5CCNC5)c34)c2)cc1] | 486.60 | 487.5 | 4-tert-butyl-N-{[3-fluoro- 5-(3-{[(3R)- pyrrolidin-3-yl]amino}-1H- pyrazolo[3,4-b]pyridin-4- yl)phenyl]methyl}benzamide | Method D |
| 76 | 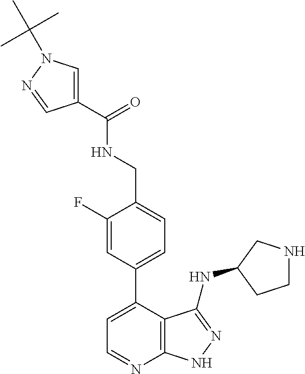 [SMILES: CC(C)(C)n1cc(C(=O)NCc2ccc(-c3ccnc4[nH]nc(N[C@@H]5CCNC5)c34)cc2F)cn1] | 476.56 | 477.2 | 1-tert-butyl-N-{[2-fluoro- 4-(3-{[(3R)- pyrrolidin-3-yl]amino}-1H- pyrazolo[3,4-b]pyridin-4- yl)phenyl]methyl}-1H-pyrazole-4- carboxamide | Method D |
| 77 | 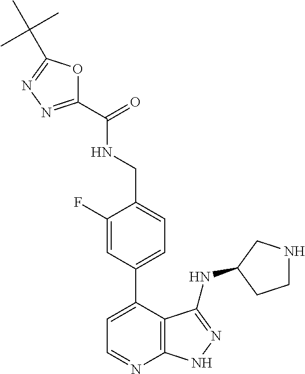 [SMILES: CC(C)(C)c1nnc(C(=O)NCc2ccc(-c3ccnc4[nH]nc(N[C@@H]5CCNC5)c34)cc2F)o1] | 478.53 | 479.2 | 5-tert-butyl-N-{[2-fluoro- 4-(3-{[(3R)- pyrrolidin-3-yl]amino}-1H- pyrazolo[3,4-b]pyridin-4- yl)phenyl]methyl}-1,3,4- oxadiazole-2-carboxamide | Method D |
| 78 | 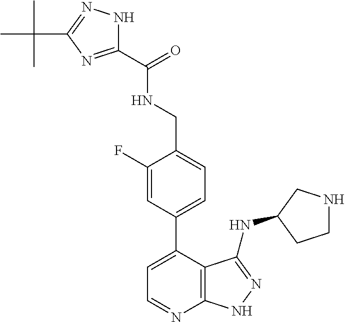 [SMILES: CC(C)(C)c1n[nH]c(C(=O)NCc2ccc(-c3ccnc4[nH]nc(N[C@@H]5CCNC5)c34)cc2F)n1] | 477.55 | 478.2 | 3-tert-butyl-N-{[2-fluoro- 4-(3-{[(3R)- pyrrolidin-3-yl]amino}-1H- pyrazolo[3,4-b]pyridin-4- yl)phenyl]methyl}-1H-1,2,4- triazole-5-carboxamide | Method D |
| 79 | 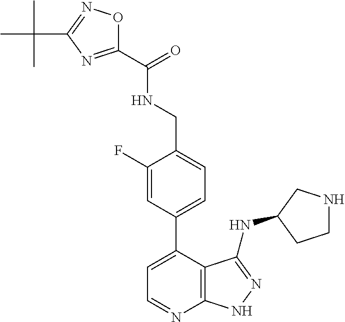 [SMILES: CC(C)(C)c1noc(C(=O)NCc2ccc(-c3ccnc4[nH]nc(N[C@@H]5CCNC5)c34)cc2F)n1] | 478.53 | 479.2 | 3-tert-butyl-N-{[2-fluoro- 4-(3-{[(3R)- pyrrolidin-3-yl]amino}-1H- pyrazolo[3,4-b]pyridin-4- yl)phenyl]methyl}-1,2,4- oxadiazole-5-carboxamide | Method D |
| 80 | 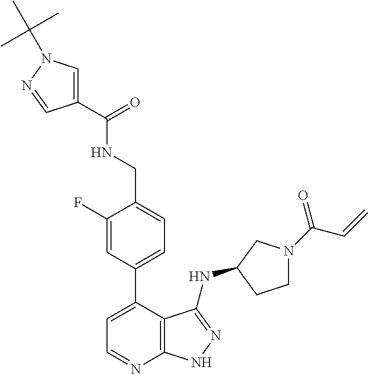 [SMILES: C=CC(=O)N1CC[C@@H](Nc2n[nH]c3nccc(-c4ccc(CNC(=O)c5cnn(C(C)(C)C)c5)c(F)c4)c23)C1] | 530.61 | 531.2 | 1-tert-butyl-N-{[2-fluoro- 4-(3-{[(3R)- 1-(prop-2-enoyl)pyrrolidin-3- yl]amino}-1H- pyrazolo[3,4-b]pyridin- 4-yl)phenyl]methyl}-1H-pyrazole- 4-carboxamide | Method E |
| 81 | 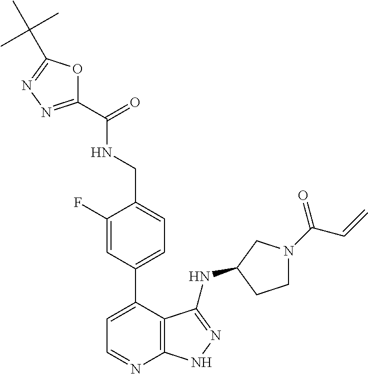 [SMILES: C=CC(=O)N1CC[C@@H](Nc2n[nH]c3nccc(-c4ccc(CNC(=O)c5nnc(C(C)(C)C)o5)c(F)c4)c23)C1] | 532.58 | 533.1 | 5-tert-butyl-N-{[2-fluoro- 4-(3-{[(3R)- 1-(prop-2-enoyl)pyrrolidin-3- yl]amino}-1H- pyrazolo[3,4-b]pyridin- 4-yl)phenyl]methyl}-1,3,4- oxadiazole-2-carboxamide | Method E |
| 82 | 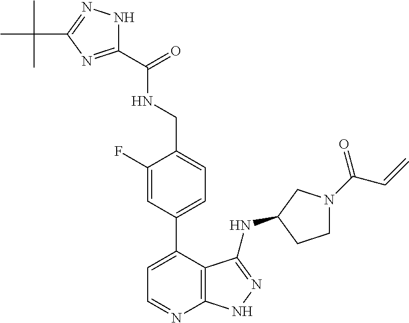 [SMILES: C=CC(=O)N1CC[C@@H](Nc2n[nH]c3nccc(-c4ccc(CNC(=O)c5nc(C(C)(C)C)n[nH]5)c(F)c4)c23)C1] | 531.60 | 532.1 | 3-tert-butyl-N-{[2- fluoro-4-(3-{[(3R)- 1-(prop-2-enoyl)pyrrolidin-3- yl]amino}-1H- pyrazolo[3,4-b]pyridin- 4-yl)phenyl]methyl}-1H- 1,2,4-triazole- 5-carboxamide | Method E |
| 83 | 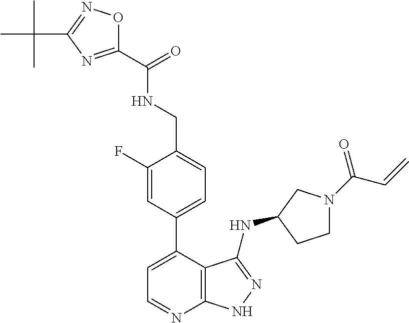 [SMILES: C=CC(=O)N1CC[C@@H](Nc2n[nH]c3nccc(-c4ccc(CNC(=O)c5nc(C(C)(C)C)no5)c(F)c4)c23)C1] | 532.58 | 533.1 | 3-tert-butyl-N-{[2- fluoro-4-(3-{[(3R)- 1-(prop-2-enoyl)pyrrolidin-3- yl]amino}-1H- pyrazolo[3,4-b]pyridin- 4-yl)phenyl]methyl}-1,2,4- oxadiazole-5-carboxamide | Method E |
| 84 | 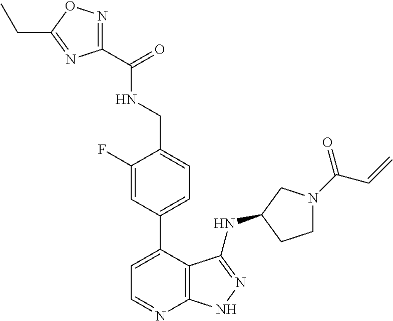 [SMILES: C=CC(=O)N1CC[C@@H](Nc2n[nH]c3nccc(-c4ccc(CNC(=O)c5noc(CC)n5)c(F)c4)c23)C1] | 504.53 | 505.1 | 5-ethyl-N-{[2-fluoro-4-(3-{[(3R)- 1-(prop-2-enoyl)pyrrolidin- 3-yl]amino}- 1H-pyrazolo[3,4-b]pyridin-4- yl)phenyl]methyl}-1,2,4- oxadiazole-3-carboxamide | Method E |
| 85 | 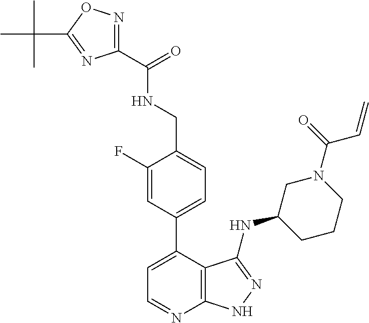 [SMILES: C=CC(=O)N1CCC[C@@H](Nc2n[nH]c3nccc(-c4ccc(CNC(=O)c5noc(C(C)(C)C)n5)c(F)c4)c23)C1] | 546.61 | 547.2 | 5-tert-butyl-N-{[2-fluoro- 4-(3-{[(3R)-1-(prop-2- enoyl)piperidin-3-yl]amino}- 1H-pyrazolo[3,4-b]pyridin-4- yl)phenyl]methyl}-1,2,4- oxadiazole-3-carboxamide | Method E |
| 86 | 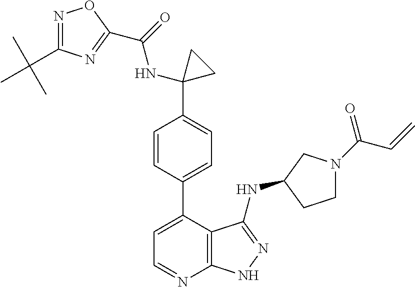 [SMILES: C=CC(=O)N1CC[C@@H](Nc2n[nH]c3nccc(-c4ccc(C5(NC(=O)c6nc(C(C)(C)C)no6)CC5)cc4)c23)C1] | 540.63 | 541.2 | 3-tert-butyl-N-{1-[4- (3-{[(3R)-1-(prop- 2-enoyl)pyrrolidin-3-yl]amino}- 1H-pyrazolo[3,4-b]pyridin-4- yl)phenyl]cyclopropyl}-1,2,4- oxadiazole-5-carboxamide | Method E |
| 87 | 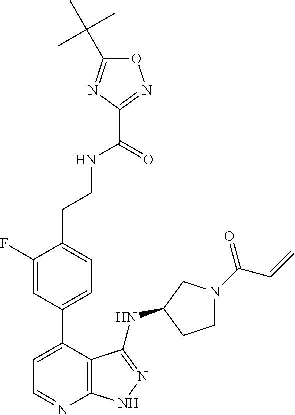 [SMILES: C=CC(=O)N1CC[C@@H](Nc2n[nH]c3nccc(-c4ccc(CCNC(=O)c5noc(C(C)(C)C)n5)c(F)c4)c23)C1] | 546.61 | 547.2 | 5-tert-butyl-N-{2-[2-fluoro-4-(3- {[(3R)-1-(prop-2-enoyl) pyrrolidin-3-yl]amino}- 1H-pyrazolo[3,4-b]pyridin- 4-yl)phenyl]ethyl}-1,2,4- oxadiazole-3-carboxamide | Method E |
| 88 | 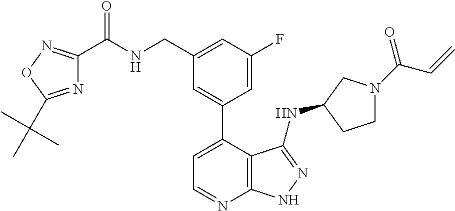 [SMILES: C=CC(=O)N1CC[C@@H](Nc2n[nH]c3nccc(-c4cc(F)cc(CNC(=O)c5noc(C(C)(C)C)n5)c4)c23)C1] | 532.58 | 533.1 | 5-tert-butyl-N-{[3-fluoro- 5-(3-{[(3R)- 1-(prop-2-enoyl)pyrrolidin-3- yl]amino}-1H- pyrazolo[3,4-b]pyridin- 4-yl)phenyl]methyl}-1,2,4- oxadiazole-3-carboxamide | Method E |
| 89 | 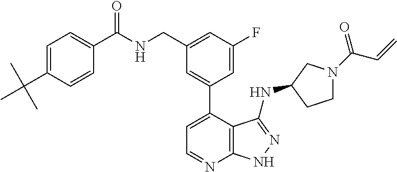 [SMILES: C=CC(=O)N1CC[C@@H](Nc2n[nH]c3nccc(-c4cc(F)cc(CNC(=O)c5ccc(C(C)(C)C)cc5)c4)c23)C1] | 540.64 | 541.2 | 4-tert-butyl-N-{[3- fluoro-5-(3-{[(3R)- 1-(prop-2-enoyl)pyrrolidin-3-yl] amino}-1H- pyrazolo[3,4-b]pyridin- 4-yl)phenyl]methyl}benzamide | Method E |
| 90 | 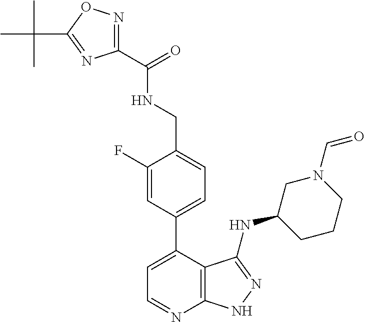 [SMILES: CC(C)(C)c1nc(C(=O)NCc2ccc(-c3ccnc4[nH]nc(N[C@@H]5CCCN(C=O)C5)c34)cc2F)no1] | 520.57 | 521.2 | 5-tert-butyl-N-{[2-fluoro- 4-(3-{[(3R)- 1-formylpiperidin-3-yl]amino}- 1H-pyrazolo[3,4-b]pyridin-4- yl)phenyl]methyl}-1,2,4- oxadiazole-3-carboxamide | Method E* (formed as by- product from starting material contami- nated with formic acid) |
| 91 | 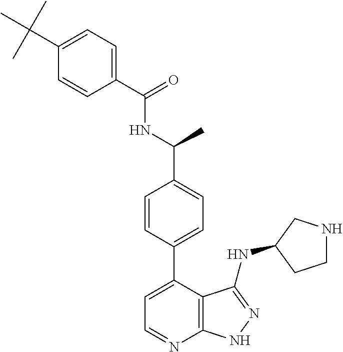 [SMILES: C[C@H](NC(=O)c1ccc(C(C)(C)C)cc1)c1ccc(-c2ccnc3[nH]nc(N[C@@H]4CCNC4)c23)cc1] | 482.63 | 483.5 | 4-tert-butyl-N-[(1S)- 1-[4-(3-{[(3R)- pyrrolidin-3-yl]amino}-1H- pyrazolo[3,4-b]pyridin-4- yl)phenyl]ethyl]benzamide | Method D |
| 92 | 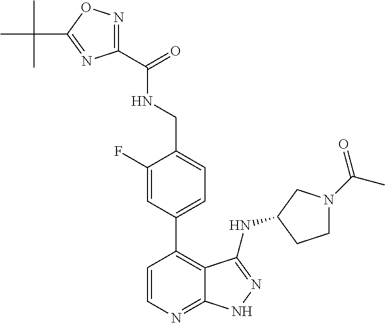 [SMILES: CC(=O)N1CC[C@H](Nc2n[nH]c3nccc(-c4ccc(CNC(=O)c5noc(C(C)(C)C)n5)c(F)c4)c23)C1] | 520.57 | 521.2 | N-{[4-(3-{[(3S)-1- acetylpyrrolidin-3-yl]amino}- 1H-pyrazolo[3,4-b]pyridin- 4-yl)-2-fluorophenyl]methyl}- 5-tert-butyl-1,2,4- oxadiazole-3-carboxamide | Method E |
| 93 | 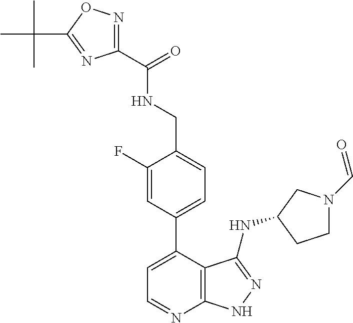 [SMILES: CC(C)(C)c1nc(C(=O)NCc2ccc(-c3ccnc4[nH]nc(N[C@H]5CCN(C=O)C5)c34)cc2F)no1] | 506.54 | 507.1 | 5-tert-butyl- N-{[2-fluoro-4-(3-{[(3S)- 1-formylpyrrolidin-3-yl]amino}- 1H-pyrazolo[3,4-b]pyridin-4- yl)phenyl]methyl}-1,2,4- oxadiazole-3-carboxamide | Method E* (formed as by- product from starting material contami- nated with formic acid) |
| 94 | 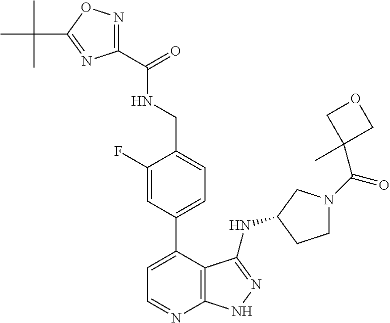 [SMILES: CC1(C(=O)N2CC[C@H](Nc3n[nH]c4nccc(-c5ccc(CNC(=O)c6noc(C(C)(C)C)n6)c(F)c5)c34)C2)COC1] | 576.63 | 577.2 | 5-tert-butyl-N-{[2-fluoro- 4-(3-{[(3S)- 1-(3-methyloxetane-3- carbonyl)pyrrolidin-3-yl]amino}- 1H-pyrazolo[3,4-b]pyridin-4- yl)phenyl]methyl}-1,2,4- oxadiazole-3-carboxamide | Method C |
| 95 | 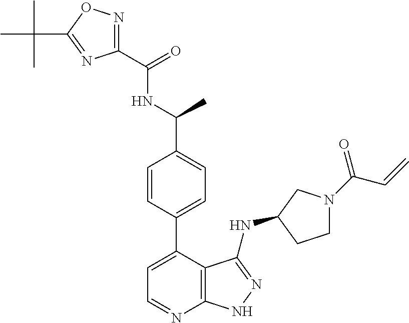 [SMILES: C=CC(=O)N1CC[C@@H](Nc2n[nH]c3nccc(-c4ccc([C@H](C)NC(=O)c5noc(C(C)(C)C)n5)cc4)c23)C1] | 528.62 | 529.3 | 5-tert-butyl- N-[(1S)-1-[4-(3-{[(3R)-1- (prop-2-enoyl)pyrrolidin- 3-yl]amino}- 1H-pyrazolo[3,4-b]pyridin-4- yl)phenyl]ethyl]-1,2,4-oxadiazole- 3-carboxamide | Method E |
| 96 | 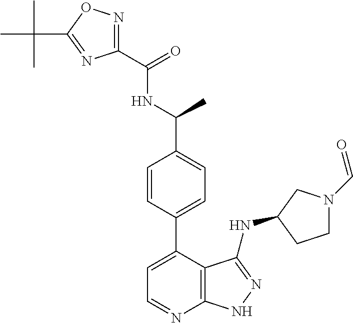 [SMILES: C[C@H](NC(=O)c1noc(C(C)(C)C)n1)c1ccc(-c2ccnc3[nH]nc(N[C@@H]4CCN(C=O)C4)c23)cc1] | 502.58 | 503.2 | 5-tert-butyl-N-[(1S)- 1-[4-(3-{[(3R)-1- formylpyrrolidin-3-yl]amino}-1H- pyrazolo[3,4-b]pyridin-4- yl)phenyl]ethyl]-1,2,4-oxadiazole- 3-carboxamide | Method E* (formed as by- product from starting material contami- nated with formic acid) |
| 97 | 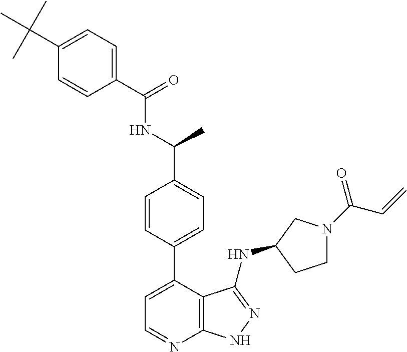 [SMILES: C=CC(=O)N1CC[C@@H](Nc2n[nH]c3nccc(-c4ccc([C@H](C)NC(=O)c5ccc(C(C)(C)C)cc5)cc4)c23)C1] | 536.68 | 537.2 | 4-tert-butyl-N-[(1S)- 1-[4-(3-{[(3R)-1-(prop-2- enoyl)pyrrolidin-3-yl]amino}- 1H-pyrazolo[3,4-b]pyridin-4- yl)phenyl]ethyl]benzamide | Method E |
| 98 | 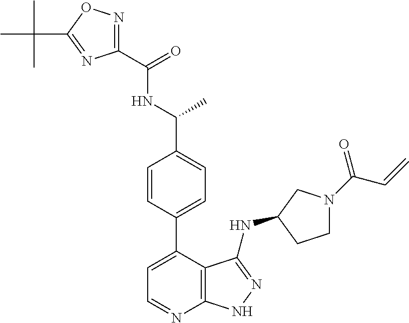 [SMILES: C=CC(=O)N1CC[C@@H](Nc2n[nH]c3nccc(-c4ccc([C@@H](C)NC(=O)c5noc(C(C)(C)C)n5)cc4)c23)C1] | 528.62 | 529.2 | 5-tert-butyl-N-[(1R)- 1-[4-(3-{[(3R)-1-(prop-2- enoyl)pyrrolidin-3-yl]amino}- 1H-pyrazolo[3,4-b]pyridin-4- yl)phenyl]ethyl]-1,2,4-oxadiazole- 3-carboxamide | Method E |
| 99 | 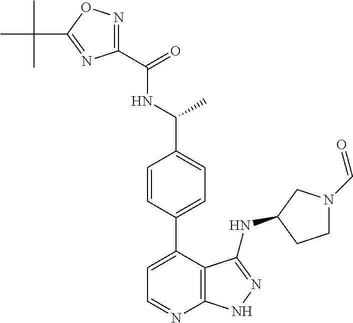 [SMILES: C[C@@H](NC(=O)c1noc(C(C)(C)C)n1)c1ccc(-c2ccnc3[nH]nc(N[C@@H]4CCN(C=O)C4)c23)cc1] | 502.58 | 503.2 | 5-tert-butyl- N-[(1R)-1-[4-(3-{[(3R)-1- formylpyrrolidin-3-yl]amino}-1H- pyrazolo[3,4-b]pyridin-4- yl)phenyl]ethyl]-1,2,4-oxadiazole- 3-carboxamide | Method E* (formed as by- product from starting material contami- nated with formic acid) |
| 100 | 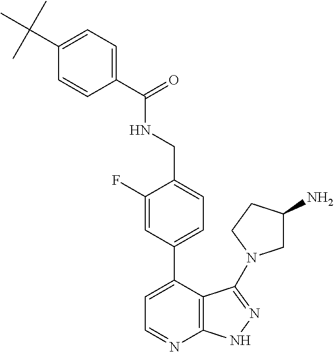 [SMILES: CC(C)(C)c1ccc(C(=O)NCc2ccc(-c3ccnc4[nH]nc(N5CC[C@@H](N)C5)c34)cc2F)cc1] | 486.60 | 487.5 | N-[(4-{3-[(3R)-3- aminopyrrolidin-1-yl]-1H- pyrazolo[3,4-b]pyridin-4-yl}-2- fluorophenyl)methyl]-4-tert- butylbenzamide | Method D |
| 101 | 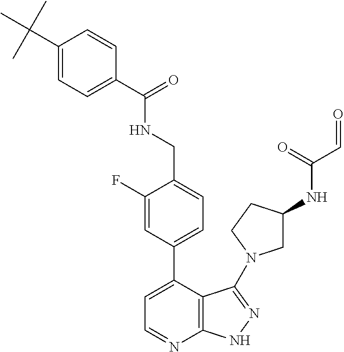 [SMILES: CC(C)(C)c1ccc(C(=O)NCc2ccc(-c3ccnc4[nH]nc(N5CC[C@@H](NC(=O)C=O)C5)c34)cc2F)cc1] | 540.64 | 541.5 | 4-tert-butyl-N-[(2-fluoro- 4-{3-[(3R)-3- (prop-2-enamido)pyrrolidin-1-yl]- 1H-pyrazolo[3,4-b]pyridin-4- yl}phenyl)methyl]benzamide | Method E |
| 102 | 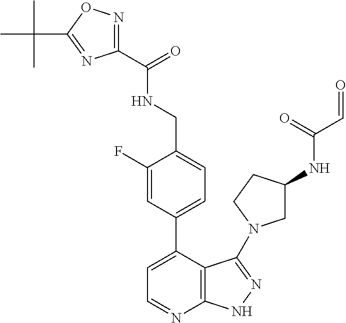 [SMILES: CC(C)(C)c1nc(C(=O)NCc2ccc(-c3ccnc4[nH]nc(N5CC[C@@H](NC(=O)C=O)C5)c34)cc2F)no1] | 532.58 | 533.5 | 5-tert-butyl- N-[(2-fluoro-4-{3-[(3R)-3- (prop-2-enamido)pyrrolidin-1-yl]- 1H-pyrazolo[3,4-b]pyridin-4- yl}phenyl)methyl]-1,2,4- oxadiazole-3-carboxamide | Method E |
| 103 | 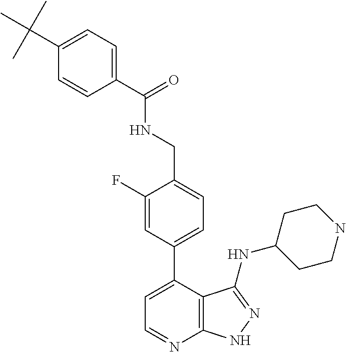 [SMILES: CC(C)(C)c1ccc(C(=O)NCc2ccc(-c3ccnc4[nH]nc(NC5CCNCC5)c34)cc2F)cc1] | 500.62 | 501.5 | 4-tert-butyl-N-[(2-fluoro-4-{3- [(piperidin-4-yl)amino]-1H- pyrazolo[3,4-b]pyridin-4- yl}phenyl)methyl]benzamide | Method D |
| 104 | 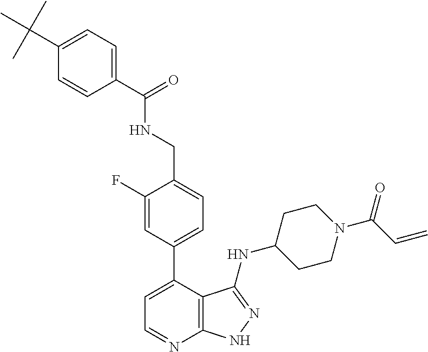 [SMILES: C=CC(=O)N1CCC(Nc2n[nH]c3nccc(-c4ccc(CNC(=O)c5ccc(C(C)(C)C)cc5)c(F)c4)c23)CC1] | 554.67 | 555.5 | 4-tert-butyl-N-{[2-fluoro-4-(3- {[1-(prop-2-enoyl)piperidin- 4-yl]amino}- 1H-pyrazolo[3,4-b]pyridin-4- yl)phenyl]methyl}benzamide | Method E |
| 105 | 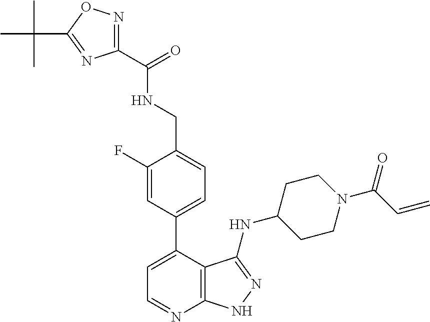 [SMILES: C=CC(=O)N1CCC(Nc2n[nH]c3nccc(-c4ccc(CNC(=O)c5noc(C(C)(C)C)n5)c(F)c4)c23)CC1] | 546.61 | 547.5 | 5-tert-butyl-N-{[2-fluoro- 4-(3-{[1-(prop-2-enoyl) piperidin-4-yl]amino}- 1H-pyrazolo[3,4-b]pyridin-4- yl)phenyl]methyl}-1,2,4- oxadiazole-3-carboxamide | Method E |
| 106 | 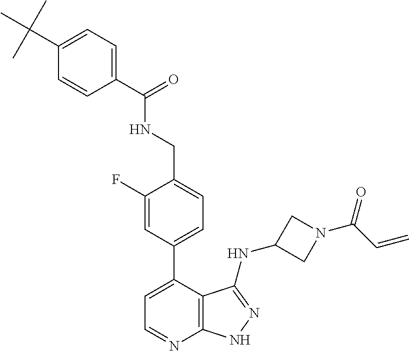 [SMILES: C=CC(=O)N1CC(Nc2n[nH]c3nccc(-c4ccc(CNC(=O)c5ccc(C(C)(C)C)cc5)c(F)c4)c23)C1] | 526.62 | 527.5 | 4-tert-butyl-N-{[2-fluoro- 4-(3-{[1-(prop-2- enoyl)azetidin-3-yl]amino}- 1H-pyrazolo[3,4-b]pyridin-4- yl)phenyl]methyl}benzamide | Method E |
| 107 | 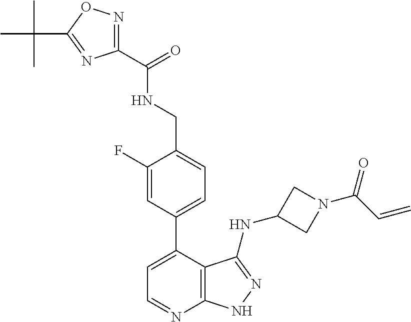 [SMILES: C=CC(=O)N1CC(Nc2n[nH]c3nccc(-c4ccc(CNC(=O)c5noc(C(C)(C)C)n5)c(F)c4)c23)C1] | 518.55 | 519.5 | 5-tert-butyl-N-{[2-fluoro- 4-(3-{[1-(prop-2- enoyl)azetidin-3-yl]amino}- 1H-pyrazolo[3,4-b]pyridin-4- yl)phenyl]methyl}-1,2,4- oxadiazole-3-carboxamide | Method E |
| 108 | 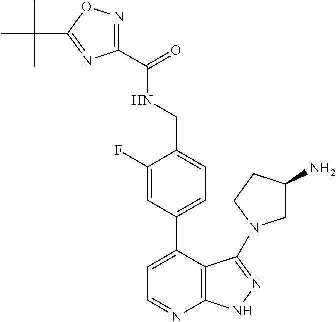 [SMILES: CC(C)(C)c1nc(C(=O)NCc2ccc(-c3ccnc4[nH]nc(N5CC[C@@H](N)C5)c34)cc2F)no1] | 478.53 | 479.9 | N-[(4-{3-[(3R)-3- aminopyrrolidin-1-yl]-1H- pyrazolo[3,4-b]pyridin-4-yl}-2- fluorophenyl)methyl]- 5-tert-butyl- 1,2,4-oxadiazole-3-carboxamide | Method D |
| 109 | 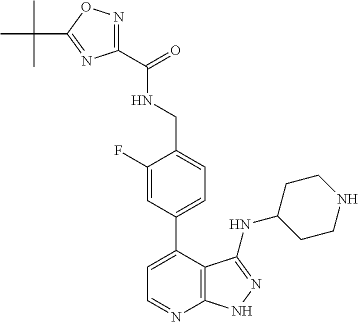 [SMILES: CC(C)(C)c1nc(C(=O)NCc2ccc(-c3ccnc4[nH]nc(NC5CCNCC5)c34)cc2F)no1] | 492.56 | 493.9 | 5-tert-butyl-N-[(2-fluoro-4-{3- [(piperidin-4-yl)amino]-1H- pyrazolo[3,4-b]pyridin-4- yl}phenyl)methyl]-1,2,4- oxadiazole-3-carboxamide | Method D |
| 110 | 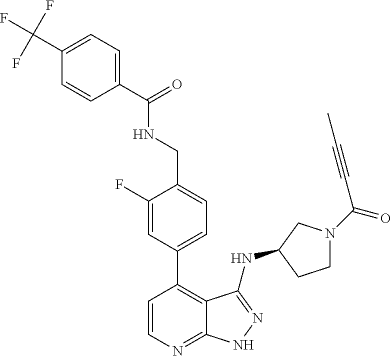 [SMILES: CC#CC(=O)N1CC[C@@H](Nc2n[nH]c3nccc(-c4ccc(CNC(=O)c5ccc(C(F)(F)F)cc5)c(F)c4)c23)C1] | 564.55 | 565.2 | N-{[4-(3-{[(3R)-1-(but-2- ynoyl)pyrrolidin-3-yl]amino}- 1H-pyrazolo[3,4-b]pyridin-4-yl)- 2-fluorophenyl]methyl}-4- (trifluoromethyl)benzamide | Method C |
| 111 | 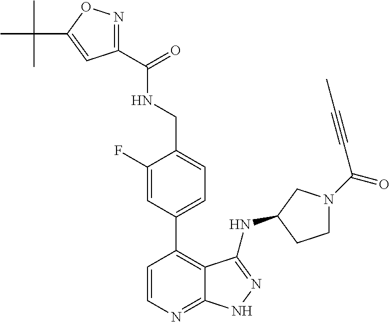 [SMILES: CC#CC(=O)N1CC[C@@H](Nc2n[nH]c3nccc(-c4ccc(CNC(=O)c5cc(C(C)(C)C)on5)c(F)c4)c23)C1] | 543.60 | 544.2 | N-{[4-(3-{[(3R)-1-(but-2- ynoyl)pyrrolidin-3-yl]amino}-1H- pyrazolo[3,4-b]pyridin-4-yl)-2- fluorophenyl]methyl}- 5-tert-butyl-1,2- oxazole-3-carboxamide | Method C |
| 112 | 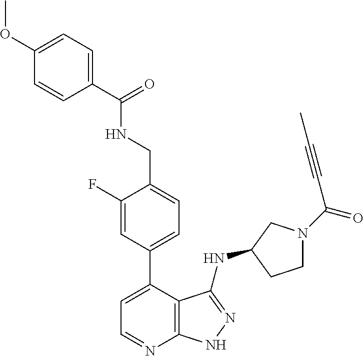 [SMILES: CC#CC(=O)N1CC[C@@H](Nc2n[nH]c3nccc(-c4ccc(CNC(=O)c5ccc(OC)cc5)c(F)c4)c23)C1] | 526.57 | 527.2 | N-{[4-(3-{[(3R)-1-(but-2- ynoyl)pyrrolidin-3-yl]amino}-1H- pyrazolo[3,4-b]pyridin-4-yl)-2- fluorophenyl]methyl}-4- methoxybenzamide | Method C |
| 113 | 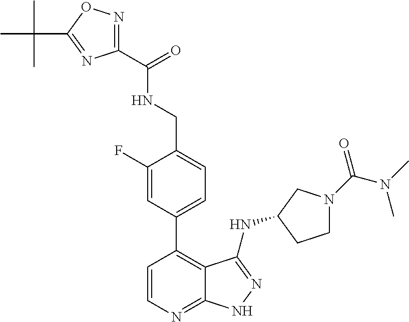 [SMILES: CN(C)C(=O)N1CC[C@H](Nc2n[nH]c3nccc(-c4ccc(CNC(=O)c5noc(C(C)(C)C)n5)c(F)c4)c23)C1] | 549.61 | 550.8 | 5-tert-butyl-N-{[4-(3-{[(3S)-1- (dimethylcarbamoyl)pyrrolidin-3- yl]amino}-1H- pyrazolo[3,4-b]pyridin- 4-yl)-2-fluorophenyl]methyl}- 1,2,4-oxadiazole-3-carboxamide | Method E |
| 114 | 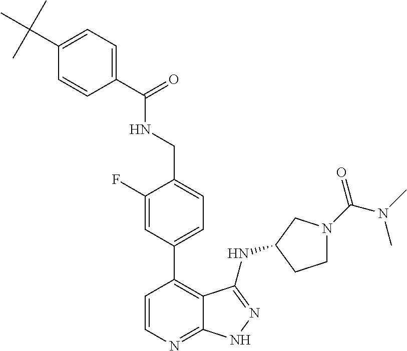 [SMILES: CN(C)C(=O)N1CC[C@H](Nc2n[nH]c3nccc(-c4ccc(CNC(=O)c5ccc(C(C)(C)C)cc5)c(F)c4)c23)C1] | 557.67 | 558.56 | (3S)-3-{[4-(4-{[(4-tert- butylphenyl)formamido] methyl}-3- fluorophenyl)-1H-pyrazolo[3,4- b]pyridin-3-yl]amino}-N,N- dimethylpyrrolidine-1- carboxamide | Method E |
| 115 | 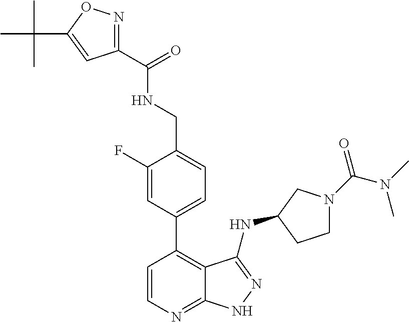 [SMILES: CN(C)C(=O)N1CC[C@@H](Nc2n[nH]c3nccc(-c4ccc(CNC(=O)c5cc(C(C)(C)C)on5)c(F)c4)c23)C1] | 548.62 | 549.8 | 5-tert-butyl-N-{[4-(3-{[(3R)-1- (dimethylcarbamoyl)pyrrolidin-3- yl]amino}-1H- pyrazolo[3,4-b]pyridin- 4-yl)-2-fluorophenyl]methyl}-1,2- oxazole-3-carboxamide | Method E |
| 116 | 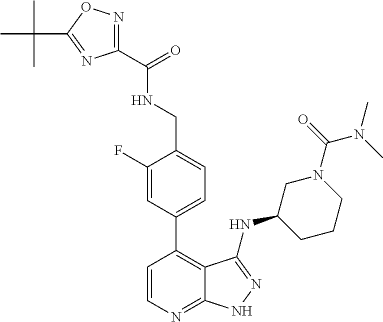 [SMILES: CN(C)C(=O)N1CCC[C@@H](Nc2n[nH]c3nccc(-c4ccc(CNC(=O)c5noc(C(C)(C)C)n5)c(F)c4)c23)C1] | 563.64 | 564.9 | (3R)-3-{[4-(4-{[(5- tert-butyl-1,2,4- oxadiazol-3-yl) formamido]methyl}-3- fluorophenyl)-1H-pyrazolo[3,4- b]pyridin-3-yl]amino}-N,N- dimethylpiperidine-1-carboxamide | Method E |
| 117 | 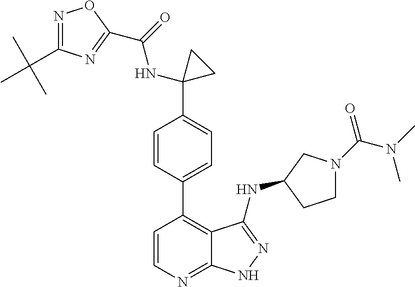 [SMILES: CN(C)C(=O)N1CC[C@@H](Nc2n[nH]c3nccc(-c4ccc(C5(NC(=O)c6nc(C(C)(C)C)no6)CC5)cc4)c23)C1] | 557.66 | 558.8 | 3-tert-butyl-N-{1-[4-(3-{[(3R)-1- (dimethylcarbamoyl)pyrrolidin-3- yl]amino}-1H- pyrazolo[3,4-b]pyridin- 4-yl)phenyl]cyclopropyl}-1,2,4- oxadiazole-5-carboxamide | Method E |
| 118 | 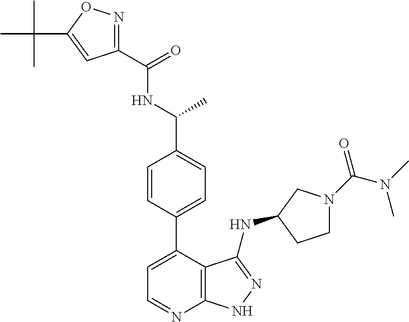 [SMILES: C[C@@H](NC(=O)c1cc(C(C)(C)C)on1)c1ccc(-c2ccnc3[nH]nc(N[C@@H]4CCN(C(=O)N(C)C)C4)c23)cc1] | 545.65 | 547 | 5-tert-butyl-N-[(1R)- 1-[4-(3-{[(3R)-1- (dimethylcarbamoyl)pyrrolidin-3- yl]amino}-1H- pyrazolo[3,4-b]pyridin- 4-yl)phenyl]ethyl]-1,2,4- oxadiazole-3-carboxamide | Method E |
| 119 | 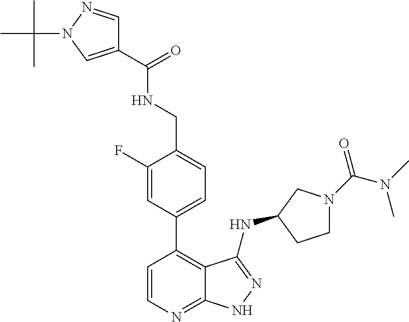 [SMILES: CN(C)C(=O)N1CC[C@@H](Nc2n[nH]c3nccc(-c4ccc(CNC(=O)c5cnn(C(C)(C)C)c5)c(F)c4)c23)C1] | 547.64 | 548.7 | 1-tert-butyl-N-{[4-(3-{[(3R)-1- (dimethylcarbamoyl)pyrrolidin-3- yl]amino}-1H- pyrazolo[3,4-b]pyridin- 4-yl)-2-fluorophenyl]methyl}-1H- pyrazole-4-carboxamide | Method E |
| 120 | 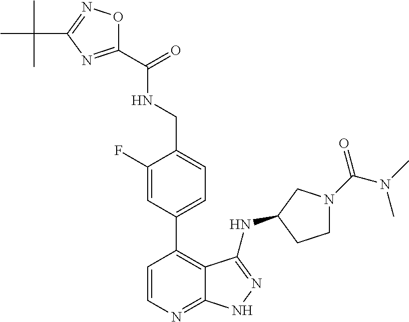 [SMILES: CN(C)C(=O)N1CC[C@@H](Nc2n[nH]c3nccc(-c4ccc(CNC(=O)c5nc(C(C)(C)C)no5)c(F)c4)c23)C1] | 549.61 | 550.9 | 3-tert-butyl-N-{[4-(3-{[(3R)-1- (dimethylcarbmoyl)pyrrolidin-3- yl]amino}-1H- pyrazolo[3,4-b]pyridin- 4-yl)-2-fluorophenyl]methyl}- 1,2,4-oxadiazole-5-carboxamide | Method E |
| 121 | 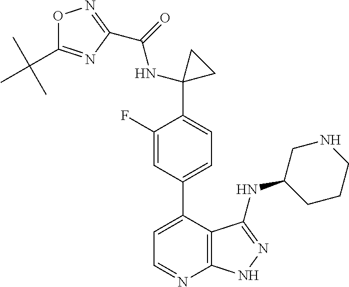 [SMILES: CC(C)(C)c1nc(C(=O)NC2(c3ccc(-c4ccnc5[nH]nc(N[C@@H]6CCCNC6)c45)cc3F)CC2)no1] | 518.60 | 519.7 | 5-tert-butyl-N-{1-[2-fluoro-4-(3- {[(3R)-piperidin-3-yl]amino}-1H- pyrazolo[3,4-b]pyridin-4- yl)phenyl]cyclopropyl}-1,2,4- oxadiazole-3-carboxamide | Method D |
| 122 | 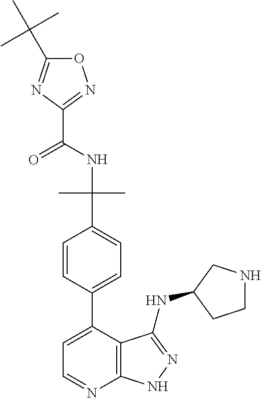 [SMILES: CC(C)(C)c1nc(C(=O)NC(C)(C)c2ccc(-c3ccnc4[nH]nc(N[C@@H]5CCNC5)c34)cc2)no1] | 488.60 | 489.7 | 5-tert-butyl-N-{2-[4-(3-{[(3R)- pyrrolidin-3-yl]amino}-1H- pyraozlo[3,4-b]pyridin-4- yl)phenyl]propan-2-yl}-1,2,4- oxadiazole-3-carboxamide | Method D |
| 123 | 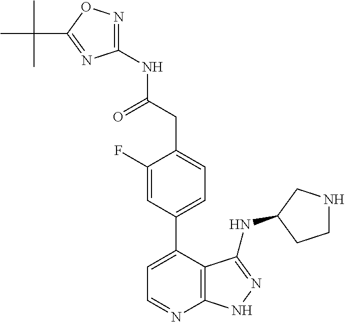 [SMILES: CC(C)(C)c1nc(NC(=O)Cc2ccc(-c3ccnc4[nH]nc(N[C@@H]5CCNC5)c34)cc2F)no1] | 478.53 | 479.7 | N-(5-tert-butyl-1,2,4- oxadiazol-3-yl)-2- [2-fluoro-4-(3-{[(3R)-pyrrolidin- 3-yl]amino}-1H-pyrazolo[3,4-b] pyridin-4-yl)phenyl]acetamide | Method D |
| 124 | 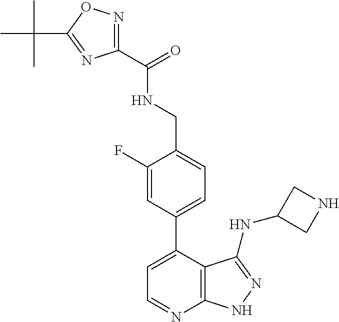 [SMILES: CC(C)(C)c1nc(C(=O)NCc2ccc(-c3ccnc4[nH]nc(NC5CNC5)c34)cc2F)no1] | 464.51 | 465.6 | N-[(4-{3-[(azetidin- 3-yl)amino]-1H- pyrazolo[3,4-b]pyridin-4-yl}-2- fluorophenyl)methyl]-5-tert-butyl- 1,2,4-oxadiazole-3-carboxamide | Method D |
| 125 | 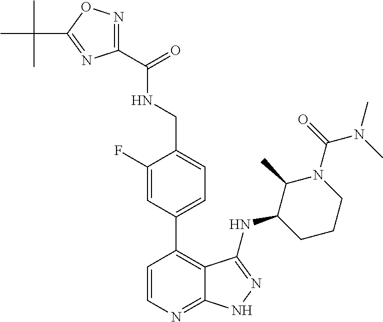 [SMILES: C[C@@H]1[C@H](Nc2n[nH]c3nccc(-c4ccc(CNC(=O)c5noc(C(C)(C)C)n5)c(F)c4)c23)CCCN1C(=O)N(C)C] | 577.67 | 578.2 | (2R,3R)-3-{[4- (4-{[(5-tert-butyl-1,2,4- oxadiaozl-3-yl) formamido]methyl}- 3-fluorophenyl)-1H-pyrazolo[3,4- b]pyridin-3-yl]amino}-N,N,2- trimethylpiperidine-1- carboxamide | Method E |
| 126 | 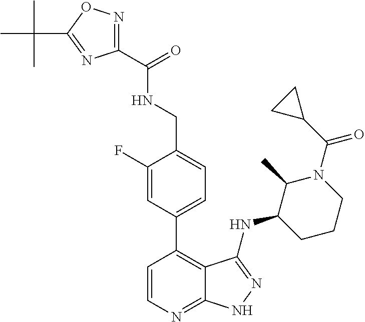 [SMILES: C[C@@H]1[C@H](Nc2n[nH]c3nccc(-c4ccc(CNC(=O)c5noc(C(C)(C)C)n5)c(F)c4)c23)CCCN1C(=O)C1CC1] | 574.66 | 575.1 | 5-tert-butyl-N-{[4-(3-{[(2R,3R)-1- cyclopropanecarbonyl-2- methylpiperidin-3-yl]amino}-1H- pyrazolo[3,4-b]pyridin-4-yl)-2- fluorophenyl]methyl}-1,2,4- oxadiazole-3-carboxamide | Method E |
| 127 | 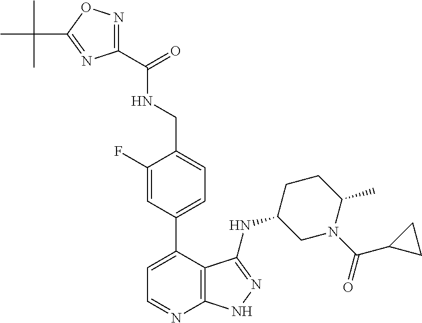 [SMILES: C[C@H]1CC[C@@H](Nc2n[nH]c3nccc(-c4ccc(CNC(=O)c5noc(C(C)(C)C)n5)c(F)c4)c23)CN1C(=O)C1CC1] | 574.66 | 575.2 | 5-tert-butyl-N-{[4-(3-{[(3R,6S)-1- cyclopropanecarbonyl-6- methylpiperidin-3-yl]amino}-1H- pyrazolo[3,4-b]pyridin-4-yl)-2- fluorophenyl]methyl}-1,2,4- oxadiazole-3-carboxamide | Method E |
| 128 | 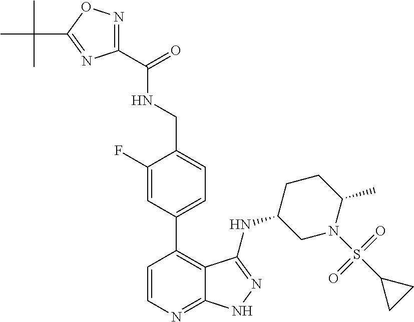 [SMILES: C[C@H]1CC[C@@H](Nc2n[nH]c3nccc(-c4ccc(CNC(=O)c5noc(C(C)(C)C)n5)c(F)c4)c23)CN1S(=O)(=O)C1CC1] | 610.71 | 611.2 | 5-tert-butyl-N-{[4-(3-{[(3R,6S)-1- (cylcopropanesulfonyl)-6- methylpiperidin-3-yl]amino}-1H- pyrazolo[3,4-b]pyridin-4-yl)-2- fluorophenyl]methyl}-1,2,4- oxadiazole-3-carboxamide | Method E |
| 129 | 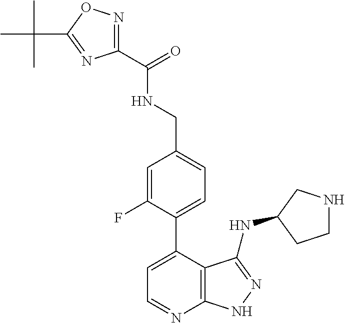 [SMILES: CC(C)(C)c1nc(C(=O)NCc2ccc(-c3ccnc4[nH]nc(N[C@@H]5CCNC5)c34)c(F)c2)no1] | 478.53 | 479.4 | 5-tert-butyl-N-{[3- fluoro-4-(3-{[(3R)- pyrrolidin-3-yl]amino}-1H- pyrazolo[3,4-b]pyridin-4- yl)phenyl]methyl}-1,2,4- oxadiazole-3-carboxamide | Method D |
| 130 | 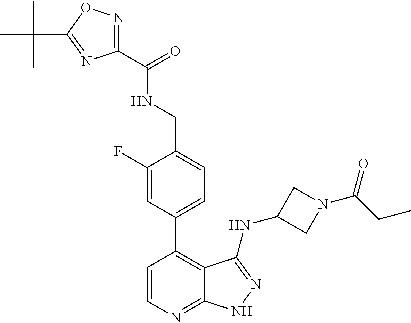 [SMILES: CCC(=O)N1CC(Nc2n[nH]c3nccc(-c4ccc(CNC(=O)c5noc(C(C)(C)C)n5)c(F)c4)c23)C1] | 520.57 | 521.4 | 5-tert-butyl-N-[(2-fluoro-4-{3-[(1- propanoylazetidin-3-yl)amino]-1H- pyrazolo[3,4-b]pyridin-4- yl}phenyl)methyl]-1,2,4- oxadiazole-3-carboxamide | Method C |
| 131 | 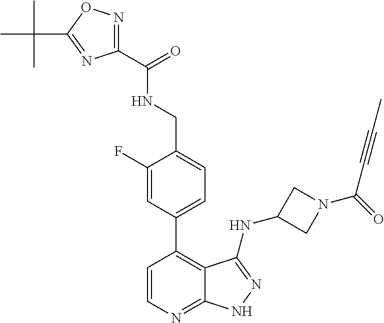 [SMILES: CC#CC(=O)N1CC(Nc2n[nH]c3nccc(-c4ccc(CNC(=O)c5noc(C(C)(C)C)n5)c(F)c4)c23)C1] | 530.56 | 531.4 | N-{[4-(3-{[1-(but-2- ynoyl)azetidin-3- yl]amino}-1H- pyrazolo[3,4-b]pyridin- 4-yl)-2-fluorophenyl]methyl}- 5-tert-butyl-1,2,4-oxadiazole- 3-carboxamide | Method C |
| 132 | 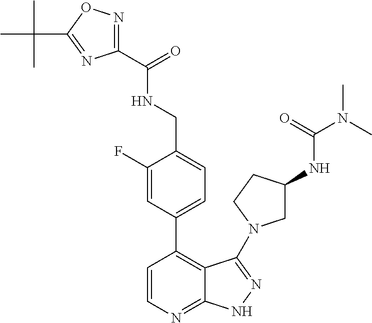 [SMILES: CN(C)C(=O)N[C@@H]1CCN(c2n[nH]c3nccc(-c4ccc(CNC(=O)c5noc(C(C)(C)C)n5)c(F)c4)c23)C1] | 549.61 | 550.6 | 5-tert-butyl-N-[(4-{3-[(3R)-3- [(dimethylcarbamoyl) amino]pyrrolidin-1-yl]-1H- pyrazolo[3,4-b]pyridin-4-yl}- 2-fluorophenyl)methyl]-1,2,4- oxaidazole-3-carboxamide | Method E |
| 133 | 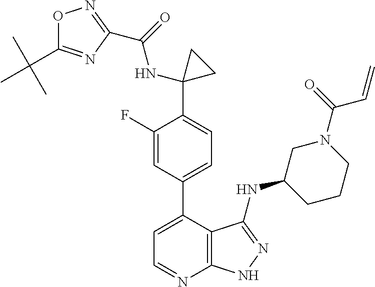 [SMILES: C=CC(=O)N1CCC[C@@H](Nc2n[nH]c3nccc(-c4ccc(C5(NC(=O)c6noc(C(C)(C)C)n6)CC5)c(F)c4)c23)C1] | 572.65 | 573.5 | 5-tert-butyl-N-{1-[2-fluoro-4-(3- {[(3R)-1-(prop-2-enoyl)piperidin-3- yl]amino}-1H- pyrazolo[3,4-b]pyridin- 4-yl)phenyl]cyclopropyl}-1,2,4- oxadiazole-3-carboxamide | Method E |
| 134 | 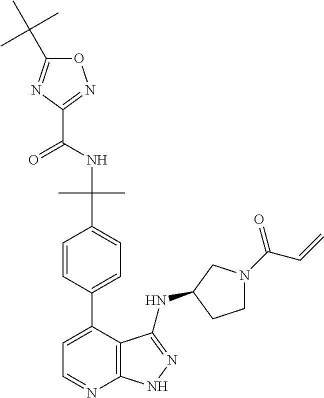 [SMILES: C=CC(=O)N1CC[C@@H](Nc2n[nH]c3nccc(-c4ccc(C(C)(C)NC(=O)c5noc(C(C)(C)C)n5)cc4)c23)C1] | 542.64 | 543.6 | 5-tert-butyl-N-{2-[4- (3-{[(3R)-1-(prop- 2-enoyl)pyrrolidin-3-yl]amino}-1H- pyrazolo[3,4-b]pyridin-4- yl)phenyl]propan-2-yl}-1,2,4- oxadiazole-3-carboxamide | Method E |
| 135 | 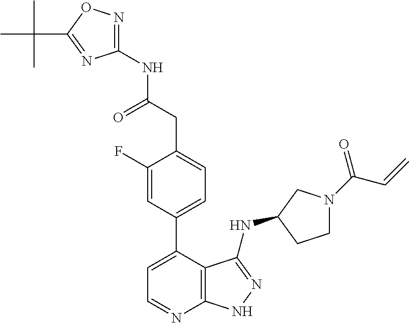 [SMILES: C=CC(=O)N1CC[C@@H](Nc2n[nH]c3nccc(-c4ccc(CC(=O)Nc5noc(C(C)(C)C)n5)c(F)c4)c23)C1] | 532.58 | 533.5 | N-(5-tert-butyl-1,2,4- oxadiazol-3-yl)-2- [2-fluoro-4-(3-{[(3R)-1-(prop-2- enoyl)pyrrolidin-3-yl]amino}-1H- pyrazolo[3,4-b]pyridin-4- yl)phenyl]acetamide | Method E |
| 136 | 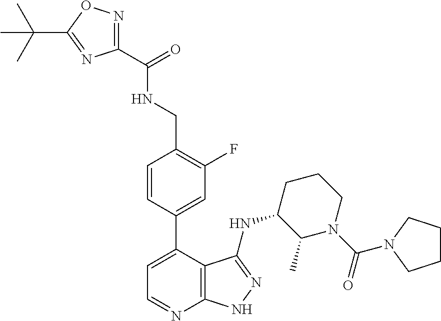 [SMILES: C[C@@H]1[C@H](Nc2n[nH]c3nccc(-c4ccc(CNC(=O)c5noc(C(C)(C)C)n5)c(F)c4)c23)CCCN1C(=O)N1CCCC1] | 603.70 | 604.3 | 5-tert-butyl-N-{[2-fluoro-4-(3- {[(2R,3R)-2-methyl-1- (pyrrolidine-1-carbonyl) piperidin-3-yl]amino}-1H- pyraozlo[3,4-b]pyridin-4- yl)phenyl]methyl}-1,2,4- oxadiazole-3-carboxamide | Method E |
| 137 | 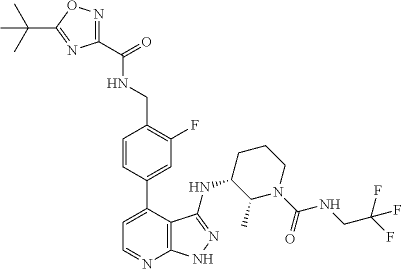 [SMILES: C[C@@H]1[C@H](Nc2n[nH]c3nccc(-c4ccc(CNC(=O)c5noc(C(C)(C)C)n5)c(F)c4)c23)CCCN1C(=O)NCC(F)(F)F] | 631.64 | 632.3 | (2R,3R)-3-{[4- (4-{[(5-tert-butyl-1,2,4- oxadiazol-3- yl)formamido]methyl}- 3-fluorophenyl)-1H- pyrazolo[3,4-b]pyridin- 3-yl]amino}-2-methyl-N- (2,2,2-trifluoroethyl)piperidine-1- carboxamide | Method E |
| 138 | 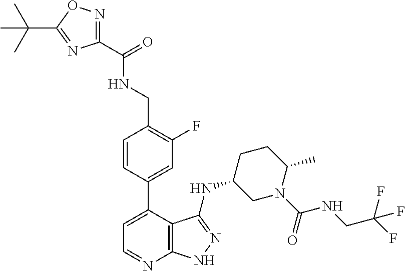 [SMILES: C[C@H]1CC[C@@H](Nc2n[nH]c3nccc(-c4ccc(CNC(=O)c5noc(C(C)(C)C)n5)c(F)c4)c23)CN1C(=O)NCC(F)(F)F] | 631.64 | 632.3 | (2S,5R)-5-{[4- (4-{[(5-tert-butyl-1,2,4- oxadiazol-3- yl)formamido]methyl}- 3-fluorophenyl)-1H-pyrazolo[3,4- b]pyridin-3-yl]amino}-2-methyl- N-(2,2,2-trifluoroethyl)piperidine- 1-carboxamide | Method E |
| 139 | 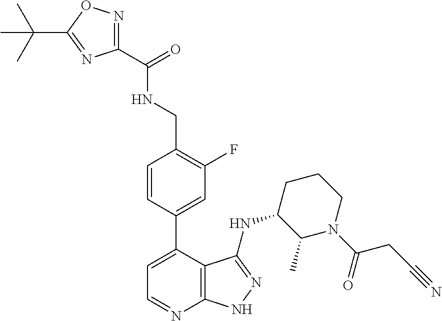 [SMILES: C[C@@H]1[C@H](Nc2n[nH]c3nccc(-c4ccc(CNC(=O)c5noc(C(C)(C)C)n5)c(F)c4)c23)CCCN1C(=O)CC#N] | 573.63 | 574.3 | 5-tert-butyl-N-{[4- (3-{[(2R,3R)-1-(2- cyanoacetyl)-2- methylpiperidin-3- yl]amino}-1H- pyrazolo[3,4-b]pyridin- 4-yl)-2-fluorophenyl]methyl}- 1,2,4-oxadiazole-3-carboxamide | Method C |
| 140 | 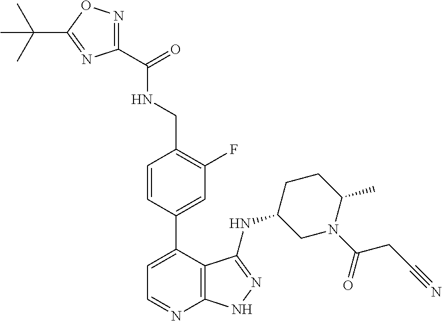 [SMILES: C[C@H]1CC[C@@H](Nc2n[nH]c3nccc(-c4ccc(CNC(=O)c5noc(C(C)(C)C)n5)c(F)c4)c23)CN1C(=O)CC#N] | 573.63 | 574.2 | 5-tert-butyl-N-{[4- (3-{[(3R,6S)-1-(2- cyanoacetyl)-6-methylpiperidin- 3-yl]amino}-1H- pyrazolo[3,4-b]pyridin- 4-yl)-2-fluorophenyl]methyl}- 1,2,4-oxadiazole-3-carboxamide | Method C |
| 141 | 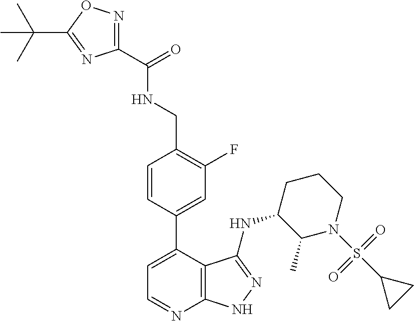 [SMILES: C[C@@H]1[C@H](Nc2n[nH]c3nccc(-c4ccc(CNC(=O)c5noc(C(C)(C)C)n5)c(F)c4)c23)CCCN1S(=O)(=O)C1CC1] | 610.71 | 611.3 | 5-tert-butyl- N-{[4-(3-{[(2R,3R)-1- (cyclopropanesulfonyl)-2- methylpiperidin-3-yl]amino}-1H- pyrazolo[3,4-b]pyridin-4-yl)-2- fluorophenyl]methyl}-1,2,4- oxadiazole-3-carboxamide | Method E |
| 142 | 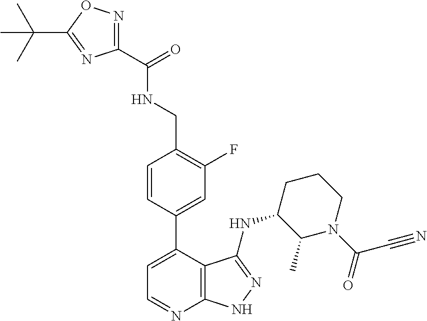 [SMILES: C[C@@H]1[C@H](Nc2n[nH]c3nccc(-c4ccc(CNC(=O)c5noc(C(C)(C)C)n5)c(F)c4)c23)CCCN1C(=O)C#N] | 545.62 | 546.3 | 5-tert-butyl-N-{[4- (3-{[(2R,3R)-1-(cyanomethyl)- 2-methylpiperidin-3- yl]amino}-1H- pyrazolo[3,4-b]pyridin- 4-yl)-2-fluorophenyl]methyl}- 1,2,4-oxadiazole-3-carboxamide | Method E |
| 143 | 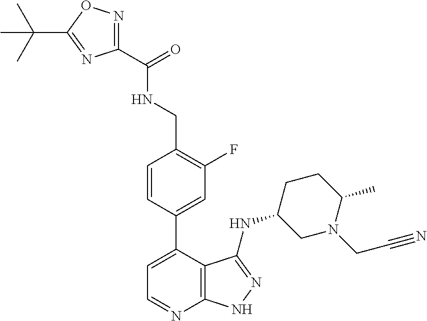 [SMILES: C[C@H]1CC[C@@H](Nc2n[nH]c3nccc(-c4ccc(CNC(=O)c5noc(C(C)(C)C)n5)c(F)c4)c23)CN1CC#N] | 545.62 | 546.3 | 5-tert-butyl-N-{[4- (3-{[(3R,6S)-1-(cyanomethyl)- 6-methylpiperidin-3- yl]amino}-1H- pyrazolo[3,4-b]pyridin- 4-yl)-2-fluorophenyl]methyl}- 1,2,4-oxadiazole-3-carboxamide | Method E |
| 144 | 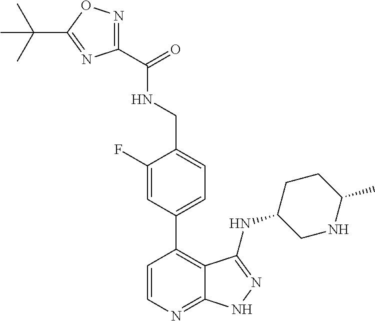 [SMILES: C[C@H]1CC[C@@H](Nc2n[nH]c3nccc(-c4ccc(CNC(=O)c5noc(C(C)(C)C)n5)c(F)c4)c23)CN1] | 505.60 | 506.5 | 5-tert-butyl-N-{[2-fluoro-4-(3- {[(3R,6S)-6-methylpiperidin-3- yl]amino}-1H- pyrazolo[3,4-b]pyridin- 4-yl)phenyl]methyl}-1,2-oxazole- 3-carboxamide | Method D |
| 145 | 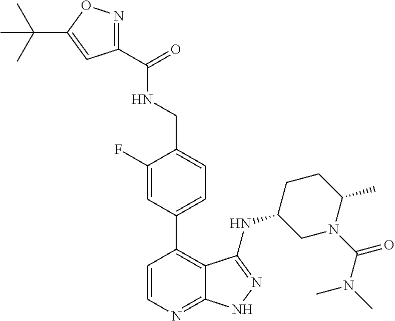 [SMILES: C[C@H]1CC[C@@H](Nc2n[nH]c3nccc(-c4ccc(CNC(=O)c5cc(C(C)(C)C)on5)c(F)c4)c23)CN1C(=O)N(C)C] | 576.68 | 577.7 | (2S,5R)-5-{[4-(4-{[(5- tert-butyl-1,2-oxazol-3- yl)formamido]methyl}-3- fluorophenyl)-1H-pyrazolo[3,4- b]pyridin-3-yl]amino}-N,N,2- trimethylpiperidine-1- carboxamide | Method E |
| 146 | 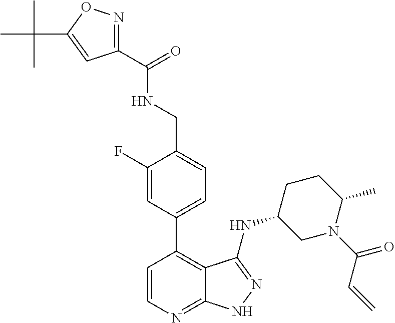 [SMILES: C=CC(=O)N1C[C@H](Nc2n[nH]c3nccc(-c4ccc(CNC(=O)c5cc(C(C)(C)C)on5)c(F)c4)c23)CC[C@@H]1C] | 559.65 | 560.6 | 5-tert-butyl-N-{[2-fluoro-4-(3- {[(3R,6S)-6-methyl-1-(prop-2- enoyl)piperidin-3-yl]amino}-1H- pyrazolo[3,4-b]pyridin-4- yl)phenyl]methyl}-1,2-oxazole-3- carboxamide | Method E |
| 147 | 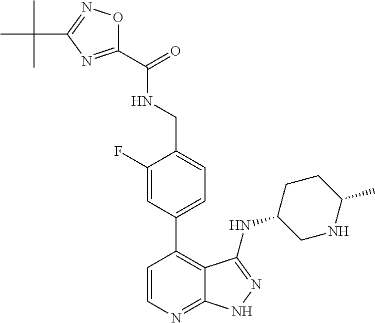 [SMILES: C[C@H]1CC[C@@H](Nc2n[nH]c3nccc(-c4ccc(CNC(=O)c5nc(C(C)(C)C)no5)c(F)c4)c23)CN1] | 506.59 | 507.5 | 3-tert-butyl-N-{[2-fluoro-4-(3- {[(3R,6S)-6-methylpiperidin-3- yl]amino}-1H- pyrazolo[3,4-b]pyridin- 4-yl)phenyl]methyl}-1,2,4- oxadiazole-5-carboxamide | Method D |
| 148 | 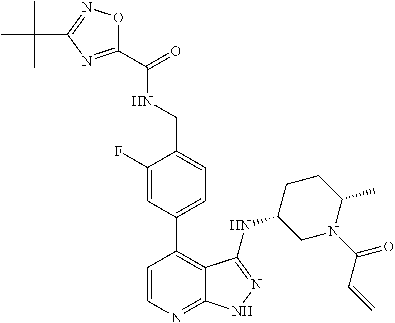 [SMILES: C=CC(=O)N1C[C@H](Nc2n[nH]c3nccc(-c4ccc(CNC(=O)c5nc(C(C)(C)C)no5)c(F)c4)c23)CC[C@@H]1C] | 560.63 | 561.5 | 3-tert-butyl-N-{[2-fluoro-4-(3- {[(3R,6S)-6-methyl-1-(prop-2- enoyl)piperidin-3-yl]amino}-1H- pyrazolo[3,4-b]pyridin-4- yl)phenyl]methyl}- 1,2,4-oxadiazole-5- carboxamide | Method E |
| 149 | 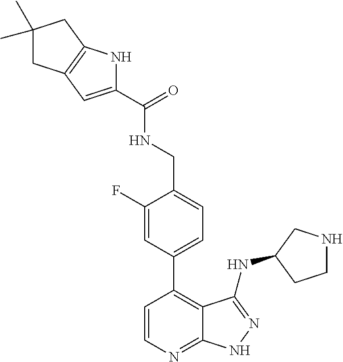 [SMILES: CC1(C)Cc2cc(C(=O)NCc3ccc(-c4ccnc5[nH]nc(N[C@@H]6CCNC6)c45)cc3F)[nH]c2C1] | 487.48 | 488.2 | N-{[2-fluoro-4- (3-{[(3R)-pyrrolidin-3-yl]amino}- 1H-pyrazolo[3,4-b]pyridin- 4-yl)phenyl]methyl}-5,5-dimethyl- 1H,4H,5H,6H- cyclopenta[b]pyrrole-2- carboxamide | Method D |
| 150 | 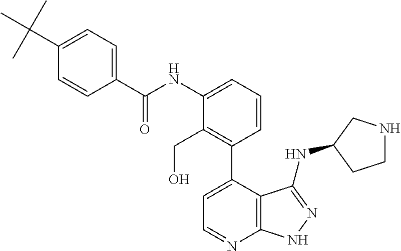 [SMILES: CC(C)(C)c1ccc(C(=O)Nc2cccc(-c3ccnc4[nH]nc(N[C@@H]5CCNC5)c34)c2CO)cc1] | 484.60 | 485.6 | 4-tert-butyl- N-[2-(hydroxymethyl)-3- (3-{[(3R)-pyrrolidin-3-yl]amino}- 1H-pyrazolo[3,4-b]pyridin-4- yl)phenyl]benzamide | Method D |
| 151 | 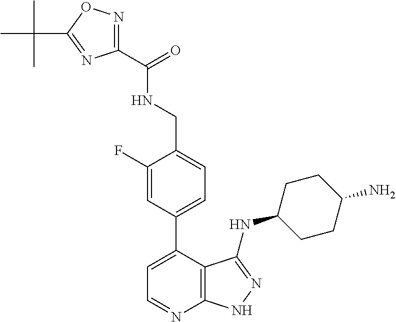 [SMILES: CC(C)(C)c1nc(C(=O)NCc2ccc(-c3ccnc4[nH]nc(N[C@H]5CC[C@H](N)CC5)c34)cc2F)no1] | 506.59 | 507.9 | 5-tert-butyl-N-{[2-fluoro-4-(3- {[(1r,4r)-4- aminocyclohexyl]amino}- 1H-pyrazolo[3,4-b]pyridin-4- yl)phenyl]methyl}-1,2,4- oxadiazole-3-carboxamide | Method D |
| 152 | 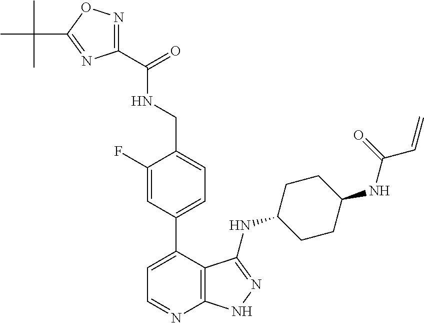 [SMILES: C=CC(=O)N[C@H]1CC[C@H](Nc2n[nH]c3nccc(-c4ccc(CNC(=O)c5noc(C(C)(C)C)n5)c(F)c4)c23)CC1] | 560.63 | 562 | 5-tert-butyl-N-{[2-fluoro-4-(3- {[(1r,4r)-4-(prop-2- enamido)cyclohexyl]amino}-1H- pyrazolo[3,4-b]pyridin-4- yl)phenyl]methyl}-1,2,4- oxadiazole-3-carboxamide | Method E |
| 153 | 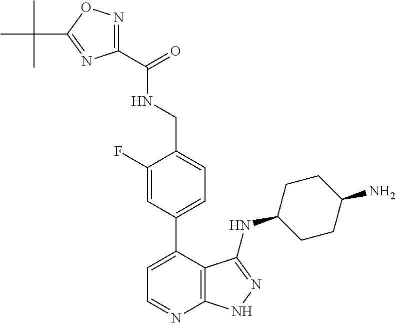 [SMILES: CC(C)(C)c1nc(C(=O)NCc2ccc(-c3ccnc4[nH]nc(N[C@H]5CC[C@@H](N)CC5)c34)cc2F)no1] | 506.59 | 507.6 | 5-tert-butyl-N-{[2-fluoro-4-(3- {[(1s,4s)-4- aminocyclohexyl]amino}- 1H-pyrazolo[3,4-b]pyridin-4- yl)phenyl]methyl}-1,2,4- oxadiazole-3-carboxamide | Method D |
| 154 | 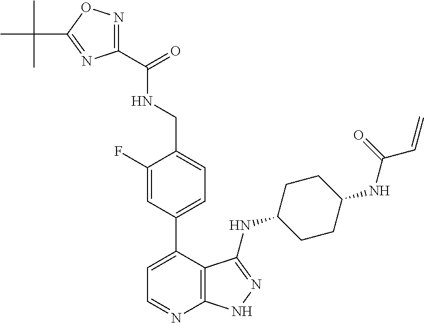 [SMILES: C=CC(=O)N[C@H]1CC[C@@H](Nc2n[nH]c3nccc(-c4ccc(CNC(=O)c5noc(C(C)(C)C)n5)c(F)c4)c23)CC1] | 560.63 | 561.9 | 5-tert-butyl-N-{[2-fluoro-4-(3- {[(1s,4s)-4-(prop-2- enamido)cyclohexyl]amino}-1H- pyrazolo[3,4-b]pyridin-4- yl)phenyl]methyl}-1,2,4- oxadiazole-3-carboxamide | Method E |
| 155 | 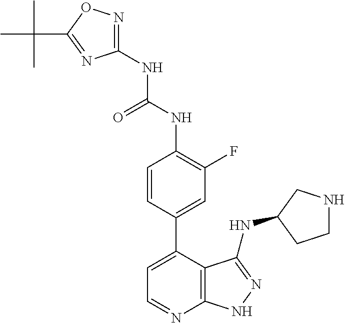 [SMILES: CC(C)(C)c1nc(NC(=O)Nc2ccc(-c3ccnc4[nH]nc(N[C@@H]5CCNC5)c34)cc2F)no1] | 479.52 | 480.9 | 3-(5-tert-butyl-1,2,4- oxadiazol-3-yl)-1- [2-fluoro-4-(3-{[(3R)-pyrrolidin- 3-yl]amino}-1H- pyrazolo[3,4-b]pyridin- 4-yl)phenyl]urea | Method D |
| 156 | 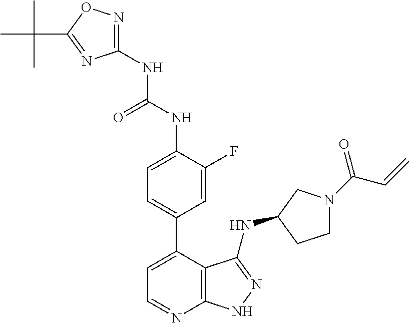 [SMILES: C=CC(=O)N1CC[C@@H](Nc2n[nH]c3nccc(-c4ccc(NC(=O)Nc5noc(C(C)(C)C)n5)c(F)c4)c23)C1] | 533.57 | 534.8 | 3-(5-tert-butyl-1,2,4- oxadiazol-3-yl)-1- [2-fluoro-4-(3-{[(3R)-1-(prop-2- enoyl)pyrrolidin-3-yl]amino}-1H- pyrazolo[3,4-b]pyridin-4- yl)phenyl]urea | Method E |
| 157 | 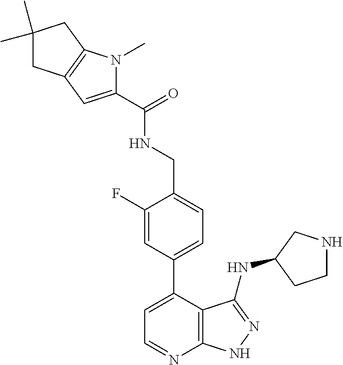 [SMILES: Cn1c(C(=O)NCc2ccc(-c3ccnc4[nH]nc(N[C@@H]5CCNC5)c34)cc2F)cc2c1CC(C)(C)C2] | 501.61 | 502.2 | N-{[2-fluoro-4- (3-{[(3R)-pyrrolidin-3- yl]amino}-1H- pyrazolo[3,4-b]pyridin- 4-yl)phenyl]methyl}-1,5,5- trimethyl-1H,4H,5H,6H- cyclopenta[b]pyrrole-2- carboxamide | Method D |
| 158 | 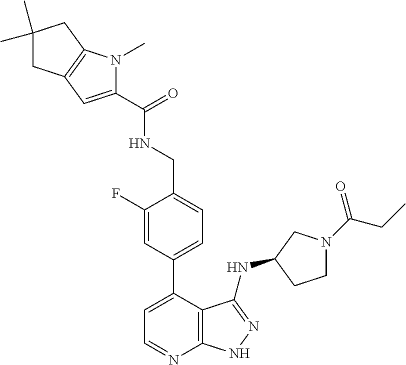 [SMILES: CCC(=O)N1CC[C@@H](Nc2n[nH]c3nccc(-c4ccc(CNC(=O)c5cc6c(n5C)CC(C)(C)C6)c(F)c4)c23)C1] | 555.66 | 556.3 | N-{[2-fluoro-4- (3-{[(3R)-1-(prop-2- enoyl)pyrrolidin-3-yl]amino}- 1H-pyrazolo[3,4-b]pyridin-4- yl)phenyl]methyl}-1,5,5- trimethyl-1H,4H,5H,6H- cyclopenta[b]pyrrole-2- carboxamide | Method E |
| 159 | 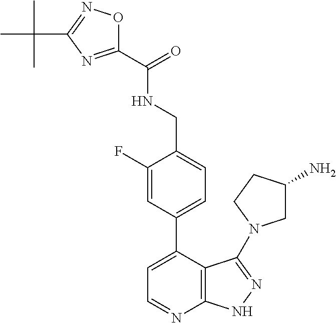 [SMILES: CC(C)(C)c1noc(C(=O)NCc2ccc(-c3ccnc4[nH]nc(N5CC[C@H](N)C5)c34)cc2F)n1] | 478.53 | 479.2 | N-[(4-{3-[(3S)-3-aminopyrrolidin- 1-yl]-1H-pyrazolo[3,4-b]pyridin- 4-yl}-2- fluorophenyl)methyl]-3-tert-butyl- 1,2,4-oxadiazole-5-carboxamide | Method D |
| 160 | 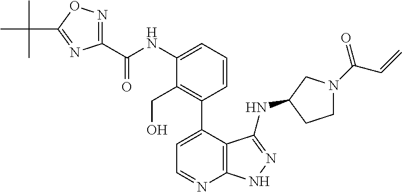 [SMILES: C=CC(=O)N1CC[C@@H](Nc2n[nH]c3nccc(-c4cccc(NC(=O)c5noc(C(C)(C)C)n5)c4CO)c23)C1] | 530.59 | 531.6 | 5-tert-butyl-N-[2- (hydroxymethyl)-3-(3-{[(3R)- 1-(prop-2-enoyl)pyrrolidin- 3-yl]amino}-1H-pyrazolo[3,4- b]pyridin-4-yl)phenyl]-1,2,4- oxadiazole-3-carboxamide | Method E |
| 161 | 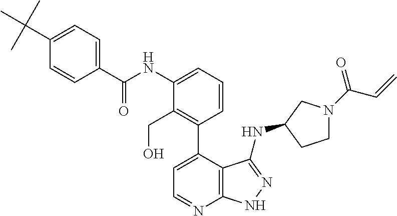 [SMILES: C=CC(=O)N1CC[C@@H](Nc2n[nH]c3nccc(-c4cccc(NC(=O)c5ccc(C(C)(C)C)cc5)c4CO)c23)C1] | 538.65 | 539.6 | 4-tert-butyl-N-[2- (hydroxymethyl)-3-(3- {[(3R)-1-(prop-2-enoyl)pyrrolidin- 3-yl]amino}-1H-pyrazolo[3,4- b]pyridin-4-yl)phenyl]benzamide | Method E |
| 162 | 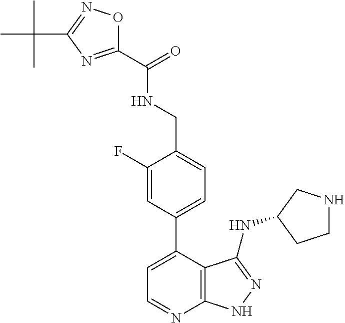 [SMILES: CC(C)(C)c1noc(C(=O)NCc2ccc(-c3ccnc4[nH]nc(N[C@H]5CCNC5)c34)cc2F)n1] | 478.53 | 479.6 | 3-tert-butyl-N-{[2- fluoro-4-(3-{[(3S)- pyrrolidin-3-yl]amino}-1H- pyraozlo[3,4-b]pyridin-4- yl)phenyl]methyl}-1,2,4- oxadiazole-5-carboxamide | Method D |
| 163 | 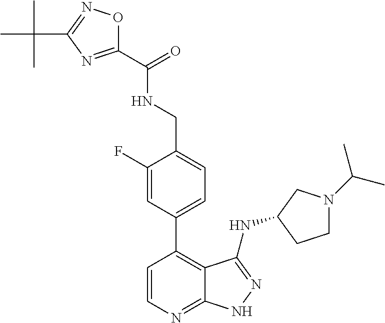 [SMILES: CC(C)N1CC[C@H](Nc2n[nH]c3nccc(-c4ccc(CNC(=O)c5nc(C(C)(C)C)no5)c(F)c4)c23)C1] | 520.61 | 521.6 | 3-tert-butyl-N-{[2- fluoro-4-(3-{[(3S)-1-(propan- 2-yl)pyrrolidin-3-yl]amino}- 1H-pyrazolo[3,4-b]pyridin-4- yl)phenyl]methyl}-1,2,4- oxadiazole-5-carboxamide | Method D |
| 164 | 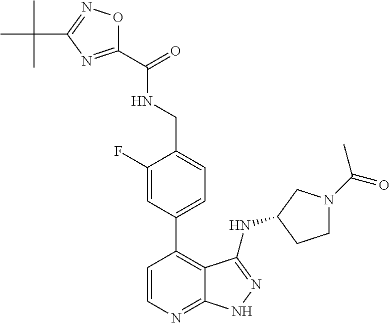 [SMILES: CC(=O)N1CC[C@H](Nc2n[nH]c3nccc(-c4ccc(CNC(=O)c5nc(C(C)(C)C)no5)c(F)c4)c23)C1] | 520.57 | 521.6 | N-{[4-(3-{[(3S)-1- acetylpyrrolidin-3-yl]amino}- 1H-pyrazolo[3,4-b]pyridin- 4-yl)-2-fluorophenyl]methyl}- 3-tert-butyl-1,2,4- oxadiazole-5-carboxamide | Method E |
| 165 | 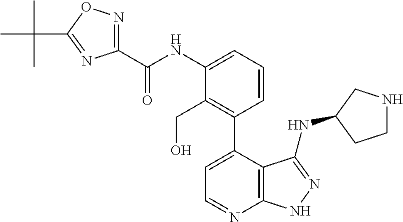 [SMILES: CC(C)(C)c1nc(C(=O)Nc2cccc(-c3ccnc4[nH]nc(N[C@@H]5CCNC5)c34)c2CO)no1] | 476.54 | 477.4 | t-tert-butyl-N-[2- (hydroxymethyl)-3-(3-{[(3R)- pyrrolidin-3-yl]amino}-1H- pyrazolo[3,4-b]pyridin- 4-yl)phenyl]- 1,2,4-oxadiazole-3-carboxamide | Method D |
| 166 | 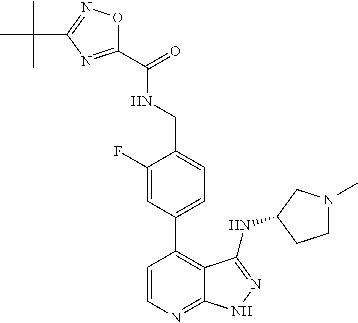 [SMILES: CN1CC[C@H](Nc2n[nH]c3nccc(-c4ccc(CNC(=O)c5nc(C(C)(C)C)no5)c(F)c4)c23)C1] | 492.56 | 493.4 | 3-tert-butyl-N-{[2- fluoro-4-(3-{[(3S)- 1-methylpyrrolidin-3-yl]amino}- 1H-pyrazolo[3,4-b]pyridin-4- yl)phenyl]methyl}-1,2,4- oxadiazole-5-carboxamide | Method D |
| 167 | 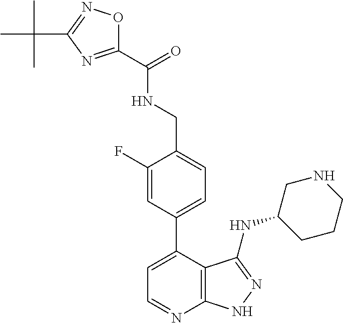 [SMILES: CC(C)(C)c1noc(C(=O)NCc2ccc(-c3ccnc4[nH]nc(N[C@H]5CCCNC5)c34)cc2F)n1] | 492.56 | 493.4 | 3-tert-butyl-N-{[2- fluoro-4-(3-{[(3S)- piperidin-3-yl]amino}-1H- pyrazolo[3,4-b]pyridin-4- yl)phenyl]methyl}-1,2,4- oxadiazole-5-carboxamide | Method D |
| 168 | 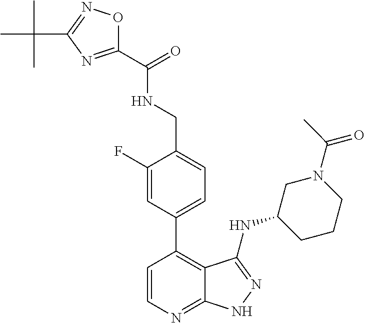 [SMILES: CC(=O)N1CCC[C@H](Nc2n[nH]c3nccc(-c4ccc(CNC(=O)c5nc(C(C)(C)C)no5)c(F)c4)c23)C1] | 534.60 | 535.7 | N-{[4-(3-{[(3S)-1- acetylpiperidin-3-yl]amino}- 1H-pyrazolo[3,4-b]pyridin- 4-yl)-2-fluorophenyl]methyl}- 3-tert-butyl-1,2,4- oxadiazole-5-carboxamide | Method E |
| 169 | 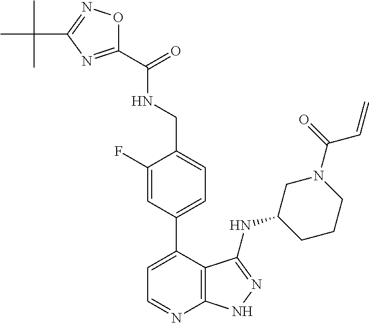 [SMILES: C=CC(=O)N1CCC[C@H](Nc2n[nH]c3nccc(-c4ccc(CNC(=O)c5nc(C(C)(C)C)no5)c(F)c4)c23)C1] | 546.61 | 547.4 | 3-tert-butyl- N-{[2-fluoro-4-(3-{[(3S)- 1-(prop-2-enoyl)piperidin- 3-yl]amino}- 1H-pyrazolo[3,4-b]pyridin-4- yl)phenyl]methyl}-1,2,4- oxadiazole-5-carboxamide | Method E |
| 170 | 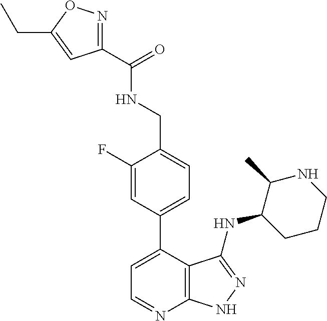 [SMILES: CCc1cc(C(=O)NCc2ccc(-c3ccnc4[nH]nc(N[C@@H]5CCCN[C@@H]5C)c34)cc2F)no1] | 477.54 | 478.3 | 5-ethyl-N-{[2-fluoro-4- (3-{[(2R,3R)-2- methylpiperidin-3-yl]amino}-1H- pyrazolo[3,4-b]pyridin-4- yl)phenyl]methyl}-1,2-oxazole-3- carboxamide | Method D |
| 171 | 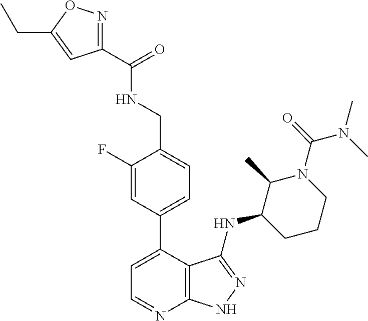 [SMILES: CCc1cc(C(=O)NCc2ccc(-c3ccnc4[nH]nc(N[C@@H]5CCCN(C(=O)N(C)C)[C@@H]5C)c34)cc2F)no1] | 548.62 | 549.4 | (2R,3R)-3-{[4-(4-{[(5-ethyl-1,2- oxazol-3-yl)formamido]methyl}- 3-fluorophenyl)-1H-pyrazolo[3,4- b]pyridin-3-yl]amino}-N,N,2- trimethylpiperidine-1-carboxamide | Method E |
| 172 | 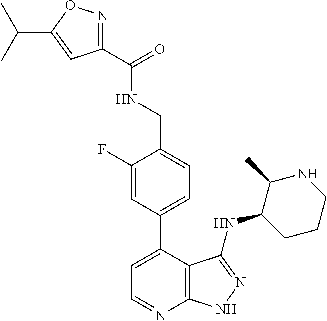 [SMILES: CC(C)c1cc(C(=O)NCc2ccc(-c3ccnc4[nH]nc(N[C@@H]5CCCN[C@@H]5C)c34)cc2F)no1] | 491.57 | 492.3 | N-{[2-fluoro-4-(3-{[(2R,3R)-2- methylpiperidin-3-yl]amino}-1H- pyrazolo[3,4-b]pyridin-4- yl)phenyl]methyl}-5- (propan-2-yl)-1,2- oxazole-3-carboxamide | Method D |
| 173 | 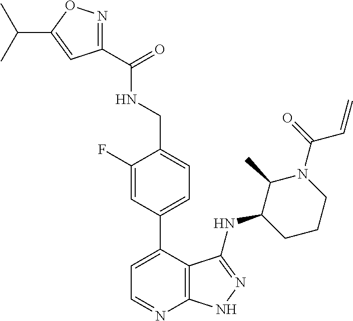 [SMILES: C=CC(=O)N1CCC[C@@H](Nc2n[nH]c3nccc(-c4ccc(CNC(=O)c5cc(C(C)C)on5)c(F)c4)c23)[C@H]1C] | 545.62 | 546.4 | N-{[2-fluoro-4-(3-{[(2R,3R)- 2-methyl-1-(prop-2-enoyl) piperidin-3-yl]amino}- 1H-pyrazolo[3,4-b]pyridin-4- yl)phenyl]methyl}-5-(propan- 2-yl)-1,2-oxazole-3-carboxamide | Method E |
| 174 | 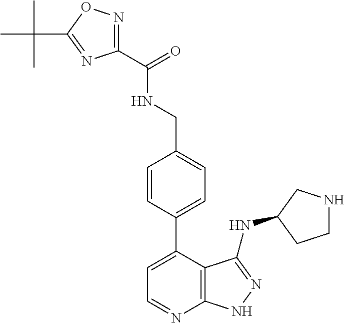 [SMILES: CC(C)(C)c1nc(C(=O)NCc2ccc(-c3ccnc4[nH]nc(N[C@@H]5CCNC5)c34)cc2)no1] | 460.54 | 461.3 | 5-tert-butyl-N-{[4-(3-{[(3R)- pyrrolidin-3-yl]amino}-1H- pyrazolo[3,4-b]pyridin-4- yl)phenyl]methyl}-1,2,4- oxadiazole-3-carboxamide | Method D |
| 175 | 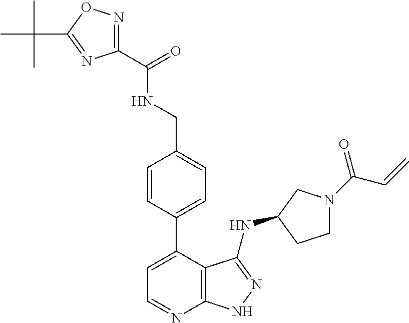 [SMILES: C=CC(=O)N1CC[C@@H](Nc2n[nH]c3nccc(-c4ccc(CNC(=O)c5noc(C(C)(C)C)n5)cc4)c23)C1] | 514.59 | 515.4 | 5-tert-butyl-N-{[4- (3-{[(3R)-1-(prop-2- enoyl)pyrrolidin-3-yl]amino}-1H- pyrazolo[3,4-b]pyridin-4- yl)phenyl]methyl}-1,2,4- oxadiazole-3-carboxamide | Method E |
| 176 | 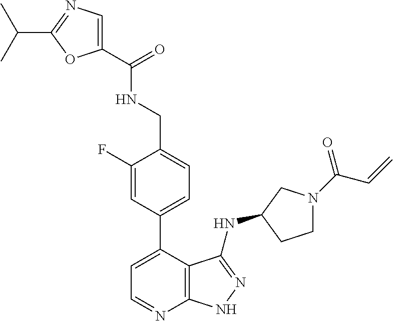 [SMILES: C=CC(=O)N1CC[C@@H](Nc2n[nH]c3nccc(-c4ccc(CNC(=O)c5cnc(C(C)C)o5)c(F)c4)c23)C1] | 517.57 | 518.6 | N-{[2-fluoro-4- (3-{[(3R)-1-(prop-2- enoyl)pyrrolidin-3-yl]amino}-1H- pyrazolo[3,4-b]pyridin-4- yl)phenyl]methyl}-2-(propan-2- yl)-1,3-oxazole-5-carboxamide | Method E |
| 177 | 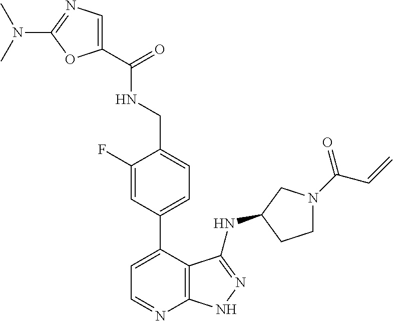 [SMILES: C=CC(=O)N1CC[C@@H](Nc2n[nH]c3nccc(-c4ccc(CNC(=O)c5cnc(N(C)C)o5)c(F)c4)c23)C1] | 518.55 | 519.4 | 2-(dimethylamino)- N-{[2-fluoro-4-(3- {[(3R)-1-(prop-2- enoyl)pyrrolidin-3- yl]amino}-1H- pyrazolo[3,4-b]pyridin- 4-yl)phenyl]methyl}- 1,3-oxazole-5-carboxamide | Method E |
| 178 | 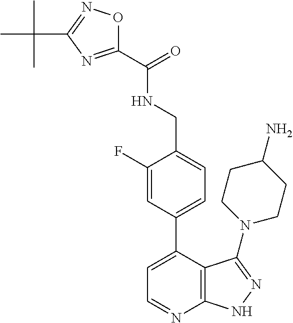 [SMILES: CC(C)(C)c1noc(C(=O)NCc2ccc(-c3ccnc4[nH]nc(N5CCC(N)CC5)c34)cc2F)n1] | 492.56 | 493.2 | N-({4-[3-(4- aminopiperidin-1-yl)-1H- pyrazolo[3,4-b]pyridin-4-yl]-2- fluorophenyl}methyl)-3-tert-butyl- 1,2,4-oxadiazole-5-carboxamide | Method D |
| 179 | 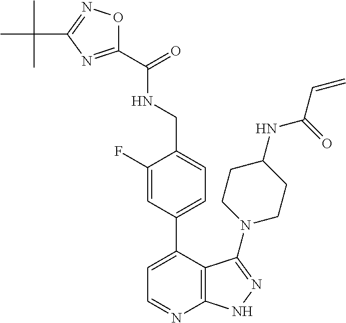 [SMILES: C=CC(=O)NC1CCN(c2n[nH]c3nccc(-c4ccc(CNC(=O)c5nc(C(C)(C)C)no5)c(F)c4)c23)CC1] | 546.61 | 547.2 | 3-tert-butyl-N-[(2-fluoro- 4-{3-[4-(prop-2- enamido)piperidin-1-yl]-1H- pyrazolo[3,4-b]pyridin-4- yl}phenyl)methyl]-1,2,4- oxadiazole-5-carboxamide | Method E |
| 180 | 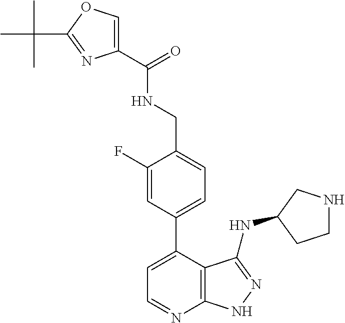 [SMILES: CC(C)(C)c1nc(C(=O)NCc2ccc(-c3ccnc4[nH]nc(N[C@@H]5CCNC5)c34)cc2F)co1] | 477.54 | 478.2 | 2-tert-butyl-N-{[2- fluoro-4-(3-{[(3R)- pyrrolidin-3-yl]amino}-1H- pyrazolo[3,4-b]pyridin-4- yl)phenyl]methyl}-1,3-oxazole-4- carboxamide | Method D |

Additional compounds that may be prepared according to the methods disclosed herein include:

| | | |
|---|---|---|
| 181 | 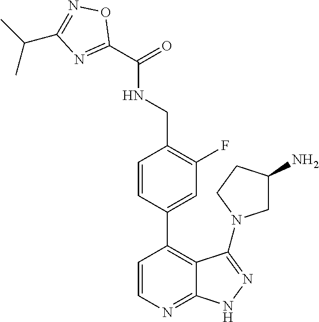 [SMILES: CC(C)c1noc(C(=O)NCc2ccc(-c3ccnc4[nH]nc(N5CC[C@@H](N)C5)c34)cc2F)n1] | N-[(4-{3-[(3R)-3- aminopyrrolidin-1-yl]-1H- pyrazolo[3,4-b]pyridin-4- yl}-2- fluorophenyl)methyl]-3- (propan-2-yl)-1,2,4- oxadiazole-5-carboxamide |
| 182 | 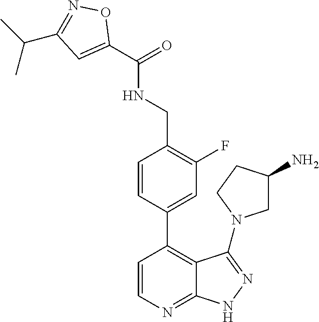 [SMILES: CC(C)c1cc(C(=O)NCc2ccc(-c3ccnc4[nH]nc(N5CC[C@@H](N)C5)c34)cc2F)on1] | N-[(4-{3-[(3R)-3- aminopyrrolidin-1-yl]-1H- pyrazolo[3,4-b]pyridin-4- yl}-2- fluorophenyl)methyl]-3- (propan-2-yl)-1,2-oxazole- 5-carboxamide |
| 184 | 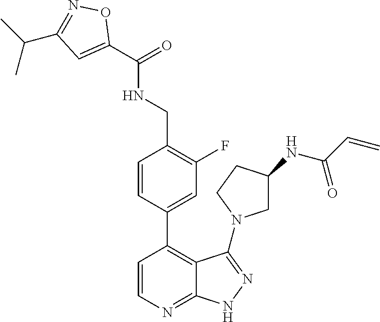 [SMILES: C=CC(=O)N[C@@H]1CCN(c2n[nH]c3nccc(-c4ccc(CNC(=O)c5cc(C(C)C)no5)c(F)c4)c23)C1] | N-{[2-fluoro-4-(3-{(3R)-3- [(prop-2- enoyl)amino]pyrrolidin-1- yl}-1H-pyrazolo[3,4- b]pyridin-4- yl)phenyl]methyl}-3- (propan-2-yl)-1,2-oxazole- 5-carboxamide |
| 185 | 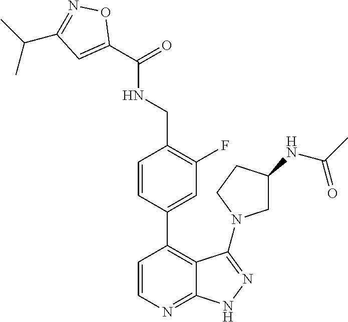 [SMILES: CC(=O)N[C@@H]1CCN(c2n[nH]c3nccc(-c4ccc(CNC(=O)c5cc(C(C)C)no5)c(F)c4)c23)C1] | N-[(4-{3-[(3R)-3- acetamidopyrrolidin-1-yl]- 1H-pyrazolo[3,4-b]pyridin- 4-yl}-2- fluorophenyl)methyl]-3- (propan-2-yl)-1,2-oxazole- 5-carboxamide |
| 188 | 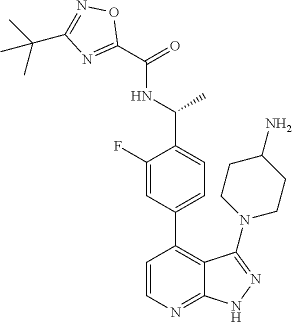 [SMILES: C[C@@H](NC(=O)c1nc(C(C)(C)C)no1)c1ccc(-c2ccnc3[nH]nc(N4CCC(N)CC4)c23)cc1F] | N-[(1R)-1-{4-[3-(4- aminopiperidin-1-yl)-1H- pyrazolo[3,4-b]pyridin-4- yl]-2-fluorophenyl}ethyl]- 3-tert-butyl-1,2,4- oxadiazole-5-carboxamide |
| 191 | 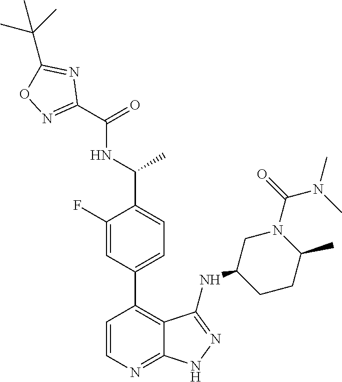 [SMILES: C[C@@H](NC(=O)c1noc(C(C)(C)C)n1)c1ccc(-c2ccnc3[nH]nc(N[C@@H]4CC[C@H](C)N(C(=O)N(C)C)C4)c23)cc1F] | (2S,5R)-5-{[4-(4-{(1R)-1- [(5-tert-butyl-1,2,4- oxadiazole-3- carbonyl)amino]ethyl}-3- fluorophenyl)-1H- pyrazolo[3,4-b]pyridin-3- yl]amino}-N,N,2- trimethylpiperidine-1- carboxamide |
| 192 | 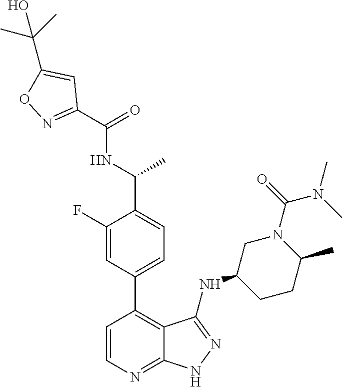 [SMILES: C[C@@H](NC(=O)c1cc(C(C)(C)O)on1)c1ccc(-c2ccnc3[nH]nc(N[C@@H]4CC[C@H](C)N(C(=O)N(C)C)C4)c23)cc1F] | (2S,5R)-5-[(4-{3-fluoro-4- [(1R)-1-{[5-(2- hydroxypropan-2-yl)-1,2- oxazole-3- carbonyl]amino}ethyl] phenyl}-1H-pyrazolo[3,4- b]pyridin-3-yl)amino]- N,N,2-trimethylpiperidine- 1-carboxamide |
| 193 | 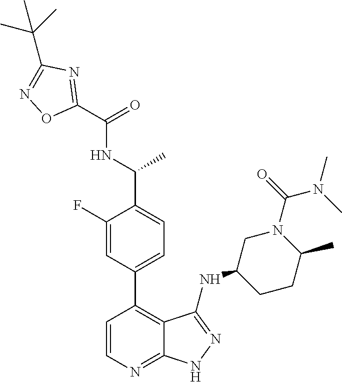 [SMILES: C[C@@H](NC(=O)c1nc(C(C)(C)C)no1)c1ccc(-c2ccnc3[nH]nc(N[C@@H]4CC[C@H](C)N(C(=O)N(C)C)C4)c23)cc1F] | (2S,5R)-5-{[4-(4-{(1R)-1- [(3-tert-butyl-1,2,4- oxadiazole-5- carbonyl)amino]ethyl}-3- fluorophenyl)-1H- pyrazolo[3,4-b]pyridin-3- yl]amino}-N,N,2- trimethylpiperidine-1- carboxamide |
| 194 | 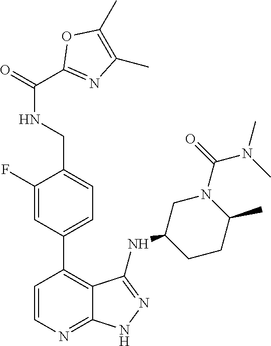 [SMILES: Cc1nc(C(=O)NCc2ccc(-c3ccnc4[nH]nc(N[C@@H]5CC[C@H](C)N(C(=O)N(C)C)C5)c34)cc2F)oc1C] | (2S,5R)-5-{[4-(4-{[(4,5- dimethyl-1,3-oxazole-2- carbonyl)amino]methyl}-3- fluorophenyl)-1H- pyrazolo[3,4-b]pyridin-3- yl]amino}-N,N,2- trimethylpiperidine-1- carboxamide |
| 196 | 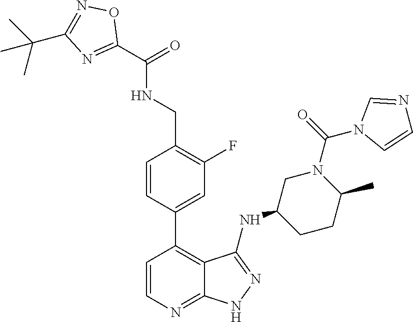 [SMILES: C[C@H]1CC[C@@H](Nc2n[nH]c3nccc(-c4ccc(CNC(=O)c5nc(C(C)(C)C)no5)c(F)c4)c23)CN1C(=O)n1ccnc1] | 3-tert-butyl-N-{[2-fluoro-4- (3-{[(3R,6S)-1-(1H- imidazole-1-carbonyl)-6- methylpiperidin-3- yl]amino}-1H- pyrazolo[3,4-b]pyridin-4- yl)phenyl]methyl}-1,2,4- oxadiazole-5-carboxamide |
| 198 | 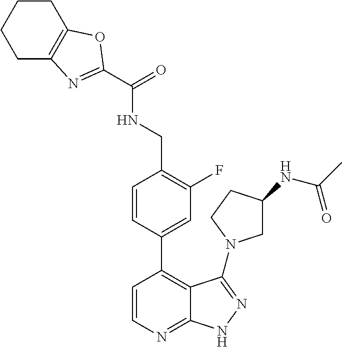 [SMILES: CC(=O)N[C@@H]1CCN(c2n[nH]c3nccc(-c4ccc(CNC(=O)c5nc6c(o5)CCCC6)c(F)c4)c23)C1] | N-[(4-{3-[(3R)-3- acetamidopyrrolidin-1-yl]- 1H-pyrazolo[3,4-b]pyridin- 4-yl}-2- fluorophenyl)methyl]- 4,5,6,7-tetrahydro-1,3- benzoxazole-2- carboxamide |
| 200 | 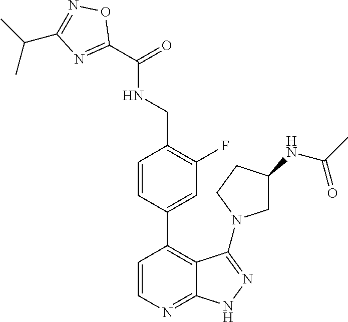 [SMILES: CC(=O)N[C@@H]1CCN(c2n[nH]c3nccc(-c4ccc(CNC(=O)c5nc(C(C)C)no5)c(F)c4)c23)C1] | N-[(4-{3-[(3R)-3- acetamidopyrrolidin-1-yl]- 1H-pyrazolo[3,4-b]pyridin- 4-yl}-2- fluorophenyl)methyl]-3- (propan-2-yl)-1,2,4- oxadiazole-5-carboxamide |
| 201 | 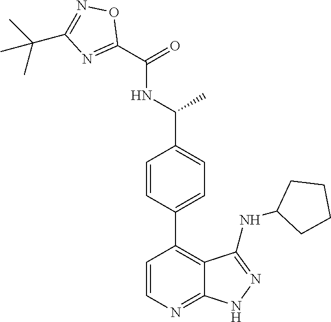 [SMILES: C[C@@H](NC(=O)c1nc(C(C)(C)C)no1)c1ccc(-c2ccnc3[nH]nc(NC4CCCC4)c23)cc1] | 3-tert-butyl-N-[(1R)-1-{4- [3-(cyclopentylamino)-1H- pyrazolo[3,4-b]pyridin-4- yl]phenyl}ethyl]-1,2,4- oxadiazole-5-carboxamide |
| 202 | 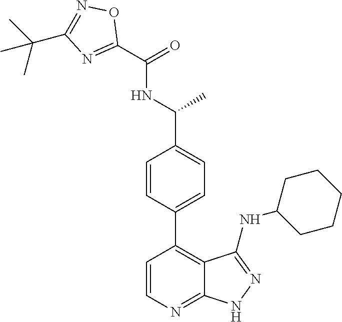 [SMILES: C[C@@H](NC(=O)c1nc(C(C)(C)C)no1)c1ccc(-c2ccnc3[nH]nc(NC4CCCCC4)c23)cc1] | 3-tert-butyl-N-[(1R)-1-{4- [3-(cyclohexylamino)-1H- pyrazolo[3,4-b]pyridin-4- yl]phenyl}ethyl]-1,2,4- oxadiazole-5-carboxamide |
| 203 | 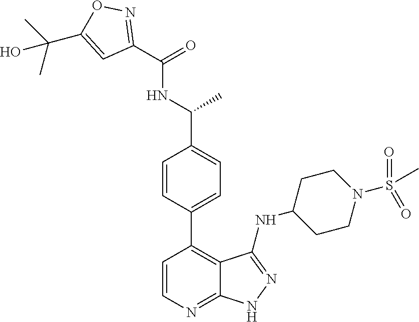 [SMILES: C[C@@H](NC(=O)c1cc(C(C)(C)O)on1)c1ccc(-c2ccnc3[nH]nc(NC4CCN(S(C)(=O)=O)CC4)c23)cc1] | 5-(2-hydroxypropan-2-yl)- N-{(1R)-1-[4-(3-{[1- (methanesulfonyl)piperidin- 4-yl]amino}-1H- pyrazolo[3,4-b]pyridin-4- yl)phenyl]ethyl}-1,2- oxazole-3-carboxamide |
| 204 | 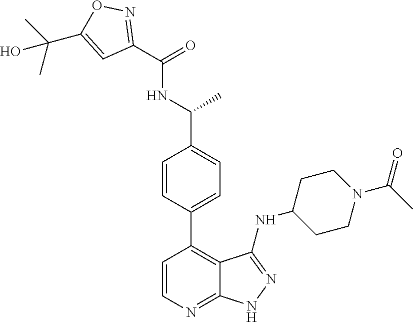 [SMILES: CC(=O)N1CCC(Nc2n[nH]c3nccc(-c4ccc([C@@H](C)NC(=O)c5cc(C(C)(C)O)on5)cc4)c23)CC1] | N-[(1R)-1-(4-{3-[(1- acetylpiperidin-4- yl)amino]-1H- pyrazolo[3,4-b]pyridin-4- yl}phenyl)ethyl]-5-(2- hydroxypropan-2-yl)-1,2- oxazole-3-carboxamide |
| 207 | 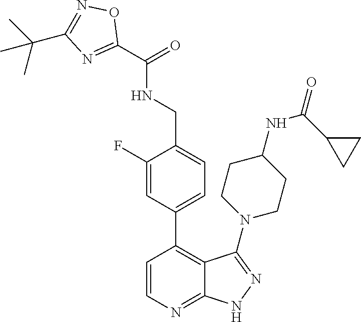 [SMILES: CC(C)(C)c1noc(C(=O)NCc2ccc(-c3ccnc4[nH]nc(N5CCC(NC(=O)C6CC6)CC5)c34)cc2F)n1] | 3-tert-butyl-N-{[4-(3-{4- [(cyclopropanecarbonyl) amino]piperidin-1-yl}-1H- pyrazolo[3,4-b]pyridin-4- yl)-2- fluorophenyl]methyl}- 1,2,4-oxadiazole-5- carboxamide |
| 208 | 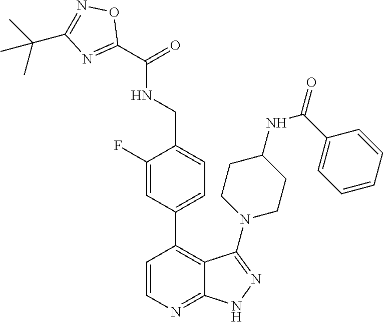 [SMILES: CC(C)(C)c1noc(C(=O)NCc2ccc(-c3ccnc4[nH]nc(N5CCC(NC(=O)c6ccccc6)CC5)c34)cc2F)n1] | N-({4-[3-(4- benzamidopiperidin-1-yl)- 1H-pyrazolo[3,4-b]pyridin- 4-yl]-2- fluorophenyl}methyl)-3- tert-butyl-1,2,4-oxadiazole- 5-carboxamide |
| 209 | 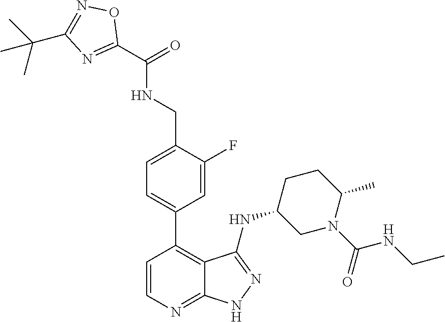 [SMILES: CCNC(=O)N1C[C@H](Nc2n[nH]c3nccc(-c4ccc(CNC(=O)c5nc(C(C)(C)C)no5)c(F)c4)c23)CC[C@@H]1C] | (2S,5R)-5-{[4-(4-{[(3-tert- butyl-1,2,4-oxadiazole-5- carbonyl)amino]methyl}-3- fluorophenyl)-1H- pyrazolo[3,4-b]pyridin-3- yl]amino}-N-ethyl-2- methylpiperidine-1- carboxamide |
| 217 | 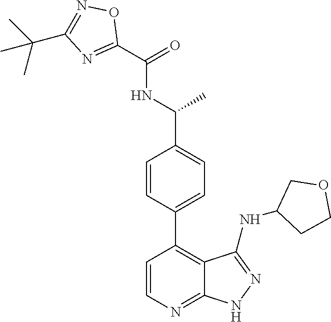 [SMILES: C[C@@H](NC(=O)c1nc(C(C)(C)C)no1)c1ccc(-c2ccnc3[nH]nc(NC4CCOC4)c23)cc1] | 3-tert-butyl-N-[(1R)-1-(4- {3-[(oxolan-3-yl)amino]- 1H-pyrazolo[3,4-b]pyridin- 4-yl}phenyl)ethyl]-1,2,4- oxadiazole-5-carboxamide |
| 218 | 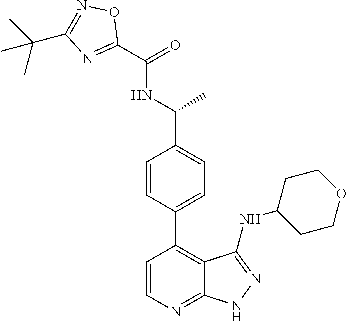 [SMILES: C[C@@H](NC(=O)c1nc(C(C)(C)C)no1)c1ccc(-c2ccnc3[nH]nc(NC4CCOCC4)c23)cc1] | 3-tert-butyl-N-[(1R)-1-(4- {3-[(oxan-4-yl)amino]-1H- pyrazolo[3,4-b]pyridin-4- yl}phenyl)ethyl]-1,2,4- oxadiazole-5-carboxamide |
| 219 | 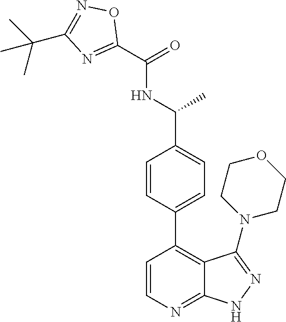 [SMILES: C[C@@H](NC(=O)c1nc(C(C)(C)C)no1)c1ccc(-c2ccnc3[nH]nc(N4CCOCC4)c23)cc1] | 3-tert-butyl-N-[(1R)-1-{4- [3-(morpholin-4-yl)-1H- pyrazolo[3,4-b]pyridin-4- yl]phenyl}ethyl]-1,2,4- oxadiazole-5-carboxamide |
| 220 | 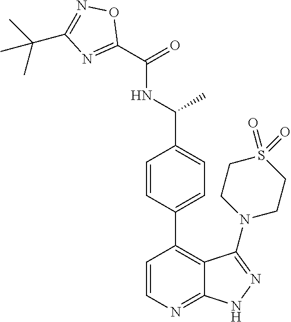 [SMILES: C[C@@H](NC(=O)c1nc(C(C)(C)C)no1)c1ccc(-c2ccnc3[nH]nc(N4CCS(=O)(=O)CC4)c23)cc1] | 3-tert-butyl-N-[(1R)-1-{4- [3-(1,1-dioxo-1λ⁶- thiomorpholin-4-yl)-1H- pyrazolo[3,4-b]pyridin-4- yl]phenyl}ethyl]-1,2,4- oxadiazole-5-carboxamide |
| 224 | 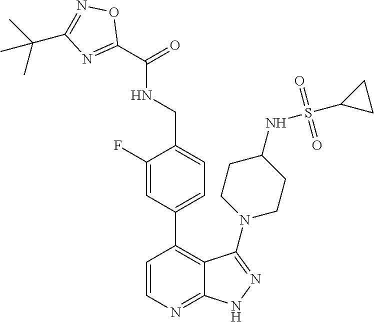 [SMILES: CC(C)(C)c1noc(C(=O)NCc2ccc(-c3ccnc4[nH]nc(N5CCC(NS(=O)(=O)C6CC6)CC5)c34)cc2F)n1] | 3-tert-butyl-N-{[4-(3-{4- [(cyclopropanesulfonyl) amino]piperidin-1-yl}-1H- pyrazolo[3,4-b]pyridin-4- yl)-2- fluorophenyl]methyl}- 1,2,4-oxadiazole-5- carboxamide |
| 226 | 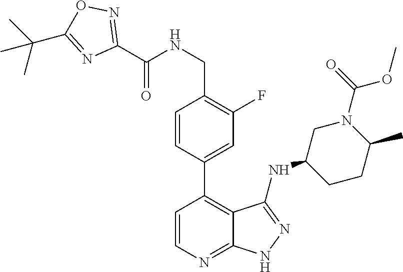 [SMILES: COC(=O)N1C[C@H](Nc2n[nH]c3nccc(-c4ccc(CNC(=O)c5noc(C(C)(C)C)n5)c(F)c4)c23)CC[C@@H]1C] | methyl (2S,5R)-5-{[4-(4- {[(5-tert-butyl-1,2,4- oxadiazole-3- carbonyl)amino]methyl}-3- fluorophenyl)-1H- pyrazolo[3,4-b]pyridin-3- yl]amino}-2- methylpiperidine-1- carboxylate |
| 227 | 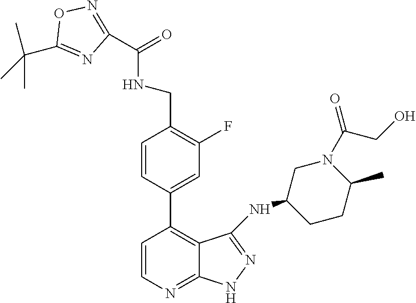 [SMILES: C[C@H]1CC[C@@H](Nc2n[nH]c3nccc(-c4ccc(CNC(=O)c5noc(C(C)(C)C)n5)c(F)c4)c23)CN1C(=O)CO] | 5-tert-butyl-N-{[2-fluoro-4- (3-{[(3R,6S)-1- (hydroxyacetyl)-6- methylpiperidin-3- yl]amino}-1H- pyrazolo[3,4-b]pyridin-4- yl)phenyl]methyl}-1,2,4- oxadiazole-3-carboxamide |
| 229 | 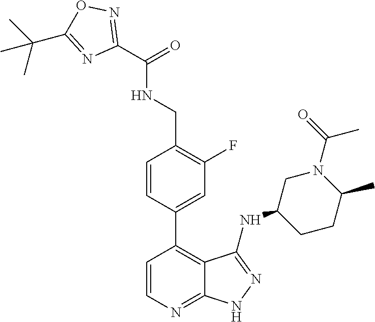 [SMILES: CC(=O)N1C[C@H](Nc2n[nH]c3nccc(-c4ccc(CNC(=O)c5noc(C(C)(C)C)n5)c(F)c4)c23)CC[C@@H]1C] | N-{[4-(3-{[(3R,6S)-1- acetyl-6-methylpiperidin-3- yl]amino}-1H- pyrazolo[3,4-b]pyridin-4- yl)-2- fluorophenyl]methyl}-5- tert-butyl-1,2,4-oxadiazole- 3-carboxamide |
| 230 | 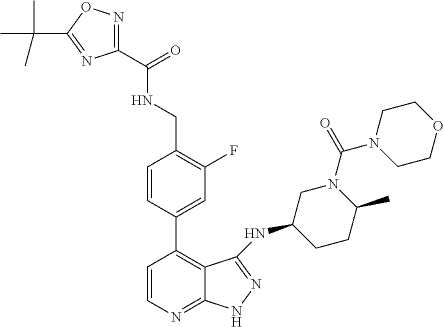 [SMILES: C[C@H]1CC[C@@H](Nc2n[nH]c3nccc(-c4ccc(CNC(=O)c5noc(C(C)(C)C)n5)c(F)c4)c23)CN1C(=O)N1CCOCC1] | 5-tert-butyl-N-{[2-fluoro-4- (3-{[(3R,6S)-6-methyl-1- (morpholine-4- carbonyl)piperidin-3- yl]amino}-1H- pyrazolo[3,4-b]pyridin-4- yl)phenyl]methyl}-1,2,4- oxadiazole-3-carboxamide |
| 238 | 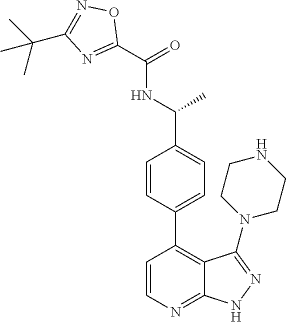 [SMILES: C[C@@H](NC(=O)c1nc(C(C)(C)C)no1)c1ccc(-c2ccnc3[nH]nc(N4CCNCC4)c23)cc1] | 3-tert-butyl-N-[(1R)-1-{4- [3-(piperazin-1-yl)-1H- pyrazolo[3,4-b]pyridin-4- yl]phenyl}ethyl]-1,2,4- oxadiazole-5-carboxamide |
| 239 | 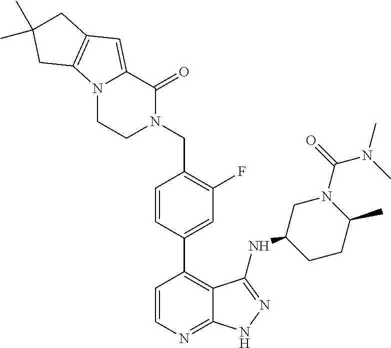 | (2S,5R)-5-[(4-{4-[(7,7- dimethyl-1-oxo-1,3,4,6,7,8- hexahydro-2H- cyclopenta[4,5]pyrrolo[1,2- a]pyrazin-2-yl)methyl]-3- fluorophenyl}-1H- pyrazolo[3,4-b]pyridin-3- yl)amino]-N,N,2- trimethylpiperidine-1- carboxamide |
| 240 | 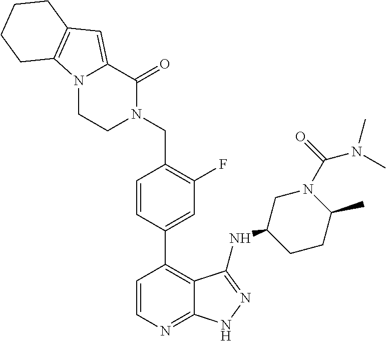 [SMILES: C[C@H]1CC[C@@H](Nc2n[nH]c3nccc(-c4ccc(CN5CCn6c(cc7c6CCCC7)C5=O)c(F)c4)c23)CN1C(=O)N(C)C] | (2S,5R)-5-[(4-{3-fluoro-4- [(1-oxo-3,4,6,7,8,9- hexahydropyrazino[1,2- a]indol-2(1H)- yl)methyl]phenyl}-1H- pyrazolo[3,4-b]pyridin-3- yl)amino]-N,N,2- trimethylpiperidine-1- carboxamide |
| 241 | 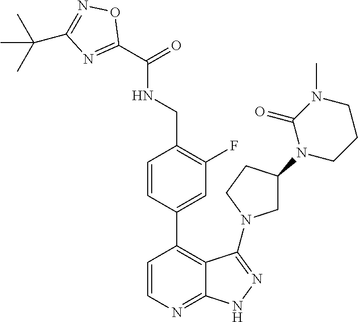 [SMILES: CN1CCCN([C@@H]2CCN(c3n[nH]c4nccc(-c5ccc(CNC(=O)c6nc(C(C)(C)C)no6)c(F)c5)c34)C2)C1=O] | 3-tert-butyl-N-[(2-fluoro-4- {3-[(3R)-3-(3-methyl-2- oxo-1,3-diazinan-1- yl)pyrrolidin-1-yl]-1H- pyrazolo[3,4-b]pyridin-4- yl}phenyl)methyl]-1,2,4- oxadiazole-5-carboxamide |
| 243 | 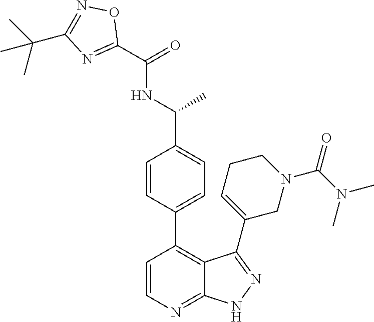 [SMILES: C[C@@H](NC(=O)c1nc(C(C)(C)C)no1)c1ccc(-c2ccnc3[nH]nc(C4=CCCN(C(=O)N(C)C)C4)c23)cc1] | 5-[4-(4-{(1R)-1-[(3-tert- butyl-1,2,4-oxadiazole-5- carbonyl)amino]ethyl} phenyl)-1H-pyrazolo[3,4- b]pyridin-3-yl]-N,N- dimethyl-3,6- dihydropyridine-1(2H)- carboxamide |
| 244 | 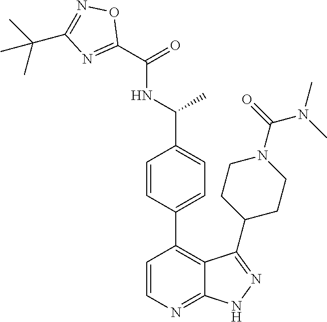 [SMILES: C[C@@H](NC(=O)c1nc(C(C)(C)C)no1)c1ccc(-c2ccnc3[nH]nc(C4CCN(C(=O)N(C)C)CC4)c23)cc1] | 4-[4-(4-{(1R)-1-[(3-tert- butyl-1,2,4-oxadiazole-5- carbonyl)amino]ethyl} phenyl)-1H-pyrazolo[3,4- b]pyridin-3-yl]-N,N- dimethylpiperidine-1- carboxamide |
| 245 | 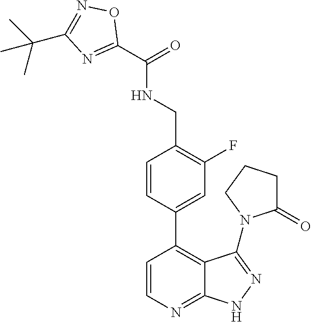 [SMILES: CC(C)(C)c1noc(C(=O)NCc2ccc(-c3ccnc4[nH]nc(N5CCCC5=O)c34)cc2F)n1] | 3-tert-butyl-N-({2-fluoro-4- [3-(2-oxopyrrolidin-1-yl)- 1H-pyrazolo[3,4-b]pyridin- 4-yl]phenyl}methyl)-1,2,4- oxadiazole-5-carboxamide |
| 250 | 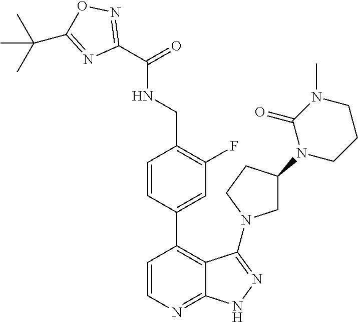 [SMILES: CN1CCCN([C@@H]2CCN(c3n[nH]c4nccc(-c5ccc(CNC(=O)c6noc(C(C)(C)C)n6)c(F)c5)c34)C2)C1=O] | 5-tert-butyl-N-[(2-fluoro-4- {3-[(3R)-3-(3-methyl-2- oxo-1,3-diazinan-1- yl)pyrrolidin-1-yl]-1H- pyrazolo[3,4-b]pyridin-4- yl}phenyl)methyl]-1,2,4- oxadiazole-3-carboxamide |
| 251 | 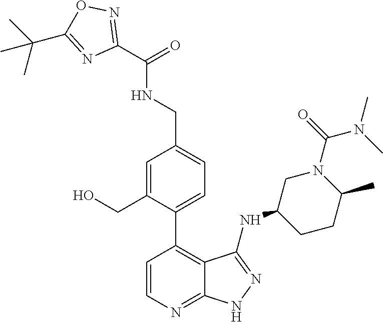 [SMILES: C[C@H]1CC[C@@H](Nc2n[nH]c3nccc(-c4ccc(CNC(=O)c5noc(C(C)(C)C)n5)cc4CO)c23)CN1C(=O)N(C)C] | (2S,5R)-5-({4-[4-{[(5-tert- butyl-1,2,4-oxadiazole-3- carbonyl)amino]methyl}-2- (hydroxymethyl)phenyl]- 1H-pyrazolo[3,4-b]pyridin- 3-yl}amino)-N,N,2- trimethylpiperidine-1- carboxamide |
| 252 | 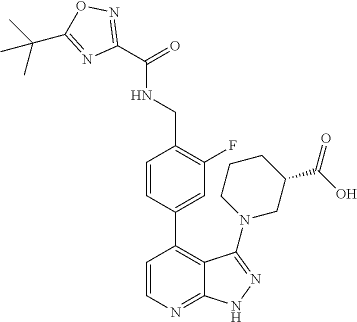 [SMILES: CC(C)(C)c1nc(C(=O)NCc2ccc(-c3ccnc4[nH]nc(N5CCC[C@H](C(=O)O)C5)c34)cc2F)no1] | (3S)-1-[4-(4-{[(5-tert- butyl-1,2,4-oxadiazole-3- carbonyl)amino]methyl}-3- fluorophenyl)-1H- pyrazolo[3,4-b]pyridin-3- yl]piperidine-3-carboxylic acid |
| 254 | 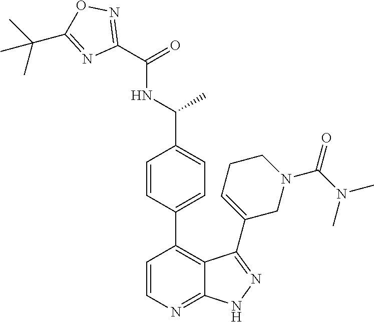 [SMILES: C[C@@H](NC(=O)c1noc(C(C)(C)C)n1)c1ccc(-c2ccnc3[nH]nc(C4=CCCN(C(=O)N(C)C)C4)c23)cc1] | 5-[4-(4-{(1R)-1-[(5-tert- butyl-1,2,4-oxadiazole-3- carbonyl)amino]ethyl} phenyl)-1H-pyrazolo[3,4- b]pyridin-3-yl]-N,N- dimethyl-3,6- dihydropyridine-1(2H)- carboxamide |
| 255 | 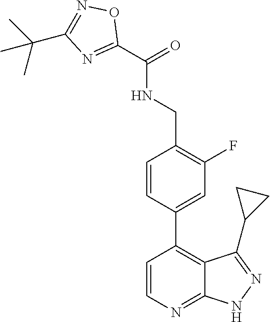 [SMILES: CC(C)(C)c1noc(C(=O)NCc2ccc(-c3ccnc4[nH]nc(C5CC5)c34)cc2F)n1] | 3-tert-butyl-N-{[4-(3- cyclopropyl-1H- pyrazolo[3,4-b]pyridin-4- yl)-2- fluorophenyl]methyl}- 1,2,4-oxadiazole-5- carboxamide |
| 256 | 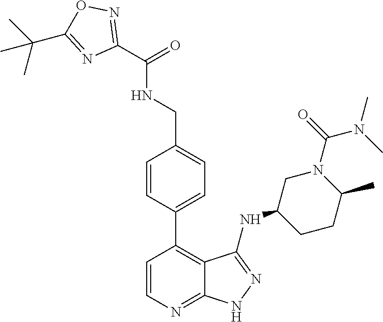 [SMILES: C[C@H]1CC[C@@H](Nc2n[nH]c3nccc(-c4ccc(CNC(=O)c5noc(C(C)(C)C)n5)cc4)c23)CN1C(=O)N(C)C] | (2S,5R)-5-{[4-(4-{[(5-tert- butyl-1,2,4-oxadiazole-3- carbonyl)amino]methyl} phenyl)-1H-pyrazolo[3,4- b]pyridin-3-yl]amino}- N,N,2-trimethylpiperidine- 1-carboxamide |
| 257 | 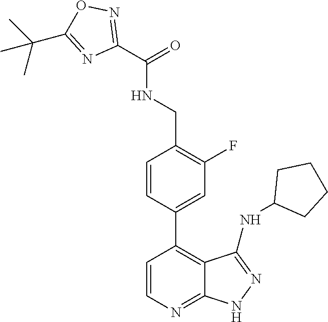 [SMILES: CC(C)(C)c1nc(C(=O)NCc2ccc(-c3ccnc4[nH]nc(NC5CCCC5)c34)cc2F)no1] | 5-tert-butyl-N-({4-[3- (cyclopentylamino)-1H- pyrazolo[3,4-b]pyridin-4- yl]-2- fluorophenyl}methyl)- 1,2,4-oxadiazole-3- carboxamide |
| 262 | 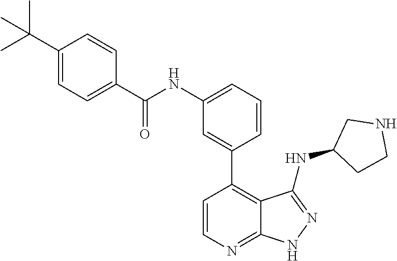 [SMILES: CC(C)(C)c1ccc(C(=O)Nc2cccc(-c3ccnc4[nH]nc(N[C@@H]5CCNC5)c34)c2)cc1] | (R)-4-tert-butyl-N-(3-{3- [(pyrrolidin-3-yl)amino]- 1H-pyrazolo[3,4-b]pyridin- 4-yl}phenyl)benzamide |
| 263 | 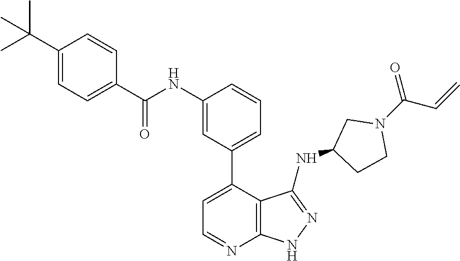 [SMILES: C=CC(=O)N1CC[C@@H](Nc2n[nH]c3nccc(-c4cccc(NC(=O)c5ccc(C(C)(C)C)cc5)c4)c23)C1] | 4-tert-butyl-N-[3-(3-{[(3R)- 1-(prop-2-enoyl)pyrrolidin- 3-yl]amino}-1H- pyrazolo[3,4-b]pyridin-4- yl)phenyl]benzamide |
| 264 | 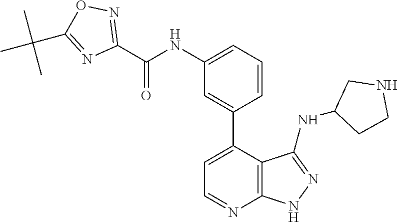 [SMILES: CC(C)(C)c1nc(C(=O)Nc2cccc(-c3ccnc4[nH]nc(NC5CCNC5)c34)c2)no1] | 5-tert-butyl-N-(3-{3- [(pyrrolidin-3-yl)amino]- 1H-pyrazolo[3,4-b]pyridin- 4-yl}phenyl)-1,2,4- oxadiazole-3-carboxamide |
| 265 | 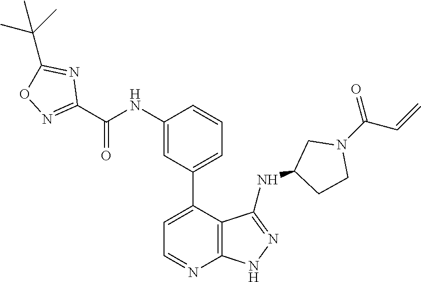 [SMILES: C=CC(=O)N1CC[C@@H](Nc2n[nH]c3nccc(-c4cccc(NC(=O)c5noc(C(C)(C)C)n5)c4)c23)C1] | 5-tert-butyl-N-[3-(3-{[(3R)- 1-(prop-2-enoyl)pyrrolidin- 3-yl]amino}-1H- pyrazolo[3,4-b]pyridin-4- yl)phenyl]-1,2,4- oxadiazole-3-carboxamide |
| 266 | 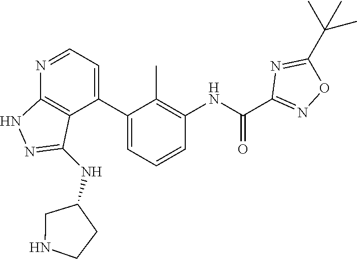 [SMILES: Cc1c(NC(=O)c2noc(C(C)(C)C)n2)cccc1-c1ccnc2[nH]nc(N[C@@H]3CCNC3)c12] | 5-tert-butyl-N-[2-methyl-3- (3-{[(3R)-pyrrolidin-3- yl]amino}-1H- pyrazolo[3,4-b]pyridin-4- yl)phenyl]-1,2,4- oxadiazole-3-carboxamide |
| 267 | 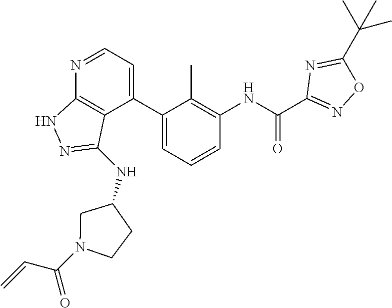 [SMILES: C=CC(=O)N1CC[C@@H](Nc2n[nH]c3nccc(-c4cccc(NC(=O)c5noc(C(C)(C)C)n5)c4C)c23)C1] | 5-tert-butyl-N-[2-methyl-3- (3-{[(3R)-1-(prop-2- enoyl)pyrrolidin-3- yl]amino}-1H- pyrazolo[3,4-b]pyridin-4- yl)phenyl]-1,2,4- oxadiazole-3-carboxamide |
| 268 | 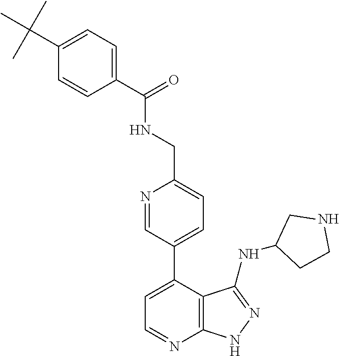 [SMILES: CC(C)(C)c1ccc(C(=O)NCc2ccc(-c3ccnc4[nH]nc(NC5CCNC5)c34)cn2)cc1] | 4-tert-butyl-N-[(5-{3- [(pyrrolidin-3-yl)amino]- 1H-pyrazolo[3,4-b]pyridin- 4-yl}pyridin-2- yl)methyl]benzamide |
| 269 | 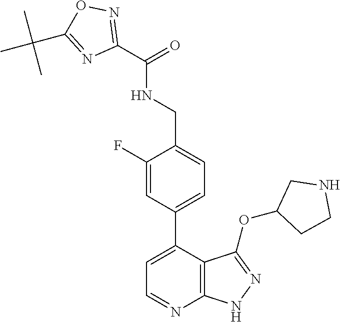 [SMILES: CC(C)(C)c1nc(C(=O)NCc2ccc(-c3ccnc4[nH]nc(OC5CCNC5)c34)cc2F)no1] | 5-tert-butyl-N-[(2-fluoro-4- {3-[(pyrrolidin-3-yl)oxy]- 1H-pyrazolo[3,4-b]pyridin- 4-yl}phenyl)methyl]-1,2,4- oxadiazole-3-carboxamide |
| 270 | 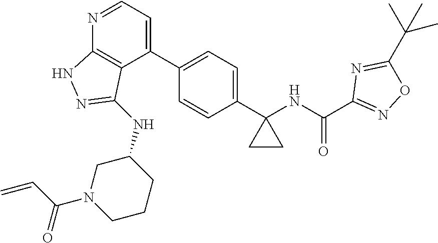 [SMILES: C=CC(=O)N1CCC[C@@H](Nc2n[nH]c3nccc(-c4ccc(C5(NC(=O)c6noc(C(C)(C)C)n6)CC5)cc4)c23)C1] | 5-tert-butyl-N-{1-[4-(3- {[(3R)-1-(prop-2- enoyl)piperidin-3- yl]amino}-1H- pyrazolo[3,4-b]pyridin-4- yl)phenyl]cyclopropyl}- 1,2,4-oxadiazole-3- carboxamide |
| 271 | 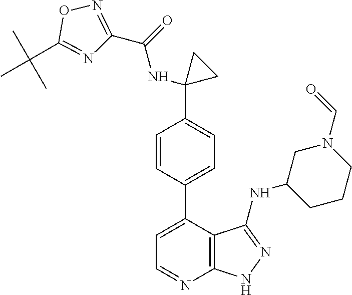 [SMILES: CC(C)(C)c1nc(C(=O)NC2(c3ccc(-c4ccnc5[nH]nc(NC6CCCN(C=O)C6)c45)cc3)CC2)no1] | 5-tert-butyl-N-[1-(4-{3-[(1- formylpiperidin-3- yl)amino]-1H- pyrazolo[3,4-b]pyridin-4- yl}phenyl)cyclopropyl]- 1,2,4-oxadiazole-3- carboxamide |
| 272 | 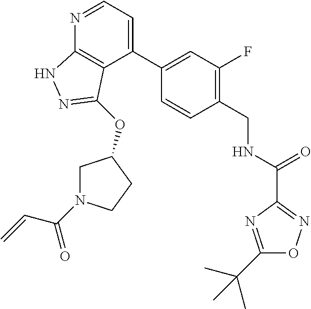 [SMILES: C=CC(=O)N1CC[C@@H](Oc2n[nH]c3nccc(-c4ccc(CNC(=O)c5noc(C(C)(C)C)n5)c(F)c4)c23)C1] | 5-tert-butyl-N-{[2-fluoro-4- (3-{[(3R)-1-(prop-2- enoyl)pyrrolidin-3-yl]oxy}- 1H-pyrazolo[3,4-b]pyridin- 4-yl)phenyl]methyl}-1,2,4- oxadiazole-3-carboxamide |
| 273 | 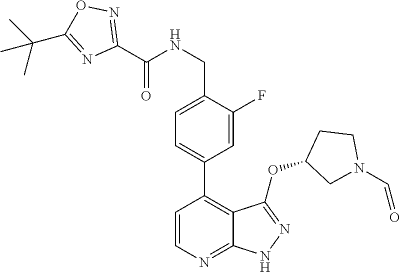 [SMILES: CC(C)(C)c1nc(C(=O)NCc2ccc(-c3ccnc4[nH]nc(O[C@@H]5CCN(C=O)C5)c34)cc2F)no1] | 5-tert-butyl-N-[(2-fluoro-4- {3-[(1-formylpyrrolidin-3- yl)oxy]-1H-pyrazolo[3,4- b]pyridin-4- yl}phenyl)methyl]-1,2,4- oxadiazole-3-carboxamide |
| 274 | 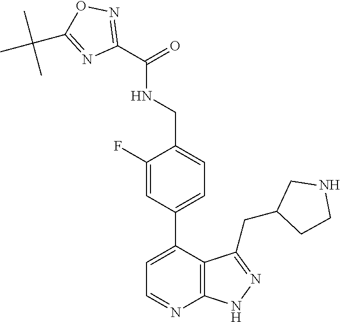 [SMILES: CC(C)(C)c1nc(C(=O)NCc2ccc(-c3ccnc4[nH]nc(CC5CCNC5)c34)cc2F)no1] | (R)-5-tert-butyl-N-[(2- fluoro-4-{3-[(pyrrolidin-3- yl)methyl]-1H- pyrazolo[3,4-b]pyridin-4- yl}phenyl)methyl]-1,2,4- oxadiazole-3-carboxamide |
| 275 | 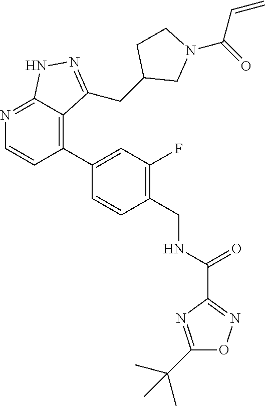 [SMILES: C=CC(=O)N1CCC(Cc2n[nH]c3nccc(-c4ccc(CNC(=O)c5noc(C(C)(C)C)n5)c(F)c4)c23)C1] | 5-tert-butyl-N-{[2-fluoro-4- (3-{[1-(prop-2- enoyl)pyrrolidin-3- yl]methyl}-1H- pyrazolo[3,4-b]pyridin-4- yl)phenyl]methyl}-1,2,4- oxadiazole-3-carboxamide |
| 278 | 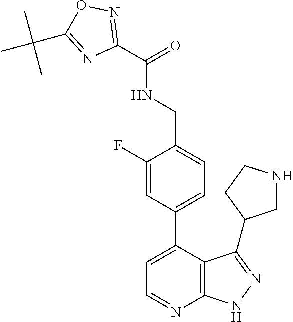 [SMILES: CC(C)(C)c1nc(C(=O)NCc2ccc(-c3ccnc4[nH]nc(C5CCNC5)c34)cc2F)no1] | 5-tert-butyl-N-({2-fluoro-4- [3-(pyrrolidin-3-yl)-1H- pyrazolo[3,4-b]pyridin-4- yl]phenyl}methyl)-1,2,4- oxadiazole-3-carboxamide |
| 279 | 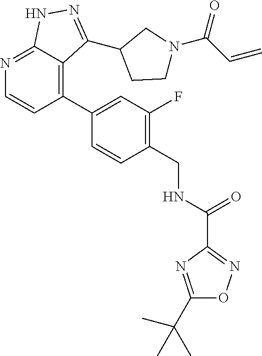 [SMILES: C=CC(=O)N1CCC(c2n[nH]c3nccc(-c4ccc(CNC(=O)c5noc(C(C)(C)C)n5)c(F)c4)c23)C1] | 5-tert-butyl-N-[(2-fluoro-4- {3-[1-(prop-2- enoyl)pyrrolidin-3-yl]-1H- pyrazolo[3,4-b]pyridin-4- yl}phenyl)methyl]-1,2,4- oxadiazole-3-carboxamide |
| 280 | 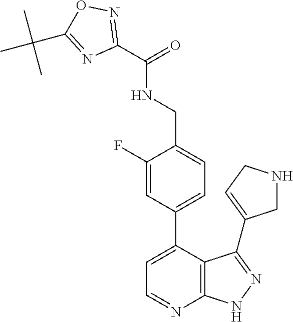 [SMILES: CC(C)(C)c1nc(C(=O)NCc2ccc(-c3ccnc4[nH]nc(C5=CCNC5)c34)cc2F)no1] | 5-tert-butyl-N-({4-[3-(2,5- dihydro-1H-pyrrol-3-yl)- 1H-pyrazolo[3,4-b]pyridin- 4-yl]-2- fluorophenyl}methyl)- 1,2,4-oxadiazole-3- carboxamide |
| 281 | 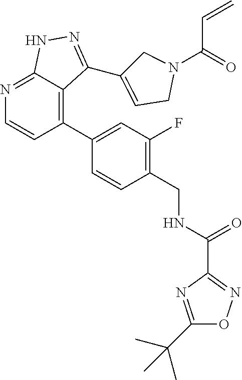 [SMILES: C=CC(=O)N1CC=C(c2n[nH]c3nccc(-c4ccc(CNC(=O)c5noc(C(C)(C)C)n5)c(F)c4)c23)C1] | 5-tert-butyl-N-[(2-fluoro-4- {3-[1-(prop-2-enoyl)-2,5- dihydro-1H-pyrrol-3-yl]- 1H-pyrazolo[3,4-b]pyridin- 4-yl}phenyl)methyl]-1,2,4- oxadiazole-3-carboxamide |
| 282 | 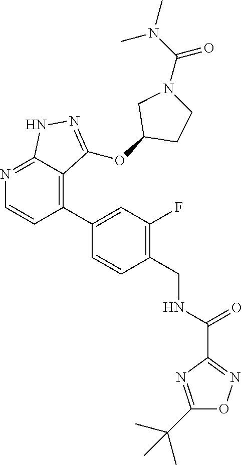 [SMILES: CN(C)C(=O)N1CC[C@@H](Oc2n[nH]c3nccc(-c4ccc(CNC(=O)c5noc(C(C)(C)C)n5)c(F)c4)c23)C1] | 5-tert-butyl-N-{[4-(3- {[(3R)-1- (dimethylcarbamoyl)pyrroli- din-3-yl]oxy}-1H- pyrazolo[3,4-b]pyridin-4- yl)-2- fluorophenyl]methyl}- 1,2,4-oxadiazole-3- carboxamide |
| 283 | 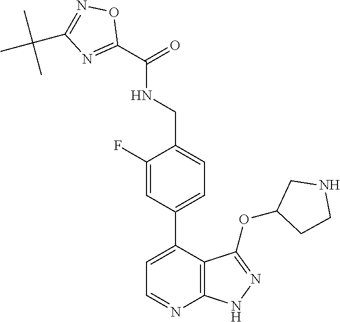 [SMILES: CC(C)(C)c1noc(C(=O)NCc2ccc(-c3ccnc4[nH]nc(OC5CCNC5)c34)cc2F)n1] | 3-tert-butyl-N-[(2-fluoro-4- {3-[(pyrrolidin-3-yl)oxy]- 1H-pyrazolo[3,4-b]pyridin- 4-yl}phenyl)methyl]-1,2,4- oxadiazole-5-carboxamide |
| 284 | 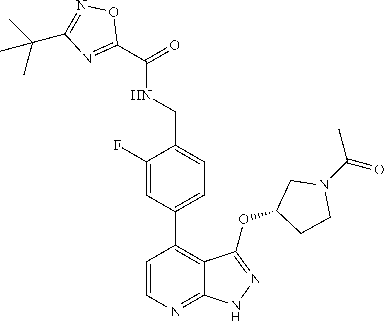 [SMILES: CC(=O)N1CC[C@H](Oc2n[nH]c3nccc(-c4ccc(CNC(=O)c5nc(C(C)(C)C)no5)c(F)c4)c23)C1] | (S)-N-[(4-{3-[(1- acetylpyrrolidin-3-yl)oxy]- 1H-pyrazolo[3,4-b]pyridin- 4-yl}-2- fluorophenyl)methyl]-3- tert-butyl-1,2,4-oxadiazole- 5-carboxamide |
| 285 | 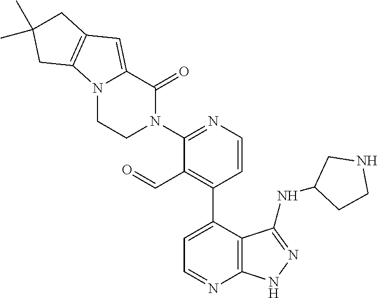 [SMILES: CC1(C)Cc2cc3n(c2C1)CCN(c1nccc(-c2ccnc4[nH]nc(NC5CCNC5)c24)c1C=O)C3=O] | 2-(7,7-dimethyl-1-oxo- 1,3,4,6,7,8-hexahydro-2H- cyclopenta[4,5]pyrrolo[1,2- a]pyrazin-2-yl)-4-{3- [(pyrrolidin-3-yl)amino]- 1H-pyrazolo[3,4-b]pyridin- 4-yl}pyridine-3- carbaldehyde |
| 286 | 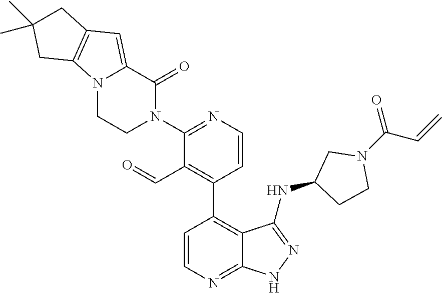 [SMILES: C=CC(=O)N1CC[C@@H](Nc2n[nH]c3nccc(-c4ccnc(N5CCn6c(cc7c6CC(C)(C)C7)C5=O)c4C=O)c23)C1] | (R)-2-(7,7-dimethyl-1-oxo- 1,3,4,6,7,8-hexahydro-2H- cyclopenta[4,5]pyrrolo[1,2- a]pyrazin-2-yl)-4-(3-{[1- (prop-2-enoyl)pyrrolidin-3- yl]amino}-1H- pyrazolo[3,4-b]pyridin-4- yl)pyridine-3-carbaldehyde |
| 287 | 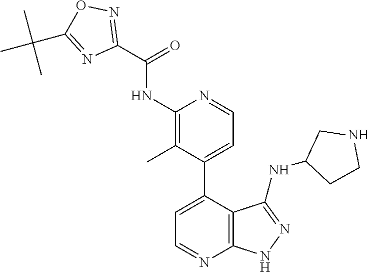 [SMILES: Cc1c(-c2ccnc3[nH]nc(NC4CCNC4)c23)ccnc1NC(=O)c1noc(C(C)(C)C)n1] | 5-tert-butyl-N-(3-methyl-4- {3-[(pyrrolidin-3- yl)amino]-1H- pyrazolo[3,4-b]pyridin-4- yl}pyridin-2-yl)-1,2,4- oxadiazole-3-carboxamide |
| 288 | 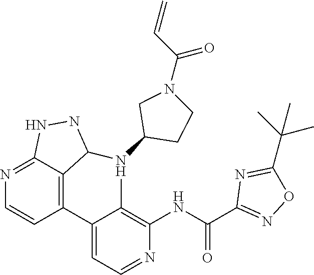 [SMILES: C=CC(=O)N1CC[C@@H](N2c3c(ccnc3NC(=O)c3noc(C(C)(C)C)n3)-c3ccnc4c3C2NN4)C1] | 5-tert-butyl-N-[3-methyl-4- (3-{[(3R)-1-(prop-2- enoyl)pyrrolidin-3- yl]amino}-1H- pyrazolo[3,4-b]pyridin-4- yl)pyridin-2-yl]-1,2,4- oxadiazole-3-carboxamide |
| 289 | 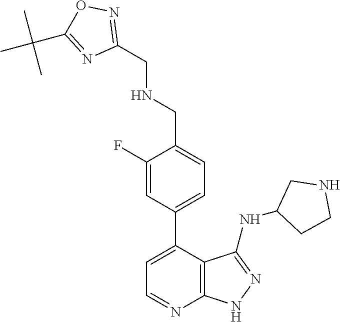 [SMILES: CC(C)(C)c1nc(CNCc2ccc(-c3ccnc4[nH]nc(NC5CCNC5)c34)cc2F)no1] | 4-[4-({[(5-tert-butyl-1,2,4- oxadiazol-3- yl)methyl]amino}methyl)- 3-fluorophenyl]-N- (pyrrolidin-3-yl)-1H- pyrazolo[3,4-b]pyridin-3- amine |
| 290 | 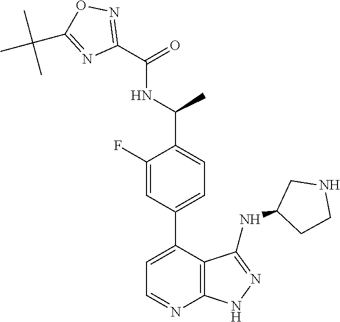 [SMILES: C[C@H](NC(=O)c1noc(C(C)(C)C)n1)c1ccc(-c2ccnc3[nH]nc(N[C@@H]4CCNC4)c23)cc1F] | 5-tert-butyl-N-{(1S)-1-[2- fluoro-4-(3-{[(3R)- pyrrolidin-3-yl]amino}-1H- pyrazolo[3,4-b]pyridin-4- yl)phenyl]ethyl}-1,2,4- oxadiazole-3-carboxamide |
| 291 | 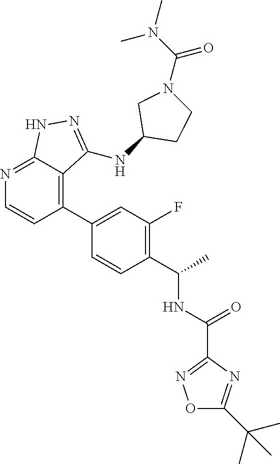 [SMILES: C[C@H](NC(=O)c1noc(C(C)(C)C)n1)c1ccc(-c2ccnc3[nH]nc(N[C@@H]4CCN(C(=O)N(C)C)C4)c23)cc1F] | 5-tert-butyl-N-{(1S)-1-[4- (3-{[(3R)-1- (dimethylcarbamoyl)pyrroli- din-3-yl]amino}-1H- pyrazolo[3,4-b]pyridin-4- yl)-2-fluorophenyl]ethyl}- 1,2,4-oxadiazole-3- carboxamide |
| 292 | 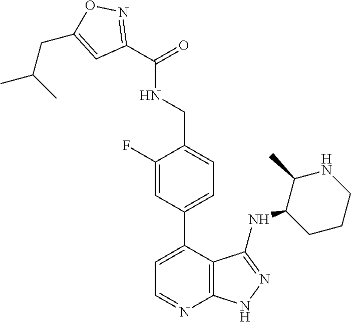 [SMILES: CC(C)Cc1cc(C(=O)NCc2ccc(-c3ccnc4[nH]nc(N[C@@H]5CCCN[C@@H]5C)c34)cc2F)no1] | N-{[2-fluoro-4-(3- {[(2R,3R)-2- methylpiperidin-3- yl]amino}-1H- pyrazolo[3,4-b]pyridin-4- yl)phenyl]methyl}-5-(2- methylpropyl)-1,2-oxazole- 3-carboxamide |
| 293 | 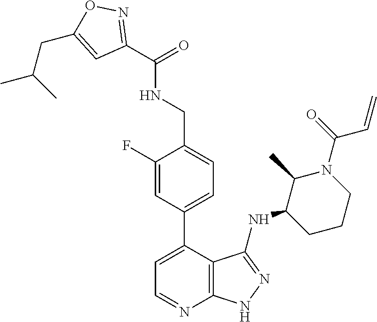 [SMILES: C=CC(=O)N1CCC[C@@H](Nc2n[nH]c3nccc(-c4ccc(CNC(=O)c5cc(CC(C)C)on5)c(F)c4)c23)[C@H]1C] | N-{[2-fluoro-4-(3- {[(2R,3R)-2-methyl-1- (prop-2-enoyl)piperidin-3- yl]amino}-1H- pyrazolo[3,4-b]pyridin-4- yl)phenyl]methyl}-5-(2- methylpropyl)-1,2-oxazole- 3-carboxamide |
| 294 | 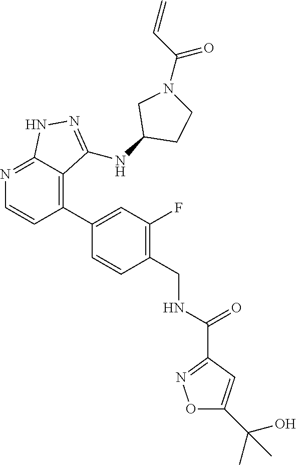 [SMILES: C=CC(=O)N1CC[C@@H](Nc2n[nH]c3nccc(-c4ccc(CNC(=O)c5cc(C(C)(C)O)on5)c(F)c4)c23)C1] | N-{[2-fluoro-4-(3-{[(3R)-1- (prop-2-enoyl)pyrrolidin-3- yl]amino}-1H- pyrazolo[3,4-b]pyridin-4- yl)phenyl]methyl}-5-(2- hydroxypropan-2-yl)-1,2- oxazole-3-carboxamide |
| 295 | 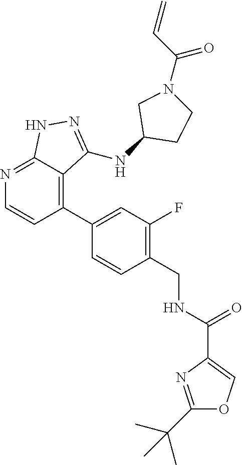 [SMILES: C=CC(=O)N1CC[C@@H](Nc2n[nH]c3nccc(-c4ccc(CNC(=O)c5coc(C(C)(C)C)n5)c(F)c4)c23)C1] | 2-tert-butyl-N-{[2-fluoro-4- (3-{[(3R)-1-(prop-2- enoyl)pyrrolidin-3- yl]amino}-1H- pyrazolo[3,4-b]pyridin-4- yl)phenyl]methyl}-1,3- oxazole-4-carboxamide |
| 296 | 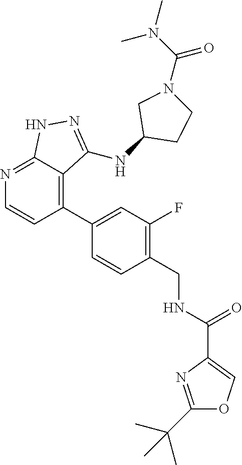 [SMILES: CN(C)C(=O)N1CC[C@@H](Nc2n[nH]c3nccc(-c4ccc(CNC(=O)c5coc(C(C)(C)C)n5)c(F)c4)c23)C1] | 2-tert-butyl-N-{[4-(3- {[(3R)-1- (dimethylcarbamoyl)pyrroli- din-3-yl]amino}-1H- pyrazolo[3,4-b]pyridin-4- yl)-2- fluorophenyl]methyl}-1,3- oxazole-4-carboxamide |
| 297 | 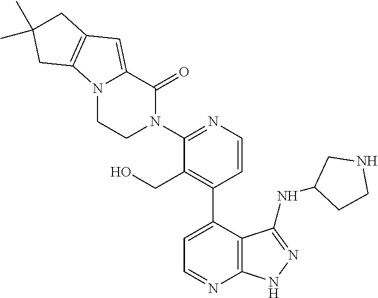 [SMILES: CC1(C)Cc2cc3n(c2C1)CCN(c1nccc(-c2ccnc4[nH]nc(NC5CCNC5)c24)c1CO)C3=O] | 2-[3-(hydroxymethyl)-4- {3-[(pyrrolidin-3- yl)amino]-1H- pyrazolo[3,4-b]pyridin-4- yl}pyridin-2-yl]-7,7- dimethyl-3,4,7,8- tetrahydro-2H- cyclopenta[4,5]pyrrolo[1,2- a]pyrazin-1(6H)-one |
| 298 | 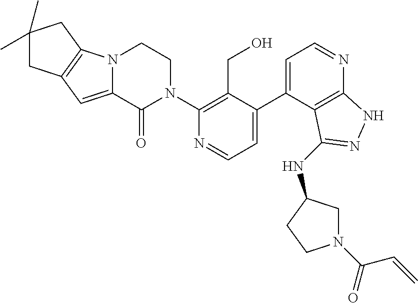 [SMILES: C=CC(=O)N1CC[C@@H](Nc2n[nH]c3nccc(-c4ccnc(N5CCn6c(cc7c6CC(C)(C)C7)C5=O)c4CO)c23)C1] | 2-[3-(hydroxymethyl)-4-(3- {[(3R)-1-(prop-2- enoyl)pyrrolidin-3- yl]amino}-1H- pyrazolo[3,4-b]pyridin-4- yl)pyridin-2-yl]-7,7- dimethyl-3,4,7,8- tetrahydro-2H- cyclopenta[4,5]pyrrolo[1,2- a]pyrazin-1(6H)-one |
| 299 | 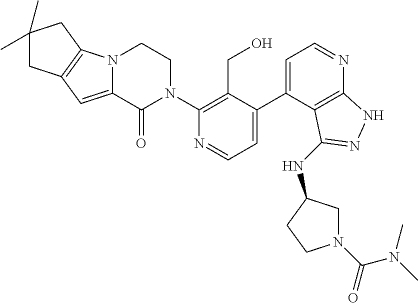 | (3R)-3-({4-[2-(7,7- dimethyl-1-oxo-1,3,4,6,7,8- hexahydro-2H- cyclopenta[4,5]pyrrolo[1,2- a]pyrazin-2-yl)-3- (hydroxymethyl)pyridin-4- yl]-1H-pyrazolo[3,4- b]pyridin-3-yl}amino)-N,N- dimethylpyrrolidine-1- carboxamide |
| 300 | 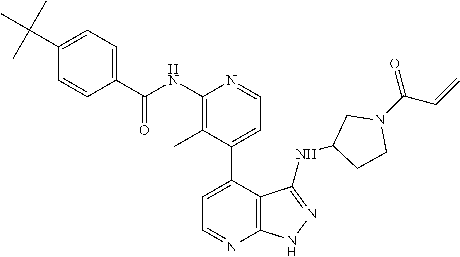 [SMILES: C=CC(=O)N1CCC(Nc2n[nH]c3nccc(-c4ccnc(NC(=O)c5ccc(C(C)(C)C)cc5)c4C)c23)C1] | 4-tert-butyl-N-[3-methyl-4- (3-{[1-(prop-2- enoyl)pyrrolidin-3- yl]amino}-1H- pyrazolo[3,4-b]pyridin-4- yl)pyridin-2-yl]benzamide |
| 301 | 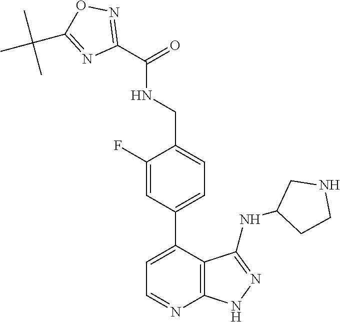 [SMILES: CC(C)(C)c1nc(C(=O)NCc2ccc(-c3ccnc4[nH]nc(NC5CCNC5)c34)cc2F)no1] | N-[(5-tert-butyl-1,2,4- oxadiazol-3-yl)methyl]-2- fluoro-4-{3-[(pyrrolidin-3- yl)amino]-1H- pyrazolo[3,4-b]pyridin-4- yl}benzamide |
| 302 | 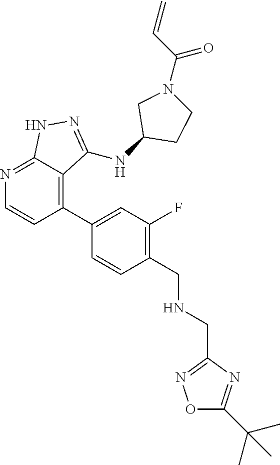 [SMILES: C=CC(=O)N1CC[C@@H](Nc2n[nH]c3nccc(-c4ccc(CNCc5noc(C(C)(C)C)n5)c(F)c4)c23)C1] | N-[(5-tert-butyl-1,2,4- oxadiazol-3-yl)methyl]-2- fluoro-4-(3-{[(3R)-1-(prop- 2-enoyl)pyrrolidin-3- yl]amino}-1H- pyrazolo[3,4-b]pyridin-4- yl)benzamide |
| 303 | 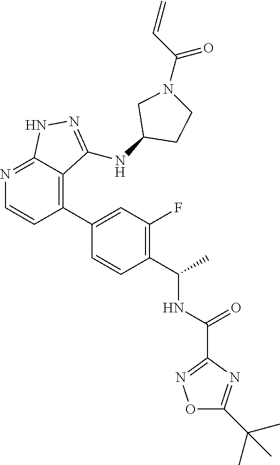 [SMILES: C=CC(=O)N1CC[C@@H](Nc2n[nH]c3nccc(-c4ccc([C@H](C)NC(=O)c5noc(C(C)(C)C)n5)c(F)c4)c23)C1] | 5-tert-butyl-N-{(1S)-1-[2- fluoro-4-(3-{[(3R)-1-(prop- 2-enoyl)pyrrolidin-3- yl]amino}-1H- pyrazolo[3,4-b]pyridin-4- yl)phenyl]ethyl}-1,2,4- oxadiazole-3-carboxamide |
| 304 | 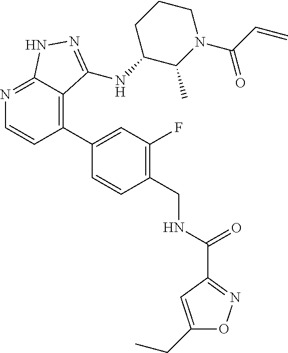 [SMILES: C=CC(=O)N1CCC[C@@H](Nc2n[nH]c3nccc(-c4ccc(CNC(=O)c5cc(CC)on5)c(F)c4)c23)[C@H]1C] | 5-ethyl-N-{[2-fluoro-4-(3- {[(2R,3R)-2-methyl-1- (prop-2-enoyl)piperidin-3- yl]amino}-1H- pyrazolo[3,4-b]pyridin-4- yl)phenyl]methyl}-1,2- oxazole-3-carboxamide |
| 305 | 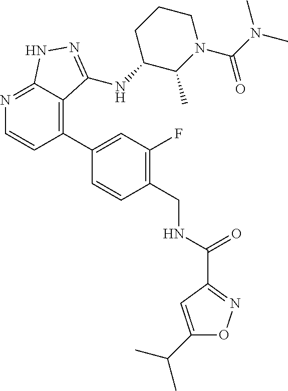 [SMILES: CC(C)c1cc(C(=O)NCc2ccc(-c3ccnc4[nH]nc(N[C@@H]5CCCN(C(=O)N(C)C)[C@@H]5C)c34)cc2F)no1] | (2R,3R)-3-({4-[3-fluoro-4- ({[5-(propan-2-yl)-1,2- oxazole-3- carbonyl]amino}methyl) phenyl]-1H-pyrazolo[3,4- b]pyridin-3-yl}amino)- N,N,2-trimethylpiperidine- 1-carboxamide |
| 306 | 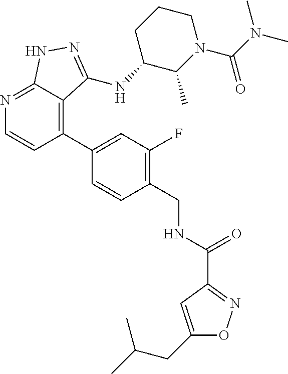 [SMILES: CC(C)Cc1cc(C(=O)NCc2ccc(-c3ccnc4[nH]nc(N[C@@H]5CCCN(C(=O)N(C)C)[C@@H]5C)c34)cc2F)no1] | (2R,3R)-3-({4-[3-fluoro-4- ({[5-(2-methylpropyl)-1,2- oxazole-3- carbonyl]amino}methyl) phenyl]-1H-pyrazolo[3,4- b]pyridin-3-yl}amino)- N,N,2-trimethylpiperidine- 1-carboxamide |
| 307 | 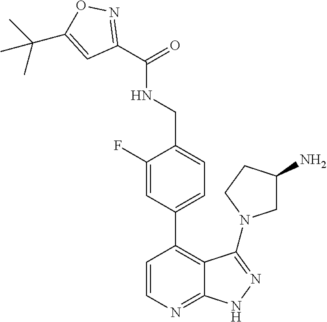 [SMILES: CC(C)(C)c1cc(C(=O)NCc2ccc(-c3ccnc4[nH]nc(N5CC[C@@H](N)C5)c34)cc2F)no1] | (R)-N-({4-[3-(3- aminopyrrolidin-1-yl)-1H- pyrazolo[3,4-b]pyridin-4- yl]-2- fluorophenyl}methyl)-5- tert-butyl-1,2-oxazole-3- carboxamide |
| 308 | 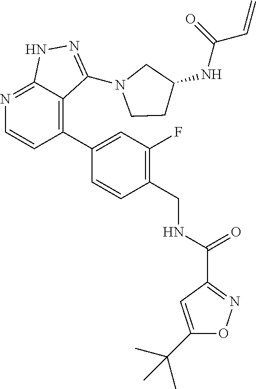 [SMILES: C=CC(=O)N[C@@H]1CCN(c2n[nH]c3nccc(-c4ccc(CNC(=O)c5cc(C(C)(C)C)on5)c(F)c4)c23)C1] | 5-tert-butyl-N-{[2-fluoro-4- (3-{(3R)-3-[(prop-2- enoyl)amino]pyrrolidin-1- yl}-1H-pyrazolo[3,4- b]pyridin-4- yl)phenyl]methyl}-1,2- oxazole-3-carboxamide |
| 309 | 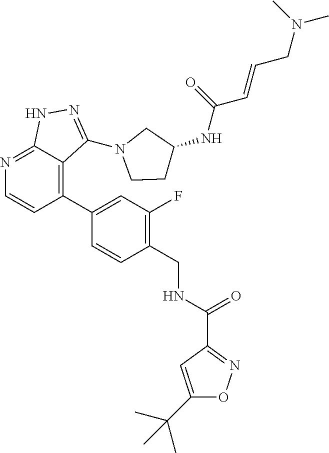 [SMILES: CN(C)C/C=C/C(=O)N[C@@H]1CCN(c2n[nH]c3nccc(-c4ccc(CNC(=O)c5cc(C(C)(C)C)on5)c(F)c4)c23)C1] | 5-tert-butyl-N-[(4-{3-[(3R)- 3-{[(2E)-4- (dimethylamino)but-2- enoyl]amino}pyrrolidin-1- yl]-1H-pyrazolo[3,4- b]pyridin-4-yl}-2- fluorophenyl)methyl]-1,2- oxazole-3-carboxamide |
| 310 | 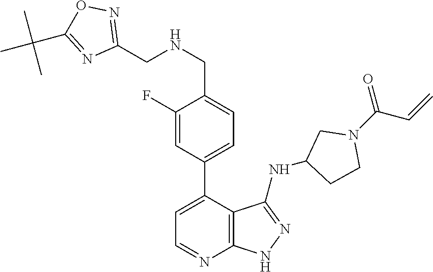 [SMILES: C=CC(=O)N1CCC(Nc2n[nH]c3nccc(-c4ccc(CNCc5noc(C(C)(C)C)n5)c(F)c4)c23)C1] | 1-[3-({4-[4-({[(5-tert-butyl- 1,2,4-oxadiazol-3- yl)methyl]amino}methyl)- 3-fluorophenyl]-1H- pyrazolo[3,4-b]pyridin-3- yl}amino)pyrrolidin-1- yl]prop-2-en-1-one |
| 312 | 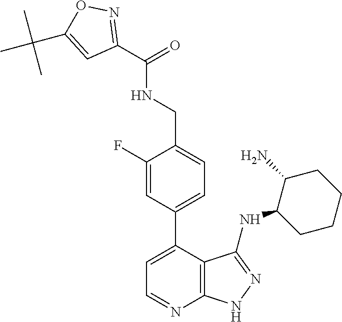 [SMILES: CC(C)(C)c1cc(C(=O)NCc2ccc(-c3ccnc4[nH]nc(N[C@@H]5CCCC[C@H]5N)c34)cc2F)no1] | N-{[4-(3-{[(1R,2R)-2- aminocyclohexyl]amino}- 1H-pyrazolo[3,4-b]pyridin- 4-yl)-2- fluorophenyl]methyl}-5- tert-butyl-1,2-oxazole-3- carboxamide |
| 313 | 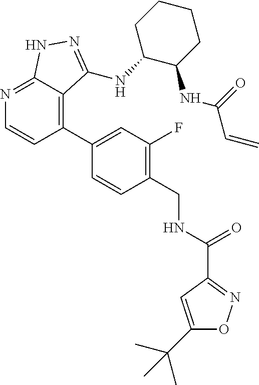 [SMILES: C=CC(=O)N[C@@H]1CCCC[C@H]1Nc1n[nH]c2nccc(-c3ccc(CNC(=O)c4cc(C(C)(C)C)on4)c(F)c3)c12] | 5-tert-butyl-N-({2-fluoro-4- [3-({(1R,2R)-2-[(prop-2- enoyl)amino]cyclohexyl} amino)-1H-pyrazolo[3,4- b]pyridin-4- yl]phenyl}methyl)-1,2- oxazole-3-carboxamide |
| 315 | 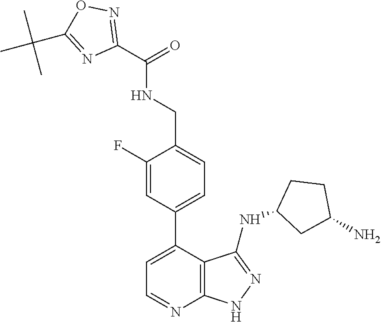 [SMILES: CC(C)(C)c1nc(C(=O)NCc2ccc(-c3ccnc4[nH]nc(N[C@@H]5CC[C@H](N)C5)c34)cc2F)no1] | N-{[4-(3-{[(1R,3S)-3- aminocyclopentyl]amino}- 1H-pyrazolo[3,4-b]pyridin- 4-yl)-2- fluorophenyl]methyl}-5- tert-butyl-1,2,4-oxadiazole- 3-carboxamide |
| 316 | 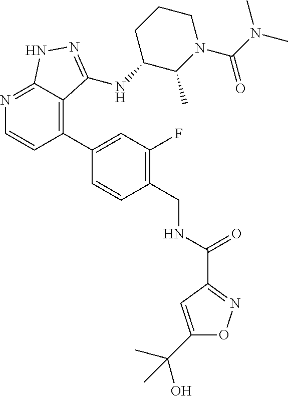 [SMILES: C[C@@H]1[C@H](Nc2n[nH]c3nccc(-c4ccc(CNC(=O)c5cc(C(C)(C)O)on5)c(F)c4)c23)CCCN1C(=O)N(C)C] | (2R,3R)-3-({4-[3-fluoro-4- ({[5-(2-hydroxypropan-2- yl)-1,2-oxazole-3- carbonyl]amino}methyl) phenyl]-1H-pyrazolo[3,4- b]pyridin-3-yl}amino)- N,N,2-trimethylpiperidine- 1-carboxamide |
| 317 | 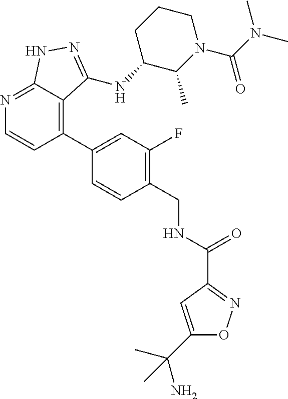 [SMILES: C[C@@H]1[C@H](Nc2n[nH]c3nccc(-c4ccc(CNC(=O)c5cc(C(C)(C)N)on5)c(F)c4)c23)CCCN1C(=O)N(C)C] | (2R,3R)-3-({4-[4-({[5-(2- aminopropan-2-yl)-1,2- oxazole-3- carbonyl]amino}methyl)-3- fluorophenyl]-1H- pyrazolo[3,4-b]pyridin-3- yl}amino)-N,N,2- trimethylpiperidine-1- carboxamide |
| 318 | 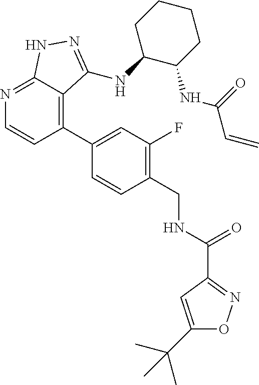 [SMILES: C=CC(=O)N[C@H]1CCCC[C@@H]1Nc1n[nH]c2nccc(-c3ccc(CNC(=O)c4cc(C(C)(C)C)on4)c(F)c3)c12] | 5-tert-butyl-N-({2-fluoro-4- [3-({(1S,2S)-2-[(prop-2- enoyl)amino]cyclohexyl} amino)-1H-pyrazolo[3,4- b]pyridin-4- yl]phenyl}methyl)-1,2- oxazole-3-carboxamide |
| 320 | 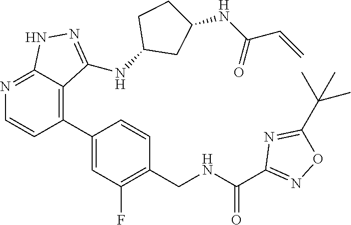 [SMILES: C=CC(=O)N[C@H]1CC[C@@H](Nc2n[nH]c3nccc(-c4ccc(CNC(=O)c5noc(C(C)(C)C)n5)c(F)c4)c23)C1] | 5-tert-butyl-N-({2-fluoro-4- [3-({(1R,3S)-3-[(prop-2- enoyl)amino]cyclopentyl} amino)-1H-pyrazolo[3,4- b]pyridin-4- yl]phenyl}methyl)-1,2,4- oxadiazole-3-carboxamide |
| 322 | 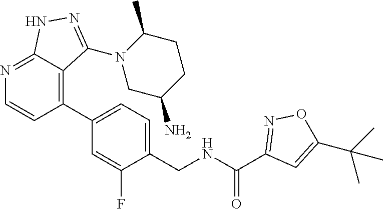 [SMILES: C[C@H]1CC[C@@H](N)CN1c1n[nH]c2nccc(-c3ccc(CNC(=O)c4cc(C(C)(C)C)on4)c(F)c3)c12] | N-[(4-{3-[(2S,5R)-5-amino- 2-methylpiperidin-1-yl]- 1H-pyrazolo[3,4-b]pyridin- 4-yl}-2- fluorophenyl)methyl]-5- tert-butyl-1,2-oxazole-3- carboxamide |
| 323 | 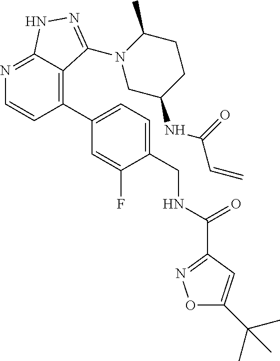 [SMILES: C=CC(=O)N[C@@H]1CC[C@H](C)N(c2n[nH]c3nccc(-c4ccc(CNC(=O)c5cc(C(C)(C)C)on5)c(F)c4)c23)C1] | 5-tert-butyl-N-{[2-fluoro-4- (3-{(2S,5R)-2-methyl-5- [(prop-2- enoyl)amino]piperidin-1- yl}-1H-pyrazolo[3,4- b]pyridin-4- yl)phenyl]methyl}-1,2- oxazole-3-carboxamide |
| 325 | 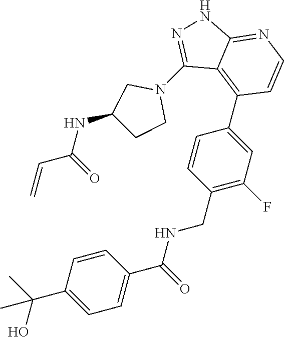 [SMILES: C=CC(=O)N[C@@H]1CCN(c2n[nH]c3nccc(-c4ccc(CNC(=O)c5ccc(C(C)(C)O)cc5)c(F)c4)c23)C1] | N-{[2-fluoro-4-(3-{(3R)-3- [(prop-2- enoyl)amino]pyrrolidin-1- yl}-1H-pyrazolo[3,4- b]pyridin-4- yl)phenyl]methyl}-4-(2- hydroxypropan-2- yl)benzamide |
| 328 | 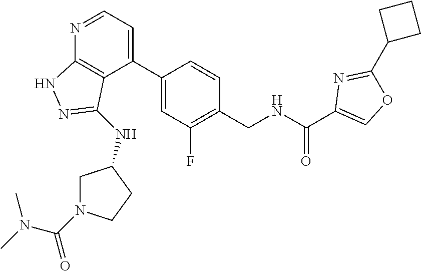 [SMILES: CN(C)C(=O)N1CC[C@@H](Nc2n[nH]c3nccc(-c4ccc(CNC(=O)c5coc(C6CCC6)n5)c(F)c4)c23)C1] | 2-cyclobutyl-N-{[4-(3- {[(3R)-1- (dimethylcarbamoyl)pyrroli- din-3-yl]amino}-1H- pyrazolo[3,4-b]pyridin-4- yl)-2- fluorophenyl]methyl}-1,3- oxazole-4-carboxamide |
| 329 | 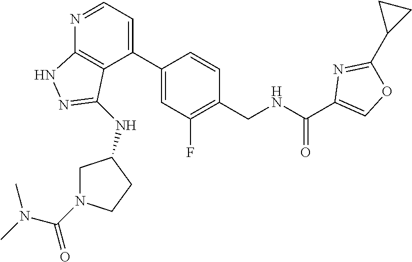 [SMILES: CN(C)C(=O)N1CC[C@@H](Nc2n[nH]c3nccc(-c4ccc(CNC(=O)c5coc(C6CC6)n5)c(F)c4)c23)C1] | 2-cyclopropyl-N-{[4-(3- {[(3R)-1- (dimethylcarbamoyl)pyrroli- din-3-yl]amino}-1H- pyrazolo[3,4-b]pyridin-4- yl)-2- fluorophenyl]methyl}-1,3- oxazole-4-carboxamide |
| 330 | 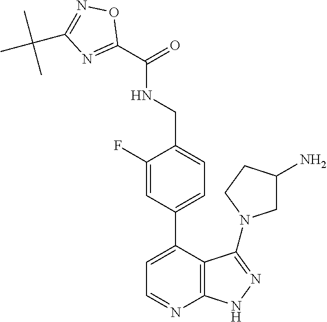 [SMILES: CC(C)(C)c1noc(C(=O)NCc2ccc(-c3ccnc4[nH]nc(N5CCC(N)C5)c34)cc2F)n1] | N-({4-[3-(3- aminopyrrolidin-1-yl)-1H- pyrazolo[3,4-b]pyridin-4- yl]-2- fluorophenyl}methyl)-3- tert-butyl-1,2,4-oxadiazole- 5-carboxamide |
| 331 | 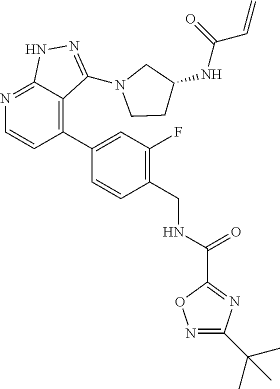 [SMILES: C=CC(=O)N[C@@H]1CCN(c2n[nH]c3nccc(-c4ccc(CNC(=O)c5nc(C(C)(C)C)no5)c(F)c4)c23)C1] | 3-tert-butyl-N-{[2-fluoro-4- (3-{(3R)-3-[(prop-2- enoyl)amino]pyrrolidin-1- yl}-1H-pyrazolo[3,4- b]pyridin-4- yl)phenyl]methyl}-1,2,4- oxadiazole-5-carboxamide |
| 333 | 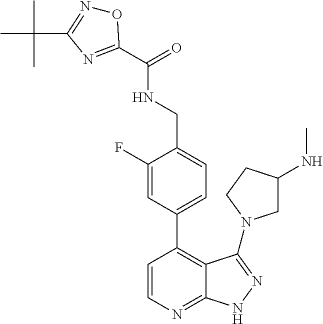 [SMILES: CNC1CCN(c2n[nH]c3nccc(-c4ccc(CNC(=O)c5nc(C(C)(C)C)no5)c(F)c4)c23)C1] | 3-tert-butyl-N-[(2-fluoro-4- {3-[3- (methylamino)pyrrolidin-1- yl]-1H-pyrazolo[3,4- b]pyridin-4- yl}phenyl)methyl]-1,2,4- oxadiazole-5-carboxamide |
| 334 | 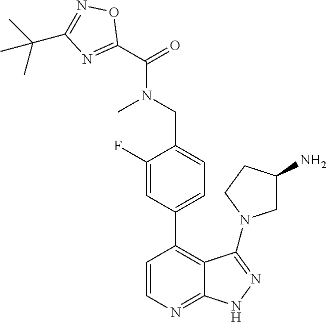 [SMILES: CN(Cc1ccc(-c2ccnc3[nH]nc(N4CC[C@@H](N)C4)c23)cc1F)C(=O)c1nc(C(C)(C)C)no1] | (R)-N-({4-[3-(3- aminopyrrolidin-1-yl)-1H- pyrazolo[3,4-b]pyridin-4- yl]-2- fluorophenyl}methyl)-3- tert-butyl-N-methyl-1,2,4- oxadiazole-5-carboxamide |
| 335 | 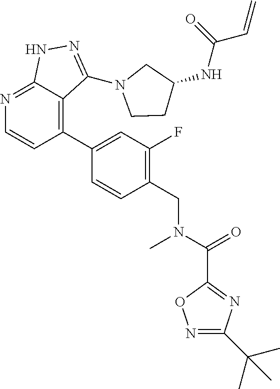 [SMILES: C=CC(=O)N[C@@H]1CCN(c2n[nH]c3nccc(-c4ccc(CN(C)C(=O)c5nc(C(C)(C)C)no5)c(F)c4)c23)C1] | 3-tert-butyl-N-{[2-fluoro-4- (3-{(3R)-3-[(prop-2- enoyl)amino]pyrrolidin-1- yl}-1H-pyrazolo[3,4- b]pyridin-4- yl)phenyl]methyl}-N- methyl-1,2,4-oxadiazole-5- carboxamide |
| 337 | 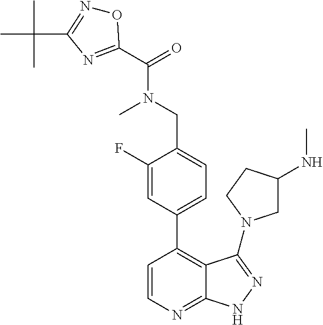 [SMILES: CNC1CCN(c2n[nH]c3nccc(-c4ccc(CN(C)C(=O)c5nc(C(C)(C)C)no5)c(F)c4)c23)C1] | 3-tert-butyl-N-[(2-fluoro-4- {3-[3- (methylamino)pyrrolidin-1- yl]-1H-pyrazolo[3,4- b]pyridin-4- yl}phenyl)methyl]-N- methyl-1,2,4-oxadiazole-5- carboxamide |
| 338 | 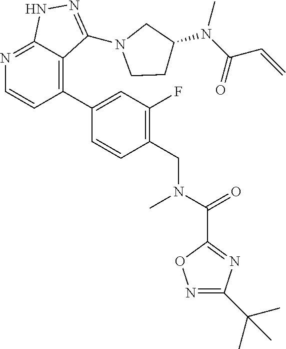 [SMILES: C=CC(=O)N(C)[C@@H]1CCN(c2n[nH]c3nccc(-c4ccc(CN(C)C(=O)c5nc(C(C)(C)C)no5)c(F)c4)c23)C1] | 3-tert-butyl-N-{[2-fluoro-4- (3-{(3R)-3-[methyl(prop-2- enoyl)amino]pyrrolidin-1- yl}-1H-pyrazolo[3,4- b]pyridin-4- yl)phenyl]methyl}-N- methyl-1,2,4-oxadiazole-5- carboxamide |
| 341 | 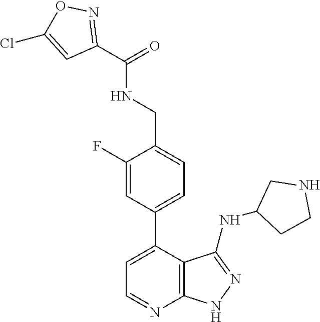 [SMILES: O=C(NCc1ccc(-c2ccnc3[nH]nc(NC4CCNC4)c23)cc1F)c1cc(Cl)on1] | 5-chloro-N-[(2-fluoro-4-{3- [(pyrrolidin-3-yl)amino]- 1H-pyrazolo[3,4-b]pyridin- 4-yl}phenyl)methyl]-1,2- oxazole-3-carboxamide |
| 342 | 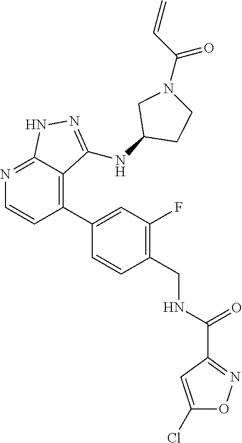 [SMILES: C=CC(=O)N1CC[C@@H](Nc2n[nH]c3nccc(-c4ccc(CNC(=O)c5cc(Cl)on5)c(F)c4)c23)C1] | 5-chloro-N-{[2-fluoro-4-(3- {[(3R)-1-(prop-2- enoyl)pyrrolidin-3- yl]amino}-1H- pyrazolo[3,4-b]pyridin-4- yl)phenyl]methyl}-1,2- oxazole-3-carboxamide |
| 343 | 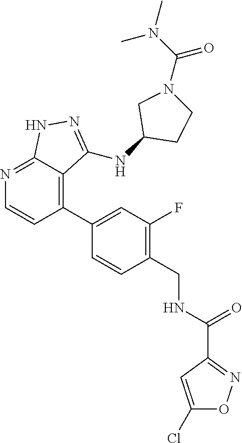 [SMILES: CN(C)C(=O)N1CC[C@@H](Nc2n[nH]c3nccc(-c4ccc(CNC(=O)c5cc(Cl)on5)c(F)c4)c23)C1] | 5-chloro-N-{[4-(3-{[(3R)- 1- (dimethylcarbamoyl)pyrroli- din-3-yl]amino}-1H- pyrazolo[3,4-b]pyridin-4- yl)-2- fluorophenyl]methyl}-1,2- oxazole-3-carboxamide |
| 344 | 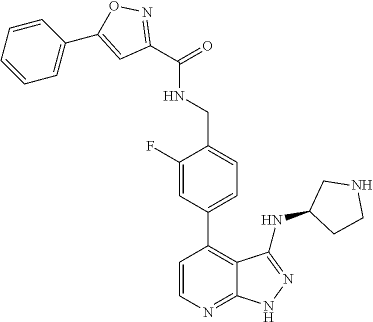 [SMILES: O=C(NCc1ccc(-c2ccnc3[nH]nc(N[C@@H]4CCNC4)c23)cc1F)c1cc(-c2ccccc2)on1] | (R)-N-[(2-fluoro-4-{3- [(pyrrolidin-3-yl)amino]- 1H-pyrazolo[3,4-b]pyridin- 4-yl}phenyl)methyl]-5- phenyl-1,2-oxazole-3- carboxamide |
| 345 | 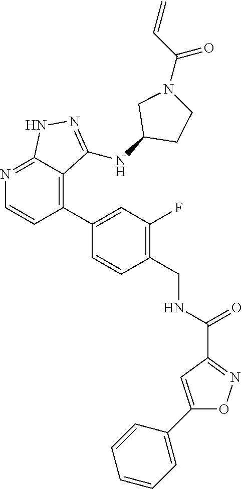 [SMILES: C=CC(=O)N1CC[C@@H](Nc2n[nH]c3nccc(-c4ccc(CNC(=O)c5cc(-c6ccccc6)on5)c(F)c4)c23)C1] | N-{[2-fluoro-4-(3-{[(3R)-1- (prop-2-enoyl)pyrrolidin-3- yl]amino}-1H- pyrazolo[3,4-b]pyridin-4- yl)phenyl]methyl}-5- phenyl-1,2-oxazole-3- carboxamide |
| 346 | 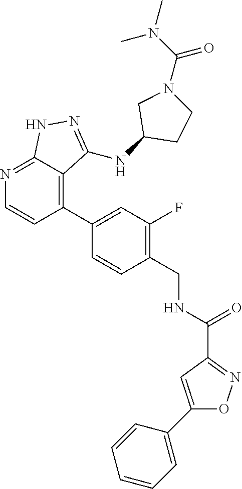 [SMILES: CN(C)C(=O)N1CC[C@@H](Nc2n[nH]c3nccc(-c4ccc(CNC(=O)c5cc(-c6ccccc6)on5)c(F)c4)c23)C1] | N-{[4-(3-{[(3R)-1- (dimethylcarbamoyl)pyrroli- din-3-yl]amino}-1H- pyrazolo[3,4-b]pyridin-4- yl)-2- fluorophenyl]methyl}-5- phenyl-1,2-oxazole-3- carboxamide |
| 347 | 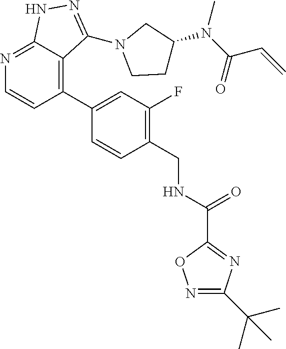 [SMILES: C=CC(=O)N(C)[C@@H]1CCN(c2n[nH]c3nccc(-c4ccc(CNC(=O)c5nc(C(C)(C)C)no5)c(F)c4)c23)C1] | 3-tert-butyl-N-{[2-fluoro-4- (3-{(3R)-3-[methyl(prop-2- enoyl)amino]pyrrolidin-1- yl}-1H-pyrazolo[3,4- b]pyridin-4- yl)phenyl]methyl}-1,2,4- oxadiazole-5-carboxamide |
| 349 | 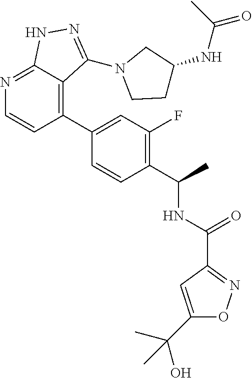 [SMILES: CC(=O)N[C@@H]1CCN(c2n[nH]c3nccc(-c4ccc([C@@H](C)NC(=O)c5cc(C(C)(C)O)on5)c(F)c4)c23)C1] | N-[(1R)-1-(4-{3-[(3R)-3- acetamidopyrrolidin-1-yl]- 1H-pyrazolo[3,4-b]pyridin- 4-yl}-2- fluorophenyl)ethyl]-5-(2- hydroxypropan-2-yl)-1,2- oxazole-3-carboxamide |
| 350 | 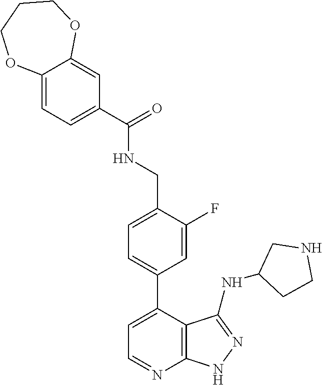 [SMILES: O=C(NCc1ccc(-c2ccnc3[nH]nc(NC4CCNC4)c23)cc1F)c1ccc2c(c1)OCCCO2] | N-[(2-fluoro-4-{3- [(pyrrolidin-3-yl)amino]- 1H-pyrazolo[3,4-b]pyridin- 4-yl}phenyl)methyl]-3,4- dihydro-2H-1,5- benzodioxepine-7- carboxamide |
| 351 | 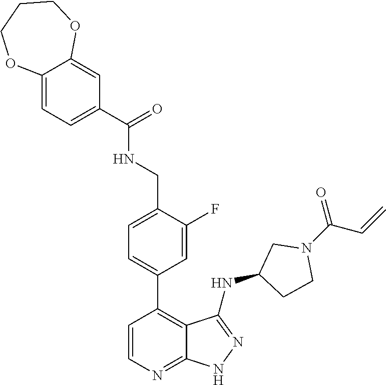 [SMILES: C=CC(=O)N1CC[C@@H](Nc2n[nH]c3nccc(-c4ccc(CNC(=O)c5ccc6c(c5)OCCCO6)c(F)c4)c23)C1] | (R)-N-{[2-fluoro-4-(3-{[1- (prop-2-enoyl)pyrrolidin-3- yl]amino}-1H- pyrazolo[3,4-b]pyridin-4- yl)phenyl]methyl}-3,4- dihydro-2H-1,5- benzodioxepine-7- carboxamide |
| 352 | 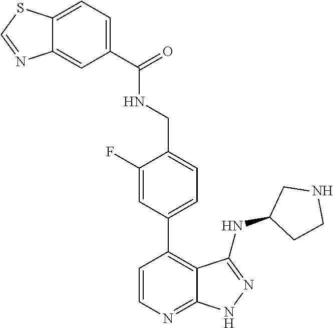 [SMILES: O=C(NCc1ccc(-c2ccnc3[nH]nc(N[C@@H]4CCNC4)c23)cc1F)c1ccc2scnc2c1] | (R)-N-[(2-fluoro-4-{3- [(pyrrolidin-3-yl)amino]- 1H-pyrazolo[3,4-b]pyridin- 4-yl}phenyl)methyl]-1,3- benzothiazole-5- carboxamide |
| 353 | 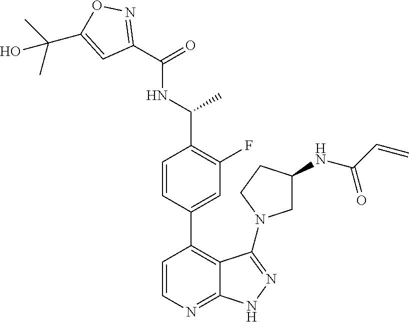 [SMILES: C=CC(=O)N[C@@H]1CCN(c2n[nH]c3nccc(-c4ccc([C@@H](C)NC(=O)c5cc(C(C)(C)O)on5)c(F)c4)c23)C1] | N-{(1R)-1-[2-fluoro-4-(3- {(3R)-3-[(prop-2- enoyl)amino]pyrrolidin-1- yl}-1H-pyrazolo[3,4- b]pyridin-4- yl)phenyl]ethyl}-5-(2- hydroxypropan-2-yl)-1,2- oxazole-3-carboxamide |
| 355 | 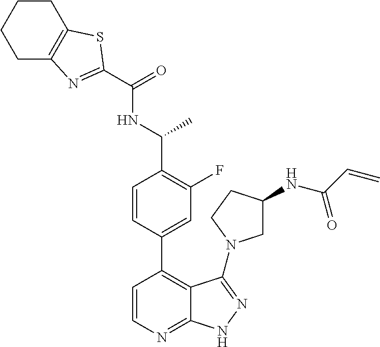 [SMILES: C=CC(=O)N[C@@H]1CCN(c2n[nH]c3nccc(-c4ccc([C@@H](C)NC(=O)c5nc6c(s5)CCCC6)c(F)c4)c23)C1] | N-{[2-fluoro-4-(3-{(3R)-3- [(prop-2- enoyl)amino]pyrrolidin-1- yl}-1H-pyrazolo[3,4- b]pyridin-4- yl)phenyl]methyl}-4,5,6,7- tetrahydro-1,3- benzothiazole-2- carboxamide |
| 356 | 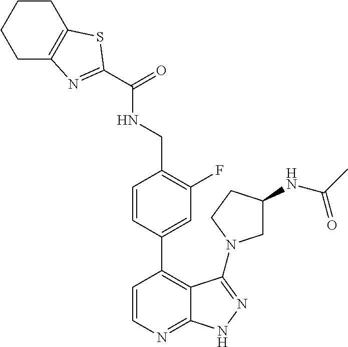 [SMILES: CC(=O)N[C@@H]1CCN(c2n[nH]c3nccc(-c4ccc(CNC(=O)c5nc6c(s5)CCCC6)c(F)c4)c23)C1] | N-[(4-{3-[(3R)-3- acetamidopyrrolidin-1-yl]- 1H-pyrazolo[3,4-b]pyridin- 4-yl}-2- fluorophenyl)methyl]- 4,5,6,7-tetrahydro-1,3- benzothiazole-2- carboxamide |
| 357 | 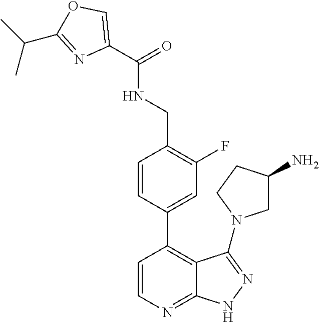 [SMILES: CC(C)c1nc(C(=O)NCc2ccc(-c3ccnc4[nH]nc(N5CC[C@@H](N)C5)c34)cc2F)co1] | N-[(4-{3-[(3R)-3- aminopyrrolidin-1-yl]-1H- pyrazolo[3,4-b]pyridin-4- yl}-2- fluorophenyl)methyl]-2- (propan-2-yl)-1,3-oxazole- 4-carboxamide |
| 359 | 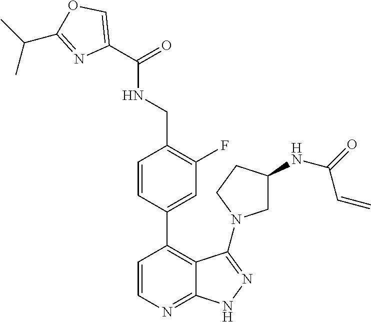 [SMILES: C=CC(=O)N[C@@H]1CCN(c2n[nH]c3nccc(-c4ccc(CNC(=O)c5coc(C(C)C)n5)c(F)c4)c23)C1] | N-{[2-fluoro-4-(3-{(3R)-3- [(prop-2- enoyl)amino]pyrrolidin-1- yl}-1H-pyrazolo[3,4- b]pyridin-4- yl)phenyl]methyl}-2- (propan-2-yl)-1,3-oxazole- 4-carboxamide |
| 360 | 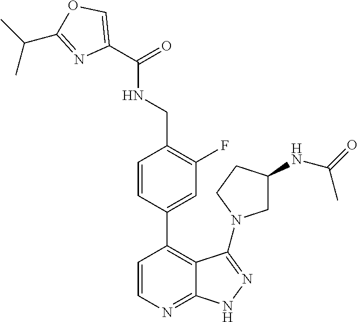 [SMILES: CC(=O)N[C@@H]1CCN(c2n[nH]c3nccc(-c4ccc(CNC(=O)c5coc(C(C)C)n5)c(F)c4)c23)C1] | N-[(4-{3-[(3R)-3- acetamidopyrrolidin-1-yl]- 1H-pyrazolo[3,4-b]pyridin- 4-yl}-2- fluorophenyl)methyl]-2- (propan-2-yl)-1,3-oxazole- 4-carboxamide |
| 362 | 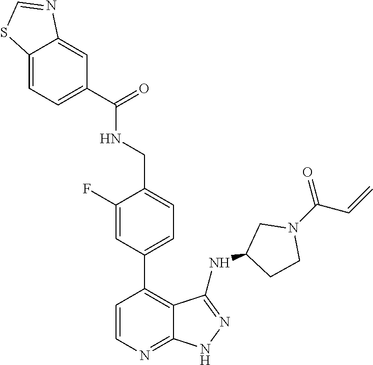 [SMILES: C=CC(=O)N1CC[C@@H](Nc2n[nH]c3nccc(-c4ccc(CNC(=O)c5ccc6scnc6c5)c(F)c4)c23)C1] | N-{[2-fluoro-4-(3-{[(3R)-1- (prop-2-enoyl)pyrrolidin-3- yl]amino}-1H- pyrazolo[3,4-b]pyridin-4- yl)phenyl]methyl}-1,3- benzothiazole-5- carboxamide |
| 363 | 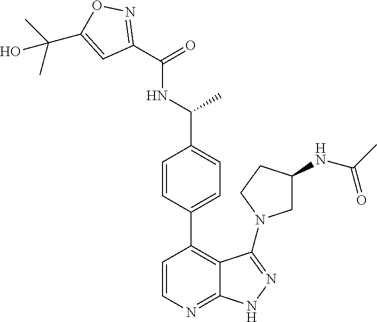 [SMILES: CC(=O)N[C@@H]1CCN(c2n[nH]c3nccc(-c4ccc([C@@H](C)NC(=O)c5cc(C(C)(C)O)on5)cc4)c23)C1] | N-[(1R)-1-(4-{3-[(3R)-3- acetamidopyrrolidin-1-yl]- 1H-pyrazolo[3,4-b]pyridin- 4-yl}phenyl)ethyl]-5-(2- hydroxypropan-2-yl)-1,2- oxazole-3-carboxamide |
| 364 | 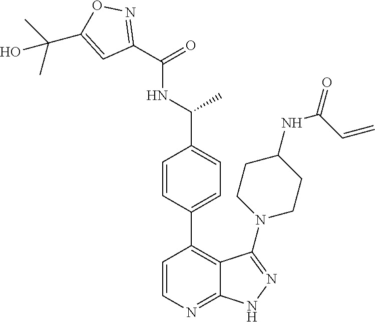 [SMILES: C=CC(=O)NC1CCN(c2n[nH]c3nccc(-c4ccc([C@@H](C)NC(=O)c5cc(C(C)(C)O)on5)cc4)c23)CC1] | 5-(2-hydroxypropan-2-yl)- N-{(1R)-1-[4-(3-{4-[(prop- 2-enoyl)amino]piperidin-1- yl}-1H-pyrazolo[3,4- b]pyridin-4- yl)phenyl]ethyl}-1,2- oxazole-3-carboxamide |
| 365 | 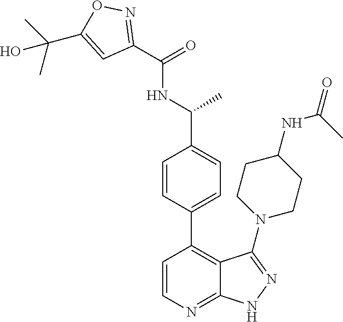 [SMILES: CC(=O)NC1CCN(c2n[nH]c3nccc(-c4ccc([C@@H](C)NC(=O)c5cc(C(C)(C)O)on5)cc4)c23)CC1] | N-[(1R)-1-{4-[3-(4- acetamidopiperidin-1-yl)- 1H-pyrazolo[3,4-b]pyridin- 4-yl]phenyl}ethyl]-5-(2- hydroxypropan-2-yl)-1,2- oxazole-3-carboxamide |
| 366 | 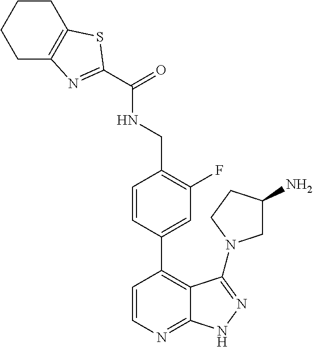 [SMILES: N[C@@H]1CCN(c2n[nH]c3nccc(-c4ccc(CNC(=O)c5nc6c(s5)CCCC6)c(F)c4)c23)C1] | N-[(4-{3-[(3R)-3- aminopyrrolidin-1-yl]-1H- pyrazolo[3,4-b]pyridin-4- yl}-2- fluorophenyl)methyl]- 4,5,6,7-tetrahydro-1,3- benzothiazole-2- carboxamide |
| 367 | 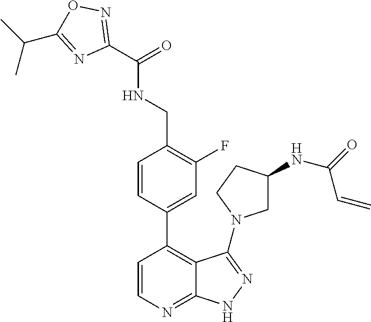 [SMILES: C=CC(=O)N[C@@H]1CCN(c2n[nH]c3nccc(-c4ccc(CNC(=O)c5noc(C(C)C)n5)c(F)c4)c23)C1] | N-{[2-fluoro-4-(3-{(3R)-3- [(prop-2- enoyl)amino]pyrrolidin-1- yl}-1H-pyrazolo[3,4- b]pyridin-4- yl)phenyl]methyl}-5- (propan-2-yl)-1,2,4- oxadiazole-3-carboxamide |
| 369 | 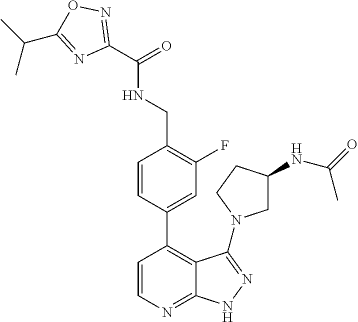 [SMILES: CC(=O)N[C@@H]1CCN(c2n[nH]c3nccc(-c4ccc(CNC(=O)c5noc(C(C)C)n5)c(F)c4)c23)C1] | N-[(4-{3-[(3R)-3- acetamidopyrrolidin-1-yl]- 1H-pyrazolo[3,4-b]pyridin- 4-yl}-2- fluorophenyl)methyl]-5- (propan-2-yl)-1,2,4- oxadiazole-3-carboxamide |
| 370 | 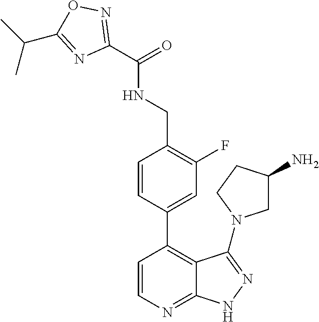 [SMILES: CC(C)c1nc(C(=O)NCc2ccc(-c3ccnc4[nH]nc(N5CC[C@@H](N)C5)c34)cc2F)no1] | N-[(4-{3-[(3R)-3- aminopyrrolidin-1-yl]-1H- pyrazolo[3,4-b]pyridin-4- yl}-2- fluorophenyl)methyl]-5- (propan-2-yl)-1,2,4- oxadiazole-3-carboxamide |
| 371 | 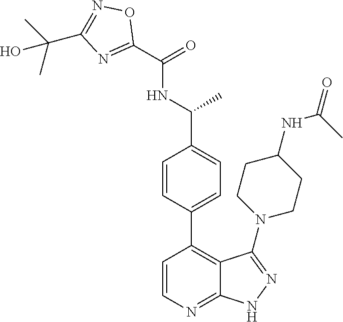 [SMILES: CC(=O)NC1CCN(c2n[nH]c3nccc(-c4ccc([C@@H](C)NC(=O)c5nc(C(C)(C)O)no5)cc4)c23)CC1] | N-[(1R)-1-{4-[3-(4- acetamidopiperidin-1-yl)- 1H-pyrazolo[3,4-b]pyridin- 4-yl]phenyl}ethyl]-3-tert- butyl-1,2,4-oxadiazole-5- carboxamide |
| 372 | 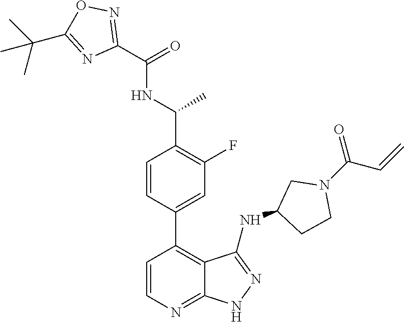 [SMILES: C=CC(=O)N1CC[C@@H](Nc2n[nH]c3nccc(-c4ccc([C@@H](C)NC(=O)c5noc(C(C)(C)C)n5)c(F)c4)c23)C1] | 5-tert-butyl-N-{(1R)-1-[2- fluoro-4-(3-{[(3R)-1-(prop- 2-enoyl)pyrrolidin-3- yl]amino}-1H- pyrazolo[3,4-b]pyridin-4- yl)phenyl]ethyl}-1,2,4- oxadiazole-3-carboxamide |
| 373 | 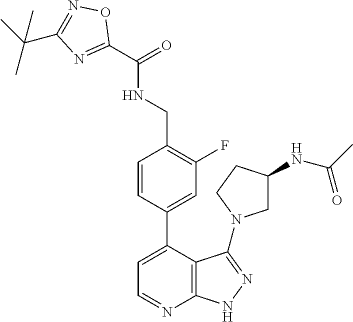 [SMILES: CC(=O)N[C@@H]1CCN(c2n[nH]c3nccc(-c4ccc(CNC(=O)c5nc(C(C)(C)C)no5)c(F)c4)c23)C1] | N-[(4-{3-[(3R)-3- acetamidopyrrolidin-1-yl]- 1H-pyrazolo[3,4-b]pyridin- 4-yl}-2- fluorophenyl)methyl]-3- tert-butyl-1,2,4-oxadiazole- 5-carboxamide |
| 374 | 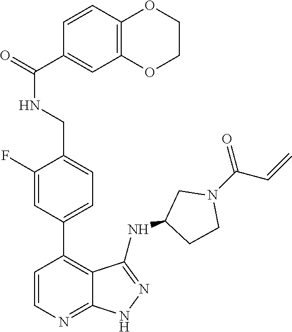 [SMILES: C=CC(=O)N1CC[C@@H](Nc2n[nH]c3nccc(-c4ccc(CNC(=O)c5ccc6c(c5)OCCO6)c(F)c4)c23)C1] | N-{[2-fluoro-4-(3-{[(3R)-1- (prop-2-enoyl)pyrrolidin-3- yl]amino}-1H- pyrazolo[3,4-b]pyridin-4- yl)phenyl]methyl}-2,3- dihydro-1,4-benzodioxine- 6-carboxamide |
| 375 | 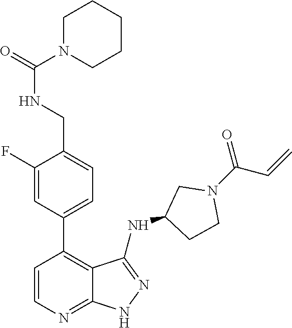 [SMILES: C=CC(=O)N1CC[C@@H](Nc2n[nH]c3nccc(-c4ccc(CNC(=O)N5CCCCC5)c(F)c4)c23)C1] | N-{[2-fluoro-4-(3-{[(3R)-1- (prop-2-enoyl)pyrrolidin-3- yl]amino}-1H- pyrazolo[3,4-b]pyridin-4- yl)phenyl]methyl}piperidine- 1-carboxamide |
| 376 | 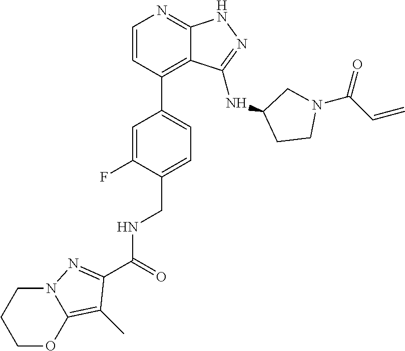 [SMILES: C=CC(=O)N1CC[C@@H](Nc2n[nH]c3nccc(-c4ccc(CNC(=O)c5nn6c(c5C)OCCC6)c(F)c4)c23)C1] | N-{[2-fluoro-4-(3-{[(3R)-1- (prop-2-enoyl)pyrrolidin-3- yl]amino}-1H- pyrazolo[3,4-b]pyridin-4- yl)phenyl]methyl}-3- methyl-6,7-dihydro-5H- pyrazolo[5,1- b][1,3]oxazine-2- carboxamide |
| 377 | 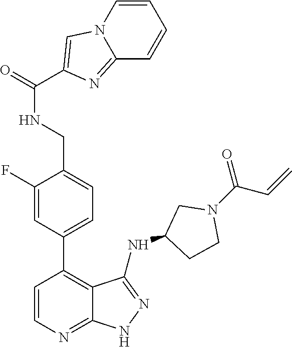 [SMILES: C=CC(=O)N1CC[C@@H](Nc2n[nH]c3nccc(-c4ccc(CNC(=O)c5cn6ccccc6n5)c(F)c4)c23)C1] | N-{[2-fluoro-4-(3-{[(3R)-1- (prop-2-enoyl)pyrrolidin-3- yl]amino}-1H- pyrazolo[3,4-b]pyridin-4- yl)phenyl]methyl}imidazo [1,2-a]pyridine-2- carboxamide |
| 378 | 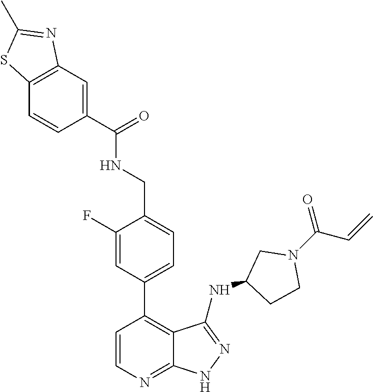 [SMILES: C=CC(=O)N1CC[C@@H](Nc2n[nH]c3nccc(-c4ccc(CNC(=O)c5ccc6sc(C)nc6c5)c(F)c4)c23)C1] | N-{[2-fluoro-4-(3-{[(3R)-1- (prop-2-enoyl)pyrrolidin-3- yl]amino}-1H- pyrazolo[3,4-b]pyridin-4- yl)phenyl]methyl}-2- methyl-1,3-benzothiazole- 5-carboxamide |
| 379 | 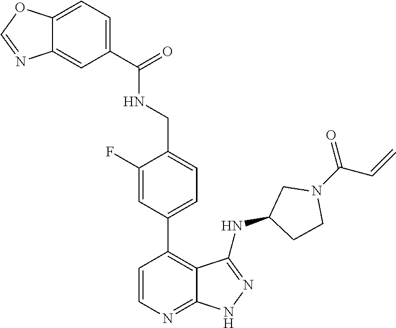 [SMILES: C=CC(=O)N1CC[C@@H](Nc2n[nH]c3nccc(-c4ccc(CNC(=O)c5ccc6ocnc6c5)c(F)c4)c23)C1] | (R)-N-{[2-fluoro-4-(3-{[1- (prop-2-enoyl)pyrrolidin-3- yl]amino}-1H- pyrazolo[3,4-b]pyridin-4- yl)phenyl]methyl}-1,3- benzoxazole-5- carboxamide |
| 380 | 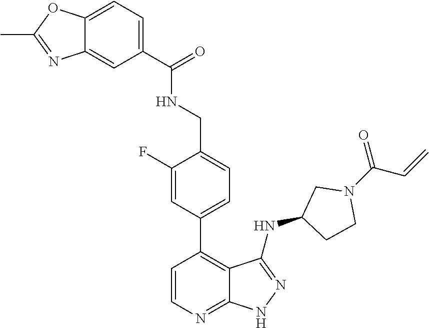 [SMILES: C=CC(=O)N1CC[C@@H](Nc2n[nH]c3nccc(-c4ccc(CNC(=O)c5ccc6oc(C)nc6c5)c(F)c4)c23)C1] | (R)-N-{[2-fluoro-4-(3-{[1- (prop-2-enoyl)pyrrolidin-3- yl]amino}-1H- pyrazolo[3,4-b]pyridin-4- yl)phenyl]methyl}-2- methyl-1,3-benzoxazole-5- carboxamide |
| 381 | 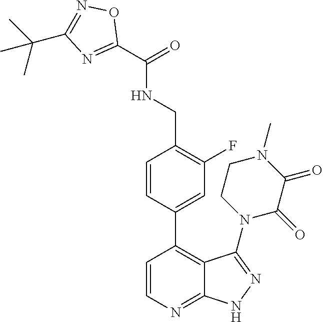 [SMILES: CN1CCN(c2n[nH]c3nccc(-c4ccc(CNC(=O)c5nc(C(C)(C)C)no5)c(F)c4)c23)C(=O)C1=O] | 3-tert-butyl-N-({2-fluoro-4- [3-(4-methyl-2,3- dioxopiperazin-1-yl)-1H- pyrazolo[3,4-b]pyridin-4- yl]phenyl}methyl)-1,2,4- oxadiazole-5-carboxamide |
| 382 | 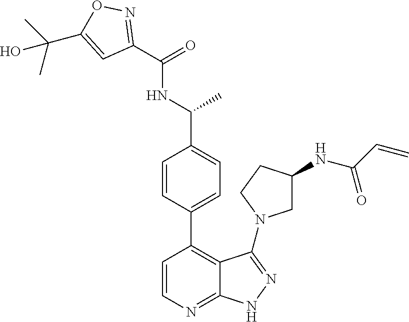 [SMILES: C=CC(=O)N[C@@H]1CCN(c2n[nH]c3nccc(-c4ccc([C@@H](C)NC(=O)c5cc(C(C)(C)O)on5)cc4)c23)C1] | 5-(2-hydroxypropan-2-yl)- N-{(1R)-1-[4-(3-{(3R)-3- [(prop-2- enoyl)amino]pyrrolidin-1- yl}-1H-pyrazolo[3,4- b]pyridin-4- yl)phenyl]ethyl}-1,2- oxazole-3-carboxamide |
| 384 | 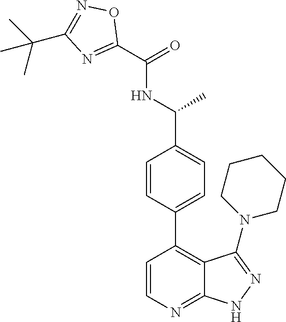 [SMILES: C[C@@H](NC(=O)c1nc(C(C)(C)C)no1)c1ccc(-c2ccnc3[nH]nc(N4CCCCC4)c23)cc1] | 3-tert-butyl-N-[(1R)-1-{4- [3-(piperidin-1-yl)-1H- pyrazolo[3,4-b]pyridin-4- yl]phenyl}ethyl]-1,2,4- oxadiazole-5-carboxamide |
| 385 | 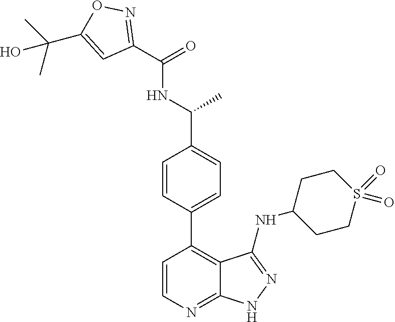 [SMILES: C[C@@H](NC(=O)c1cc(C(C)(C)O)on1)c1ccc(-c2ccnc3[nH]nc(NC4CCS(=O)(=O)CC4)c23)cc1] | N-[(1R)-1-(4-{3-[(1,1- dioxo-1λ⁶-thian-4- yl)amino]-1H- pyrazolo[3,4-b]pyridin-4- yl}phenyl)ethyl]-5-(2- hydroxypropan-2-yl)-1,2- oxazole-3-carboxamide |
| 386 | 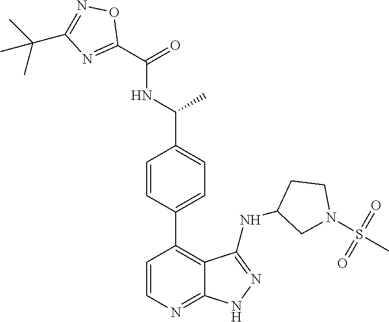 [SMILES: C[C@@H](NC(=O)c1nc(C(C)(C)C)no1)c1ccc(-c2ccnc3[nH]nc(NC4CCN(S(C)(=O)=O)C4)c23)cc1] | 3-tert-butyl-N-{(1R)-1-[4- (3-{[1- (methanesulfonyl)pyrrolidin- 3-yl]amino}-1H- pyrazolo[3,4-b]pyridin-4- yl)phenyl]ethyl}-1,2,4- oxadiazole-5-carboxamide |
| 387 | 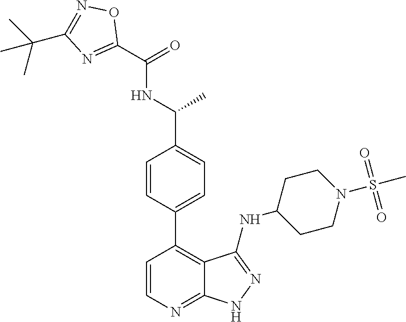 [SMILES: C[C@@H](NC(=O)c1nc(C(C)(C)C)no1)c1ccc(-c2ccnc3[nH]nc(NC4CCN(S(C)(=O)=O)CC4)c23)cc1] | 3-tert-butyl-N-{(1R)-1-[4- (3-{[1- (methanesulfonyl)piperidin- 4-yl]amino}-1H- pyrazolo[3,4-b]pyridin-4- yl)phenyl]ethyl}-1,2,4- oxadiazole-5-carboxamide |
| 389 | 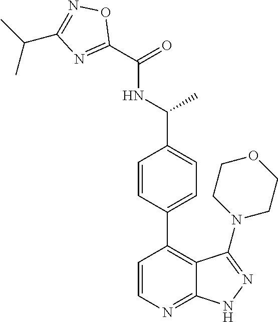 [SMILES: CC(C)c1noc(C(=O)N[C@H](C)c2ccc(-c3ccnc4[nH]nc(N5CCOCC5)c34)cc2)n1] | N-[(1R)-1-{4-[3- (morpholin-4-yl)-1H- pyrazolo[3,4-b]pyridin-4- yl]phenyl}ethyl]-3- (propan-2-yl)-1,2,4- oxadiazole-5-carboxamide |
| 390 | 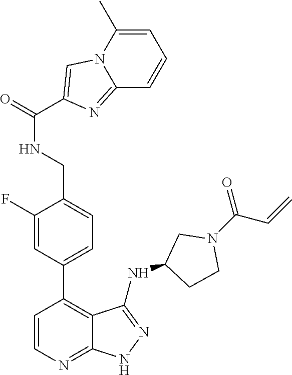 [SMILES: C=CC(=O)N1CC[C@@H](Nc2n[nH]c3nccc(-c4ccc(CNC(=O)c5cn6c(C)cccc6n5)c(F)c4)c23)C1] | N-{[2-fluoro-4-(3-{[(3R)-1- (prop-2-enoyl)pyrrolidin-3- yl]amino}-1H- pyrazolo[3,4-b]pyridin-4- yl)phenyl]methyl}-5- methylimidazo[1,2- a]pyridine-2-carboxamide |
| 391 | 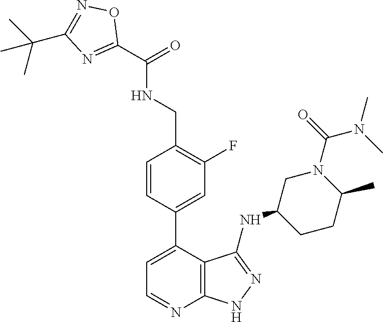 [SMILES: C[C@H]1CC[C@@H](Nc2n[nH]c3nccc(-c4ccc(CNC(=O)c5nc(C(C)(C)C)no5)c(F)c4)c23)CN1C(=O)N(C)C] | (2S,5R)-5-{[4-(4-{[(3-tert- butyl-1,2,4-oxadiazole-5- carbonyl)amino]methyl}-3- fluorophenyl)-1H- pyrazolo[3,4-b]pyridin-3- yl]amino}-N,N,2- trimethylpiperidine-1- carboxamide |
| 392 | 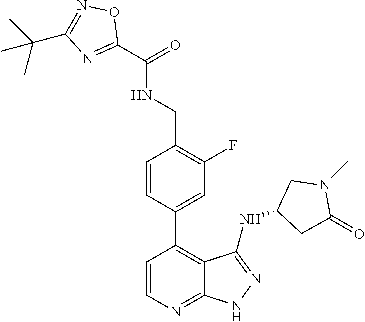 [SMILES: CN1C[C@@H](Nc2n[nH]c3nccc(-c4ccc(CNC(=O)c5nc(C(C)(C)C)no5)c(F)c4)c23)CC1=O] | 3-tert-butyl-N-{[2-fluoro-4- (3-{[(3S)-1-methyl-5- oxopyrrolidin-3-yl]amino}- 1H-pyrazolo[3,4-b]pyridin- 4-yl)phenyl]methyl}-1,2,4- oxadiazole-5-carboxamide |
| 393 | 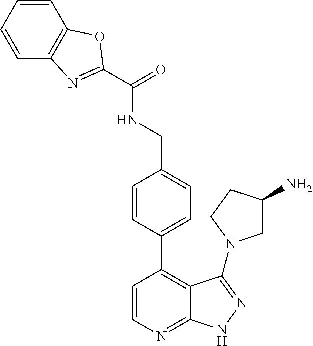 [SMILES: N[C@@H]1CCN(c2n[nH]c3nccc(-c4ccc(CNC(=O)c5nc6ccccc6o5)cc4)c23)C1] | N-[(4-{3-[(3R)-3- aminopyrrolidin-1-yl]-1H- pyrazolo[3,4-b]pyridin-4- yl}-2- fluorophenyl)methyl]-1,3- benzoxazole-2- carboxamide |
| 394 | 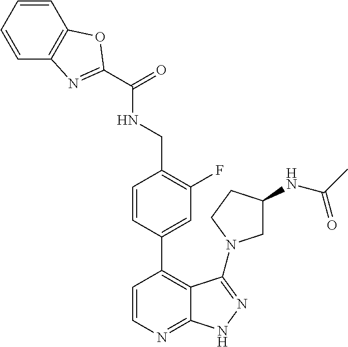 [SMILES: CC(=O)N[C@@H]1CCN(c2n[nH]c3nccc(-c4ccc(CNC(=O)c5nc6ccccc6o5)c(F)c4)c23)C1] | N-[(4-{3-[(3R)-3- acetamidopyrrolidin-1-yl]- 1H-pyrazolo[3,4-b]pyridin- 4-yl}-2- fluorophenyl)methyl]-1,3- benzoxazole-2- carboxamide |
| 395 | 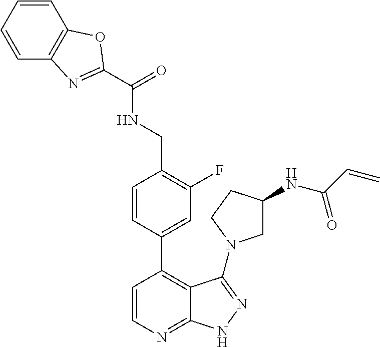 [SMILES: C=CC(=O)N[C@@H]1CCN(c2n[nH]c3nccc(-c4ccc(CNC(=O)c5nc6ccccc6o5)c(F)c4)c23)C1] | N-{[2-fluoro-4-(3-{(3R)-3- [(prop-2- enoyl)amino]pyrrolidin-1- yl}-1H-pyrazolo[3,4- b]pyridin-4- yl)phenyl]methyl}-1,3- benzoxazole-2- carboxamide |
| 396 | 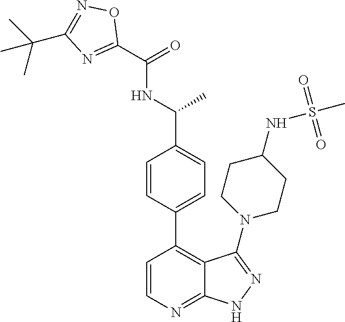 [SMILES: C[C@@H](NC(=O)c1nc(C(C)(C)C)no1)c1ccc(-c2ccnc3[nH]nc(N4CCC(NS(C)(=O)=O)CC4)c23)cc1] | 3-tert-butyl-N-{(1R)-1-[4- (3-{4- [(methanesulfonyl)amino] piperidin-1-yl}-1H- pyrazolo[3,4-b]pyridin-4- yl)phenyl]ethyl}-1,2,4- oxadiazole-5-carboxamide |
| 397 | 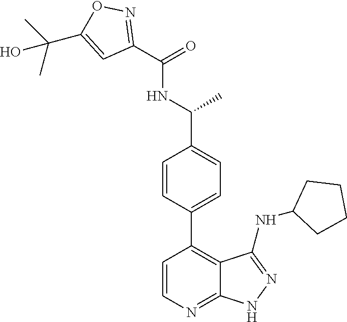 [SMILES: C[C@@H](NC(=O)c1cc(C(C)(C)O)on1)c1ccc(-c2ccnc3[nH]nc(NC4CCCC4)c23)cc1] | N-[(1R)-1-{4-[3- (cyclopentylamino)-1H- pyrazolo[3,4-b]pyridin-4- yl]phenyl}ethyl]-5-(2- hydroxypropan-2-yl)-1,2- oxazole-3-carboxamide |
| 398 | 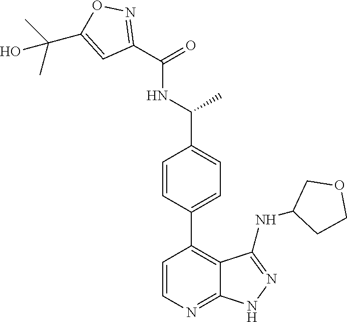 [SMILES: C[C@@H](NC(=O)c1cc(C(C)(C)O)on1)c1ccc(-c2ccnc3[nH]nc(NC4CCOC4)c23)cc1] | 5-(2-hydroxypropan-2-yl)- N-[(1R)-1-(4-{3-[(oxolan- 3-yl)amino]-1H- pyrazolo[3,4-b]pyridin-4- yl}phenyl)ethyl]-1,2- oxazole-3-carboxamide |
| 399 | 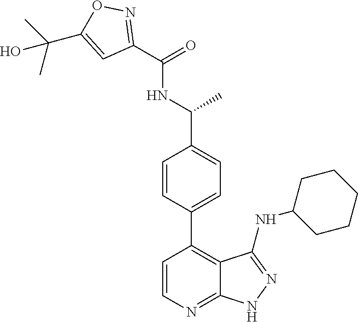 [SMILES: C[C@@H](NC(=O)c1cc(C(C)(C)O)on1)c1ccc(-c2ccnc3[nH]nc(NC4CCCCC4)c23)cc1] | N-[(1R)-1-{4-[3- (cyclohexylamino)-1H- pyrazolo[3,4-b]pyridin-4- yl]phenyl}ethyl]-5-(2- hydroxypropan-2-yl)-1,2- oxazole-3-carboxamide |
| 400 | 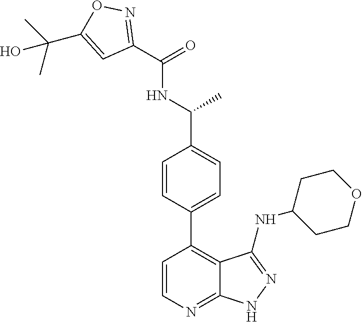 [SMILES: C[C@@H](NC(=O)c1cc(C(C)(C)O)on1)c1ccc(-c2ccnc3[nH]nc(NC4CCOCC4)c23)cc1] | 5-(2-hydroxypropan-2-yl)- N-[(1R)-1-(4-{3-[(oxan-4- yl)amino]-1H- pyrazolo[3,4-b]pyridin-4- yl}phenyl)ethyl]-1,2- oxazole-3-carboxamide |
| 401 | 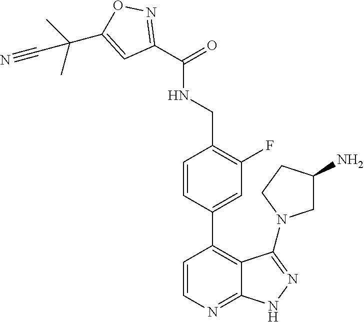 [SMILES: CC(C)(C#N)c1cc(C(=O)NCc2ccc(-c3ccnc4[nH]nc(N5CC[C@@H](N)C5)c34)cc2F)no1] | N-[(4-{3-[(3R)-3- aminopyrrolidin-1-yl]-1H- pyrazolo[3,4-b]pyridin-4- yl}-2- fluorophenyl)methyl]-5-(2- cyanopropan-2-yl)-1,2- oxazole-3-carboxamide |
| 402 | 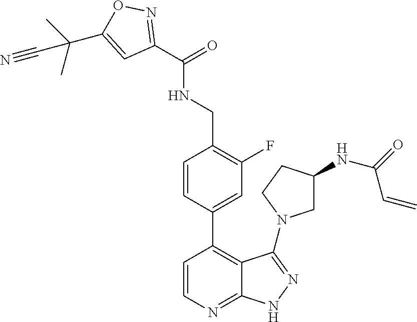 [SMILES: C=CC(=O)N[C@@H]1CCN(c2n[nH]c3nccc(-c4ccc(CNC(=O)c5cc(C(C)(C)C#N)on5)c(F)c4)c23)C1] | 5-(2-cyanopropan-2-yl)-N- {[2-fluoro-4-(3-{(3R)-3- [(prop-2- noyl)amino]pyrrolidin-1- yl}-1H-pyrazolo[3,4- b]pyridin-4- yl)phenyl]methyl}-1,2- oxazole-3-carboxamide |
| 403 | 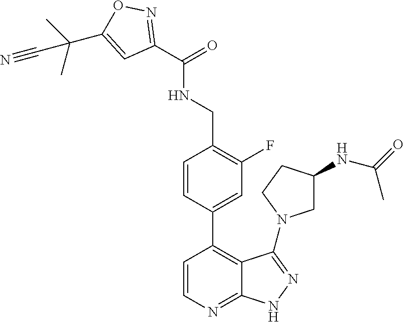 [SMILES: CC(=O)N[C@@H]1CCN(c2n[nH]c3nccc(-c4ccc(CNC(=O)c5cc(C(C)(C)C#N)on5)c(F)c4)c23)C1] | N-[(4-{3-[(3R)-3- acetamidopyrrolidin-1-yl]- 1H-pyrazolo[3,4-b]pyridin- 4-yl}-2- fluorophenyl)methyl]-5-(2- cyanopropan-2-yl)-1,2- oxazole-3-carboxamide |
| 404 | 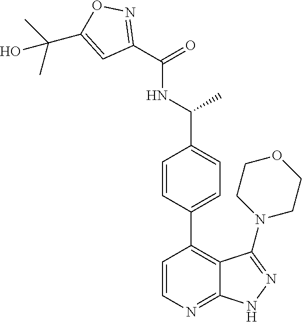 [SMILES: C[C@@H](NC(=O)c1cc(C(C)(C)O)on1)c1ccc(-c2ccnc3[nH]nc(N4CCOCC4)c23)cc1] | 5-(2-hydroxypropan-2-yl)- N-[(1R)-1-{4-[3- (morpholin-4-yl)-1H- pyrazolo[3,4-b]pyridin-4- yl]phenyl}ethyl]-1,2- oxazole-3-carboxamide |
| 405 | 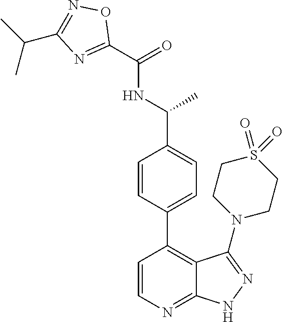 [SMILES: CC(C)c1noc(C(=O)N[C@H](C)c2ccc(-c3ccnc4[nH]nc(N5CCS(=O)(=O)CC5)c34)cc2)n1] | N-[(1R)-1-{4-[3-(1,1- dioxo-1λ⁶-thiomorpholin-4- yl)-1H-pyrazolo[3,4- b]pyridin-4- yl]phenyl}ethyl]-3- (propan-2-yl)-1,2,4- oxadiazole-5-carboxamide |
| 406 | 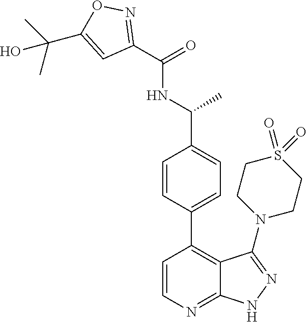 [SMILES: C[C@@H](NC(=O)c1cc(C(C)(C)O)on1)c1ccc(-c2ccnc3[nH]nc(N4CCS(=O)(=O)CC4)c23)cc1] | N-[(1R)-1-{4-[3-(1,1- dioxo-1λ⁶-thiomorpholin-4- yl)-1H-pyrazolo[3,4- b]pyridin-4- yl]phenyl}ethyl]-5-(2- hydroxypropan-2-yl)-1,2- oxazole-3-carboxamide |
| 408 | 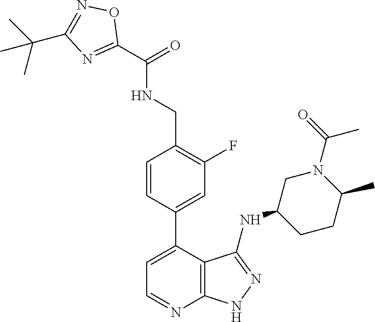 [SMILES: CC(=O)N1C[C@H](Nc2n[nH]c3nccc(-c4ccc(CNC(=O)c5nc(C(C)(C)C)no5)c(F)c4)c23)CC[C@@H]1C] | N-{[4-(3-{[(3R,6S)-1- acetyl-6-methylpiperidin-3- yl]amino}-1H- pyrazolo[3,4-b]pyridin-4- yl)-2- fluorophenyl]methyl}-3- tert-butyl-1,2,4-oxadiazole- 5-carboxamide |
| 409 | 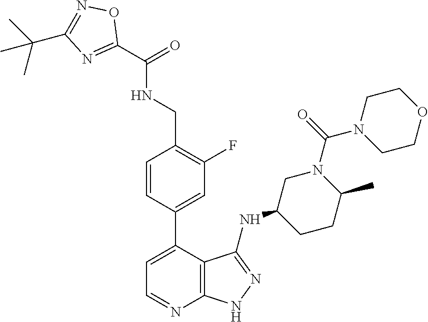 [SMILES: C[C@H]1CC[C@@H](Nc2n[nH]c3nccc(-c4ccc(CNC(=O)c5nc(C(C)(C)C)no5)c(F)c4)c23)CN1C(=O)N1CCOCC1] | 3-tert-butyl-N-{[2-fluoro-4- (3-{[(3R,6S)-6-methyl-1- (morpholine-4- carbonyl)piperidin-3- yl]amino}-1H- pyrazolo[3,4-b]pyridin-4- yl)phenyl]methyl}-1,2,4- oxadiazole-5-carboxamide |
| 410 | 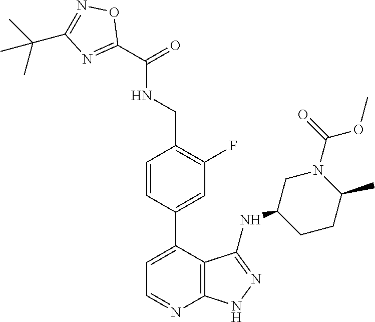 [SMILES: COC(=O)N1C[C@H](Nc2n[nH]c3nccc(-c4ccc(CNC(=O)c5nc(C(C)(C)C)no5)c(F)c4)c23)CC[C@@H]1C] | methyl (2S,5R)-5-{[4-(4- {[(3-tert-butyl-1,2,4- oxadiazole-5- carbonyl)amino]methyl}-3- fluorophenyl)-1H- pyrazolo[3,4-b]pyridin-3- yl]amino}-2- methylpiperidine-1- carboxylate |
| 411 | 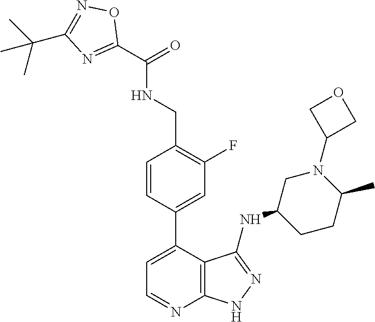 [SMILES: C[C@H]1CC[C@@H](Nc2n[nH]c3nccc(-c4ccc(CNC(=O)c5nc(C(C)(C)C)no5)c(F)c4)c23)CN1C1COC1] | 3-tert-butyl-N-{[2-fluoro-4- (3-{[(3R,6S)-6-methyl-1- (oxetan-3-yl)piperidin-3- yl]amino}-1H- pyrazolo[3,4-b]pyridin-4- yl)phenyl]methyl}-1,2,4- oxadiazole-5-carboxamide |
| 414 | 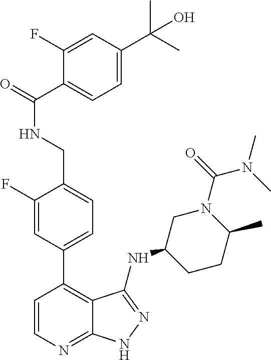 [SMILES: C[C@H]1CC[C@@H](Nc2n[nH]c3nccc(-c4ccc(CNC(=O)c5ccc(C(C)(C)O)cc5F)c(F)c4)c23)CN1C(=O)N(C)C] | (2S,5R)-5-{[4-(3-fluoro-4- {[2-fluoro-4-(2- hydroxypropan-2- yl)benzamido]methyl} phenyl)-1H-pyrazolo[3,4- b]pyridin-3-yl]amino}- N,N,2-trimethylpiperidine- 1-carboxamide |
| 415 | 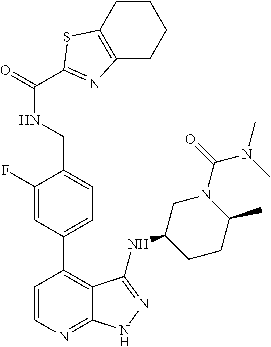 [SMILES: C[C@H]1CC[C@@H](Nc2n[nH]c3nccc(-c4ccc(CNC(=O)c5nc6c(s5)CCCC6)c(F)c4)c23)CN1C(=O)N(C)C] | N-{[4-(3-{[(3R,6S)-1- (dimethylcarbamoyl)-6- methylpiperidin-3- yl]amino}-1H- pyrazolo[3,4-b]pyridin-4- yl)-2- fluorophenyl]methyl}- 4,5,6,7-tetrahydro-1,3- benzothiazole-2- carboxamide |
| 418 | 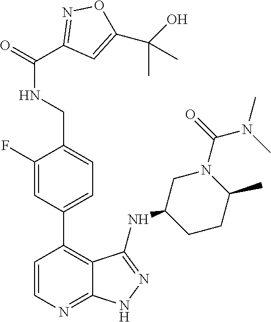 [SMILES: C[C@H]1CC[C@@H](Nc2n[nH]c3nccc(-c4ccc(CNC(=O)c5cc(C(C)(C)O)on5)c(F)c4)c23)CN1C(=O)N(C)C] | (2S,5R)-5-({4-[3-fluoro-4- ({[5-(2-hydroxypropan-2- yl)-1,2-oxazole-3- carbonyl]amino}methyl) phenyl]-1H-pyrazolo[3,4- b]pyridin-3-yl}amino)- N,N,2-trimethylpiperidine- 1-carboxamide |
| 419 | 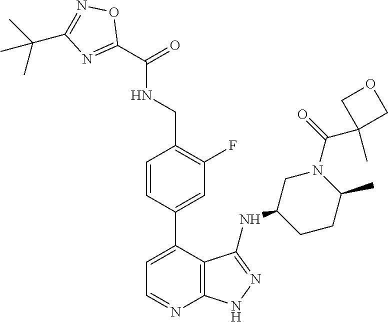 [SMILES: C[C@H]1CC[C@@H](Nc2n[nH]c3nccc(-c4ccc(CNC(=O)c5nc(C(C)(C)C)no5)c(F)c4)c23)CN1C(=O)C1(C)COC1] | 3-tert-butyl-N-{[2-fluoro-4- (3-{[(3R,6S)-6-methyl-1- (3-methyloxetane-3- carbonyl)piperidin-3- yl]amino}-1H- pyrazolo[3,4-b]pyridin-4- yl)phenyl]methyl}-1,2,4- oxadiazole-5-carboxamide |
| 420 | 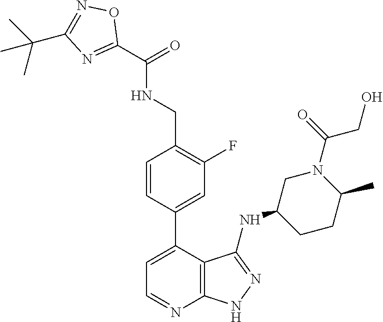 [SMILES: C[C@H]1CC[C@@H](Nc2n[nH]c3nccc(-c4ccc(CNC(=O)c5nc(C(C)(C)C)no5)c(F)c4)c23)CN1C(=O)CO] | 3-tert-butyl-N-{[2-fluoro-4- (3-{[(3R,6S)-1- (hydroxyacetyl)-6- methylpiperidin-3- yl]amino}-1H- pyrazolo[3,4-b]pyridin-4- yl)phenyl]methyl}-1,2,4- oxadiazole-5-carboxamide |
| 421 | 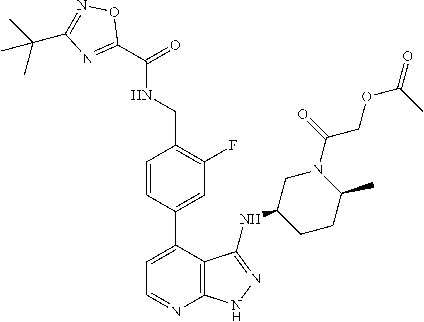 [SMILES: CC(=O)OCC(=O)N1C[C@H](Nc2n[nH]c3nccc(-c4ccc(CNC(=O)c5nc(C(C)(C)C)no5)c(F)c4)c23)CC[C@@H]1C] | 2-[(2S,5R)-5-{[4-(4-{[(3- tert-butyl-1,2,4-oxadiazole- 5-carbonyl)amino]methyl}- 3-fluorophenyl)-1H- pyrazolo[3,4-b]pyridin-3- yl]amino}-2- methylpiperidin-1-yl]-2- oxoethyl acetate |
| 423 | 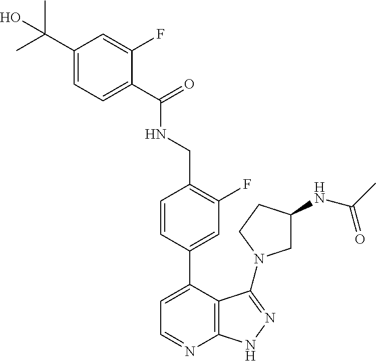 [SMILES: CC(=O)N[C@@H]1CCN(c2n[nH]c3nccc(-c4ccc(CNC(=O)c5ccc(C(C)(C)O)cc5F)c(F)c4)c23)C1] | N-[(4-{3-[(3R)-3- acetamidopyrrolidin-1-yl]- 1H-pyrazolo[3,4-b]pyridin- 4-yl}-2- fluorophenyl)methyl]-2- fluoro-4-(2-hydroxypropan- 2-yl)benzamide |
| 424 | 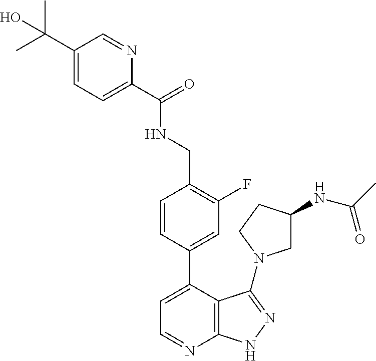 [SMILES: CC(=O)N[C@@H]1CCN(c2n[nH]c3nccc(-c4ccc(CNC(=O)c5ccc(C(C)(C)O)cn5)c(F)c4)c23)C1] | N-[(4-{3-[(3R)-3- acetamidopyrrolidin-1-yl]- 1H-pyrazolo[3,4-b]pyridin- 4-yl}-2- fluorophenyl)methyl]-5-(2- hydroxypropan-2- yl)pyridine-2-carboxamide |

TABLE 2
Additional NMR Data for representative compounds of the invention

| ID | NMR |
|---|---|
| 52 | ¹H NMR (400 MHz, DMSO) δ 12.58 (s, 1H), 8.44 (d, J = 4.7 Hz, 1H), 8.33 (s, 1H), |
| | 7.65-7.44 (m, 4H), 7.00 (d, J = 4.8 Hz, 1H), 6.45 (s, 1H), 4.77 (s, 2H), 4.63 (s, 1H), |
| | 4.13 (s, 1H), 4.06-4.00 (m, 2H), 3.67-3.64 (m, 2H), 3.22-2.90 (m, 8H), 2.13-2.03 (m, 1H), |
| | 1.73-1.63 (m, J = 5.3 Hz, 1H), 1.19 (s, 6H). |
| 69 | ¹H NMR (400 MHz, DMSO) δ 12.49 (s, 1H), 9.56 (t, J = 6.0 Hz, 1H), 8.43 (d, J = 4.7 Hz, |
| | 1H), 8.31 (s, 1H), 7.61-7.43 (m, 3H), 6.97 (d, J = 4.7 Hz, 1H), 4.59 (d, J = 5.9 Hz, |
| | 2H), 4.29 (d, J = 7.4 Hz, 1H), 3.64 (s, 1H), 3.10 (d, J = 9.2 Hz, 1H), 2.80 (s, 1H), |
| | 2.67-2.58 (m, 2H), 1.81 (s, 1H), 1.57 (s, J = 14.2 Hz, 1H), 1.43 (s, 11H). |
| 70 | ¹H NMR (400 MHz, DMSO) δ 12.40 (s, 1H), 9.79 (s, 1H), 8.40 (d, J = 4.7 Hz, 1H), |
| | 8.31 (s, 1H), 7.57 (d, J = 8.2 Hz, 2H), 7.40 (d, J = 8.3 Hz, 2H), 6.92 (d, J = 4.7 Hz, 1H), |
| | 4.13 (d, J = 7.5 Hz, 1H), 3.62 (s, 1H), 3.09 (d, J = 11.6 Hz, 1H), 2.78 (s, 1H), 2.59 (s, 2H), |
| | 1.79 (s, 1H), 1.60-1.17 (m, 16H). |
| 71 | ¹H NMR (400 MHz, DMSO) δ 12.54 (s, 1H), 10.20 (s, 1H), 8.48-8.27 (m, 2H), 7.60 (d, |
| | J = 8.1 Hz, 2H), 7.40 (d, J = 8.1 Hz, 2H), 6.95 (d, J = 4.7 Hz, 1H), 4.44 (d, J = 5.5 Hz, |
| | 1H), 4.14 (s, 1H), 3.29-3.20 (m, 1H), 3.14-2.94 (m, 3H), 2.16-2.05 (m, 1H), |
| | 1.71-1.61 (m, 1H), 1.49-1.26 (m, 13H). |
| 72 | ¹H NMR (400 MHz, DMSO) δ 12.54 (s, 1H), 9.52 (d, J = 8.1 Hz, 1H), 8.42 (d, J = 4.7 Hz, |
| | 1H), 8.36 (s, 1H), 7.64 (d, J = 8.2 Hz, 2H), 7.58 (d, J = 8.2 Hz, 2H), 6.97 (d, J = 4.7 Hz, |
| | 1H), 5.24 (p, J = 7.0 Hz, 1H), 4.38 (d, J = 5.8 Hz, 1H), 4.11 (s, 1H), 3.21 (s, 1H), |
| | 3.14-2.87 (m, 3H), 2.14-2.03 (m, 1H), 1.70-1.50 (m, 4H), 1.43 (s, 9H). |
| 73 | ¹H NMR (400 MHz, DMSO) δ 12.50 (s, 1H), 9.49 (d, J = 8.1 Hz, 1H), 8.42 (d, J = 4.7 Hz, |
| | 1H), 8.33 (s, 1H), 7.63 (d, J = 8.3 Hz, 2H), 7.58 (d, J = 8.2 Hz, 2H), 6.96 (d, J = 4.7 Hz, |
| | 1H), 5.27-5.23 (m, 1H), 4.28 (d, J = 5.9 Hz, 1H), 4.07 (s, 1H), 3.16-3.13 (m, 1H), |
| | 3.00-2.86 (m, 3H), 2.09-2.03 (m, 1H), 1.56 (d, J = 7.0 Hz, 4H), 1.43 (s, 9H). |
| 74 | ¹H NMR (400 MHz, DMSO) δ 12.60 (s, 1H), 9.73 (s, 1H), 8.44 (d, J = 4.7 Hz, 1H), |
| | 8.34 (s, 1H), 7.48-7.40 (m, 2H), 7.30 (d, J = 9.4 Hz, 1H), 6.99 (d, J = 4.7 Hz, 1H), |
| | 4.63-4.51 (m, 3H), 4.17 (d, J = 5.8 Hz, 1H), 3.24-3.20 (m, 1H), 3.14-3.08 (m, 1H), |
| | 3.05-2.97 (m, 2H), 2.10 (dq, J = 14.6, 7.3 Hz, 1H), 1.73 (td, J = 12.7, 7.0 Hz, 1H), 1.41 (s, |
| | 9H). |
| 75 | ¹H NMR (400 MHz, DMSO) δ 12.61 (s, 1H), 9.17 (t, J = 5.8 Hz, 1H), 8.44 (d, J = 4.7 Hz, |
| | 1H), 7.85 (d, J = 8.5 Hz, 2H), 7.49 (d, J = 8.5 Hz, 2H), 7.45 (s, 1H), 7.41 (d, J = 9.2 Hz, |
| | 1H), 7.28 (d, J = 9.7 Hz, 1H), 7.00 (d, J = 4.7 Hz, 1H), 4.65-4.53 (m, 3H), 4.18 (d, |
| | J = 5.4 Hz, 1H), 3.27-3.23 (m, 1H), 3.17-3.11 (m, 1H), 3.09-3.01 (m, 2H), 2.11 (dt, |
| | J = 20.5, 7.4 Hz, 1H), 1.76 (dt, J = 10.6, 5.5 Hz, 1H), 1.29 (s, 9H). |
| 91 | ¹H NMR (400 MHz, DMSO) δ 12.48 (s, 1H), 8.84 (d, J = 7.9 Hz, 1H), 8.41 (d, J = 4.7 Hz, |
| | 1H), 7.85 (d, J = 8.5 Hz, 2H), 7.61 (d, J = 8.3 Hz, 2H), 7.56 (d, J = 8.2 Hz, 2H), |
| | 7.49 (d, J = 8.5 Hz, 2H), 6.95 (d, J = 4.8 Hz, 1H), 5.29-5.20 (m, 1H), 4.27 (d, J = 6.0 Hz, |
| | 1H), 4.07 (d, J = 5.9 Hz, 1H), 3.16-3.12 (m, 1H), 2.96-2.84 (m, 3H), 2.06-1.99 (m, |
| | 1H), 1.54 (d, J = 7.1 Hz, 3H), 1.30 (s, 9H). |
| 100 | ¹H NMR (400 MHz, MeOD) δ 8.43 (d, J = 4.8 Hz, 1H), 7.87-7.77 (m, 2H), |
| | 7.58-7.49 (m, 3H), 7.49-7.42 (m, 2H), 7.08 (d, J = 4.8, 2.9 Hz, 1H), 4.71 (s, 2H), 3.09 (dd, J = 9.9, |
| | 6.3 Hz, 1H), 2.99-2.91 (m, 1H), 2.88-2.80 (m, 1H), 2.64 (dd, J = 9.9, 5.0 Hz, |
| | 1H), 2.04-1.95 (m, 1H), 1.46-1.38 (m, 1H), 1.35 (s, 9H), 0.92-0.84 (m, 1H). |
| 101 | ¹H NMR (400 MHz, DMSO) δ 12.84 (s, 1H), 9.01 (t, J = 5.8 Hz, 1H), 8.46 (d, J = 4.7 Hz, |
| | 1H), 8.17 (d, J = 6.9 Hz, 1H), 7.85 (d, J = 8.5 Hz, 2H), 7.55-7.43 (m, 5H), 7.07 (d, |
| | J = 4.7 Hz, 1H), 6.20 (dd, J = 17.1, 10.1 Hz, 1H), 6.05 (dd, J = 17.1, 2.3 Hz, 1H), |
| | 5.56 (dd, J = 10.1, 2.3 Hz, 1H), 4.64-4.53 (m, 2H), 4.22-4.14 (m, 1H), 3.15 (dd, J = 10.1, |
| | 6.9 Hz, 1H), 2.85 (dd, J = 16.7, 7.4 Hz, 1H), 2.79-2.69 (m, 2H), 1.98-1.90 (m, 1H), |
| | 1.56-1.49 (m, 1H), 1.30 (s, 9H). |
| 102 | ¹H NMR (400 Mz, DMSO) δ 12.84 (s, 1H), 9.50 (t, J = 5.9 Hz, 1H), 8.46 (d, J = 4.7 Hz, |
| | 1H), 8.15 (d, J = 7.0 Hz, 1H), 7.55-7.45 (m, 3H), 7.08 (d, J = 4.7 Hz, 1H), 6.19 (dd, J = 17.1, |
| | 10.1 Hz, 1H), 6.04 (dd, J = 17.1, 2.3 Hz, 1H), 5.55 (dd, J = 10.1, 2.3 Hz, 1H), |
| | 4.62-4.53 (m, 2H), 4.20-4.13 (m, 1H), 3.12 (dd, J = 10.1, 6.9 Hz, 1H), 2.88-2.81 (m, |
| | 1H), 2.77-2.68 (m, 2H), 1.97-1.91 (m, 1H), 1.57-1.49 (m, 1H), 1.42 (s, 9H). |
| 103 | ¹H NMR (400 Mz, DMSO) δ 12.40 (s, 1H), 9.06 (s, 1H), 8.41 (d, J = 4.7 Hz, 1H), |
| | 7.86 (d, J = 8.3 Hz, 2H), 7.58-7.36 (m, 5H), 6.94 (d, J = 4.7 Hz, 1H), 4.60 (d, J = 5.7 Hz, |
| | 2H), 3.90 (d, J = 7.3 Hz, 1H), 3.42 (s, 1H), 2.80 (d, J = 12.6 Hz, 2H), 2.44 (d, J = 10.9 Hz, |
| | 2H), 1.84 (d, J = 9.9 Hz, 2H), 1.31 (s, 9H), 1.14 (d, J = 9.4 Hz, 2H). |
| 104 | ¹H NMR (400 MHz, DMSO) δ 12.47 (s, 1H), 9.04 (t, J = 5.8 Hz, 1H), 8.42 (d, J = 4.7 Hz, |
| | 1H), 7.85 (d, J = 8.5 Hz, 2H), 7.66-7.33 (m, 5H), 6.96 (d, J = 4.7 Hz, 1H), |
| | 6.75 (dd, J = 16.7, 10.5 Hz, 1H), 6.07 (dd, J = 16.7, 2.4 Hz, 1H), 5.64 (dd, J = 10.4, 2.4 Hz, |
| | 1H), 4.59 (d, J = 5.7 Hz, 2H), 4.16 (d, J = 6.8 Hz, 1H), 4.06 (d, J = 12.2 Hz, 1H), |
| | 3.83 (d, J = 14.4 Hz, 1H), 3.62 (s, 1H), 3.20-3.13 (m, 1H), 2.98-2.88 (m, 1H), 1.91 (s, |
| | 2H), 1.46-1.09 (m, 11H). |
| 105 | ¹H NMR (400 MHz, DMSO) δ 12.48 (s, 1H), 9.53 (t, J = 6.0 Hz, 1H), 8.42 (d, J = 4.7 Hz, |
| | 1H), 7.55-7.44 (m, 3H), 6.97 (d, J = 4.7 Hz, 1H), 6.78 (dd, J = 16.7, 10.5 Hz, 1H), |
| | 6.06 (dd, J = 16.7, 2.5 Hz, 1H), 5.64 (dd, J = 10.4, 2.4 Hz, 1H), 4.58 (d, J = 6.0 Hz, 2H), |
| | 4.19 (d, J = 6.9 Hz, 1H), 4.05 (d, J = 12.9 Hz, 1H), 3.85 (d, J = 13.1 Hz, 1H), 3.62 (d, J = 6.2 Hz, |
| | 1H), 3.21-3.14 (m, 1H), 2.95-2.88 (m, 1H), 1.89 (s, 2H), 1.41 (s, 9H), |
| | 1.33-1.25 (m, 2H). |
| 106 | ¹H NMR (400 MHz, DMSO) δ 9.07 (t, J = 5.9 Hz, 1H), 8.43 (d, J = 4.5 Hz, 1H), 8.35 (t, |
| | J = 5.9 Hz, 1H), 7.88 (d, J = 8.5 Hz, 2H), 7.64-7.39 (m, 5H), 6.93 (d, J = 4.5 Hz, 1H), |
| | 6.27-6.15 (m, 2H), 6.09 (dd, J = 17.1, 2.3 Hz, 1H), 5.57 (dd, J = 10.0, 2.3 Hz, 1H), |
| | 4.68-4.54 (m, 3H), 4.45 (s, 1H), 4.16 (dd, J = 11.1, 7.2 Hz, 1H), 3.55 (dt, J = 13.4, 5.7 Hz, |
| | 1H), 3.38-3.33 (m, 1H), 1.31 (s, 9H). |
| 107 | ¹H NMR (400 MHz, DMSO) δ 9.57 (t, J = 6.0 Hz, 1H), 8.44 (d, J = 4.4 Hz, 1H), 8.37 (t, |
| | J = 5.7 Hz, 1H), 7.60-7.48 (m, 3H), 6.94 (d, J = 4.5 Hz, 1H), 6.29-6.17 (m, 2H), |
| | 6.10 (dd, J = 17.1, 2.1 Hz, 1H), 5.59 (dd, J = 10.0, 2.1 Hz, 1H), 4.67-4.53 (m, 3H), 4.46 (s, |
| | 1H), 4.17 (dd, J = 11.1, 7.2 Hz, 1H), 3.56 (dt, J = 11.3, 5.6 Hz, 1H), 3.41-3.34 (m, 1H), |
| | 1.44 (s, 9H). |
| 108 | ¹H NMR (400 MHz, DMSO) δ 12.98 (s, 1H), 9.56 (t, J = 5.9 Hz, 1H), 8.50 (d, J = 4.7 Hz, |
| | 1H), 8.11 (br, 3H), 7.58-7.48 (m, 3H), 7.12 (d, J = 4.7 Hz, 1H), 4.59 (d, J = 5.9 Hz, |
| | 2H), 3.67-3.64 (m, 1H), 3.28 (dd, J = 10.8, 7.2 Hz, 1H), 2.98 (dd, J = 10.9, 5.4 Hz, |
| | 1H), 2.81 (dd, J = 16.6, 7.3 Hz, 1H), 2.70-2.64 (m, 1H), 2.03-1.96 (m, 1H), |
| | 1.69-1.62 (m, 1H), 1.43 (s, 9H). |
| 109 | ¹H NMR (400 MHz, DMSO) δ 12.59 (s, 1H), 9.56 (t, J = 6.0 Hz, 1H), 8.74 (s, 1H), |
| | 8.52 (s, 1H), 8.45 (d, J = 4.8 Hz, 1H), 7.58-7.46 (m, 3H), 7.01 (d, J = 4.8 Hz, 1H), 4.59 (d, J = 6.0 Hz, |
| | 2H), 4.36 (s, 1H), 3.21-3.14 (m, 2H), 2.94 (d, J = 10.8 Hz, 2H), 2.06 (d, J = 10.4 Hz, |
| | 2H), 1.60 (dd, J = 19.6, 9.8 Hz, 2H), 1.44 (s, 9H). |
| 121 | ¹H NMR (400 MHz, DMSO) δ 12.64 (s, 1H), 9.72 (s, 1H), 8.90 (br, 1H), 8.76 (br, 1H), |
| | 8.45 (d, J = 4.8 Hz, 1H), 7.74 (t, J = 8.0 Hz, 1H), 7.55-7.47 (m, 2H), 7.02 (d, J = 4.8 Hz, |
| | 1H), 4.39 (s, 1H), 3.75 (s, 1H), 3.35 (d, J = 11.7 Hz, 1H), 3.07 (s, 1H), |
| | 2.97-2.90 (m, 1H), 2.81 (d, J = 9.6 Hz, 1H), 1.88 (s, 1H), 1.72 (s, 1H), 1.65-1.57 (m, 1H), |
| | 1.46-1.38 (m, 10H), 1.34-1.29 (m, 4H). |
| 122 | ¹H NMR (400 MHz, DMSO) δ 12.62 (s, 1H), 9.09 (s, 1H), 9.02 (br, 2H), 8.45 (d, J = 4.8 Hz, |
| | 1H), 7.63 (d, J = 8.4 Hz, 2H), 7.58 (d, J = 8.4 Hz, 2H), 7.02 (d, J = 4.8 Hz, 1H), |
| | 4.15 (s, 2H), 3.34-3.27 (m, 1H), 3.24-3.13 (m, 3H), 2.16-2.10 (m, 1H), 1.82-1.64 (m, |
| | 7H), 1.43 (s, 9H). |
| 123 | ¹H NMR (400 MHz, DMSO) δ 12.71 (s, 1H), 11.47 (s, 1H), 9.20 (br, 2H), 8.47 (d, J = 4.8 Hz, |
| | 1H), 7.62-7.48 (m, 3H), 7.07 (d, J = 4.8 Hz, 1H), 4.68 (br, 1H), 4.25-4.19 (m, |
| | 1H), 3.91 (s, 2H), 3.42-3.33 (m, 1H), 3.28-3.14 (m, 3H), 2.22-2.13 (m, 1H), |
| | 1.88-1.80 (m, 1H), 1.39 (s, 9H). |
| 124 | ¹H NMR (400 MHz, DMSO) δ 12.72 (s, 1H), 9.57 (t, J = 6.0 Hz, 1H), 9.11 (s, 1H), |
| | 8.89 (s, 1H), 8.46 (d, J = 4.7 Hz, 1H), 7.59-7.47 (m, 3H), 7.04 (d, J = 4.8 Hz, 1H), 5.50 (s, |
| | 1H), 4.61 (d, J = 6.0 Hz, 2H), 4.54-4.44 (m, 1H), 4.16-4.07 (m, 2H), 3.87-3.81 (m, |
| | 2H), 1.44 (s, 9H). |
| 129 | ¹H NMR (400 MHz, DMSO) δ 12.72 (s, 1H), 9.61 (t, J = 6.1 Hz, 1H), 9.30 (br, 2H), |
| | 8.46 (d, J = 4.8 Hz, 1H), 7.59 (t, J = 8.0 Hz, 1H), 7.34 (dd, J = 8.9, 4.7 Hz, 2H), 7.01 (d, |
| | J = 3.5 Hz, 1H), 4.56 (d, J = 6.1 Hz, 2H), 4.18 (s, 1H), 3.38-3.28 (m, 1H), |
| | 3.23-3.04 (m, 3H), 2.19-2.08 (m, 1H), 1.78-1.66 (m, 1H). |
| 130 | ¹H NMR (400 MHz, DMSO) δ 12.59 (s, 1H), 9.54 (t, J = 6.0 Hz, 1H), 8.44 (d, J = 4.7 Hz, |
| | 1H), 7.56-7.46 (m, 3H), 7.01 (d, J = 4.7 Hz, 1H), 5.28 (d, J = 6.7 Hz, 1H), 4.60 (d, |
| | J = 6.0 Hz, 2H), 4.36-4.27 (m, 2H), 4.09-4.02 (m, 1H), 3.81 (t, J = 6.4 Hz, 1H), |
| | 3.59 (dd, J = 9.6, 4.3 Hz, 1H), 2.06-1.96 (m, 2H), 1.44 (s, 9H), 0.93 (t, J = 7.5 Hz, 3H). |
| 131 | ¹H NMR (400 MHz, DMSO) δ 12.61 (s, 1H), 9.54 (t, J = 6.0 Hz, 1H), 8.44 (d, J = 4.7 Hz, |
| | 1H), 7.56-7.44 (m, 3H), 7.01 (d, J = 4.7 Hz, 1H), 5.41 (d, J = 7.1 Hz, 1H), 4.60 (d, |
| | J = 6.0 Hz, 2H), 4.40-4.30 (m, 2H), 4.15-4.08 (m, 1H), 3.92-3.86 (m, 1H), 3.64 (dd, |
| | J = 10.1, 4.1 Hz, 1H), 1.97 (s, 3H), 1.44 (s, 9H). |
| 150 | ¹H NMR (400 MHz, DMSO) δ 12.62 (s, 1H), 10.42 (d, J = 16.7 Hz, 1H), 9.07 (br, 2H), |
| | 8.47 (dd, J = 4.7, 1.3 Hz, 1H), 8.15-8.06 (m, 1H), 7.99-7.86 (m, 2H), 7.59 (d, J = 8.0 Hz, |
| | 2H), 7.49 (td, J = 7.9, 3.0 Hz, 1H), 7.13 (ddd, J = 22.5, 7.6, 1.1 Hz, 1H), 6.94 (dd, J = 5.7, |
| | 4.8 Hz, 1H), 4.47-4.40 (m, 2H), 4.20-4.12 (m, 1H), 3.41-3.30 (m, 1H), |
| | 3.19-2.99 (m, 3H), 2.20-2.10 (m, 1H), 1.74-1.56 (m, 1H), 1.33 (s, 9H). |
| 151 | ¹H NMR (400 MHz, DMSO) δ 12.50 (s, 1H), 9.55 (t, J = 6.1 Hz, 1H), 8.43 (d, J = 4.7 Hz, |
| | 1H), 7.87 (br, 3H), 7.53 (t, J = 7.8 Hz, 1H), 7.50-7.42 (m, 2H), 6.96 (d, J = 4.8 Hz, |
| | 1H), 4.59 (d, J = 6.1 Hz, 2H), 3.30 (s, 1H), 2.95 (s, 1H), 2.06 (d, J = 10.6 Hz, 2H), |
| | 1.91 (d, J = 10.5 Hz, 2H), 1.44 (s, 9H), 1.38-1.31 (m, 2H), 1.17-1.09 (m, 2H). |
| 152 | ¹H NMR (400 MHz, DMSO) δ 12.43 (s, 1H), 9.54 (s, 1H), 8.42 (d, J = 4.7 Hz, 1H), |
| | 7.93 (d, J = 7.8 Hz, 1H), 7.55 (d, J = 7.9 Hz, 1H), 7.51-7.40 (m, 2H), 6.96 (d, J = 4.7 Hz, |
| | 1H), 6.16 (d, J = 10.1 Hz, 1H), 6.07 (d, J = 2.3 Hz, 1H), 5.54 (dd, J = 10.0, 2.4 Hz, 1H), |
| | 4.60 (d, J = 6.0 Hz, 2H), 3.94 (d, J = 7.4 Hz, 1H), 3.58-3.50 (m, 1H), 3.30-3.24 (m, |
| | 1H), 2.00 (d, J = 11.0 Hz, 2H), 1.76 (d, J = 10.8 Hz, 2H), 1.43 (s, 9H), 1.24-1.09 (m, |
| | 4H). |
| 153 | ¹H NMR (400 MHz, DMSO) δ 12.53 (s, 1H), 9.58 (t, J = 6.0 Hz, 1H), 8.44 (d, J = 4.8 Hz, |
| | 1H), 7.87 (br, 3H), 7.67 (t, J = 7.9 Hz, 1H), 7.56-7.47 (m, 2H), 6.99 (d, J = 4.8 Hz, |
| | 1H), 4.59 (d, J = 5.9 Hz, 2H), 4.33-3.70 (m, 1H), 3.63 (s, 1H), 3.09 (s, 1H), |
| | 1.87-1.78 (m, 2H), 1.72-1.59 (m, 4H), 1.54-1.46 (m, 2H), 1.44 (s, 9H). |
| 154 | ¹H NMR (400 MHz, DMSO) δ 12.42 (s, 1H), 9.59 (t, J = 6.0 Hz, 1H), 8.42 (d, J = 4.7 Hz, |
| | 1H), 7.80 (d, J = 7.4 Hz, 1H), 7.62 (t, J = 7.8 Hz, 1H), 7.53-7.45 (m, 2H), 6.96 (d, |
| | J = 4.7 Hz, 1H), 6.28 (dd, J = 17.1, 10.1 Hz, 1H), 6.05 (dd, J = 17.1, 2.3 Hz, 1H), |
| | 5.53 (dd, J = 10.1, 2.3 Hz, 1H), 4.59 (d, J = 6.0 Hz, 2H), 3.88 (d, J = 5.9 Hz, 1H), |
| | 3.73-3.68 (m, 1H), 3.64-3.59 (m, 1H), 1.69-1.60 (m, 4H), 1.57-1.50 (m, 2H), 1.42 (s, 9H), |
| | 1.37-1.30 (m, 2H). |
| 155 | ¹H NMR (400 MHz, DMSO) δ 12.66 (s, 1H), 10.60 (s, 1H), 9.60 (d, J = 2.4 Hz, 1H), |
| | 9.05 (br, 2H), 8.45 (d, J = 4.8 Hz, 1H), 8.35 (t, J = 8.4 Hz, 1H), 7.69 (dd, J = 12.2, 1.8 Hz, |
| | 1H), 7.57 (d, J = 8.4 Hz, 1H), 7.05 (d, J = 4.8 Hz, 1H), 4.91 (s, 1H), 4.25 (s, 1H), |
| | 3.41-3.34 (m, 1H), 3.30-3.20 (m, 3H), 2.22-2.14 (m, 1H), 1.95-1.87 (m, 1H), |
| | 1.40 (s, 9H). |
| 156 | ¹H NMR (400 MHz, DMSO) δ 12.55 (s, 1H), 10.48 (d, J = 3.6 Hz, 1H), 9.53-9.46 (m, |
| | 1H), 8.42 (d, J = 4.7 Hz, 1H), 8.32 (t, J = 8.4 Hz, 1H), 7.60 (d, J = 12.3 Hz, 1H), |
| | 7.49-7.44 (m, 1H), 7.00 (d, J = 4.8 Hz, 1H), 6.62-6.46 (m, 1H), 6.11 (ddd, J = 16.8, 9.6, 2.4 Hz, |
| | 1H), 5.63 (ddd, J = 20.1, 10.3, 2.4 Hz, 1H), 4.66 (d, J = 4.9 Hz, 1H), 4.24-4.13 (m, |
| | 1H), 3.89-3.59 (m, 2H), 3.49-3.37 (m, 2H), 2.20-2.09 (m, 1H), 1.96-1.83 (m, 1H), |
| | 1.41 (s, 9H). |
| 160 | ¹H NMR (400 MHz, DMSO) δ 12.54 (d, J = 3.1 Hz, 1H), 10.88 (s, 1H), 8.48-8.40 (m, |
| | 1H), 8.17 (d, J = 8.2 Hz, 1H), 7.52-7.44 (m, 1H), 7.17-7.06 (m, 1H), 6.94-6.87 (m, |
| | 1H), 6.53-6.39 (m, 1H), 6.10-6.02 (m, z, 1H), 5.90-5.77 (m, 1H), 5.60-5.46 (m, |
| | 1H), 4.49-4.33 (m, 2H), 4.11-4.00 (m, 1H), 3.95-3.83 (m, 1H), 3.81-3.70 (m, 1H), |
| | 3.56-3.48 (m, 1H), 3.41-3.35 (m, 1H), 3.26-3.19 (m, 1H), 2.12-1.99 (m, 1H), |
| | 1.72-1.58 (m, 1H), 1.45 (s, 9H). |
| 161 | ¹H NMR (400 MHz, DMSO) δ 12.51 (d, J = 2.5 Hz, 1H), 10.35-10.23 (m, 1H), |
| | 8.50-8.39 (m, 1H), 8.16 (dd, J = 15.2, 8.0 Hz, 1H), 7.95-7.81 (m, 2H), 7.59 (dd, J = 8.5, 1.8 Hz, |
| | 2H), 7.46 (ddd, J = 10.2, 8.0, 2.2 Hz, 1H), 7.06 (dd, J = 27.1, 7.6 Hz, 1H), |
| | 6.96-6.86 (m, 1H), 6.55-6.32 (m, 1H), 6.04 (ddd, J = 26.4, 16.7, 2.4 Hz, 1H), |
| | 5.85-5.68 (m, 1H), 5.60-5.34 (m, 1H), 4.50-4.31 (m, 2H), 4.14-4.01 (m, 1H), 3.98-3.65 (m, |
| | 2H), 3.54-3.47 (m, 1H), 2.13-1.98 (m, 1H), 1.75-1.61 (m, 1H), 1.33 (s, 9H). |
| 162 | ¹H NMR (400 MHz, DMSO) δ 12.70 (s, 1H), 9.95 (t, J = 6.0 Hz, 1H), 9.24 (br, 2H), |
| | 8.47 (d, J = 4.8 Hz, 1H), 7.68-7.44 (m, 3H), 7.04 (d, J = 4.8 Hz, 1H), 4.60 (d, J = 5.9 Hz, |
| | 2H), 4.24-4.20 (m, 1H), 3.40-3.30 (m, 1H), 3.29-3.10 (m, 3H), 2.23-2.11 (m, |
| | 1H), 1.89-1.77 (m, 1H), 1.37 (s, 9H). |
| 163 | ¹H NMR (400 MHz, DMSO) δ 12.74 (s, 1H), 11.13-10.90 (m, 1H), 10.03-9.87 (m, |
| | 1H), 8.47 (d, J = 4.8 Hz, 1H), 7.69-7.42 (m, 3H), 7.05 (dd, J = 4.8, 2.0 Hz, 1H), |
| | 4.59 (s, 2H), 4.33-4.22 (m, 1H), 3.76-3.50 (m, 1H), 3.48-3.13 (m, 3H), 3.11-2.97 (m, |
| | 1H), 2.44-2.12 (m, 1H), 2.04-1.71 (m, 1H), 1.37 (d, J = 1.2 Hz, 9H), 1.33-1.16 (m, |
| | 6H). |
| 164 | ¹H NMR (400 MHz, DMSO) δ 12.57 (s, 1H), 9.93 (dd, J = 9.6, 5.9 Hz, 1H), 8.44 (dd, J = 4.7, |
| | 1.9 Hz, 1H), 7.59-7.40 (m, 3H), 7.00 (dd, J = 4.7, 2.6 Hz, 1H), 4.59 (d, J = 5.9 Hz, |
| | 2H), 4.52-4.44 (m, 1H), 4.21-4.07 (m, 1H), 3.72-3.43 (m, 2H), 3.30-3.22 (m, |
| | 2H), 2.17-2.01 (m, 1H), 1.94-1.72 (m, 4H), 1.37 (s, 9H). |
| 165 | ¹H NMR (400 MHz, DMSO) δ 12.64 (s, 1H), 10.93 (d, J = 19.2 Hz, 1H), 8.95 (s, 2H), |
| | 8.47 (dd, J = 4.7, 1.4 Hz, 1H), 8.16 (dd, J = 8.0, 3.0 Hz, 1H), 7.55-7.48 (m, 1H), |
| | 7.19 (dd, J = 22.8, 7.6 Hz, 1H), 6.95-6.89 (m, 1H), 4.50-4.41 (m, 2H), 4.15-4.09 (m, |
| | 1H), 3.36-3.29 (m, 1H), 3.18-2.93 (m, 4H), 2.16-2.09 (m, 1H), 1.67-1.59 (m, 1H), |
| | 11.45 (s, 9H). |
| 166 | ¹H NMR (400 MHz, DMSO) δ 12.70 (s, 1H), 10.51 (d, J = 78.8 Hz, 1H), 9.94 (s, 1H), |
| | 8.46 (d, J = 4.7 Hz, 1H), 7.64-7.45 (m, 3H), 7.10-6.99 (m, 1H), 4.90 (d, J = 53.1 Hz, |
| | 1H), 4.60 (d, J = 5.8 Hz, 2H), 4.28 (d, J = 43.5 Hz, 1H), 3.88-3.78 (m, 1H), |
| | 3.56-3.52 (m, 1H), 3.25-3.14 (m, 1H), 3.04-2.92 (m, 1H), 2.80 (t, J = 5.0 Hz, 3H), |
| | 2.25-1.71 (m, 2H), 1.37 (s, 9H). |
| 167 | ¹H NMR (400 MHz, DMSO) δ 12.65 (s, 1H), 9.95 (t, J = 5.9 Hz, 1H), 8.93 (s, 1H), |
| | 8.81 (s, 1H), 8.46 (d, J = 4.7 Hz, 1H), 7.64-7.48 (m, 3H), 7.02 (d, J = 4.8 Hz, 1H), 4.60 (d, J = 5.8 Hz, |
| | 2H), 4.43 (s, 1H), 3.81 (s, 1H), 3.38 (d, J = 11.1 Hz, 1H), 3.15-3.07 (m, 1H), |
| | 2.97-2.89 (m, 1H), 2.86-2.78 (m, 1H), 1.97-1.89 (m, 1H), 1.81-1.73 (m, 1H), |
| | 1.69-1.58 (m, 1H), 1.50-1.26 (m, 10H). |
| 168 | ¹H NMR (400 MHz, DMSO) δ 12.50 (d, J = 21.4 Hz, 1H), 10.00-9.90 (m, 1H), 8.43 (t, |
| | J = 5.3 Hz, 1H), 7.64-7.53 (m, 1H), 7.44 (t, J = 12.1 Hz, 1H), 7.37 (d, J = 7.7 Hz, 1H), |
| | 6.96 (dd, J = 9.9, 4.7 Hz, 1H), 4.61 (d, J = 5.9 Hz, 2H), 4.04 (dd, J = 25.7, 7.2 Hz, 1H), |
| | 3.71-3.53 (m, 2H), 3.51-3.35 (m, 2H), 1.97-1.71 (m, 4H), 1.53-1.29 (m, 12H). |
| 169 | ¹H NMR (400 MHz, DMSO) δ 12.51 (s, 1H), 9.92 (s, 1H), 8.42 (d, J = 4.7 Hz, 1H), |
| | 7.55 (t, J = 7.9 Hz, 1H), 7.43 (d, J = 10.7 Hz, 1H), 7.33 (d, J = 7.2 Hz, 1H), 6.96 (d, J = 4.6 Hz, |
| | 1H), 6.84-6.33 (m, 1H), 5.99 (t, J = 16.1 Hz, 1H), 5.53 (dd, J = 72.0, 11.2 Hz, 1H), |
| | 4.58 (d, J = 5.7 Hz, 2H), 4.06 (d, J = 7.0 Hz, 1H), 3.80-3.36 (m, 5H), 1.91-1.77 (m, |
| | 1H), 1.58-1.42 (m, 2H), 1.37-1.35 (m, 10H). |
| 170 | ¹H NMR (400 MHz, DMSO) δ 12.61 (br, 1H), 9.49-9.32 (m, 2H), 8.89 (s, 1H), |
| | 8.48 (d, J = 4.9 Hz, 1H), 7.61-7.47 (m, 3H), 7.05 (d, J = 4.9 Hz, 1H), 6.64 (s, 1H), 4.57 (d, J = 5.9 Hz, |
| | 2H), 4.49 (s, 1H), 4.03 (s, 1H), 3.63 (s, 1H), 3.01-2.78 (m, 4H), |
| | 1.81-1.51 (m, 4H), 1.25 (t, J = 7.6 Hz, 3H), 1.16 (d, J = 6.8 Hz, 3H). |
| 171 | ¹H NMR (400 MHz, DMSO) δ 12.47 (s, 1H), 9.36 (t, J = 6.0 Hz, 1H), 8.43 (d, J = 4.7 Hz, |
| | 1H), 7.55 (t, J = 7.8 Hz, 1H), 7.47 (dd, J = 10.8, 1.3 Hz, 1H), 7.42 (dd, J = 7.8, 1.4 Hz, |
| | 1H), 6.96 (d, J = 4.7 Hz, 1H), 6.60 (s, 1H), 4.56 (d, J = 5.9 Hz, 2H), 4.09 (dd, J = 12.2, |
| | 6.0 Hz, 1H), 3.82 (d, J = 7.2 Hz, 1H), 3.64-3.57 (m, 1H), 3.18 (d, J = 13.1 Hz, |
| | 1H), 2.82 (q, J = 7.6 Hz, 2H), 2.75 (t, J = 12.1 Hz, 1H), 2.69 (s, 6H), 1.73-1.64 (m, |
| | 1H), 1.53 (d, J = 12.8 Hz, 1H), 1.47-1.37 (m, 1H), 1.34-1.27 (m, 1H), 1.24 (t, J = 7.6 Hz, |
| | 3H), 0.84 (d, J = 6.8 Hz, 3H). |
| 172 | ¹H NMR (400 MHz, DMSO) δ 12.61 (s, 1H), 9.37 (t, J = 6.0 Hz, 1H), 9.16 (s, 1H), |
| | 8.68 (s, 1H), 8.45 (d, J = 4.8 Hz, 1H), 7.59-7.43 (m, 3H), 7.01 (d, J = 4.8 Hz, 1H), 6.62 (d, J = 0.7 Hz, |
| | 1H), 4.57 (d, J = 5.9 Hz, 2H), 4.33 (s, 1H), 4.00 (s, 1H), 3.65 (s, 1H), 3.16 (dt, |
| | J = 13.9, 6.9 Hz, 1H), 2.92 (s, 2H), 1.76-1.52 (m, 4H), 1.28 (d, J = 6.9 Hz, 6H), |
| | 1.15 (d, J = 6.8 Hz, 3H). |
| 173 | ¹H NMR (400 MHz, DMSO) δ 12.51 (s, 1H), 9.35 (t, J = 6.0 Hz, 1H), 8.43 (d, J = 4.7 Hz, |
| | 1H), 7.55 (t, J = 7.8 Hz, 1H), 7.49 (dd, J = 10.8, 1.5 Hz, 1H), 7.43 (dd, J = 7.8, 1.5 Hz, |
| | 1H), 6.98 (d, J = 4.7 Hz, 1H), 6.72 (dd, J = 16.4, 10.4 Hz, 1H), 6.59 (d, J = 0.8 Hz, |
| | 1H), 6.06 (d, J = 16.9 Hz, 1H), 5.64 (d, J = 10.4 Hz, 1H), 4.85 (d, J = 133.7 Hz, 1H), |
| | 4.57 (d, J = 6.0 Hz, 2H), 4.25-3.82 (m, 2H), 3.54 (s, 1H), 3.15 (td, J = 13.3, 7.0 Hz, |
| | 1H), 2.93-2.61 (m, 1H), 1.68 (dd, J = 46.2, 7.5 Hz, 2H), 1.35 (s, 2H), 1.27 (d, J = 6.9 Hz, |
| | 6H), 0.88 (s, 3H). |
| 174 | ¹H NMR (400 MHz, DMSO) δ 12.67 (s, 1H), 9.58 (t, J = 6.1 Hz, 1H), 9.23 (s, 2H), |
| | 8.45 (d, J = 4.8 Hz, 1H), 7.69 (d, J = 8.1 Hz, 2H), 7.51 (d, J = 8.1 Hz, 2H), 7.00 (d, J = 4.8 Hz, |
| | 1H), 4.56 (d, J = 6.1 Hz, 2H), 4.21 (d, J = 4.4 Hz, 1H), 3.42-3.31 (m, 1H), |
| | 3.29-3.08 (m, 3H), 2.18 (td, J = 14.4, 7.5 Hz, 1H), 1.78 (dt, J = 12.7, 6.7 Hz, 1H), 1.43 (s, |
| | 9H). |
| 175 | ¹H NMR (400 MHz, DMSO) δ 12.52 (s, 1H), 9.54 (dd, J = 11.0, 6.2 Hz, 1H), |
| | 8.44-8.40 (m, 1H), 7.56 (d, J = 8.1 Hz, 2H), 7.47 (dd, J = 8.2, 4.2 Hz, 2H), 6.94 (dd, J = 4.7, |
| | 2.7 Hz, 1H), 6.56-6.46 (m, 1H), 6.12 (ddd, J = 16.8, 5.9, 2.4 Hz, 1H), 5.66-5.58 (m, |
| | 1H), 4.53 (d, J = 5.8 Hz, 2H), 4.26-4.11 (m, 2H), 3.83-3.42 (m, 4H), 2.16-2.07 (m, |
| | 1H), 1.83-1.73 (m, 1H), 1.43 (s, 9H). |

### Example 62a: Btk In Vitro Inhibitory Activity (Method A)

The Btk IC₅₀s of compounds disclosed herein is determined in both a cellular kinase assay and in a cellular functional assay of BCR-induced calcium flux as described below.

Btk kinase activity is determined using a time-resolved fluorescence resonance energy transfer (TR-FRET) methodology. Measurements are performed in a reaction volume of 50 μL using 96-well assay plates. Kinase enzyme, inhibitor, ATP (at the Kₘ for the kinase), and 1 μM peptide substrate (Biotin-AVLESEEELYSSARQ-NH₂) are incubated in a reaction buffer composed of 20 mM Tris, 50 mM NaCl, MgCl₂ (5-25 mM depending on the kinase), MnCl₂ (0-10 mM), 1 mM DTT, 0.1 mM EDTA, 0.01% bovine serum albumin, 0.005% Tween-20, and 10% DMSO at pH 7.4 for one hour. The reaction is quenched by the addition of 1.2 equivalents of EDTA (relative to divalent cation) in 25 μL of 1× Lance buffer (Perkin-Elmer). Streptavidin-APC (Perkin-Elmer) and Eu-labeled p-Tyr100 antibody (Perkin-Elmer) in 1× Lance buffer are added in a 25 μL volume to give final concentrations of 100 nM and 2.5 nM, respectively, and the mixture is allowed to incubate for one hour. The TR-FRET signal is measured on a multimode plate reader with an excitation wavelength (λ_{Ex}) of 330 nm and detection wavelengths (λ_{Em}) of 615 and 665 nm. Activity is determined by the ratio of the fluorescence at 665 nm to that at 615 nm. For each compound, enzyme activity is measured at various concentrations of compound. Negative control reactions are performed in the absence of inhibitor in replicates of six, and two no-enzyme controls are used to determine baseline fluorescence levels. Inhibition constants, Kᵢ(app), were obtained using the program BatchKᵢ (Kuzmic et al. (2000), Anal. Biochem. 286:45-50). IC₅₀s are obtained according to the equation:

IC₅₀={Ki(app)/(1+[ATP]/Kₘ^{ATP})}+[E]ₜₒₜₐl/2;

For all kinases, [ATP]=Kₘ^{ATP}, [Btk]ₜₒₜₐl=0.5 nM and [Lck]ₜₒₜₐl=6 nM.

### Example 62b: Btk In Vitro Inhibitory Activity (Method B)

Kinase activity is measured in vitro using electrophoretic mobility shift assay. The kinase reactions are assembled in a total volume of 25 μL in 384 well plates. The reactions comprise: BTK enzyme (1 nM, N-terminal His6-tagged, recombinant, full-length, human BTK purified from baculovirus Sf21 insect cell system, 293HEK, or other suitable source), inhibitor, ATP, fluorescently labeled peptide substrate (1 μM, FAM-GEEPLYWSFPAKKK-NH₂) in a reaction buffer composed of 100 mM HEPES, pH7.5, 5 mM MgCl₂ 1 mM DTT, 0.1% bovine serum albumin, 0.01% Triton X-100, and 1% DMSO. The reaction is incubated and is quenched by the addition of termination buffer (100 mM HEPES, pH 7.5, 0.01% Triton X-100, 30 mM EDTA). The terminated reactions are analyzed using a 12 channel LabChip® 3000 microfluidic detection instrument (Caliper Life Sciences). The enzymatic phosphorylation of the peptide results in a change in net charge, enabling electrophoretic separation of product from substrate peptide. As substrate and product peptides are separated, two peaks of fluorescence are observed. Change in the relative fluorescence intensity of the substrate and product peaks is the parameter measured, reflecting enzyme activity. In the presence of an inhibitor, the ratio between product and substrate is altered: the signal of the product decreases, while the signal of the substrate increases.

Activity in each sample is determined as the product to sum ratio (PSR):P/(S+P), where P is the peak height of the product peptide and S is the peak height of the substrate peptide. For each compound, enzyme activity is measured at various concentrations (12 concentrations of compound spaced by 3× dilution intervals). Negative control samples (0%—inhibition in the absence of inhibitor) and positive control samples (100%—inhibition, in the presence of 20 mM EDTA) are assembled in replicates of four and are used to calculate %—inhibition values for each inhibitor at each concentration. Percent inhibition (Pᵢₙₕ) is determined using following equation:

Pᵢₙₕ=(PSR_{0%}−PSRᵢₙₕ)/(PSR_{0%}−PSR_{100%})*100, where PSRᵢₙₕ is the product sum ratio in the presence of inhibitor, PSR_{0%} is the average product sum ration in the absence of inhibitor and PSR_{100%} is the average product sum ratio in 100%—inhibition control samples.

The IC₅₀ values of inhibitors are determined by 4 parameter sigmoidal dose-response model fitting of the inhibition curves (Pᵢₙₕ versus inhibitor concentration) using XLfit 4 software.

### Example 62c: Btk In Vitro Inhibitory Activity (Method C)

Human Btk kinase (Genbank accession # NP_000052) was purified from insect cells as a full-length construct containing a N-terminal 6X-His tag. Btk kinase activity was determined using a radiometric filter binding assay. Measurements were performed in a low μL reaction volume 384-well assay plates. BTK enzyme (8 nM final in reaction), inhibitor (at requested doses), and 0.2 mg/mL peptide substrate (Poly-Glu-Tyr, 4:1 ratio) were incubated in a reaction buffer composed of 20 mM Hepes (pH 7.5), 10 mM MgCl₂, 1 mM EGTA, 0.02% Brij35, 0.02 mg/mL BSA, 0.1 mM Na₃VO₄, 2 mM DTT, 1% DMSO for 15 min. followed by addition of 1 μM ATP to start the assay. Kinase reactions are carried out for 120 min. at room temperature. The reaction was stopped by spotting of reaction sample onto P81 cationic exchange paper (Whatman). Unbound phosphate was removed by extensive washing of filters in 0.75% Phosphoric acid. After subtraction of background derived from control reactions containing inactive enzyme (via addition of saturating EDTA), kinase activity data for each dose of compound tested was expressed as the percent of remaining kinase activity in test samples compared to vehicle (dimethyl sulfoxide) reactions. IC₅₀ values and curve fits were obtained using Prism (GraphPad Software).

The degree of Btk inhibition of exemplifying compounds was determined using one of the methods outlined in Example 61a, 61b and 61c.

### Example 63: Inhibition of a Panel of Kinases

The degree of inhibition of a panel of kinases is determined using the in vitro HotSpot kinase assay (purified enzymes, ³³P-ATP, an appropriate substrate and 1 μM ATP).

TABLE 3
IC₅₀ Values for Exemplary Compounds described herein.

| Compound | Btk-WT IC50 | BMX-WT | TEC IC50 | |
|---|---|---|---|---|
| ID | (nM) | IC50 (nM) | (nM) | TEC/Btk |
| 1 | 57.7 | >10000 | >10000 | >100 |
| 2 | 11.6 | 1980 | 950 | 82 |
| 3 | 0.205 | 39 | 20 | 98 |
| 4 | 0.559 | 132 | 103 | 184 |
| 5 | 52.7 | 4350 | 2590 | 49 |
| 6 | 0.62 | 182 | 130 | 210 |
| 7 | 1.65 | 274 | 296 | 179 |
| 8 | 144 | >10000 | >10000 | — |
| 9 | 3.62 | 1910 | 2460 | 680 |
| 10 | 13.1 | >10000 | 8350 | 637 |
| 11 | 1000 | 2580 | 10000 | 10 |
| 12 | 879 | 10000 | 10000 | 11 |
| 13 | 1000 | 10000 | 10000 | 10 |
| 14 | 1000 | 10000 | 10000 | 10 |
| 15 | 1000 | 10000 | 10000 | 10 |
| 16 | >10000 | 4050 | 3990 | — |
| 17 | 312 | 6410 | 5920 | 19 |
| 18 | 1.69 | 73 | 40 | 23 |
| 19 | >10000 | 7460 | >10000 | — |
| 20 | >10000 | 5280 | >10000 | — |
| 21 | 53.4 | 4370 | 3170 | 59 |
| 22 | >10000 | 6470 | >10000 | — |
| 23 | >10000 | >10000 | >10000 | — |
| 24 | 0.139 | 44 | 22 | 156 |
| 25 | 0.481 | 794 | 175 | 365 |
| 26 | 932 | — | >10000 | >100 |
| 27 | 44.2 | — | 6260 | 142 |
| 28 | 0.575 | 71 | 200 | 347 |
| 29 | 11.0 | 595 | 964 | 88 |
| 30 | 254 | 6750 | 10000 | 39 |
| 31 | >10000 | 1710 | 6410 | — |
| 32 | 328 | 1410 | 5440 | 17 |
| 33 | 15.0 | 25 | 217 | 14 |
| 34 | 1.76 | 16 | 55 | 31 |
| 35 | 505 | 8480 | 7000 | 14 |
| 36 | 433 | >10000 | >10000 | >100 |
| 37 | 53.9 | 8820 | >10000 | >100 |
| 38 | >10000 | >10000 | >10000 | — |
| 39 | 63.6 | 3820 | 4680 | 74 |
| 40 | 1.27 | 54 | 47 | 37 |
| 41 | 251 | >10000 | 3010 | 12 |
| 42 | >10000 | >10000 | >10000 | — |
| 43 | 2.76 | 394 | 599 | 217 |
| 44 | 68.2 | 5390 | 7940 | 116 |
| 45 | >10000 | 449 | 1920 | >100 |
| 46 | 8.55 | 91 | 182 | 21 |
| 47 | 682 | 1830 | 2940 | 4 |
| 48 | 0.798 | 46 | 65 | 81 |
| 49 | 1.78 | 89 | 70 | 39 |
| 50 | 3.2 | 7440 | 2957 | 925 |
| 51 | — | — | — | — |
| 52 | 45.0 | — | 8450 | 188 |
| 53 | 0.623 | 310 | 152 | 244 |
| 54 | >10000 | — | >10000 | — |
| 55 | 29.2 | >10000 | >10000 | >100 |
| 56 | 52.5 | >10000 | >10000 | >100 |
| 57 | 0.0424 | 5 | 6 | 137 |
| 58 | 0.0535 | 10 | 11 | 213 |
| 59 | 7.84 | — | 1110 | 142 |
| 60 | 21.0 | — | 7420 | 353 |
| 61 | 34.1 | — | 5950 | 174 |
| 62 | 861 | 2140 | 3340 | 4 |
| 63 | 10.2 | >10000 | 7860 | 771 |
| 64 | 167 | >10000 | >10000 | >100 |
| 65 | 0.0792 | 78 | 42 | 529 |
| 66 | 0.42 | 219 | 403 | 959 |
| 67 | 0.359 | 137 | 121 | 337 |
| 68 | 0.601 | 541 | 258 | 429 |
| 69 | 10.4 | 6970 | 4510 | 434 |
| 70 | 180 | >10000 | >10000 | >100 |
| 71 | 42.2 | >10000 | >10000 | >100 |
| 72 | >10000 | >10000 | >10000 | >100 |
| 73 | 37.9 | >10000 | >10000 | >100 |
| 74 | >10000 | >10000 | >10000 | — |
| 75 | >10000 | >10000 | >10000 | — |
| 76 | 51.1 | 8360 | 8720 | 171 |
| 77 | 159 | >10000 | >10000 | >100 |
| 78 | >10000 | >10000 | >10000 | — |
| 79 | 6.27 | 1790 | 1730 | 276 |
| 80 | 0.129 | 76 | 57 | 440 |
| 81 | 0.227 | 89 | 131 | 577 |
| 82 | 30.6 | 113 | 357 | 12 |
| 83 | 0.056 | 52 | 28 | 493 |
| 84 | 7.83 | 87 | 361 | 46 |
| 85 | 0.19 | 115 | 110 | 579 |
| 86 | 0.242 | 218 | 213 | 880 |
| 87 | 1.43 | 276 | 685 | 479 |
| 88 | 287 | 713 | 2380 | 8 |
| 89 | >10000 | 510 | 3280 | >100 |
| 90 | 9.44 | 2620 | 2250 | 238 |
| 91 | >10000 | — | >10000 | — |
| 92 | 13.7 | 7600 | 6920 | 505 |
| 93 | 11.7 | 3340 | 3770 | 322 |
| 94 | 25.3 | >10000 | >10000 | >100 |
| 95 | 67.9 | 774 | 924 | 14 |
| 96 | >10000 | — | 9640 | — |
| 97 | 309 | 709 | 1570 | 5 |
| 98 | 0.165 | 239 | 108 | 652 |
| 99 | 4.46 | — | 1430 | 321 |
| 100 | 13.8 | 1890 | 2970 | 215 |
| 101 | 0.22 | 122 | 96 | 434 |
| 102 | 0.0652 | 33 | 20 | 305 |
| 103 | 172 | 4440 | >10000 | >100 |
| 104 | 1.87 | 897 | 1560 | 834 |
| 105 | 0.457 | 312 | 470 | 1028 |
| 106 | >10000 | >10000 | >10000 | — |
| 107 | 528 | >10000 | >10000 | >100 |
| 108 | 1.77 | 1730 | 1650 | 932 |
| 109 | 30.1 | 4180 | 8400 | 279 |
| 110 | 1.89 | 66 | 167 | 88 |
| 111 | 0.18 | 92 | 107 | 594 |
| 112 | 4.07 | 48 | 100 | 24 |
| 113 | 10.4 | 280 | 1710 | 164 |
| 114 | 93.3 | 2110 | 4100 | 44 |
| 115 | 25.7 | 6450 | 6320 | 246 |
| 116 | 11.0 | 3790 | 4470 | 406 |
| 117 | 46.2 | 5960 | >10000 | >100 |
| 118 | 34.0 | 9190 | 9920 | 292 |
| 119 | 49.0 | 6530 | 8590 | 175 |
| 120 | 7.38 | 5220 | 7120 | 965 |
| 121 | 210 | >10000 | >10000 | >100 |
| 122 | >10000 | >10000 | >10000 | — |
| 123 | >10000 | >10000 | >10000 | — |
| 124 | 0.828 | 3350 | 843 | 1018 |
| 125 | 89.3 | >10000 | >10000 | >100 |
| 126 | 38.4 | 5450 | 4430 | 115 |
| 127 | 2.05 | 1550 | 698 | 340 |
| 128 | 1.29 | 1120 | 500 | 388 |
| 129 | 85.9 | >10000 | >10000 | >100 |
| 130 | 1.28 | 3580 | 1330 | 1039 |
| 131 | 0.201 | 152 | 106 | 527 |
| 132 | 0.889 | 1280 | 806 | 907 |
| 133 | 0.785 | 111 | 259 | 330 |
| 134 | 1.38 | 224 | 348 | 252 |
| 135 | 8.87 | 226 | 475 | 54 |
| 136 | 129 | — | 7930 | 61 |
| 137 | >10000 | — | >10000 | — |
| 138 | >10000 | >10000 | >10000 | — |
| 139 | 46.4 | — | 5760 | 124 |
| 140 | 2.65 | 1770 | 855 | 323 |
| 141 | 30.0 | — | 3050 | 102 |
| 142 | 21.2 | — | 4170 | 197 |
| 143 | 7.08 | 2930 | 2230 | 315 |
| 144 | 225 | 912 | 1320 | 6 |
| 145 | 1.93 | 1040 | 1060 | 549 |
| 146 | 0.202 | 21 | 47 | 234 |
| 147 | 84.4 | 5530 | >10000 | >100 |
| 148 | 0.0924 | 12 | 28 | 305 |
| 149 | 739 | >10000 | >10000 | >100 |
| 150 | 35.3 | >10000 | >10000 | >100 |
| 151 | 21.7 | 8120 | 8270 | 381 |
| 152 | 3.41 | 496 | 2680 | 786 |
| 153 | 18.9 | 2000 | 3200 | 169 |
| 154 | 0.237 | 229 | 409 | 1726 |
| 155 | >10000 | >10000 | >10000 | — |
| 156 | 639 | 111 | 407 | 1 |
| 157 | >10000 | >10000 | >10000 | — |
| 158 | 7.71 | 170 | 394 | 51 |
| 159 | 1.45 | 1820 | 591 | 408 |
| 160 | 37.3 | 444 | 922 | 25 |
| 161 | 0.57 | 275 | 318 | 558 |
| 162 | 4.5 | 6050 | 6260 | 1391 |
| 163 | 4.78 | >10000 | 9450 | 1977 |
| 164 | 11.3 | 8340 | 8070 | 714 |
| 165 | >10000 | >10000 | >10000 | >100 |
| 166 | 3.12 | >10000 | >10000 | >100 |
| 167 | 10.7 | >10000 | >10000 | >100 |
| 168 | 40.3 | >10000 | >10000 | >100 |
| 169 | 3.72 | 1820 | 1750 | 470 |
| 170 | >10000 | >10000 | >10000 | — |
| 171 | 559 | 7790 | >10000 | >100 |
| 172 | 436 | >10000 | >10000 | >100 |
| 173 | 0.104 | 6 | 9 | 83 |
| 174 | 32.7 | >10000 | >10000 | >100 |
| 175 | 0.189 | 201 | 55 | 289 |
| 176 | 1.49 | 146 | 149 | 100 |
| 177 | 3.91 | 43 | 99 | 25 |
| 178 | 0.598 | 1180 | 722 | 1207 |
| 179 | 0.0959 | 305 | 220 | 2294 |
| 180 | 23.8 | >10000 | 7180 | 302 |
| 181 | 6.04 | 761 | 1000 | 166 |
| 182 | 14.5 | 1320 | 2610 | 180 |
| 184 | 0.0776 | 30.7 | 33.4 | 430 |
| 185 | 7.43 | 1560 | 2240 | 301 |
| 188 | 0.267 | 1520 | 802 | 3004 |
| 191 | 0.329 | 310 | 340 | 1033 |
| 192 | 28.6 | 3130 | 3010 | 105 |
| 193 | 0.355 | 1040 | 381 | 1073 |
| 194 | — | 4860 | — | — |
| 196 | 1.96 | 3430 | 1600 | 816 |
| 198 | 137 | 3180 | — | — |
| 200 | 4.28 | 1400 | 1470 | 343 |
| 201 | 10.2 | 6700 | 8340 | 818 |
| 202 | 31.0 | — | — | — |
| 203 | >1000 | >10000 | >10000 | >10000 |
| 204 | >1000 | >10000 | >10000 | >10000 |
| 207 | 0.375 | 1220 | 815 | 2173 |
| 208 | 0.658 | 2090 | 1710 | 2599 |
| 209 | 1.26 | 2200 | 905 | 718 |
| 217 | 6.45 | 6810 | — | — |
| 218 | 19.1 | 6600 | — | — |
| 219 | 0.186 | 582 | 781 | 4199 |
| 220 | 1.49 | 5700 | 8960 | 6013 |
| 224 | 0.0926 | 249 | 289 | 3121 |
| 226 | 1.48 | 2110 | 1370 | 926 |
| 227 | 0.458 | 704 | 205 | 448 |
| 229 | 0.383 | 531 | 298 | 778 |
| 230 | 0.412 | 856 | 472 | 1146 |
| 238 | 4.24 | 2670 | 4610 | 1087 |
| 239 | 10.1 | 3780 | 3880 | 384 |
| 240 | 5.5 | 387 | 5030 | 915 |
| 241 | 4.73 | 5010 | 5560 | 1175 |
| 243 | 4.39 | 4450 | 5880 | 1339 |
| 244 | 1.63 | 5870 | 9550 | 5859 |
| 245 | 145 | — | — | — |
| 250 | 11.7 | 8370 | 6920 | 591 |
| 251 | 64.6 | — | — | — |
| 252 | 0.511 | 529 | 386 | 755 |
| 254 | 40.8 | 9080 | — | — |
| 255 | 1.26 | 1360 | 1630 | 1294 |
| 256 | 8.35 | 5440 | 3590 | 430 |
| 257 | 10.1 | 3960 | 5070 | 502 |
| 262 | — | — | — | — |
| 263 | 11.6 | 440 | 1070 | 092 |
| 264 | — | — | — | — |
| 265 | 200 | 875 | 2780 | 014 |
| 266 | — | 5040 | — | — |
| 267 | 164 | 553 | 901 | 005 |
| 268 | — | 3020 | 4470 | — |
| 269 | 26.3 | — | — | — |
| 270 | 0.792 | 312 | 613 | 774 |
| 271 | 111 | — | 5870 | 053 |
| 272 | 0.0732 | 39.2 | 18.9 | 258 |
| 273 | 3.8 | | 1090 | 287 |
| 274 | 147 | 8430 | — | — |
| 275 | 0.559 | 158 | 338 | 605 |
| 278 | 93.5 | 7860 | — | — |
| 279 | 0.0922 | 8.95 | 14.9 | 162 |
| 280 | 8.84 | 1720 | 2150 | 243 |
| 281 | 0.0568 | 2.98 | 4.92 | 87 |
| 282 | 10.4 | 6080 | 5240 | 504 |
| 283 | 10.9 | 2970 | 7160 | — |
| 284 | 3.19 | 2920 | 2250 | — |
| 285 | — | — | — | — |
| 286 | 482 | 1510 | 1830 | — |
| 287 | — | — | — | — |
| 288 | — | — | 9680 | — |
| 289 | — | — | — | — |
| 290 | 81.4 | — | — | — |
| 291 | 41.9 | — | — | — |
| 292 | — | — | — | — |
| 293 | 1.76 | 46.1 | 59.4 | — |
| 294 | 1.45 | 90.0 | 118 | — |
| 295 | 0.171 | 107 | 45.5 | — |
| 296 | 8.97 | 4690 | 1650 | — |
| 297 | 323 | — | — | — |
| 298 | 1.51 | 8030 | 3280 | — |
| 299 | 175 | — | — | — |
| 300 | — | — | — | — |
| 301 | — | — | — | — |
| 302 | 857 | 346 | 4370 | — |
| 303 | 1.05 | 119 | 239 | — |
| 304 | 0.908 | 4.59 | 15.9 | — |
| 305 | 677 | 9580 | — | — |
| 306 | 531 | — | — | — |
| 307 | 3.33 | 1420 | 3210 | — |
| 308 | 0.0594 | 35.6 | 47.0 | — |
| 309 | 0.434 | 1010 | 1070 | — |
| 310 | 106 | 471 | 3040 | — |
| 311 | 341 | 242 | 1450 | — |
| 312 | — | — | — | — |
| 313 | 8.74 | 5230 | — | — |
| 315 | 3.79 | 2580 | 2250 | — |
| 316 | — | 8660 | — | — |
| 317 | — | 6550 | — | — |
| 318 | 26.4 | 3030 | 8830 | — |
| 320 | 0.0637 | 86.7 | 58.5 | — |
| 322 | 156 | — | — | — |
| 323 | 7.4 | 445 | 734 | — |
| 325 | 0.163 | 87.1 | 31.3 | — |
| 328 | 509 | — | — | — |
| 329 | 307 | 5900 | 5720 | — |
| 330 | 0.626 | 1230 | 1320 | — |
| 331 | 0.0353 | 33.2 | 30.0 | — |
| 333 | 1.26 | 4470 | 4360 | — |
| 334 | 1.43 | 1920 | 4170 | — |
| 335 | 0.0513 | 61.3 | 57.7 | — |
| 337 | 2.35 | 5200 | 8030 | — |
| 338 | 0.0678 | 28.3 | 22.7 | — |
| 341 | 901 | 2030 | — | — |
| 342 | 3.77 | 28.8 | 210 | — |
| 343 | — | 1720 | — | — |
| 344 | — | 5670 | — | — |
| 345 | 30.6 | 55.7 | 253 | — |
| 346 | — | — | — | — |
| 347 | 0.0423 | 15.8 | 9.02 | — |
| 349 | 3.99 | 1330 | 1480 | — |
| 350 | 719 | 7000 | — | — |
| 351 | 4.43 | 95.7 | 340 | — |
| 352 | 108 | 1530 | — | — |
| 353 | 0.244 | 35.0 | 67.8 | — |
| 355 | 0.315 | 41.6 | 149 | — |
| 356 | 11.8 | 2050 | 5380 | — |
| 357 | 31.0 | 1770 | 4880 | — |
| 359 | 0.402 | 58.8 | 68.5 | — |
| 360 | 14.3 | 3430 | 3210 | — |
| 362 | 0.727 | 19.0 | 73.3 | — |
| 363 | 48.3 | 5500 | — | — |
| 364 | 54.7 | 5770 | 9390 | — |
| 365 | 46.2 | 3040 | 6160 | — |
| 366 | 21.9 | 1320 | 7070 | — |
| 367 | 0.058 | 26.5 | 47.4 | — |
| 369 | 4.66 | 1140 | 1480 | — |
| 370 | 6.98 | 1740 | 3880 | — |
| 371 | 2.83 | 3730 | 2740 | — |
| 372 | 0.0597 | 36.0 | 30.5 | — |
| 373 | 0.0597 | 36.0 | 30.5 | — |
| 374 | 1.23 | 128 | 146 | — |
| 375 | 22.1 | 98.3 | 807 | — |
| 376 | 7.99 | 115 | 344 | — |
| 377 | 40.6 | 133 | 671 | — |
| 378 | 6.54 | 69.2 | 278 | — |
| 379 | 64.1 | 141 | 741 | — |
| 380 | 336 | 101 | 662 | — |
| 381 | 183 | — | — | — |
| 382 | 1.81 | 435 | 433 | — |
| 384 | 0.664 | 2040 | 3040 | — |
| 385 | — | 9780 | — | — |
| 386 | 10.5 | 6920 | — | — |
| 387 | 27.3 | 7210 | — | — |
| 389 | 4.05 | 502 | 914 | — |
| 390 | 38.1 | 162 | 713 | — |
| 391 | 0.237 | 1350 | 776 | — |
| 392 | 5.64 | — | — | — |
| 393 | 98.6 | 576 | 1610 | — |
| 394 | 63.3 | 661 | 1890 | — |
| 395 | 1.19 | 30.3 | 52.6 | — |
| 396 | 0.488 | 1190 | 235 | — |
| 397 | 382 | 3360 | 6060 | — |
| 398 | 625 | 5730 | — | — |
| 399 | — | 4320 | 9270 | — |
| 400 | — | 5200 | — | — |
| 401 | 33.8 | 885 | 1280 | — |
| 402 | 0.222 | 16.9 | 6.48 | — |
| 403 | 17.3 | 1810 | 1400 | — |
| 404 | 18.0 | 498 | 685 | — |
| 405 | 31.0 | 3300 | 2380 | — |
| 406 | 92.9 | 5320 | 4740 | — |
| 408 | 0.413 | 874 | 110 | — |
| 409 | 0.546 | 863 | 177 | — |
| 410 | 1.08 | 2020 | 542 | — |
| 411 | 9.53 | 9390 | 2270 | — |
| 414 | 59.2 | 3250 | 1040 | — |
| 415 | 28.1 | 3000 | 2150 | — |
| 418 | 50.6 | 3820 | 3080 | — |
| 419 | 1.87 | 4990 | 1920 | — |
| 420 | 0.379 | 1240 | 291 | — |
| 421 | 0.915 | 3130 | 1190 | — |
| 423 | 50.5 | — | 3900 | — |
| 424 | 28.8 | 6840 | 5440 | — |

Compounds 3, 4, 6, 7, 9, 18, 24, 25, 28, 34, 40, 43, 46, 48, 49, 50, 53, 57, 58, 59, 65, 66, 67, 68, 79, 80, 81, 83, 84, 85, 86, 87, 90, 98, 99, 101, 102, 104, 105, 108, 110, 111, 112, 120, 124, 127, 128, 130, 131, 132, 33, 134, 135, 140, 143, 145, 146, 148, 152, 154, 158, 159, 161, 162, 163, 166, 169, 173, 175, 176, 177, 178, and 179 showed IC₅₀ values of less than 10 nM against Btk and several compounds showed >100 fold selectivity towards Btk over TEC.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound of Formula (I) having the structure: 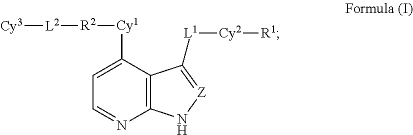
or a pharmaceutically acceptable salt thereof; or a stereoisomer or an isotopic variant thereof;
wherein:
Z is C(R⁹), or N;
R⁹ is H, halo, substituted or unsubstituted C₁-C₆alkyl, OR^{13a}, —NR^{13a}R^{13b}, —SR^{13a}, C₁-C₄alkoxyC₁-C₄alkyl, hydroxyC₁-C₄alkyl, haloC₁-C₄alkyl, haloC₁-C₄alkoxy, cyano, substituted or unsubstituted C₃-C₈cycloalkyl, substituted or unsubstituted C₂-C₇heterocycloalkyl, substituted or unsubstituted aryl, or substituted or unsubstituted heteroaryl;
each R^{13a} and R^{13b} is independently H, substituted or unsubstituted C₁-C₆alkyl, or substituted or unsubstituted C₃-C₈cycloalkyl;
Cy¹ is substituted or unsubstituted aryl or substituted or unsubstituted heteroaryl;
Cy² is substituted or unsubstituted C₃-C₈cycloalkyl, substituted or unsubstituted C₂-C₇heterocycloalkyl, substituted or unsubstituted C₂-C₇heterocycloalkenyl, or substituted or unsubstituted C₃-C₈cycloalkenyl;
Cy³ is substituted or unsubstituted C₃-C₈cycloalkyl, substituted or unsubstituted C₂-C₇heterocycloalkyl, substituted or unsubstituted C₂-C₇heterocycloalkenyl, substituted or unsubstituted C₃-C₈cycloalkenyl, substituted or unsubstituted aryl, or substituted or unsubstituted heteroaryl;
L¹ is a single bond, substituted or unsubstituted C₁-C₄alkylene, —N(R⁵)—, —O—, or —S—;
R⁵ is H, substituted or unsubstituted C₁-C₆alkyl, substituted or unsubstituted C₃-C₈cycloalkyl, or —C(O)—R^{5a};
R^{5a} is substituted or unsubstituted C₁-C₄alkyl, substituted or unsubstituted C₃-C₈cycloalkyl, substituted or unsubstituted C₂-C₇heterocycloalkyl, substituted or unsubstituted aryl, or substituted or unsubstituted heteroaryl;
L² is —N(R^{10a})C(O)—, —C(O)N(R^{10a})—, —N(R^{10a})C(O)N(R^{10b}), —O—, —S—, —S(O)—, —S(O)ₚ—, —N(R^{10a})S(O)ₚ—, or —S(O)ₚN(R^{10a})—; or L² and Cy³, taken together with the atoms to which they are attached, form a 9-14 membered bicyclic or tricyclic heterocyclyl which is unsubstituted or substituted with one or more substituents selected from C₁-C₆ alkyl, C₃-C₆ cycloalkyl, hydroxy, and carbonyl;
each R^{10a} and R^{10b} is independently H, substituted or unsubstituted C₁-C₆alkyl, or substituted or unsubstituted C₃-C₈cycloalkyl;
p is 1 or 2;
R¹ is H, halo, substituted or unsubstituted C₁-C₆alkyl, —OR^{12a}, —NR^{12a}R^{12b}, —SR^{12a}, —C(O)—O—R^{12a}, —C(O)—C(O)—N(R^{12a})R^{12b}, —C(O)—N(R^{12a})R^{12b}, —S(O)ₚ—N(R^{12a})R^{12b}, C₁-C₄alkoxyC₁-C₄alkyl, hydroxyC₁-C₄alkyl, haloC₁-C₄alkyl, haloC₁-C₄alkoxy, cyano, substituted or unsubstituted C₃-C₈cycloalkyl, substituted or unsubstituted C₂-C₇heterocycloalkyl, substituted or unsubstituted aryl, or substituted or unsubstituted heteroaryl;
each R^{12a} and R^{12b} is independently H, substituted or unsubstituted C₁-C₆alkyl, or substituted or unsubstituted C₃-C₈cycloalkyl; or
R¹ is —C(O)—R^{1a}, —C(S)—R^{1a}, —S(O)_{q}—R^{1a}; —N(R^{12a})—C(O)R^{1a}, or —N(R^{12a})—S(O)_{q}R^{1a};
R^{1a} is H, substituted or unsubstituted C₁-C₄alkyl, substituted or unsubstituted C₂-C₄alkenyl, substituted or unsubstituted C₂-C₄alkynyl, substituted or unsubstituted C₃-C₈cycloalkyl, substituted or unsubstituted C₂-C₇heterocycloalkyl, substituted or unsubstituted aryl, or substituted or unsubstituted heteroaryl;
q is 1 or 2; and,
R² is a single bond, substituted or unsubstituted C₁-C₄alkylene, or substituted or unsubstituted C₃-C₆cycloalkylene.

2. The compound according to claim 1, wherein —R²-L²-Cy³ is —R²—N(R^{10a})C(O)-Cy³, —R²—C(O)N(R^{10a})-Cy³, or —R²—O-Cy³.

3. The compound according to any one of the preceding claims, wherein R^{10a} is H, Me, Et, i-Pr, or n-Pr.

4. The compound according to any one of the preceding claims, wherein —R²-L²-Cy³ is —R²—N(H)C(O)-Cy³.

5. The compound of claim 1, wherein L²-Cy³ is: 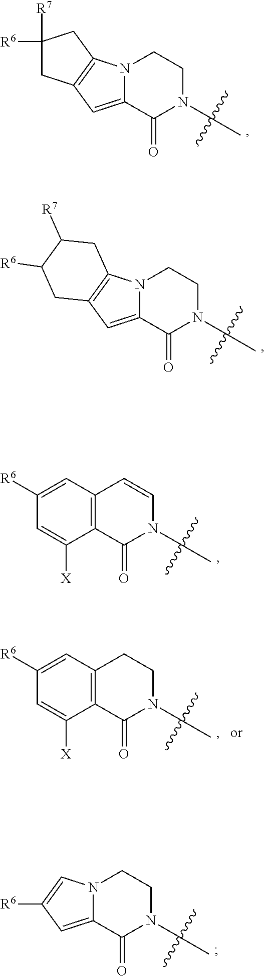
and
wherein:
each instance of R¹⁴ is independently selected from the group consisting of H, C₁₋₆ alkyl, and C₃₋₆ cycloalkyl; and
X is halo.

6. The compound of claim 5, wherein L²-Cy³ is:

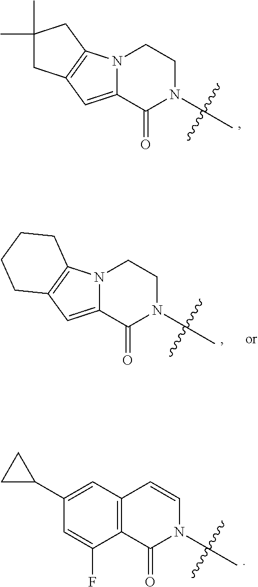 [SMILES: *N1CCn2c(cc3c2CC(C)(C)C3)C1=O ; *N1CCn2c(cc3c2CCCC3)C1=O ; *n1ccc2cc(C3CC3)cc(F)c2c1=O]

7. The compound according to any one of claims 1-6, wherein R² is unsubstituted C₁-C₄alkylene or C₁-C₄alkylene substituted with —OH, halo, or C₁-C₄alkyl.

8. The compound according to any one of claims 1-7, wherein R² is —CH₂—, —C(H)Me-, —C(Me)₂-, or
cyclopropyl.

9. The compound according to any one of claims 1-8 wherein the compound is of Formula (II) having the structure: 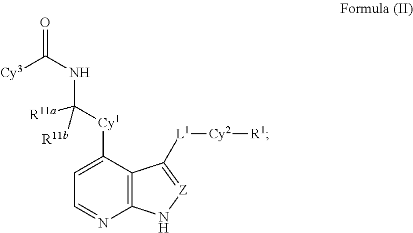
or a pharmaceutically acceptable salt thereof; or a stereoisomer or an isotopic variant thereof;
wherein
each R^{11a} and R^{11b} is independently H or substituted or unsubstituted C₁-C₄alkyl; or
R^{11a} and R^{11b} may join together with the carbon atom to which they are attached to form a substituted or unsubstituted C₃-C₆cycloalkylene; and
wherein the substitutions on R^{11a} and R^{11b}, if present, are independently selected from —OH, halo, or C₁-C₄alkyl.

10. The compound according to any one of claims 1-9, wherein Cy¹ is substituted or unsubstituted phenyl, substituted or unsubstituted pyridyl, or substituted or unsubstituted pyrimidinyl.

11. The compound according to any one of claims 1-10, wherein Cy¹ is substituted or unsubstituted phenyl.

12. The compound according to claim 11, wherein Cy¹ is 3-fluorophenyl.

13. The compound according any one of claims 9-12, wherein the compound is of Formula (III) having the structure: 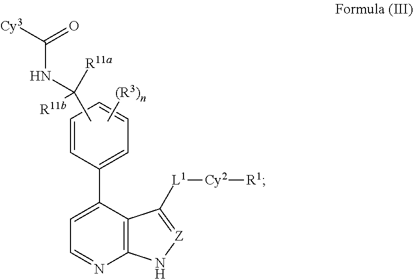
or a pharmaceutically acceptable salt thereof; or a stereoisomer or an isotopic variant thereof;
wherein
each R³ is each independently halo, substituted or unsubstituted C₁-C₆alkyl, —OR^{21a}, —NR^{21a}R^{21b}, —SR^{21a}, —C(O)—O—R^{21a}, —C(O)—C(O)—N(R^{21a})R^{21b}, —C(O)—N(R^{21a})R^{21b}, —N(R^{21a})C(O)—R^{21b}, —S(O)ₜ—N(R^{21a})R^{21b}, C₁-C₄alkoxyC₁-C₄alkyl, hydroxyC₁-C₄alkyl, haloC₁-C₄alkyl, haloC₁-C₄alkoxy, cyano, substituted or unsubstituted C₃-C₈cycloalkyl, substituted or unsubstituted C₂-C₇heterocycloalkyl, substituted or unsubstituted aryl, substituted or unsubstituted heteroaryl;
each R^{21a} and R^{21b} is independently H, substituted or unsubstituted C₁-C₆alkyl, or substituted or unsubstituted C₃-C₈cycloalkyl; and
n is 0-4.

14. The compound according to claim 13, wherein the compound is of Formula (IIIa) having the structure:

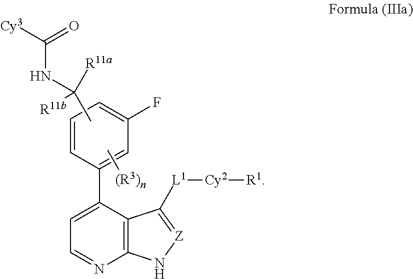

15. The compound according to any one of claims 1-14, wherein L¹ is a single bond, —N(R⁵)—, or —O—.

16. The compound according to claim 15, wherein L¹ is a single bond.

17. The compound according to claim 15, wherein L¹ is —N(R⁵)— or —O—.

18. The compound according to claim 15, wherein L¹ is —N(R⁵)—.

19. The compound according to any one of claims 1-18, wherein R⁵ is H or Me.

20. The compound according to any one of claims 1-18, wherein R⁵ is H.

21. The compound according to any one of claims 13-20, wherein the compound is of Formula (IVa) or (IVb) having the structure: 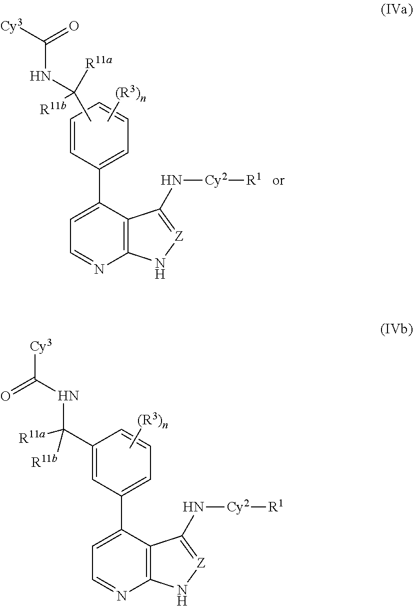
or a pharmaceutically acceptable salt thereof; or a stereoisomer or an isotopic variant thereof.

22. The compound according to any one of claims 1-21, wherein Cy² is substituted or unsubstituted C₃-C₈cycloalkyl, or substituted or unsubstituted C₃-C₈cycloalkenyl.

23. The compound according to claim 22, wherein Cy² is substituted or unsubstituted C₃-C₇cycloalkyl.

24. The compound according to claim 23, wherein Cy² is cyclopropyl, cyclobutyl, cyclopentyl, cyclohexyl, or cycloheptyl.

25. The compound according to any one of claims 1-21, wherein Cy² is substituted or unsubstituted C₂-C₇heterocycloalkyl, or substituted or unsubstituted C₂-C₇heterocycloalkenyl.

26. The compound according to claim 25, wherein Cy² is substituted or unsubstituted C₂-C₇heterocycloalkyl.

27. The compound according to claim 26, wherein Cy² is substituted or unsubstituted pyrrolidinyl, substituted or unsubstituted piperidinyl, substituted or unsubstituted morpholinyl, or substituted or unsubstituted piperazinyl, oxanyl, 1,1-dioxo-1λ⁶-thiomorpholinyl, 2-oxo-pyrrolidinyl, pyrrolidin-3-ylidene, 2,3-dioxopiperazinyl, or 1,1-dioxo-1λ⁶-thianyl.

28. The compound according to claim 26, wherein Cy² is substituted or unsubstituted pyrrolidinyl, substituted or unsubstituted piperidinyl, substituted or unsubstituted morpholinyl, or substituted or unsubstituted piperizinyl.

29. The compound according to claim 28, wherein Cy² is substituted or unsubstituted pyrrolidinyl or substituted or unsubstituted piperidinyl.

30. The compound according to claim 25, wherein Cy² is substituted or unsubstituted dihydropyrrolyl or substituted or unsubstituted tetrahydropyridyl.

31. The compound according to any one of claims 1-30, wherein Z is C(R⁹).

32. The compound according to any one of claims 1-31, wherein Z is C(R⁹); and R⁹ is H or substituted or unsubstituted C₁-C₆alkyl.

33. The compound according to claim 32, wherein Z is C(R⁹); and R⁹ is H, F, Me, Et, n-Pr, i-Pr, n-Bu, i-Bu, sec-Bu, t-Bu, cyclopropyl, or CF₃.

34. The compound according to claim 33, wherein Z is CH.

35. The compound according to any one of claims 1-30, wherein Z is C(R⁹); and R⁹ is F or CF₃.

36. The compound according to any one of claims 1-30, wherein Z is N.

37. The compound according to any one of claims 21-36, wherein the compound is of Formula (Va), (Vb), (Vc), or (Vd) having the structure: 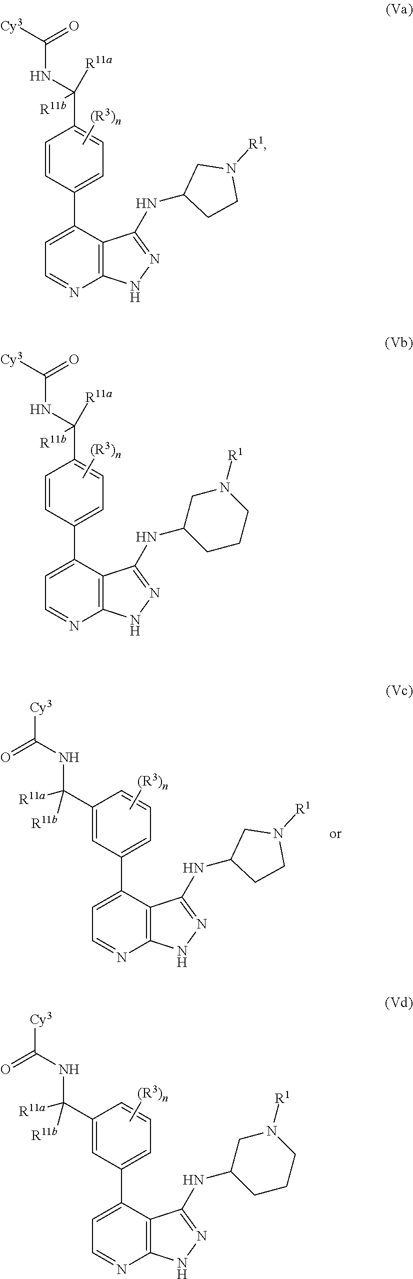
or a pharmaceutically acceptable salt thereof; or a stereoisomer or an isotopic variant thereof.

38. The compound according to any one of the preceding claims, wherein R¹ is H, CN, —C(O)—R^{1a}, —C(O)—N(R^{12a})R^{12b}, N(R^{12a})R^{12b}, —N(R^{12a})—C(O)R^{1a}, —C(S)—R^{1a}, —S(O)ₚ—R^{1a}, or —S(O)ₚ—N(R^{12a})R^{12b}.

39. The compound according to any one of the preceding claims, wherein R¹ is H, CN, —C(O)—R^{1a}, —C(S)—R^{1a}, —S(O)ₚ—R^{1a}, or —S(O)ₚ—N(R^{2a})R^{12b}.

40. The compound of any one of the preceding claims, wherein R¹ is —C(O)—N(R^{12a})R^{12b}, N(R^{12a})R^{12b}, or —N(R^{12a})—C(O)R^{1a}.

41. The compound according to any one of the preceding claims, wherein R¹ is —C(O)—R^{1a}.

42. The compound according to any one of the preceding claims, wherein R^{1a} is substituted or unsubstituted C₁-C₄alkyl.

43. The compound according to any one of the preceding claims, wherein R^{1a} is substituted or unsubstituted C₂-C₄alkenyl.

44. The compound according to any one of the preceding claims, wherein R^{1a} is substituted with CN, substituted or unsubstituted C₁-C₆alkyl, haloC₁-C₆alkyl, substituted or unsubstituted C₃-C₈cycloalkyl, substituted or unsubstituted aryl, substituted or unsubstituted heteroaryl, hydroxyl, substituted or unsubstituted hydroxyC₁-C₄alkyl, substituted or unsubstituted aminoC₁-C₄alkyl, or substituted or unsubstituted C₁-C₄alkoxyC₁-C₄alkyl.

45. The compound according to claim any one of the preceding claims, wherein R^{1a} is substituted or unsubstituted ethenyl.

46. The compound according to any one of the preceding claims, wherein R^{1a} is ethenyl and is unsubstituted or substituted with aminoC₁-C₄alkyl.

47. The compound according to claim 46, wherein R^{1a} is ethenyl and is substituted with C₁-C₄alkylaminoC₁-C₄alkyl, C₃-C₈cycloalkylaminoC₁-C₄alkyl, or diC₁-C₄alkylaminoC₁-C₄alkyl.

48. The compound according to any one of the preceding claims, wherein R^{1a} is selected from H, CN, 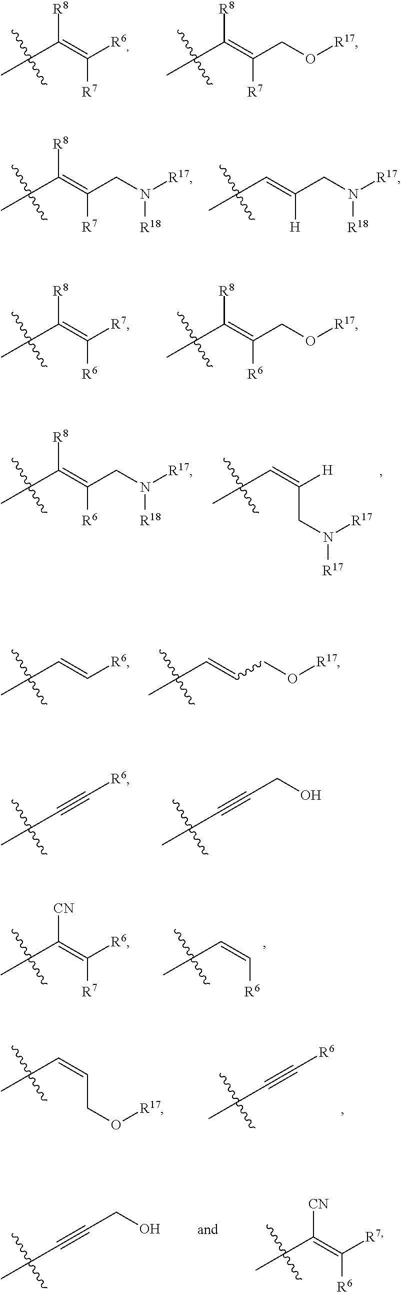
wherein R⁶, R⁷ and R⁸ are each independently H, CN, halo, substituted or unsubstituted C₁-C₄alkyl, substituted or unsubstituted C₃-C₈cycloalkyl, substituted or unsubstituted C₂-C₇heterocycloalkyl, substituted or unsubstituted C₆-C₁₂aryl, or substituted or unsubstituted 5- to 8-membered heteroaryl; or R⁷ and R⁸ together form a bond; and
R¹⁷ and R¹⁸ are independently H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted 3- to 8-membered heteroaryl.

49. The compound according to claim 48, wherein the compound is of Formula (VIa), (VIb), (VIc), or (VId) having the structure: 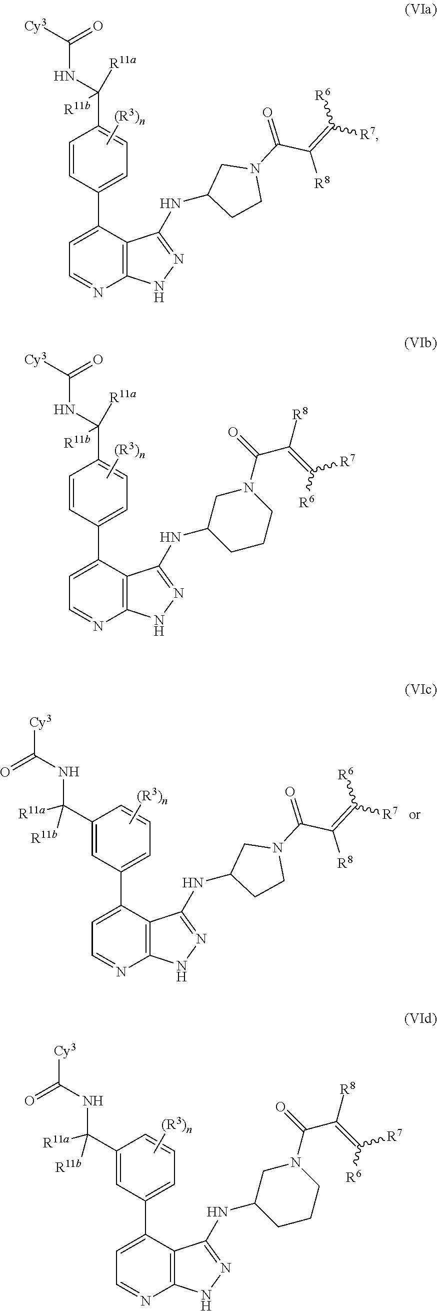
or a pharmaceutically acceptable salt thereof; or a stereoisomer or an isotopic variant thereof.

50. The compound according to any one of claims 13-49, wherein n is 0.

51. The compound according to any one of claims 13-49, wherein n is 1 or 2, and each R³ is independently halo, CN, C₁-C₄alkyl, haloC₁-C₄alkyl, C₃-C₈cycloalkyl, hydroxyl, or C₁-C₄alkoxy.

52. The compound according to claim 51, wherein n is 1 or 2, and each R³ is independently Cl, F, CN, Me, Et, t-Bu, CHF₂, CF₃, cyclopropyl, hydroxyl, or methoxy.

53. The compound according to claim 52, wherein n is 1, and R³ is F.

54. The compound according to claim 53, wherein the compound is of Formula (VIIa), (VIIb), (VIIc), or (VIId) having the structure: 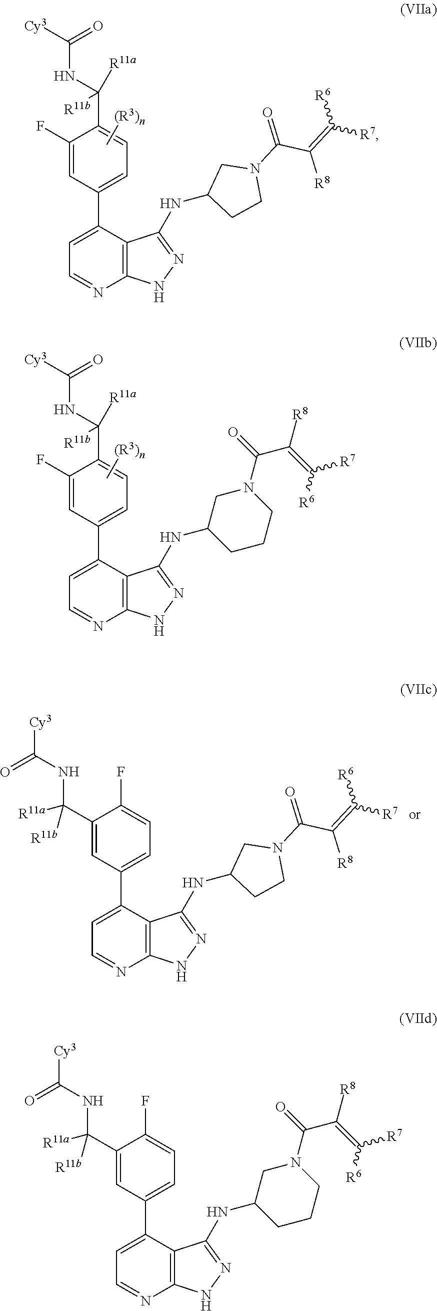
or a pharmaceutically acceptable salt thereof; or a stereoisomer or an isotopic variant thereof.

55. The compound according to any one of the preceding claims, wherein Cy³ is substituted or unsubstituted C₃-C₈cycloalkyl, or a substituted or unsubstituted C₂-C₇heterocycloalkyl.

56. The compound according to any one of the preceding claims, wherein Cy³ is substituted or unsubstituted phenyl.

57. The compound according to claim 56, wherein Cy³ is phenyl substituted with one or more of halo, CN, C₁-C₄alkyl, C₁-C₄haloalkyl, C₃-C₈cycloalkyl, C₁-C₄hydroxyalkyl, hydroxyl, or C₁-C₄alkoxy.

58. The compound according to claim 57, wherein Cy³ is phenyl substituted with one or more of Me, Et, i-Pr, n-Pr, t-Bu, —C(Me)₂-OH, F, Cl, Br, —OMe, CF₃, CN, or cyclopropyl.

59. The compound according to claim 58, wherein Cy³ is phenyl substituted with i-Pr, t-Bu, or cyclopropyl.

60. The compound according to any one of the preceding claims, wherein Cy³ is substituted or unsubstituted heteroaryl.

61. The compound according to claim 60, wherein Cy³ is substituted or unsubstituted furanyl, substituted or unsubstituted pyrrolyl, substituted or unsubstituted thienyl, substituted or unsubstituted imidazolyl, substituted or unsubstituted pyrazolyl, substituted or unsubstituted oxazolyl, substituted or unsubstituted isoxazolyl, substituted or unsubstituted thiazolyl, substituted or unsubstituted oxadiazolyl, substituted or unsubstituted thiadiazolyl, substituted or unsubstituted pyridyl, substituted or unsubstituted pyrimidinyl, or substituted or unsubstituted 4,5,6,7-tetrahydro-1,3-benzothiazole.

62. The compound according to any one of claims 60-61, wherein Cy³ is substituted or unsubstituted furanyl, substituted or unsubstituted pyrrolyl, substituted or unsubstituted thienyl, substituted or unsubstituted imidazolyl, substituted or unsubstituted pyrazolyl, substituted or unsubstituted oxazolyl, substituted or unsubstituted isoxazolyl, substituted or unsubstituted thiazolyl, substituted or unsubstituted oxadiazolyl, substituted or unsubstituted thiadiazolyl, substituted or unsubstituted pyridyl, or substituted or unsubstituted pyrimidinyl.

63. The compound according to claim 62, wherein Cy³ is unsubstituted or substituted with one or more of halo, CN, C₁-C₄alkyl, haloalkyl, C₃-C₈cycloalkyl, hydroxyl, hydroxyalkyl, or alkoxy.

64. The compound according to claim 63, wherein Cy³ is unsubstituted or substituted with one or more of halo, CN, C₁-C₄alkyl, haloalkyl, C₃-C₈cycloalkyl, hydroxyl, or alkoxy.

65. The compound according to claim 63 or 64, wherein Cy³ is oxazolyl, thiazolyl, oxadiazolyl, or thiadiazolyl.

66. The compound according to claim 65, wherein Cy³ is oxadiazolyl.

67. The compound according to claim 66, wherein Cy³ is unsubstituted or substituted with one or more of Cl, F, Me, t-Bu, 1-hydroxy-1-methyl-ethyl, or cyclopropyl.

68. The compound according to claim 66, wherein Cy³ is unsubstituted or substituted with one or more of Cl, F, Me, t-Bu, or cyclopropyl.

69. The compound according to claim 62, wherein Cy³ is pyridyl.

70. The compound according to claim 62, wherein Cy³ is oxazolyl, thiazolyl, oxadiazolyl, thiadiazolyl, phenyl, or pyridyl, and is substituted with one or more of Cl, F, CN, Me, Et, i-Pr, t-Bu, CHF₂, CF₃, cyclopropyl, hydroxyl, or methoxy.

71. The compound according to claim 62, wherein Cy³ is oxadiazolyl, and is unsubstituted or substituted with i-Pr, t-Bu, or cyclopropyl.

72. The compound according to any one of claims 47-71, wherein R⁸ is H, F, Cl, CN, C₁-C₃alkyl, or C₃-C₆cycloalkyl.

73. The compound according to claim 72, wherein R⁸ is H, CN, Me, or cyclopropyl.

74. The compound according to any one of claims 47-71, wherein each of R⁶, R⁷ and R⁸ is H.

75. The compound according to any one of claims 47-71, wherein R⁷ and R⁸ form a bond such that R^{1a} is ethynyl.

76. The compound according to any one of claims 47-71, wherein each of R⁷ and R⁸ is H; and R⁶ is unsubstituted C₁-C₃alkyl or substituted C₁-C₃alkyl.

77. The compound according to any one of claims 47-71, wherein R⁶ is C₁-C₃alkyl substituted with C₁-C₃alkoxy or with substituted or unsubstituted amino.

78. The compound according to claim 77, wherein
R⁶ is —(CH₂)ₘ—OR^{6a} or —(CH₂)ₘ—NR^{6a}R^{6b};
m is 1, 2, 3, or 4; and
each R^{6a} and R^{6b} is independently H, C₁-C₃alkyl, haloC₁-C₃alkyl, C₁-C₃alkoxy C₁-C₃alkyl, C₃-C₈cycloalkyl, C₂-C₇heterocycloalkyl, aryl, or heteroaryl.

79. The compound according to claim 78, wherein R⁶ is —(CH₂)ₘ—NR^{6a}R^{6b}.

80. The compound according to claim 78, wherein R⁶ is —(CH₂)ₘ—OR^{6a}.

81. The compound according to any one of claims 78-80, wherein R^{6a} and R^{6b} are, each independently, H, cyclopropyl, Me, Et, or methoxyethyl.

82. The compound according to claim 78, wherein R⁶ is aryl or heteroaryl.

83. The compound according to claim 82, wherein R⁶ is imidazolyl, pyridyl, or pyrimidinyl.

84. The compound according to claim 82, wherein R⁶ is phenyl.

85. The compound according to claim 78, wherein R⁶ is C₃-C₈cycloalkyl.

86. The compound according to claim 85, wherein R⁶ is cyclopropyl, cyclobutyl, cyclopentyl, or cyclohexyl.

87. The compound according to any one of claims 9-86, wherein each R^{11a} and R^{11b} is independently H or substituted or unsubstituted C₁-C₃alkyl.

88. The compound according to claim 87, wherein each R^{11a} and R^{11b} is independently H, Me, —CH₂OH, or Et.

89. The compound according to any one of claims 9-88, wherein each R^{11a} and R^{11b} is H.

90. The compound according to any one of claims 9-86, wherein R^{11a} and R^{11b} may join together to form a substituted or unsubstituted C₃-C₆cycloalkyl.

91. The compound according to claim 90, wherein R^{11a} and R^{11b} may join together to form a substituted or unsubstituted cyclopropyl.

92. The compound according to claim 37, wherein
Cy³ is phenyl and is unsubstituted or substituted with i-Pr, t-Bu, or cyclopropyl;
R¹ is H;
n is 0 or 1;
R³, if present, is F; and
R^{11a} and R^{11b} are H.

93. The compound according to claim 37, wherein
Cy³ is oxadiazolyl and is unsubstituted or substituted with i-Pr, t-Bu, or cyclopropyl;
R¹ is H;
n is 0 or 1;
R³, if present, is F; and
R^{11a} and R^{11b} are H.

94. A compound selected from:
5-tert-butyl-N-{1-[4-(3-{[(3R)-pyrrolidin-3-yl]amino}-1H-pyrazolo[3,4-b]pyridin-4-yl)phenyl]cyclopropyl}-1,2,4-oxadiazole-3-carboxamide;
5-tert-butyl-N-{[2-fluoro-4-(3-{[(3R)-pyrrolidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,2,4-oxadiazole-3-carboxamide;
5-tert-butyl-N-{1-[4-(3-{[(3R)-1-(prop-2-enoyl)pyrrolidin-3-yl]amino}-1H-pyrazolo[3,4-b]pyridin-4-yl)phenyl]cyclopropyl}-1,2,4-oxadiazole-3-carboxamide;
5-tert-butyl-N-{1-[4-(3-{[(3R)-1-propanoylpyrrolidin-3-yl]amino}-1H-pyrazolo[3,4-b]pyridin-4-yl)phenyl]cyclopropyl}-1,2,4-oxadiazole-3-carboxamide;
4-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}benzamide;
N-{[4-(3-{[(3R)-1-(but-2-ynoyl)pyrrolidin-3-yl]amino}-1H-pyrazolo[3,4-b]pyridin-4-yl)-2-fluorophenyl]methyl}-4-tert-butylbenzamide;
4-tert-butyl-N-{[2-fluoro-4-(3-{[(3R)-pyrrolidin-3-yl]amino}-1H-pyrazolo[3,4-b]pyridin-4-yl)phenyl]methyl}benzamide;
4-tert-butyl-N-[2-methyl-3-(3-{[(3R)-1-(prop-2-enoyl)pyrrolidin-3-yl]amino}-1H-pyrazolo[3,4-b]pyridin-4-yl)phenyl]benzamide;
N-[3-(3-{[(3R)-1-(but-2-ynoyl)pyrrolidin-3-yl]amino}-1H-pyrazolo[3,4-b]pyridin-4-yl)-2-methylphenyl]-4-tert-butylbenzamide;
(1r,4r)-4-({4-[3-fluoro-4-(hydroxymethyl)phenyl]-1H-pyrazolo[3,4-b]pyridin-3-yl}amino)cyclohexan-1-ol;
4-tert-butyl-N-{[2-fluoro-4-(3-{[(1r,4r)-4-hydroxycyclohexyl]amino}-1H-pyrazolo[3,4-b]pyridin-4-yl)phenyl]methyl}benzamide;
4-tert-butyl-N-[3-(3-{[(1r,4r)-4-hydroxycyclohexyl]amino}-1H-pyrazolo[3,4-b]pyridin-4-yl)phenyl]benzamide;
4-tert-butyl-N-[2-methyl-3-(3-{[(1r,4r)-4-hydroxycyclohexyl]amino}-1H-pyrazolo[3,4-b]pyridin-4-yl)phenyl]benzamide;
4-tert-butyl-N-[2-fluoro-4-(3-{[(1r,4r)-4-hydroxycyclohexyl]amino}-1H-pyrazolo[3,4-b]pyridin-4-yl)phenyl]benzamide;
[2-(6-cyclopropyl-8-fluoro-1-oxo-1,2-dihydroisoquinolin-2-yl)-6-(3-{[(3R)-1-(prop-2-enoyl)pyrrolidin-3-yl]amino}-1H-pyrazolo[3,4-b]pyridin-4-yl)phenyl]methyl acetate;
6-cyclopropyl-8-fluoro-2-[2-(hydroxymethyl)-3-(3-{[(3R)-pyrrolidin-3-yl]amino}-1H-pyrazolo[3,4-b]pyridin-4-yl)phenyl]-1,2-dihydroisoquinolin-1-one;
6-cyclopropyl-8-fluoro-2-[2-(hydroxymethyl)-3-(3-{[(3R)-1-(prop-2-enoyl)pyrrolidin-3-yl]amino}-1H-pyrazolo[3,4-b]pyridin-4-yl)phenyl]-1,2-dihydroisoquinolin-1-one;
N-{[2-fluoro-4-(3-{[(1r,4r)-4-hydroxycyclohexyl]amino}-1H-pyrazolo[3,4-b]pyridin-4-yl)phenyl]methyl}-4,4-dimethylpentanamide;
1-ethyl-N-{[2-fluoro-4-(3-{[(1r,4r)-4-hydroxycyclohexyl]amino}-1H-pyrazolo[3,4-b]pyridin-4-yl)phenyl]methyl}-1H-pyrazole-4-carboxamide;
5-tert-butyl-N-{[2-fluoro-4-(3-{[(1r,4r)-4-hydroxycyclohexyl]amino}-1H-pyrazolo[3,4-b]pyridin-4-yl)phenyl]methyl}-1,2,4-oxadiazole-3-carboxamide;
4-tert-butyl-N-{1-[4-(3-{[(1r,4r)-4-hydroxycyclohexyl]amino}-1H-pyrazolo[3,4-b]pyridin-4-yl)phenyl]cyclopropyl}benzamide;
4-tert-butyl-N-{[4-(3-{[(1r,4r)-4-hydroxycyclohexyl]amino}-1H-pyrazolo[3,4-b]pyridin-4-yl)phenyl]methyl}benzamide;
N-{[4-(3-{[(3R)-1-(but-2-ynoyl)pyrrolidin-3-yl]amino}-1H-pyrazolo[3,4-b]pyridin-4-yl)-2-fluorophenyl]methyl}-5-tert-butyl-1,2,4-oxadiazole-3-carboxamide;
5-tert-butyl-N-{[4-(3-{[(3R)-1-[(2E)-4-(dimethylamino)but-2-enoyl]pyrrolidin-3-yl]amino}-1H-pyrazolo[3,4-b]pyridin-4-yl)-2-fluorophenyl]methyl}-1,2,4-oxadiazole-3-carboxamide;
N-{[2-fluoro-4-(3-{[(1r,4r)-4-hydroxycyclohexyl]amino}-1H-pyrazolo[3,4-b]pyridin-4-yl)phenyl]methyl}-4-(2-hydroxypropan-2-yl)benzamide;
5-tert-butyl-N-{[2-fluoro-4-(3-{[(1r,4r)-4-hydroxycyclohexyl]amino}-1H-pyrazolo[3,4-b]pyridin-4-yl)phenyl]methyl}-1,2-oxazole-3-carboxamide;
N-{1-[4-(3-{[(3R)-1-(but-2-ynoyl)pyrrolidin-3-yl]amino}-1H-pyrazolo[3,4-b]pyridin-4-yl)phenyl]cyclopropyl}-5-tert-butyl-1,2,4-oxadiazole-3-carboxamide;
5-tert-butyl-N-{1-[4-(3-{[(3R)-1-[(2E)-4-(dimethylamino)but-2-enoyl]pyrrolidin-3-yl]amino}-1H-pyrazolo[3,4-b]pyridin-4-yl)phenyl]cyclopropyl}-1,2,4-oxadiazole-3-carboxamide;
5-tert-butyl-N-{1-[4-(3-{[(3R)-1-(3-methyloxetane-3-carbonyl)pyrrolidin-3-yl]amino}-1H-pyrazolo[3,4-b]pyridin-4-yl)phenyl]cyclopropyl}-1,2,4-oxadiazole-3-carboxamide;
N-{[2-fluoro-4-(3-{[(3R)-pyrrolidin-3-yl]amino}-1H-pyrazolo[3,4-b]pyridin-4-yl)phenyl]methyl}benzamide;
N-{[2-fluoro-4-(3-{[(3R)-pyrrolidin-3-yl]amino}-1H-pyrazolo[3,4-b]pyridin-4-yl)phenyl]methyl}-4-methylbenzamide;
N-{[2-fluoro-4-(3-{[(3R)-1-(prop-2-enoyl)pyrrolidin-3-yl]amino}-1H-pyrazolo[3,4-b]pyridin-4-yl)phenyl]methyl}benzamide;
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-4-methylbenzamide;
4-cyclopropyl-N-{[2-fluoro-4-(3-{[(3R)-pyrrolidin-3-yl]amino}-1H-pyrazolo[3,4-b]pyridin-4-yl)phenyl]methyl}benzamide;
6-tert-butyl-2-{[2-fluoro-4-(3-{[(3R)-pyrrolidin-3-yl]amino}-1H-pyrazolo[3,4-b]pyridin-4-yl)phenyl]methyl}-1,2,3,4-tetrahydroisoquinolin-1-one;
4-tert-butyl-N-{[2-fluoro-4-(3-{[(3S)-pyrrolidin-3-yl]amino}-1H-pyrazolo[3,4-b]pyridin-4-yl)phenyl]methyl}benzamide;
6-tert-butyl-2-{[2-fluoro-4-(3-{[(1r,4r)-4-hydroxycyclohexyl]amino}-1H-pyrazolo[3,4-b]pyridin-4-yl)phenyl]methyl}-1,2,3,4-tetrahydroisoquinolin-1-one;
2-tert-butyl-N-{[2-fluoro-4-(3-{[(1r,4r)-4-hydroxycyclohexyl]amino}-1H-pyrazolo[3,4-b]pyridin-4-yl)phenyl]methyl}-1,3-oxazole-5-carboxamide;
4-cyclopropyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}benzamide;
4-cyclopropyl-N-{[2-fluoro-4-(3-{[(3R)-1-(3-methyloxetane-3-carbonyl)pyrrolidin-3-yl]amino}-1H-pyrazolo[3,4-b]pyridin-4-yl)phenyl]methyl}benzamide;
4-tert-butyl-N-[2-fluoro-4-(3-{[(3R)-pyrrolidin-3-yl]amino}-1H-pyrazolo[3,4-b]pyridin-4-yl)phenyl]benzamide;
6-tert-butyl-2-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,2,3,4-tetrahydroisoquinolin-1-one;
4-tert-butyl-N-{[2-fluoro-4-(3-{[(3S)-1-(prop-2-enoyl)pyrrolidin-3-yl]amino}-1H-pyrazolo[3,4-b]pyridin-4-yl)phenyl]methyl}benzamide;
4-tert-butyl-N-[2-fluoro-4-(3-{[(3R)-1-(prop-2-enoyl)pyrrolidin-3-yl]amino}-1H-pyrazolo[3,4-b]pyridin-4-yl)phenyl]benzamide;
3-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}benzamide;
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-4-(prop-1-en-2-yl)benzamide;
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-4-(trifluoromethyl)benzamide;
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-4-methoxybenzamide;
5-tert-butyl-N-{[2-fluoro-4-(3-{[(3S)-pyrrolidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(3S)-1-(prop-2-enoyl)pyrrolidin-3-yl]amino}-1H-pyrazolo[3,4-b]pyridin-4-yl)phenyl]methyl}-1,2,4-oxadiazole-3-carboxamide;
10-{[2-fluoro-4-(3-{[(3R)-pyrrolidin-3-yl]amino}-1H-pyrazolo[3,4-b]pyridin-4-yl)phenyl]methyl}-4,4-dimethyl-1,10-diazatricyclo[6.4.0.0²,6]dodeca-2(6),7-dien-9-one;
10-{[2-fluoro-4-(3-{[(3R)-1-(prop-2-enoyl)pyrrolidin-3-yl]amino}-1H-pyrazolo[3,4-b]pyridin-4-yl)phenyl]methyl}-4,4-dimethyl-1,10-diazatricyclo[6.4.0.0²,6]dodeca-2(6),7-dien-9-one;
5-ethyl-N-{[2-fluoro-4-(3-{[(3R)-pyrrolidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(2R,3R)-2-methylpiperidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(3R,6S)-6-methylpiperidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(2R,3R)-2-methyl-1-(prop-2-enoyl)piperidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(3R,6S)-6-methyl-1-(prop-2-enoyl)piperidin-3-yl]amino}-1H-pyrazolo[3,4-b]pyridin-4-yl)phenyl]methyl}-1,2,4-oxadiazole-3-carboxamide;
(3R)-3-({4-[4-({4,4-dimethyl-9-oxo-1,10-diazatricyclo[6.4.0.0²,6]dodeca-2(6),7-dien-10-yl}methyl)-3-fluorophenyl]-1H-pyrazolo[3,4-b]pyridin-3-yl}amino)pyrrolidine-1-carbaldehyde;
(3R)-3-[(4-{4-[(6-tert-butyl-1-oxo-1,2,3,4-tetrahydroisoquinolin-2-yl)methyl]-3-fluorophenyl}-1H-pyrazolo[3,4-b]pyridin-3-yl)amino]pyrrolidine-1-carbaldehyde;
4-tert-butyl-N-{[2-fluoro-4-(3-{[(3S)-1-formylpyrrolidin-3-yl]amino}-1H-pyrazolo[3,4-b]pyridin-4-yl)phenyl]methyl}benzamide;
3-tert-butyl-N-{[2-fluoro-4-(3-{[(3R)-pyrrolidin-3-yl]amino}-1H-pyrazolo[3,4-b]pyridin-4-yl)phenyl]methyl}benzamide;
5-tert-butyl-N-{[2-fluoro-4-(3-{[(3R)-pyrrolidin-3-yl]amino}-1H-pyrazolo[3,4-b]pyridin-4-yl)phenyl]methyl}-1,2-oxazole-3-carboxamide;
1-tert-butyl-N-{[2-fluoro-4-(3-{[(3R)-pyrrolidin-3-yl]amino}-1H-pyrazolo[3,4-b]pyridin-4-yl)phenyl]methyl}-1H-pyrazole-3-carboxamide;
5-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,2-oxazole-3-carboxamide;
1-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1H-pyrazole-3-carboxamide;
(2S,5R)-5-{[4-(4-{[(5-tert-butyl-1,2,4-oxadiazol-3-yl)formamido]methyl}-3-fluorophenyl)-1H-pyrazolo[3,4-b]pyridin-3-yl]amino}-N,N,2-trimethylpiperidine-1-carboxamide;
5-tert-butyl-N-{[2-fluoro-4-(3-{[(3R,6 S)-1-formyl-6-methylpiperidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(3R)-piperidin-3-yl]amino}-1H-pyrazolo[3,4-b]pyridin-4-yl)phenyl]methyl}-1,2,4-oxadiazole-3-carboxamide;
5-tert-butyl-N-{1-[4-(3-{[(3R)-piperidin-3-yl]amino}-1H-pyrazolo[3,4-b]pyridin-4-yl)phenyl]cyclopropyl}-1,2,4-oxadiazole-3-carboxamide;
3-tert-butyl-N-{1-[4-(3-{[(3R)-pyrrolidin-3-yl]amino}-1H-pyrazolo[3,4-b]pyridin-4-yl)phenyl]cyclopropyl}-1,2,4-oxadiazole-5-carboxamide;
5-tert-butyl-N-[(1S)-1-[4-(3-{[(3R)-pyrrolidin-3-yl]amino}-1H-pyrazolo[3,4-b]pyridin-4-yl)phenyl]ethyl]-1,2,4-oxadiazole-3-carboxamide;
5-tert-butyl-N-[(1R)-1-[4-(3-{[(3R)-pyrrolidin-3-yl]amino}-1H-pyrazolo[3,4-b]pyridin-4-yl)phenyl]ethyl]-1,2,4-oxadiazole-3-carboxamide;
5-tert-butyl-N-{[3-fluoro-5-(3-{[(3R)-pyrrolidin-3-yl]amino}-1H-pyrazolo[3,4-b]pyridin-4-yl)phenyl]methyl}-1,2,4-oxadiazole-3-carboxamide;
4-tert-butyl-N-{[3-fluoro-5-(3-{[(3R)-pyrrolidin-3-yl]amino}-1H-pyrazolo[3,4-b]pyridin-4-yl)phenyl]methyl}benzamide;
1-tert-butyl-N-{[2-fluoro-4-(3-{[(3R)-pyrrolidin-3-yl]amino}-1H-pyrazolo[3,4-b]pyridin-4-yl)phenyl]methyl}-1H-pyrazole-4-carboxamide;
5-tert-butyl-N-{[2-fluoro-4-(3-{[(3R)-pyrrolidin-3-yl]amino}-1H-pyrazolo[3,4-b]pyridin-4-yl)phenyl]methyl}-1,3,4-oxadiazole-2-carboxamide;
3-tert-butyl-N-{[2-fluoro-4-(3-{[(3R)-pyrrolidin-3-yl]amino}-1H-pyrazolo[3,4-b]pyridin-4-yl)phenyl]methyl}-1H-1,2,4-triazole-5-carboxamide;
3-tert-butyl-N-{[2-fluoro-4-(3-{[(3R)-pyrrolidin-3-yl]amino}-1H-pyrazolo[3,4-b]pyridin-4-yl)phenyl]methyl}-1,2,4-oxadiazole-5-carboxamide;
1-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1H-pyrazole-4-carboxamide;
5-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,3,4-oxadiazole-2-carboxamide;
3-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1H-1,2,4-triazole-5-carboxamide;
3-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,2,4-oxadiazole-5-carboxamide;
5-ethyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(3R)-1-(prop-2-enoyl)piperidin-3-yl]amino}-1H-pyrazolo[3,4-b]pyridin-4-yl)phenyl]methyl}-1,2,4-oxadiazole-3-carboxamide;
3-tert-butyl-N-{1-[4-(3-{[(3R)-1-(prop-2-enoyl)pyrrolidin-3-yl]amino}-1H-pyrazolo[3,4-b]pyridin-4-yl)phenyl]cyclopropyl}-1,2,4-oxadiazole-5-carboxamide;
5-tert-butyl-N-{2-[2-fluoro-4-(3-{[(3R)-1-(prop-2-enoyl)pyrrolidin-3-yl]amino}-1H-pyrazolo[3,4-b]pyridin-4-yl)phenyl]ethyl}-1,2,4-oxadiazole-3-carboxamide;
5-tert-butyl-N-{[3-fluoro-5-(3-{[(3R)-1-(prop-2-enoyl)pyrrolidin-3-yl]amino}-1H-pyrazolo[3,4-b]pyridin-4-yl)phenyl]methyl}-1,2,4-oxadiazole-3-carboxamide;
4-tert-butyl-N-{[3-fluoro-5-(3-{[(3R)-1-(prop-2-enoyl)pyrrolidin-3-yl]amino}-1H-pyrazolo[3,4-b]pyridin-4-yl)phenyl]methyl}benzamide;
5-tert-butyl-N-{[2-fluoro-4-(3-{[(3R)-1-formylpiperidin-3-yl]amino}-1H-pyrazolo[3,4-b]pyridin-4-yl)phenyl]methyl}-1,2,4-oxadiazole-3-carboxamide;
4-tert-butyl-N-[(1S)-1-[4-(3-{[(3R)-pyrrolidin-3-yl]amino}-1H-pyrazolo[3,4-b]pyridin-4-yl)phenyl]ethyl]benzamide;
N-{[4-(3-{[(3S)-1-acetylpyrrolidin-3-yl]amino}-1H-pyrazolo[3,4-b]pyridin-4-yl)-2-fluorophenyl]methyl}-5-tert-butyl-1,2,4-oxadiazole-3-carboxamide;
5-tert-butyl-N-{[2-fluoro-4-(3-{[(3S)-1-formylpyrrolidin-3-yl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(3S)-1-(3-methyloxetane-3-carbonyl)pyrrolidin-3-yl]amino}-1H-pyrazolo[3,4-b]pyridin-4-yl)phenyl]methyl}-1,2,4-oxadiazole-3-carboxamide;
5-tert-butyl-N-[(1S)-1-[4-(3-{[(3R)-1-(prop-2-enoyl)pyrrolidin-3-yl]amino}-1H-pyrazolo[3,4-b]pyridin-4-yl)phenyl]ethyl]-1,2,4-oxadiazole-3-carboxamide;
5-tert-butyl-N-[(1S)-1-[4-(3-{[(3R)-1-formylpyrrolidin-3-yl]amino}-1H-pyrazolo[3,4-b]pyridin-4-yl)phenyl]ethyl]-1,2,4-oxadiazole-3-carboxamide;
4-tert-butyl-N-[(1S)-1-[4-(3-{[(3R)-1-(prop-2-enoyl)pyrrolidin-3-yl]amino}-1H-pyrazolo[3,4-b]pyridin-4-yl)phenyl]ethyl]benzamide;
5-tert-butyl-N-[(1R)-1-[4-(3-{[(3R)-1-(prop-2-enoyl)pyrrolidin-3-yl]amino}-1H-pyrazolo[3,4-b]pyridin-4-yl)phenyl]ethyl]-1,2,4-oxadiazole-3-carboxamide;
5-tert-butyl-N-[(1R)-1-[4-(3-{[(3R)-1-formylpyrrolidin-3-yl]amino}-1H-pyrazolo[3,4-b]pyridin-4-yl)phenyl]ethyl]-1,2,4-oxadiazole-3-carboxamide;
N-[(4-{3-[(3R)-3-aminopyrrolidin-1-yl]-1H-pyrazolo[3,4-b]pyridin-4-yl}-2-fluorophenyl)methyl]-4-tert-butylbenzamide;
4-tert-butyl-N-[(2-fluoro-4-{3-[(3R)-3-(prop-2-enamido)pyrrolidin-1-yl]-1H-pyrazolo[3,4-b]pyridin-4-yl}phenyl)methyl]benzamide;
5-tert-butyl-N-[(2-fluoro-4-{3-[(3R)-3-(prop-2-enamido)pyrrolidin-1-yl]-1H-pyrazolo[3,4-b]pyridin-4-yl}phenyl)methyl]-1,2,4-oxadiazole-3-carboxamide;
4-tert-butyl-N-[(2-fluoro-4-{3-[(piperidin-4-yl)amino]-1H-pyrazolo[3,4-b]pyridin-4-yl}phenyl)methyl]benzamide;
4-tert-butyl-N-{[2-fluoro-4-(3-{[1-(prop-2-enoyl)piperidin-4-yl]amino}-1H-pyrazolo[3,4-b]pyridin-4-yl)phenyl]methyl}benzamide;
5-tert-butyl-N-{[2-fluoro-4-(3-{[1-(prop-2-enoyl)piperidin-4-yl]amino}-1H-pyrazolo[3,4-b]pyridin-4-yl)phenyl]methyl}-1,2,4-oxadiazole-3-carboxamide;
4-tert-butyl-N-{[2-fluoro-4-(3-{[1-(prop-2-enoyl)azetidin-3-yl]amino}-1H-pyrazolo[3,4-b]pyridin-4-yl)phenyl]methyl}benzamide;
5-tert-butyl-N-{[2-fluoro-4-(3-{[1-(prop-2-enoyl)azetidin-3-yl]amino}-1H-pyrazolo[3,4-b]pyridin-4-yl)phenyl]methyl}-1,2,4-oxadiazole-3-carboxamide;
N-[(4-{3-[(3R)-3-aminopyrrolidin-1-yl]-1H-pyrazolo[3,4-b]pyridin-4-yl}-2-fluorophenyl)methyl]-5-tert-butyl-1,2,4-oxadiazole-3-carboxamide;
5-tert-butyl-N-[(2-fluoro-4-{3-[(piperidin-4-yl)amino]-1H-pyrazolo[3,4-b]pyridin-4-yl}phenyl)methyl]-1,2,4-oxadiazole-3-carboxamide;
N-{[4-(3-{[(3R)-1-(but-2-ynoyl)pyrrolidin-3-yl]amino}-1H-pyrazolo[3,4-b]pyridin-4-yl)-2-fluorophenyl]methyl}-4-(trifluoromethyl)benzamide;
N-{[4-(3-{[(3R)-1-(but-2-ynoyl)pyrrolidin-3-yl]amino}-1H-pyrazolo[3,4-b]pyridin-4-yl)-2-fluorophenyl]methyl}-5-tert-butyl-1,2-oxazole-3-carboxamide;
N-{[4-(3-{[(3R)-1-(but-2-ynoyl)pyrrolidin-3-yl]amino}-1H-pyrazolo[3,4-b]pyridin-4-yl)-2-fluorophenyl]methyl}-4-methoxybenzamide;
5-tert-butyl-N-{[4-(3-{[(3S)-1-(dimethylcarbamoyl)pyrrolidin-3-yl]amino}-1H-pyrazolo[3,4-b]pyridin-4-yl)-2-fluorophenyl]methyl}-1,2,4-oxadiazole-3-carboxamide;
(3S)-3-{[4-(4-{[(4-tert-butylphenyl)formamido]methyl}-3-fluorophenyl)-1H-pyrazolo[3,4-b]pyridin-3-yl]amino}-N,N-dimethylpyrrolidine-1-carb oxamide;
5-tert-butyl-N-{[4-(3-{[(3R)-1-(dimethylcarbamoyl)pyrrolidin-3-yl]amino}-1H-pyrazolo[3,4-b]pyridin-4-yl)-2-fluorophenyl]methyl}-1,2-oxazole-3-carboxamide;
(3R)-3-{[4-(4-{[(5-tert-butyl-1,2,4-oxadiazol-3-yl)formamido]methyl}-3-fluorophenyl)-1H-pyrazolo[3,4-b]pyridin-3-yl]amino}-N,N-dimethylpiperidine-1-carboxamide;
3-tert-butyl-N-{1-[4-(3-{[(3R)-1-(dimethylcarbamoyl)pyrrolidin-3-yl]amino}-1H-pyrazolo[3,4-b]pyridin-4-yl)phenyl]cyclopropyl}-1,2,4-oxadiazole-5-carboxamide;
5-tert-butyl-N-[(1R)-1-[4-(3-{[(3R)-1-(dimethylcarbamoyl)pyrrolidin-3-yl]amino}-1H-pyrazolo[3,4-b]pyridin-4-yl)phenyl]ethyl]-1,2,4-oxadiazole-3-carboxamide;
1-tert-butyl-N-{[4-(3-{[(3R)-1-(dimethylcarbamoyl)pyrrolidin-3-yl]amino}-1H-pyrazolo[3,4-b]pyridin-4-yl)-2-fluorophenyl]methyl}-1H-pyrazole-4-carboxamide;
3-tert-butyl-N-{[4-(3-{[(3R)-1-(dimethylcarbamoyl)pyrrolidin-3-yl]amino}-1H-pyrazolo[3,4-b]pyridin-4-yl)-2-fluorophenyl]methyl}-1,2,4-oxadiazole-5-carboxamide;
5-tert-butyl-N-{1-[2-fluoro-4-(3-{[(3R)-piperidin-3-yl]amino}-1H-pyrazolo[3,4-b]pyridin-4-yl)phenyl]cyclopropyl}-1,2,4-oxadiazole-3-carboxamide;
5-tert-butyl-N-{2-[4-(3-{[(3R)-pyrrolidin-3-yl]amino}-1H-pyrazolo[3,4-b]pyridin-4-yl)phenyl]propan-2-yl}-1,2,4-oxadiazole-3-carboxamide;
N-(5-tert-butyl-1,2,4-oxadiazol-3-yl)-2-[2-fluoro-4-(3-{[(3R)-pyrrolidin-3-yl]amino}-1H-pyrazolo[3,4-b]pyridin-4-yl)phenyl]acetamide;
N-[(4-{3-[(azetidin-3-yl)amino]-1H-pyrazolo[3,4-b]pyridin-4-yl}-2-fluorophenyl)methyl]-5-tert-butyl-1,2,4-oxadiazole-3-carboxamide;
(2R,3R)-3-{[4-(4-{[(5-tert-butyl-1,2,4-oxadiazol-3-yl)formamido]methyl}-3-fluorophenyl)-1H-pyrazolo[3,4-b]pyridin-3-yl]amino}-N,N,2-trimethylpiperidine-1-carboxamide;
5-tert-butyl-N-{[4-(3-{[(2R,3R)-1-cyclopropanecarbonyl-2-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[4-(3-{[(3R,6S)-1-cyclopropanecarbonyl-6-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[4-(3-{[(3R,6S)-1-(cyclopropanesulfonyl)-6-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[3-fluoro-4-(3-{[(3R)-pyrrolidin-3-yl]amino}-1H-pyrazolo[3,4-b]pyridin-4-yl)phenyl]methyl}-1,2,4-oxadiazole-3-carboxamide;
5-tert-butyl-N-[(2-fluoro-4-{3-[(1-propanoylazetidin-3-yl)amino]-1H-pyrazolo[3,4-b]pyridin-4-yl}phenyl)methyl]-1,2,4-oxadiazole-3-carboxamide;
N-{[4-(3-{[1-(but-2-ynoyl)azetidin-3-yl]amino}-1H-pyrazolo[3,4-b]pyridin-4-yl)-2-fluorophenyl]methyl}-5-tert-butyl-1,2,4-oxadiazole-3-carboxamide;
5-tert-butyl-N-[(4-{3-[(3R)-3-[(dimethylcarbamoyl)amino]pyrrolidin-1-yl]-1H-pyrazolo[3,4-b]pyridin-4-yl}-2-fluorophenyl)methyl]-1,2,4-oxadiazole-3-carboxamide;
5-tert-butyl-N-{1-[2-fluoro-4-(3-{[(3R)-1-(prop-2-enoyl)piperidin-3-yl]amino}-1H-pyrazolo[3,4-b]pyridin-4-yl)phenyl]cyclopropyl}-1,2,4-oxadiazole-3-carboxamide;
5-tert-butyl-N-{2-[4-(3-{[(3R)-1-(prop-2-enoyl)pyrrolidin-3-yl]amino}-1H-pyrazolo[3,4-b]pyridin-4-yl)phenyl]propan-2-yl}-1,2,4-oxadiazole-3-carboxamide;
N-(5-tert-butyl-1,2,4-oxadiazol-3-yl)-2-[2-fluoro-4-(3-{[(3R)-1-(prop-2-enoyl)pyrrolidin-3-yl]amino}-1H-pyrazolo[3,4-b]pyridin-4-yl)phenyl]acetamide;
5-tert-butyl-N-{[2-fluoro-4-(3-{[(2R,3R)-2-methyl-1-(pyrrolidine-1-carbonyl)piperidin-3-yl]amino}-1H-pyrazolo[3,4-b]pyridin-4-yl)phenyl]methyl}-1,2,4-oxadiazole-3-carboxamide;
(2R,3R)-3-{[4-(4-{[(5-tert-butyl-1,2,4-oxadiazol-3-yl)formamido]methyl}-3-fluorophenyl)-1H-pyrazolo[3,4-b]pyridin-3-yl]amino}-2-methyl-N-(2,2,2-trifluoroethyl)piperidine-1-carboxamide;
(2S,5R)-5-{[4-(4-{[(5-tert-butyl-1,2,4-oxadiazol-3-yl)formamido]methyl}-3-fluorophenyl)-1H-pyrazolo[3,4-b]pyridin-3-yl]amino}-2-methyl-N-(2,2,2-trifluoroethyl)piperidine-1-carboxamide;
5-tert-butyl-N-{[4-(3-{[(2R,3R)-1-(2-cyanoacetyl)-2-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[4-(3-{[(3R,6S)-1-(2-cyanoacetyl)-6-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[4-(3-{[(2R,3R)-1-(cyclopropanesulfonyl)-2-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[4-(3-{[(2R,3R)-1-(cyanomethyl)-2-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[4-(3-{[(3R,6S)-1-(cyanomethyl)-6-methylpiperidin-3-yl]amino}-1H-pyrazolo[3,4-b]pyridin-4-yl)-2-fluorophenyl]methyl}-1,2,4-oxadiazole-3-carboxamide;
5-tert-butyl-N-{[2-fluoro-4-(3-{[(3R,6S)-6-methylpiperidin-3-yl]amino}-1H-pyrazolo[3,4-b]pyridin-4-yl)phenyl]methyl}-1,2-oxazole-3-carboxamide;
(2S,5R)-5-{[4-(4-{[(5-tert-butyl-1,2-oxazol-3-yl)formamido]methyl}-3-fluorophenyl)-1H-pyrazolo[3,4-b]pyridin-3-yl]amino}-N,N,2-trimethylpiperidine-1-carboxamide;
5-tert-butyl-N-{[2-fluoro-4-(3-{[(3R,6S)-6-methyl-1-(prop-2-enoyl)piperidin-3-yl]amino}-1H-pyrazolo[3,4-b]pyridin-4-yl)phenyl]methyl}-1,2-oxazole-3-carboxamide;
3-tert-butyl-N-{[2-fluoro-4-(3-{[(3R,6S)-6-methylpiperidin-3-yl]amino}-1H-pyrazolo[3,4-b]pyridin-4-yl)phenyl]methyl}-1,2,4-oxadiazole-5-carboxamide;
3-tert-butyl-N-{[2-fluoro-4-(3-{[(3R,6S)-6-methyl-1-(prop-2-enoyl)piperidin-3-yl]amino}-1H-pyrazolo[3,4-b]pyridin-4-yl)phenyl]methyl}-1,2,4-oxadiazole-5-carboxamide;
N-{[2-fluoro-4-(3-{[(3R)-pyrrolidin-3-yl]amino}-1H-pyrazolo[3,4-b]pyridin-4-yl)phenyl]methyl}-5,5-dimethyl-1H,4H,5H,6H-cyclopenta[b]pyrrole-2-carboxamide;
4-tert-butyl-N-[2-(hydroxymethyl)-3-(3-{[(3R)-pyrrolidin-3-yl]amino}-1H-pyrazolo[3,4-b]pyridin-4-yl)phenyl]benzamide;
5-tert-butyl-N-{[2-fluoro-4-(3-{[(1r,4r)-4-aminocyclohexyl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(1r,4r)-4-(prop-2-enamido)cyclohexyl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(1s,4s)-4-aminocyclohexyl]amino}-1H-pyrazolo[3,4-b]pyridin-4-yl)phenyl]methyl}-1,2,4-oxadiazole-3-carboxamide;
5-tert-butyl-N-{[2-fluoro-4-(3-{[(1s,4s)-4-(prop-2-enamido)cyclohexyl]amino}-1H-pyrazolo[3,4-b]pyridin-4-yl)phenyl]methyl}-1,2,4-oxadiazole-3-carboxamide;
3-(5-tert-butyl-1,2,4-oxadiazol-3-yl)-1-[2-fluoro-4-(3-{[(3R)-pyrrolidin-3-yl]amino}-1H-pyrazolo[3,4-b]pyridin-4-yl)phenyl]urea;
3-(5-tert-butyl-1,2,4-oxadiazol-3-yl)-1-[2-fluoro-4-(3-{[(3R)-1-(prop-2-enoyl)pyrrolidin-3-yl]amino}-1H-pyrazolo[3,4-b]pyridin-4-yl)phenyl]urea;
N-{[2-fluoro-4-(3-{[(3R)-pyrrolidin-3-yl]amino}-1H-pyrazolo[3,4-b]pyridin-4-yl)phenyl]methyl}-1,5,5-trimethyl-1H,4H,5H,6H-cyclopenta[b]pyrrole-2-carboxamide;
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,5,5-trimethyl-1H,4H,5H,6H-cyclopenta[b]pyrrole-2-carboxamide;
N-[(4-{3-[(3S)-3-aminopyrrolidin-1-yl]-1H-pyrazolo[3,4-b]pyridin-4-yl}-2-fluorophenyl)methyl]-3-tert-butyl-1,2,4-oxadiazole-5-carboxamide;
5-tert-butyl-N-[2-(hydroxymethyl)-3-(3-{[(3R)-1-(prop-2-enoyl)pyrrolidin-3-yl]amino}-1H-pyrazolo[3,4-b]pyridin-4-yl)phenyl]-1,2,4-oxadiazole-3-carboxamide;
4-tert-butyl-N-[2-(hydroxymethyl)-3-(3-{[(3R)-1-(prop-2-enoyl)pyrrolidin-3-yl]amino}-1H-pyrazolo[3,4-b]pyridin-4-yl)phenyl]benzamide;
3-tert-butyl-N-{[2-fluoro-4-(3-{[(3S)-pyrrolidin-3-yl]amino}-1H-pyrazolo[3,4-b]pyridin-4-yl)phenyl]methyl}-1,2,4-oxadiazole-5-carboxamide;
3-tert-butyl-N-{[2-fluoro-4-(3-{[(3S)-1-(propan-2-yl)pyrrolidin-3-yl]amino}-1H-pyrazolo[3,4-b]pyridin-4-yl)phenyl]methyl}-1,2,4-oxadiazole-5-carboxamide;
N-{[4-(3-{[(3S)-1-acetylpyrrolidin-3-yl]amino}-1H-pyrazolo[3,4-b]pyridin-4-yl)-2-fluorophenyl]methyl}-3-tert-butyl-1,2,4-oxadiazole-5-carboxamide;
5-tert-butyl-N-[2-(hydroxymethyl)-3-(3-{[(3R)-pyrrolidin-3-yl]amino}-1H-pyrazolo[3,4-b]pyridin-4-yl)phenyl]-1,2,4-oxadiazole-3-carboxamide;
3-tert-butyl-N-{[2-fluoro-4-(3-{[(3S)-1-methylpyrrolidin-3-yl]amino}-1H-pyrazolo[3,4-b]pyridin-4-yl)phenyl]methyl}-1,2,4-oxadiazole-5-carboxamide;
3-tert-butyl-N-{[2-fluoro-4-(3-{[(3S)-piperidin-3-yl]amino}-1H-pyrazolo[3,4-b]pyridin-4-yl)phenyl]methyl}-1,2,4-oxadiazole-5-carboxamide;
N-{[4-(3-{[(3S)-1-acetylpiperidin-3-yl]amino}-1H-pyrazolo[3,4-b]pyridin-4-yl)-2-fluorophenyl]methyl}-3-tert-butyl-1,2,4-oxadiazole-5-carboxamide;
3-tert-butyl-N-{[2-fluoro-4-(3-{[(3S)-1-(prop-2-enoyl)piperidin-3-yl]amino}-1H-pyrazolo[3,4-b]pyridin-4-yl)phenyl]methyl}-1,2,4-oxadiazole-5-carboxamide;
5-ethyl-N-{[2-fluoro-4-(3-{[(2R,3R)-2-methylpiperidin-3-yl]amino}-1H-pyrazolo[3,4-b]pyridin-4-yl)phenyl]methyl}-1,2-oxazole-3-carboxamide;
(2R,3R)-3-{[4-(4-{[(5-ethyl-1,2-oxazol-3-yl)formamido]methyl}-3-fluorophenyl)-1H-pyrazolo[3,4-b]pyridin-3-yl]amino}-N,N,2-trimethylpiperidine-1-carboxamide;
N-{[2-fluoro-4-(3-{[(2R,3R)-2-methylpiperidin-3-yl]amino}-1H-pyrazolo[3,4-b]pyridin-4-yl)phenyl]methyl}-5-(propan-2-yl)-1,2-oxazole-3-carboxamide;
N-{[2-fluoro-4-(3-{[(2R,3R)-2-methyl-1-(prop-2-enoyl)piperidin-3-yl]amino}-1H-pyrazolo[3,4-b]pyridin-4-yl)phenyl]methyl}-5-(propan-2-yl)-1,2-oxazole-3-carboxamide;
5-tert-butyl-N-{[4-(3-{[(3R)-pyrrolidin-3-yl]amino}-1H-pyrazolo[3,4-b]pyridin-4-yl)phenyl]methyl}-1,2,4-oxadiazole-3-carboxamide;
5-tert-butyl-N-{[4-(3-{[(3R)-1-(prop-2-enoyl)pyrrolidin-3-yl]amino}-1H-pyrazolo[3,4-b]pyridin-4-yl)phenyl]methyl}-1,2,4-oxadiazole-3-carboxamide;
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-2-(propan-2-yl)-1,3-oxazole-5-carboxamide;
2-(dimethylamino)-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,3-oxazole-5-carboxamide;
N-({4-[3-(4-aminopiperidin-1-yl)-1H-pyrazolo[3,4-b]pyridin-4-yl]-2-fluorophenyl}methyl)-3-tert-butyl-1,2,4-oxadiazole-5-carboxamide;
3-tert-butyl-N-[(2-fluoro-4-{3-[4-(prop-2-enamido)piperidin-1-yl]-1H-pyrazolo[3,4-b]pyridin-4-yl}phenyl)methyl]-1,2,4-oxadiazole-5-carboxamide; and
2-tert-butyl-N-{[2-fluoro-4-(3-{[(3R)-pyrrolidin-3-yl]amino}-1H-pyrazolo[3,4-b]pyridin-4-yl)phenyl]methyl}-1,3-oxazole-4-carboxamide;
or a pharmaceutically acceptable salt thereof.

95. A compound selected from:
N-[(4-{3-[(3R)-3-aminopyrrolidin-1-yl]-1H-pyrazolo[3,4-b]pyridin-4-yl}-2-fluorophenyl)methyl]-3-(propan-2-yl)-1,2,4-oxadiazole-5-carboxamide
N-[(4-{3-[(3R)-3-aminopyrrolidin-1-yl]-1H-pyrazolo[3,4-b]pyridin-4-yl}-2-fluorophenyl)methyl]-3-(propan-2-yl)-1,2-oxazole-5-carboxamide
N-{[2-fluoro-4-(3-{(3R)-3-[(prop-2-enoyl)amino]pyrrolidin-1-yl}-1H-pyrazolo[3,4-b]pyridin-4-yl)phenyl]methyl}-3-(propan-2-yl)-1,2-oxazole-5-carboxamide
N-[(4-{3-[(3R)-3-acetamidopyrrolidin-1-yl]-1H-pyrazolo[3,4-b]pyridin-4-yl}-2-fluorophenyl)methyl]-3-(propan-2-yl)-1,2-oxazole-5-carboxamide
N-[(1R)-1-{4-[3-(4-aminopiperidin-1-yl)-1H-pyrazolo[3,4-b]pyridin-4-yl]-2-fluorophenyl}ethyl]-3-tert-butyl-1,2,4-oxadiazole-5-carboxamide
(2S,5R)-5-{[4-(4-{(1R)-1-[(5-tert-butyl-1,2,4-oxadiazole-3-carbonyl)amino]ethyl}-3-fluorophenyl)-1H-pyrazolo[3,4-b]pyridin-3-yl]amino}-N,N,2-trimethylpiperidine-1-carboxamide
(2S,5R)-5-[(4-{3-fluoro-4-[(1R)-1-{[5-(2-hydroxypropan-2-yl)-1,2-oxazole-3-carbonyl]amino}ethyl]phenyl}-1H-pyrazolo[3,4-b]pyridin-3-yl)amino]-N,N,2-trimethylpiperidine-1-carboxamide
(2S,5R)-5-{[4-(4-{(1R)-1-[(3-tert-butyl-1,2,4-oxadiazole-5-carbonyl)amino]ethyl}-3-fluorophenyl)-1H-pyrazolo[3,4-b]pyridin-3-yl]amino}-N,N,2-trimethylpiperidine-1-carboxamide
(2S,5R)-5-{[4-(4-{[(4,5-dimethyl-1,3-oxazole-2-carbonyl)amino]methyl}-3-fluorophenyl)-1H-pyrazolo[3,4-b]pyridin-3-yl]amino}-N,N,2-trimethylpiperidine-1-carboxamide
3-tert-butyl-N-{[2-fluoro-4-(3-{[(3R,6S)-1-(1H-imidazole-1-carbonyl)-6-methylpiperidin-3-yl]amino}-1H-pyrazolo[3,4-b]pyridin-4-yl)phenyl]methyl}-1,2,4-oxadiazole-5-carboxamide
N-[(4-{3-[(3R)-3-acetamidopyrrolidin-1-yl]-1H-pyrazolo[3,4-b]pyridin-4-yl}-2-fluorophenyl)methyl]-4,5,6,7-tetrahydro-1,3-benzoxazole-2-carboxamide
N-[(4-{3-[(3R)-3-acetamidopyrrolidin-1-yl]-1H-pyrazolo[3,4-b]pyridin-4-yl}-2-fluorophenyl)methyl]-3-(propan-2-yl)-1,2,4-oxadiazole-5-carboxamide
3-tert-butyl-N-[(1R)-1-{4-[3-(cyclopentylamino)-1H-pyrazolo[3,4-b]pyridin-4-yl]phenyl}ethyl]-1,2,4-oxadiazole-5-carboxamide
3-tert-butyl-N-[(1R)-1-{4-[3-(cyclohexylamino)-1H-pyrazolo[3,4-b]pyridin-4-yl]phenyl}ethyl]-1,2,4-oxadiazole-5-carboxamide
5-(2-hydroxypropan-2-yl)-N-{(1R)-1-[4-(3-{[1-(methanesulfonyl)piperidin-4-yl]amino}-1H-pyrazolo[3,4-b]pyridin-4-yl)phenyl]ethyl}-1,2-oxazole-3-carboxamide
N-[(1R)-1-(4-{3-[(1-acetylpiperidin-4-yl)amino]-1H-pyrazolo[3,4-b]pyridin-4-yl}phenyl)ethyl]-5-(2-hydroxypropan-2-yl)-1,2-oxazole-3-carboxamide
3-tert-butyl-N-{[4-(3-{4-[(cyclopropanecarbonyl)amino]piperidin-1-yl}-1H-pyrazolo[3,4-b]pyridin-4-yl)-2-fluorophenyl]methyl}-1,2,4-oxadiazole-5-carboxamide
N-({4-[3-(4-benzamidopiperidin-1-yl)-1H-pyrazolo[3,4-b]pyridin-4-yl]-2-fluorophenyl}methyl)-3-tert-butyl-1,2,4-oxadiazole-5-carboxamide
(2S,5R)-5-{[4-(4-{[(3-tert-butyl-1,2,4-oxadiazole-5-carbonyl)amino]methyl}-3-fluorophenyl)-1H-pyrazolo[3,4-b]pyridin-3-yl]amino}-N-ethyl-2-methylpiperidine-1-carboxamide
3-tert-butyl-N-[(1R)-1-(4-{3-[(oxolan-3-yl)amino]-1H-pyrazolo[3,4-b]pyridin-4-yl}phenyl)ethyl]-1,2,4-oxadiazole-5-carboxamide
3-tert-butyl-N-[(1R)-1-(4-{3-[(oxan-4-yl)amino]-1H-pyrazolo[3,4-b]pyridin-4-yl}phenyl)ethyl]-1,2,4-oxadiazole-5-carboxamide
3-tert-butyl-N-[(1R)-1-{4-[3-(morpholin-4-yl)-1H-pyrazolo[3,4-b]pyridin-4-yl]phenyl}ethyl]-1,2,4-oxadiazole-5-carboxamide
3-tert-butyl-N-[(1R)-1-{4-[3-(1,1-dioxo-1λ6-thiomorpholin-4-yl)-1H-pyrazolo[3,4-b]pyridin-4-yl]phenyl}ethyl]-1,2,4-oxadiazole-5-carboxamide
3-tert-butyl-N-{[4-(3-{4-[(cyclopropanesulfonyl)amino]piperidin-1-yl}-1H-pyrazolo[3,4-b]pyridin-4-yl)-2-fluorophenyl]methyl}-1,2,4-oxadiazole-5-carboxamide
methyl (2S,5R)-5-{[4-(4-{[(5-tert-butyl-1,2,4-oxadiazole-3-carbonyl)amino]methyl}-3-fluorophenyl)-1H-pyrazolo[3,4-b]pyridin-3-yl]amino}-2-methylpiperidine-1-carboxylate
5-tert-butyl-N-{[2-fluoro-4-(3-{[(3R,6S)-1-(hydroxyacetyl)-6-methylpiperidin-3-yl]amino}-1H-pyrazolo[3,4-b]pyridin-4-yl)phenyl]methyl}-1,2,4-oxadiazole-3-carboxamide
N-{[4-(3-{[(3R,6S)-1-acetyl-6-methylpiperidin-3-yl]amino}-1H-pyrazolo[3,4-b]pyridin-4-yl)-2-fluorophenyl]methyl}-5-tert-butyl-1,2,4-oxadiazole-3-carboxamide
5-tert-butyl-N-{[2-fluoro-4-(3-{[(3R,6 S)-6-methyl-1-(morpholine-4-carbonyl)piperidin-3-yl]amino}-1H-pyrazolo[3,4-b]pyridin-4-yl)phenyl]methyl}-1,2,4-oxadiazole-3-carboxamide
3-tert-butyl-N-[(1R)-1-{4-[3-(piperazin-1-yl)-1H-pyrazolo[3,4-b]pyridin-4-yl]phenyl}ethyl]-1,2,4-oxadiazole-5-carboxamide
(2S,5R)-5-[(4-{4-[(7,7-dimethyl-1-oxo-1,3,4,6,7,8-hexahydro-2H-cyclopenta[4,5]pyrrolo[1,2-a]pyrazin-2-yl)methyl]-3-fluorophenyl}-1H-pyrazolo[3,4-b]pyridin-3-yl)amino]-N,N,2-trimethylpiperidine-1-carboxamide
(2S,5R)-5-[(4-{3-fluoro-4-[(1-oxo-3,4,6,7,8,9-hexahydropyrazino[1,2-a]indol-2(1H)-yl)methyl]phenyl}-1H-pyrazolo[3,4-b]pyridin-3-yl)amino]-N,N,2-trimethylpiperidine-1-carboxamide
3-tert-butyl-N-[(2-fluoro-4-{3-[(3R)-3-(3-methyl-2-oxo-1,3-diazinan-1-yl)pyrrolidin-1-yl]-1H-pyrazolo[3,4-b]pyridin-4-yl}phenyl)methyl]-1,2,4-oxadiazole-5-carboxamide
5-[4-(4-{(1R)-1-[(3-tert-butyl-1,2,4-oxadiazole-5-carbonyl)amino]ethyl}phenyl)-1H-pyrazolo[3,4-b]pyridin-3-yl]-N,N-dimethyl-3,6-dihydropyridine-1(2H)-carboxamide
4-[4-(4-{(1R)-1-[(3-tert-butyl-1,2,4-oxadiazole-5-carbonyl)amino]ethyl}phenyl)-1H-pyrazolo[3,4-b]pyridin-3-yl]-N,N-dimethylpiperidine-1-carboxamide
3-tert-butyl-N-({2-fluoro-4-[3-(2-oxopyrrolidin-1-yl)-1H-pyrazolo[3,4-b]pyridin-4-yl]phenyl}methyl)-1,2,4-oxadiazole-5-carboxamide
5-tert-butyl-N-[(2-fluoro-4-{3-[(3R)-3-(3-methyl-2-oxo-1,3-diazinan-1-yl)pyrrolidin-1-yl]-1H-pyrazolo[3,4-b]pyridin-4-yl}phenyl)methyl]-1,2,4-oxadiazole-3-carboxamide
(2S,5R)-5-({4-[4-{[(5-tert-butyl-1,2,4-oxadiazole-3-carbonyl)amino]methyl}-2-(hydroxymethyl)phenyl]-1H-pyrazolo[3,4-b]pyridin-3-yl}amino)-N,N,2-trimethylpiperidine-1-carboxamide
(3S)-1-[4-(4-{[(5-tert-butyl-1,2,4-oxadiazole-3-carbonyl)amino]methyl}-3-fluorophenyl)-1H-pyrazolo[3,4-b]pyridin-3-yl]piperidine-3-carboxylic acid
5-[4-(4-{(1R)-1-[(5-tert-butyl-1,2,4-oxadiazole-3-carbonyl)amino]ethyl}phenyl)-1H-pyrazolo[3,4-b]pyridin-3-yl]-N,N-dimethyl-3,6-dihydropyridine-1(2H)-carboxamide
3-tert-butyl-N-{[4-(3-cyclopropyl-1H-pyrazolo[3,4-b]pyridin-4-yl)-2-fluorophenyl]methyl}-1,2,4-oxadiazole-5-carboxamide
(2S,5R)-5-{[4-(4-{[(5-tert-butyl-1,2,4-oxadiazole-3-carbonyl)amino]methyl}phenyl)-1H-pyrazolo[3,4-b]pyridin-3-yl]amino}-N,N,2-trimethylpiperidine-1-carboxamide
5-tert-butyl-N-({4-[3-(cyclopentylamino)-1H-pyrazolo[3,4-b]pyridin-4-yl]-2-fluorophenyl}methyl)-1,2,4-oxadiazole-3-carboxamide
4-tert-butyl-N-(3-{3-[(pyrrolidin-3-yl)amino]-1H-pyrazolo[3,4-b]pyridin-4-yl}phenyl)benzamide
4-tert-butyl-N-[3-(3-{[(3R)-1-(prop-2-enoyl)pyrrolidin-3-yl]amino}-1H-pyrazolo[3,4-b]pyridin-4-yl)phenyl]benzamide
5-tert-butyl-N-(3-{3-[(pyrrolidin-3-yl)amino]-1H-pyrazolo[3,4-b]pyridin-4-yl}phenyl)-1,2,4-oxadiazole-3-carboxamide
5-tert-butyl-N-[3-(3-{[(3R)-1-(prop-2-enoyl)pyrrolidin-3-yl]amino}-1H-pyrazolo[3,4-b]pyridin-4-yl)phenyl]-1,2,4-oxadiazole-3-carboxamide
5-tert-butyl-N-[2-methyl-3-(3-{[(3R)-pyrrolidin-3-yl]amino}-1H-pyrazolo[3,4-b]pyridin-4-yl)phenyl]-1,2,4-oxadiazole-3-carboxamide
5-tert-butyl-N-[2-methyl-3-(3-{[(3R)-1-(prop-2-enoyl)pyrrolidin-3-yl]amino}-1H-pyrazolo[3,4-b]pyridin-4-yl)phenyl]-1,2,4-oxadiazole-3-carboxamide
4-tert-butyl-N-[(5-{3-[(pyrrolidin-3-yl)amino]-1H-pyrazolo[3,4-b]pyridin-4-yl}pyridin-2-yl)methyl]benzamide
5-tert-butyl-N-[(2-fluoro-4-{3-[(pyrrolidin-3-yl)oxy]-1H-pyrazolo[3,4-b]pyridin-4-yl}phenyl)methyl]-1,2,4-oxadiazole-3-carboxamide
5-tert-butyl-N-{1-[4-(3-{[(3R)-1-(prop-2-enoyl)piperidin-3-yl]amino}-1H-pyrazolo[3,4-b]pyridin-4-yl)phenyl]cyclopropyl}-1,2,4-oxadiazole-3-carboxamide
5-tert-butyl-N-[1-(4-{3-[(1-formylpiperidin-3-yl)amino]-1H-pyrazolo[3,4-b]pyridin-4-yl}phenyl)cyclopropyl]-1,2,4-oxadiazole-3-carboxamide
5-tert-butyl-N-{[2-fluoro-4-(3-{[(3R)-1-(prop-2-enoyl)pyrrolidin-3-yl]oxy}-1H-pyrazolo[3,4-b]pyridin-4-yl)phenyl]methyl}-1,2,4-oxadiazole-3-carboxamide
5-tert-butyl-N-[(2-fluoro-4-{3-[(1-formylpyrrolidin-3-yl)oxy]-1H-pyrazolo[3,4-b]pyridin-4-yl}phenyl)methyl]-1,2,4-oxadiazole-3-carboxamide
5-tert-butyl-N-[(2-fluoro-4-{3-[(pyrrolidin-3-yl)methyl]-1H-pyrazolo[3,4-b]pyridin-4-yl}phenyl)methyl]-1,2,4-oxadiazole-3-carboxamide
5-tert-butyl-N-{[2-fluoro-4-(3-{[1-(prop-2-enoyl)pyrrolidin-3-yl]methyl}-1H-pyrazolo[3,4-b]pyridin-4-yl)phenyl]methyl}-1,2,4-oxadiazole-3-carboxamide
5-tert-butyl-N-({2-fluoro-4-[3-(pyrrolidin-3-yl)-1H-pyrazolo[3,4-b]pyridin-4-yl]phenyl}methyl)-1,2,4-oxadiazole-3-carboxamide
5-tert-butyl-N-[(2-fluoro-4-{3-[1-(prop-2-enoyl)pyrrolidin-3-yl]-1H-pyrazolo[3,4-b]pyridin-4-yl}phenyl)methyl]-1,2,4-oxadiazole-3-carboxamide
5-tert-butyl-N-({4-[3-(2,5-dihydro-1H-pyrrol-3-yl)-1H-pyrazolo[3,4-b]pyridin-4-yl]-2-fluorophenyl}methyl)-1,2,4-oxadiazole-3-carboxamide
5-tert-butyl-N-[(2-fluoro-4-{3-[1-(prop-2-enoyl)-2,5-dihydro-1H-pyrrol-3-yl]-1H-pyrazolo[3,4-b]pyridin-4-yl}phenyl)methyl]-1,2,4-oxadiazole-3-carboxamide
5-tert-butyl-N-{[4-(3-{[(3R)-1-(dimethylcarbamoyl)pyrrolidin-3-yl]oxy}-1H-pyrazolo[3,4-b]pyridin-4-yl)-2-fluorophenyl]methyl}-1,2,4-oxadiazole-3-carboxamide
3-tert-butyl-N-[(2-fluoro-4-{3-[(pyrrolidin-3-yl)oxy]-1H-pyrazolo[3,4-b]pyridin-4-yl}phenyl)methyl]-1,2,4-oxadiazole-5-carboxamide
N-[(4-{3-[(1-acetylpyrrolidin-3-yl)oxy]-1H-pyrazolo[3,4-b]pyridin-4-yl}-2-fluorophenyl)methyl]-3-tert-butyl-1,2,4-oxadiazole-5-carboxamide
2-(7,7-dimethyl-1-oxo-1,3,4,6,7,8-hexahydro-2H-cyclopenta[4,5]pyrrolo[1,2-a]pyrazin-2-yl)-4-{3-[(pyrrolidin-3-yl)amino]-1H-pyrazolo[3,4-b]pyridin-4-yl}pyridine-3-carbaldehyde
2-(7,7-dimethyl-1-oxo-1,3,4,6,7,8-hexahydro-2H-cyclopenta[4,5]pyrrolo[1,2-a]pyrazin-2-yl)-4-(3-{[1-(prop-2-enoyl)pyrrolidin-3-yl]amino}-1H-pyrazolo[3,4-b]pyridin-4-yl)pyridine-3-carbaldehyde
5-tert-butyl-N-(3-methyl-4-{3-[(pyrrolidin-3-yl)amino]-1H-pyrazolo[3,4-b]pyridin-4-yl}pyridin-2-yl)-1,2,4-oxadiazole-3-carboxamide
5-tert-butyl-N-[3-methyl-4-(3-{[(3R)-1-(prop-2-enoyl)pyrrolidin-3-yl]amino}-1H-pyrazolo[3,4-b]pyridin-4-yl)pyridin-2-yl]-1,2,4-oxadiazole-3-carboxamide
4-[4-({[(5-tert-butyl-1,2,4-oxadiazol-3-yl)methyl]amino}methyl)-3-fluorophenyl]-N-(pyrrolidin-3-yl)-1H-pyrazolo[3,4-b]pyridin-3-amine
5-tert-butyl-N-{(1S)-1-[2-fluoro-4-(3-{[(3R)-pyrrolidin-3-yl]amino}-1H-pyrazolo[3,4-b]pyridin-4-yl)phenyl]ethyl}-1,2,4-oxadiazole-3-carboxamide
5-tert-butyl-N-{(1S)-1-[4-(3-{[(3R)-1-(dimethylcarbamoyl)pyrrolidin-3-yl]amino}-1H-pyrazolo[3,4-b]pyridin-4-yl)-2-fluorophenyl]ethyl}-1,2,4-oxadiazole-3-carboxamide
N-{[2-fluoro-4-(3-{[(2R,3R)-2-methylpiperidin-3-yl]amino}-1H-pyrazolo[3,4-b]pyridin-4-yl)phenyl]methyl}-5-(2-methylpropyl)-1,2-oxazole-3-carboxamide
N-{[2-fluoro-4-(3-{[(2R,3R)-2-methyl-1-(prop-2-enoyl)piperidin-3-yl]amino}-1H-pyrazolo[3,4-b]pyridin-4-yl)phenyl]methyl}-5-(2-methylpropyl)-1,2-oxazole-3-carboxamide
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-5-(2-hydroxypropan-2-yl)-1,2-oxazole-3-carboxamide
2-tert-butyl-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,3-oxazole-4-carboxamide
2-tert-butyl-N-{[4-(3-{[(3R)-1-(dimethylcarbamoyl)pyrrolidin-3-yl]amino}-1H-pyrazolo[3,4-b]pyridin-4-yl)-2-fluorophenyl]methyl}-1,3-oxazole-4-carboxamide
2-[3-(hydroxymethyl)-4-{3-[(pyrrolidin-3-yl)amino]-1H-pyrazolo[3,4-b]pyridin-4-yl}pyridin-2-yl]-7,7-dimethyl-3,4,7,8-tetrahydro-2H-cyclopenta[4,5]pyrrolo[1,2-a]pyrazin-1 (6H)-one
2-[3-(hydroxymethyl)-4-(3-{[(3R)-1-(prop-2-enoyl)pyrrolidin-3-yl]amino}-1H-pyrazolo[3,4-b]pyridin-4-yl)pyridin-2-yl]-7,7-dimethyl-3,4,7,8-tetrahydro-2H-cyclopenta[4,5]pyrrolo[1,2-a]pyrazin-1 (6H)-one
(3R)-3-({4-[2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl]-1H-pyrazolo[3,4-b]pyridin-3-yl}amino)-N,N-dimethylpyrrolidine-1-carboxamide
4-tert-butyl-N-[3-methyl-4-(3-{[1-(prop-2-enoyl)pyrrolidin-3-yl]amino}-1H-pyrazolo[3,4-b]pyridin-4-yl)pyridin-2-yl]benzamide
N-[(5-tert-butyl-1,2,4-oxadiazol-3-yl)methyl]-2-fluoro-4-{3-[(pyrrolidin-3-yl)amino]-1H-pyrazolo[3,4-b]pyridin-4-yl}benzamide
N-[(5-tert-butyl-1,2,4-oxadiazol-3-yl)methyl]-2-fluoro-4-(3-{[(3R)-1-(prop-2-enoyl)pyrrolidin-3-yl]amino}-1H-pyrazolo[3,4-b]pyridin-4-yl)benzamide
5-tert-butyl-N-{(1S)-1-[2-fluoro-4-(3-{[(3R)-1-(prop-2-enoyl)pyrrolidin-3-yl]amino}-1H-pyrazolo[3,4-b]pyridin-4-yl)phenyl]ethyl}-1,2,4-oxadiazole-3-carboxamide
5-ethyl-N-{[2-fluoro-4-(3-{[(2R,3R)-2-methyl-1-(prop-2-enoyl)piperidin-3-yl]amino}-1H-pyrazolo[3,4-b]pyridin-4-yl)phenyl]methyl}-1,2-oxazole-3-carboxamide
(2R,3R)-3-({4-[3-fluoro-4-({[5-(propan-2-yl)-1,2-oxazole-3-carbonyl]amino}methyl)phenyl]-1H-pyrazolo[3,4-b]pyridin-3-yl}amino)-N,N,2-trimethylpiperidine-1-carboxamide
(2R,3R)-3-({4-[3-fluoro-4-({[5-(2-methylpropyl)-1,2-oxazole-3-carbonyl]amino}methyl)phenyl]-1H-pyrazolo[3,4-b]pyridin-3-yl}amino)-N,N,2-trimethylpiperidine-1-carboxamide
N-({4-[3-(3-aminopyrrolidin-1-yl)-1H-pyrazolo[3,4-b]pyridin-4-yl]-2-fluorophenyl}methyl)-5-tert-butyl-1,2-oxazole-3-carboxamide
5-tert-butyl-N-{[2-fluoro-4-(3-{(3R)-3-[(prop-2-enoyl)amino]pyrrolidin-1-yl}-1H-pyrazolo[3,4-b]pyridin-4-yl)phenyl]methyl}-1,2-oxazole-3-carboxamide
5-tert-butyl-N-[(4-{3-[(3R)-3-{[(2E)-4-(dimethylamino)but-2-enoyl]amino}pyrrolidin-1-yl]-1H-pyrazolo[3,4-b]pyridin-4-yl}-2-fluorophenyl)methyl]-1,2-oxazole-3-carboxamide
1-[3-({4-[4-({[(5-tert-butyl-1,2,4-oxadiazol-3-yl)methyl]amino}methyl)-3-fluorophenyl]-1H-pyrazolo[3,4-b]pyridin-3-yl}amino)pyrrolidin-1-yl]prop-2-en-1-one
N-{[2-fluoro-4-(3-{[1-(prop-2-enoyl)pyrrolidin-3-yl]amino}-1H-pyrazolo[3,4-b]pyridin-4-yl)phenyl]methyl}methanesulfonamide
N-{[4-(3-{[(1R,2R)-2-aminocyclohexyl]amino}-1H-pyrazolo[3,4-b]pyridin-4-yl)-2-fluorophenyl]methyl}-5-tert-butyl-1,2-oxazole-3-carboxamide
5-tert-butyl-N-({2-fluoro-4-[3-({(1R,2R)-2-[(prop-2-enoyl)amino]cyclohexyl}amino)-1H-pyrazolo[3,4-b]pyridin-4-yl]phenyl}methyl)-1,2-oxazole-3-carboxamide
N-{[4-(3-{[(1R,3 S)-3-aminocyclopentyl]amino}-1H-pyrazolo[3,4-b]pyridin-4-yl)-2-fluorophenyl]methyl}-5-tert-butyl-1,2,4-oxadiazole-3-carboxamide
(2R,3R)-3-({4-[3-fluoro-4-({[5-(2-hydroxypropan-2-yl)-1,2-oxazole-3-carbonyl]amino}methyl)phenyl]-1H-pyrazolo[3,4-b]pyridin-3-yl}amino)-N,N,2-trimethylpiperidine-1-carboxamide
(2R,3R)-3-({4-[4-({[5-(2-aminopropan-2-yl)-1,2-oxazole-3-carbonyl]amino}methyl)-3-fluorophenyl]-1H-pyrazolo[3,4-b]pyridin-3-yl}amino)-N,N,2-trimethylpiperidine-1-carboxamide
5-tert-butyl-N-({2-fluoro-4-[3-({(1 S,2 S)-2-[(prop-2-enoyl)amino]cyclohexyl}amino)-1H-pyrazolo[3,4-b]pyridin-4-yl]phenyl}methyl)-1,2-oxazole-3-carboxamide
5-tert-butyl-N-({2-fluoro-4-[3-({(1R,3 S)-3-[(prop-2-enoyl)amino]cyclopentyl}amino)-1H-pyrazolo[3,4-b]pyridin-4-yl]phenyl}methyl)-1,2,4-oxadiazole-3-carboxamide
N-[(4-{3-[(2S,5R)-5-amino-2-methylpiperidin-1-yl]-1H-pyrazolo[3,4-b]pyridin-4-yl}-2-fluorophenyl)methyl]-5-tert-butyl-1,2-oxazole-3-carboxamide
5-tert-butyl-N-{[2-fluoro-4-(3-{(2 S, 5R)-2-methyl-5-[(prop-2-enoyl)amino]piperidin-1-yl}-1H-pyrazolo[3,4-b]pyridin-4-yl)phenyl]methyl}-1,2-oxazole-3-carboxamide
N-{[2-fluoro-4-(3-{(3R)-3-[(prop-2-enoyl)amino]pyrrolidin-1-yl}-1H-pyrazolo[3,4-b]pyridin-4-yl)phenyl]methyl}-4-(2-hydroxypropan-2-yl)benzamide
2-cyclobutyl-N-{[4-(3-{[(3R)-1-(dimethylcarbamoyl)pyrrolidin-3-yl]amino}-1H-pyrazolo[3,4-b]pyridin-4-yl)-2-fluorophenyl]methyl}-1,3-oxazole-4-carboxamide
2-cyclopropyl-N-{[4-(3-{[(3R)-1-(dimethylcarbamoyl)pyrrolidin-3-yl]amino}-1H-pyrazolo[3,4-b]pyridin-4-yl)-2-fluorophenyl]methyl}-1,3-oxazole-4-carboxamide
N-({4-[3-(3-aminopyrrolidin-1-yl)-1H-pyrazolo[3,4-b]pyridin-4-yl]-2-fluorophenyl}methyl)-3-tert-butyl-1,2,4-oxadiazole-5-carboxamide
3-tert-butyl-N-{[2-fluoro-4-(3-{(3R)-3-[(prop-2-enoyl)amino]pyrrolidin-1-yl}-1H-pyrazolo[3,4-b]pyridin-4-yl)phenyl]methyl}-1,2,4-oxadiazole-5-carboxamide
3-tert-butyl-N-[(2-fluoro-4-{3-[3-(methylamino)pyrrolidin-1-yl]-1H-pyrazolo[3,4-b]pyridin-4-yl}phenyl)methyl]-1,2,4-oxadiazole-5-carboxamide
N-({4-[3-(3-aminopyrrolidin-1-yl)-1H-pyrazolo[3,4-b]pyridin-4-yl]-2-fluorophenyl}methyl)-3-tert-butyl-N-methyl-1,2,4-oxadiazole-5-carboxamide
3-tert-butyl-N-{[2-fluoro-4-(3-{(3R)-3-[(prop-2-enoyl)amino]pyrrolidin-1-yl}-1H-pyrazolo[3,4-b]pyridin-4-yl)phenyl]methyl}-N-methyl-1,2,4-oxadiazole-5-carboxamide
3-tert-butyl-N-[(2-fluoro-4-{3-[3-(methylamino)pyrrolidin-1-yl]-1H-pyrazolo[3,4-b]pyridin-4-yl}phenyl)methyl]-N-methyl-1,2,4-oxadiazole-5-carboxamide
3-tert-butyl-N-{[2-fluoro-4-(3-{(3R)-3-[methyl(prop-2-enoyl)amino]pyrrolidin-1-yl}-1H-pyrazolo[3,4-b]pyridin-4-yl)phenyl]methy}-N-methyl-1,2,4-oxadiazole-5-carboxamide
5-chloro-N-[(2-fluoro-4-{3-[(pyrrolidin-3-yl)amino]-1H-pyrazolo[3,4-b]pyridin-4-yl}phenyl)methyl]-1,2-oxazole-3-carboxamide
5-chloro-N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,2-oxazole-3-carboxamide
5-chloro-N-{[4-(3-{[(3R)-1-(dimethylcarbamoyl)pyrrolidin-3-yl]amino}-1H-pyrazolo[3,4-b]pyridin-4-yl)-2-fluorophenyl]methyl}-1,2-oxazole-3-carboxamide
N-[(2-fluoro-4-{3-[(pyrrolidin-3-yl)amino]-1H-pyrazolo[3,4-b]pyridin-4-yl}phenyl)methyl]-5-phenyl-1,2-oxazole-3-carboxamide
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-5-phenyl-1,2-oxazole-3-carboxamide
N-{[4-(3-{[(3R)-1-(dimethylcarbamoyl)pyrrolidin-3-yl]amino}-1H-pyrazolo[3,4-b]pyridin-4-yl)-2-fluorophenyl]methyl}-5-phenyl-1,2-oxazole-3-carboxamide
3-tert-butyl-N-{[2-fluoro-4-(3-{(3R)-3-[methyl(prop-2-enoyl)amino]pyrrolidin-1-yl}-1H-pyrazolo[3,4-b]pyridin-4-yl)phenyl]methyl}-1,2,4-oxadiazole-5-carboxamide
N-[(1R)-1-(4-{3-[(3R)-3-acetamidopyrrolidin-1-yl]-1H-pyrazolo[3,4-b]pyridin-4-yl}-2-fluorophenyl)ethyl]-5-(2-hydroxypropan-2-yl)-1,2-oxazole-3-carboxamide
N-[(2-fluoro-4-{3-[(pyrrolidin-3-yl)amino]-1H-pyrazolo[3,4-b]pyridin-4-yl}phenyl)methyl]-3,4-dihydro-2H-1,5-benzodioxepine-7-carboxamide
N-{[2-fluoro-4-(3-{[1-(prop-2-enoyl)pyrrolidin-3-yl]amino}-1H-pyrazolo[3,4-b]pyridin-4-yl)phenyl]methyl}-3,4-dihydro-2H-1,5-benzodioxepine-7-carboxamide
N-[(2-fluoro-4-{3-[(pyrrolidin-3-yl)amino]-1H-pyrazolo[3,4-b]pyridin-4-yl}phenyl)methyl]-1,3-benzothiazole-5-carboxamide
N-{(1R)-1-[2-fluoro-4-(3-{(3R)-3-[(prop-2-enoyl)amino]pyrrolidin-1-yl}-1H-pyrazolo[3,4-b]pyridin-4-yl)phenyl]ethyl}-5-(2-hydroxypropan-2-yl)-1,2-oxazole-3-carboxamide
N-{[2-fluoro-4-(3-{(3R)-3-[(prop-2-enoyl)amino]pyrrolidin-1-yl}-1H-pyrazolo[3,4-b]pyridin-4-yl)phenyl]methyl}-4,5,6,7-tetrahydro-1,3-benzothiazole-2-carboxamide
N-[(4-{3-[(3R)-3-acetamidopyrrolidin-1-yl]-1H-pyrazolo[3,4-b]pyridin-4-yl}-2-fluorophenyl)methyl]-4,5,6,7-tetrahydro-1,3-benzothiazole-2-carboxamide
N-[(4-{3-[(3R)-3-aminopyrrolidin-1-yl]-1H-pyrazolo[3,4-b]pyridin-4-yl}-2-fluorophenyl)methyl]-2-(propan-2-yl)-1,3-oxazole-4-carboxamide
N-{[2-fluoro-4-(3-{(3R)-3-[(prop-2-enoyl)amino]pyrrolidin-1-yl}-1H-pyrazolo[3,4-b]pyridin-4-yl)phenyl]methyl}-2-(propan-2-yl)-1,3-oxazole-4-carboxamide
N-[(4-{3-[(3R)-3-acetamidopyrrolidin-1-yl]-1H-pyrazolo[3,4-b]pyridin-4-yl}-2-fluorophenyl)methyl]-2-(propan-2-yl)-1,3-oxazole-4-carboxamide
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-1,3-benzothiazole-5-carboxamide
N-[(1R)-1-(4-{3-[(3R)-3-acetamidopyrrolidin-1-yl]-1H-pyrazolo[3,4-b]pyridin-4-yl}phenyl)ethyl]-5-(2-hydroxypropan-2-yl)-1,2-oxazole-3-carboxamide
5-(2-hydroxypropan-2-yl)-N-{(1R)-1-[4-(3-{4-[(prop-2-enoyl)amino]piperidin-1-yl}-1H-pyrazolo[3,4-b]pyridin-4-yl)phenyl]ethyl}-1,2-oxazole-3-carboxamide
N-[(1R)-1-{4-[3-(4-acetamidopiperidin-1-yl)-1H-pyrazolo[3,4-b]pyridin-4-yl]phenyl}ethyl]-5-(2-hydroxypropan-2-yl)-1,2-oxazole-3-carboxamide
N-[(4-{3-[(3R)-3-aminopyrrolidin-1-yl]-1H-pyrazolo[3,4-b]pyridin-4-yl}-2-fluorophenyl)methyl]-4,5,6,7-tetrahydro-1,3-benzothiazole-2-carboxamide
N-{[2-fluoro-4-(3-{(3R)-3-[(prop-2-enoyl)amino]pyrrolidin-1-yl}-1H-pyrazolo[3,4-b]pyridin-4-yl)phenyl]methyl}-5-(propan-2-yl)-1,2,4-oxadiazole-3-carboxamide
N-[(4-{3-[(3R)-3-acetamidopyrrolidin-1-yl]-1H-pyrazolo[3,4-b]pyridin-4-yl}-2-fluorophenyl)methyl]-5-(propan-2-yl)-1,2,4-oxadiazole-3-carboxamide
N-[(4-{3-[(3R)-3-aminopyrrolidin-1-yl]-1H-pyrazolo[3,4-b]pyridin-4-yl}-2-fluorophenyl)methyl]-5-(propan-2-yl)-1,2,4-oxadiazole-3-carboxamide
N-[(1R)-1-{4-[3-(4-acetamidopiperidin-1-yl)-1H-pyrazolo[3,4-b]pyridin-4-yl]phenyl}ethyl]-3-tert-butyl-1,2,4-oxadiazole-5-carboxamide
5-tert-butyl-N-{(1R)-1-[2-fluoro-4-(3-{[(3R)-1-(prop-2-enoyl)pyrrolidin-3-yl]amino}-1H-pyrazolo[3,4-b]pyridin-4-yl)phenyl]ethyl}-1,2,4-oxadiazole-3-carboxamide
N-[(4-{3-[(3R)-3-acetamidopyrrolidin-1-yl]-1H-pyrazolo[3,4-b]pyridin-4-yl}-2-fluorophenyl)methyl]-3-tert-butyl-1,2,4-oxadiazole-5-carboxamide
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-2,3-dihydro-1,4-benzodioxine-6-carboxamide
N-{[2-fluoro-4-(3-{[(3R)-1-(prop-2-enoyl)pyrrolidin-3-yl]amino}-1H-pyrazolo[3,4-b]pyridin-4-yl)phenyl]methyl}piperidine-1-carboxamide
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-3-methyl-6,7-dihydro-5H-pyrazolo[5,1-b][1,3]oxazine-2-carboxamide
N-{[2-fluoro-4-(3-{[(3R)-1-(prop-2-enoyl)pyrrolidin-3-yl]amino}-1H-pyrazolo[3,4-b]pyridin-4-yl)phenyl]methyl}imidazo[1,2-a]pyridine-2-carboxamide
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-2-methyl-1,3-benzothiazole-5-carboxamide
N-{[2-fluoro-4-(3-{[1-(prop-2-enoyl)pyrrolidin-3-yl]amino}-1H-pyrazolo[3,4-b]pyridin-4-yl)phenyl]methyl}-1,3-benzoxazole-5-carboxamide
N-{[2-fluoro-4-(3-{[1-(prop-2-enoyl)pyrrolidin-3-yl]amino}-1H-pyrazolo[3,4-b]pyridin-4-yl)phenyl]methyl}-2-methyl-1,3-benzoxazole-5-carboxamide
3-tert-butyl-N-({2-fluoro-4-[3-(4-methyl-2,3-dioxopiperazin-1-yl)-1H-pyrazolo[3,4-b]pyridin-4-yl]phenyl}methyl)-1,2,4-oxadiazole-5-carboxamide
5-(2-hydroxypropan-2-yl)-N-{(R)-1-[4-(3-{(3R)-3-[(prop-2-enoyl)amino]pyrrolidin-1-yl}-1H-pyrazolo[3,4-b]pyridin-4-yl)phenyl]ethyl}-1,2-oxazole-3-carboxamide
3-tert-butyl-N-[(1R)-1-{4-[3-(piperidin-1-yl)-1H-pyrazolo[3,4-b]pyridin-4-yl]phenyl}ethyl]-1,2,4-oxadiazole-5-carboxamide
N-[(1R)-1-(4-{3-[(1,1-dioxo-1λ6-thian-4-yl)amino]-1H-pyrazolo[3,4-b]pyridin-4-yl}phenyl)ethyl]-5-(2-hydroxypropan-2-yl)-1,2-oxazole-3-carboxamide
3-tert-butyl-N-{(1R)-1-[4-(3-{[1-(methanesulfonyl)pyrrolidin-3-yl]amino}-1H-pyrazolo[3,4-b]pyridin-4-yl)phenyl]ethyl}-1,2,4-oxadiazole-5-carboxamide
3-tert-butyl-N-{(1R)-1-[4-(3-{[1-(methanesulfonyl)piperidin-4-yl]amino}-1H-pyrazolo[3,4-b]pyridin-4-yl)phenyl]ethyl}-1,2,4-oxadiazole-5-carboxamide
N-[(1R)-1-{4-[3-(morpholin-4-yl)-1H-pyrazolo[3,4-b]pyridin-4-yl]phenyl}ethyl]-3-(propan-2-yl)-1,2,4-oxadiazole-5-carboxamide
N-{[2-fluoro-4-(3-{[(3R)-1-(prop-2-enoyl)pyrrolidin-3-yl]amino}-1H-pyrazolo[3,4-b]pyridin-4-yl)phenyl]methyl}-5-methylimidazo[1,2-a]pyridine-2-carboxamide
(2S,5R)-5-{[4-(4-{[(3-tert-butyl-1,2,4-oxadiazole-5-carbonyl)amino]methyl}-3-fluorophenyl)-1H-pyrazolo[3,4-b]pyridin-3-yl]amino}-N,N,2-trimethylpiperidine-1-carboxamide
3-tert-butyl-N-{[2-fluoro-4-(3-{[(3S)-1-methyl-5-oxopyrrolidin-3-yl]amino}-1H-pyrazolo[3,4-b]pyridin-4-yl)phenyl]methyl}-1,2,4-oxadiazole-5-carboxamide
N-[(4-{3-[(3R)-3-aminopyrrolidin-1-yl]-1H-pyrazolo[3,4-b]pyridin-4-yl}-2-fluorophenyl)methyl]-1,3-benzoxazole-2-carboxamide
N-[(4-{3-[(3R)-3-acetamidopyrrolidin-1-yl]-1H-pyrazolo[3,4-b]pyridin-4-yl}-2-fluorophenyl)methyl]-1,3-benzoxazole-2-carboxamide
N-{[2-fluoro-4-(3-{(3R)-3-[(prop-2-enoyl)amino]pyrrolidin-1-yl}-1H-pyrazolo[3,4-b]pyridin-4-yl)phenyl]methyl}-1,3-benzoxazole-2-carboxamide
3-tert-butyl-N-{(1R)-1-[4-(3-{4-[(methanesulfonyl)amino]piperidin-1-yl}-1H-pyrazolo[3,4-b]pyridin-4-yl)phenyl]ethyl}-1,2,4-oxadiazole-5-carboxamide
N-[(1R)-1-{4-[3-(cyclopentylamino)-1H-pyrazolo[3,4-b]pyridin-4-yl]phenyl}ethyl]-5-(2-hydroxypropan-2-yl)-1,2-oxazole-3-carboxamide
5-(2-hydroxypropan-2-yl)-N-[(1R)-1-(4-{3-[(oxolan-3-yl)amino]-1H-pyrazolo[3,4-b]pyridin-4-yl}phenyl)ethyl]-1,2-oxazole-3-carboxamide
N-[(1R)-1-{4-[3-(cyclohexylamino)-1H-pyrazolo[3,4-b]pyridin-4-yl]phenyl}ethyl]-5-(2-hydroxypropan-2-yl)-1,2-oxazole-3-carboxamide
5-(2-hydroxypropan-2-yl)-N-[(1R)-1-(4-{3-[(oxan-4-yl)amino]-1H-pyrazolo[3,4-b]pyridin-4-yl}phenyl)ethyl]-1,2-oxazole-3-carboxamide
N-[(4-{3-[(3R)-3-aminopyrrolidin-1-yl]-1H-pyrazolo[3,4-b]pyridin-4-yl}-2-fluorophenyl)methyl]-5-(2-cyanopropan-2-yl)-1,2-oxazole-3-carboxamide
5-(2-cyanopropan-2-yl)-N-{[2-fluoro-4-(3-{(3R)-3-[(prop-2-enoyl)amino]pyrrolidin-1-yl}-1H-pyrazolo[3,4-b]pyridin-4-yl)phenyl]methyl}-1,2-oxazole-3-carboxamide
N-[(4-{3-[(3R)-3-acetamidopyrrolidin-1-yl]-1H-pyrazolo[3,4-b]pyridin-4-yl}-2-fluorophenyl)methyl]-5-(2-cyanopropan-2-yl)-1,2-oxazole-3-carboxamide
5-(2-hydroxypropan-2-yl)-N-[(1R)-1-{4-[3-(morpholin-4-yl)-1H-pyrazolo[3,4-b]pyridin-4-yl]phenyl}ethyl]-1,2-oxazole-3-carboxamide
N-[(1R)-1-{4-[3-(1,1-dioxo-1λ6-thiomorpholin-4-yl)-1H-pyrazolo[3,4-b]pyridin-4-yl]phenyl}ethyl]-3-(propan-2-yl)-1,2,4-oxadiazole-5-carboxamide
N-[(1R)-1-{4-[3-(1,1-dioxo-1λ6-thiomorpholin-4-yl)-1H-pyrazolo[3,4-b]pyridin-4-yl]phenyl}ethyl]-5-(2-hydroxypropan-2-yl)-1,2-oxazole-3-carboxamide
N-{[4-(3-{[(3R,6S)-1-acetyl-6-methylpiperidin-3-yl]amino}-1H-pyrazolo[3,4-b]pyridin-4-yl)-2-fluorophenyl]methyl}-3-tert-butyl-1,2,4-oxadiazole-5-carboxamide
3-tert-butyl-N-{[2-fluoro-4-(3-{[(3R,6S)-6-methyl-1-(morpholine-4-carbonyl)piperidin-3-yl]amino}-1H-pyrazolo[3,4-b]pyridin-4-yl)phenyl]methyl}-1,2,4-oxadiazole-5-carboxamide
methyl (2S,5R)-5-{[4-(4-{[(3-tert-butyl-1,2,4-oxadiazole-5-carbonyl)amino]methyl}-3-fluorophenyl)-1H-pyrazolo[3,4-b]pyridin-3-yl]amino}-2-methylpiperidine-1-carboxylate
3-tert-butyl-N-{[2-fluoro-4-(3-{[(3R,6S)-6-methyl-1-(oxetan-3-yl)piperidin-3-yl]amino}-1H-pyrazolo[3,4-b]pyridin-4-yl)phenyl]methyl}-1,2,4-oxadiazole-5-carboxamide
(2 S,5R)-5-{[4-(3-fluoro-4-{[2-fluoro-4-(2-hydroxypropan-2-yl)benzamido]methyl}phenyl)-1H-pyrazolo[3,4-b]pyridin-3-yl]amino}-N,N,2-trimethylpiperidine-1-carboxamide
N-{[4-(3-{[(3R,6S)-1-(dimethylcarbamoyl)-6-methylpiperidin-3-yl]amino}-1H-pyrazolo[3,4-b]pyridin-4-yl)-2-fluorophenyl]methyl}-4,5,6,7-tetrahydro-1,3-benzothiazole-2-carboxamide
(2S,5R)-5-({4-[3-fluoro-4-({[5-(2-hydroxypropan-2-yl)-1,2-oxazole-3-carbonyl]amino}methyl)phenyl]-1H-pyrazolo[3,4-b]pyridin-3-yl}amino)-N,N,2-trimethylpiperidine-1-carboxamide
3-tert-butyl-N-{[2-fluoro-4-(3-{[(3R,6S)-6-methyl-1-(3-methyloxetane-3-carbonyl)piperidin-3-yl]amino}-1H-pyrazolo[3,4-b]pyridin-4-yl)phenyl]methyl}-1,2,4-oxadiazole-5-carboxamide
3-tert-butyl-N-{[2-fluoro-4-(3-{[(3R,6S)-1-(hydroxyacetyl)-6-methylpiperidin-3-yl]amino}-1H-pyrazolo[3,4-b]pyridin-4-yl)phenyl]methyl}-1,2,4-oxadiazole-5-carboxamide
2-[(2S,5R)-5-{[4-(4-{[(3-tert-butyl-1,2,4-oxadiazole-5-carbonyl)amino]methyl}-3-fluorophenyl)-1H-pyrazolo[3,4-b]pyridin-3-yl]amino}-2-methylpiperidin-1-yl]-2-oxoethyl acetate
N-[(4-{3-[(3R)-3-acetamidopyrrolidin-1-yl]-1H-pyrazolo[3,4-b]pyridin-4-yl}-2-fluorophenyl)methyl]-2-fluoro-4-(2-hydroxypropan-2-yl)benzamide
N-[(4-{3-[(3R)-3-acetamidopyrrolidin-1-yl]-1H-pyrazolo[3,4-b]pyridin-4-yl}-2-fluorophenyl)methyl]-5-(2-hydroxypropan-2-yl)pyridine-2-carboxamide;
or a pharmaceutically acceptable salt thereof.

96. A pharmaceutical composition comprising at least one pharmaceutically acceptable carrier and a pharmaceutically effective amount of a compound of any one of the preceding claims.

97. The pharmaceutical composition of claim 96, wherein the pharmaceutical composition is formulated for a route of administration selected from oral administration, parenteral administration, buccal administration, nasal administration, topical administration, or rectal administration.

98. The pharmaceutical composition of claim 96, wherein the carrier is a parenteral carrier.

99. The pharmaceutical composition of claim 96, wherein the carrier is an oral carrier.

100. The pharmaceutical composition of claim 96, wherein the carrier is a topical carrier.

101. A method for treating or ameliorating in a subject in need thereof a disease or condition that is associated with the activity of BTK in vivo, which comprises administering to the subject an effective amount of a compound or composition of any one of the preceding claims.

102. The method according to claim 101, wherein the disease or condition is an autoimmune disease.

103. The method of claim 102, wherein the autoimmune disease is selected from rheumatoid arthritis or lupus.

104. The method according to claim 101, wherein the disease or condition is a heteroimmune disease.

105. The method according to claim 101, wherein the disease or condition is a cancer.

106. The method of claim 105, wherein the cancer is a B-cell proliferative disorder.

107. The method of claim 106, wherein the B-cell proliferative disorder is diffuse large B cell lymphoma, follicular lymphoma or chronic lymphocytic leukemia.

108. The method according to claim 107, wherein the disease or condition is mastocytosis.

109. The method according to claim 101, wherein the disease or condition is osteoporosis or bone resorption disorder.

110. The method according to claim 101, wherein the disease or condition is an inflammatory disease.
